(19) United States
(12) Patent Application Publication
(10) Pub. No.: US 2025/0304549 A1
(43) Pub. Date: Oct. 2, 2025

(54) HYDROXYAMIDE DERIVATIVE AND USE THEREOF

(71) Applicants: SICHUAN HUIYU PHARMACEUTICAL CO., LTD., Neijiang (CN); SICHUAN HUIYU SEACROSS PHARMACEUTICAL TECHNOLOGY CO., LTD., Chengdu (CN)

(72) Inventors: Shoujun CHEN, Neijiang (CN); Jiaming YAN, Neijiang (CN); Zhao DING, Neijiang (CN); Min LI, Neijiang (CN); Haibo WANG, Neijiang (CN)

(21) Appl. No.: 18/864,895

(22) PCT Filed: May 19, 2023
(86) PCT No.: PCT/CN2023/095304

§ 371 Date: Nov. 12, 2024

(30) Foreign Application Priority Data
May 20, 2022 (CN) 202210550737.5
Nov. 3, 2022 (CN) 202211367769.8

(51) Int. Cl. C07D 401/04; A61K 31/095; A61K 31/423; A61K 31/4545; A61K 31/5377; C07D 207/04; C07D 401/14; C07D 409/12; C07D 413/14; C07D 471/10; C07D 487/04

(52) U.S. Cl. CPC C07D 401/04; A61K 31/095; A61K 31/423; A61K 31/4545; A61K 31/5377; C07D 207/04; C07D 401/14; C07D 409/12; C07D 413/14; C07D 471/10; C07D 487/04

(57) ABSTRACT

Provided in the present application is a hydroxylamide derivative represented by formula (I), and a tautomer, stereoisomer, solvate, metabolite, isotopically-labeled compound, pharmaceutically acceptable salt or co-crystal thereof. The compound provided in the present application has an inhibitory effect on both HDAC and LSD1, and can be used for treating diseases mediated by LSD1 and/or HDAC.

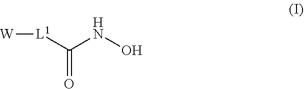

### CROSS-REFERENCE TO RELATED APPLICATIONS

The present application is a national phase filing under 35 U.S.C. § 371 of International Application No. PCT/CN2023/095304, filed on May 19, 2023, which claims the benefit of Chinese Patent Application No. 202211367769.8, filed on Nov. 3, 2022 and Chinese Patent Application No. 202210550737.5, filed on May 20, 2022, each of the applications is incorporated herein by reference in its entirety.

### FIELD OF THE INVENTION

The present disclosure belongs to the field of medicinal chemistry, and specifically discloses a hydroxylamide derivative and use thereof. This class of compounds exhibit potent inhibition effects on LSD1 and HDAC protein activities, can be used as an inhibitor for LSD1 and/or HDAC protein for treating cancer and other diseases mediated by LSD1, HDAC protein, and has a wide application potential.

### BACKGROUND

LSD1 protein (Lysine Specific Demethylase 1, Histone Demethylase, also known as KDM1A) was first discovered and reported by Shi Yang team of Harvard University in 2004 (Shi, Y., Lan, F., Matson, C., Mulligan, P., Whetstine, J. R., Cole, P. A., Casero, R. A., and Shi, Y. Histone demethylation mediated by the nuclear amine oxidase homolog LSD1. Cell 2004, 119, 941-953). As a histone demethylase involved in transcriptional regulation, LSD1 has a variety of biological functions, mainly including promoting tumor proliferation, inhibiting energy metabolism, promoting lipogenesis, inhibiting lipolysis and regulating cell differentiation, etc. Inhibition of LSD1 function can enhance the expression of endogenous retroviral elements (ERVs) and inhibit the function of RISC (RNA-induced silencing complex) complex, resulting in the overexpression of double-stranded RNA (dsRNA) and the activation of type I interferon (IFN) (Doll, S., Kriegmair, M. C., Santos, A., Wierer, M., Coscia, F., Neil, H. M., et al. Rapid proteomic analysis for solid tumors reveals LSD1 as a drug target in an end-stage cancer patient. Molecular Oncology, 2018, 12(8), 1296-1307.). At the same time, researchers found that LSD1 showed a trend of overexpression in cancers. The survival time of patients with LSD1 overexpression was significantly shortened, suggesting that LSD1 overexpression is a poor prognostic factor. In addition, LSD1 was also found to be highly expressed in various cancer tissues, and more and more reports indicated that LSD1 was involved in various tumor processes and embryonic development as an epigenetic regulator. The TCGA cancer database also showed that LSD1 expression was negatively correlated with IFN antiviral effects and CD8 T cell infiltration, which is consistent with the tests in mouse models. Therefore, inhibition of LSD1 can enhance tumor immunogenicity and promote T cell infiltration, activate anti-tumor T cell immunity, and can be used as a target for tumor therapy in combination with anti-PD-1 immunotherapy. Relevant research results also indicated that inhibition of DNA methylation alone or in combination with HDAC inhibitors can lead to the activation of tumor interferon (IFN) pathway and enhance the efficacy of tumor immunotherapy. Meanwhile, blocking DNA methylation in T cells can also enhance T cell activity and tumor suppression mediated by PD-1/PD-L1 immunotherapy (Chiappinelli, K. B., Strissel, P. L., Desrichard, A., Li, H., Henke, C., Akman, B., Hein, A., Rote, N. S., Cope, L. M., Snyder, A., et al. Inhibiting DNA methylation causes an interferon response in cancer via dsRNA including endogenous retroviruses. Cell 2015, 162, 974-986; Topper, M. J., Vaz, M., Chiappinelli, K. B., DeStefano Shields, C. E., Niknafs, N., Yen, R. C., Wenzel, A., Hicks, J., Ballew, M., Stone, M., et al. Epigenetic therapy ties MYC depletion to reversing immune evasion and treating lung cancer. Cell 2017, 171, 1284-130; Ghoneim, H. E., Fan, Y., Moustaki, A., Abdelsamed, H. A., Dash, P., Dogra, P., Carter, R., Awad, W., Neale, G., Thomas, P. G., et al. De novo epigenetic programs inhibit PD-1 blockade-mediated t cell rejuvenation. Cell 2017, 170, 142-157).

LSD1 is composed of 852 amino acids and has a molecular weight of 93 kDa. Analysis of 27 tissue samples from 95 individuals showed that LSD1 was widely expressed, with less secretion in the liver, pancreas and salivary glands, and higher expression in testicular tissues, while the expression levels in other tissues were similar. Research found that the expression level of LSD1 was significantly increased in different tumor tissues, such as neuroblastoma, breast cancer (Wang, Y.; Zhang, H.; et al, Cell 2009, 138(4), 660-72.), prostate cancer (Zhao, L.-J.; Fan, Q.-Q.; et al., Pharmacol. Res. 2020, 159, 104991), pancreatic cancer (Sehrawat, A.; Gao, L.; et al., Proc. Nat. Acad. Sci. USA 2018, 115(18), E4179-E4188.), colon cancer and glioma and blood cancer (Hatzi, K.; Geng, H.; et al., Nature Immunology 2019, 20(1), 86-96.). Moreover, high expression of LSD1 is often associated with poorer tumor prognosis and recurrence after treatment (Lynch, J.; Harris, W.; et al., Expert Opinion on Therapeutic Targets 2012, 16(12), 1239-1249.).

Researches discovered that LSD1 exerted the biological functions thereof not only by demethylating histones, but also by demethylating non-histone proteins p53 and Dnmt1. The biological roles of LSD1 are mainly manifested in the regulation of sex hormone receptor-mediated gene transcription, the regulation of tumor cell proliferation, apoptosis and metastasis, as well as the regulation of embryonic development (Ancelin, K.; Syx, L.; et al., eLife 2016, 5, e08851/1-e08851/24.), mitosis, etc. Additionally, LSD1 was reported to be associated with osteoporosis (Sun, J.; Ermann, J.; et al., Bone Res. 2018, 6(1), 1-12); in addition, LSD1 inhibition was found to be associated with macrophage phenotypic polarization (Tan, A. H. Y.; Tu, W. J.; et al., Front. Immunol. 2019, 10, 1351.) and CD8+ T-cell infiltration in the tumor microenvironment (Hatzi, K.; Geng, H.; et al., Nature Immunology 2019, 20(1), 86-96). Therefore, the development of new LSD1 inhibitors has drawn great attention in cancer research.

Histone deacetylase (HDAC) is involved in histone acetylation, binding to deacetylated proteins and interacting with nonhistone proteins. HDAC has a wide range of biological functions, including neurodegeneration, inflammation, metabolic disorders, tumorigenesis, etc. HDAC1 is a possible prognostic marker for lung cancer and breast cancer and is overexpressed in prostate cancer, gastric cancer and colon cancer; HDAC2 is commonly overexpressed in colorectal cancer and gastric cancer; HDAC3 expression is elevated in lung cancer and most solid tumors; HDAC6 is mainly overexpressed in breast cancer; knockdown of HDAC8 can inhibit tumor cell growth and proliferation in various human tumor cells. In cancer cells, overexpression of HDACs leads to enhanced deacetylation and unfavorable expression of specific genes, including some tumor suppressor genes. So far, five HDAC inhibitors (HDACis) have been approved for marketing, namely Vorinostat (SAHA), Romidepsin (FK228), Belinostat (PXD-101), Panobinostat (LBH-589) and Chidamide, for treating various tumors such as malignant lymphoma, myeloma, hematologic cancers, and pancreatic cancer. Additionally, there are also several HDACs inhibitor candidates in clinical trials. The LSD1 and HDACs both play important roles in the occurrence and development of certain tumors, and in various cancers such as bladder, breast and lung cancers, reducing LSD1 expression or inhibiting LSD1 activity can significantly enhance the sensitivity of cancer cells to HDACs inhibitors. Duan, et al., reported that simultaneous inhibition of the activities of LSD1 and HDACs with small molecule inhibitors had synergistic antitumor effects (Duan, Y. C.; et al, Eur J Med Chem. 2021, 220, 113453. doi: 10.1016/j.ejmech.2021.113453.). Meanwhile, studies showed that dual-targeted drugs had more predictable complex metabolic pathways, better PK/PD properties, and better bioavailability than multidrug combinations (Giulia S.; et al, Current Opinion in Chemical Biology 2019, 50, 89-100). In addition, dual-targeted drugs can ensure that the dual pharmacodynamic moieties can synchronize their effects at the same time in the same cell, as opposed to a combination of drugs. (de Lera, A. R.; Ganesan, A., Clin Epigenetics 2016, 8:105.) Additional advantages of dual-targeted monotherapy include improved patient compliance and lower medication costs. (Fu, R. G., Sun, Y., Sheng, W. B., Liao, D. F., Eur. J. Med. Chem. 2017, 136, 195-211) Cole and colleagues recently reported a class of LSD1/HDAC1 dual-targeted inhibitors that showed good in vivo activity in a mouse model of melanoma (Kalin, J. H.; et al; Nat. Commun., 2018, 9, 53). Other academic institutions such as Xinxiang Medical College have recently disclosed several dual LSD1/HDAC inhibitor patents (CN111592487; CN113444038; CN113527195). There is still unmet medical needs for the development of novel dual inhibitors that inhibit both HDAC and LSD1.

### SUMMARY OF THE INVENTION

The present disclosure provides a compound represented by formula (I) or a tautomer, stereoisomer, solvate, metabolite, isotopically-labeled compound, pharmaceutically acceptable salt or co-crystal thereof,

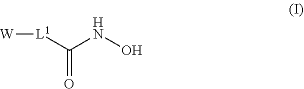

wherein,
L¹ is selected from a bond, —C₁₋₁₀ alkyl-, —C₂₋₆ alkenyl-, —C₁₋₁₀ alkyl-C₂₋₆ alkenyl-, —C₂₋₆ alkynyl-, —C₆₋₁₀ heteroaryl-, —C₁₋₁₀ alkyl-(C₆₋₁₀ aryl or heteroaryl or heterocycloalkyl or heterocycloalkenyl)-C₂₋₆ alkenyl-, —(C₆₋₁₀ aryl or heteroaryl)-C₂₋₆ alkenyl-, —C₁₋₁₀ alkyl-(C₆₋₁₀ aryl)-, —C₁₋₁₀ alkyl-(C₆₋₁₀ aryl)-C₁₋₁₀ alkyl-, —NR^{a}—, —C₁₋₁₀ alkyl-(C₆₋₁₀ aryl)-C₂₋₆ alkynyl-, —C₁₋₁₀ alkyl-(C₆₋₁₀ heterocycloalkyl)-(C₆₋₁₀ aryl)-, —C₁₋₁₀ alkyl-NH-6-10-membered heteroaryl-, —C₁₋₁₀ alkyl-6-10-membered heteroaryl-, —C₁₋₁₀ alkyl-C₆₋₁₀ cycloalkenyl-C₂₋₆ alkenyl-, —C₁₋₁₀ alkyl-C₆₋₁₀ aryl-C₃₋₆ cycloalkenyl-, —C₁₋₁₀ alkyl-C₆₋₁₀ aryl-C₃₋₆ cycloalkyl-, —C₁₋₁₀ alkyl-O—C₆₋₁₀ aryl-, —C₁₋₁₀ alkyl-6-10-membered heteroaryl-C₁₋₁₀ alkyl-, —C₁₋₁₀ alkyl-6-10-membered aryl-O—C₁₋₁₀ alkyl-, —C₁₋₁₀ alkyl-6-10-membered heteroaryl-O—C₁₋₁₀ alkyl-, —C₁₋₁₀ alkyl-6-10-membered aryl-S—C₁₋₁₀ alkyl-, the alkyl, alkenyl, cycloalkyl, heterocycloalkyl, heterocycloalkenyl, aryl or heteroaryl is optionally substituted with one or more substituents selected from hydrogen, halogen, CN, CF₃, hydroxyl, C₁₋₆ alkyl, C₁₋₆ alkoxy, C₂₋₆ alkenyl, C₂₋₆ alkynyl, —NR^{a}R^{b}, COOH, —C(═O)NR^{a}R^{b}, the heteroaryl, heterocycloalkyl contain 1 to 4 heteroatoms optionally selected from N, O, or S; alternatively, one or more alkyl groups of the alkyl may optionally be replaced by one or more groups selected from —C(═O)—, —S(═O)₂— or —NR^{a}-;
preferably, L¹ is selected from a bond, —C₁₋₁₀ alkyl-, —C₂₋₆ alkenyl-, —C₁₋₁₀ alkyl-C₂₋₆ alkenyl-, —C₂₋₆ alkynyl-, —C₆₋₁₀ heteroaryl-, —C₁₋₁₀ alkyl-(C₆₋₁₀ aryl or heteroaryl or heterocycloalkyl or heterocycloalkenyl)-C₂₋₆ alkenyl-, —(C₆₋₁₀ aryl or heteroaryl)-C₂₋₆ alkenyl-, —C₁₋₁₀ alkyl-(C₆₋₁₀ aryl)-, —C₁₋₁₀ alkyl-(C₆₋₁₀ aryl)-C₁₋₁₀ alkyl-, —NR^{a}—, —C₁₋₁₀ alkyl-(C₆₋₁₀ aryl)-C₂₋₆ alkynyl-, —C₁₋₁₀ alkyl-(C₆₋₁₀ heterocycloalkyl)-(C₆₋₁₀ aryl)-, —C₁₋₁₀ alkyl-NH-6-10-membered heteroaryl-, —C₁₋₁₀ alkyl-6-10-membered heteroaryl-, —C₁₋₁₀ alkyl-C₆₋₁₀ cycloalkenyl-C₂₋₆ alkenyl-, —C₁₋₁₀ alkyl-C₆₋₁₀ aryl-C₃₋₆ cycloalkenyl-, —C₁₋₁₀ alkyl-C₆₋₁₀ aryl-C₃₋₆ cycloalkyl-, —C₁₋₁₀ alkyl-O—C₆₋₁₀ aryl-, —C₁₋₁₀ alkyl-6-10-membered heteroaryl-O—C₁₋₁₀ alkyl-, the alkyl, alkenyl, cycloalkyl, heterocycloalkyl, heterocycloalkenyl, aryl or heteroaryl is optionally substituted with one or more substituents selected from hydrogen, halogen, CN, CF₃, hydroxyl, C₁₋₆ alkyl, C₁₋₆ alkoxy, C₂₋₆ alkenyl, C₂₋₆ alkynyl, —NR^{a}R^{b}, COOH, —C(═O)NR^{a}R^{b}, the heteroaryl, heterocycloalkyl contain 1 to 4 heteroatoms optionally selected from N, O, or S; alternatively one or more alkyl groups of the alkyl may optionally be replaced by one or more groups selected from —C(═O)—, —S(═O)₂— or —NR^{a}—;
   preferably, the left end group of L¹ is connected to W, the right end group of L¹ is connected to

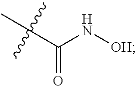 [SMILES: *C(=O)NO]

W is selected from:

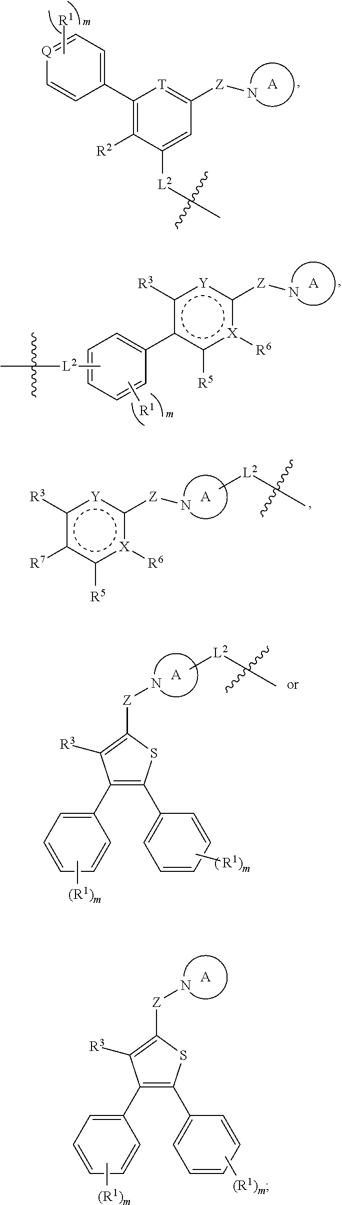

L² is selected from a bond, —O—, —C(═O)—, —NR^{a}—, —CH₂—NR^{a}—, —NR^{a}—C(O)—, —NR^{a}—S(═O)₂—, —S— or —S(═O)₂—;
preferably, L² is selected from a bond, —O—, —C(═O)—, —NR^{a}—, —NR^{a}—C(O)—, —NR^{a}—S(═O)₂—, —S— or —S(═O)₂—;
ring A is selected from nitrogen-containing C₃₋₁₀ heteroaryl, C₃₋₁₀ heterocycloalkyl or C₃₋₁₀ heterocycloalkenyl, wherein, the heteroaryl, heterocycloalkyl, heterocycloalkenyl are optionally substituted with one or more R⁴, the heteroaryl, heterocycloalkyl contain 1 to 4 heteroatoms optionally selected from N, O, or S;
R⁴ is each independently selected at each occurrence from hydrogen, halogen, CN, hydroxyl, C₁₋₆ alkyl, C₁₋₆ alkoxy, C₂₋₆ alkenyl, C₂₋₆ alkynyl, COOH, —NR^{a}R^{b}, —C(═O)NR^{a}R^{b}; alternatively, when R⁴ is selected from C₁₋₆ alkyl, any two R⁴ and the atom to which they connect can collectively form a 5 to 10-membered heteroalicyclic;
R¹, R⁶ are each independently selected at each occurrence from hydrogen, halogen, CN, hydroxyl, C₁₋₆ alkyl, C₁₋₆ alkyl-CN, C₁₋₆ alkoxy, hydroxyl-substituted C₁₋₆ alkyl, halogen-substituted C₁₋₆ alkyl, halogen-substituted C₁₋₆ alkoxy, C₃₋₆ cycloalkyl, C₂₋₆ alkenyl, C₂₋₆ alkynyl, COOH, —NR^{a}R^{b}, —C(═O)NR^{a}R^{b}, —S(═O)₂R^{a}, —C₂₋₆ alkenyl-C(═O)NR^{a}R^{b};
preferably, R¹, R⁶ are each independently selected at each occurrence from hydrogen, halogen, CN, hydroxyl, C₁₋₆ alkyl, C₁₋₆ alkoxy, hydroxyl-substituted C₁₋₆ alkyl, halogen-substituted C₁₋₆ alkyl, halogen-substituted C₁₋₆ alkoxy, C₃₋₆ cycloalkyl, C₂₋₆ alkenyl, C₂₋₆ alkynyl, COOH, —NR^{a}R^{b}, —C(═O)NR^{a}R^{b}, —S(═O)₂R^{a}, —C₂₋₆ alkenyl-C(═O)NR^{a}R^{b};
R², R³, R⁷ are each independently selected from hydrogen, C₁₋₆ alkyl, C₁₋₆ alkoxy, C₆₋₁₀ aryl or C₆₋₁₀ heteroaryl, and R³ and R⁷ are not both hydrogen, wherein, the aryl or heteroaryl is optionally substituted with one or more substituents selected from hydrogen, halogen, CN, CF₃, hydroxyl, C₁₋₆ alkyl, C₁₋₆ alkoxy, C₂₋₆ alkenyl, C₂₋₆ alkynyl, C₃₋₆ cycloalkyl, halogen-substituted C₁₋₆ alkoxy, halogen-substituted C₁₋₆ alkyl, hydroxyl-substituted C₁₋₆ alkyl, hydroxyl-substituted C₁₋₆ alkoxy, hydroxyl-substituted C₁₋₆ alkoxy-C₃₋₆ cycloalkyl, COOH, —NR^{a}R^{b}, —S(═O)₂R^{a}, —C(═O)NR^{a}R^{b}, —C₂₋₆ alkenyl-C(═O)NR^{a}R^{b}, 3 to 6-membered heterocycloalkyl or heterocycloalkenyl, wherein the heteroaryl, heterocycloalkyl contains 1 to 4 heteroatoms optionally selected from N, O, or S;
preferably, R², R³, R⁷ are each independently selected from hydrogen, C₁₋₆ alkyl, C₁₋₆ alkoxy, C₆₋₁₀ aryl or C₆₋₁₀ heteroaryl, and R³ and R⁷ are not both hydrogen, wherein, the aryl or heteroaryl is optionally substituted with one or more substituents selected from hydrogen, halogen, CN, CF₃, hydroxyl, C₁₋₆ alkyl, C₁₋₆ alkoxy, C₂₋₆ alkenyl, C₂₋₆ alkynyl, C₃₋₆ cycloalkyl, halogen-substituted C₁₋₆ alkoxy, hydroxyl-substituted C₁₋₆ alkyl, hydroxyl-substituted C₁₋₆ alkoxy, COOH, —NR^{a}R^{b}, —S(═O)₂R^{a}, —C(═O)NR^{a}R^{b}, —C₂₋₆ alkenyl-C(═O)NR^{a}R^{b}, 3 to 6-membered heterocycloalkyl, heterocycloalkenyl, the heteroaryl, heterocycloalkyl contain 1 to 4 heteroatoms optionally selected from N, O, or S;
m is selected from 0, 1, 2, 3, 4 or 5;
Q, T are each independently selected from N or C;
X, Y are each independently selected from C and N;
Z is selected from a bond, —CH₂—, —C(═O) or —S(═O)₂—;
preferably, R⁵ is each independently selected at each occurrence from hydrogen, halogen, CN, hydroxyl, C₁₋₆ alkyl, C₁₋₆ alkoxy, C₂₋₆ alkenyl, C₂₋₆ alkynyl, ═O, COOH, —NR^{a}R^{b}, —C(═O)NR^{a}R^{b}, C₃₋₆ cycloalkyl, 3 to 6-membered heterocycloalkyl, C₆₋₁₀ aryl or C₆₋₁₀ heteroaryl, wherein, the alkyl, alkoxy, alkenyl, alkynyl, cycloalkyl, heterocycloalkyl, aryl or heteroaryl is optionally substituted with one or more substituents selected from hydrogen, halogen, CN, NO₂, CF₃, CHF₂, hydroxyl, C₁₋₆ alkyl, C₁₋₆ alkoxy, —C(═O)—C₁₋₆ alkoxy, C₂₋₆ alkenyl, C₂₋₆ alkynyl, COOH, —NR^{a}R^{b}, —C(═O)NR^{a}R^{b}, the heteroaryl, heterocycloalkyl contain 1 to 4 heteroatoms optionally selected from N, O, or S;
R⁵ is each independently selected at each occurrence from hydrogen, halogen, CN, hydroxyl, C₁₋₆ alkyl, C₁₋₆ alkoxy, C₂₋₆ alkenyl, C₂₋₆ alkynyl, ═O, COOH, —NR^{a}R^{b}, —C(═O)NR^{a}R^{b}, C₃₋₆ cycloalkyl, 3 to 6-membered heterocycloalkyl, C₆₋₁₀ aryl or C₆₋₁₀ heteroaryl, wherein, the alkyl, alkoxy, alkenyl, alkynyl, cycloalkyl, heterocycloalkyl, aryl or heteroaryl is optionally substituted with one or more substituents selected from hydrogen, halogen, CN, NO₂, CF₃, hydroxyl, C₁₋₆ alkyl, C₁₋₆ alkoxy, C₂₋₆ alkenyl, C₂₋₆ alkynyl, COOH, —NR^{a}R^{b} or —C(═O)NR^{a}R^{b}, the heteroaryl, heterocycloalkyl contain 1 to 4 heteroatoms optionally selected from N, O, or S;
alternatively, R⁵, R⁶ and the atom to which both of them directly connect collectively form cycloalkyl, heteroalicyclic, aryl or heteroaryl, wherein, the cycloalkyl, heteroalicyclic, aryl, heteroaryl are optionally substituted with one or more substituents selected from hydrogen, halogen, CN, hydroxyl, C₁₋₆ alkyl, C₁₋₆ alkoxy, C₂₋₆ alkenyl, C₂₋₆ alkynyl, —NR^{a}R^{b}, —C(═O)NR^{a}R^{b};
R^{a}, R^{b} are each independently selected at each occurrence from hydrogen, hydroxyl, C₁₋₆ alkyl, C₁₋₆ alkoxy, C₂₋₆ alkenyl, C₂₋₆ alkynyl, C₃₋₆ cycloalkyl, halogen-substituted C₁₋₆ alkyl, 3 to 6-membered heterocycloalkyl, C₆₋₁₀ aryl or C₆₋₁₀ heteroaryl, the heteroaryl, heterocycloalkyl contain 1 to 4 heteroatoms optionally selected from N, O, or S;
[symbol] indicates a double bond may be present or not present at any position within the ring.

The present disclosure provides a compound represented by formula (I) or a tautomer, stereoisomer, solvate, metabolite, isotopically-labeled compound, pharmaceutically acceptable salt or co-crystal thereof,
wherein,
L¹ is selected from a bond, —C₁₋₁₀ alkyl-, —C₂₋₆ alkenyl-, —C₁₋₁₀ alkyl-C₂₋₆ alkenyl-, —C₂₋₆ alkynyl-, —C₆₋₁₀ heteroaryl-, —C₁₋₁₀ alkyl-(C₆₋₁₀ aryl or heteroaryl or heterocycloalkyl or heterocycloalkenyl)-C₂₋₆ alkenyl-, —(C₆₋₁₀ aryl or heteroaryl)-C₂₋₆ alkenyl-, —C₁₋₁₀ alkyl-(C₆₋₁₀ aryl)-, —C₁₋₁₀ alkyl-(C₆₋₁₀ aryl)-C₁₋₁₀ alkyl-, —NR^{a}—, —C₁₋₁₀ alkyl-(C₆₋₁₀ aryl)-C₂₋₆ alkynyl-, —C₁₋₁₀ alkyl-(C₆₋₁₀ heterocycloalkyl)-(C₆₋₁₀ aryl)-, the alkyl, alkenyl, cycloalkyl, heterocycloalkyl, heterocycloalkenyl, aryl or heteroaryl is optionally substituted with one or more substituents selected from hydrogen, halogen, CN, CF₃, hydroxyl, C₁₋₆ alkyl, C₁₋₆ alkoxy, C₂₋₆ alkenyl, C₂₋₆ alkynyl, —NR^{a}R^{b}, COOH, —C(═O)NR^{a}R^{b}, the heteroaryl, heterocycloalkyl contain 1 to 4 heteroatoms optionally selected from N, O, or S; alternatively, one or more alkyl groups of the alkyl may optionally be replaced by one or more groups selected from —C(═O)—, —S(═O)₂— or —NR^{a}-;
W is selected from:

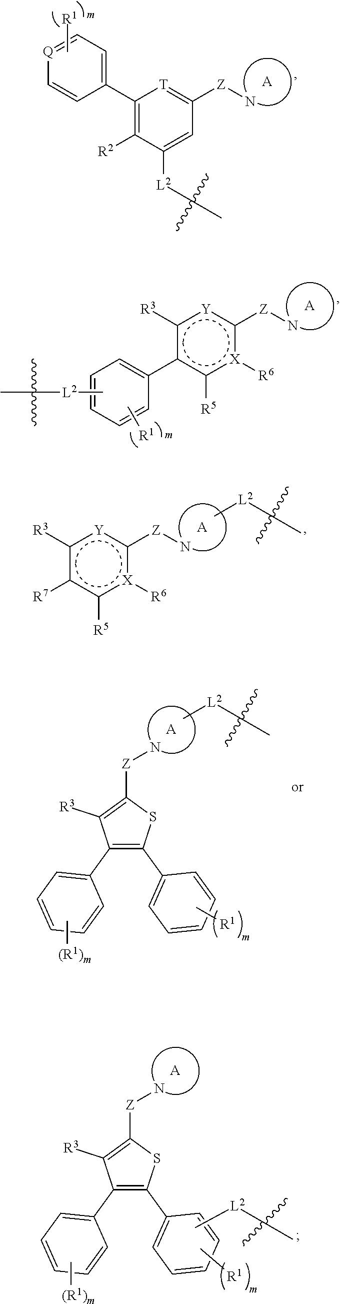

L² is selected from a bond, —O—, —C(═O)—, —NR^{a}—, —S— or —S(═O)₂—;
ring A is selected from nitrogen-containing C₃₋₁₀ heteroaryl, C₃₋₁₀ heterocycloalkyl or C₃₋₁₀ heterocycloalkenyl, wherein, the heteroaryl, heterocycloalkyl, heterocycloalkenyl are optionally substituted with one or more R⁴, the heteroaryl, heterocycloalkyl contain 1 to 4 heteroatoms optionally selected from N, O, or S;
R⁴ is each independently selected at each occurrence from hydrogen, halogen, CN, hydroxyl, C₁₋₆ alkyl, C₁₋₆ alkoxy, C₂₋₆ alkenyl, C₂₋₆ alkynyl, COOH, —NR^{a}R^{b}, —C(═O)NR^{a}R^{b}; alternatively, when R⁴ is selected from C₁₋₆ alkyl, any two R⁴ and the atom to which they connect can collectively form a 5 to 10-membered heteroalicyclic;
R¹, R⁶ are each independently selected at each occurrence from hydrogen, halogen, CN, hydroxyl, C₁₋₆ alkyl, C₁₋₆ alkoxy, hydroxyl-substituted C₁₋₆ alkyl, halogen-substituted C₁₋₆ alkyl, halogen-substituted C₁₋₆ alkoxy, C₃₋₆ cycloalkyl, C₂₋₆ alkenyl, C₂₋₆ alkynyl, COOH, —NR^{a}R^{b}, —C(═O)NR^{a}R^{b}, —S(═O)₂R^{a}, —C₂₋₆ alkenyl-C(═O)NR^{a}R^{b};
R², R³, R⁷ are each independently selected from hydrogen, C₁₋₆ alkyl, C₁₋₆ alkoxy, C₆₋₁₀ aryl or C₆₋₁₀ heteroaryl, R³ and R⁷ are not both hydrogen, wherein, the aryl or heteroaryl is optionally substituted with one or more substituents selected from hydrogen, halogen, CN, CF₃, hydroxyl, C₁₋₆ alkyl, C₁₋₆ alkoxy, C₂₋₆ alkenyl, C₂₋₆ alkynyl, COOH, —NR^{a}R^{b}, —S(═O)₂R^{a}, —C(═O)NR^{a}R^{b}, —C₂₋₆ alkenyl-C(═O)NR^{a}R^{b}, C₃₋₆ cycloalkyl, 3 to 6-membered heterocycloalkyl, heterocycloalkenyl, the heteroaryl, heterocycloalkyl contain 1 to 4 heteroatoms optionally selected from N, O, or S;
m is selected from 0, 1, 2, 3, 4 or 5;
Q, T are each independently selected from N or C;
X, Y are each independently selected from C, N, —NR^{c}— or —CR^{d}—;
Z is selected from a bond, —CH₂—, —C(═O) or —S(═O)₂—;
R⁵ is each independently selected at each occurrence from hydrogen, halogen, CN, hydroxyl, C₁₋₆ alkyl, C₁₋₆ alkoxy, C₂₋₆ alkenyl, C₂₋₆ alkynyl, ═O, COOH, —NR^{a}R^{b}, —C(═O)NR^{a}R^{b}, C₃₋₆ cycloalkyl, 3 to 6-membered heterocycloalkyl, C₆₋₁₀ aryl or C₆₋₁₀ heteroaryl, wherein, the alkyl, alkoxy, alkenyl, alkynyl, cycloalkyl, heterocycloalkyl, aryl or heteroaryl is optionally substituted with one or more substituents selected from hydrogen, halogen, CN, CF₃, hydroxyl, C₁₋₆ alkyl, C₁₋₆ alkoxy, C₂₋₆ alkenyl, C₂₋₆ alkynyl, COOH, —NR^{a}R^{b}, —C(═O)NR^{a}R^{b}, the heteroaryl, heterocycloalkyl contain 1 to 4 heteroatoms optionally selected from N, O, or S;
alternatively, R⁵, R⁶ and the atom to which both of them directly connect collectively form cycloalkyl, heteroalicyclic, aryl or heteroaryl, wherein, the cycloalkyl, heteroalicyclic, aryl, heteroaryl are optionally substituted with one or more substituents selected from hydrogen, halogen, CN, hydroxyl, C₁₋₆ alkyl, C₁₋₆ alkoxy, C₂₋₆ alkenyl, C₂₋₆ alkynyl, —NR^{a}R^{b}, —C(═O)NR^{a}R^{b};
R^{a}, R^{b} are each independently selected at each occurrence from hydrogen, hydroxyl, C₁₋₆ alkyl, C₁₋₆ alkoxy, C₂₋₆ alkenyl, C₂₋₆ alkynyl, C₃₋₆ cycloalkyl, halogen-substituted C₁₋₆ alkyl, 3 to 6-membered heterocycloalkyl, C₆₋₁₀ aryl or C₆₋₁₀ heteroaryl, the heteroaryl, heterocycloalkyl contain 1 to 4 heteroatoms optionally selected from N, O, or S;
R^{c}, R^{d} are each independently selected at each occurrence from a bond, H, C₁₋₆ alkyl, C₁₋₆ alkoxy, C₂₋₆ alkenyl, C₂₋₆ alkynyl or C₃₋₆ cycloalkyl;
[symbol] indicates a double bond may be present or not present at any position within the ring.

The present disclosure discloses the compound represented by formula (I) or automer, stereoisomer, solvate, metabolite, isotopically-labeled compound, pharmaceutically acceptable salt or co-crystal thereof, wherein, the compound is represented by formula (II), (III), (IV), (V) or (VI):

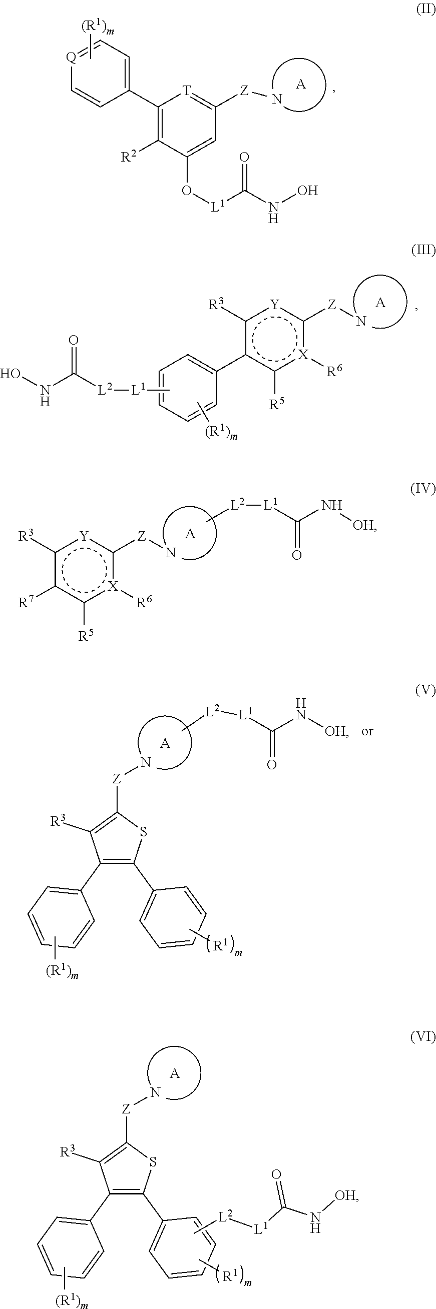

wherein the definition of each substituent in formula (II), (III), (IV), (V), or (VI) is consistent with the definition above.

The present disclosure provides the compound represented by formula (II-1):

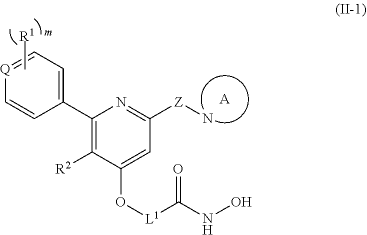

wherein,
L¹ is selected from —C₁₋₁₀ alkyl-, —C₂₋₆ alkenyl-, —C₁₋₁₀ alkyl-C₂₋₆ alkenyl-, —C₂₋₆ alkynyl-, —C₁₋₁₀ alkyl-(C₆₋₁₀ aryl)-C₂₋₆ alkenyl-, the alkyl, alkenyl are optionally substituted with one or more substituents selected from hydrogen, halogen, CN, CF₃, hydroxyl, C₁₋₆ alkyl, C₁₋₆ alkoxy.

Preferably, L¹ is selected from —C₁₋₁₀ alkyl-, —C₁₋₁₀ alkyl-C₂₋₆ alkenyl-, —C₁₋₁₀ alkyl-(C₆₋₁₀ aryl)-C₂₋₆ alkenyl-.

Preferably, L¹ is selected from —C₁₋₁₀ alkyl-, —C₁₋₁₀ alkyl-(C₆₋₁₀ aryl)-C₂₋₆ alkenyl-.

In some embodiments, L¹ is selected from —C₁₋₆ alkyl-, —C₁₋₆ alkyl-phenylene-C₂₋₆ alkenyl-.

In one aspect of the present disclosure, wherein,
ring A is selected from nitrogen-containing C₃₋₁₀ heteroaryl or C₃₋₁₀ heterocycloalkyl, wherein, the heteroaryl, heterocycloalkyl are optionally substituted with one or more R⁴.

Preferably, ring A is selected from nitrogen-containing C₃₋₁₀ heterocycloalkyl, wherein, the heterocycloalkyl is optionally substituted with one or more R⁴.

Preferably, ring A is selected from:

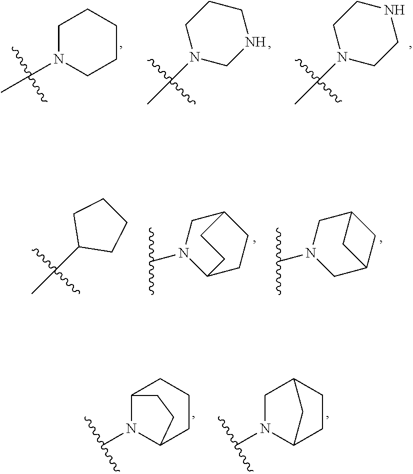

wherein the

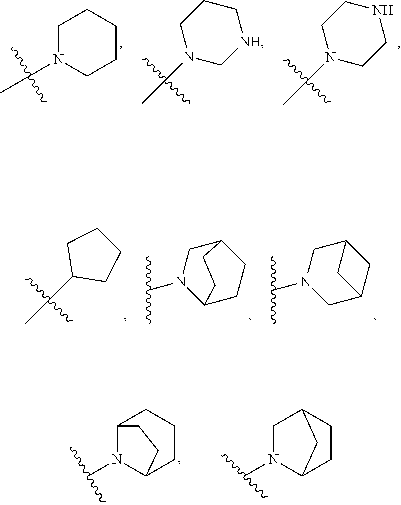

are optionally substituted with R⁴;
preferably, ring A is selected from:

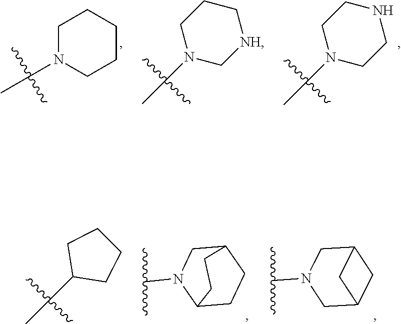

wherein the

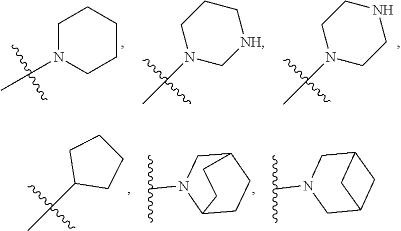

are optionally substituted with R⁴.

Preferably, ring A is selected from:

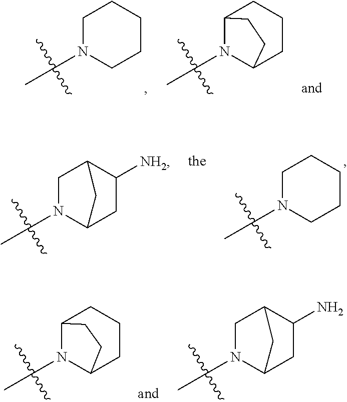 [SMILES: *N1CCCCC1 ; *N1C2CCCC1CC2 ; *N1CC2CC1CC2N ; *N1CCCCC1 ; *N1C2CCCC1CC2 ; *N1CC2CC1CC2N]

are optionally substituted with R⁴.

Preferably, ring A is selected from:

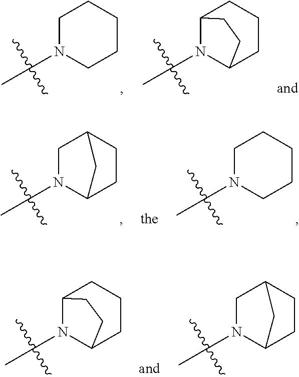 [SMILES: *N1CCCCC1 ; *N1C2CCCC1CC2 ; *N1CC2CCC1C2 ; *N1CCCCC1 ; *N1C2CCCC1CC2 ; *N1CC2CCC1C2]

are optionally substituted with R⁴.

Preferably, ring A is selected from

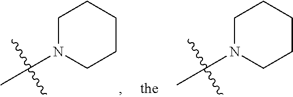 [SMILES: *N1CCCCC1 ; *N1CCCCC1]

is optionally substituted with R⁴.

In one aspect of the present disclosure, wherein,
R⁴ is each independently selected at each occurrence from hydrogen, halogen, CN, hydroxyl, C₁₋₆ alkyl, C₁₋₆ alkoxy, C₂₋₆ alkenyl, C₂₋₆ alkynyl, COOH, —NR^{a}R^{b}, —C(═O)NR^{a}R^{b}.

Preferably, R⁴ is each independently selected at each occurrence from hydrogen, halogen, CN, hydroxyl, C₁₋₆ alkyl, C₁₋₆ alkoxy, —NR^{a}R^{b}.

Preferably, R⁴ is selected from hydrogen, —NR^{a}R^{b}, C₁₋₆ alkyl.

Preferably, R⁴ is selected from hydrogen, —NR^{a}R^{b}.

In one aspect of the present disclosure, wherein,
R¹ is selected from hydrogen, halogen, CN, hydroxyl, C₁₋₆ alkyl, C₁₋₆ alkoxy, hydroxyl-substituted C₁₋₆ alkyl, halogen-substituted C₁₋₆ alkyl, halogen-substituted C₁₋₆ alkoxy, C₃₋₆ cycloalkyl, C₂₋₆ alkenyl, C₂₋₆ alkynyl.

Preferably, R¹ is selected from hydrogen, halogen, CN, C₁₋₆ alkyl.

Preferably, R¹ is selected from hydrogen, halogen, CN.

R² is selected from hydrogen, halogen, CN, hydroxyl, C₁₋₆ alkyl, C₁₋₆ alkoxy, C₂₋₆ alkenyl, C₂₋₆ alkynyl, COOH, —NR^{a}R^{b}, C₆₋₁₀ aryl or C₆₋₁₀ heteroaryl, wherein, the alkyl, alkoxy, alkenyl, aryl or heteroaryl is optionally substituted with one or more substituents selected from hydrogen, halogen, CN, hydroxyl, C₁₋₆ alkyl, C₁₋₆ alkoxy, C₂₋₆ alkenyl, C₂₋₆ alkynyl, COOH, —NR^{a}R^{b}, —S(═O)₂R^{a}, —O—C₁₋₆ alkyl-OH, the heteroaryl, heterocycloalkyl contain 1 to 4 heteroatoms optionally selected from N, O, or S;
R² is selected from hydrogen, halogen, CN, hydroxyl, C₁₋₆ alkyl, C₁₋₆ alkoxy, C₂₋₆ alkenyl, C₂₋₆ alkynyl, COOH, —NR^{a}R^{b}, C₆₋₁₀ aryl or C₆₋₁₀ heteroaryl, wherein, the alkyl, alkoxy, alkenyl, aryl or heteroaryl is optionally substituted with one or more substituents selected from hydrogen, halogen, CN, hydroxyl, C₁₋₆ alkyl, C₁₋₆ alkoxy, C₂₋₆ alkenyl, C₂₋₆ alkynyl, COOH, —NR^{a}R^{b}, —S(═O)₂R^{a}, the heteroaryl, heterocycloalkyl contain 1 to 4 heteroatoms optionally selected from N, O, or S.

In some embodiments, R² is selected from hydrogen, C₆₋₁₀ aryl or C₆₋₁₀ heteroaryl, wherein, the aryl or heteroaryl is optionally substituted with one or more substituents selected from hydrogen, halogen, hydroxyl, C₁₋₆ alkyl, C₁₋₆ alkoxy, —NR^{a}R^{b}, —S(═O)₂R^{a}, —O—C₁₋₆ alkyl-OH, the heteroaryl, heterocycloalkyl contain 1 to 4 heteroatoms optionally selected from N, O, or S;
preferably, R² is selected from hydrogen, C₆₋₁₀ aryl or C₆₋₁₀ heteroaryl, wherein, the aryl or heteroaryl is optionally substituted with one or more substituents selected from hydrogen, halogen, hydroxyl, C₁₋₆ alkyl, C₁₋₆ alkoxy, —NR^{a}R^{b}, —S(═O)₂R^{a}, the heteroaryl, heterocycloalkyl contain 1 to 4 heteroatoms optionally selected from N, O, or S.

In some embodiments, R² is selected from hydrogen,

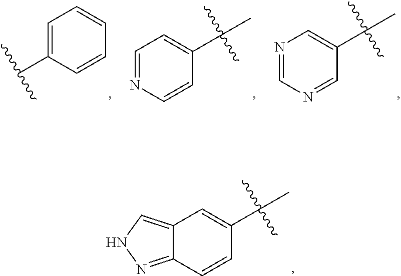

wherein, the

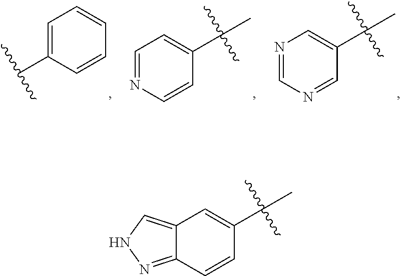

are optionally substituted with one or more substituents selected from hydrogen, halogen, hydroxyl, C₁₋₆ alkyl, C₁₋₆ alkoxy, —NR^{a}R^{b}, —S(═O)₂—R^{a}, —O—C₁₋₆ alkyl-OH;
preferably, R² is selected from hydrogen,

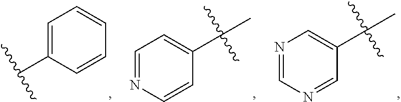

wherein, the

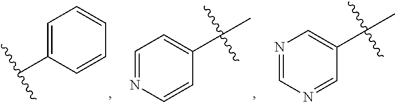

are optionally substituted with one or more substituents selected from hydrogen, halogen, hydroxyl, C₁₋₆ alkyl, C₁₋₆ alkoxy, —NR^{a}R^{b}, —S(═O)₂—R^{a}.

Preferably, R² is selected from

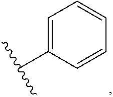

wherein, the

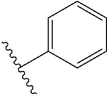 [SMILES: *c1ccccc1]

is optionally substituted with one or more substituents selected from hydrogen, hydroxyl, C₁₋₆ alkoxy.

In one aspect of the present disclosure, wherein,
m is selected from 0, 1, 2 or 3; preferably, m is selected from 1 or 2; more preferably, m is 2.
Q is each independently selected from N or C; preferably, Q is selected from C.

In one aspect of the present disclosure, wherein,
R^{a}, R^{b} are each independently selected at each occurrence from hydrogen, hydroxyl, C₁₋₆ alkyl, C₁₋₆ alkoxy, C₂₋₆ alkenyl, C₂₋₆ alkynyl, C₃₋₆ cycloalkyl, halogen-substituted C₁₋₆ alkyl.

Preferably, R^{a}, R^{b} are each independently selected at each occurrence from hydrogen, hydroxyl, C₁₋₆ alkyl, C₁₋₆ alkoxy, C₂₋₆ alkenyl, C₂₋₆ alkynyl, C₃₋₆ cycloalkyl.

Preferably, R^{a}, R^{b} are each independently selected at each occurrence from hydrogen and methyl.

Preferably, R^{a}, R^{b} are each independently selected from hydrogen.

The present disclosure provides the compound represented by formula (III-1):

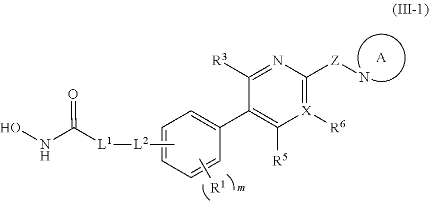

wherein,
X is selected from C and N;
L¹ is selected from a bond, —C₁₋₁₀ alkyl-, —C₂₋₆ alkenyl-, —C₁₋₁₀ alkyl-C₂₋₆ alkenyl-, —C₂₋₆ alkynyl-, —C₁₋₁₀ alkyl-(C₆₋₁₀ aryl or heteroaryl)-C₂₋₆ alkenyl-, —C₁₋₁₀ alkyl-(C₆₋₁₀ aryl)-, —NR^{a}—, the alkyl, alkoxy, alkenyl, aryl or heteroaryl is optionally substituted with one or more substituents selected from hydrogen, halogen, CN, CF₃, hydroxyl, C₁₋₆ alkyl, C₁₋₆ alkoxy, C₂₋₆ alkenyl, C₂₋₆ alkynyl, —NR^{a}R^{b}, COOH;
alternatively, one or more carbon atoms in the alkyl can optionally be replaced by one or more groups selected from —NH—.

Preferably, L¹ is selected from a bond, —C₁₋₁₀ alkyl-, —C₂₋₆ alkenyl-, —C₁₋₁₀ alkyl-C₂₋₆ alkenyl-, —C₁₋₁₀ alkyl-(C₆₋₁₀ aryl or heteroaryl)-C₂₋₆ alkenyl-, —C₁₋₁₀ alkyl-(C₆₋₁₀ aryl)-, the alkyl, alkoxy, alkenyl, aryl or heteroaryl is optionally substituted with one or more substituents selected from hydrogen, halogen, CN, CF₃, hydroxyl, C₁₋₆ alkoxy, C₂₋₆ alkenyl, —NR^{a}R^{b};

alternatively, one or more carbon atoms in the alkyl can optionally be replaced by one or more groups selected from —NH—.

Preferably, L¹ is selected from —C₁₋₁₀ alkyl-, —C₁₋₁₀ alkyl-(C₆₋₁₀ aryl or heteroaryl)-C₂₋₆ alkenyl-, —C₁₋₁₀ alkyl-(C₆₋₁₀ aryl)-, the alkyl, aryl or heteroaryl is optionally substituted with one or more substituents selected from hydrogen, halogen, CN, CF₃, hydroxyl, C₁₋₆ alkyl, C₁₋₆ alkoxy;

preferably, L¹ is-C₁₋₆ alkyl-.

In one aspect of the present disclosure, wherein, L² is selected from a bond, —O—, —C(═O)—, —NR^{a}—, —NR^{a}—C(O)— or —S(═O)₂—;
in some embodiments, L² is selected from a bond, —O—, —C(═O)—, —NR^{a}—, or —S(═O)₂—.

Preferably, L² is selected from a bond, —O—, —NR^{a}-.

Preferably, L² is selected from —O—, —NR^{a}-.

In one aspect of the present disclosure, wherein, ring A is selected from nitrogen-containing C₃₋₁₀ heteroaryl or C₃₋₁₀ heterocycloalkyl, wherein, the heteroaryl, heterocycloalkyl are optionally substituted with one or more R⁴.

Preferably, ring A is selected from nitrogen-containing C₃₋₁₀ heterocycloalkyl, the C₃₋₁₀ heterocycloalkyl is optionally substituted with one or more R⁴.

In some embodiments, ring A is selected from:

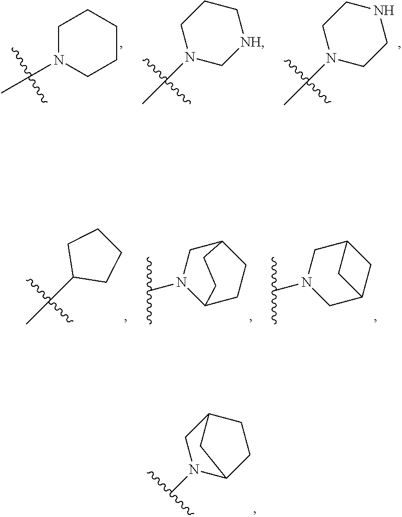

wherein the

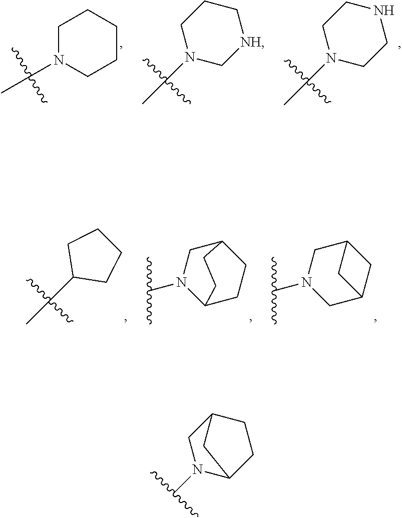

are optionally substituted with R⁴.

Preferably, ring A is selected from:

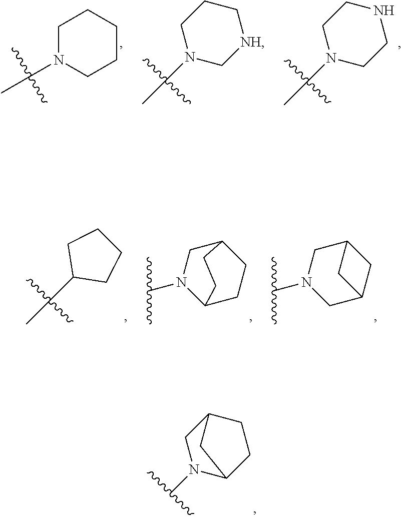

wherein the

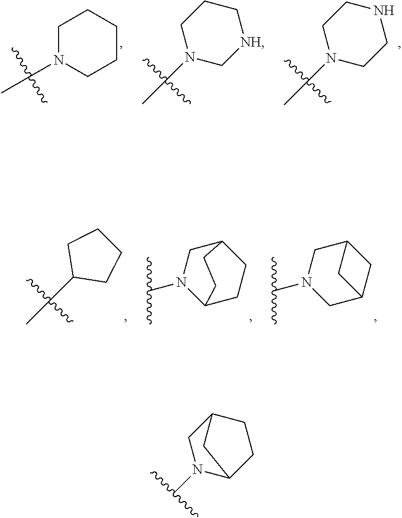

are optionally substituted with R⁴.

In some embodiments, ring A is selected from:

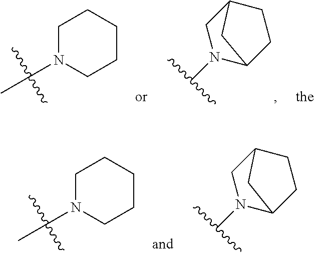 [SMILES: *N1CCCCC1 ; *N1CC2CCC1C2 ; *N1CCCCC1 ; *N1CC2CCC1C2]

are optionally substituted with R⁴.

Preferably, ring A is selected from:

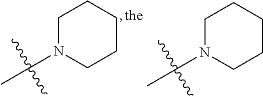 [SMILES: *N1CCCCC1 ; *N1CCCCC1]

is optionally substituted with R⁴.

In one aspect of the present disclosure, wherein,
R⁴ is each independently selected at each occurrence from hydrogen, halogen, CN, hydroxyl, C₁₋₆ alkyl, C₁₋₆ alkoxy, C₂₋₆ alkenyl, —NR^{a}R^{b}.

Preferably, R⁴ is selected from hydrogen, C₁₋₆ alkyl, —NR^{a}R^{b}.

Preferably, R⁴ is selected from hydrogen, —NR^{a}R^{b}.

In one aspect of the present disclosure, wherein,
R¹, R⁶ are each independently selected at each occurrence from hydrogen, halogen, CN, hydroxyl, C₁₋₆ alkyl, C₁₋₆ alkoxy, halogen-substituted C₁₋₆ alkyl, halogen-substituted C₁₋₆ alkoxy, C₃₋₆ cycloalkyl, C₂₋₆ alkenyl, C₂₋₆ alkynyl, COOH, —NR^{a}R^{b}, —C(═O)NR^{a}R^{b}.

Preferably, R¹, R⁶ are each independently selected at each occurrence from hydrogen, CN, hydroxyl, C₁₋₆ alkoxy. Preferably, R¹, R⁶ are each independently selected at each occurrence from hydrogen, CN, hydroxyl.

Preferably, R¹ is each independently selected at each occurrence from hydrogen, C₁₋₆ alkoxy and hydroxyl; R⁶ is each independently selected at each occurrence from hydrogen and CN.

R³ is selected from hydrogen, C₁₋₆ alkyl, C₁₋₆ alkoxy, C₆₋₁₀ aryl or C₆₋₁₀ heteroaryl, wherein, the aryl or heteroaryl is optionally substituted with one or more substituents selected from hydrogen, halogen, CN, CF₃, hydroxyl, C₁₋₆ alkyl, C₁₋₆ alkoxy, C₂₋₆ alkenyl, the heteroaryl, heterocycloalkyl contain 1 to 4 heteroatoms optionally selected from N, O, or S.

Preferably, R³ is selected from hydrogen, C₁₋₆ alkyl, C₆₋₁₀ aryl or C₆₋₁₀ heteroaryl, wherein, the aryl or heteroaryl is optionally substituted with one or more substituents selected from hydrogen, halogen, CN, the heteroaryl, heterocycloalkyl contain 1 to 4 heteroatoms optionally selected from N, O, or S.

Preferably, R³ is selected from hydrogen, C₆₋₁₀ aryl or C₆₋₁₀ heteroaryl, wherein, the aryl or heteroaryl is optionally substituted with one or more substituents selected from hydrogen, halogen, CN, the heteroaryl, heterocycloalkyl contain 1 to 4 heteroatoms optionally selected from N, O, or S.

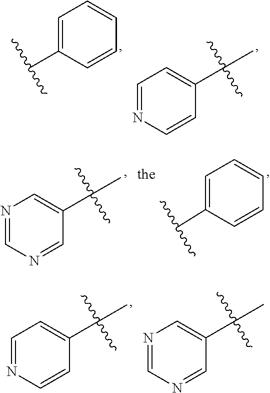

Preferably, R³ is selected from hydrogen, methyl, are optionally substituted with one or more substituents selected from hydrogen, halogen, CN.

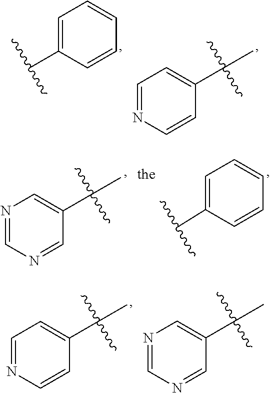

Preferably, R³ is selected from hydrogen, are optionally substituted with one or more substituents selected from hydrogen, halogen, CN.

Preferably, R³ is selected from hydrogen,

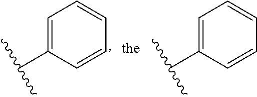 [SMILES: *c1ccccc1 ; *c1ccccc1]

is optionally substituted with one or more substituents selected from hydrogen, halogen, CN.

In one aspect of the present disclosure, wherein,
R⁵ is each independently selected at each occurrence from hydrogen, halogen, CN, hydroxyl, C₁₋₆ alkyl, C₁₋₆ alkoxy, C₂₋₆ alkenyl, C₆₋₁₀ aryl or C₆₋₁₀ heteroaryl, wherein, the alkyl, alkoxy, alkenyl, aryl or heteroaryl is optionally substituted with one or more substituents selected from hydrogen, halogen, CN, CF₃, hydroxyl, C₁₋₆ alkyl, C₁₋₆ alkoxy, C₂₋₆ alkenyl, the heteroaryl, heterocycloalkyl contain 1 to 4 heteroatoms optionally selected from N, O, or S.

Alternatively, R⁵, R⁶ and the atom to which both of them directly connect collectively form cycloalkyl, heteroalicyclic, aryl or heteroaryl, wherein, the cycloalkyl, heteroalicyclic, aryl, heteroaryl are optionally substituted with one or more substituents selected from hydrogen, halogen, CN, hydroxyl, C₁₋₆ alkyl, C₁₋₆ alkoxy, C₂₋₆ alkenyl, C₂₋₆ alkynyl.

Preferably, R⁵ is each independently selected from hydroxyl, C₁₋₆ alkoxy,

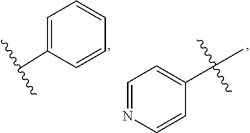

wherein, the alkyl, alkoxy,

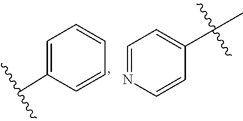

are optionally substituted with one or more substituents selected from hydrogen, halogen, CN, hydroxyl, C₁₋₆ alkyl, C₁₋₆ alkoxy.

Alternatively, R⁵, R⁶ and the atom to which both of them directly connect collectively form

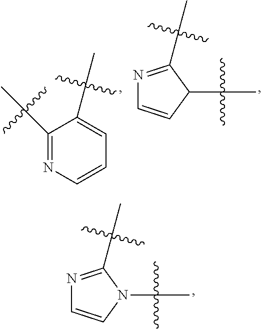

wherein, the

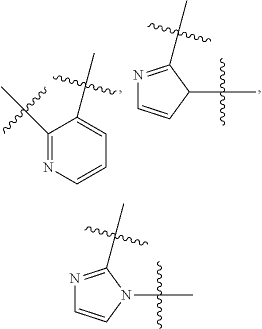

are optionally substituted with one or more of the following groups: hydrogen, halogen, CN, hydroxyl, C₁₋₆ alkyl, C₁₋₆ alkoxy.

Preferably, R⁵ is selected from

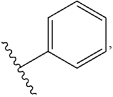

wherein, the

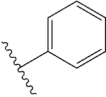 [SMILES: *c1ccccc1]

is optionally substituted with one or more substituents selected from hydrogen, halogen, CN.

Alternatively, R⁵, R⁶ and the atom to which both of them directly connect collectively form

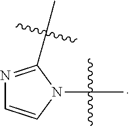 [SMILES: *c1nccn1*]

In one aspect of the present disclosure, wherein,
m is selected from 0, 1, 2 or 3; preferably, m is selected from 1 or 2.

Z is selected from a bond, —CH₂— or —C(═O); preferably, Z is selected from a bond.

In one aspect of the present disclosure, wherein,
R^{a}, R^{b} are each independently selected at each occurrence from hydrogen, halogen, CN, hydroxyl, C₁₋₆ alkyl, C₁₋₆ alkoxy, C₂₋₆ alkenyl, C₂₋₆ alkynyl, COOH, wherein, the alkyl, alkoxy, alkenyl, alkynyl are optionally substituted with one or more substituents selected from hydrogen, halogen, CN, CF₃, hydroxyl, C₁₋₆ alkyl, C₁₋₆ alkoxy, C₂₋₆ alkenyl, C₂₋₆ alkynyl, COOH.

Preferably, R^{a}, R^{b} are each independently selected at each occurrence from hydrogen, C₁₋₆ alkyl;
preferably, R^{a}, R^{b} are each independently selected at each occurrence from hydrogen and methyl.

In some embodiments, the present disclosure provides the compound represented by formula (III-2):

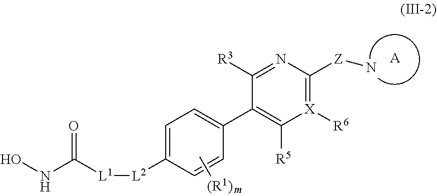

wherein, the definition of each substituent is consistent with the definition of formula (III-1).

The present disclosure provides the compound represented by formula (IV-1a):

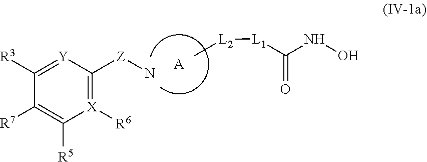

wherein,
L¹ is selected from a bond, —C₁₋₁₀ alkyl-, —C₂₋₆ alkenyl-, —C₁₋₁₀ alkyl-C₂₋₆ alkenyl-, —C₆₋₁₀ heteroaryl-, —C₁₋₁₀ alkyl-(C₆₋₁₀ aryl or heteroaryl or heterocycloalkyl or heterocycloalkenyl)-C₂₋₆ alkenyl-, —(C₆₋₁₀ aryl or heteroaryl)-C₂₋₆ alkenyl-, —C₁₋₁₀ alkyl-(C₆₋₁₀ aryl)-, —C₁₋₁₀ alkyl-(C₆₋₁₀ aryl)-C₁₋₁₀ alkyl-, —NR^{a}—, —C₁₋₁₀ alkyl-(C₆₋₁₀ aryl)-C₂₋₆ alkynyl-, —C₁₋₁₀ alkyl-(C₆₋₁₀ heterocycloalkyl)-(C₆₋₁₀ aryl)-, —C₁₋₁₀ alkyl-NH-6-10-membered heteroaryl-, —C₁₋₁₀ alkyl-6-10-membered heteroaryl-, —C₁₋₁₀ alkyl-C₆₋₁₀ cycloalkenyl-C₂₋₆ alkenyl-, —C₁₋₁₀ alkyl-C₆₋₁₀ aryl-C₃₋₆ cycloalkenyl-, —C₁₋₁₀ alkyl-C₆₋₁₀ aryl-C₃₋₆ cycloalkyl-, —C₁₋₁₀ alkyl-O—C₆₋₁₀ aryl-, —C₁₋₁₀ alkyl-6-10-membered heteroaryl-C₁₋₁₀ alkyl-, —C₁₋₁₀ alkyl-6-10-membered aryl-O—C₁₋₁₀ alkyl-, —C₁₋₁₀ alkyl-6-10-membered heteroaryl-O—C₁₋₁₀ alkyl-, —C₁₋₁₀ alkyl-6-10-membered aryl-S—C₁₋₁₀ alkyl-, the alkyl, alkenyl, cycloalkyl, heterocycloalkyl, heterocycloalkenyl, aryl or heteroaryl is optionally substituted with one or more substituents selected from hydrogen, halogen, CN, CF₃, hydroxyl, C₁₋₆ alkyl, C₁₋₆ alkoxy, C₂₋₆ alkenyl, the heteroaryl, heterocycloalkyl contain 1 to 4 heteroatoms optionally selected from N, O, or S;
preferably, L¹ is selected from a bond, —C₁₋₁₀ alkyl-, —C₂₋₆ alkenyl-, —C₁₋₁₀ alkyl-C₂₋₆ alkenyl-, —C₆₋₁₀ heteroaryl-, —C₁₋₁₀ alkyl-(C₆₋₁₀ aryl or heteroaryl or heterocycloalkyl or heterocycloalkenyl)-C₂₋₆ alkenyl-, —(C₆₋₁₀ aryl or heteroaryl)-C₂₋₆ alkenyl-, —C₁₋₁₀ alkyl-(C₆₋₁₀ aryl)-, —C₁₋₁₀ alkyl-(C₆₋₁₀ aryl)-C₁₋₁₀ alkyl-, —NR^{a}—, —C₁₋₁₀ alkyl-(C₆₋₁₀ aryl)-C₂₋₆ alkynyl-, —C₁₋₁₀ alkyl-(C₆₋₁₀ heterocycloalkyl)-(C₆₋₁₀ aryl)-, —C₁₋₁₀ alkyl-NH-6-10-membered heteroaryl-, —C₁₋₁₀ alkyl-6-10-membered heteroaryl-, —C₁₋₁₀ alkyl-C₆₋₁₀ cycloalkenyl-C₂₋₆ alkenyl-, —C₁₋₁₀ alkyl-C₆₋₁₀ aryl-C₃₋₆ cycloalkenyl-, —C₁₋₁₀ alkyl-C₆₋₁₀ aryl-C₃₋₆ cycloalkyl-, —C₁₋₁₀ alkyl-O—C₆₋₁₀ aryl-, —C₁₋₁₀ alkyl-6-10-membered heteroaryl-O—C₁₋₁₀ alkyl-, the alkyl, alkenyl, cycloalkyl, heterocycloalkyl, heterocycloalkenyl, aryl or heteroaryl is optionally substituted with one or more substituents selected from hydrogen, halogen, CN, CF₃, hydroxyl, C₁₋₆ alkyl, C₁₋₆ alkoxy, C₂₋₆ alkenyl, the heteroaryl, heterocycloalkyl contain 1 to 4 heteroatoms optionally selected from N, O, or S;
alternatively, one or more alkyl groups of the alkyl may optionally be replaced by one or more groups selected from —C(═O)—, —S(═O)₂— or —NR^{a}-.

In some embodiments, in the formula (IV-1a) provided herein, L¹ is selected from a bond, —C₁₋₁₀ alkyl-, —C₂₋₆ alkenyl-, —C₁₋₁₀ alkyl-C₂₋₆ alkenyl-, —C₆₋₁₀ heteroaryl-, —C₁₋₁₀ alkyl-(C₆₋₁₀ aryl or heteroaryl or heterocycloalkyl or heterocycloalkenyl)-C₂₋₆ alkenyl-, —(C₆₋₁₀ aryl or heteroaryl)-C₂₋₆ alkenyl-, —C₁₋₁₀ alkyl-(C₆₋₁₀ aryl)-, —C₁₋₁₀ alkyl-(C₆₋₁₀ aryl)-C₁₋₁₀ alkyl-, —NR^{a}—, —C₁₋₁₀ alkyl-(C₆₋₁₀ aryl)-C₂₋₆ alkynyl-, —C₁₋₁₀ alkyl-(C₆₋₁₀ heterocycloalkyl)-(C₆₋₁₀ aryl)-, the alkyl, alkenyl, cycloalkyl, heterocycloalkyl, heterocycloalkenyl, aryl or heteroaryl is optionally substituted with one or more substituents selected from hydrogen, halogen, CN, CF₃, hydroxyl, C₁₋₆ alkyl, C₁₋₆ alkoxy, C₂₋₆ alkenyl, the heteroaryl, heterocycloalkyl contain 1 to 4 heteroatoms optionally selected from N, O, or S;
alternatively, one or more alkyl groups of the alkyl may optionally be replaced by one or more groups selected from —C(═O)—, —S(═O)₂— or —NR^{a}-.

In some embodiments, in the formula (IV-1a) provided herein, L¹ is selected from —C₁₋₁₀ alkyl-, —C₂₋₆ alkenyl-, —C₆₋₁₀ heteroaryl-, —C₁₋₁₀ alkyl-(C₆₋₁₀ aryl or heteroaryl or heterocycloalkyl or heterocycloalkenyl)-C₂₋₆ alkenyl-, —(C₆₋₁₀ aryl or heteroaryl)-C₂₋₆ alkenyl-, —C₁₋₁₀ alkyl-(C₆₋₁₀ aryl)-, —C₁₋₁₀ alkyl-(C₆₋₁₀ aryl)-C₁₋₁₀ alkyl-, —C₁₋₁₀ alkyl-(C₆₋₁₀ aryl)-C₂₋₆ alkynyl-, —C₁₋₁₀ alkyl-(C₆₋₁₀ heterocycloalkyl)-(C₆₋₁₀ aryl)-, —C₁₋₁₀ alkyl-NH-6-10-membered heteroaryl-, —C₁₋₁₀ alkyl-6-10-membered heteroaryl-, —C₁₋₁₀ alkyl-C₆₋₁₀ cycloalkenyl-C₂₋₆ alkenyl-, —C₁₋₁₀ alkyl-C₆₋₁₀ aryl-C₃₋₆ cycloalkenyl-, —C₁₋₁₀ alkyl-C₆₋₁₀ aryl-C₃₋₆ cycloalkyl-, —C₁₋₁₀ alkyl-O—C₆₋₁₀ aryl-, —C₁₋₁₀ alkyl-6-10-membered heteroaryl-C₁₋₁₀ alkyl-, —C₁₋₁₀ alkyl-6-10-membered aryl-O—C₁₋₁₀ alkyl-, —C₁₋₁₀ alkyl-6-10-membered heteroaryl-O—C₁₋₁₀ alkyl-, —C₁₋₁₀ alkyl-6-10-membered aryl-S—C₁₋₁₀ alkyl-, the alkyl, alkenyl, cycloalkyl, heterocycloalkyl, heterocycloalkenyl, aryl or heteroaryl is optionally substituted with one or more substituents selected from hydrogen, halogen, CN, CF₃, hydroxyl, C₁₋₆ alkyl, C₁₋₆ alkoxy, C₂₋₆ alkenyl, the heteroaryl, heterocycloalkyl contain 1 to 4 heteroatoms optionally selected from N, O, or S.

In some embodiments, in the formula (IV-1a) provided herein, L¹ is selected from —C₁₋₁₀ alkyl-, —C₂₋₆ alkenyl-, —C₆₋₁₀ heteroaryl-, —C₁₋₁₀ alkyl-(C₆₋₁₀ aryl or heteroaryl or heterocycloalkyl or heterocycloalkenyl)-C₂₋₆ alkenyl-, —C₆₋₁₀ aryl-C₂₋₆ alkenyl-, —C₁₋₁₀ alkyl-(C₆₋₁₀ aryl)-, —C₁₋₁₀ alkyl-(C₆₋₁₀ aryl)-C₁₋₁₀ alkyl-, —C₁₋₁₀ alkyl-(C₆₋₁₀ aryl)-C₂₋₆ alkynyl-, —C₁₋₁₀ alkyl-(C₆₋₁₀ heterocycloalkyl)-(C₆₋₁₀ aryl)-, the alkyl, alkenyl, cycloalkyl, heterocycloalkyl, heterocycloalkenyl, aryl or heteroaryl is optionally substituted with one or more substituents selected from hydrogen, halogen, CN, CF₃, hydroxyl, C₁₋₆ alkyl, C₁₋₆ alkoxy, C₂₋₆ alkenyl, the heteroaryl, heterocycloalkyl contain 1 to 4 heteroatoms optionally selected from N, O, or S.

Preferably, L¹ is selected from —C₁₋₁₀ alkyl-, —C₁₋₁₀ alkyl-C₂₋₆ alkenyl-, —C₆₋₁₀ heteroaryl-, —C₁₋₁₀ alkyl-(C₆₋₁₀ aryl or heteroaryl)-C₂₋₆ alkenyl-, —(C₆₋₁₀ aryl or heteroaryl)-C₂₋₆ alkenyl-, —C₁₋₁₀ alkyl-(C₆₋₁₀ aryl)-, —C₁₋₁₀ alkyl-(C₆₋₁₀ aryl)-C₁₋₁₀ alkyl-, —C₁₋₁₀ alkyl-(C₆₋₁₀ heterocycloalkyl)-(C₆₋₁₀ aryl)-, the alkyl, alkenyl, cycloalkyl, heterocycloalkyl, heterocycloalkenyl, aryl or heteroaryl is optionally substituted with one or more substituents selected from hydrogen, halogen, CN, CF₃, hydroxyl, C₁₋₆ alkyl, C₁₋₆ alkoxy, C₂₋₆ alkenyl, the heteroaryl, heterocycloalkyl contain 1 to 4 heteroatoms optionally selected from N, O, or S.

In some embodiments, in the formula (IV-1a) provided herein, L¹ is selected from —C₁₋₁₀ alkyl-, —C₆₋₁₀ heteroaryl-, —C₁₋₁₀ alkyl-(heteroaryl or heterocycloalkyl or heterocycloalkenyl)-C₂₋₆ alkenyl- —C₁₋₁₀ alkyl-(C₆₋₁₀ aryl)-vinyl-, -(6-10-membered heteroaryl)-C₂₋₆ alkenyl-, —C₁₋₁₀ alkyl-(C₆₋₁₀ aryl)-C₁₋₁₀ alkyl-, —C₁₋₁₀ alkyl-(C₆₋₁₀ aryl)-C₂₋₆ alkynyl-, —C₁₋₁₀ alkyl-C₆₋₁₀ cycloalkenyl-C₂₋₆ alkenyl-, —C₁₋₁₀ alkyl-C₆₋₁₀ aryl-C₃₋₆ cycloalkyl-, —C₁₋₁₀ alkyl-O—C₆₋₁₀ aryl-, the alkyl, alkenyl, cycloalkyl, heterocycloalkyl, heterocycloalkenyl, aryl or heteroaryl is optionally substituted with one or more substituents selected from hydrogen, halogen, CN, CF₃, hydroxyl, C₁₋₆ alkyl, C₁₋₆ alkoxy, C₂₋₆ alkenyl, the heteroaryl, heterocycloalkyl contain 1 to 4 heteroatoms optionally selected from N, O, or S.

Preferably, L¹ is selected from —C₁₋₁₀ alkyl-, —C₁₋₁₀ alkyl-C₂₋₆ alkenyl-, —C₆₋₁₀ heteroaryl-, —C₁₋₁₀ alkyl-(C₆₋₁₀ aryl or heteroaryl)-C₂₋₆ alkenyl-, —(C₆₋₁₀ aryl or heteroaryl)-C₂₋₆ alkenyl-, —C₁₋₁₀ alkyl-(C₆₋₁₀ aryl)-, —C₁₋₁₀ alkyl-(C₆₋₁₀ aryl)-C₁₋₁₀ alkyl-, —C₁₋₁₀ alkyl-(C₆₋₁₀ heterocycloalkyl)-(C₆₋₁₀ aryl)-, the alkyl, alkenyl, cycloalkyl, heterocycloalkyl, heterocycloalkenyl, aryl or heteroaryl is optionally substituted with one or more substituents selected from hydrogen, halogen, CN, CF₃, hydroxyl, C₁₋₆ alkyl, C₁₋₆ alkoxy, C₂₋₆ alkenyl, the heteroaryl, heterocycloalkyl contain 1 to 4 heteroatoms optionally selected from N, O, or S.

Preferably, L¹ is selected from —C₁₋₁₀ alkyl-(C₆₋₁₀ aryl or heteroaryl)-C₂₋₆ alkenyl-, the alkyl, aryl or heteroaryl is optionally substituted with one or more substituents selected from hydrogen, halogen, CN, CF₃, hydroxyl, C₁₋₆ alkyl, C₁₋₆ alkoxy.

Preferably, L¹ is selected from —CH₂—, —CH₂—(C═C)—, —(CH₂)₄—, —(CH₂)₆—, —(C═O)-phenyl-(C═C)—, —CH₂-phenyl-,
(CH₂)₃-phenyl-, —CH₂-phenyl-(CH₂)₂—, —(CH₂)₂-phenyl-CH₂—, —CH₂-phenyl-(C═C)—, —(CH₂)₂-phenyl-(C═C)—, —CH₂-phenyl-(C≡C)—, —CH₂-phenyl-(C═C)—CH₂—, -phenyl-(C═C)—, pyrimidinyl,

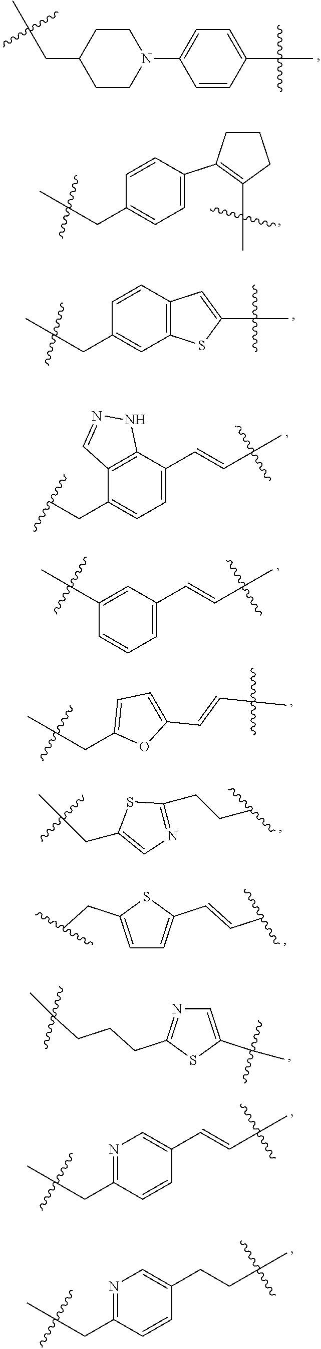

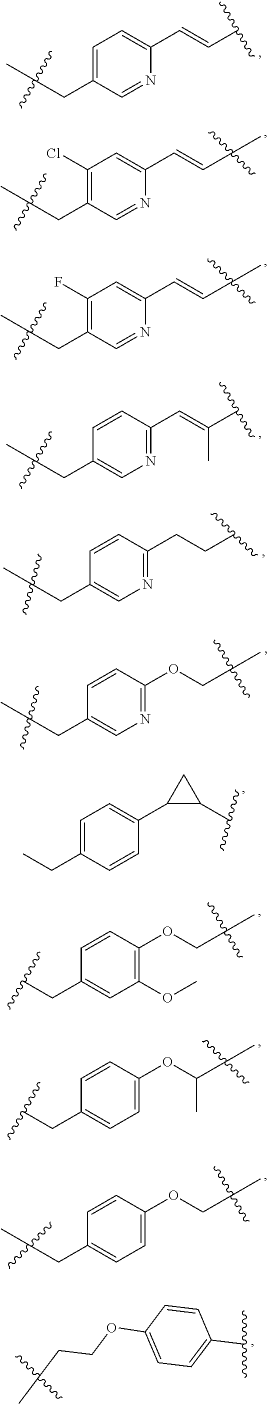

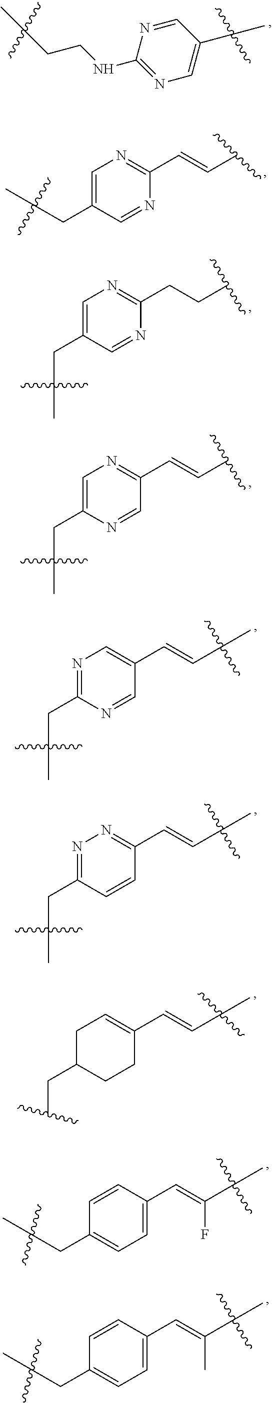

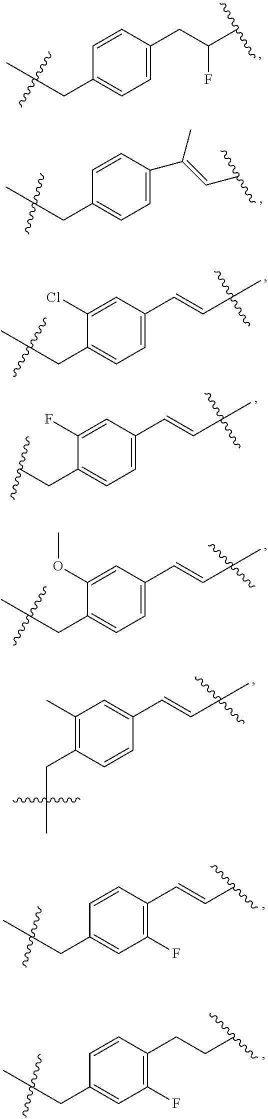

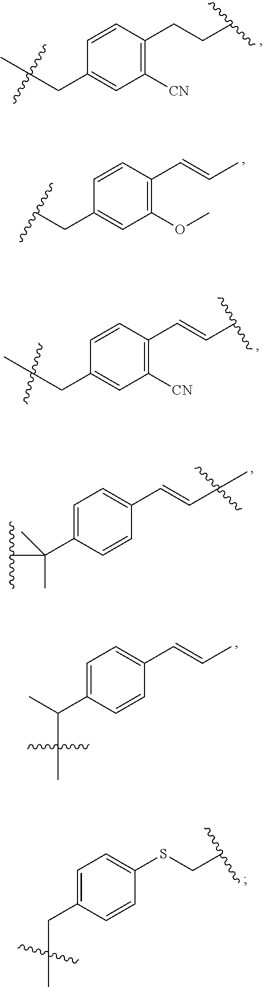

preferably, L¹ is selected from

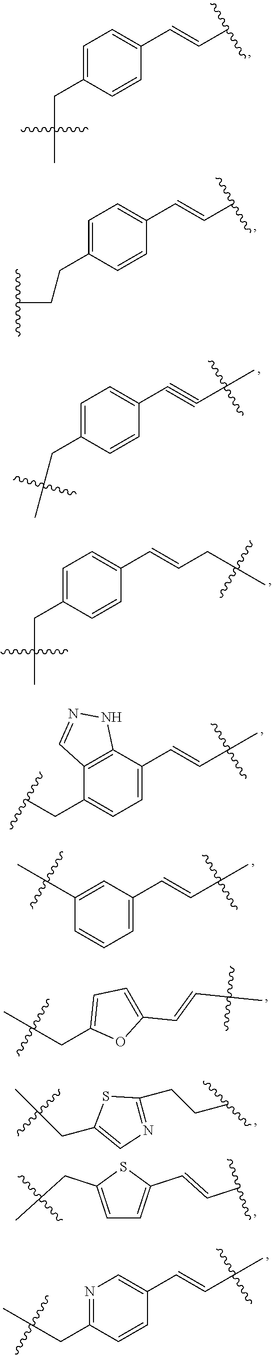

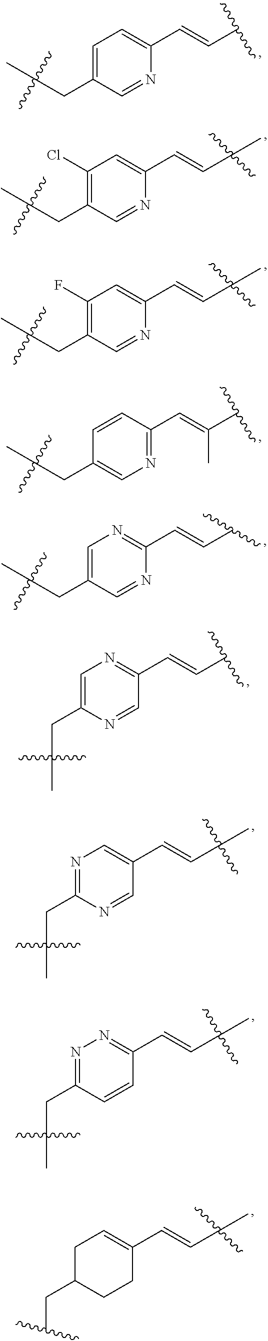

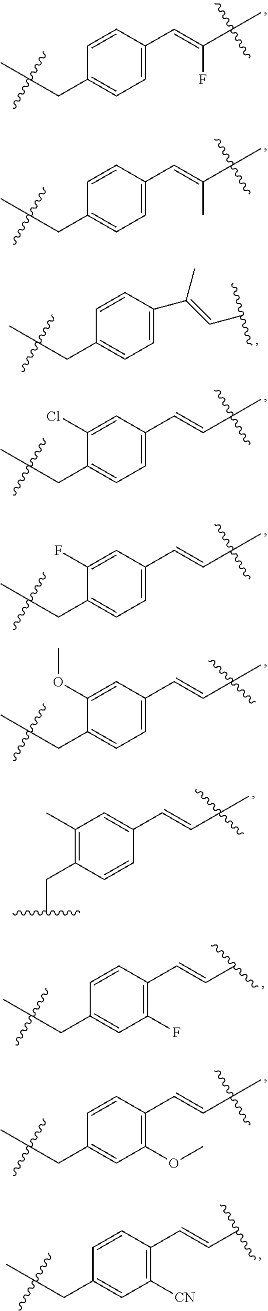

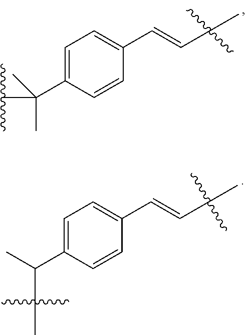 [SMILES: */C=C/c1ccc(C(*)(C)C)cc1 ; */C=C/c1ccc(C(*)C)cc1]

In one aspect of the present disclosure, wherein,
L² is selected from a bond, —O—, —C(═O)—, —S—, —NR^{a}—, —CH₂—NR^{a}—, —NR^{a}—C(═O) and —NR^{a}—S(═O)₂—;
in some embodiments, L² is selected from a bond, —O—, —C(═O)—, —S—, —NR^{a}—, —NR^{a}—C(═O) and —NR^{a}—S(═O)₂—.

In some embodiments, L² is selected from a bond, —O—, —C(═O)—, —S— or —NR^{a}-.

In some embodiments, L² is selected from a bond, —NR^{a}—, —CH₂—NR^{a}—, —NR^{a}—C(═O) and —NR^{a}—S(═O)₂—.

In some embodiments, L² is selected from a bond, —NR^{a}—, —NR^{a}—C(═O) and —NR^{a}—S(═O)₂.

Preferably, L² is selected from a bond, —C(═O)— or —NR^{a}-.

Preferably, L² is selected from —NR^{a}-.

In one aspect of the present disclosure, wherein,
ring A is selected from nitrogen-containing C₃₋₁₀ heteroaryl or C₃₋₁₀ heterocycloalkyl, wherein, the heteroaryl, heterocycloalkyl are optionally substituted with one or more R⁴; the heteroaryl, heterocycloalkyl contain 1 to 4 heteroatoms optionally selected from N, O, or S.

Preferably, ring A is selected from C₃₋₁₀ heterocycloalkyl, wherein, the heterocycloalkyl is optionally substituted with one or more R⁴.

Preferably, ring A is selected from:

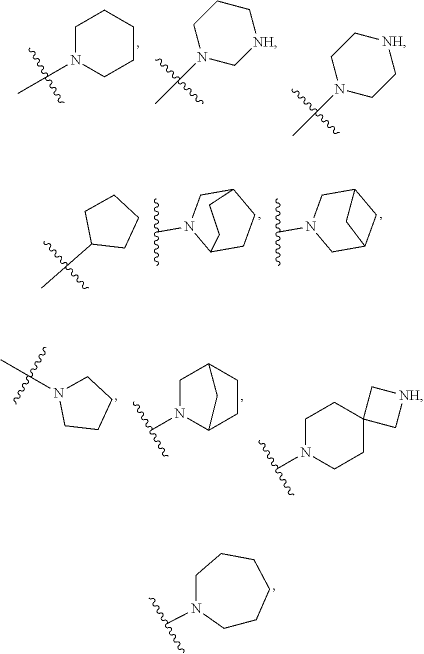

wherein the

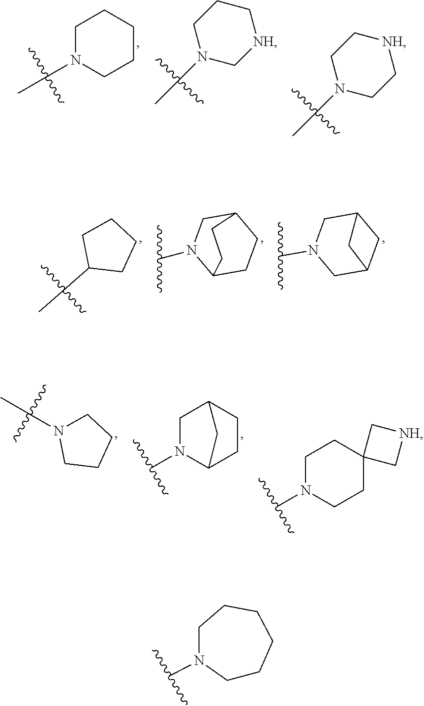

are optionally substituted with R⁴.

In some embodiments, ring A is selected from:

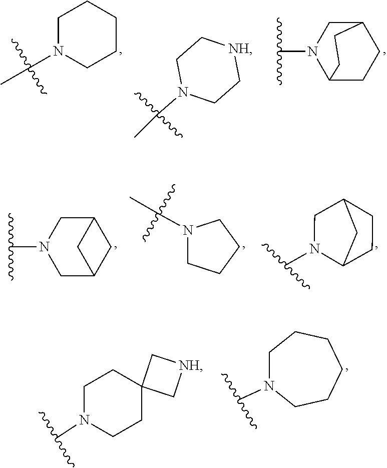

wherein the

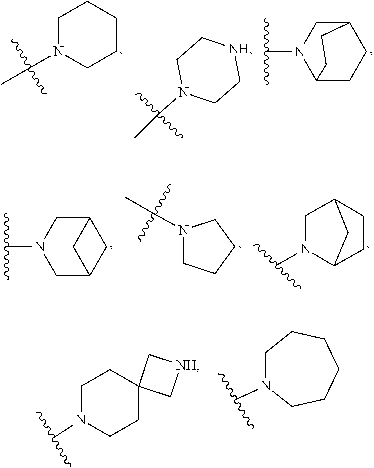

are optionally substituted with R⁴.

In some embodiments, ring A is selected from:

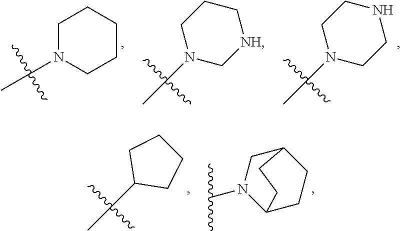

wherein the

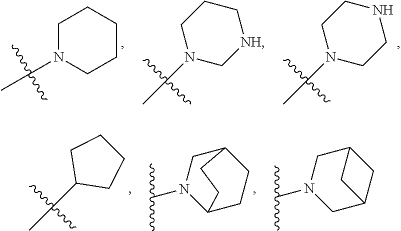

are optionally substituted with R⁴.

In some embodiments, ring A is selected from:

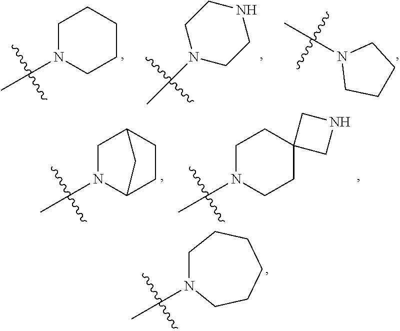

wherein the

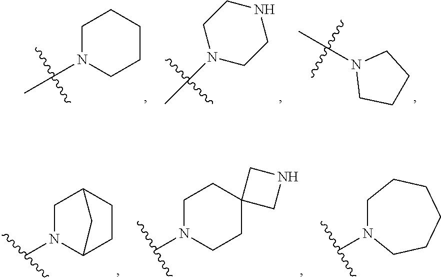

are optionally substituted with R⁴.

Preferably, ring A is selected from:

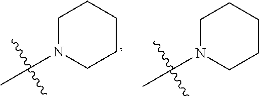 [SMILES: *N1CCCCC1 ; *N1CCCCC1]

is optionally substituted with R⁴.

In one aspect of the present disclosure, wherein,
R⁴ is each independently selected at each occurrence from hydrogen, halogen, CN, hydroxyl, C₁₋₆ alkyl, C₁₋₆ alkoxy, C₂₋₆ alkenyl, —NR^{a}R^{b}.

Preferably, R⁴ is selected from hydrogen, C₁₋₆ alkyl, —NR^{a}R^{b}.

Preferably, R⁴ is selected from hydrogen, C₁₋₆ alkyl.

In one aspect of the present disclosure, wherein,
R⁶ is each independently selected at each occurrence from hydrogen, halogen, CN, hydroxyl, C₁₋₆ alkyl, C₁₋₆ alkyl-CN, halogen-substituted C₁₋₆ alkyl, C₁₋₆ alkoxy, C₃₋₆ cycloalkyl, C₂₋₆ alkenyl, C₂₋₆ alkynyl, COOH, —NR^{a}R^{b}, —C(═O)NR^{a}R^{b}.

Preferably, R⁶ is each independently selected at each occurrence from hydrogen, halogen, CN, hydroxyl, C₁₋₆ alkyl, C₁₋₆ alkoxy, C₃₋₆ cycloalkyl, C₂₋₆ alkenyl, C₂₋₆ alkynyl, COOH, —NR^{a}R^{b}, —C(═O)NR^{a}R^{b}.

Preferably, R⁶ is each independently selected at each occurrence from hydrogen, halogen, CH₂—CN, CN, C₁₋₆ alkyl, C₁₋₆ alkoxy, halogen-substituted C₁₋₆ alkyl.

Preferably, R⁶ is each independently selected at each occurrence from hydrogen, halogen, CN, C₁₋₆ alkyl. Preferably, R⁶ is each independently selected at each occurrence from hydrogen, CN.

R³, R⁷ are each independently selected from hydrogen, C₁₋₆ alkyl, C₁₋₆ alkoxy, C₆₋₁₀ aryl or C₆₋₁₀ heteroaryl, and R³ and R⁷ are not both hydrogen, wherein, the aryl or heteroaryl is optionally substituted with one or more substituents selected from hydrogen, halogen, CN, CF₃, hydroxyl, C₁₋₆ alkyl, C₁₋₆ alkoxy, C₂₋₆ alkenyl, C₂₋₆ alkynyl, C₃₋₆ cycloalkyl, halogen-substituted C₁₋₆ alkoxy, halogen-substituted C₁₋₆ alkyl, hydroxyl-substituted C₁₋₆ alkyl, hydroxyl-substituted C₁₋₆ alkoxy, hydroxyl-substituted C₁₋₆ alkoxy-C₃₋₆ cycloalkyl, COOH, —NR^{a}R^{b}, —S(═O)₂R^{a}, —C(═O)NR^{a}R^{b}, 3 to 6-membered heterocycloalkyl, heterocycloalkenyl, the heteroaryl, heterocycloalkyl contain 1 to 4 heteroatoms optionally selected from N, O, or S.

In some embodiments, R³, R⁷ are each independently selected from hydrogen, C₁₋₆ alkyl, C₁₋₆ alkoxy, C₆₋₁₀ aryl or C₆₋₁₀ heteroaryl, and R³ and R⁷ are not both hydrogen, wherein, the aryl or heteroaryl is optionally substituted with one or more substituents selected from hydrogen, halogen, CN, CF₃, hydroxyl, C₁₋₆ alkyl, C₁₋₆ alkoxy, C₂₋₆ alkenyl, C₂₋₆ alkynyl, C₃₋₆ cycloalkyl, halogen-substituted C₁₋₆ alkoxy, hydroxyl-substituted C₁₋₆ alkyl, hydroxyl-substituted C₁₋₆ alkoxy, COOH, —NR^{a}R^{b}, —S(═O)₂R^{a}, —C(═O)NR^{a}R^{b}, heterocycloalkenyl, the heteroaryl, heterocycloalkyl contain 1 to 4 heteroatoms optionally selected from N, O, or S.

In some embodiments, R³, R⁷ are each independently selected from hydrogen, C₁₋₆ alkyl, C₁₋₆ alkoxy, C₆₋₁₀ aryl or C₆₋₁₀ heteroaryl, and R³ and R⁷ are not both hydrogen, wherein, the aryl or heteroaryl is optionally substituted with one or more substituents selected from hydrogen, halogen, CN, CF₃, hydroxyl, C₁₋₆ alkyl, C₁₋₆ alkoxy, C₂₋₆ alkenyl, C₂₋₆ alkynyl, halogen-substituted C₁₋₆ alkoxy, halogen-substituted C₁₋₆ alkyl, hydroxyl-substituted C₁₋₆ alkoxy, hydroxyl-substituted C₁₋₆ alkoxy-C₃₋₆ cycloalkyl, COOH, —NR^{a}R^{b}, —S(═O)₂R^{a}, —C(═O)NR^{a}R^{b}, 3 to 6-membered heterocycloalkyl, heterocycloalkenyl, the heteroaryl, heterocycloalkyl contain 1 to 4 heteroatoms optionally selected from N, O, or S.

Preferably, R³, R⁷ are each independently selected from hydrogen, C₁₋₆ alkyl, C₁₋₆ alkoxy, C₆₋₁₀ aryl or C₆₋₁₀ heteroaryl, and R³ and R⁷ are not both hydrogen, wherein, the aryl or heteroaryl is optionally substituted with one or more substituents selected from hydrogen, halogen, CN, hydroxyl, C₁₋₆ alkyl, C₁₋₆ alkoxy, heterocycloalkenyl, the heteroaryl, heterocycloalkyl contain 1 to 4 heteroatoms optionally selected from N, O, or S.

Preferably, R³, R⁷ are selected from hydrogen,

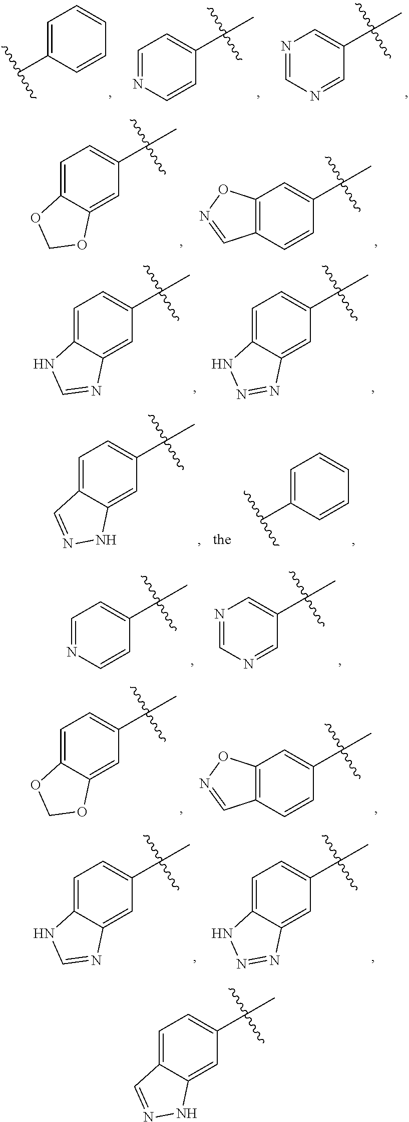

are optionally substituted with one or more substituents selected from hydrogen, halogen, CN, heterocycloalkenyl, hydroxyl, CF₃, C₁₋₆ alkyl, C₁₋₆ alkoxy, C₃₋₆ cycloalkyl, halogen-substituted C₁₋₆ alkoxy, 3 to 6-membered heterocycloalkyl, hydroxyl-substituted C₁₋₆ alkoxy-C₃₋₆ cycloalkyl, hydroxyl-substituted C₁₋₆ alkyl, hydroxyl-substituted C₁₋₆ alkoxy, —NH₂, —N(C₁₋₆ alkyl)₂, —NH(C₁₋₆ alkyl).

Preferably, R³, R⁷ are selected from hydrogen,

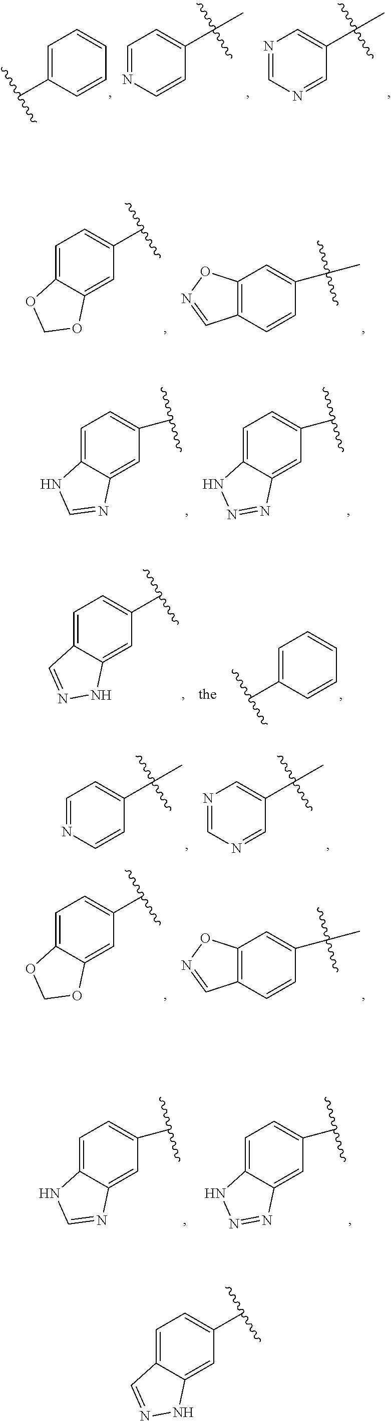

are optionally substituted with one or more substituents selected from hydrogen, halogen, CN, heterocycloalkenyl, hydroxyl, CF₃, C₁₋₆ alkyl, C₁₋₆ alkoxy, C₃₋₆ cycloalkyl, halogen-substituted C₁₋₆ alkoxy, hydroxyl-substituted C₁₋₆ alkyl, hydroxyl-substituted C₁₋₆ alkoxy.

In some embodiments, R³, R⁷ are selected from hydrogen,

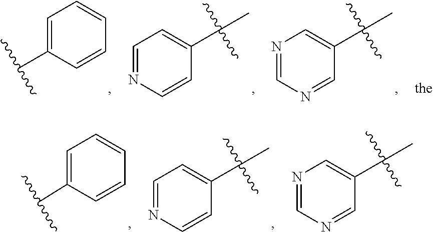

are optionally substituted with one or more substituents selected from hydrogen, halogen, heterocycloalkenyl, hydroxyl.

In some embodiments, R³, R⁷ are selected from hydrogen,

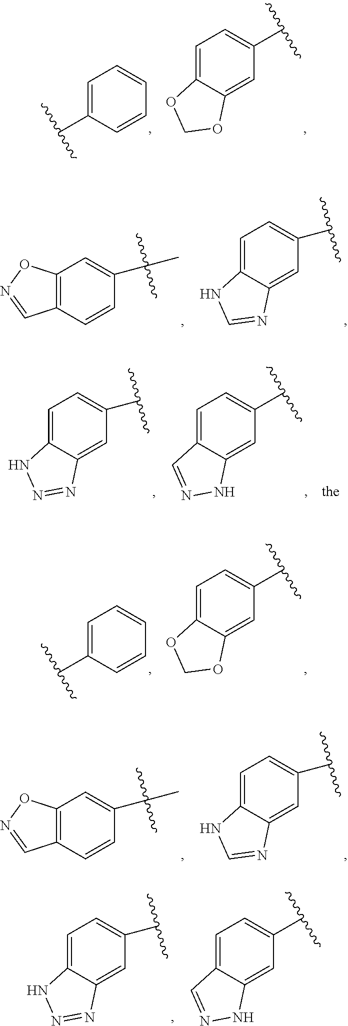

are optionally substituted with one or more substituents selected from hydrogen, halogen, CN, hydroxyl, CF₃, C₁₋₆ alkyl, C₁₋₆ alkoxy, C₃₋₆ cycloalkyl, halogen-substituted C₁₋₆ alkoxy, 3 to 6-membered heterocycloalkyl, hydroxyl-substituted C₁₋₆ alkoxy-C₃₋₆ cycloalkyl, hydroxyl-substituted C₁₋₆ alkyl, hydroxyl-substituted C₁₋₆ alkoxy, —NH₂, —N(C₁₋₆ alkyl)₂, —NH(C₁₋₆ alkyl).

Preferably, R³, R⁷ are selected from hydrogen,

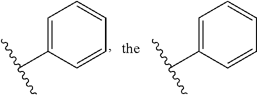 [SMILES: *c1ccccc1 ; *c1ccccc1]

is optionally substituted with one or more substituents selected from hydrogen, halogen, hydroxyl, CN, CF₃, C₁₋₆ alkyl, C₁₋₆ alkoxy, C₃₋₆ cycloalkyl, halogen-substituted C₁₋₆ alkoxy, 3 to 6-membered heterocycloalkyl, hydroxyl-substituted C₁₋₆ alkoxy-C₃₋₆ cycloalkyl, hydroxyl-substituted C₁₋₆ alkyl, hydroxyl-substituted C₁₋₆ alkoxy, —NH₂, —N(C₁₋₆ alkyl)₂, —NH(C₁₋₆ alkyl).

Preferably, R³, R⁷ are selected from hydrogen,

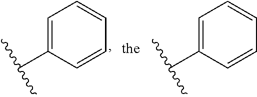 [SMILES: *c1ccccc1 ; *c1ccccc1]

is optionally substituted with one or more substituents selected from hydrogen, halogen, heterocycloalkenyl, hydroxyl.

In one aspect of the present disclosure, wherein,
R⁵ is each independently selected at each occurrence from hydrogen, halogen, CN, hydroxyl, C₁₋₆ alkyl, C₁₋₆ alkoxy, ═O, C₂₋₆ alkenyl, C₆₋₁₀ aryl or C₆₋₁₀ heteroaryl, wherein, the alkyl, alkoxy, alkenyl, aryl or heteroaryl is optionally substituted with one or more substituents selected from hydrogen, halogen, CN, NO₂, CF₃, CHF₂, hydroxyl, C₁₋₆ alkyl, C₁₋₆ alkoxy, —C(═O)—C₁₋₆ alkoxy, C₂₋₆ alkenyl, —C(═O)—NH₂, the heteroaryl, heterocycloalkyl contain 1 to 4 heteroatoms optionally selected from N, O, or S.

In some embodiments, R⁵ is each independently selected at each occurrence from hydrogen, halogen, CN, hydroxyl, C₁₋₆ alkyl, C₁₋₆ alkoxy, ═O, C₂₋₆ alkenyl, C₆₋₁₀ aryl or C₆₋₁₀ heteroaryl, wherein, the alkyl, alkoxy, alkenyl, aryl or heteroaryl is optionally substituted with one or more substituents selected from hydrogen, halogen, CN, NO₂, CF₃, hydroxyl, C₁₋₆ alkyl, C₁₋₆ alkoxy, C₂₋₆ alkenyl, the heteroaryl, heterocycloalkyl contain 1 to 4 heteroatoms optionally selected from N, O, or S.

In some embodiments, R⁵ is each independently selected at each occurrence from hydrogen, halogen, CN, hydroxyl, C₁₋₆ alkyl, C₁₋₆ alkoxy, ═O, C₂₋₆ alkenyl, C₆₋₁₀ aryl or C₆₋₁₀ heteroaryl, wherein, the alkyl, alkoxy, alkenyl, aryl or heteroaryl is optionally substituted with one or more substituents selected from hydrogen, halogen, CN, NO₂, CF₃, hydroxyl, C₁₋₆ alkyl, C₁₋₆ alkoxy, —C(═O)—C₁₋₆ alkoxy, C₂₋₆ alkenyl, the heteroaryl, heterocycloalkyl contain 1 to 4 heteroatoms optionally selected from N, O, or S.

In some embodiments, R⁵ is each independently selected at each occurrence from hydrogen, CN, C₁₋₆ alkoxy, ═O, C₆₋₁₀ aryl or C₆₋₁₀ heteroaryl, wherein, the alkoxy, aryl or heteroaryl is optionally substituted with one or more substituents selected from hydrogen, halogen, CN, NO₂, CF₃, CHF₂, hydroxyl, C₁₋₆ alkyl, —C(═O)—C₁₋₆ alkoxy, —C(═O)—NH₂, the heteroaryl, heterocycloalkyl contain 1 to 4 heteroatoms optionally selected from N, O, or S;
alternatively, R⁵, R⁶ and the atom to which both of them directly connect collectively form cycloalkyl, heteroalicyclic, aryl or heteroaryl, wherein, the cycloalkyl, heteroalicyclic, aryl, heteroaryl are optionally substituted with one or more substituents selected from hydrogen, halogen, CN, hydroxyl, C₁₋₆ alkyl, C₁₋₆ alkoxy, C₂₋₆ alkenyl, C₂₋₆ alkynyl.

Preferably, R⁵ is each independently selected from CN, C₁₋₆ alkoxy, ═O,

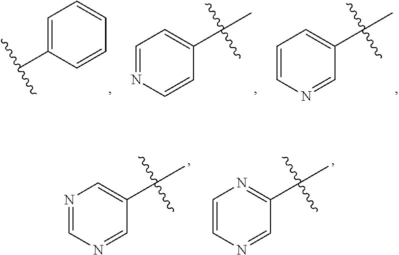

wherein, the alkoxy,

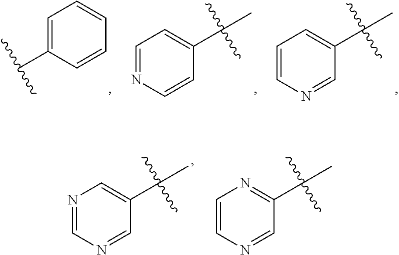

are optionally substituted with one or more substituents selected from hydrogen, halogen, CN, NO₂, CF₃, CHF₂, hydroxyl, C₁₋₆ alkyl, —C(O)OCH₃, —C(═O)—NH₂.

Preferably, R⁵ is each independently selected from

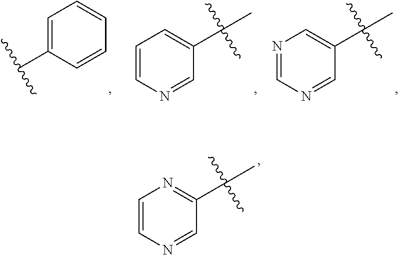

wherein, the

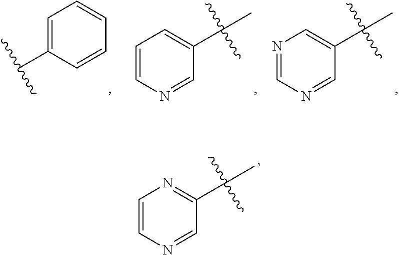

are optionally substituted with one or more substituents selected from hydrogen, halogen, CN, NO₂, CF₃, CHF₂, hydroxyl, C₁₋₆ alkyl, —C(O)OCH₃, —C(═O)—NH₂.

Preferably, R⁵ is each independently selected from CN, C₁₋₆ alkoxy, ═O,

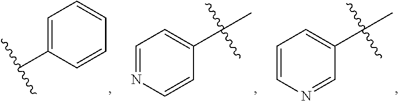

wherein, the alkoxy,

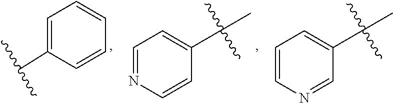

are optionally substituted with one or more substituents selected from hydrogen, halogen, CN, NO₂, hydroxyl.

In some embodiments, R⁵ is each independently selected from C₁₋₆ alkoxy, ═O,

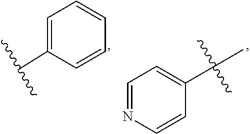

wherein, the alkoxy,

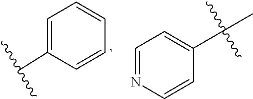

are optionally substituted with one or more substituents selected from hydrogen, halogen, CN, hydroxyl.

Alternatively, R⁵, R⁶ and the atom to which both of them directly connect collectively form

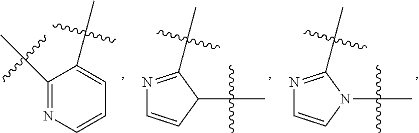

wherein, the

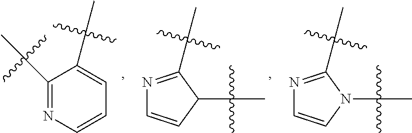

are optionally substituted with one or more of the following groups: hydrogen, halogen, CN, hydroxyl, C₁₋₆ alkyl, C₁₋₆ alkoxy.

Preferably, R⁵ is selected from C₁₋₆ alkoxy,

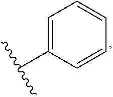

wherein, the

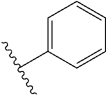 [SMILES: *c1ccccc1]

is optionally substituted with one or more substituents selected from hydrogen, halogen, CN, NO₂.

In some embodiments, R⁵ is selected from C₁₋₆ alkoxy,

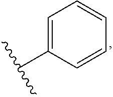

wherein, the

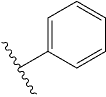 [SMILES: *c1ccccc1]

is optionally substituted with one or more substituents selected from hydrogen, halogen, CN.

Alternatively, R⁵, R⁶ and the atom to which both of them directly connect collectively form

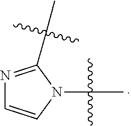 [SMILES: *c1nccn1*]

In one aspect of the present disclosure, wherein,
X, Y are each independently selected from C, N.

Z is selected from a bond, —CH₂—, —C(═O) or —S(═O)₂—.

Preferably, Z is selected from a bond, —CH₂— or —C(═O).

Preferably, Z is selected from a bond.

In one aspect of the present disclosure, wherein,
R^{a}, R^{b} are each independently selected at each occurrence from hydrogen, hydroxyl, C₁₋₆ alkyl, C₁₋₆ alkoxy, C₂₋₆ alkenyl, C₂₋₆ alkynyl, C₃₋₆ cycloalkyl, halogen-substituted C₁₋₆ alkyl, the heteroaryl, heterocycloalkyl contain 1 to 4 heteroatoms optionally selected from N, O, or S.

Preferably, R^{a}, R^{b} are each independently selected at each occurrence from hydrogen, C₁₋₆ alkyl.

Preferably, R^{a}, R^{b} are each independently selected at each occurrence from hydrogen, methyl, ethyl, n-propyl, isopropyl.

Preferably, R^{a}, R^{b} are each independently selected at each occurrence from hydrogen.

In some embodiments, the present disclosure provides the compound represented by formula (IV-2a), formula (IV-3a), formula (IV-4a):

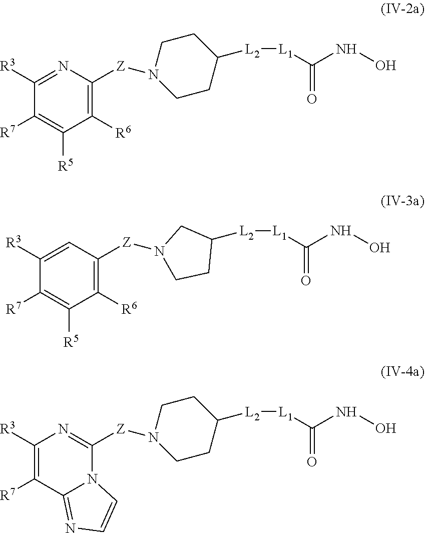

wherein, the definition of each substituent is consistent with the definition in formula (IV-1a).

The present disclosure provides the compound represented by formula (V):

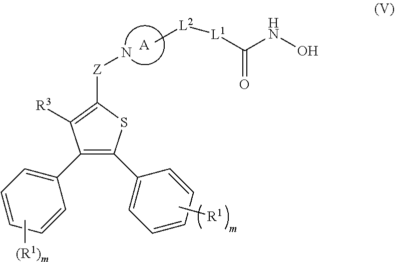

wherein,
L¹ is selected from a bond, —C₁₋₁₀ alkyl-, —C₂₋₆ alkenyl-, —C₁₋₁₀ alkyl-(C₆₋₁₀ aryl or heteroaryl)-C₂₋₆ alkenyl-, —NR^{a}—, the alkyl, alkoxy, alkenyl, alkynyl, aryl or heteroaryl is optionally substituted with one or more substituents selected from hydrogen, halogen, CN, CF₃, hydroxyl, C₁₋₆ alkyl, C₁₋₆ alkoxy, C₂₋₆ alkenyl.

Preferably, L¹ is selected from —C₁₋₁₀ alkyl-(C₆₋₁₀ aryl or heteroaryl)-C₂₋₆ alkenyl-, the aryl or heteroaryl is optionally substituted with one or more substituents selected from hydrogen, halogen, CN, CF₃, hydroxyl, C₁₋₆ alkyl, C₁₋₆ alkoxy, C₂₋₆ alkenyl.

Preferably, L¹ is selected from —C₁₋₁₀ alkyl-(C₆₋₁₀ aryl)-C₂₋₆ alkenyl-, the aryl or heteroaryl is optionally substituted with one or more substituents selected from hydrogen, halogen, CN, CF₃, hydroxyl, C₁₋₆ alkyl, C₁₋₆ alkoxy, C₂₋₆ alkenyl.

In one aspect of the present disclosure, wherein,
L² is selected from a bond or —NR^{a}—.

Preferably, L² is selected from —NR^{a}-.

In one aspect of the present disclosure, wherein,
ring A is selected from nitrogen-containing C₃₋₁₀ heteroaryl or C₃₋₁₀ heterocycloalkyl, wherein, the heteroaryl, heterocycloalky are optionally substituted with one or more R⁴.

Preferably, ring A is selected from C₃₋₁₀ heterocycloalkyl, wherein, the heterocycloalkyl is optionally substituted with one or more R⁴.

Preferably, ring A is selected from:

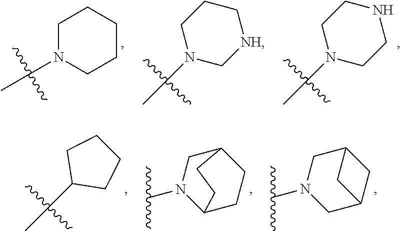

wherein the

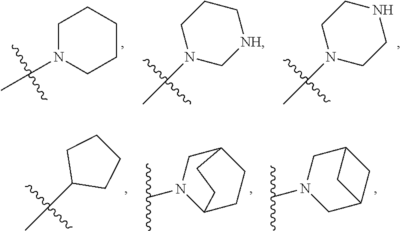

are optionally substituted with one or more R⁴.

Preferably, ring A is selected from:

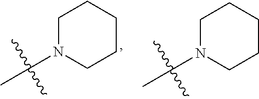 [SMILES: *N1CCCCC1 ; *N1CCCCC1]

are optionally substituted with one or more R⁴.

In one aspect of the present disclosure, wherein,
R⁴ is each independently selected at each occurrence from hydrogen, halogen, CN, hydroxyl, C₁₋₆ alkyl, wherein, the alkyl, alkoxy are optionally substituted with one or more substituents selected from hydrogen, halogen, CN, CF₃, hydroxyl, C₁₋₆ alkyl, C₁₋₆ alkoxy.

Preferably, R⁴ is selected from hydrogen.

In one aspect of the present disclosure, wherein,
R¹ is each independently selected at each occurrence from hydrogen, halogen, CN, hydroxyl, C₁₋₆ alkyl, C₁₋₆ alkoxy, C₂₋₆ alkenyl, —NR^{a}R^{b}, C₆₋₁₀ aryl or C₆₋₁₀ heteroaryl.

Preferably, R¹ is each independently selected at each occurrence from hydrogen, halogen, CN, hydroxyl, C₁₋₆ alkoxy.

R³ is each independently selected at each occurrence from hydrogen, halogen, CN, hydroxyl, C₁₋₆ alkyl, C₁₋₆ alkoxy, C₂₋₆ alkenyl.

Preferably, R³ is each independently selected at each occurrence from hydrogen.

In one aspect of the present disclosure, wherein,
Z is selected from a bond, —CH₂— or —C(═O).

Preferably, Z is selected from —C(═O).

In one aspect of the present disclosure, wherein,
R^{a}, R^{b} are each independently selected at each occurrence from hydrogen, halogen, CN, hydroxyl, C₁₋₆ alkyl, C₁₋₆ alkoxy, wherein, the alkyl, alkoxy are optionally substituted with one or more substituents selected from hydrogen, halogen, CN, CF₃, hydroxyl.

Preferably, R^{a}, R^{b} are each independently selected at each occurrence from hydrogen.

The present disclosure provides the compound represented by formula (VI):

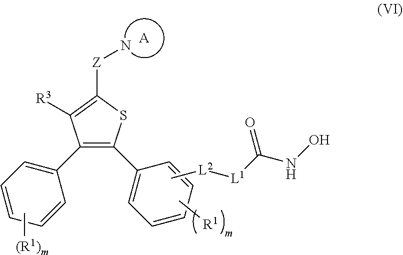

wherein,
L′ is selected from a bond, —C₁₋₁₀ alkyl-, —C₂₋₆ alkenyl-, —NR^{a}—, the alkyl, alkoxy, alkenyl, alkynyl, aryl or heteroaryl is optionally substituted with one or more substituents selected from hydrogen, halogen, CN, CF₃, hydroxyl, C₁₋₆ alkyl, C₁₋₆ alkoxy, C₂₋₆ alkenyl.

Preferably, L¹ is selected from —C₁₋₁₀ alkyl-.

In one aspect of the present disclosure, wherein,
L² is selected from a bond, —O— or —NR^{a}—.

Preferably, L² is selected from —O—.

In one aspect of the present disclosure, wherein,
ring A is selected from nitrogen-containing C₃₋₁₀ heteroaryl or C₃₋₁₀ heterocycloalkyl, wherein, the heteroaryl, heterocycloalkyl are optionally substituted with one or more R⁴.

Preferably, ring A is selected from C₃₋₁₀ heterocycloalkyl, wherein, the heterocycloalkyl is optionally substituted with one or more R⁴.

Preferably, ring A is selected from:

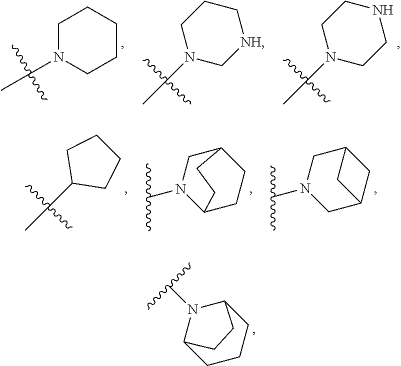

wherein the

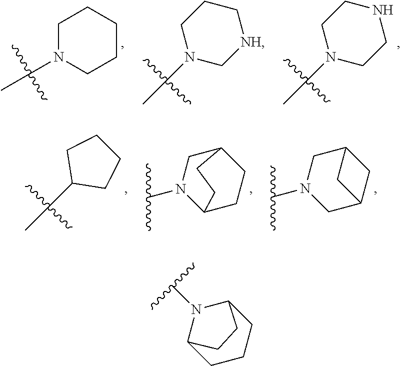

are optionally substituted with R⁴.

In some embodiments, ring A is selected from:

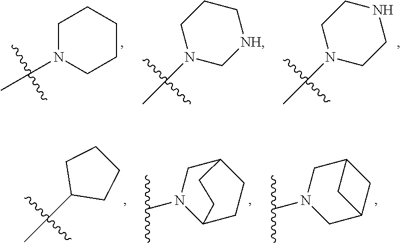

wherein the

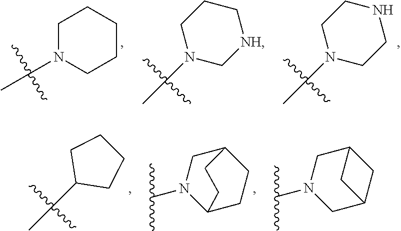

are optionally substituted with R⁴.

Preferably, ring A is selected from:

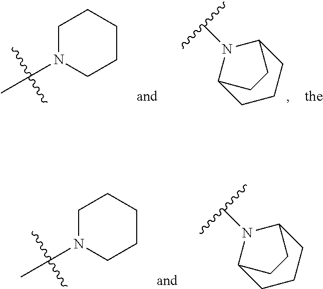 [SMILES: *N1CCCCC1 ; *N1C2CCCC1CC2 ; *N1CCCCC1 ; *N1C2CCCC1CC2]

are optionally substituted with R⁴.

Preferably, ring A is selected from:

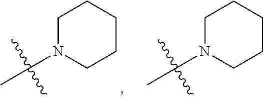 [SMILES: *N1CCCCC1 ; *N1CCCCC1]

are optionally substituted with R⁴.

In one aspect of the present disclosure, wherein,
R⁴ is each independently selected at each occurrence from hydrogen, halogen, CN, hydroxyl, C₁₋₆ alkyl, —NR^{a}R^{b}, wherein, the alkyl, alkoxy are optionally substituted with one or more substituents selected from hydrogen, halogen, CN, CF₃, hydroxyl, C₁₋₆ alkyl, C₁₋₆ alkoxy.

Preferably, R⁴ is selected from hydrogen, C₁₋₆ alkyl, —NR^{a}R^{b}.

In one aspect of the present disclosure, wherein,
R¹ is each independently selected at each occurrence from hydrogen, halogen, CN, hydroxyl, C₁₋₆ alkyl, C₁₋₆ alkoxy, C₂₋₆ alkenyl, —NR^{a}R^{b}.

Preferably, R¹ is each independently selected at each occurrence from hydrogen, halogen, CN, hydroxyl.

R³ is each independently selected at each occurrence from hydrogen, halogen, CN, hydroxyl, C₁₋₆ alkyl, C₁₋₆ alkoxy, C₂₋₆ alkenyl, —NR^{a}R^{b}.

Preferably, R³ is each independently selected at each occurrence from hydrogen.

In one aspect of the present disclosure, wherein,
Z is selected from a bond, —CH₂— or —C(═O).

Preferably, Z is selected from —C(═O).

In one aspect of the present disclosure, wherein,
R^{a}, R^{b} are each independently selected at each occurrence from hydrogen, halogen, CN, hydroxyl, C₁₋₆ alkyl, C₁₋₆ alkoxy, wherein, the alkyl, alkoxy are optionally substituted with one or more substituents selected from hydrogen, halogen, CN, CF₃, hydroxyl.

Preferably, R^{a}, R^{b} are each independently selected at each occurrence from hydrogen.

The present disclosure provides the following compounds:

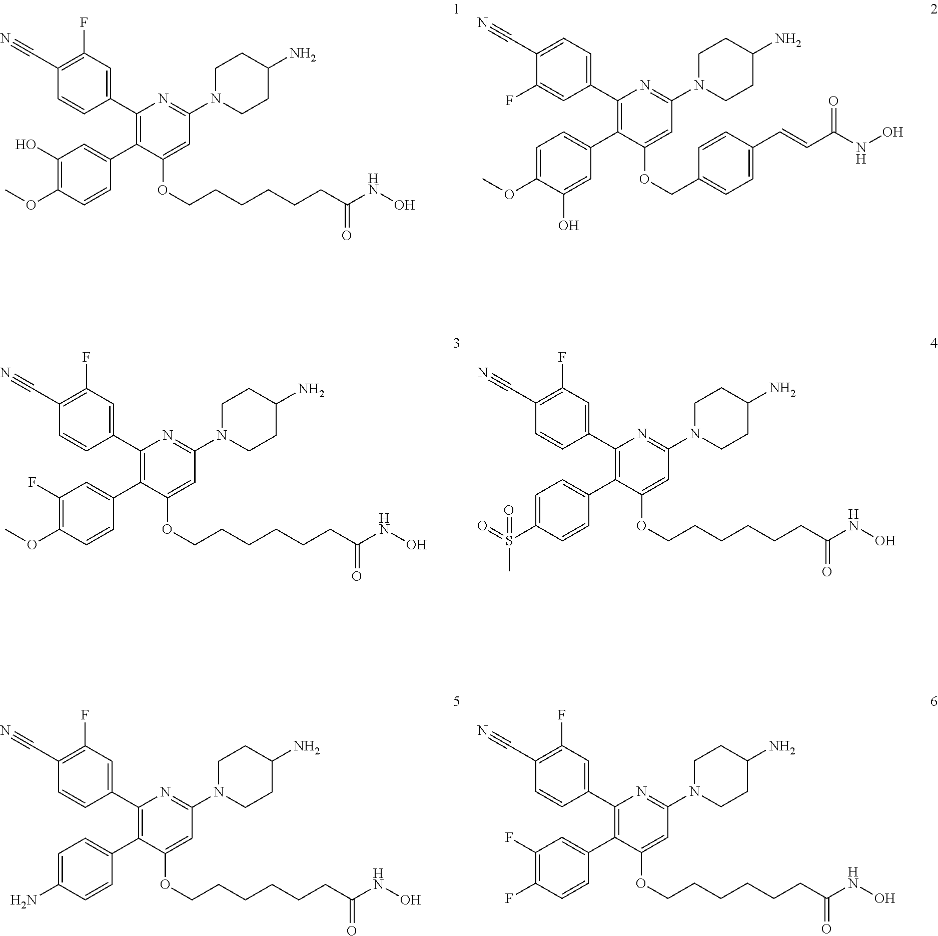 [SMILES: COc1ccc(-c2c(OCCCCCCC(=O)NO)cc(N3CCC(N)CC3)nc2-c2ccc(C#N)c(F)c2)cc1O ; COc1ccc(-c2c(OCc3ccc(/C=C/C(=O)NO)cc3)cc(N3CCC(N)CC3)nc2-c2ccc(C#N)c(F)c2)cc1O ; COc1ccc(-c2c(OCCCCCCC(=O)NO)cc(N3CCC(N)CC3)nc2-c2ccc(C#N)c(F)c2)cc1F ; CS(=O)(=O)c1ccc(-c2c(OCCCCCCC(=O)NO)cc(N3CCC(N)CC3)nc2-c2ccc(C#N)c(F)c2)cc1 ; N#Cc1ccc(-c2nc(N3CCC(N)CC3)cc(OCCCCCCC(=O)NO)c2-c2ccc(N)cc2)cc1F ; N#Cc1ccc(-c2nc(N3CCC(N)CC3)cc(OCCCCCCC(=O)NO)c2-c2ccc(F)c(F)c2)cc1F]

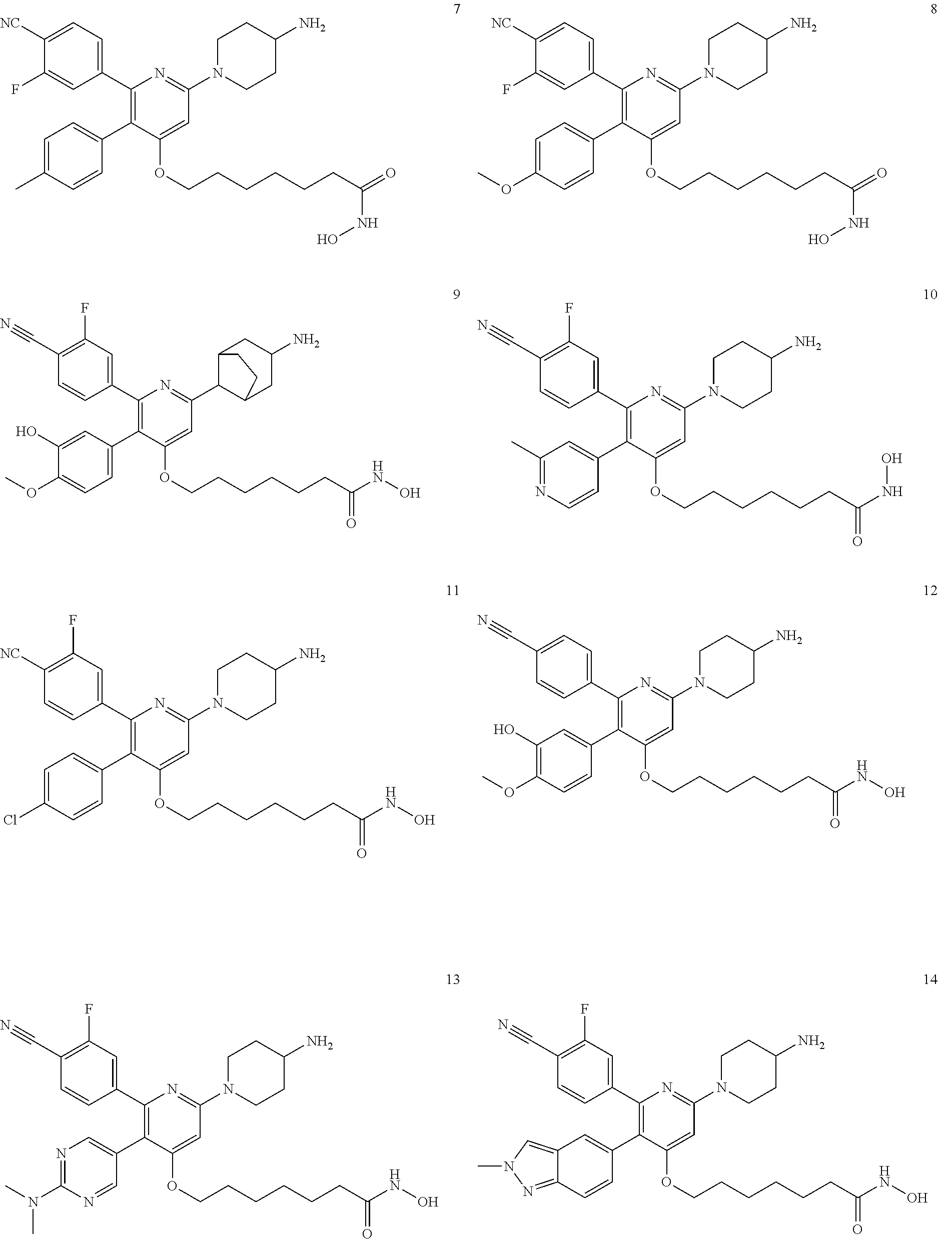

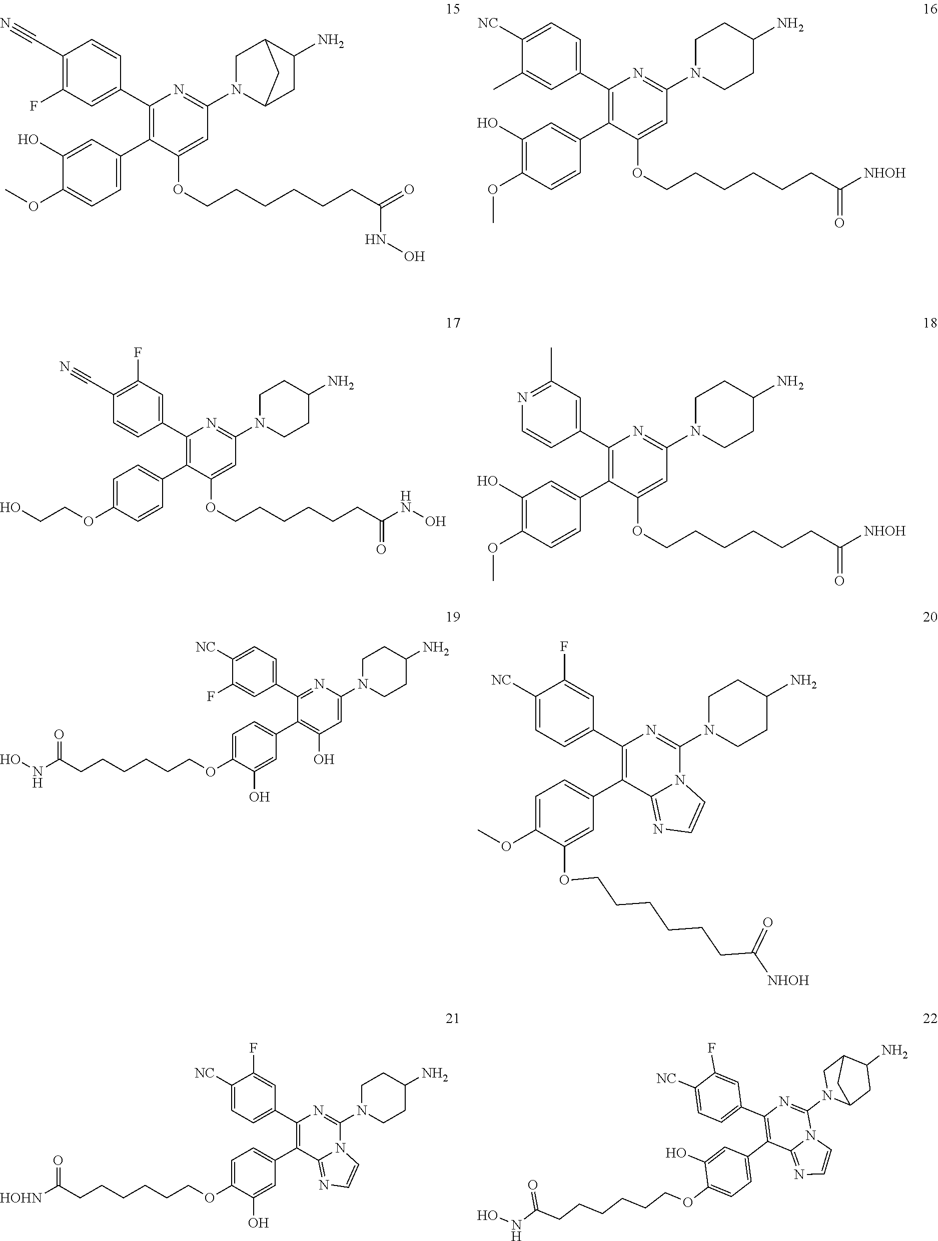

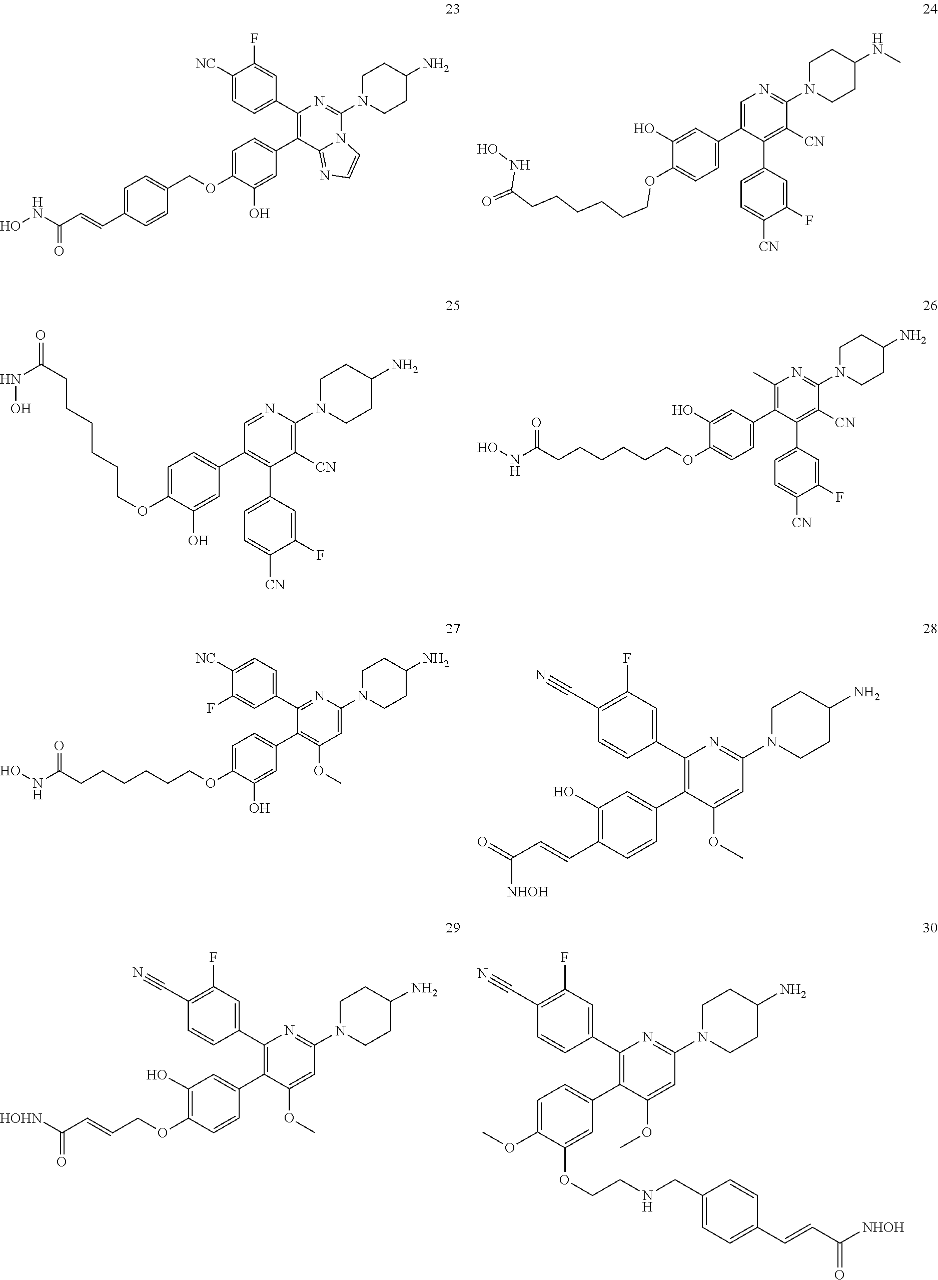

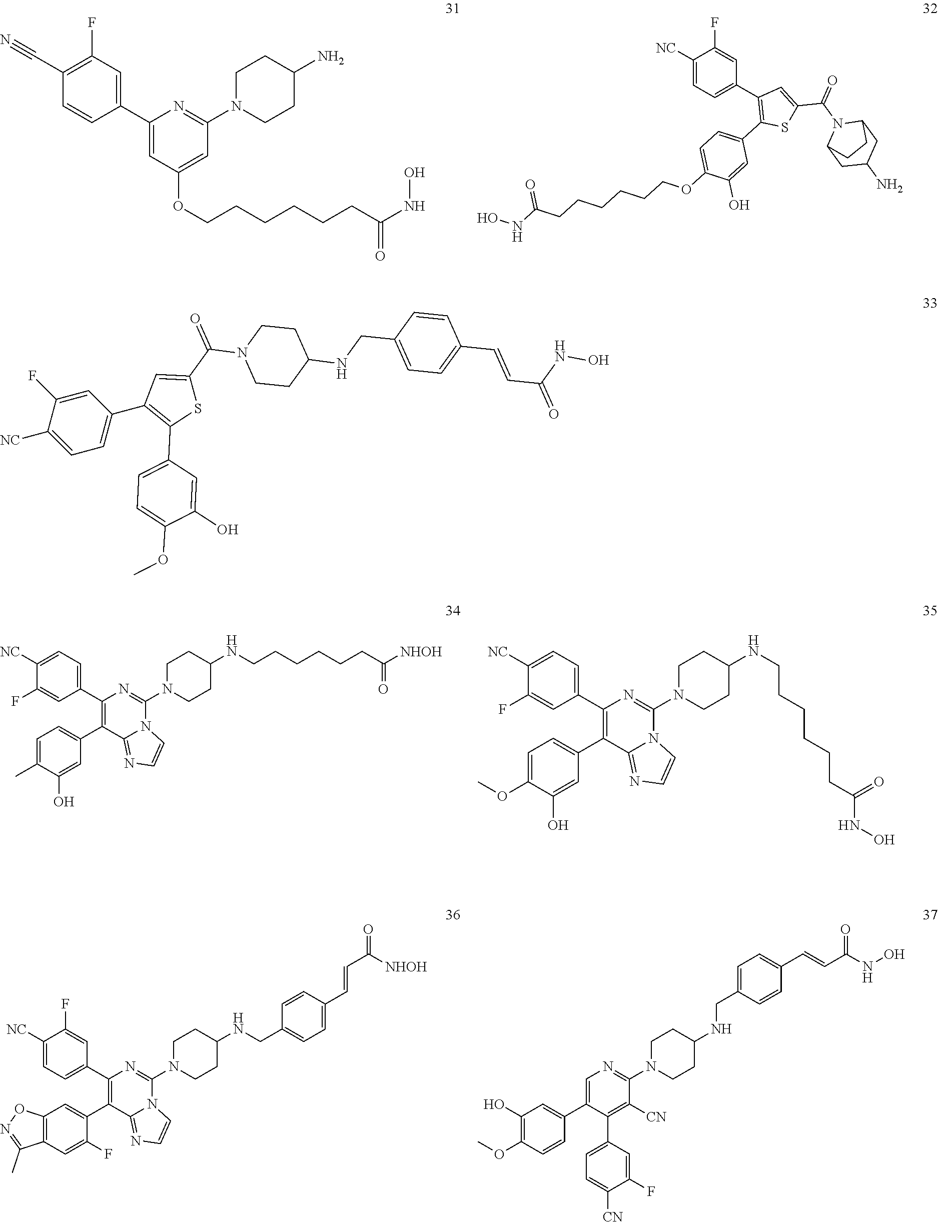

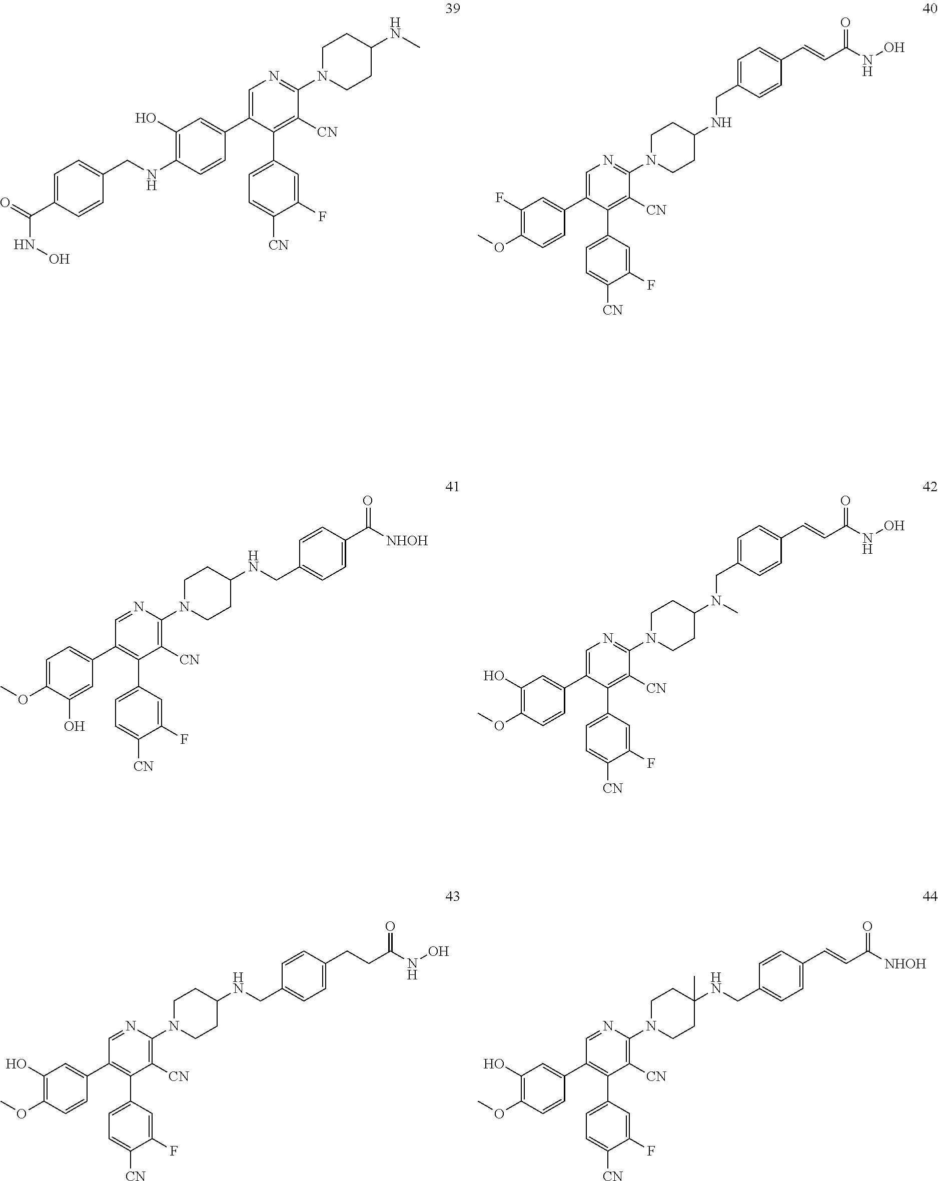

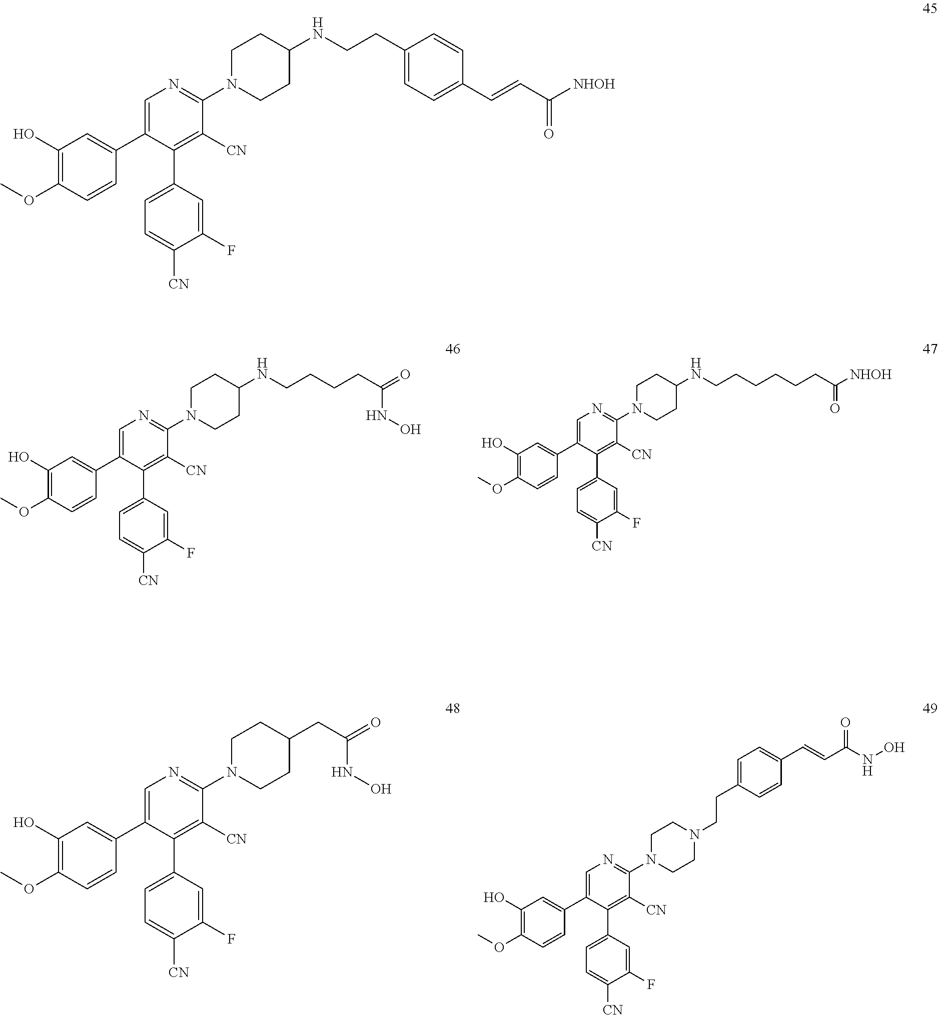

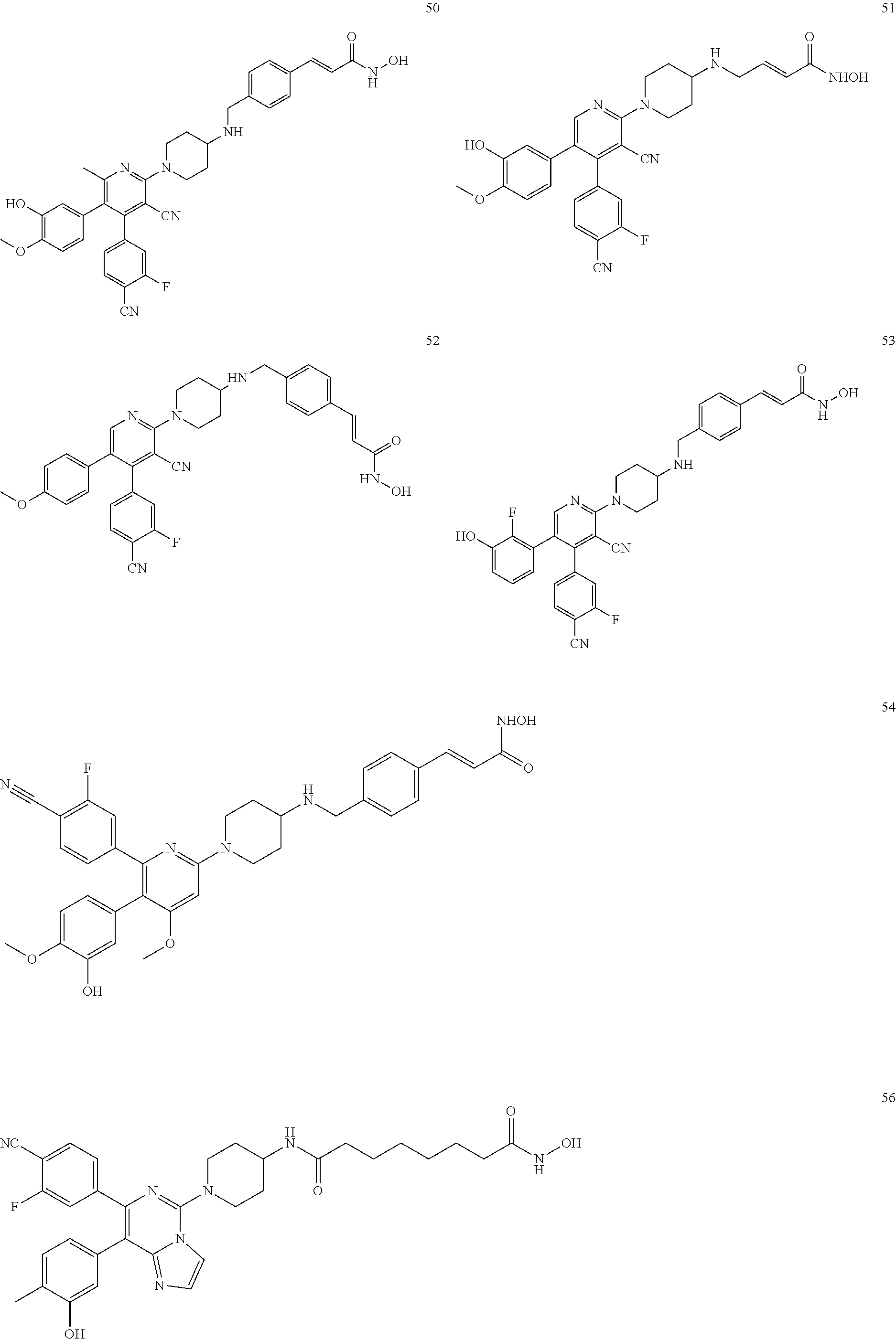

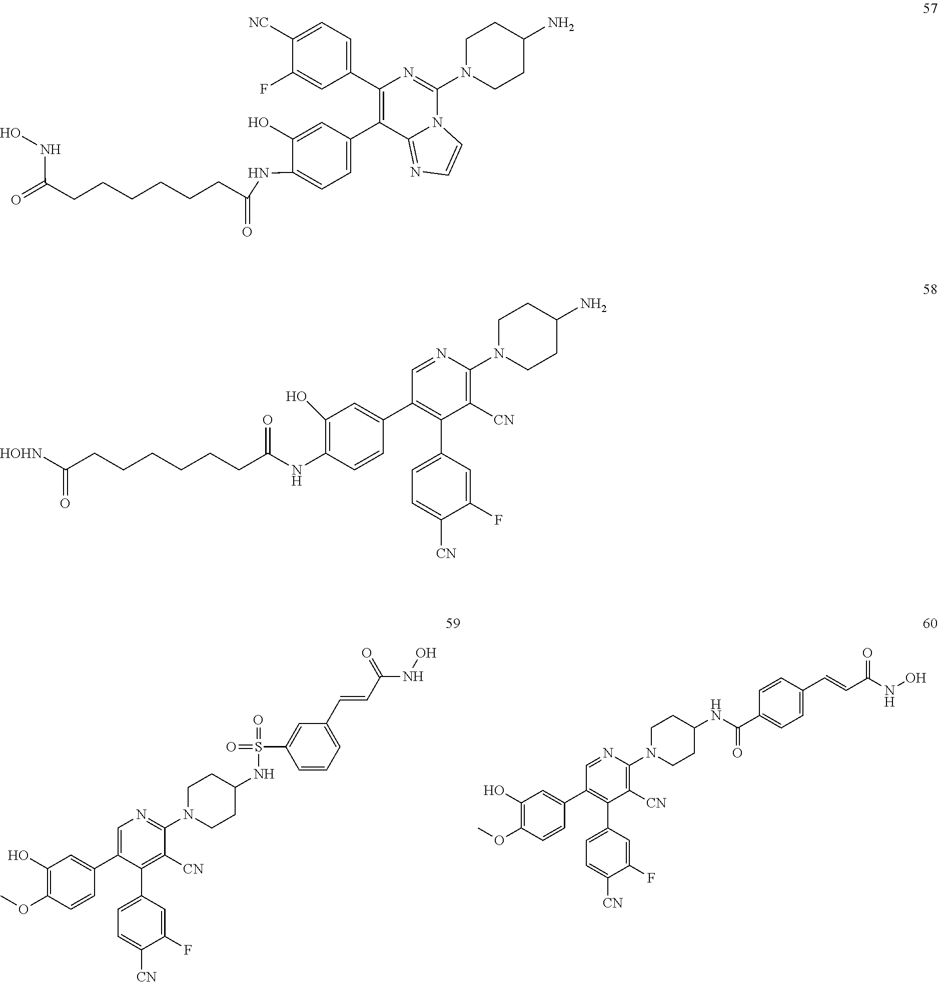

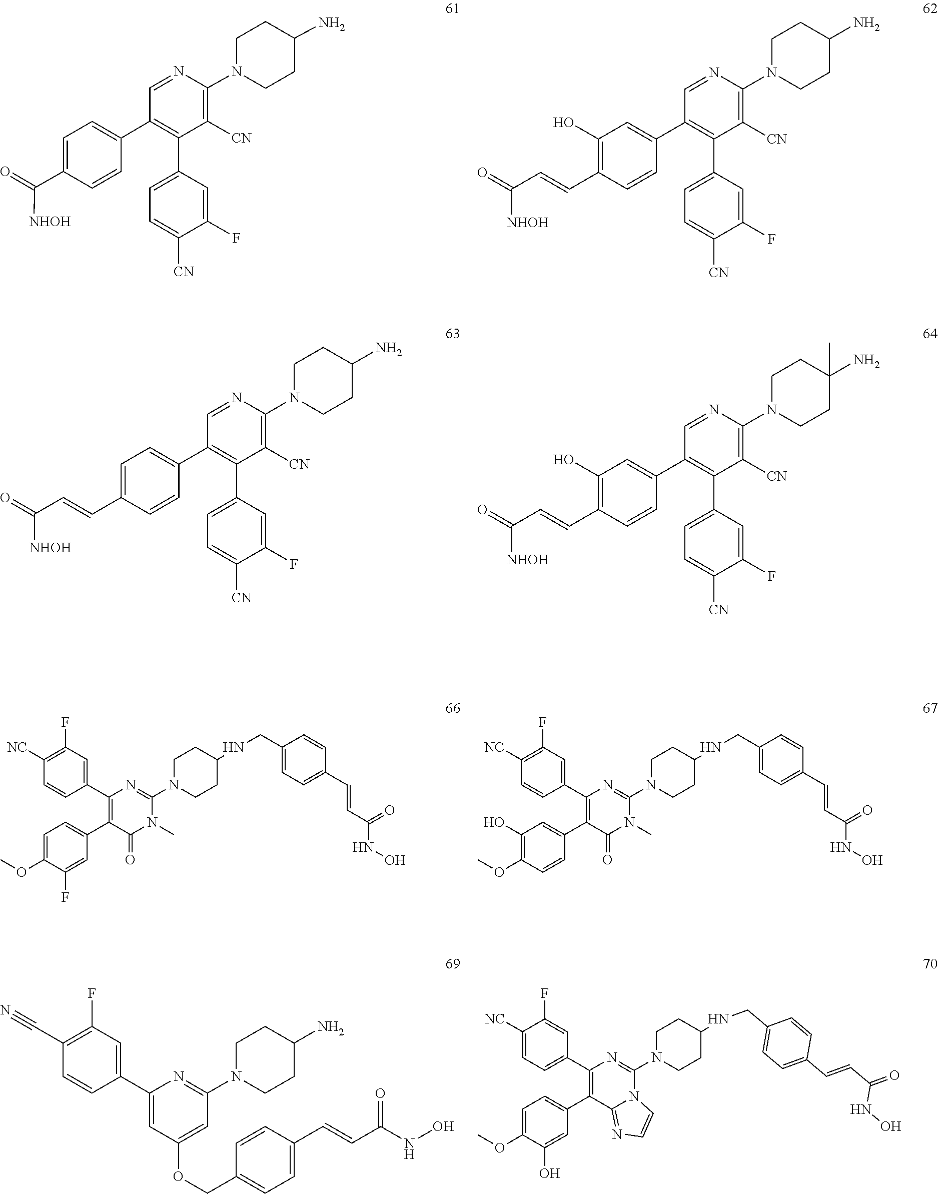

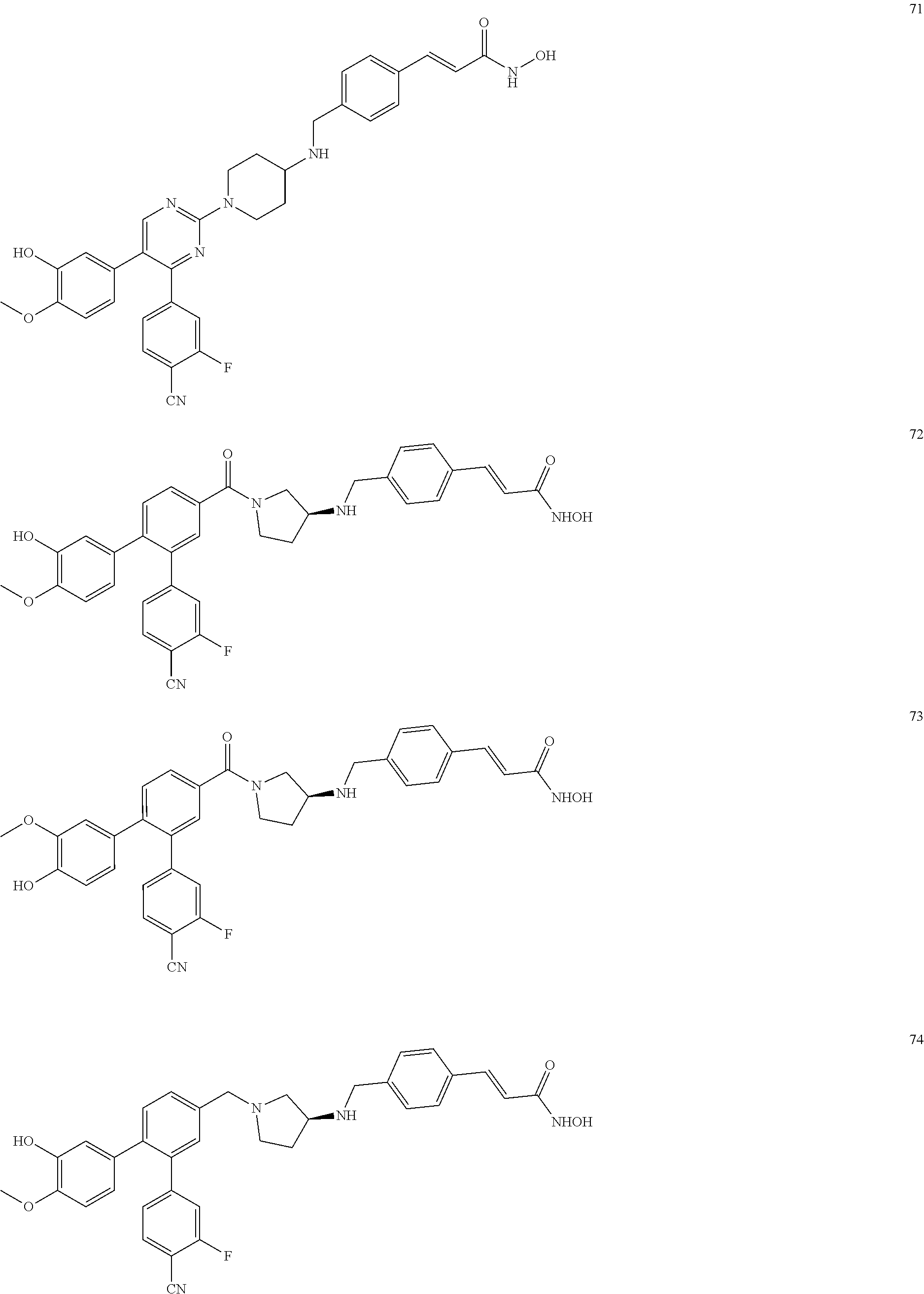

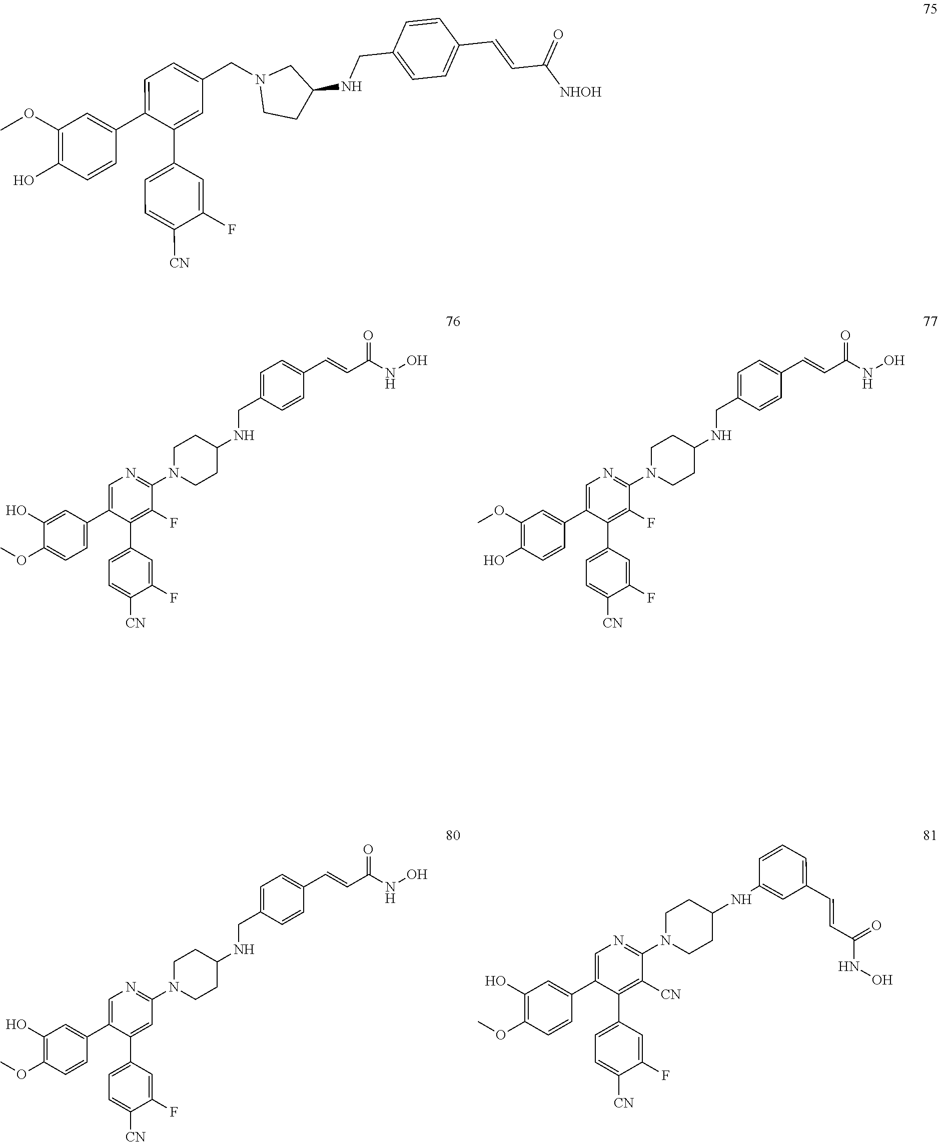

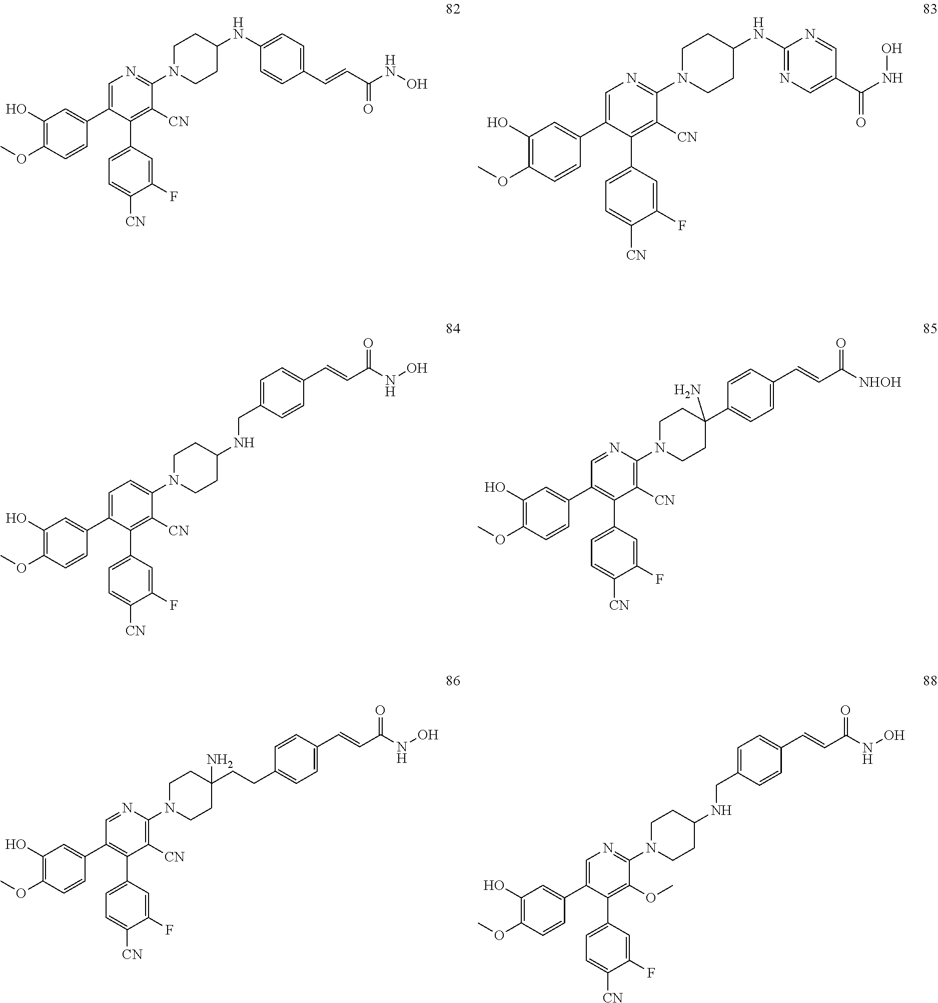

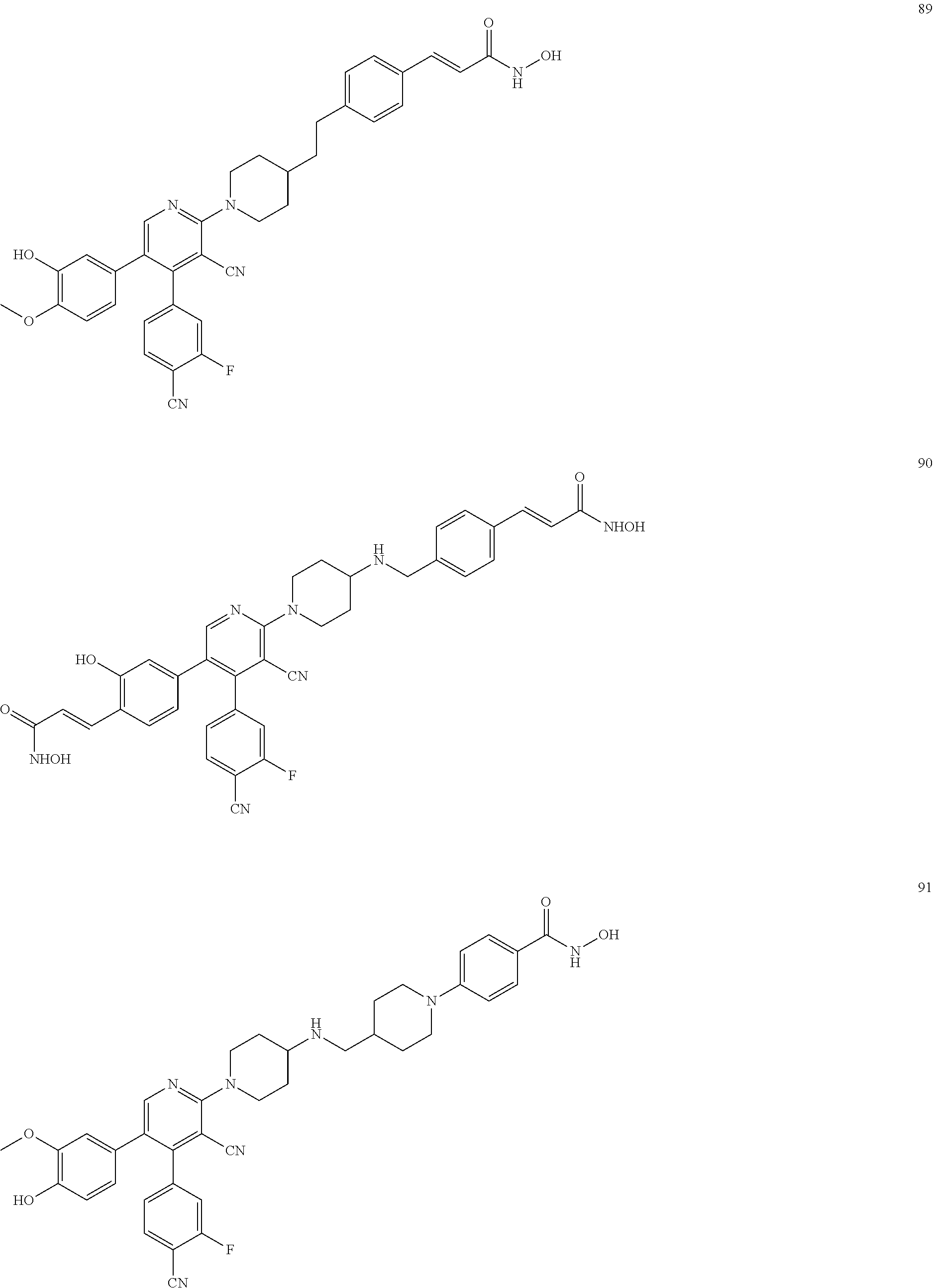

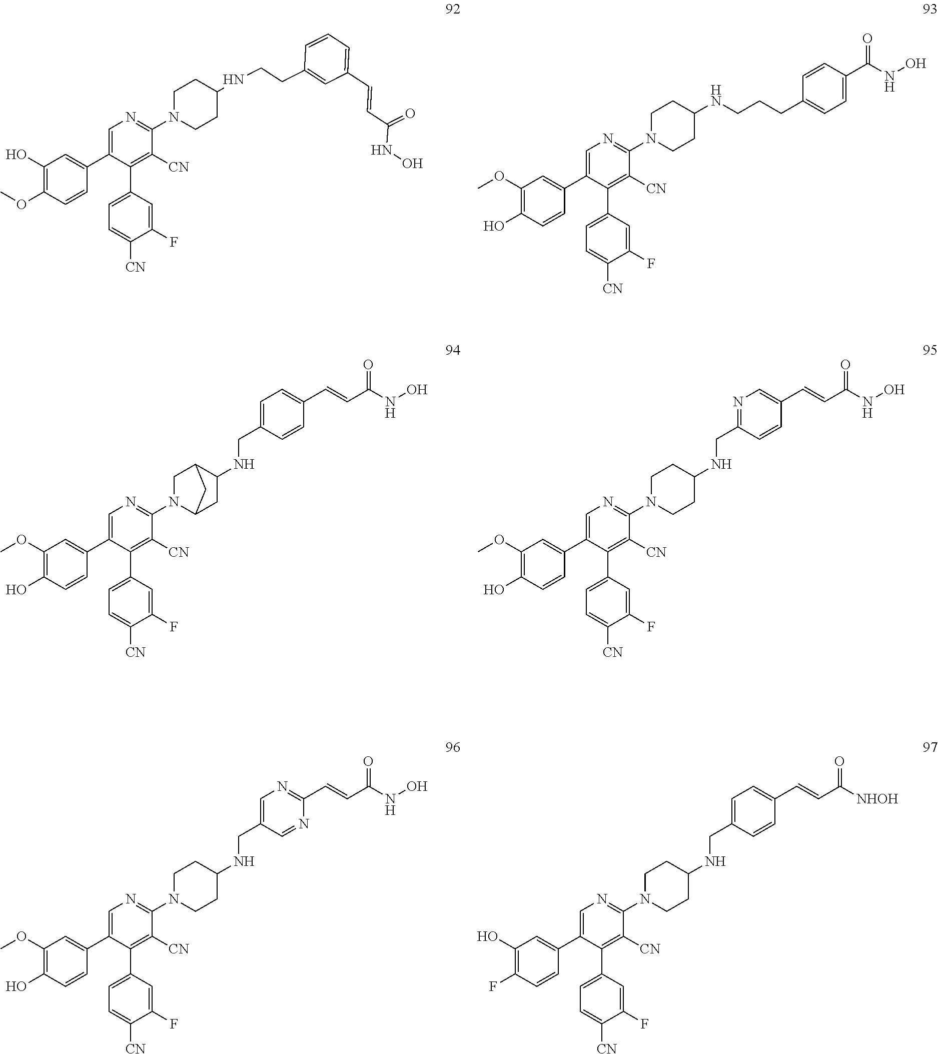

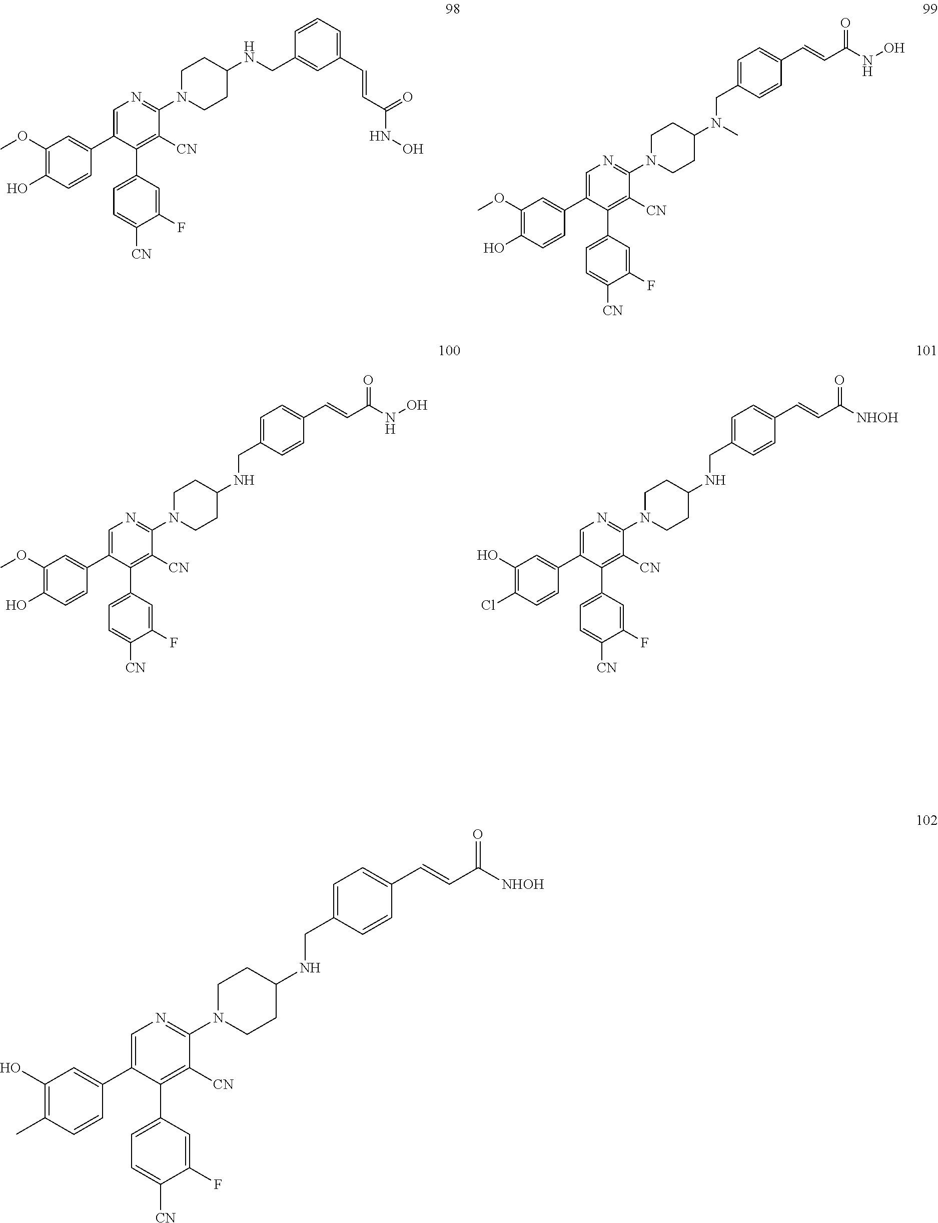

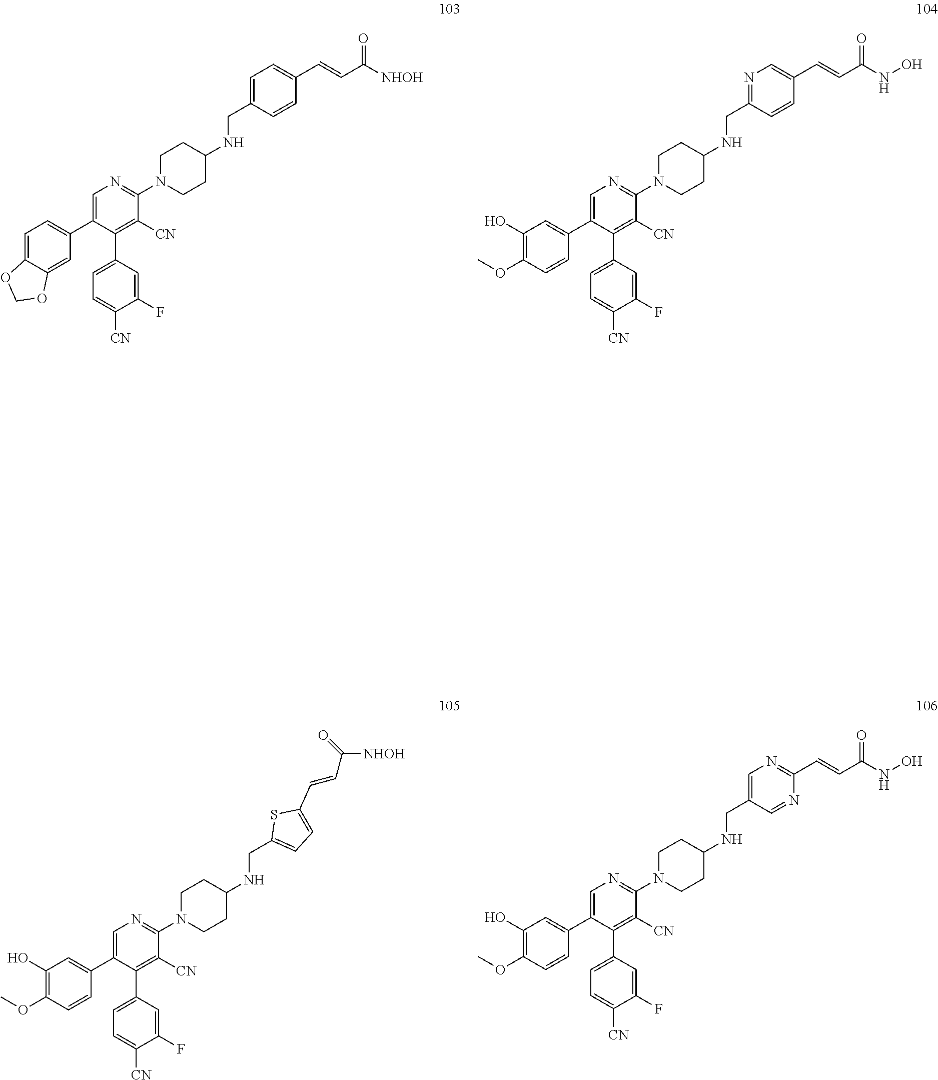

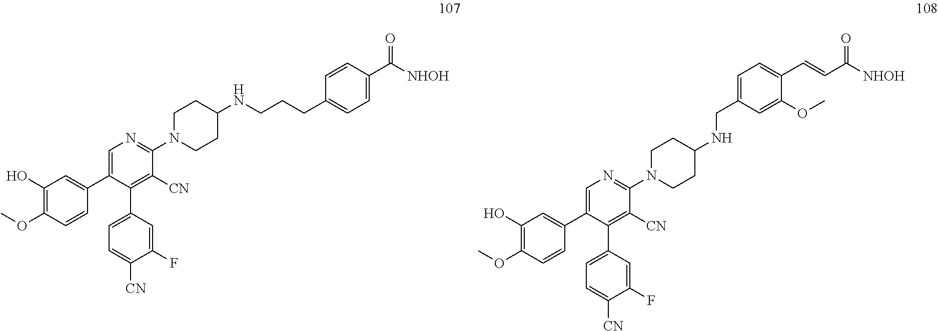

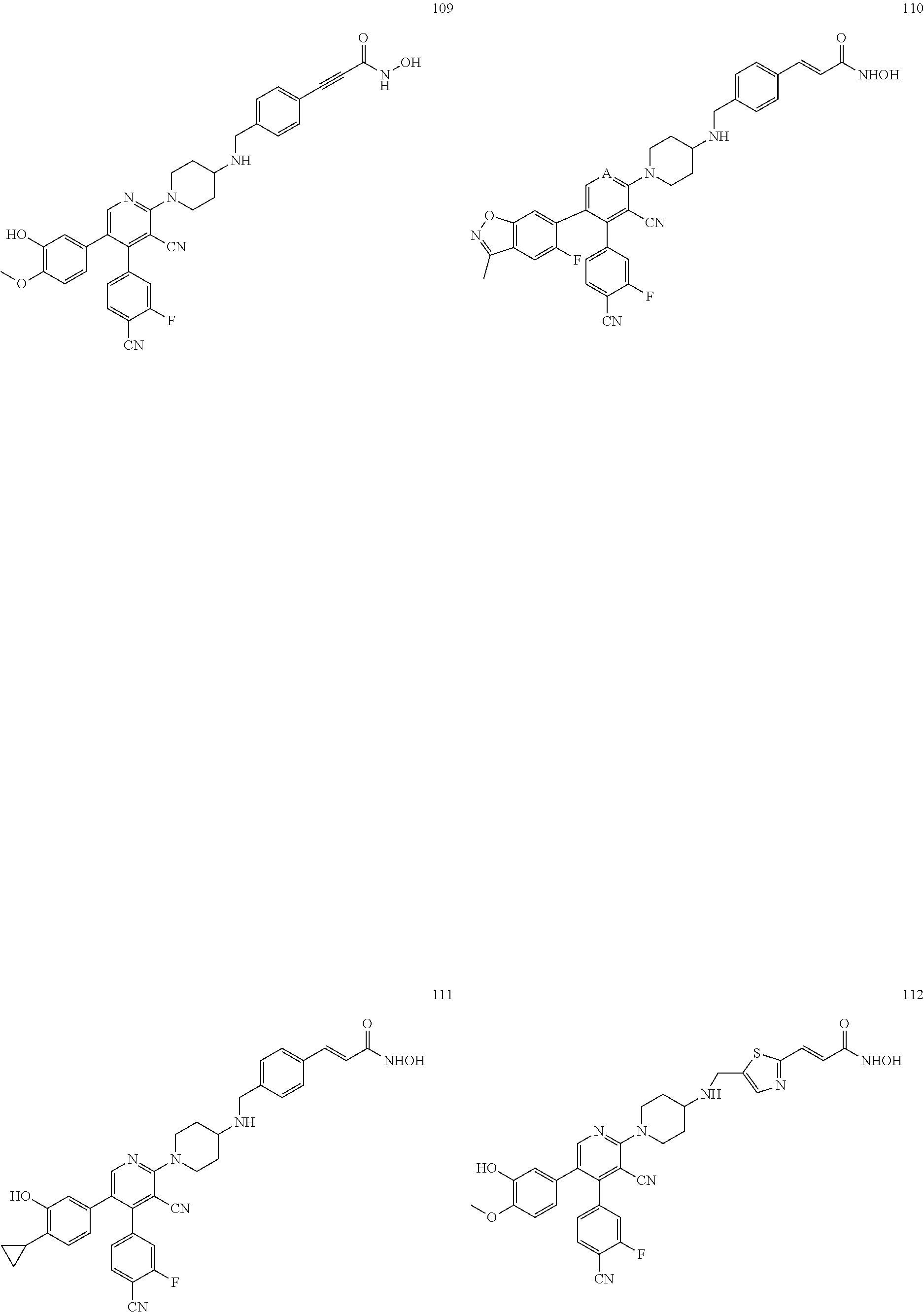

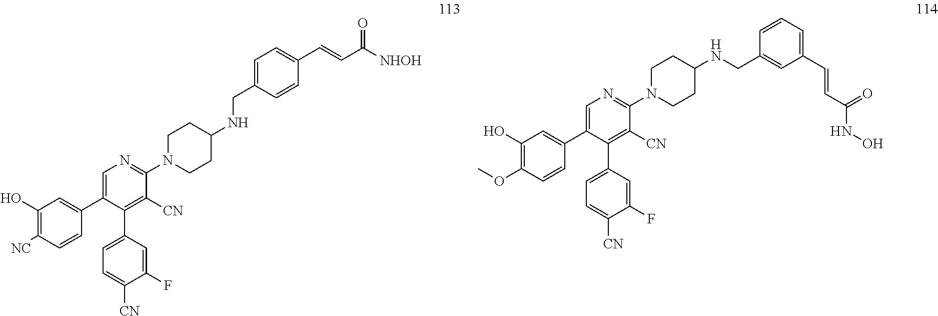

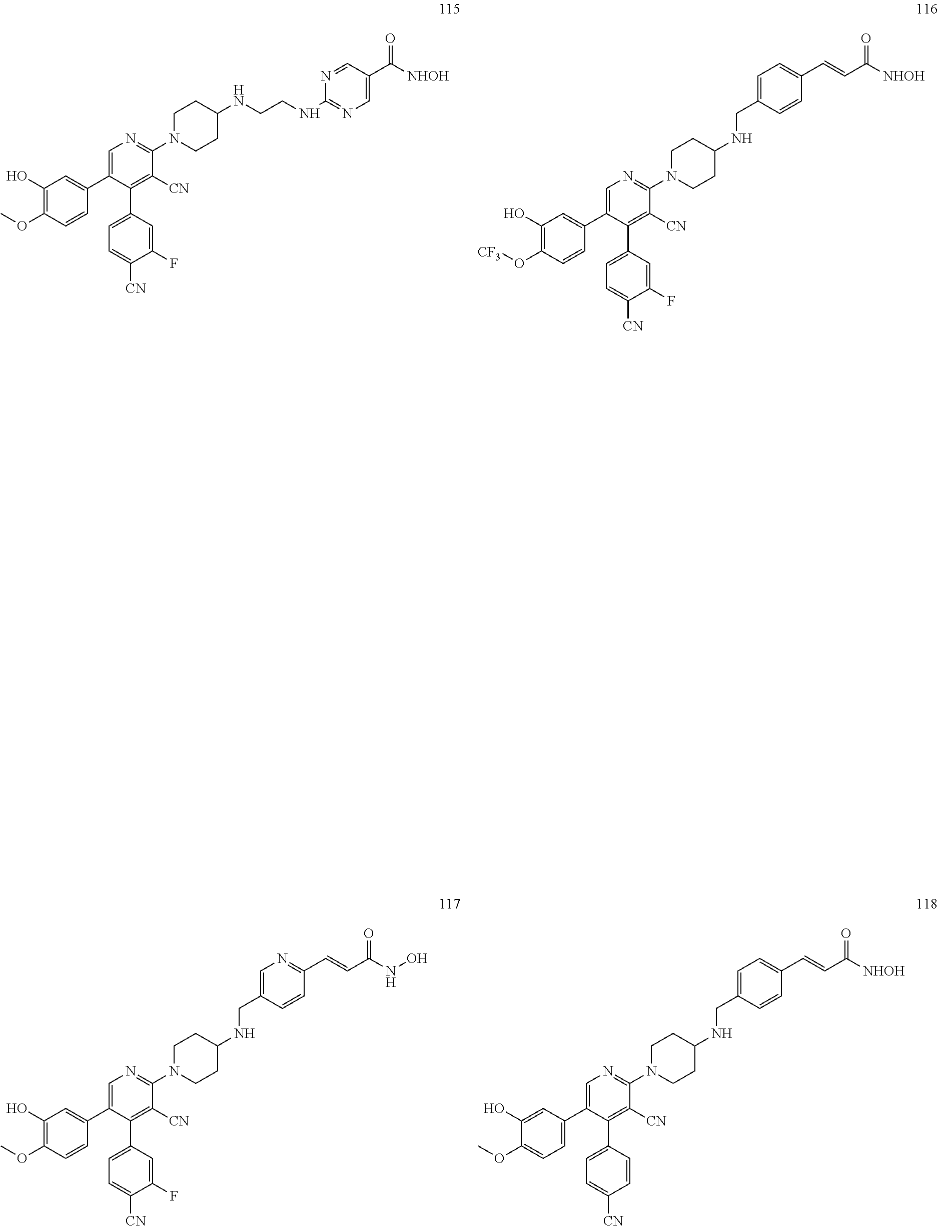

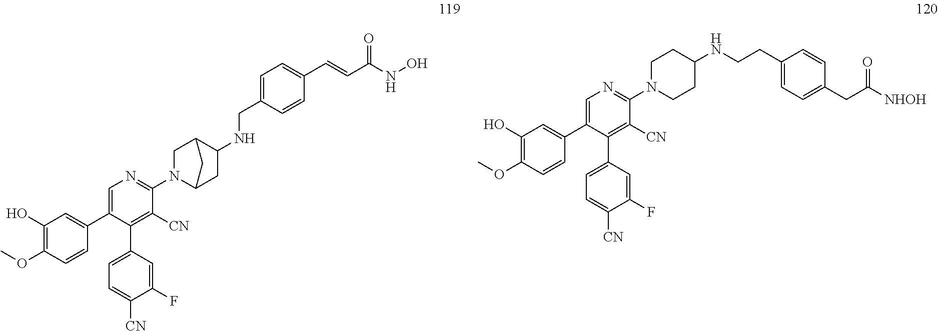

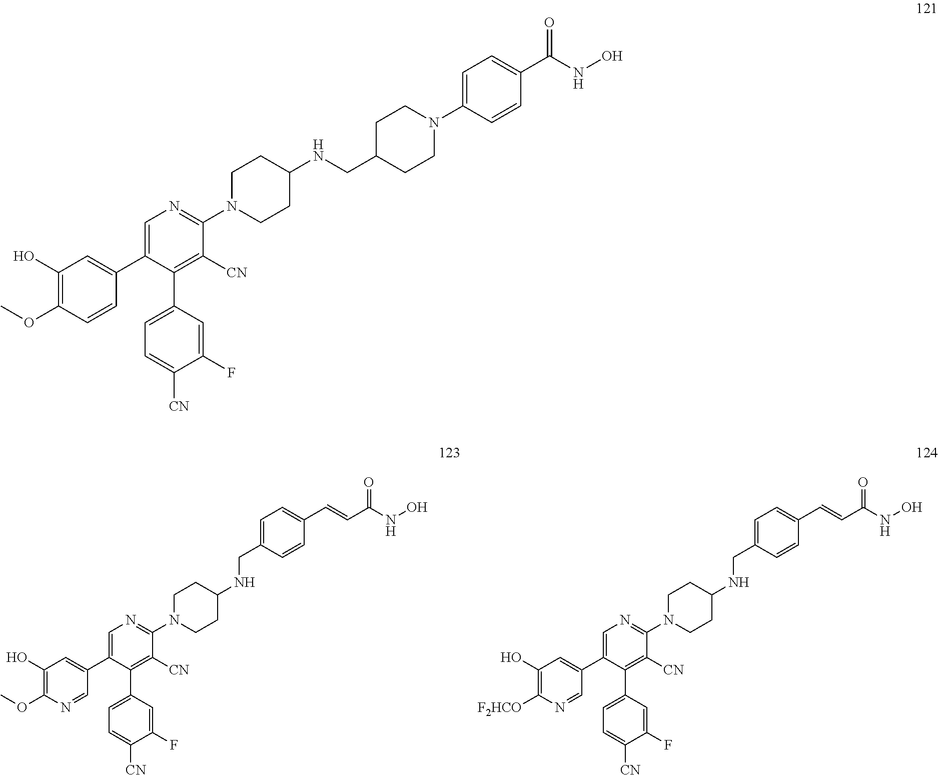 [SMILES: COc1ccc(-c2cnc(N3CCC(NCC4CCN(c5ccc(C(=O)NO)cc5)CC4)CC3)c(C#N)c2-c2ccc(C#N)c(F)c2)cc1O ; COc1ncc(-c2cnc(N3CCC(NCc4ccc(/C=C/C(=O)NO)cc4)CC3)c(C#N)c2-c2ccc(C#N)c(F)c2)cc1O ; N#Cc1ccc(-c2c(-c3cnc(OC(F)F)c(O)c3)cnc(N3CCC(NCc4ccc(/C=C/C(=O)NO)cc4)CC3)c2C#N)cc1F]

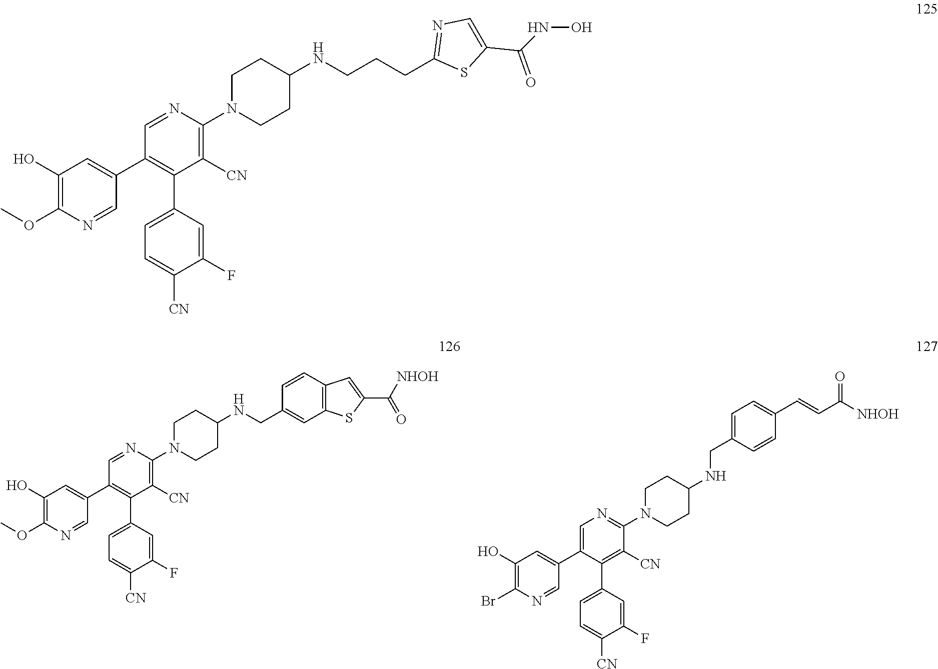

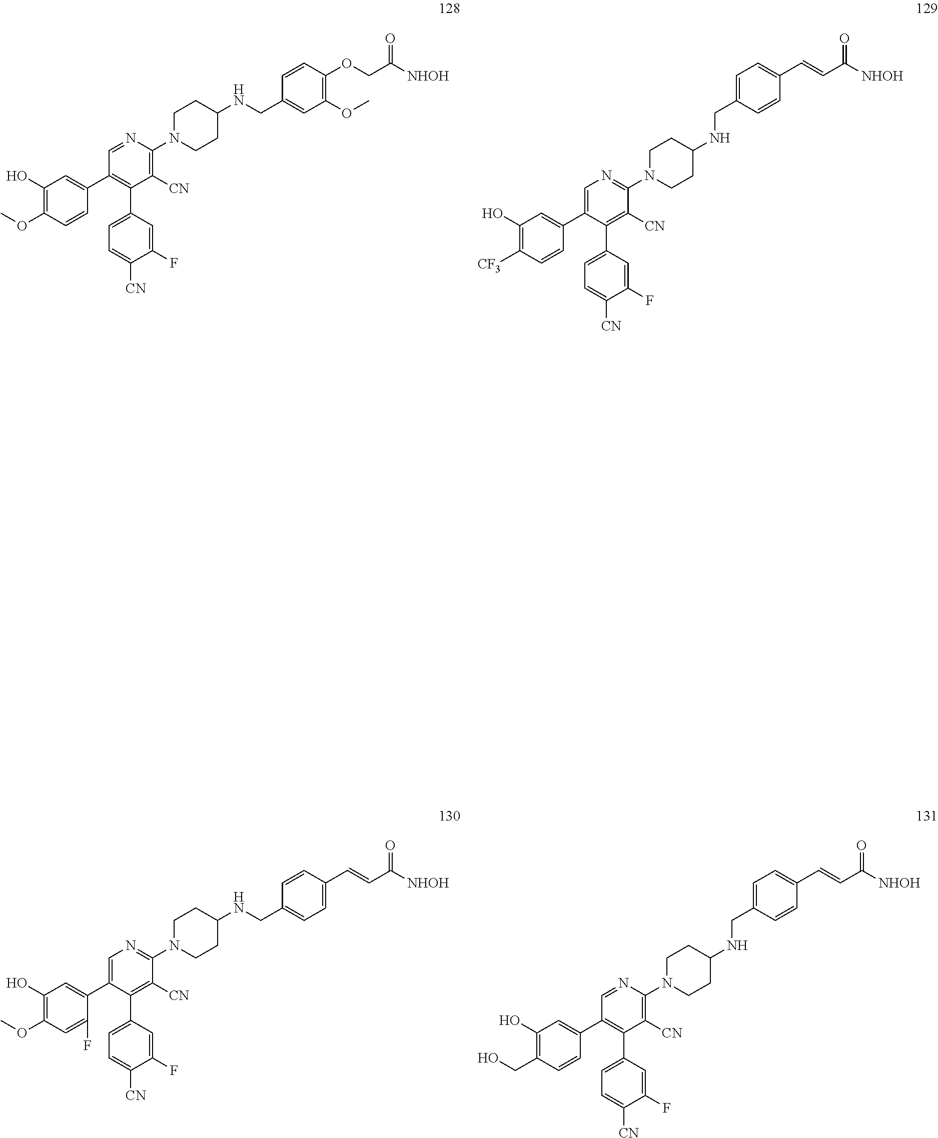

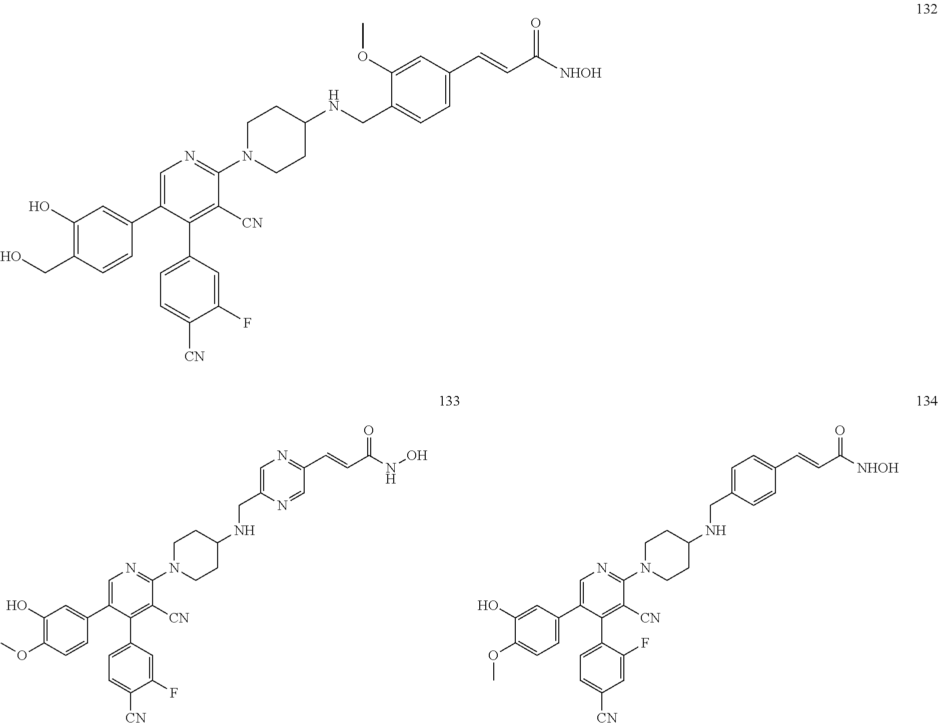

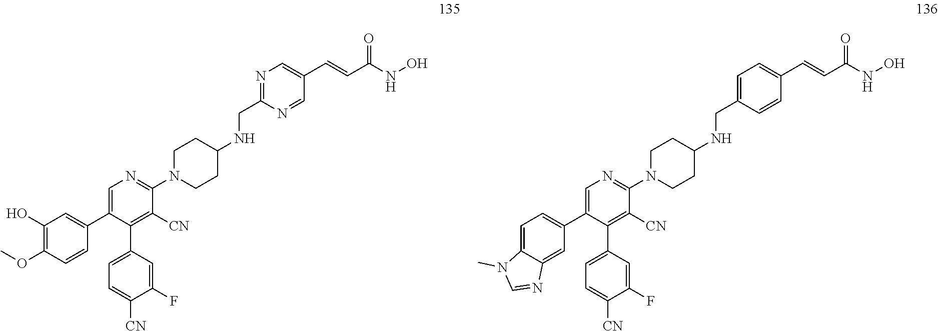 [SMILES: COc1ccc(-c2cnc(N3CCC(NCc4ncc(/C=C/C(=O)NO)cn4)CC3)c(C#N)c2-c2ccc(C#N)c(F)c2)cc1O ; Cn1cnc2cc(-c3cnc(N4CCC(NCc5ccc(/C=C/C(=O)NO)cc5)CC4)c(C#N)c3-c3ccc(C#N)c(F)c3)ccc21]

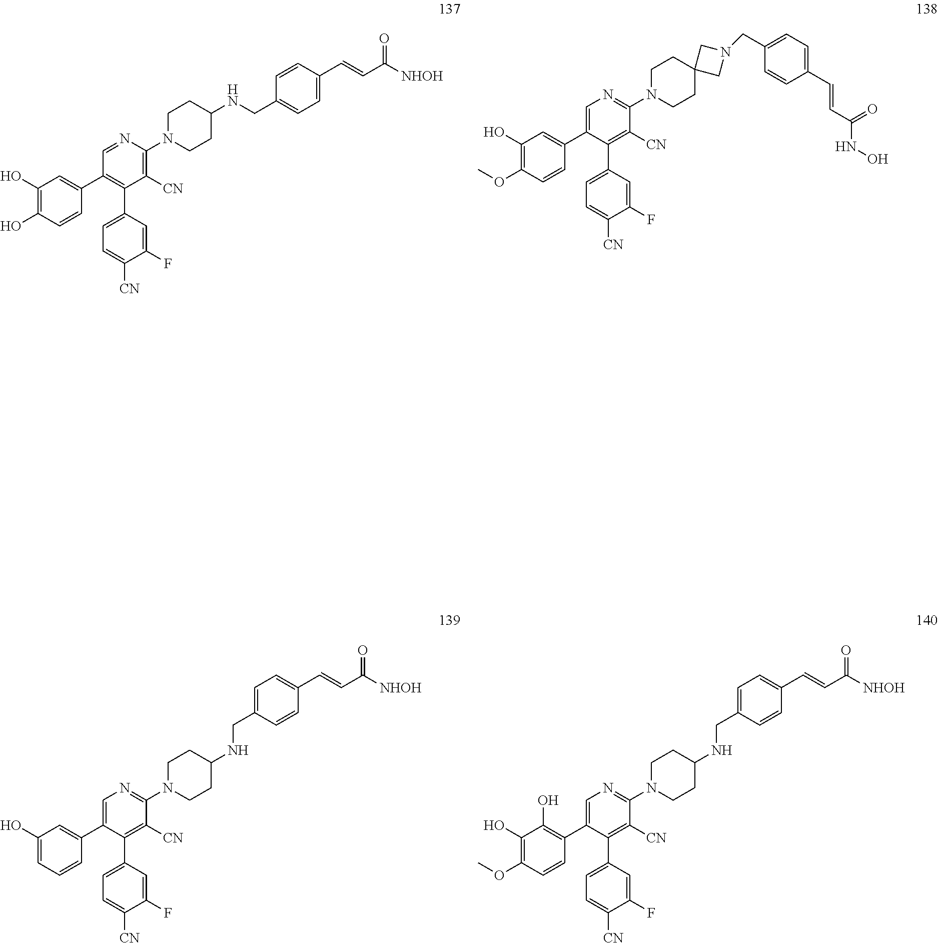

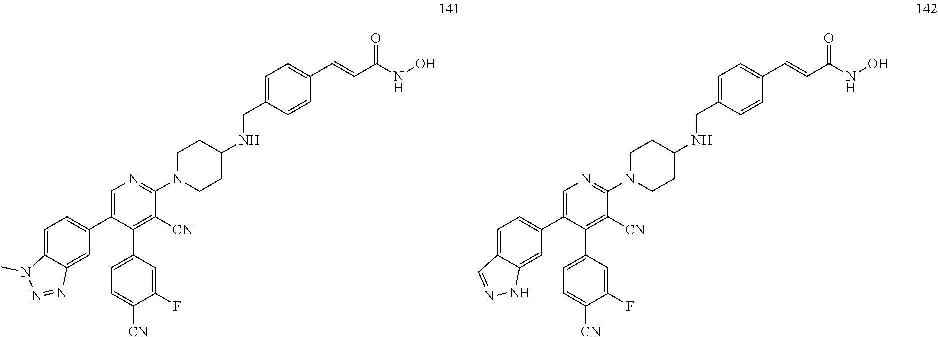 [SMILES: Cn1nnc2cc(-c3cnc(N4CCC(NCc5ccc(/C=C/C(=O)NO)cc5)CC4)c(C#N)c3-c3ccc(C#N)c(F)c3)ccc21 ; N#Cc1ccc(-c2c(-c3ccc4cn[nH]c4c3)cnc(N3CCC(NCc4ccc(/C=C/C(=O)NO)cc4)CC3)c2C#N)cc1F]

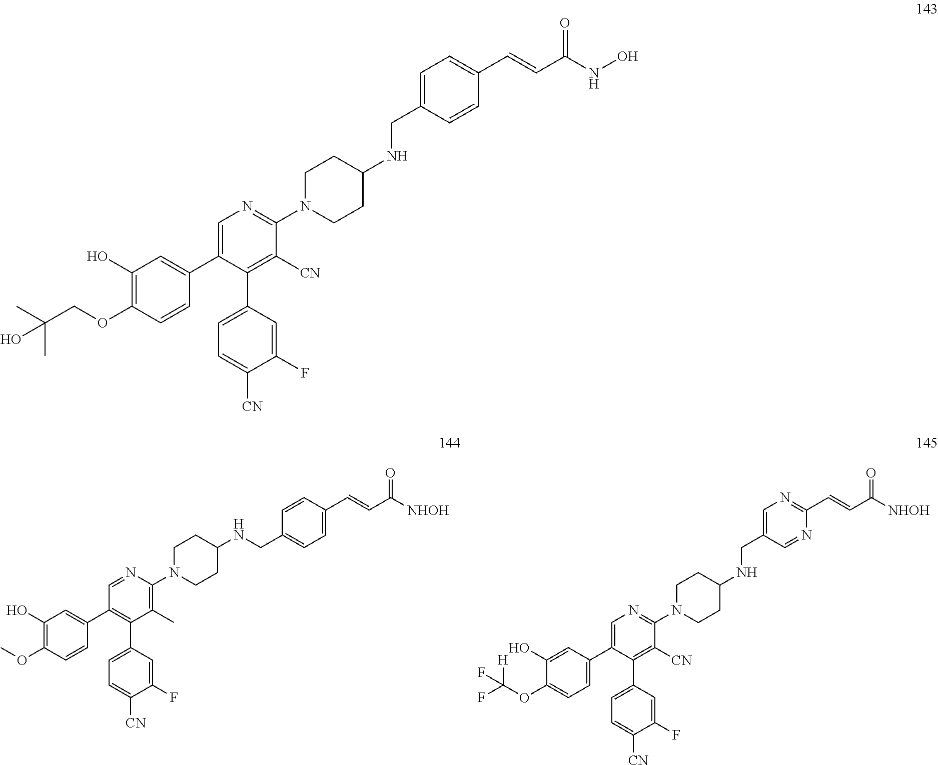

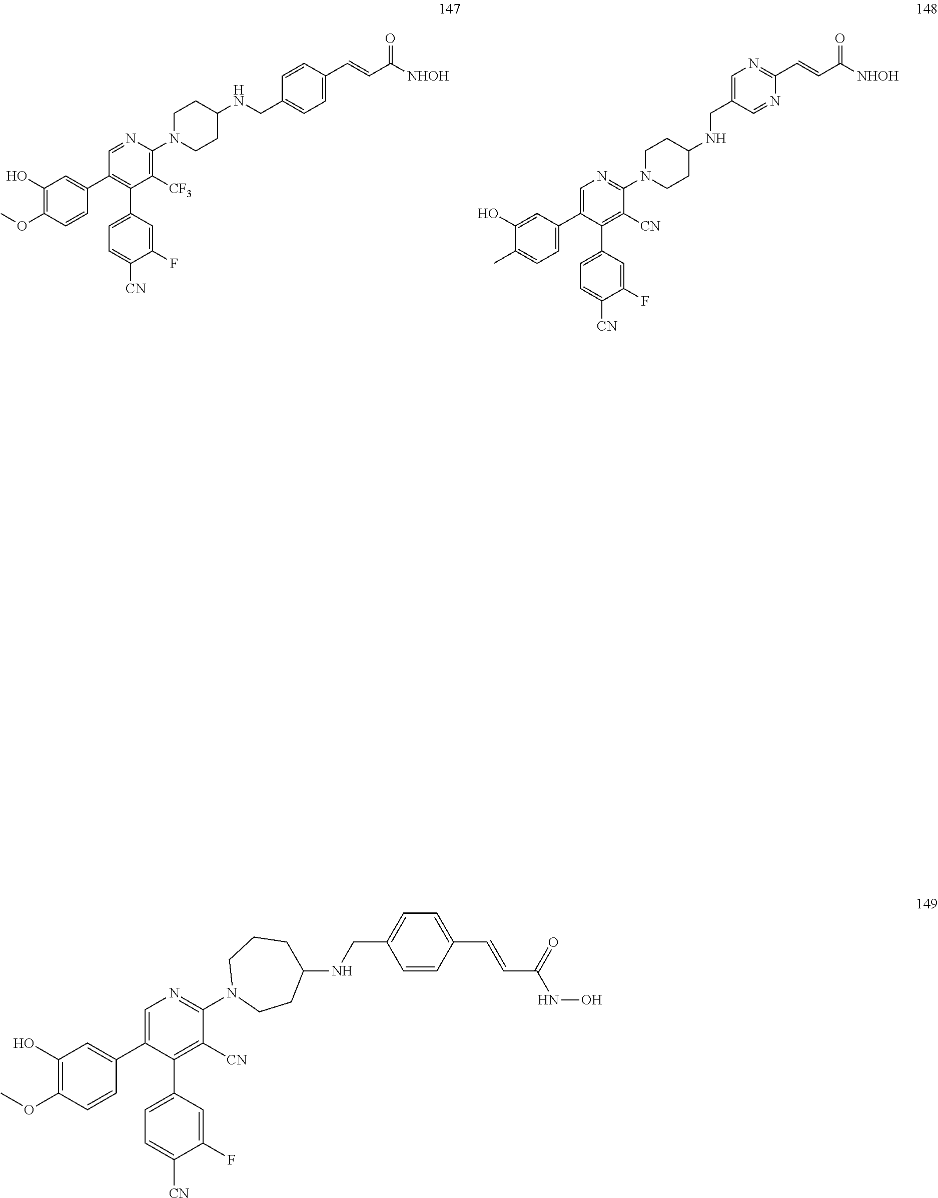

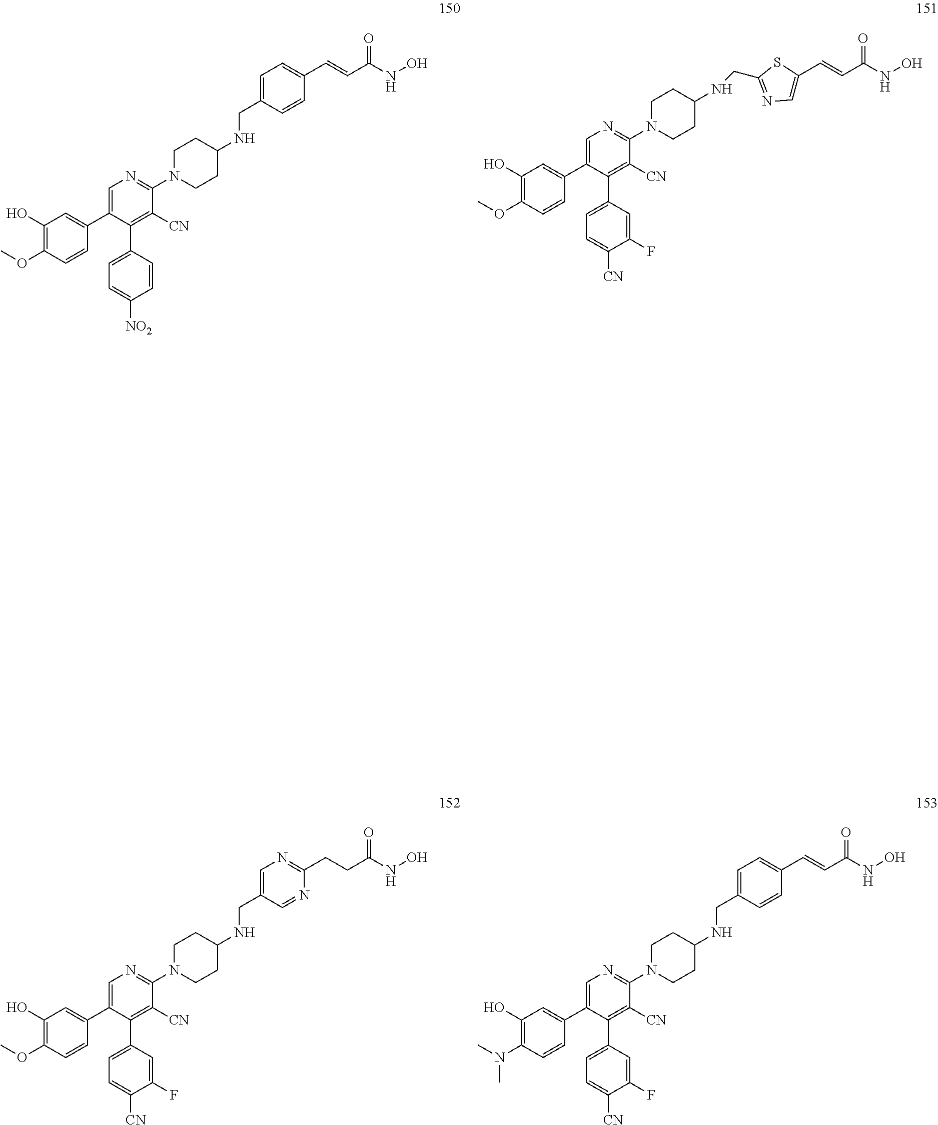 [SMILES: COc1ccc(-c2cnc(N3CCC(NCc4ccc(/C=C/C(=O)NO)cc4)CC3)c(C#N)c2-c2ccc([N+](=O)[O-])cc2)cc1O ; COc1ccc(-c2cnc(N3CCC(NCc4ncc(/C=C/C(=O)NO)s4)CC3)c(C#N)c2-c2ccc(C#N)c(F)c2)cc1O ; COc1ccc(-c2cnc(N3CCC(NCc4cnc(CCC(=O)NO)nc4)CC3)c(C#N)c2-c2ccc(C#N)c(F)c2)cc1O ; CN(C)c1ccc(-c2cnc(N3CCC(NCc4ccc(/C=C/C(=O)NO)cc4)CC3)c(C#N)c2-c2ccc(C#N)c(F)c2)cc1O]

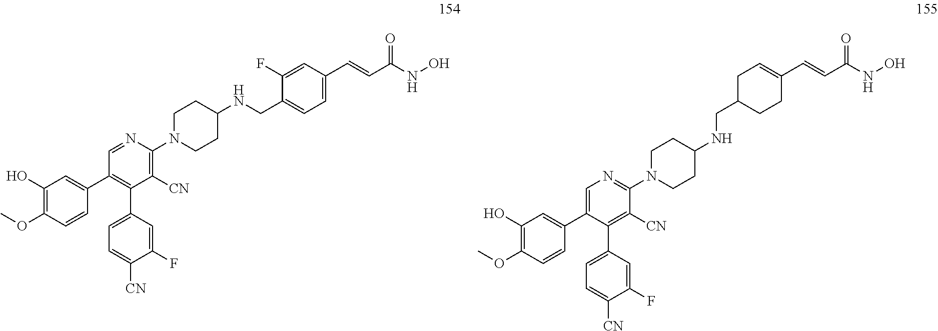 [SMILES: COc1ccc(-c2cnc(N3CCC(NCc4ccc(/C=C/C(=O)NO)cc4F)CC3)c(C#N)c2-c2ccc(C#N)c(F)c2)cc1O ; COc1ccc(-c2cnc(N3CCC(NCC4CC=C(/C=C/C(=O)NO)CC4)CC3)c(C#N)c2-c2ccc(C#N)c(F)c2)cc1O]

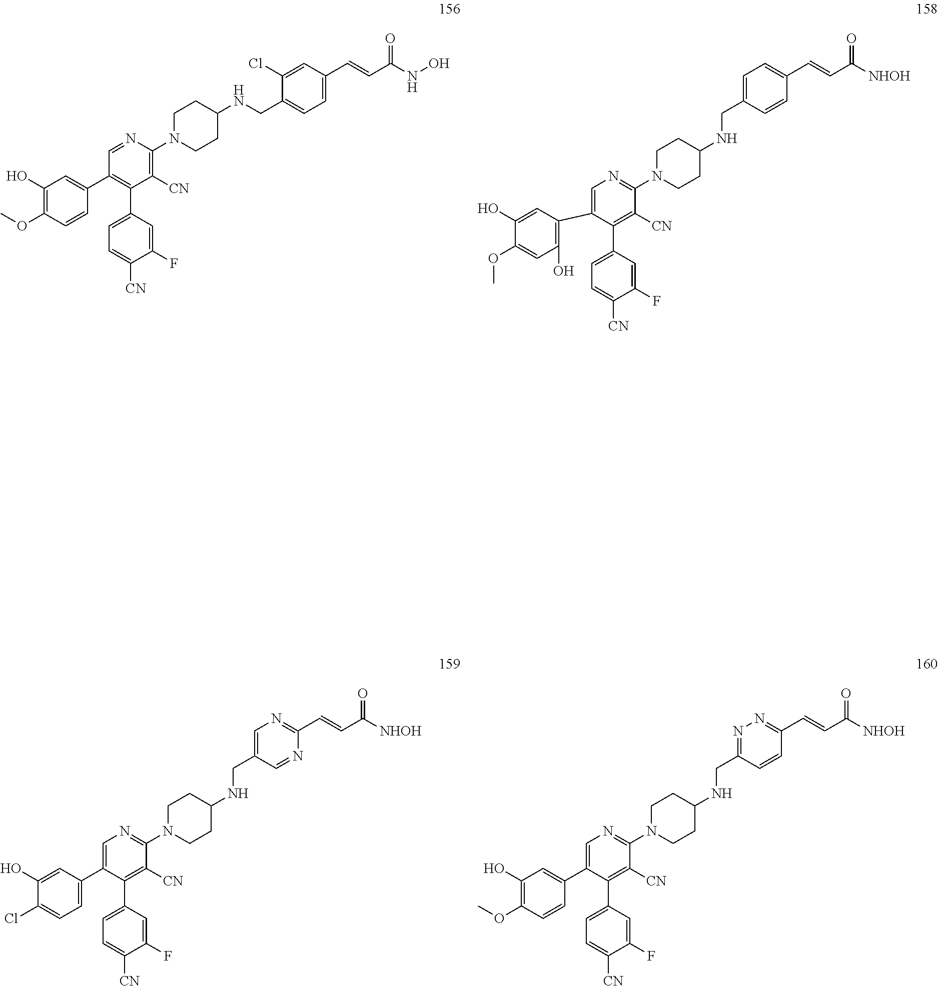

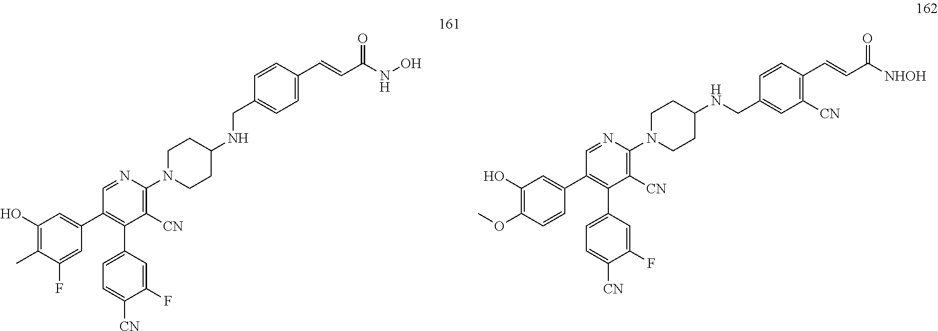

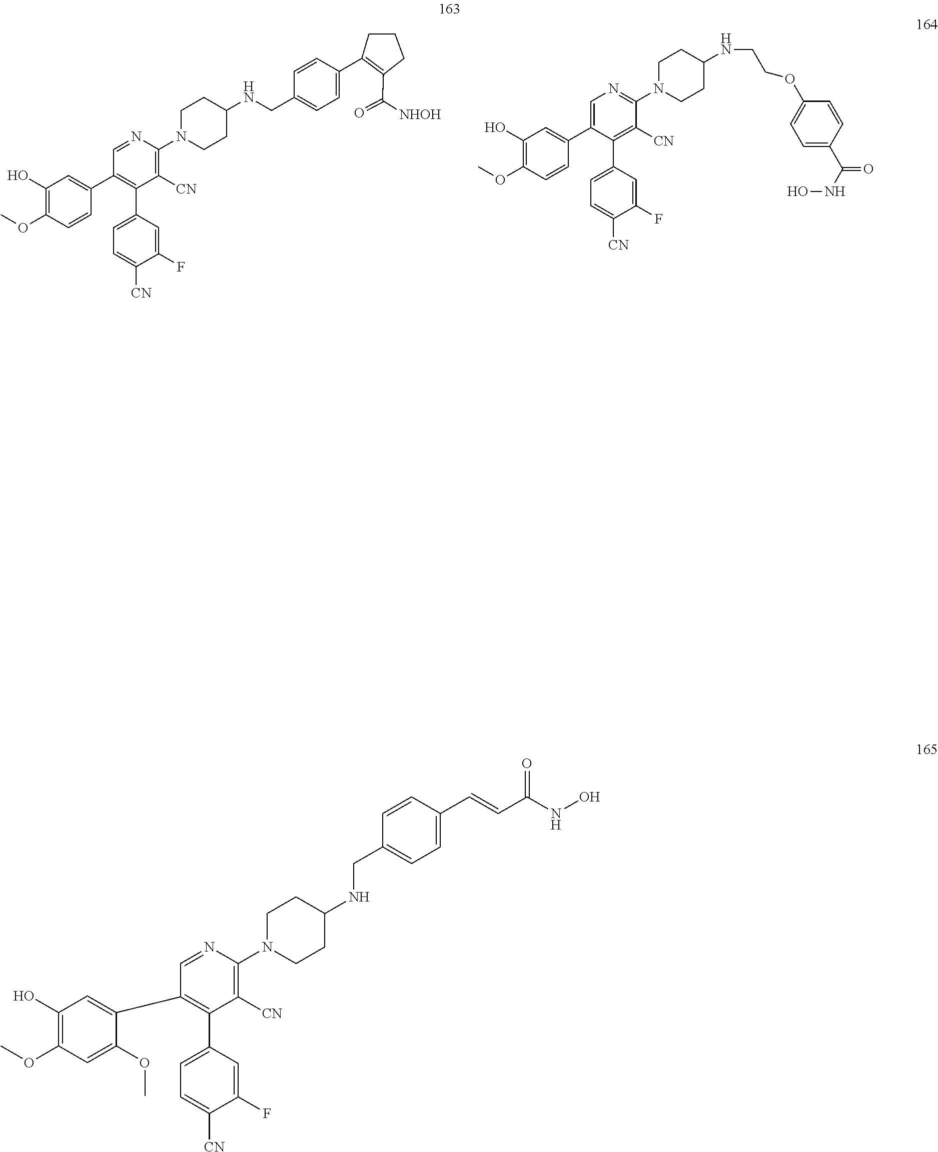

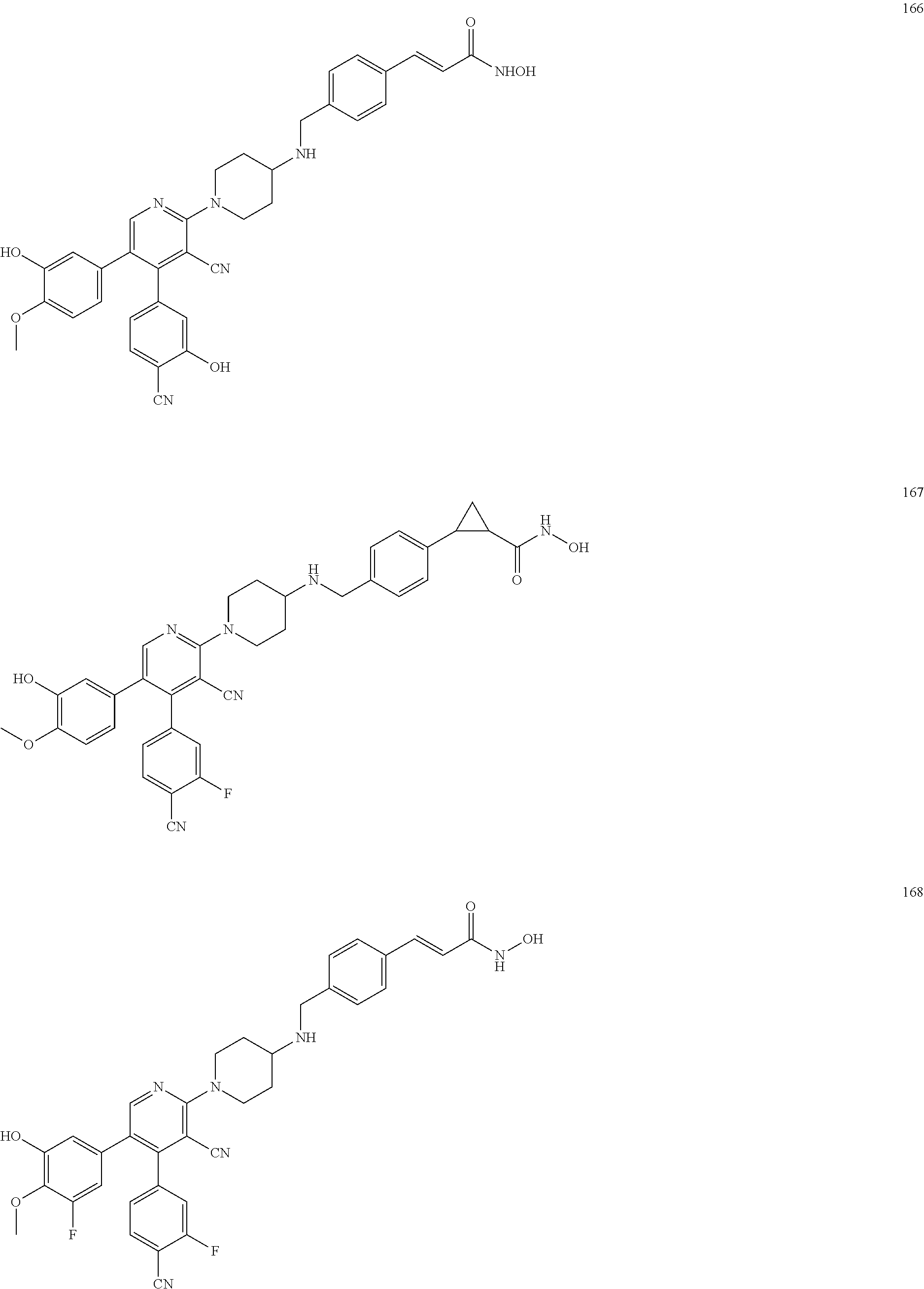

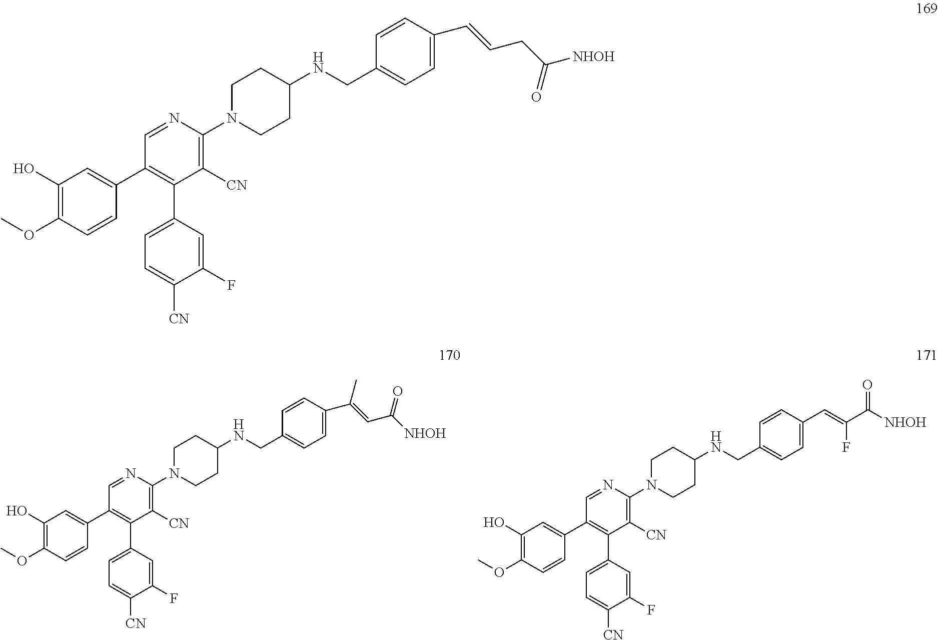

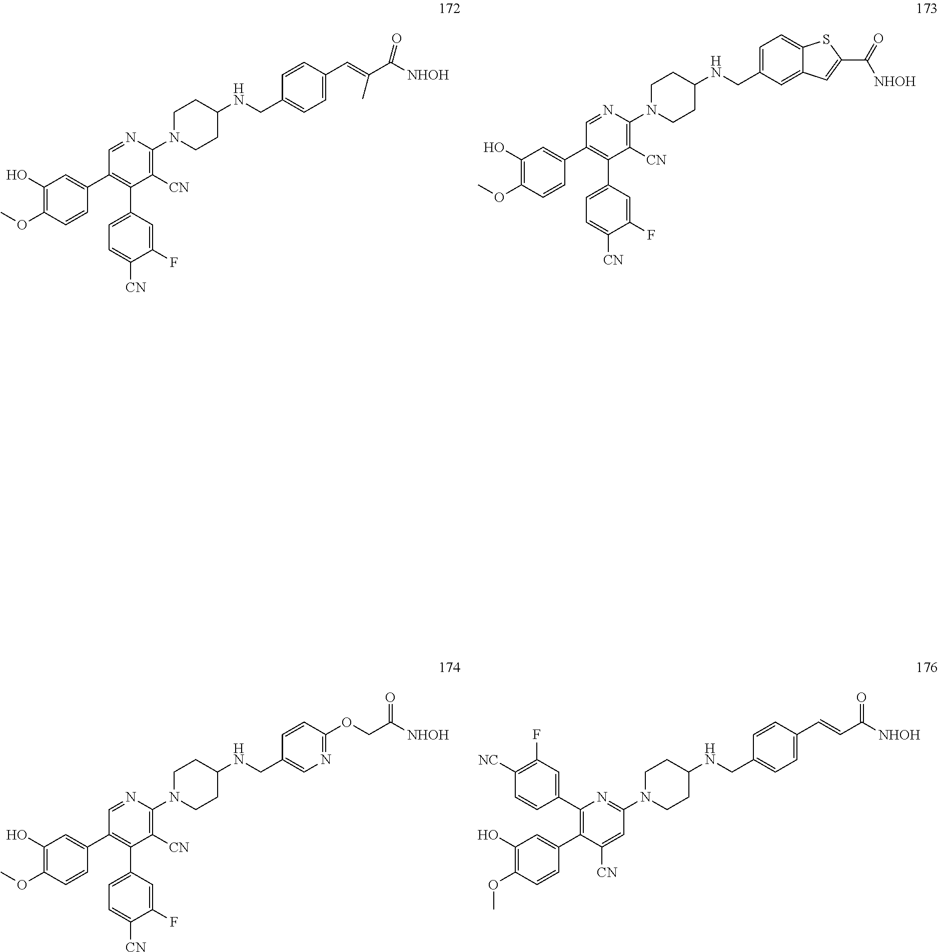

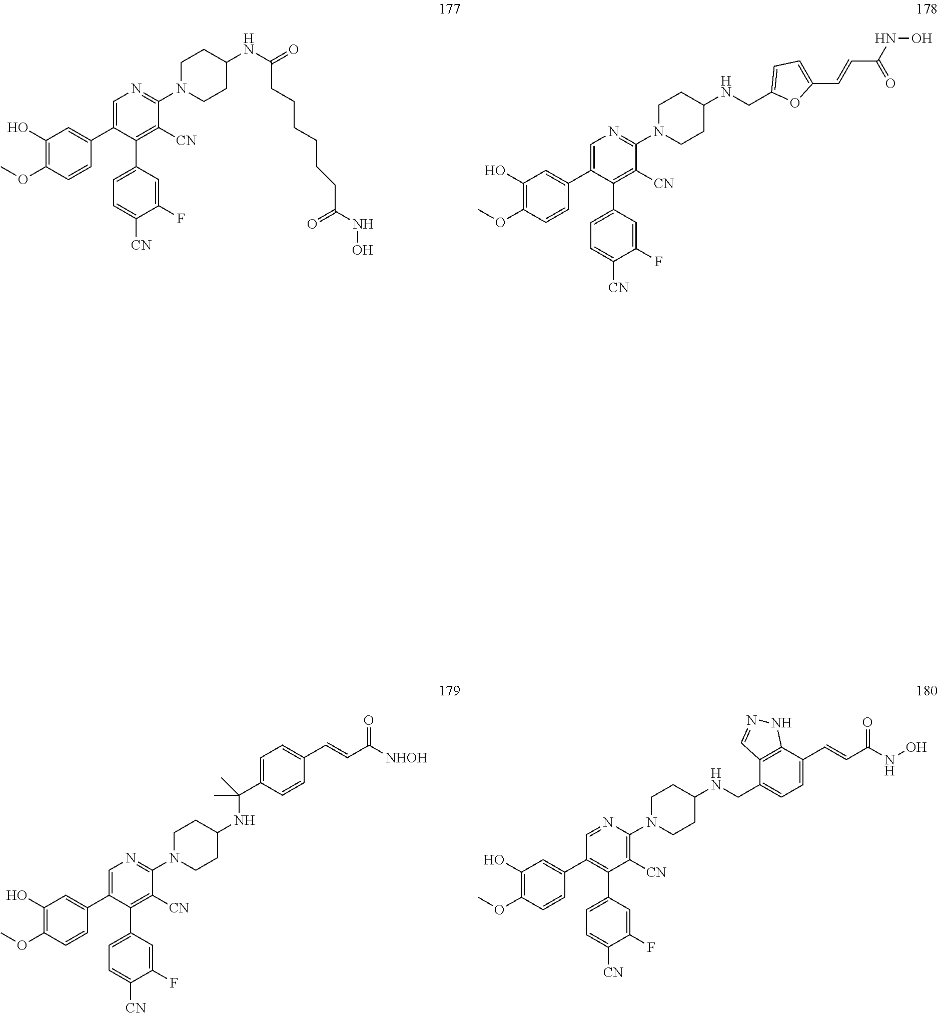

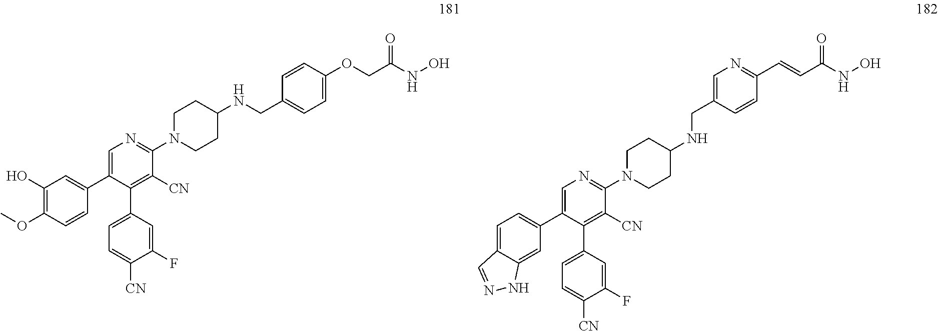 [SMILES: COc1ccc(-c2cnc(N3CCC(NCc4ccc(OCC(=O)NO)cc4)CC3)c(C#N)c2-c2ccc(C#N)c(F)c2)cc1O ; N#Cc1ccc(-c2c(-c3ccc4cn[nH]c4c3)cnc(N3CCC(NCc4ccc(/C=C/C(=O)NO)nc4)CC3)c2C#N)cc1F]

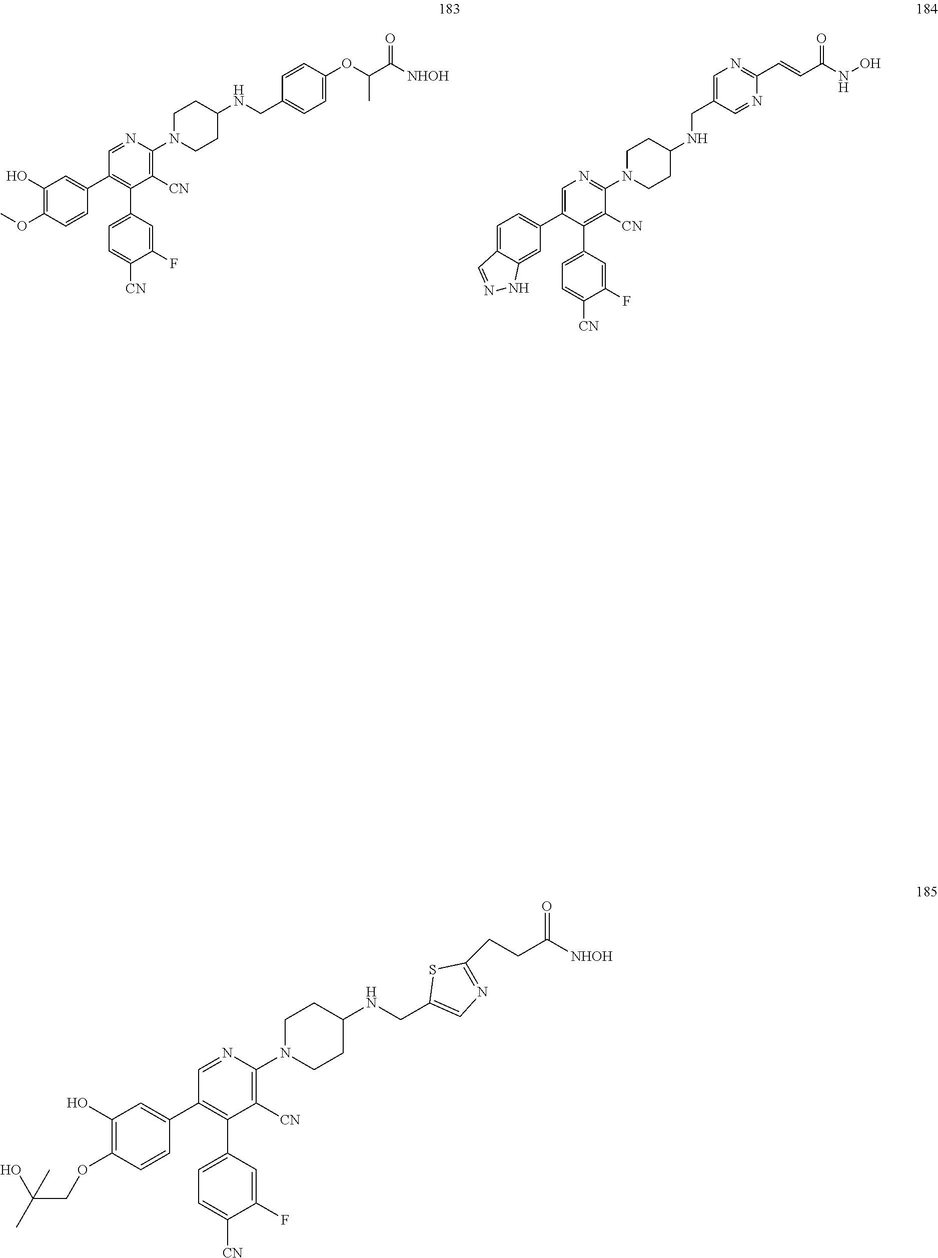

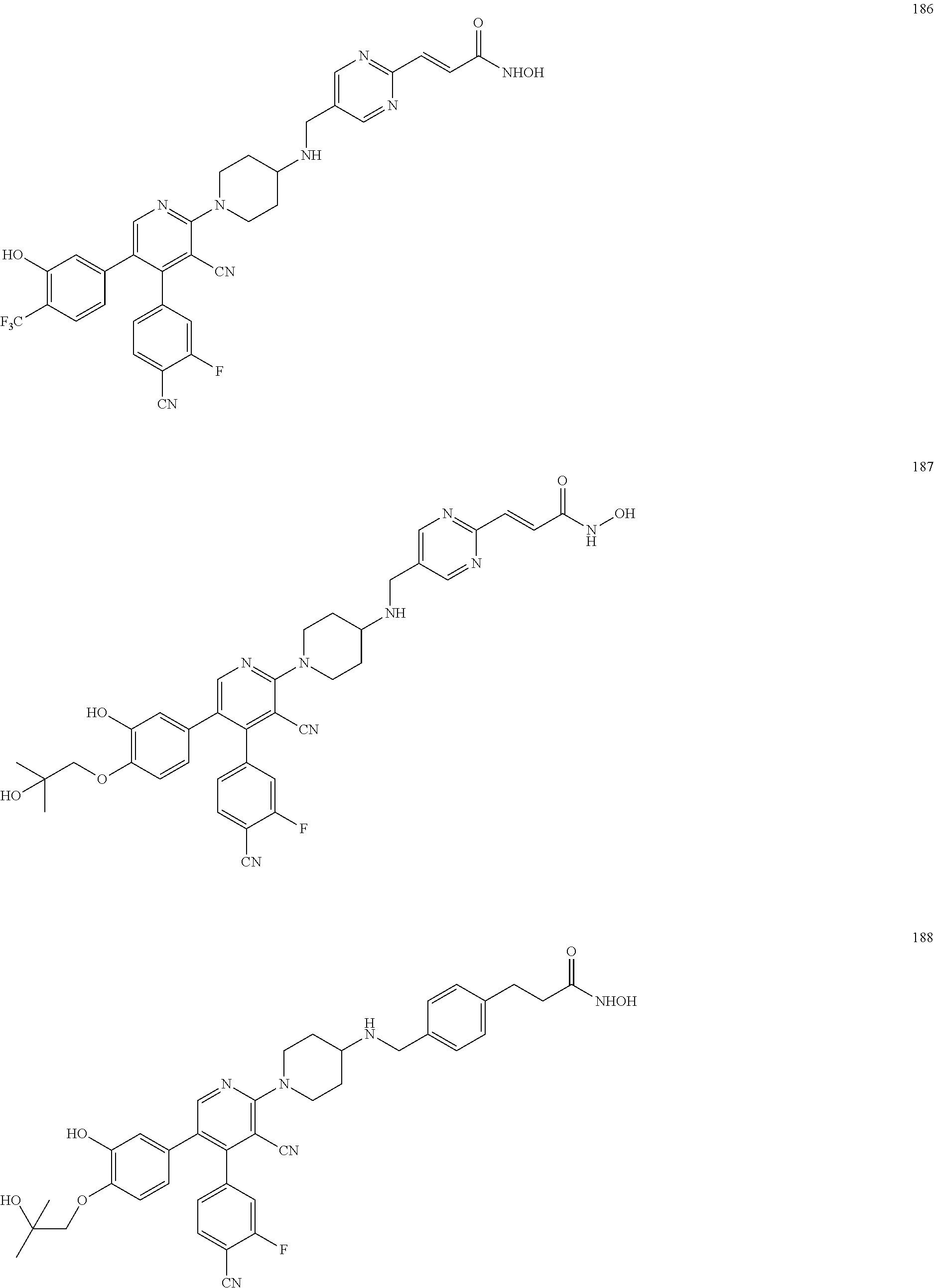

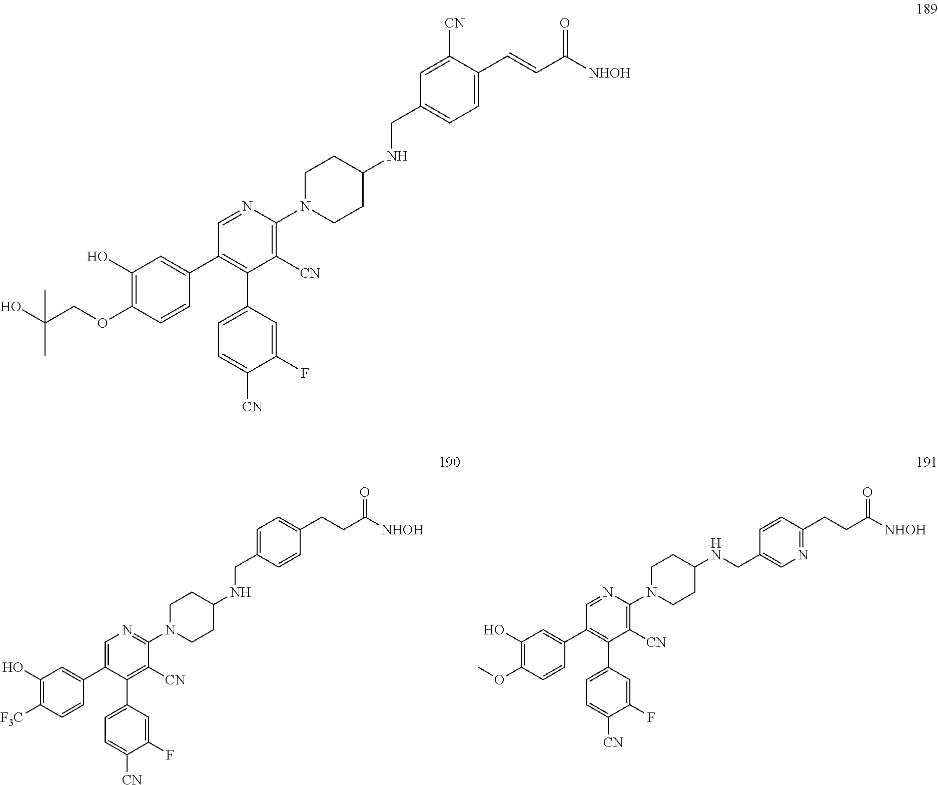

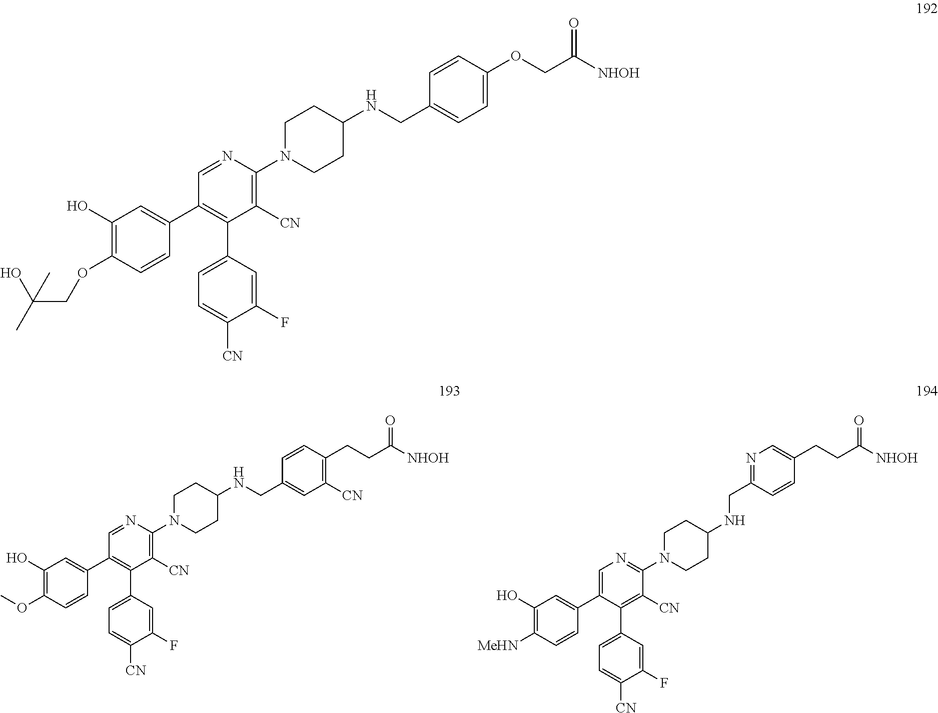

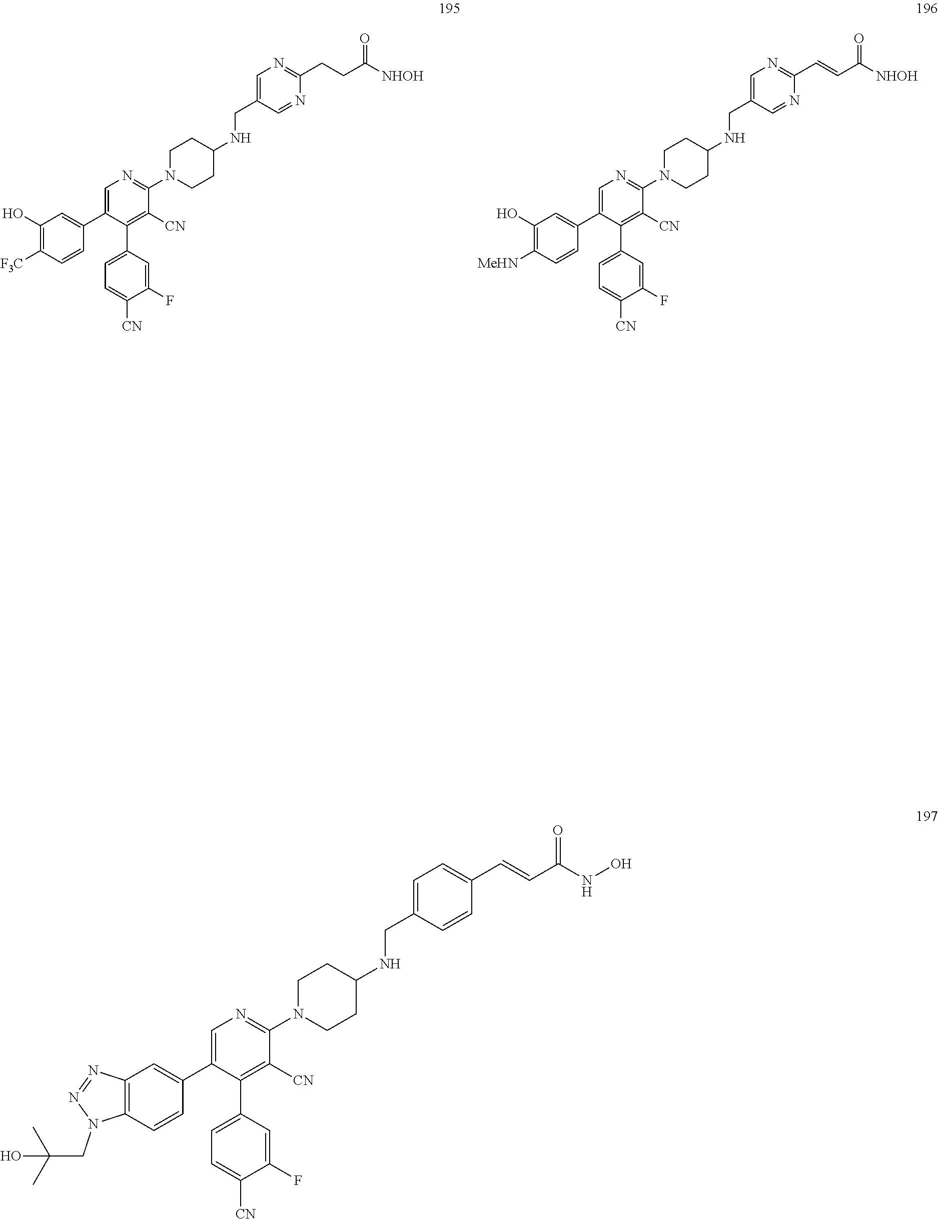

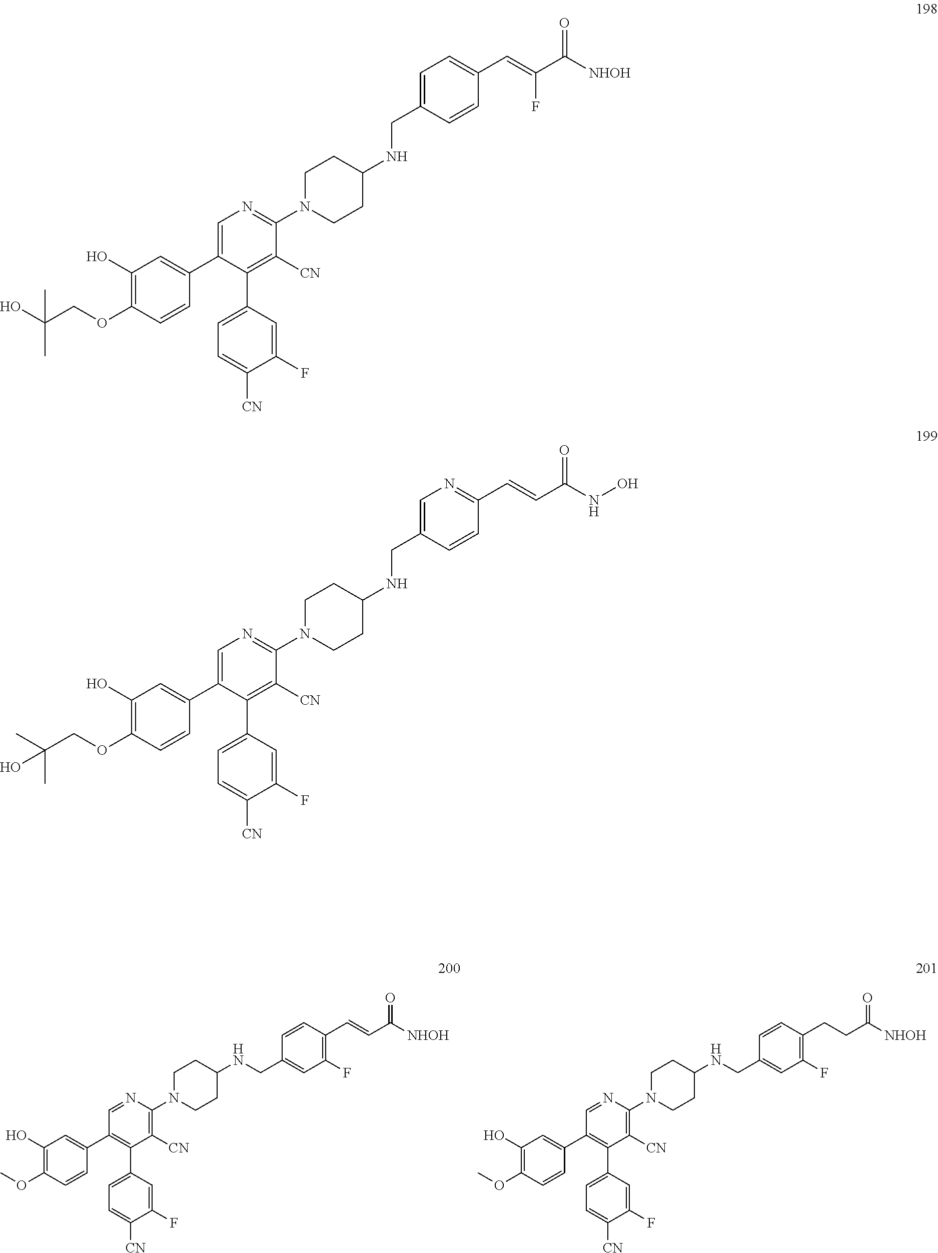

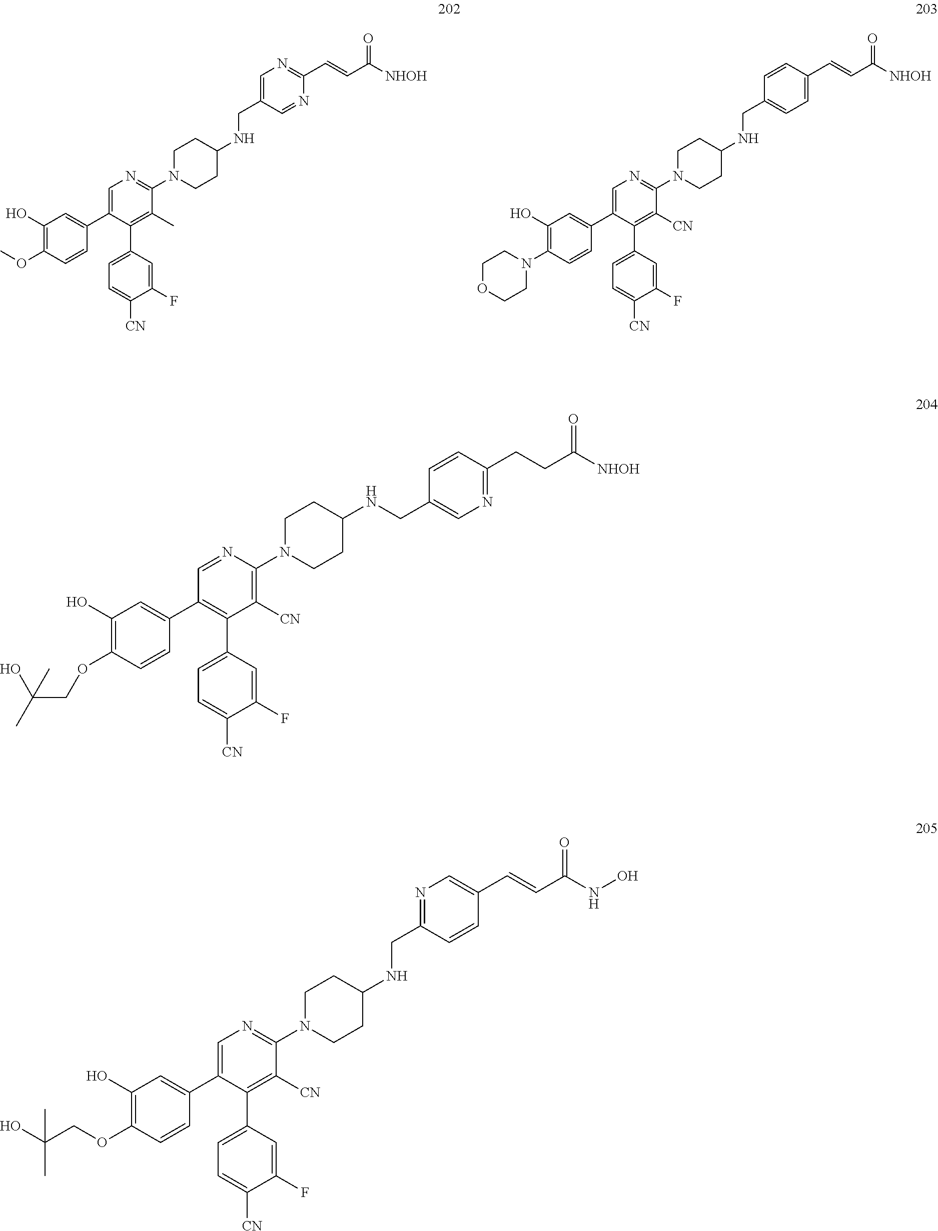

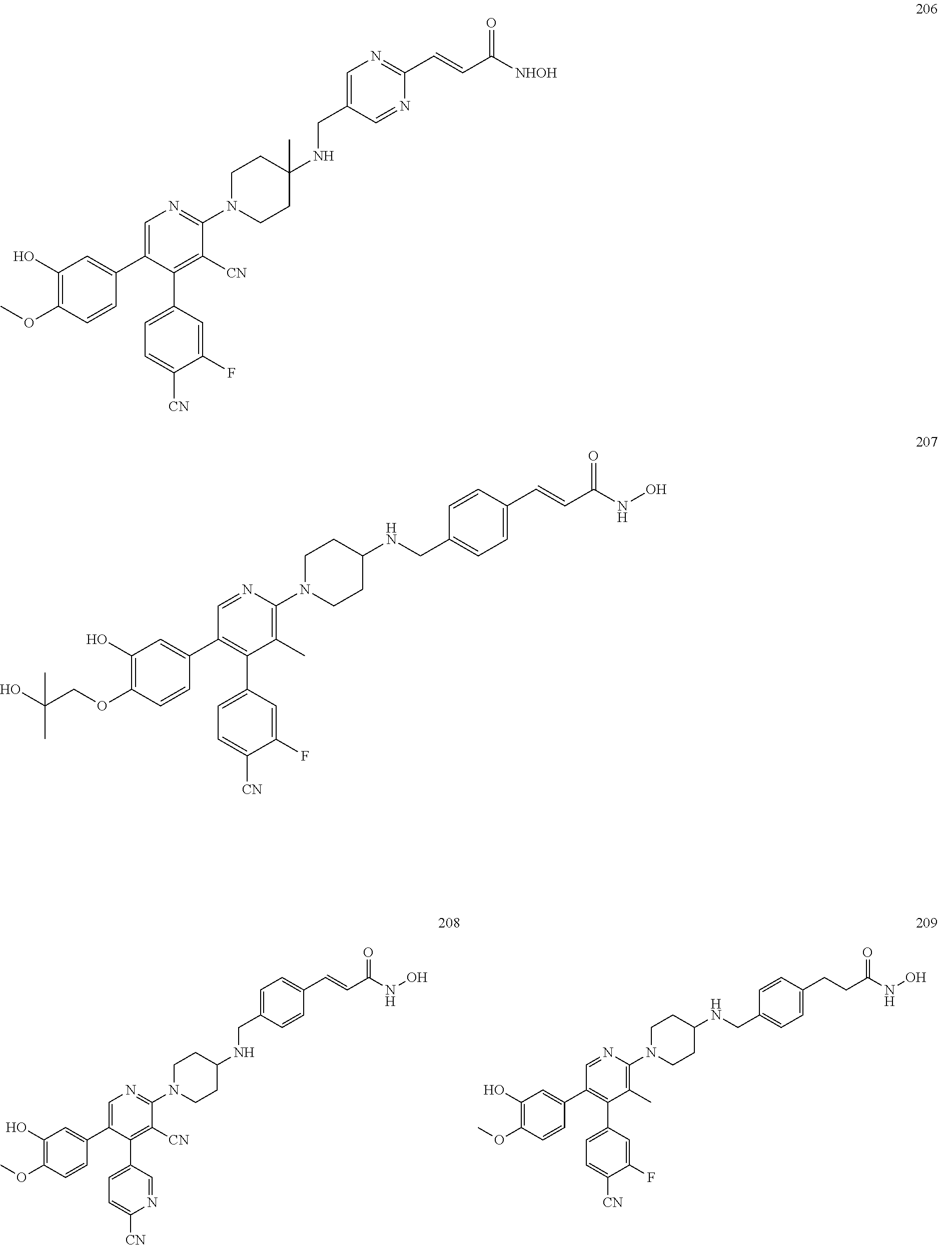

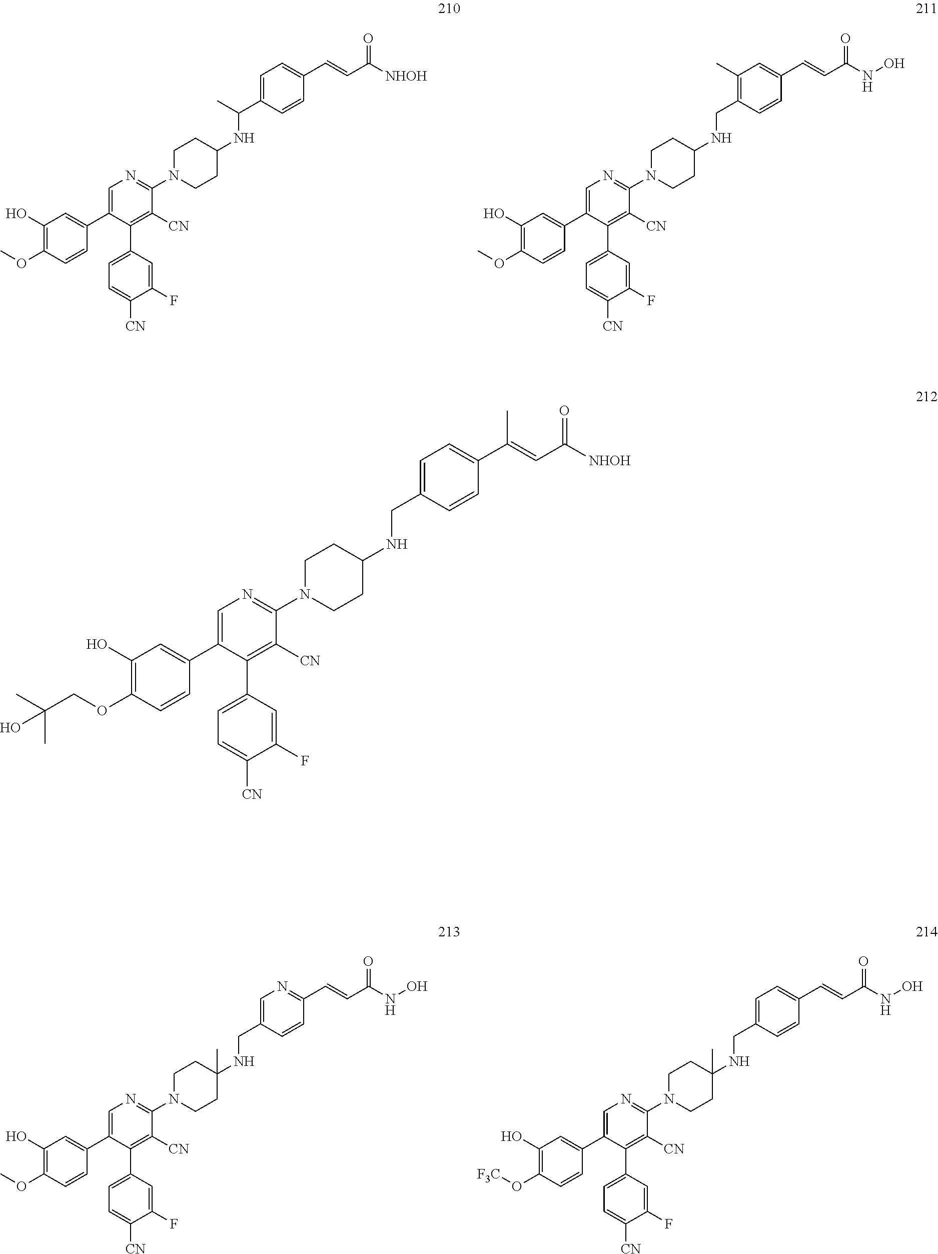

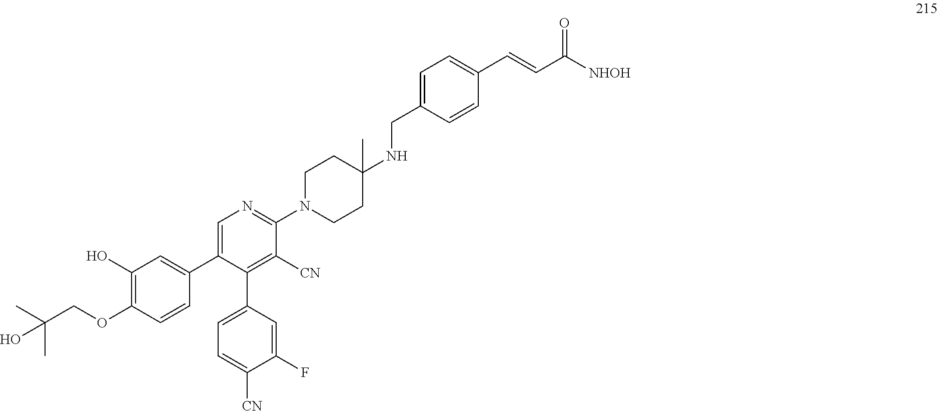

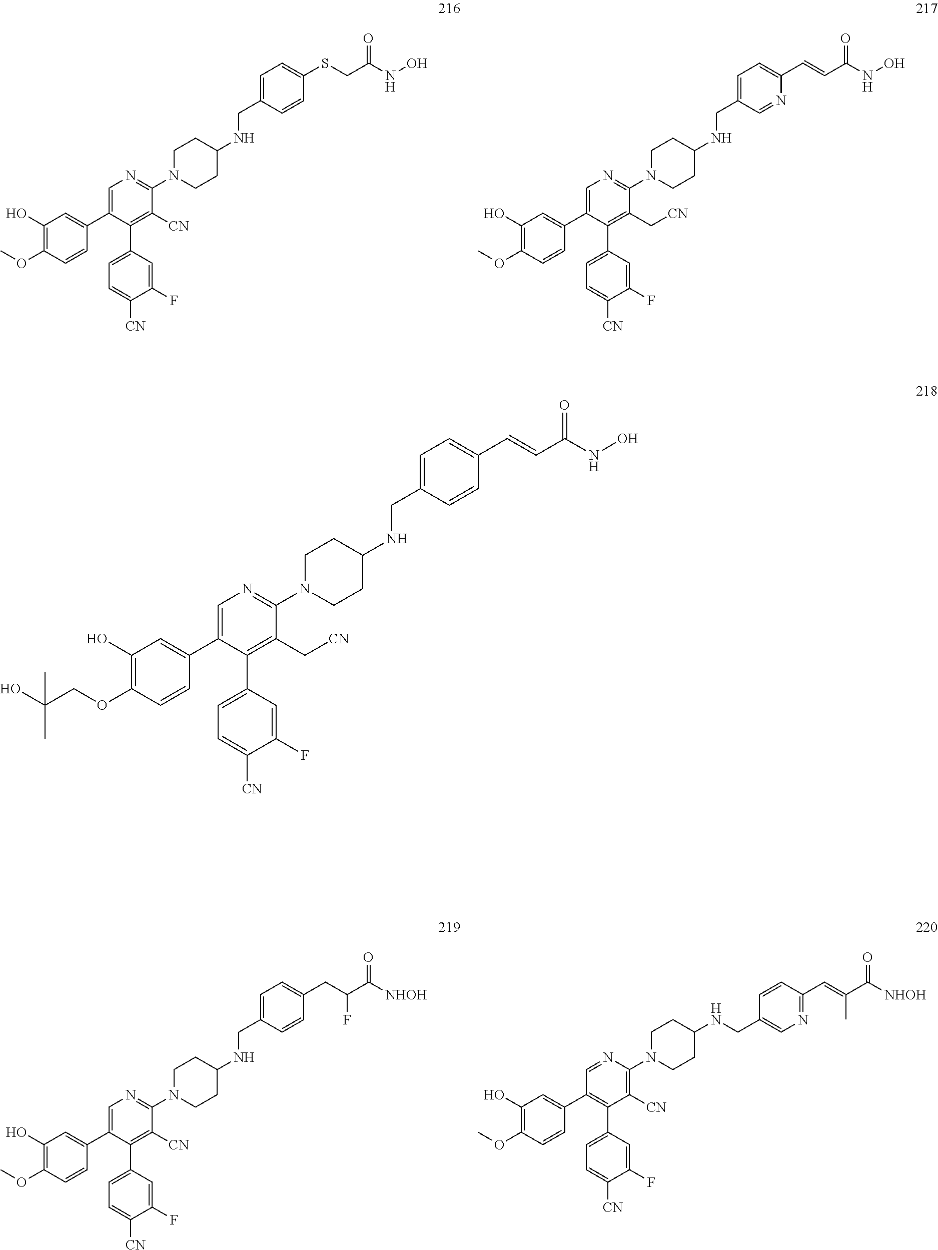

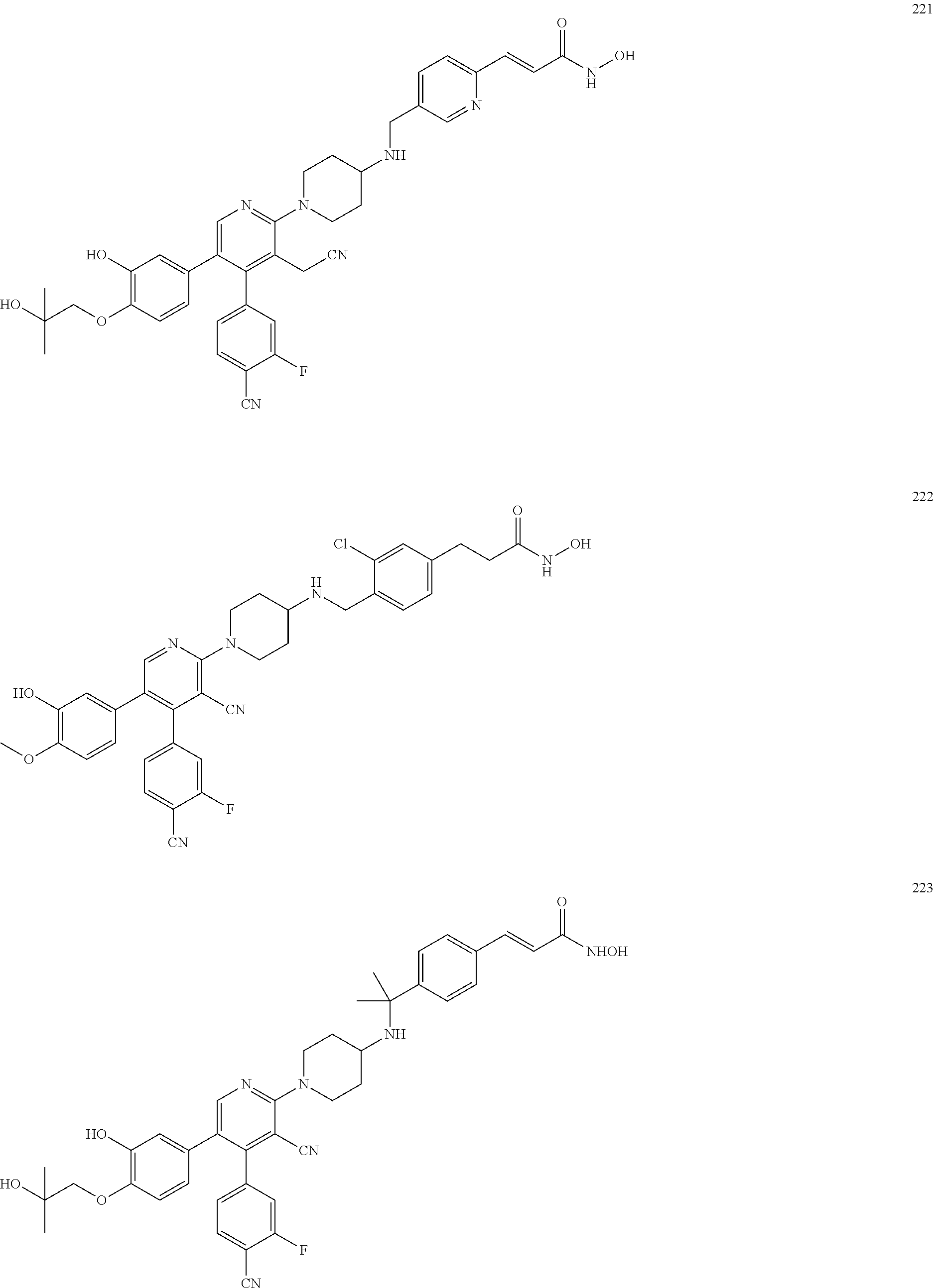

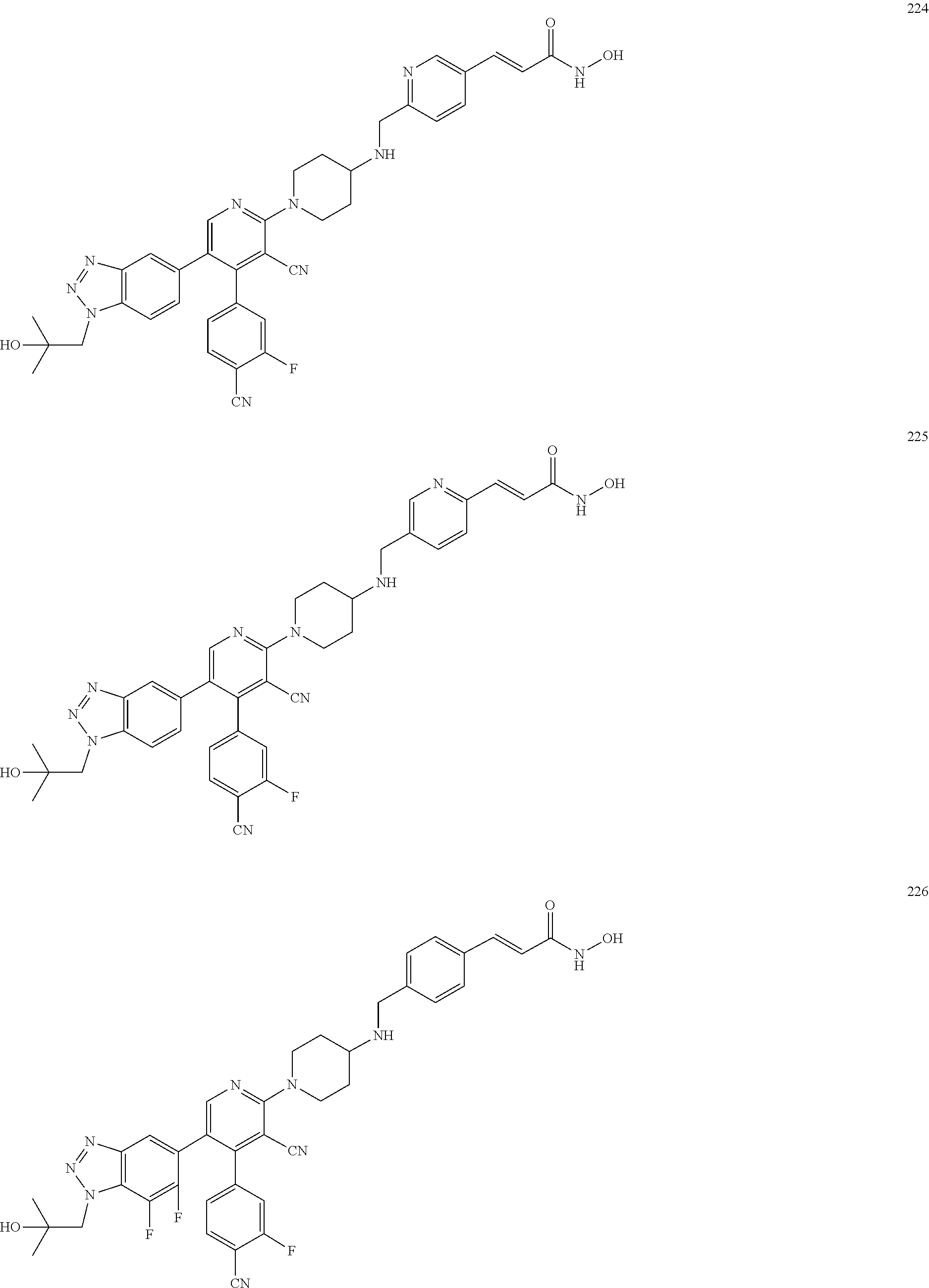 [SMILES: CC(C)(O)Cn1nnc2cc(-c3cnc(N4CCC(NCc5ccc(/C=C/C(=O)NO)cn5)CC4)c(C#N)c3-c3ccc(C#N)c(F)c3)ccc21 ; CC(C)(O)Cn1nnc2cc(-c3cnc(N4CCC(NCc5ccc(/C=C/C(=O)NO)nc5)CC4)c(C#N)c3-c3ccc(C#N)c(F)c3)ccc21 ; CC(C)(O)Cn1nnc2cc(-c3cnc(N4CCC(NCc5ccc(/C=C/C(=O)NO)cc5)CC4)c(C#N)c3-c3ccc(C#N)c(F)c3)c(F)c(F)c21]

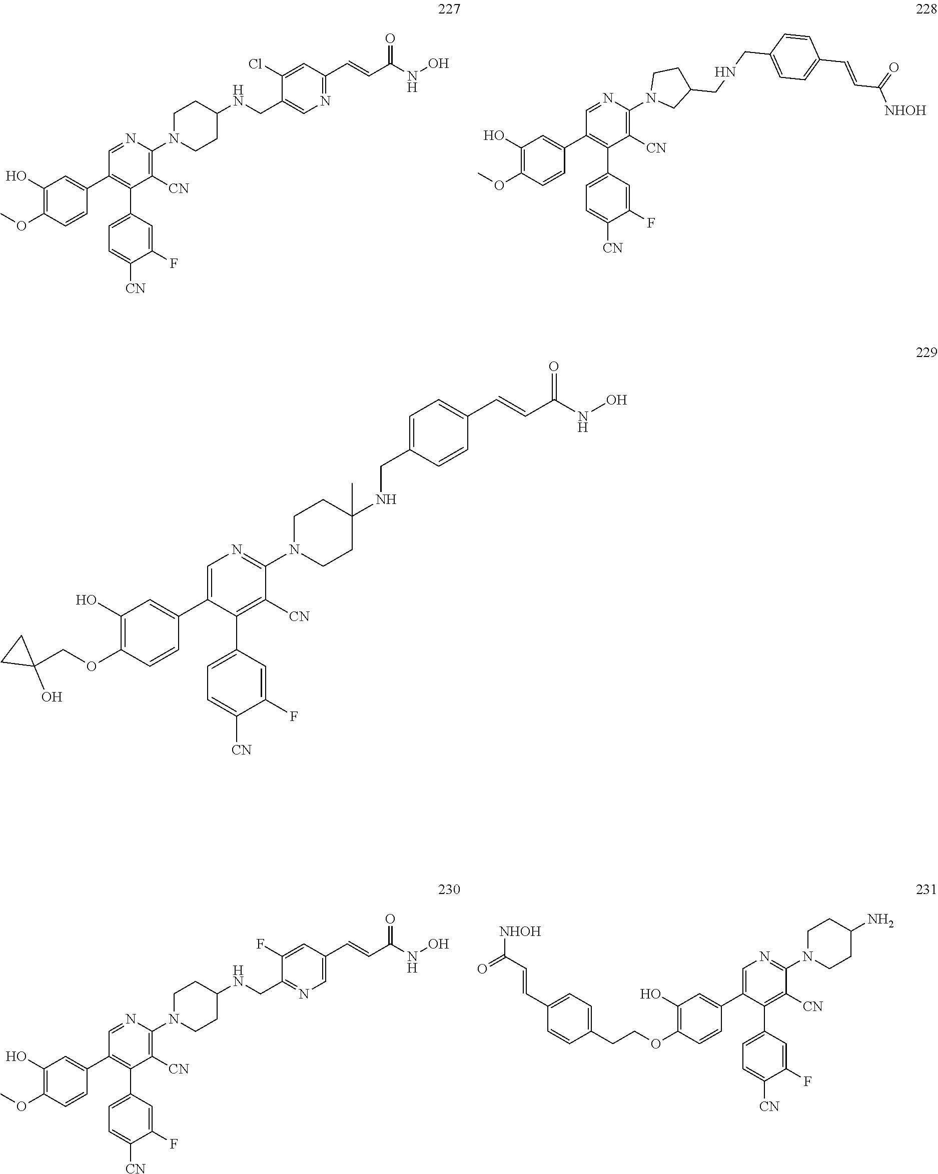

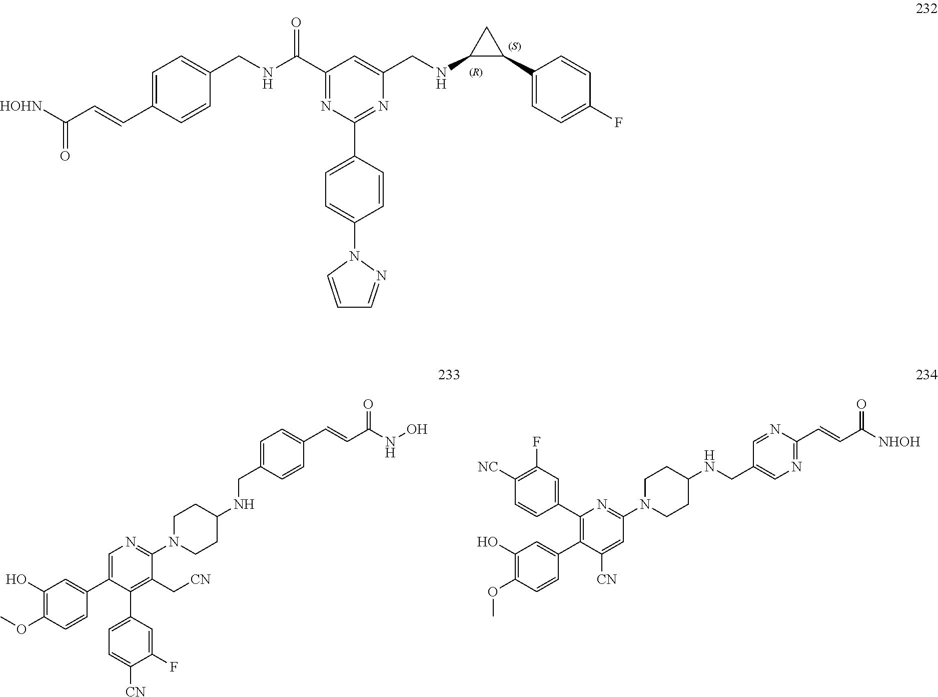

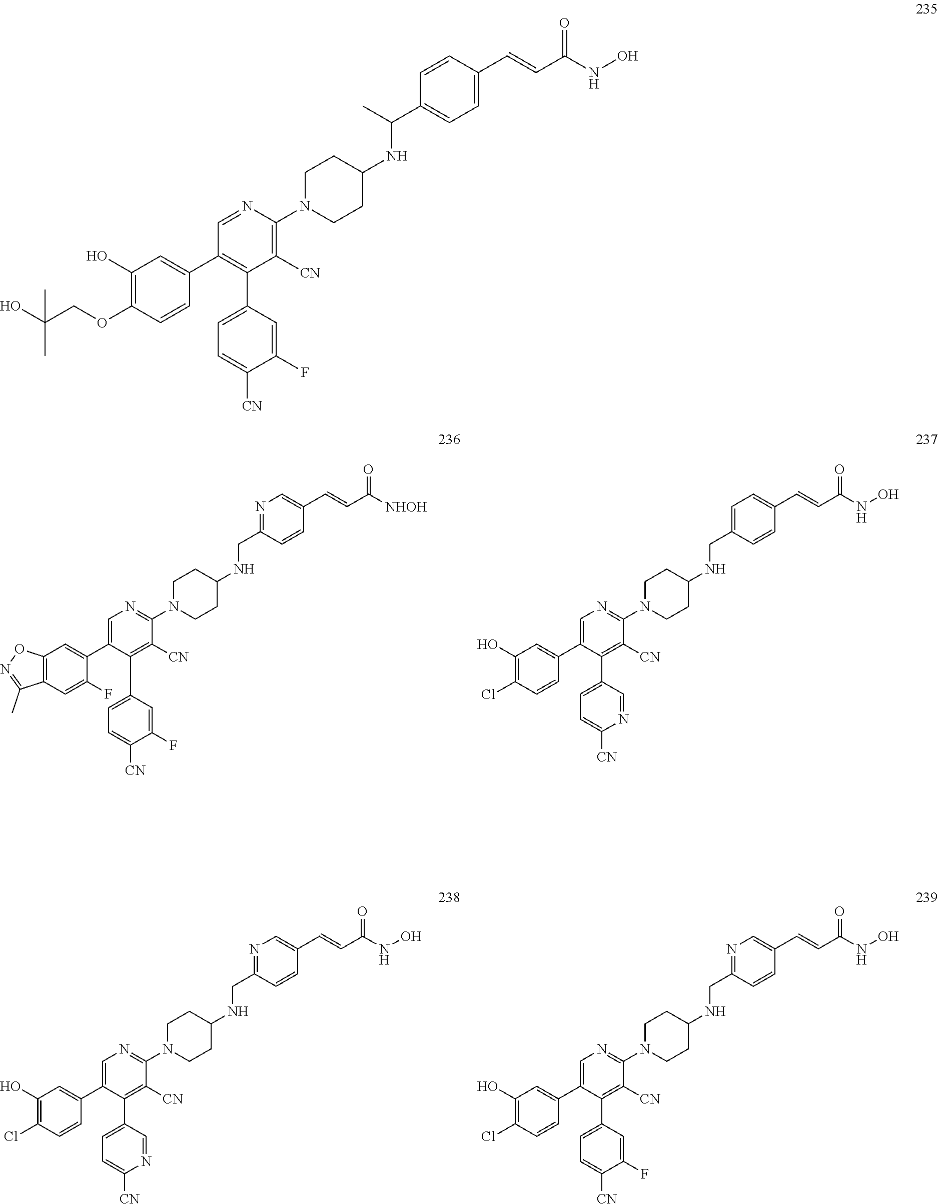

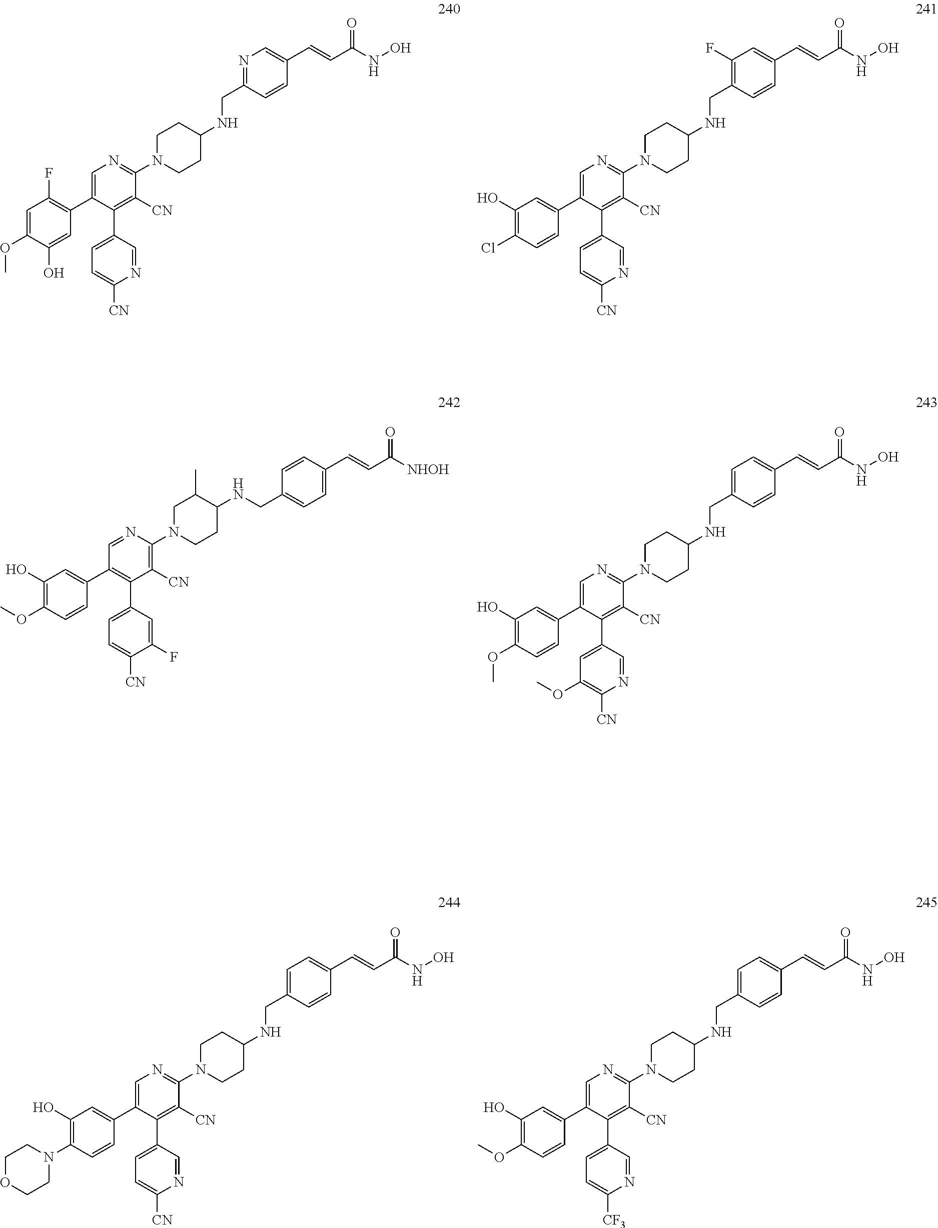

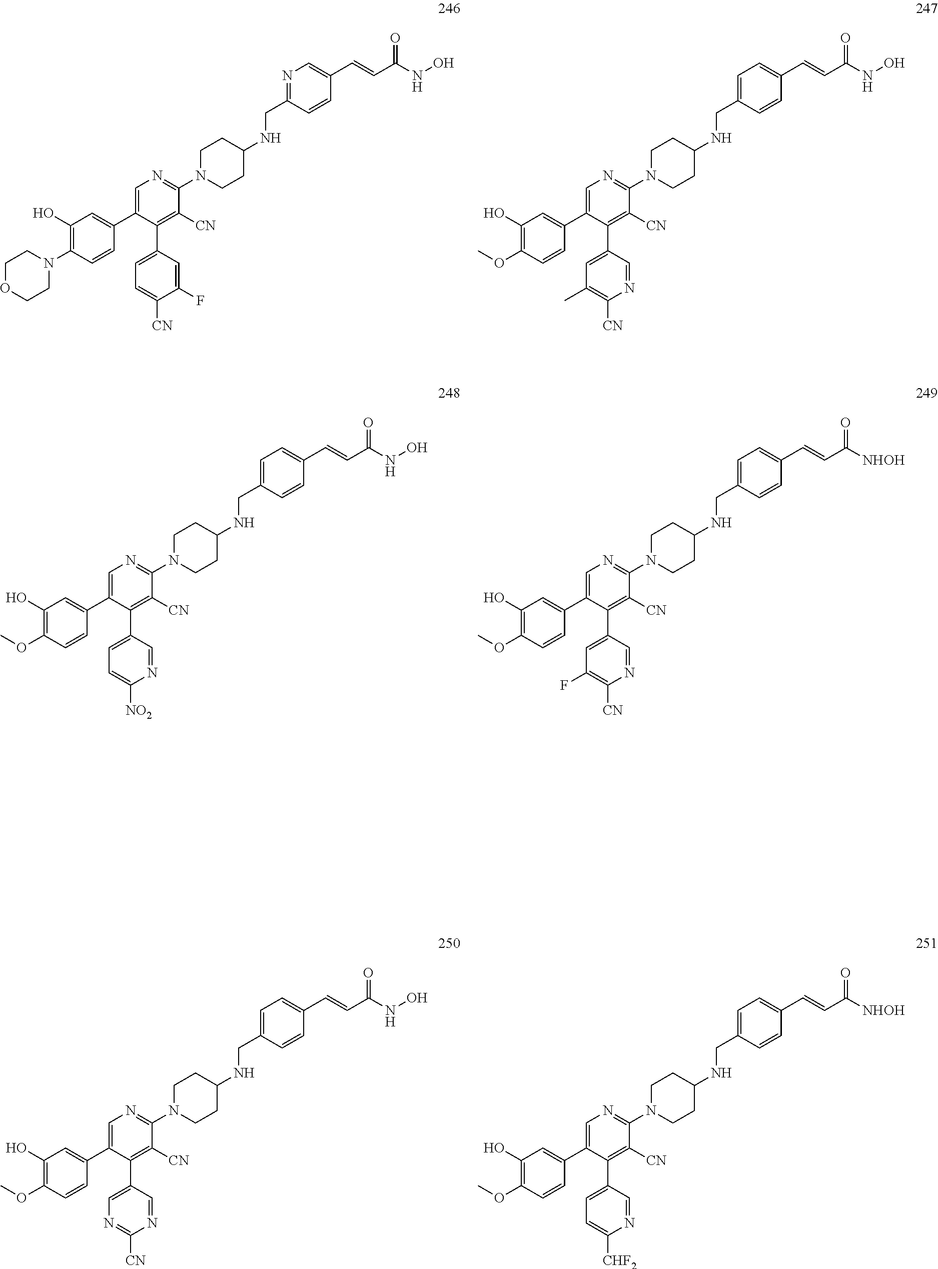

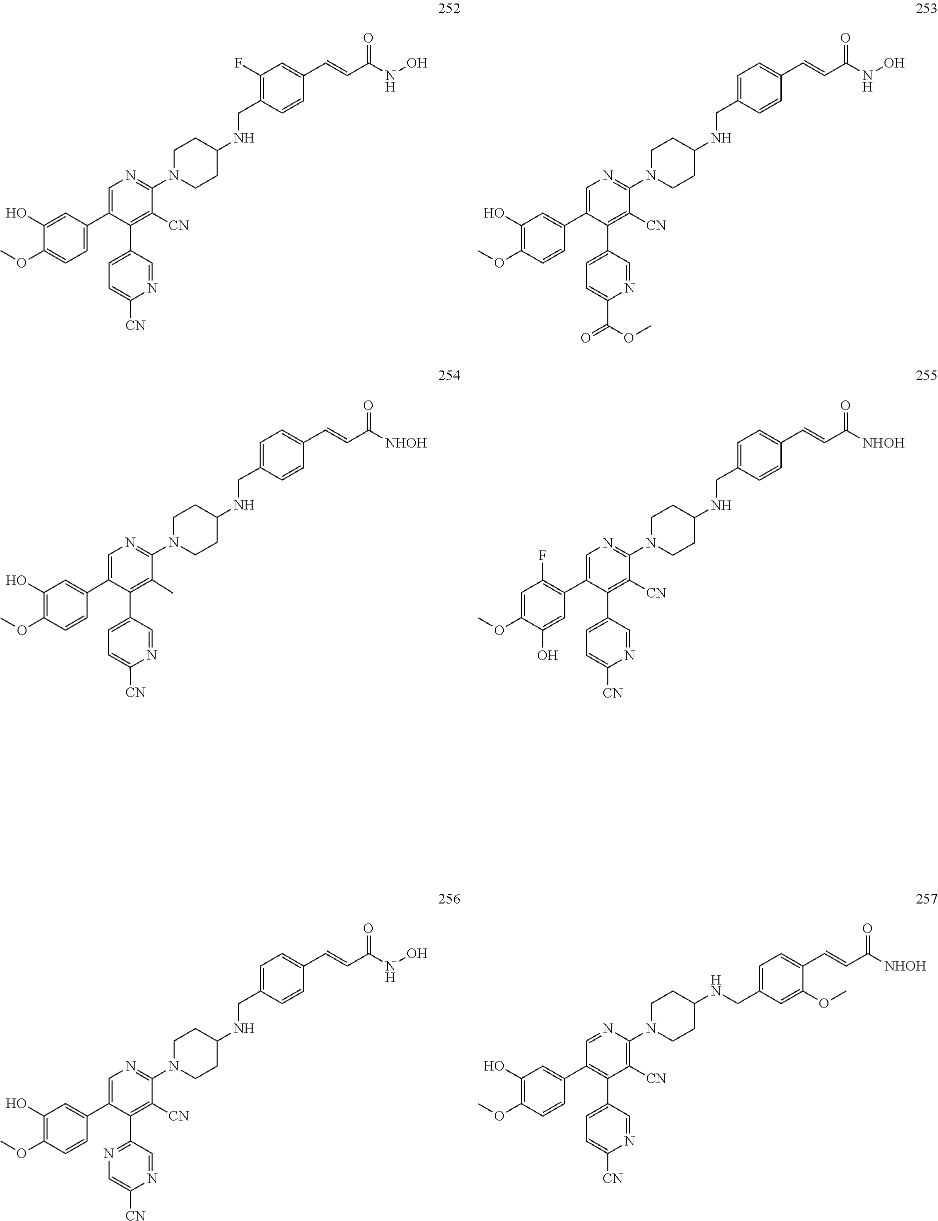

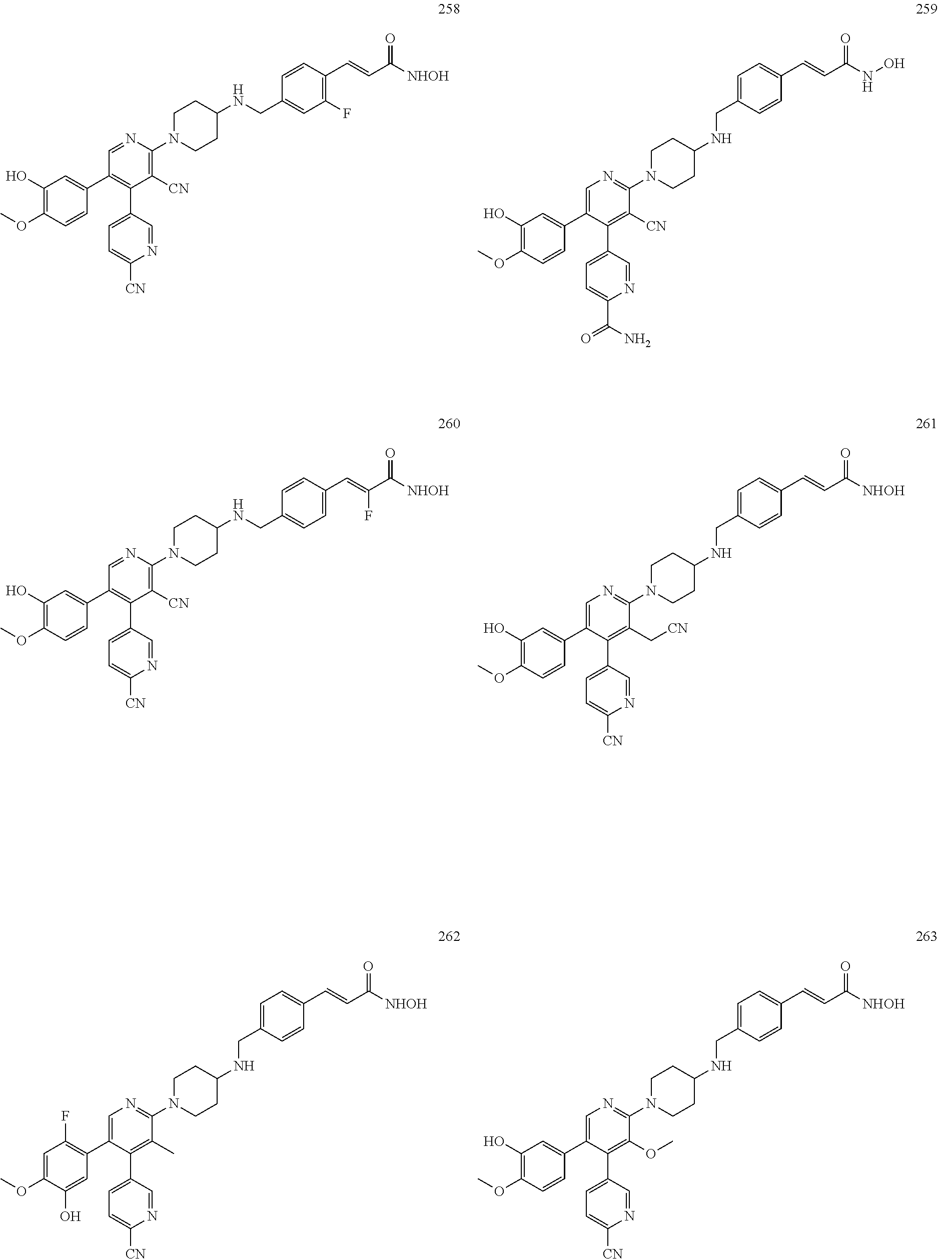

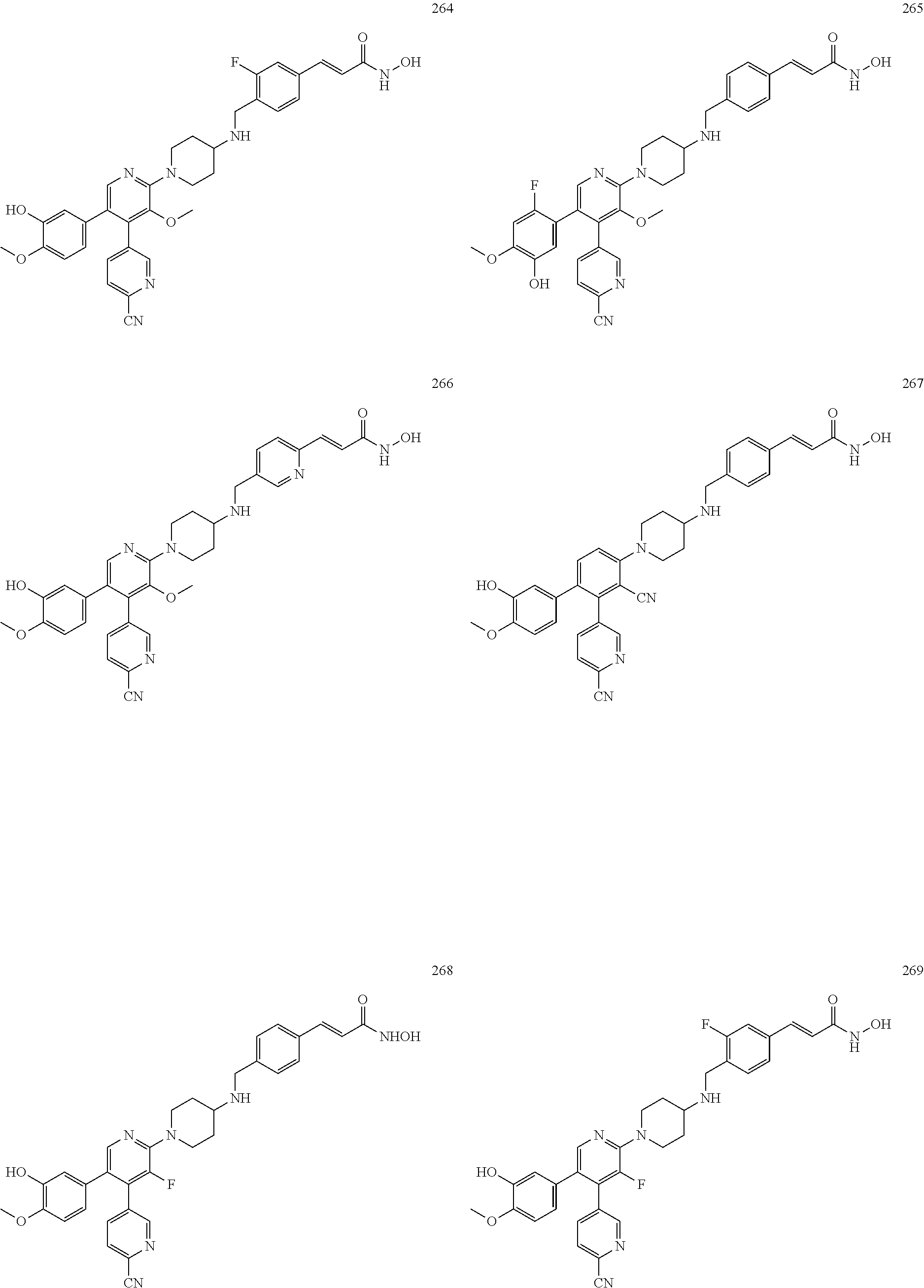

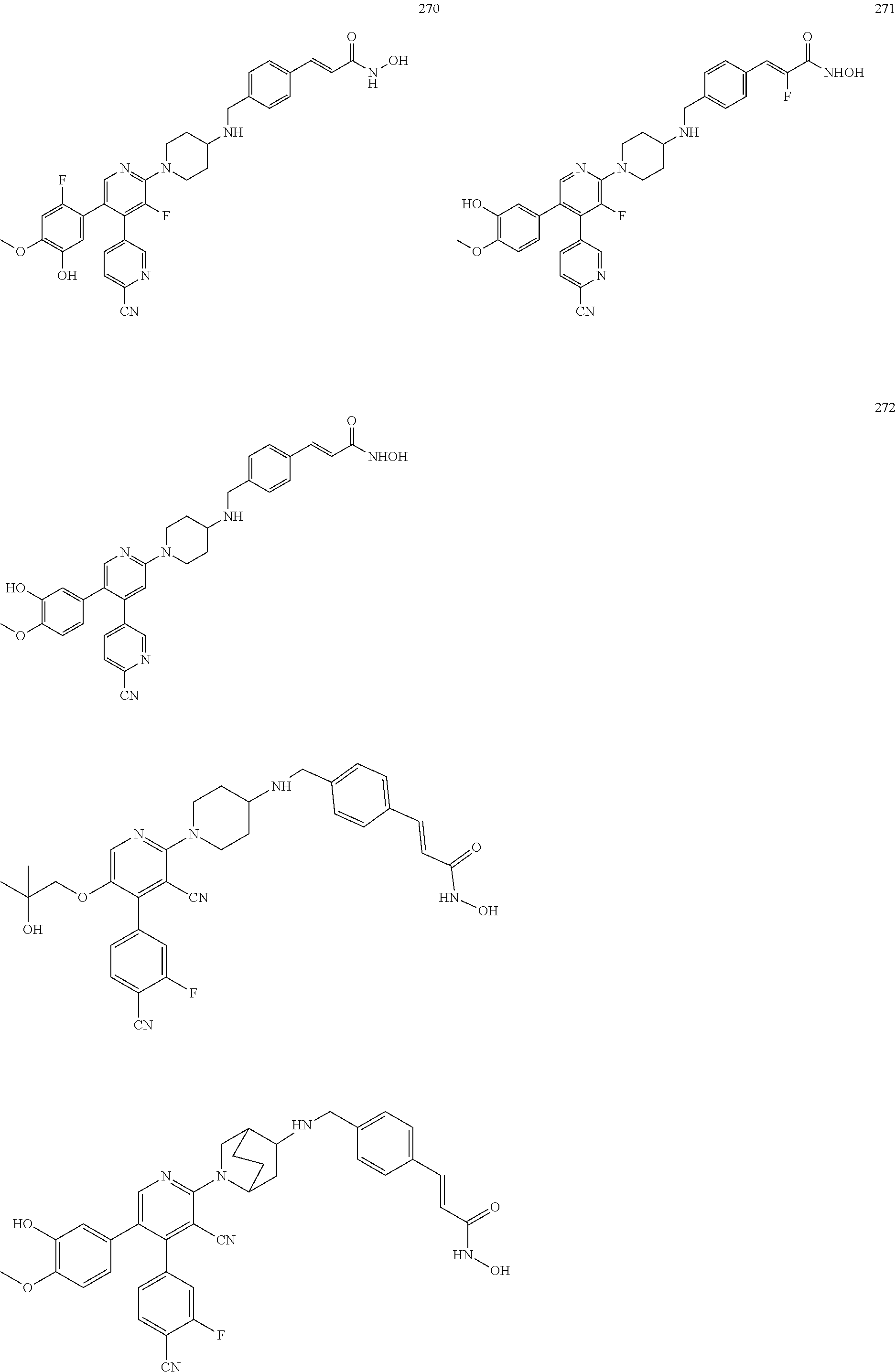

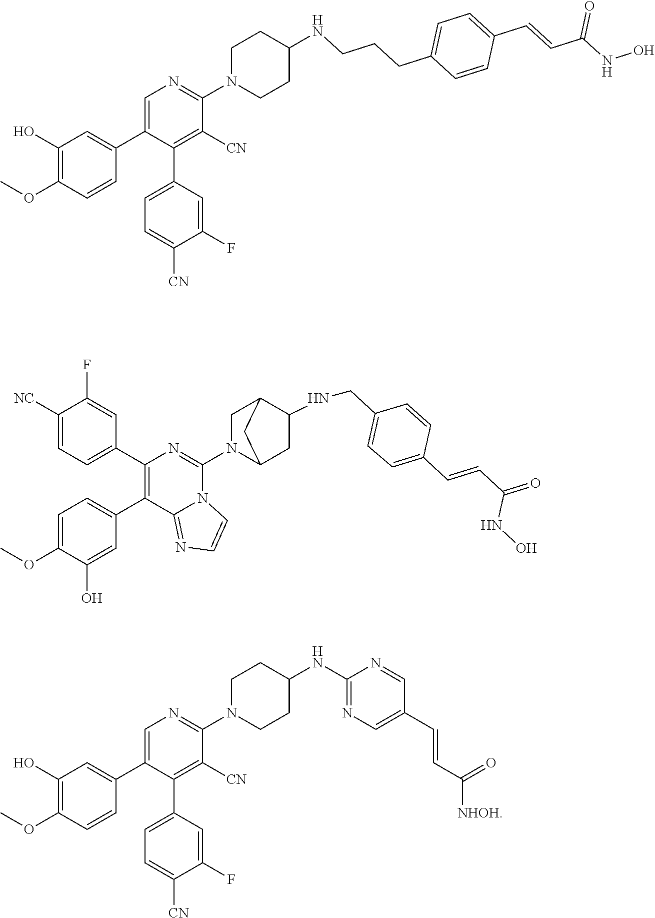

The present disclosure provides a pharmaceutical composition wherein the active ingredient comprising one or a combination of two or more compounds or tautomers, stereoisomers, solvates, metabolites, isotopically-labeled compounds, pharmaceutically acceptable salts or co-crystals thereof mentioned above.

The present disclosure provides a use or method of the compound or tautomer, stereoisomer, solvate, metabolite, isotopically-labeled compound, pharmaceutically acceptable salt or co-crystal thereof mentioned above for preventing and treating the disease mediated respectively or synergistically by LSD1 and/or HDAC.

The use or method comprising the step of administering to a patient in need thereof a therapeutically effective amount of the compound or tautomer, stereoisomer, solvate, metabolite, isotopically-labeled compound, pharmaceutically acceptable salt or co-crystal thereof mentioned above.

In one aspect of the present disclosure, the HDAC enzyme comprises, but is not limited to isoforms of HDAC1, HDAC2, HDAC3, HDAC4, HDAC5, HDAC6, HDAC7, HDAC8 etc., preferably HDAC1, HDAC8 isoforms, further preferably HDAC1 isoform.

The present disclosure provides a use of the compound or tautomer, stereoisomer, solvate, metabolite, isotopically-labeled compound, pharmaceutically acceptable salt or co-crystal thereof mentioned above in the manufacture of a medicament for treating the disease mediated by LSD1 and/or HDAC. In one aspect of the present disclosure, it is the use in the manufacture of a medicament for treating the disease mediated by one or more of LSD1, HDAC.

In one aspect of the present disclosure, the disease is cancer or autoimmune disease.

In one aspect of the present disclosure, the cancer is selected from: non-small cell lung cancer, small cell lung cancer, pancreatic cancer, ovarian cancer, bladder cancer, prostate cancer, chronic myeloid leukemia, colorectal cancer, brain cancer, liver cancer, kidney cancer, gastric cancer, breast cancer, triple negative breast cancer, skin cancer, melanoma, head and neck cancer, bone cancer, cervical cancer, pelvic cancer, vaginal cancer, oral cancer, lymphoma, blood cancer, esophageal cancer, urethral cancer, nasal cavity cancer.

The present disclosure provides a use of the compound or tautomer, stereoisomer, solvate, metabolite, isotopically-labeled compound, pharmaceutically acceptable salt or co-crystal thereof mentioned above, for preventing and treating the disease mediated respectively or synergistically by LSD1 protein and/or HDAC1 protein, LSD1 protein and/or HDAC8 protein.

In one aspect of the present disclosure, the disease is mediated by the abnormal activity of the protein mentioned above.

### DETAILED DESCRIPTION OF THE INVENTION

Unless otherwise defined hereinafter, all technical and scientific terms used herein are intended to have the same meaning as commonly understood by those skilled in the art. References to the art as used herein are intended to refer to the art as commonly understood in the art, including those variations or substitutions of equivalent art that are obvious to those skilled in the art. While the following terms are believed to be well understood by those skilled in the art, the following definitions are set forth to better explain the present disclosure.

### I. DEFINITION

The terms “including”, “comprising” or “containing” and other variations thereof herein are inclusive or open-ended and do not exclude other elements or method steps not listed. It should be understood by those skilled in the art that terms such as “comprising” cover the meaning of “consisting of”.

The term “one or more” or the similar expression “at least one” may denote, for example, 1, 2, 3, 4, 5, 6, 7, 8, 9, 10 or more.

The term “aryl” refers to an all-carbon monocyclic or densely cyclic polycyclic aromatic group having a conjugated π-electron system. As used herein, the term “C₆₋₁₀ aryl” refers to an aromatic group containing 6 to 10 carbon atoms, such as phenyl or naphthyl. The aryl may optionally be substituted with one or more suitable substituents, e.g., substituted with cyano (CN), halogen (F, Cl, Br). When the aryl is substituted with more than one substituent, two adjacent substituents may form a 5-6-membered cycloalkyl or a 5-6-membered heterocycloalkyl together with the carbon atoms to which they attach, examples include, but are not limited to,

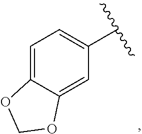 [SMILES: *c1ccc2c(c1)OCO2]

et al.

The term “heteroaryl” refers to a monocyclic, bicyclic or tricyclic aromatic ring system comprising at least one heteroatom which may be the same or different (the heteroatom is, e.g., oxygen, nitrogen or sulphur) and which may additionally, in each case, be benzo-fused. As used herein, the term “C₆₋₁₀ heteroaryl” refers to a monocyclic, bicyclic or tricyclic aromatic ring system having 6-10 ring atoms and containing at least one heteroatom that may be the same or different (the heteroatom is, e.g., oxygen, nitrogen or sulfur). The heteroaryl may optionally be substituted with one or more suitable substituents, e.g., substituted with cyano (CN), halogen (F, Cl, Br). Examples include, but are not limited to,

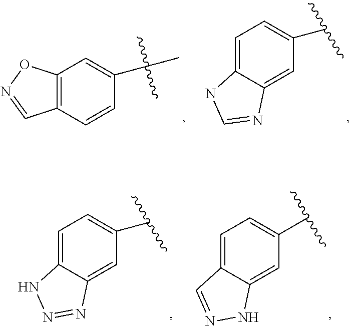 [SMILES: *c1ccc2cnoc2c1 ; *c1ccc2[nH]cnc2c1 ; *c1ccc2[nH]nnc2c1 ; *c1ccc2cn[nH]c2c1]

et al.

The term “cycloalkyl” refers to a saturated monocyclic or polycyclic (e.g., bicyclic) hydrocarbon ring (e.g., monocyclic ring such as cyclopropyl, cyclobutyl, cyclopentyl, cyclohexyl, or bicyclic ring including spirocyclic, fused, or bridged systems (e.g., bicyclo[2.2.1]heptyl, et al). As used herein, the term “C₃₋₆ cycloalkyl” refers to a saturated monocyclic or polycyclic (e.g., bicyclic) hydrocarbon ring (e.g., cyclopropyl, cyclobutyl, cyclopentyl, cyclohexyl) having 3 to 6 ring-forming carbon atoms. The cycloalkyl may optionally be substituted with one or more suitable substituents.

The term “heterocycloalkyl” refers to a saturated monocyclic or polycyclic (e.g., bicyclic) group having 2, 3, 4, 5, 6, 7, 8, 9, or 10 carbon atoms and one or more heteroatoms in the ring; the heterocycloalkyl group may be connected to the rest of the molecule by any one of the carbon atoms or by a heteroatom. As used herein, the term C₃₋₁₀ heterocycloalkyl is a saturated monocyclic or polycyclic (e.g., bicyclic) group having 3-10 ring-forming carbon atoms in the ring and containing at least one heteroatom which may be the same or different (the heteroatom is, e.g., oxygen, nitrogen or sulfur). The heterocycloalkyl may optionally be substituted with one or more suitable substituents.

The term “heterocycloalkenyl” refers to a class of cycloalkenyl groups as defined above in which at least one of the ring-forming carbon atoms is replaced by a heteroatom, e.g., nitrogen, oxygen, or sulfur. Examples of C₃₋₁₀ heterocycloalkenyl include, but are not limited to, tetrahydropyridine, dihydropyran, dihydrofuran, pyrrolizidine, et al, and can be a monocyclic or multicyclic (e.g., bicyclic) group. The heterocyclicalkenyl may optionally be substituted with one or more suitable substituents.

The term “halogen-substituted” or “halogen” group is defined to include F, Cl, Br or I.

The term “hydroxyl” refers to —OH.

The term “alkyl” is defined as a straight or branched saturated aliphatic hydrocarbon. As used herein, the term “C₁₋₆ alkyl” refers to a straight or branched saturated aliphatic hydrocarbon group having 1, 2, 3, 4, 5 or 6 carbon atoms, e.g., methyl, ethyl, n-propyl, isopropyl, n-butyl, sec-butyl, tert-butyl, n-amyl, n-hexyl, et al.

The term “halogen-substituted alkyl”, when used herein alone or in combination with other groups, refers to an alkyl, as defined above, in which one or more hydrogen atoms are substituted with a halogen. It should be understood by those skilled in the art that when there is more than one halogen substituent, the halogens may be the same or different and may be located on the same or different C atoms. As used herein, the term “halogen-substituted C₁₋₆ alkyl” refers to a C₁₋₆ alkyl group in which one or more hydrogen atoms have been replaced by a halogen, e.g, trifluoromethyl.

The term “hydroxyl-substituted alkyl” means an alkyl as defined above in which one or more hydrogen atoms have been replaced by hydroxyl. As used herein, the term “hydroxyl-substituted C₁₋₆ alkyl” means one or more hydrogen atoms of the C₁₋₆ alkyl have been replaced by hydroxyl, e.g,

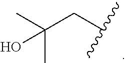 [SMILES: *CC(C)(C)O]

The term “alkoxy” refers to an oxygen atom attached to an “alkyl” as defined above, i.e., an “alkoxy” group can be defined as —OR, where R is an alkyl as defined above. As used herein, examples of the term “C₁₋₆ alkoxy” include, but are not limited to, methoxy, ethoxy, n-propoxy, isopropoxy, n-butoxy, sec-butoxy, tert-butoxy, n-pentyloxy, n-hexyloxy, et al.

The term “halogen-substituted alkoxy” refers to the alkoxy as defined above in which one or more hydrogen atoms are substituted with a halogen. It should be understood by those skilled in the art that when there is more than one halogen substituent, the halogens can be the same or different and can be located on the same or different C atoms. As used herein, the term “halogen-substituted C₁₋₆ alkoxy” refers to one or more hydrogen atoms of the C₁₋₆ alkoxy have been replaced by a halogen, e.g, difluoromethoxy, trifluoromethoxy, et al.

The term “alkenyl” refers to a straight or branched aliphatic hydrocarbon group containing at least one carbon-carbon double bond. The double bond may be present as an E or Z isomer. The double bond may be located at any possible position in the hydrocarbon chain. As used herein, the term “C₂₋₆ alkenyl” refers to an alkenyl containing from 2 to 6 carbon atoms, e.g, vinyl, propenyl, butenyl, butadienyl, pentenyl, pentadienyl, hexenyl, hexadienyl, et al. The alkenyl may optionally be substituted with one or more suitable substituents.

The term “alkynyl” refers to a straight or branched aliphatic hydrocarbon group containing at least one C═C triple bond. The triple bond may be located at any possible position in the hydrocarbon chain. As used herein, the term “C₂₋₆ alkynyl” refers to an alkynyl containing from 2 to 6 carbon atoms, e.g, ethynyl, propynyl, butynyl, pentynyl, hexynyl, et al. The alkynyl may optionally be substituted with one or more suitable substituents.

The term “-(aryl or heteroaryl)-alkenyl-” refers to the aryl or heteroaryl attached to the alkenyl as defined above. As used herein, the term “—(C₆₋₁₀ aryl or heteroaryl)-C₂₋₆ alkenyl-” refers to the aryl containing 6 to 10 carbon atoms or C6-10 heteroaryl attached to the alkenyl containing 2 to 6 carbon atoms, e.g,

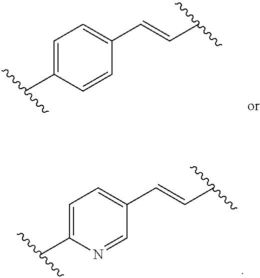 [SMILES: */C=C/c1ccc(*)cc1 ; */C=C/c1ccc(*)nc1]

“Alternatively, one or more alkyl groups of the alkyl may be optionally replaced with one or more groups selected from —C(═O)—, —S(═O)₂— or —NRa—”, refers to one or more alkyl or alkylene fragments of an alkyl group, e.g., C₁₋₁₀ alkyl, is optionally replaced by one or more groups selected from —C(═O)—, —S(═O)₂—, or —NR^{a}—, e.g., when L¹ is —C₁₀ alkyl-C₆₋₁₀ aryl-C₂₋₆ alkenyl-, a segment of the C₁₀ alkyl is substituted with —C(═O)— to afford-C₃ alkyl-C(═O)—C₆ alkyl-—C₆₋₁₀ aryl-C₂₋₆ alkenyl- or —C₉ alkyl-C(═O)—C₆₋₁₀ aryl-C₂₋₆ alkenyl, non-limitingly comprising a methylene group in

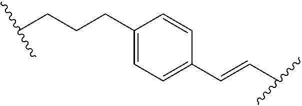 [SMILES: */C=C/c1ccc(CCC*)cc1]

substituted with —NH— to afford

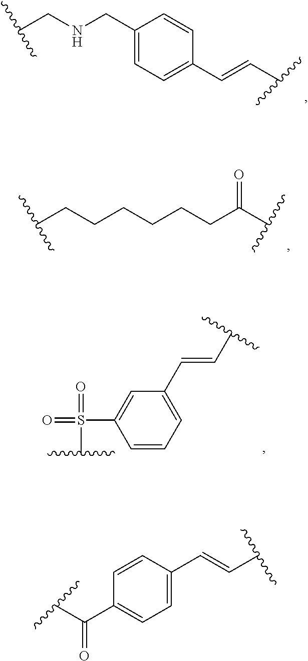 [SMILES: */C=C/c1ccc(CNC*)cc1 ; *CCCCCCC(*)=O ; */C=C/c1cccc(S(*)(=O)=O)c1 ; */C=C/c1ccc(C(*)=O)cc1]

et al.

The term “substituted” refers to one or more (e.g., one, two, three or four) hydrogens on the designated atom are replaced by a selection from the indicated groups, provided that the normal atomic valence of the designated atom in the present case is not exceeded and the substitution results in a stable compound. Combinations of substituents and/or variables are permissible only if such combinations form a stable compound.

The term “hydroxyl-substituted C₁₋₆ alkoxy-C₃₋₆ cycloalkyl” non-limitingly includes

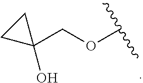 [SMILES: *OCC1(O)CC1]

The term “optionally substituted” refers to optionally substituted with a specific group, atom group or portion.

When a group is described as “optionally substituted with one or more substituents”, the group may be (1) unsubstituted or (2) substituted. If the carbon on a group is described as being optionally substituted with one or more substituents, the one or more hydrogens on the carbon (to the extent of any hydrogens present) may be substituted or unsubstituted individually and/or collectively with independently selected substituents. If the nitrogen on a group is described as optionally substituted with one or more substituents, the one or more hydrogens on the nitrogen (to the extent of any hydrogen present) may each be substituted or unsubstituted with independently selected substituents.

When the bond of the substituent is shown to pass through a bond connecting two atoms in the ring, such a substituent may be bonded to any of the ring-forming atoms in the substitutable ring.

[symbol] indicates a double bond may be present or not present at any position within the ring, which means that it includes various scenarios such as saturated cyclic systems, unsaturated non-aromatic cyclic systems with double bonds, and aromatic cyclic systems.

The compounds of the present disclosure may also comprise one or more (e.g., one, two, three, or four) isotopic replacement.

The term “stereoisomer” refers to an isomer formed due to at least one asymmetric center. In a compound having one or more (e.g., one, two, three, or four) asymmetric centers, racemates, racemic mixtures, mono enantiomers, diastereoisomeric mixtures, and individual diastereoisomers may result. Specific individual molecules may also exist in geometric isomers (cis/trans). Similarly, the compound of the present disclosure may exist in mixtures of two or more structurally different forms in rapid equilibrium (commonly referred to as tautomer). Representative examples of tautomers include keto-enol tautomers, phenol-keto tautomers, nitroso-oxime tautomers, imine-enamine tautomers, et al. The scope of the present application covers all such isomers or mixtures thereof in any ratio (e.g., 60%, 65%, 70%, 75%, 80%, 85%, 90%, 95%, 96%, 97%, 98%, 99%).

Pharmaceutically acceptable salts of the compound of the present disclosure may include acid addition salts and alkali salts of the compound. Suitable acid addition salts are formed from acids that form non-toxic salts.

In some embodiments, pharmaceutically acceptable salts of the compound of the present disclosure, are selected from hydrochloride, formate, trifluoroacetate, et al.

The term “co-crystal” refers to a crystal formed by the combination of an active pharmaceutical ingredient and a co-crystal-forming agent (e.g., a coformer) in the presence of hydrogen bonds or other non-covalent bonds, and more specifically, a co-crystal formed from the compound of formula (I) and a pharmaceutically acceptable coformer.

The term “therapeutically effective amount” refers to a sufficient amount of a drug or agent that is non-toxic but achieves the desired effect. In embodiments of the present disclosure, when treating a patient in accordance with the present disclosure, the amount of a given drug depends on a number of factors, such as the specific dosing regimen, the type of disease or condition and the severity thereof, and the uniqueness (e.g., body weight) of the one to be treated or host to be treated. However, the dose to be administered may be routinely determined by methods known in the art based on the particular surrounding circumstances, including, for example, the specific drug that has been employed, the route of administration, the condition to be treated, and the one to be treated or host to be treated. Typically, for therapeutic use in adults, the administered dose is typically in the range of 0.02-5000 mg/day, such as about 1-1500 mg/day. This desired dose may conveniently be expressed as a single dose, or as divided doses administered concurrently (or over a short period of time) or at appropriate intervals, such as two, three, four or more divided doses per day. It will be appreciated by one of skill in the art that, although the above dosage ranges are given, the specific effective amount may be appropriately adjusted according to the patient's condition and in conjunction with the physician's diagnosis.

### II. EXAMPLES

The following detailed description of the implementation process and the beneficial effects produced by the present disclosure by means of specific examples is intended to help the reader better understand the essence and characteristics of the present disclosure, and is not intended to be a limitation on the implementable scope of the present case.

The structure of the compound of the present disclosure was determined by nuclear magnetic resonance (NMR) or/and mass spectrometry (MS). The NMR chemical shifts (δ) are given in parts per million (ppm). The NMR determinations were made with an AVANCE NEO 400 MHz Bruker instrument, the solvents for the determinations were deuterated dimethyl sulfoxide (DMSO-d₆), deuterated chloroform (CDCl₃), deuterated methanol (CD₃OD) and the internal standard was tetramethylsilane (TMS). MS was determined with an ISQ-EC Thermo Fisher LC-MS instrument. The instrument used for preparative chromatography was a GX-281 Gilson chromatograph. The following separation methods were: separation method 1: Sun Fire Prep C18 OBD™ 5 μm, 30×150 mm Column, 0.04% HCl aqueous solution/acetonitrile; separation method 2: Sun Fire Prep C18 OBD™ 5 μm, 30×150 mm Column, 0.02% TFA aqueous solution/acetonitrile; separation method 3: Sun Fire Prep C18 OBD™ 5 μm, 30×150 mm Column, 0.06% formic acid aqueous solution/acetonitrile; separation method 4: Xbridge Prep C18 OBD™ 5 μm, 30×150 mm Column, 10 mM NH₄HCO₃ aqueous solution/acetonitrile; separation method 5: Xbridge Prep C18 OBD™ 5 μm, 30×150 mm Column, 0.6% NH₃·H₂O aqueous solution/acetonitrile.

The solvents used in the present disclosure are commercially available.

Unless otherwise specified in the Examples, solutions are aqueous solutions.

Unless otherwise specified in the Examples, the temperature for the reaction is room temperature, i.e., 20° C. to 30° C.

The chemical abbreviations involved in the present disclosure have the following meanings:
HATU: O-(7-azabenzotriazol-1-yl)-N,N,N′,N′-tetramethyluronium hexafluorophosphate;
DIPEA: diisopropylethylamine
DMSO: dimethyl sulfoxide;
EA: ethyl acetate;
Pd₂(dba)₃: tris(dibenzylideneacetone)dipalladium;
DCE: dichloroethane;
DMF: N,N-dimethylformamide;
NMP: N-methylpyrrolidone;
TFA: trifluoroacetic acid;
NBS: N-bromosuccinimide;
TfOH: trifluoromethanesulfonic acid;
DPPA: diphenyl phosphate azide.

Positive references are CC-90011 and SAHA (Vorinostat), respectively.

### Example 1

### Preparation of 7-((6-(4-aminopiperidin-1-yl)-2-(4-cyano-3-fluorophenyl)-3-(3-hydroxy-4-methoxyphenyl)pyridin-4-yl)oxy)-N-hydroxyheptanamide hydrochloride

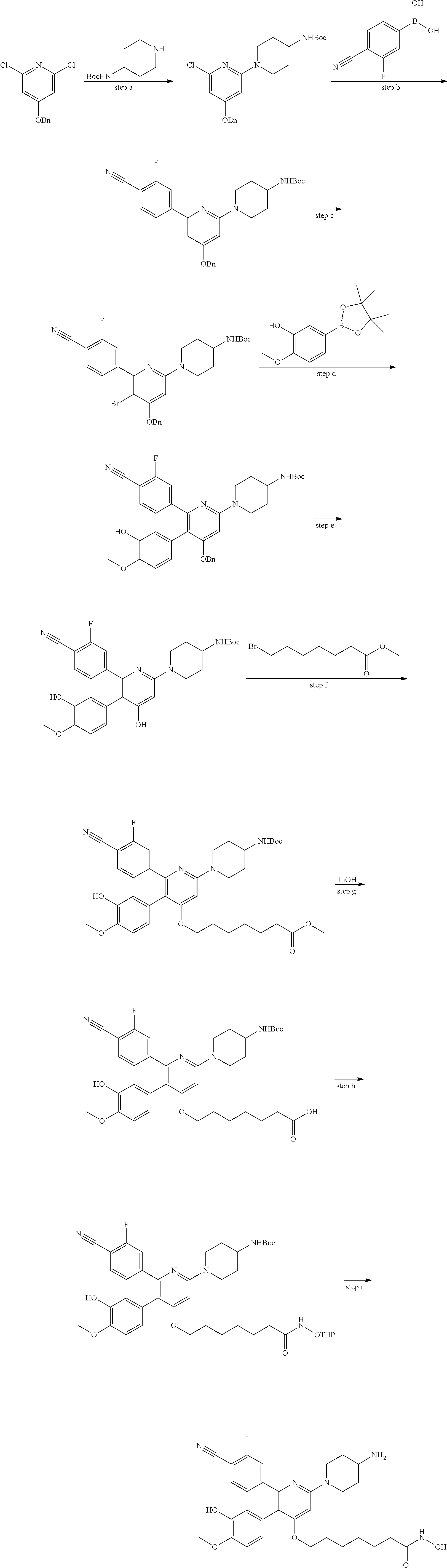

### Step a): preparation of tert-butyl (1-(4-(benzyloxy)-6-chloropyridin-2-yl)piperidin-4-yl)carbamate

4-(Benzyloxy)-2,6-dichloropyridine (1.8 g, 7.1 mmol), tert-butylpiperidine-4-carbamate (1.4 g, 7.1 mmol), and DIPEA (415 mg, 14.2 mg) were dissolved in NMP (20 mL), and the reaction was heated up to 130° C. and reacted for 2 hours. When the reaction was completed as monitored by LC-MS, the reaction liquid was cooled to room temperature, then added with water (50 mL), and extracted with ethyl acetate (30 mL×3). The organic phases were combined, washed with brine (20 mL), dried over anhydrous sodium sulfate, filtered, concentrated in vacuum, and the residue was purified by silica gel chromatography (eluent: petroleum ether/ethyl acetate=2:1) to afford tert-butyl (1-(4-(benzyloxy)-6-chloropyridin-2-yl)piperidin-4-yl)carbamate with a yield of 77.4%.

ESI-MS m/z=418.2 [M+H]⁺.

### Step b): preparation of tert-butyl (1-(4-(benzyloxy)-6-(4-cyano-3-fluorophenyl)pyridin-2-yl)piperidin-4-yl)carbamate

Tert-butyl (1-(4-(benzyloxy)-6-chloropyridin-2-yl)piperidin-4-yl)carbamate (2.3 g, 5.5 mmol), (4-cyano-3-fluorophenyl)boronic acid (909 mg, 5.5 mmol), Cs₂CO₃ (3.5 g, 11.0 mmol), and Pd(dppf)Cl₂ (77.2 mg, 0.11 mmol) were dissolved in 1,4-dioxane (40 mL) and water (4 mL) was added, the reaction mixture was purged with nitrogen three times, then heated to 100° C. and reacted for 2 hours, and the reaction was completed as indicated by LC-MS. After cooling to room temperature, water (50 mL) was added, and the mixture was extracted with ethyl acetate (30 mL×3). The organic phases were combined, washed with brine (20 mL), dried over anhydrous sodium sulfate, filtered, and concentrated under vacuum. The residue was purified by silica gel chromatography (eluent: petroleum ether/ethyl acetate=1:1) to afford tert-butyl (1-(4-(benzyloxy)-6-(4-cyano-3-fluorophenyl)pyridin-2-yl)piperidin-4-yl)carbamate with a yield of 76.0%.

ESI-MS m/z=503.2 [M+H]⁺.

### Step c): preparation of tert-butyl (1-(4-(benzyloxy)-5-bromo-6-(4-cyano-3-fluorophenyl)pyridin-2-yl)piperidin-4-yl)carbamate

Tert-butyl (1-(4-(benzyloxy)-6-(4-cyano-3-fluorophenyl)pyridin-2-yl)piperidin-4-yl)carbamate (1.8 g, 3.58 mmol) and NBS (687 mg, 2.41 mmol) were dissolved in DMF (30 mL) and stirred at room temperature for 2 hours, and the reaction was completed as indicated by LC-MS. Water (80 mL) was added, and the mixture was extracted with ethyl acetate (40 mL×3). The organic layers were combined, washed with brine (20 mL), dried over anhydrous sodium sulfate, filtered, and concentrated under vacuum. The residue was then purified by silica gel chromatography (eluent: petroleum ether/ethyl acetate=1:2) to afford tert-butyl (1-(4-(benzyloxy)-5-bromo-6-(4-cyano-3-fluorophenyl)pyridin-2-yl)piperidin-4-yl)carbamate with a yield of 81.0%.

ESI-MS m/z=581.2 [M+H]⁺.

### Step d): preparation of tert-butyl (1-(4-(benzyloxy)-6-(4-cyano-3-fluorophenyl)-5-(3-hydroxy-4-methoxyphenyl)pyridin-2-yl)piperidin-4-yl)carbamate

Tert-butyl (1-(4-(benzyloxy)-5-bromo-6-(4-cyano-3-fluorophenyl)pyridin-2-yl)piperidin-4-yl)carbamate (1.7 g, 2.9 mmol), 2-methoxy-5-(4,4,5,5-tetramethyl-1,3,2-dioxaborolan-2-yl)phenol (498 mg, 2.9 mmol), Cs₂CO₃ (1.9 g, 5.8 mmol), and Pd(dppf)Cl₂ (214 mg, 0.29 mmol) were dissolved in 1,4-dioxane (30 mL) and water (6 mL) was added, the reaction mixture was purged with nitrogen three times, then heated to 100° C. and reacted for 2 hours, and the starting materials were completely consumed as indicated by LC-MS. After cooling the reaction liquid to room temperature, water (40 mL) was added, and the mixture was extracted with ethyl acetate (40 mL×3). The organic layers were combined, washed with saturated brine (20 mL), dried over anhydrous sodium sulfate, filtered, and concentrated under vacuum. The residue was then purified by silica gel chromatography (eluent: petroleum ether/ethyl acetate=1:1) to afford tert-butyl (1-(4-(benzyloxy)-6-(4-cyano-3-fluorophenyl)-5-(3-hydroxy-4-methoxyphenyl)pyridin-2-yl)piperidin-4-yl)carbamate with a yield of 43.8%.

ESI-MS m/z=625.3 [M+H]⁺.

### Step e): preparation of tert-butyl (1-(6-(4-cyano-3-fluorophenyl)-4-hydroxy-5-(3-hydroxy-4-methoxyphenyl)pyridin-2-yl)piperidin-4-yl)carbamate

Tert-butyl (1-(4-(benzyloxy)-6-(4-cyano-3-fluorophenyl)-5-(3-hydroxy-4-methoxyphenyl)pyridin-2-yl)piperidin-4-yl)carbamate (200 mg, 0.32 mmol) was dissolved in methanol (20 mL), and 100 mg of 10% Pd(OH)₂ was added. The mixture was purged with hydrogen three times, then reacted at room temperature for 4 hours. The reaction liquid was ultrasonicated for 15 minutes and then filtered, and the filtrate was concentrated under vacuum to afford tert-butyl (1-(6-(4-cyano-3-fluorophenyl)-4-hydroxy-5-(3-hydroxy-4-methoxyphenyl)pyridin-2-yl)piperidin-4-yl)carbamate with a yield of 96.8%.

ESI-MS m/z=535.2 [M+H]⁺.

### Step f): preparation of methyl 7-((6-(4-((tert-butoxycarbonyl)amino)piperidin-1-yl)-2-(4-cyano-3-fluorophenyl)-3-(3-hydroxy-4-methoxyphenyl)pyridin-4-yl)oxy)heptanoate

Tert-butyl (1-(6-(4-cyano-3-fluorophenyl)-4-hydroxy-5-(3-hydroxy-4-methoxyphenyl)pyridin-2-yl)piperidin-4-yl)carbamate (150 mg, 0.22 mmol) was dissolved in acetonitrile (3 mL), followed by adding diisopropylethylamine (86 mg, 0.66 mmol) and methyl 7-bromoheptanoate (74 mg, 0.33 mmol). The solution was stirred at 60° C. overnight. The mixture was concentrated under vacuum, and the residue was purified by silica gel column (eluent: petroleum ether:ethyl acetate=1:1) to afford methyl 7-((6-(4-((tert-butoxycarbonyl)amino)piperidin-1-yl)-2-(4-cyano-3-fluorophenyl)-3-(3-hydroxy-4-methoxyphenyl)pyridin-4-yl)oxy)heptanoate with a yield of 87%.

ESI-MS m/z=677.3 [M+H]⁺.

### Step g): preparation of 7-((6-(4-((tert-butoxycarbonyl)amino)piperidin-1-yl)-2-(4-cyano-3-fluorophenyl)-3-(3-hydroxy-4-methoxyphenyl)pyridin-4-yl)oxy)heptanoic acid

Methyl 7-((6-(4-((tert-butoxycarbonyl)amino)piperidin-1-yl)-2-(4-cyano-3-fluorophenyl)-3-(3-hydroxy-4-methoxyphenyl)pyridin-4-yl)oxy)heptanoate (135 mg, 0.20 mmol) was dissolved in a mixture of THF/H₂O=5:1 (3 mL) and the reaction was conducted at room temperature overnight, the pH of thesolution was adjusted to 2˜3 with 2 N HCl, followed by extraction with ethyl acetate, and the organic phase was washed once with brine and then dried and concentrated, and the residue was used directly for the subsequent step.

ESI-MS m/z=663.3 [M+H]⁺.

### Step h): preparation of tert-butyl (1-(6-(4-cyano-3-fluorophenyl)-5-(3-hydroxy-4-methoxyphenyl)-4-((7-oxo-7-((tetrahydro-2H-pyran-2-yl)oxy)amino)heptyl)oxy)pyridin-2-yl)piperidin-4-yl)carbamate

7-((6-(4-((Tert-butoxycarbonyl)amino)piperidin-1-yl)-2-(4-cyano-3-fluorophenyl)-3-(3-hydroxy-4-methoxyphenyl)pyridin-4-yl)oxy)heptanoic acid (132 mg, 0.20 mmol) was dissolved in DMF (3 mL), and HATU (114.1 mg, 0.3 mmol) and O-(tetrahydro-2H-pyran-2-yl)hydroxylamine (47 mg, 0.4 mmol) were added, the reaction was conducted at room temperature for 1 hour, then quenched with water, and extracted with ethyl acetate, and the organic phase was washed twice with water, dried, and concentrated under vacuum. The residue was purified by silica gel column (eluent: petroleum ether:ethyl acetate=1:1) to afford tert-butyl (1-(6-(4-cyano-3-fluorophenyl)-5-(3-hydroxy-4-methoxyphenyl)-4-((7-oxo-7-((tetrahydro-2H-pyran-2-yl)oxy)amino)heptyl)oxy)pyridin-2-yl)piperidin-4-yl)carbamate with a yield of 85%.

ESI-MS m/z=762.4 [M+H]⁺.

### Step i): preparation of 7-((6-(4-aminopiperidin-1-yl)-2-(4-cyano-3-fluorophenyl)-3-(3-hydroxy-4-methoxyphenyl)pyridin-4-yl)oxy)-N-hydroxyheptanamide hydrochloride

Tert-butyl (1-(6-(4-cyano-3-fluorophenyl)-5-(3-hydroxy-4-methoxyphenyl)-4-((7-oxo-7-((tetrahydro-2H-pyran-2-yl)oxy)amino)heptyl)oxy)pyridin-2-yl)piperidin-4-yl)carbamate (130 mg, 0.17 mmol) was dissolved in a solution of HCl/EA (3 mL, 4M) and the reaction was conducted at room temperature for 1 hour. Solid was formed in the reaction liquid, and LC-MS analysis indicated that the reaction was completed. The crude product was collected by filtration, and was purified by Prep-HPLC (separation method 1) to afford 7-((6-(4-aminopiperidin-1-yl)-2-(4-cyano-3-fluorophenyl)-3-(3-hydroxy-4-methoxyphenyl)pyridin-4-yl)oxy)-N-hydroxyheptanamide hydrochloride with a yield of 65.2%.

¹H NMR (400 MHZ, DMSO-d₆) δ 10.41 (d, J=27.2 Hz, 1H), 8.39 (s, 3H), 7.86 (t, J=7.4 Hz, 1H), 7.50 (d, J=10.6 Hz, 1H), 7.29 (dd, J=8.2, 1.4 Hz, 1H), 6.84 (d, J=8.2 Hz, 1H), 6.70 (s, 1H), 6.59 (d, J=2.0 Hz, 1H), 6.45 (dd, J=8.2, 2.0 Hz, 1H), 4.47 (d, J=13.3 Hz, 2H), 4.17 (t, J=6.3 Hz, 2H), 3.79 (s, 3H), 3.39 (dq, J=11.5, 5.5 Hz, 1H), 3.13 (t, J=12.9 Hz, 2H), 2.13-2.04 (m, 2H), 1.98 (t, J=7.4 Hz, 2H), 1.68 (tq, J=13.6, 6.8, 5.2 Hz, 4H), 1.51 (p, J=7.4 Hz, 2H), 1.30 (dq, J=19.9, 6.6, 5.3 Hz, 4H).

ESI-MS m/z=578.3 [M+H]⁺.

The compounds of Examples 2-18 were prepared similarly according to the synthetic method of Example 1 (the separation method for the compound: hydrochloride, trifluoroacetate, formate, and free base were prepared by separation methods 1, 2, 3, and 4, respectively), and the structure and characterization data are as follows:

| Ex- | | | | MS |
|---|---|---|---|---|
| am- | | | | (M + |
| ple | Chemical name | Structure | ¹H NMR | H)⁺ |
| 2 | (E)-3-(4-(((6-(4- Aminopiperidin-1-yl)- 2-(4-cyano-3- fluorophenyl)-3-(3- hydroxy-4- methoxyphenyl) pyridin-4- yl)oxy)methyl)phenyl)- N-hydroxyacrylamide hydrochloride | 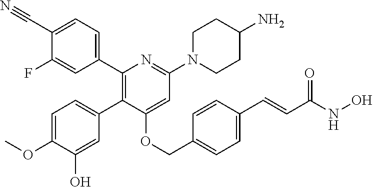 [SMILES: COc1ccc(-c2c(OCc3ccc(/C=C/C(=O)NO)cc3)cc(N3CCC(N)CC3)nc2-c2ccc(C#N)c(F)c2)cc1O] | ¹H NMR (400 MHz, DMSO-d₆) δ ppm: 10.82 (s, 1H), 8.17 (s, 3H), 7.77 (t, J = 7.6 Hz, 1H), 7.54 (d, J = 8.0 Hz, 2H), 7.43 (d, J = 15.8 Hz, 1H), 7.36 (d, J = 8.4 Hz, 3H), 7.21 (dt, J = 8.2, 1.4 Hz, 1H), 6.80 (d, J = 8.4 Hz, 1H), 6.69 (s, 1H), 6.62 (d, J = 2.0 Hz, 1H), 6.53-6.39 (m, 2H), 5.27 (s, 2H), 4.42 (d, J = 13.4 Hz, 2H), 3.74 (s, 3H), 3.38-3.23 (m, 1H), 2.98 (t, J = 12.8 Hz, 2H), 1.98 (d, J = 12.0 Hz, 2H), 1.65- 1.44 (m, 2H). | 610.3 |
| 3 | 7-(6-(4- Aminopiperidin-1-yl)- 2-(4-cyano-3- fluorophenyl)-3-(3- fluoro-4- methoxyphenyl) pyridin-4-yl)oxy-N- hydroxyheptanamide hydrochloride | 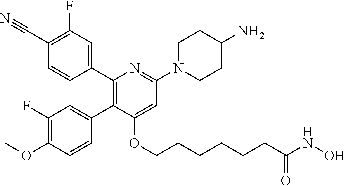 [SMILES: COc1ccc(-c2c(OCCCCCCC(=O)NO)cc(N3CCC(N)CC3)nc2-c2ccc(C#N)c(F)c2)cc1F] | ¹H NMR (400 MHz, DMSO-d₆) δ ppm: 10.34 (s, 1H), 9.02 (s, 1H), 8.12 (s, 3H), 7.77 (t, J = 7.6 Hz, 1H), 7.37 (d, J = 10.6 Hz, 1H), 7.16 (d, J = 8.2 Hz, 1H), 7.05- 6.94 (m, 2H), 6.71 (d, J = 8.4 Hz, 1H), 6.58 (s, 1H), 4.45 (d, J = 13.5 Hz, 2H), 4.06 (t, J = 6.3 Hz, 2H), 3.81 (s, 3H), 3.31 (m, 1H), 2.97 (t, J = 12.6 Hz, 2H), 1.98 (d, J = 12.4 Hz, 2H), 1.91 (t, J = 7.4 Hz, 2H), 1.56 (dq, J = 12.4, 6.0, 5.2 Hz, 4H), 1.43 (q, J = 7.2 Hz, 2H), 1.25 (dp, J = 21.0, 6.8 Hz, 4H). | 580.3 |
| 4 | 7-(6-(4- Aminopiperidin-1-yl)- 2-(4-cyano-3- fluorophenyl)-3-(4- (methylsulfonyl)phenyl) pyridin-4-yl)oxy)-N- hydroxyheptanamide hydrochloride | 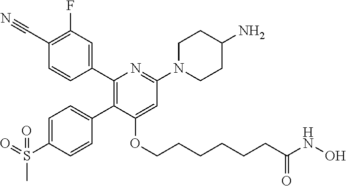 [SMILES: CS(=O)(=O)c1ccc(-c2c(OCCCCCCC(=O)NO)cc(N3CCC(N)CC3)nc2-c2ccc(C#N)c(F)c2)cc1] | ¹H NMR (400 MHz, DMSO-d₆) δ ppm: 10.37 (s, 1H), 8.27 (s, 3H), 7.76 (dd, J = 16.6, 7.8 Hz, 3H), 7.35 (m, 3H), 7.10 (d, J = 8.2 Hz, 1H), 6.63 (s, 1H), 4.48 (d, J = 13.4 Hz, 2H), 4.09 (t, J = 6.4 Hz, 2H), 3.32 (s, 1H), 3.21 (s, 3H), 3.00 (t, J = 12.6 Hz, 2H), 2.01 (d, J = 12.2 Hz, 2H), 1.92 (q, J = 9.4, 7.4 Hz, 2H), 1.57 (h, J = 9.2, 8.4 Hz, 4H), 1.42 (p, J = 7.4 Hz, 2H), 1.24 (d, J = 15.2 Hz, 4H). | 610.3 |
| 5 | 7-(3-(4-Aminophenyl)- 6-(4-aminopiperidin-1- yl)-2-(4-cyano-3- fluorophenyl)pyridin-4- yl)oxy)-N- hydroxyheptanamide hydrochloride | 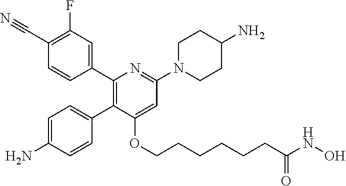 [SMILES: N#Cc1ccc(-c2nc(N3CCC(N)CC3)cc(OCCCCCCC(=O)NO)c2-c2ccc(N)cc2)cc1F] | ¹H NMR (400 MHz, DMSO-d₆) δ ppm: 10.23 (s, 2H), 8.28-8.05 (m, 2H), 7.75 (t, J = 7.6 Hz, 1H), 7.26 (m, 3H), 7.16 (dd, J = 10.8, 8.2 Hz, 3H), 6.59 (s, 1H), 4.46 (d, J = 13.4 Hz, 2H), 4.06 (t, J = 6.4 Hz, 2H), 3.30 (d, J = 15.4 Hz, 1H), 2.97 (t, J = 12.8 Hz, 2H), 1.98 (dd, J = 13.0, 4.0 Hz, 2H), 1.92 (t, J = 7.4 Hz, 2H), 1.55 (q, J = 6.0, 5.0 Hz, 4H), 1.43 (p, J = 7.4 Hz, 2H), 1.22 (dt, J = 18.4, 8.8 Hz, 4H). | 547.3 |
| 6 | 7-(6-(4- Aminopiperidin-1-yl)- 2-(4-cyano-3- fluorophenyl)-3-(3,4- difluorophenyl)pyridin- 4-yl)oxy)-N- hydroxyheptanamide hydrochloride | 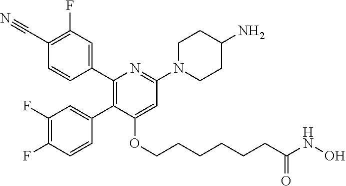 [SMILES: N#Cc1ccc(-c2nc(N3CCC(N)CC3)cc(OCCCCCCC(=O)NO)c2-c2ccc(F)c(F)c2)cc1F] | ¹H NMR (400 MHz, DMSO-d₆) δ ppm: 10.35 (s, 1H), 8.57-8.29 (m, 3H), 7.81 (t, J = 7.4 Hz, 1H), 7.47 (d, J = 10.4 Hz, 1H), 7.31 (dd, J = 10.8, 8.4 Hz, 1H), 7.28-7.22 (m, 1H), 7.19 (t, J = 6.4 Hz, 2H), 6.91-6.76 (m, 1H), 6.66 (s, 1H), 4.45 (d, J = 13.4 Hz, 2H), 4.12 (t, J = 6.2 Hz, 2H), 3.32 (tt, J = 10.6, 5.4 Hz, 1H), 3.06 (t, J = 12.6 Hz, 2H), 2.11-1.98 (m, 2H), 1.92 (t, J = 7.4 Hz, 2H), 1.61 (dp, J = 19.6, 6.4, 5.2 Hz, 4H), 1.43 (p, J = 7.4 Hz, 2H), 1.23 (t, J = 10.8 Hz, 4H). | 568.3 |
| 7 | 7-[(6-(4- Aminopiperidin-1-yl)- 2-(4-cyano-3- fluorophenyl)-3-(4- methylphenyl)pyridin- 4-yl)oxy]-N- hydroxyheptanamide- hydrochloride | 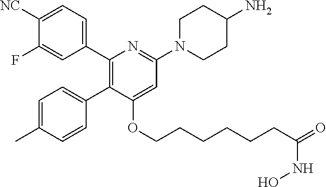 [SMILES: Cc1ccc(-c2c(OCCCCCCC(=O)NO)cc(N3CCC(N)CC3)nc2-c2ccc(C#N)c(F)c2)cc1] | ¹H NMR (400 MHz, Methanol-d₄) δ ppm 7.72 (d, J = 7.6 Hz, 1H), 7.49 (d, J = 9.8 Hz, 1H), 7.34 (d, J = 8.2 Hz, 1H), 7.13 (d, J = 7.6 Hz, 2H), 7.00 (d, J = 7.8 Hz, 2H), 6.81 (s, 1H), 4.37 (d, J = 13.8 Hz, 2H), 4.30 (d, J = 5.8 Hz, 2H), 3.56 (s, 1H), 3.42 (t, J = 13.2 Hz, 2H), 2.33 (s, 3H), 2.25 (d, J = 13.4 Hz, 2H), 2.04 (d, J = 7.6 Hz, 2H), 1.88 (q, J = 12.6 Hz, 2H), 1.72 (d, J = 8.2 Hz, 2H), 1.56 (t, J = 7.6 Hz, 2H), 1.32 (t, J = 11.2 Hz, 4H). | 546.3 |
| 8 | 7-[(6-(4- Aminopiperidin-1-yl)- 2-(4-cyano-3- fluorophenyl)-3-(4- methoxyphenyl) pyridin-4-yl)oxy]-N- hydroxyheptanamide trifluoroacetate | 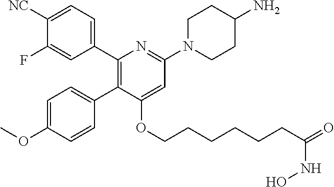 [SMILES: COc1ccc(-c2c(OCCCCCCC(=O)NO)cc(N3CCC(N)CC3)nc2-c2ccc(C#N)c(F)c2)cc1] | ¹H NMR (400 MHz, DMSO-d₆) δ ppm 7.57 (t, J = 7.4 Hz, 1H), 7.31 (d, J = 10.4 Hz, 1H), 7.23 (d, J = 8.1 Hz, 1H), 6.98 (d, J = 8.4 Hz, 2H), 6.84 (d, J = 8.3 Hz, 2H), 6.58 (s, 1H), 4.49 (d, J = 13.6 Hz, 2H), 4.11 (t, J = 6.1 Hz, 2H), 3.78 (s, 3H), 3.45 (ddt, J = 11.6, 7.9, 4.3 Hz, 1H), 3.14 (t, J = 12.8 Hz, 2H), 2.13 (dd, J = 12.8, 3.9 Hz, 2H), 2.04 (t, J = 7.4 Hz, 2H), 1.71 (d, J = 1 7.4, 6.4 Hz, 4H), 1.56 (p, J = 7.4 Hz, 2H), 1.43-1.20 (m, 4H). | 562.3 |
| 9 | 7-((6-(3-Amino-8- azabicyclo[3.2.1]octan- 8-yl)-2-(4-cyano-3- fluorophenyl)-3-(3- hydroxy-4- methoxyphenyl) pyridin-4-yl)oxy)-N- hydroxyheptanamide hydrochloride | 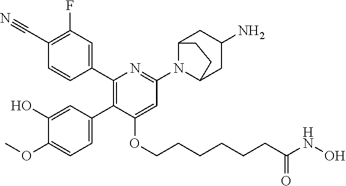 [SMILES: COc1ccc(-c2c(OCCCCCCC(=O)NO)cc(N3C4CCC3CC(N)C4)nc2-c2ccc(C#N)c(F)c2)cc1O] | ¹H NMR (400 MHz, Methanol-d₄) δ 7.72 (t, J = 7.2 Hz, 1H), 7.51 (d, J = 9.6 Hz, 1H), 7.35 (d, J = 8.0 Hz, 1H), 6.80 (d, J = 8.2 Hz, 1H), 6.70 (s, 1H), 6.65-6.53 (m, 1H), 6.53-6.38 (m, 1H), 4.92 (s, 2H), 4.27 (t, J = 6.0 Hz, 2H), 3.81 (s, 4H), 2.35-2.21 (m, 2H), 2.21- 2.10 (m, 2H), 2.10-1.96 (m, 6H), 1.85-1.66 (m, 2H), 1.55 (t, J = 7.4 Hz, 2H), 1.33 (dp, J = 12.6, 7.2 Hz, 4H). | 604.3 |
| 10 | 7-((6-(4- Aminopiperidin-1-yl)- 2-(4-cyano-3- fluorophenyl)-2′- methyl-[3,4′-bipyridin]- 4-yl)oxy)-N- hydroxyheptanamide hydrochloride | 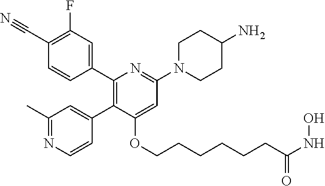 [SMILES: Cc1cc(-c2c(OCCCCCCC(=O)NO)cc(N3CCC(N)CC3)nc2-c2ccc(C#N)c(F)c2)ccn1] | ¹H NMR (400 MHz, Methanol-d₄) δ 8.12 (dd, J = 7.6, 6.5 Hz, 1H), 7.56 (m, 3H), 7.2 (d, J = 8.2 Hz, 2H), 6.9 (s, 1H), 4.65 (d, J = 12.6 Hz, 2H), 4.28 (t, J = 7.2 Hz, 2H), 3.56 (s, 1H), 3.41 (t, J = 12.9 Hz, 2H), 2.69(s, 3H), 2.31-2.17 (m, 2H), 2.05 (t, J = 7.3 Hz, 2H), 1.95- 1.79 (m, 2H), 1.78-1.64 (m, 2H), 1.55 (d, J = 7.2 Hz, 2H), 1.31 (dd, J = 10.9, 6.6 Hz, 4H). | 547.3 |
| 11 | 7-((6-(4- Aminopiperidin-1-yl)- 3-(4-chlorophenyl)-2- (4-cyano-3- fluorophenyl)pyridin-4- yl)oxy)-N- hydroxyheptanamide hydrochloride | 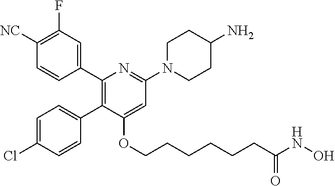 [SMILES: N#Cc1ccc(-c2nc(N3CCC(N)CC3)cc(OCCCCCCC(=O)NO)c2-c2ccc(Cl)cc2)cc1F] | ¹H NMR (400 MHz, Methanol-d₄) δ 7.73 (dd, J = 7.9, 6.5 Hz, 1H), 7.53 (d, J = 9.6 Hz, 1H), 7.32 (m, 3H), 7.11 (d, J = 8.2 Hz, 2H), 6.80 (s, 1H), 4.37 (d, J = 13.7 Hz, 2H), 4.28 (t, J = 6.1 Hz, 2H), 3.56 (d, J = 11.7 Hz, 1H), 3.41 (t, J = 12.9 Hz, 2H), 2.31-2.17 (m, 2H), 2.05 (t, J = 7.3 Hz, 2H), 1.95-1.79 (m, 2H), 1.78-1.64 (m, 2H), 1.55 (p, J = 7.2 Hz, 2H), 1.31 (qd, J = 10.9, 6.6 Hz, 4H). | 566.2 |
| 12 | 7-((6-(4- Aminopiperidin-1-yl)- 2-(4-cyanophenyl)-3- (3-hydroxy-4- methoxyphenyl) pyridin-4-yl)oxy)-N- hydroxyheptanamide (free) | 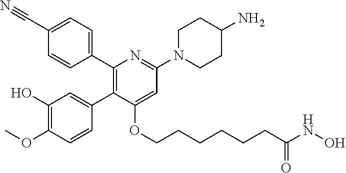 [SMILES: COc1ccc(-c2c(OCCCCCCC(=O)NO)cc(N3CCC(N)CC3)nc2-c2ccc(C#N)cc2)cc1O] | ¹H NMR (400 MHz, DMSO-d₆) δ 7.71 (d, J = 8.1 Hz, 2H), 7.45 (d, J = 8.1 Hz, 2H), 6.80 (d, J = 8.3 Hz, 1H), 6.55 (s, 1H), 6.49 (s, 1H), 6.41 (d, J = 8.0 Hz, 1H), 4.34 (d, J = 12.4 Hz, 2H), 4.07 (t, J = 6.4 Hz, 2H), 3.78 (s, 3H), 2.96 (t, J = 12.3 Hz, 2H), 2.86 (s, 1H), 1.98 (t, J = 7.4 Hz, 2H), 1.83 (d, J = 12.2 Hz, 2H), 1.63 (p, J = 6.6 Hz, 2H), 1.51 (p, J = 7.4 Hz, 2H), 1.30 (dq, J = 14.2, 9.0, 8.1 Hz, 6H). | 560.3 |
| 13 | 7-((6-(4- Aminopiperidin-1-yl)- 2-(4-cyano-3- fluorophenyl)-3-(2- (dimethylamino) pyrimidin-5-yl)pyridin- 4-yl)oxy)-N- hydroxyheptanamide formate | 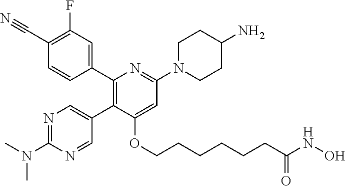 [SMILES: CN(C)c1ncc(-c2c(OCCCCCCC(=O)NO)cc(N3CCC(N)CC3)nc2-c2ccc(C#N)c(F)c2)cn1] | ¹H NMR (400 MHz, DMSO-d₆) δ ppm: 8.40 (s, 1H), 7.98 (s, 2H), 7.81 (t, J = 7.6 Hz, 1H), 7.43 (d, J = 10.6 Hz, 1H), 7.21 (d, J = 8.2 Hz, 1H), 6.54 (s, 1H), 4.39 (d, J = 13.2 Hz, 2H), 4.05 (t, J = 6.4 Hz, 2H), 3.12 (s, 2H), 3.08 (s, 6H), 2.93 (t, J = 12.6 Hz, 2H), 1.90 (q, J = 6.8 Hz, 3H), 1.61 (p, J = 6.6 Hz, 2H), 1.43 (tt, J = 12.2, 5.6 Hz, 4H), 1.25 (dd, J = 20.0, 7.2 Hz, 4H). | 577.3 |
| 14 | 7-((6-(4- Aminopiperidin-1-yl)- 2-(4-cyano-3- fluorophenyl)-3-(2- methyl-2H-indazol-5- yl)pyridin-4-yl)oxy)-N- hydroxyheptanamide formate | 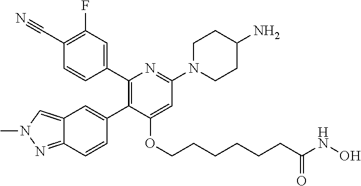 [SMILES: Cn1cc2cc(-c3c(OCCCCCCC(=O)NO)cc(N4CCC(N)CC4)nc3-c3ccc(C#N)c(F)c3)ccc2n1] | ¹H NMR (400 MHz, DMSO-d₆) δ ppm: 8.40 (d, J = 10.2 Hz, 1H), 8.17 (s, 1H), 7.70-7.61 (m, 1H), 7.47 (d, J = 8.8 Hz, 1H), 7.32 (dd, J = 10.8, 1.4 Hz, 1H), 7.25 (s, 1H), 7.15 (dd, J = 8.2, 1.4 Hz, 1H), 6.96 (dd, J = 8.8, 1.6 Hz, 1H), 6.54 (s, 1H), 4.39 (d, J = 13.4 Hz, 2H), 4.12 (s, 3H), 4.03 (t, J = 6.2 Hz, 2H), 3.57 (s, 3H), 2.93 (t, J = 12.0 Hz, 2H), 2.17 (t, J = 7.4 Hz, 2H), 1.89 (s, 2H), 1.53 (p, J = 6.4 Hz, 2H), 1.40 (p, J = 7.4 Hz, 4H), 1.20 (dq, J = 22.2, 8.2 Hz, 4H). | 586.3 |
| 15 | 7-((6-(5-Amino-2- azabicyclo[2.2.1]heptan- 2-yl)-2-(4-cyano-3- fluorophenyl)-3-(3- hydroxy-4- methoxyphenyl)pyridin- 4-yl)oxy)-N- hydroxyheptanamide hydrochloride | 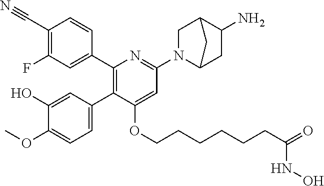 [SMILES: COc1ccc(-c2c(OCCCCCCC(=O)NO)cc(N3CC4CC3CC4N)nc2-c2ccc(C#N)c(F)c2)cc1O] | ¹H NMR (400 MHz, Methanol-d₄) δ 7.71 (ddd, J = 8.1, 6.6, 3.0 Hz, 1H), 7.63-7.41 (m, 1H), 7.41- 7.18 (m, 1H), 6.79 (d, J = 8.2 Hz, 1H), 6.58 (s, 1H), 6.44 (s, 2H), 4.78 (s, 1H), 4.24 (dp, J = 13.8, 4.4 Hz, 2H), 3.90 (dq, J = 14.6, 5.2, 4.7 Hz, 2H), 3.81 (s, 4H), 3.13 (td, J = 3.6, 1.8 Hz, 1H), 2.53-2.31 (m, 1H), 2.15 (dd, J = 11.2, 11.0 Hz, 1H), 2.03 (t, J = 7.4 Hz, 3H), 1.94- 1.79 (m, 2H), 1.72 (p, J = 6.4 Hz, 2H), 1.55 (p, J = 7.4 Hz, 2H), 1.31 (dq, J = 10.6, 6.8, 6.0 Hz, 4H). | 590.3 |
| 16 | 7-((6-(4- Aminopiperidin-1-yl)- 2-(4-cyano-3- methylphenyl)-3-(3- hydroxy-4- methoxyphenyl)pyridin- 4-yl)oxy)-N- hydroxyheptanamide hydrochloride | 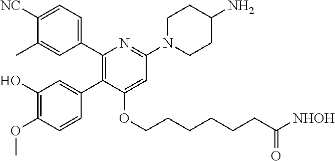 | ¹H NMR (400 MHz, Methanol-d₄) δ ppm 7.61 (d, J = 8.0 Hz, 1H), 7.48 (s, 1H), 7.28 (d, J = 8.2 Hz, 1H), 6.83-6.73 (m, 2H), 6.59 (d, J = 2.0 Hz, 1H), 6.45 (dd, J = 8.2, 2.2 Hz, 1H), 4.37-4.23 (m, 4H), 3.82 (s, 3H), 3.54 (dt, J = 11.6, 7.0 Hz, 1H), 3.39 (t, J = 12.8 Hz, 2H), 2.49 (s, 3H), 2.27-2.19 (m, 2H), 2.05 (t, J = 7.4 Hz, 2H), 1.93-1.79 (m, 2H), 1.74 (t, J = 6.6 Hz, 2H), 1.56 (p, J = 7.4 Hz, 2H), 1.34 (dt, J = 22.6, 7.6 Hz, 4H). | 574.3 |
| 17 | 7-((6-(4- Aminopiperidin-1-yl)- 2-(4-cyano-3- fluorophenyl)-3-(4-(2- hydroxyethoxy)phenyl) pyridin-4-yl)oxy)-N- hydroxyheptanamide formate | 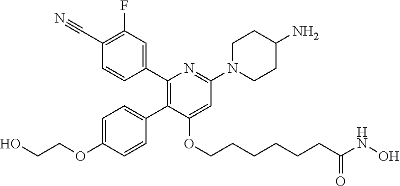 [SMILES: N#Cc1ccc(-c2nc(N3CCC(N)CC3)cc(OCCCCCCC(=O)NO)c2-c2ccc(OCCO)cc2)cc1F] | ¹H NMR (400 MHz, Methanol-d₄) δ 8.55 (s, 1H), 7.49 (t, J = 7.4 Hz, 1H), 7.29-7.16 (m, 2H), 6.96 (d, J = 8.6 Hz, 2H), 6.86 (d, J = 8.4 Hz, 2H), 6.46 (s, 1H), 4.52 (d, J = 13.4 Hz, 2H), 4.12-3.97 (m, 4H), 3.87 (t, J = 4.7 Hz, 2H), 3.00 (t, J = 12.2 Hz, 2H), 2.03 (q, J = 8.2, 7.7 Hz, 4H), 1.77-1.49 (m, 6H), 1.31 (dp, J = 20.6, 7.5 Hz, 5H). | 592.3 |
| 18 | 7-((6-(4- Aminopiperidin-1-yl)- 3-(3-hydroxy-4- methoxyphenyl)-2′- methyl-[2,4′-bipyridin]- 4-yl)oxy)-N- hydroxyheptanamide diformate | 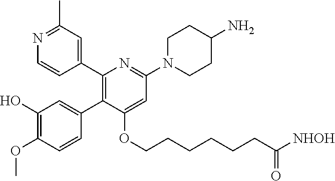 | ¹H NMR (400 MHz, Methanol-d₄) δ ppm 8.55 (s, 2H), 8.16 (d, J = 5.4 Hz, 1H), 7.20 (s, 1H), 7.07 (dd, J = 5.4, 1.8 Hz, 1H), 6.79 (d, J = 8.2 Hz, 1H), 6.58 (d, J = 2.2 Hz, 1H), 6.49-6.37 (m, 2H), 4.53 (d, J = 13.6 Hz, 2H), 4.03 (t, J = 6.2 Hz, 2H), 3.83 (s, 3H), 3.00 (t, J = 12.6 Hz, 2H), 2.39 (s, 3H), 2.04 (q, J = 7.8 Hz, 4H), 1.61 (m, 6H), 1.32 (dq, J = 23.6, 7.4 Hz, 4H). | 550.3 |

### Example 19

### Preparation of 7-(4-(6-(4-aminopiperidin-1-yl)-2-(4-cyano-3-fluorophenyl)-4-hydroxypyridin-3-yl)-2-hydroxyphenoxy)-N-hydroxyheptanamide hydrochloride

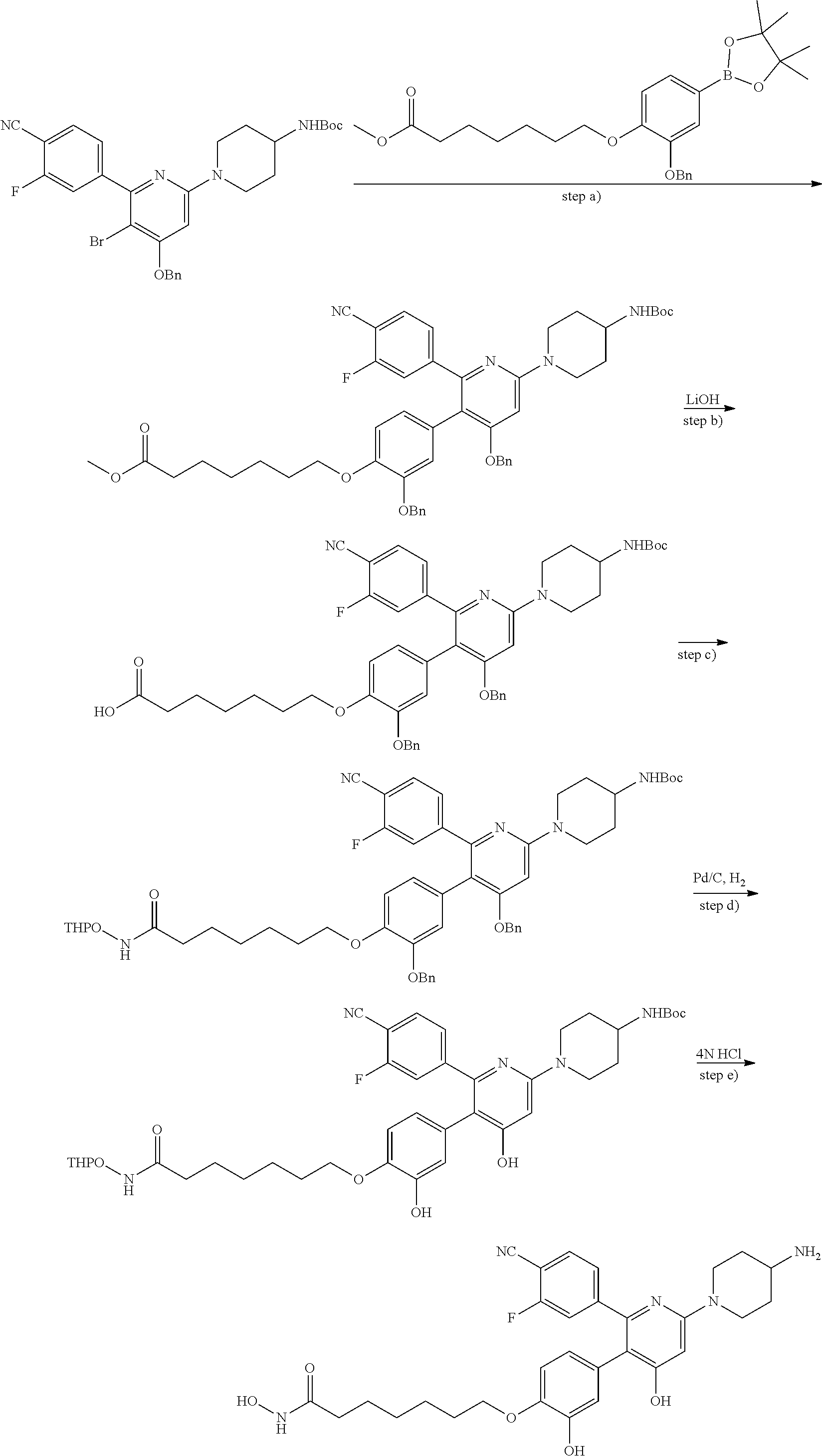

### Step a): preparation of methyl 7-(2-(benzyloxy)-4-(4-(benzyloxy)-6-(4-(tert-butoxycarbonyl)amino)piperidin-1-yl)-2-(4-cyano-3-fluorophenyl)pyridin-3-yl)phenoxy)heptanoate

The product of Example 1 Step c): tert-butyl (1-(4-(benzyloxy)-5-bromo-6-(4-cyano-3-fluorophenyl)pyridin-2-yl)piperidin-4-yl)carbamate (200 mg, 345 μmol), methyl 7-(2-(benzyloxy)-4-(4,4,5,5-tetramethyl-1,3,2-dioxaborolan-2-yl)phenoxy)heptanoate (241 mg, 517 μmol), Cs₂CO₃ (225 mg, 690 μmol), Pd(dppf)Cl₂ (26 mg, 35 μmol), 1,4-dioxane (10 mL), and H₂O (2.5 mL) were added in a reaction flask, and the reaction was stirred at 120° C. for 1 hour. The mixture was concentrated to dryness under reduced pressure, and the residue was then purified by silica gel chromatography (eluent: petroleum ether/ethyl acetate=1/2) to afford methyl 7-(2-(benzyloxy)-4-(4-(benzyloxy)-6-(4-(tert-butoxycarbonyl)amino)piperidin-1-yl)-2-(4-cyano-3-fluorophenyl)pyridin-3-yl)phenoxy)heptanoate with a yield of 58.0%.

ESI-MS (m/z)=843.4 [M+H]⁺.

### Step b): preparation of 7-(2-(benzyloxy)-4-(4-(benzyloxy)-6-(4-(tert-butoxycarbonyl)amino)piperidin-1-yl)-2-(4-cyano-3-fluorophenyl)pyridin-3-yl)phenoxy)heptanoic acid

Methyl 7-(2-(benzyloxy)-4-(4-(benzyloxy)-6-(4-(tert-butoxycarbonyl)amino)piperidin-1-yl)-2-(4-cyano-3-fluorophenyl)pyridin-3-yl)phenoxy)heptanoate (169 mg, 200 μmol), lithium hydroxide monohydrate (42 mg, 1.0 mmol), tetrahydrofuran (2 mL), isopropanol (2 mL), and water (1 mL) were added to a reaction flask and stirred at room temperature for 12 hours. Under stirring in an ice bath, 1N concentrated hydrochloric acid was added dropwise to adjust the pH to 3˜4, and water (10 mL) was added, followed by extraction with ethyl acetate (10 mL×3), and the organic phases were combined, washed with saturated brine (10 mL×2), concentrated to dryness under reduced pressure to afford 7-(2-(benzyloxy)-4-(4-(benzyloxy)-6-(4-(tert-butoxycarbonyl)amino)piperidin-1-yl)-2-(4-cyano-3-fluorophenyl)pyridin-3-yl)phenoxy)heptanoic acid which was used directly for the next step. ESI-MS (m/z)=829.4 [M+H]⁺.

### Step c): preparation of tert-butyl (1-(4-(benzyloxy)-5-(3-(benzyloxy)-4-((7-oxo-7-((tetrahydro-2H-pyran-2-yl)oxy)amino)heptyl)oxy)phenyl)-6-(4-cyano-3-fluorophenyl)pyridin-2-yl)piperidin-4-yl)carbamate

7-(2-(Benzyloxy)-4-(4-(benzyloxy)-6-(4-(tert-butoxycarbonyl)amino)piperidin-1-yl)-2-(4-cyano-3-fluorophenyl)pyridin-3-yl)phenoxy)heptanoic acid (99 mg, 200 μmol), O-(tetrahydro-2H-pyran-2-yl)hydroxylamine (47 mg, 400 μmol), DIEA (52 mg, 400 μmol), and DMF (2 mL) were added to a reaction flask, and HATU (114 mg, 300 μmol) was added under stirring at room temperature, and the reaction was maintained for 1 hour at room temperature. After the reaction was completed, water (10 mL) was added to quench the reaction, followed by extraction with ethyl acetate (20 mL×2), and the organic phases were combined, washed sequentially with saturated sodium bicarbonate aqueous solution (20 mL×1) and saturated brine (10 mL×2), dried over anhydrous sodium sulfate, filtered, and the filtrate was concentrated under reduced pressure, and the residue was purified by silica gel chromatography (eluent: petroleum ether/ethyl acetate=1/1) to afford tert-butyl (1-(4-(benzyloxy)-5-(3-(benzyloxy)-4-((7-oxo-7-((tetrahydro-2H-pyran-2-yl)oxy)amino)heptyl)oxy)phenyl)-6-(4-cyano-3-fluorophenyl)pyridin-2-yl)piperidin-4-yl)carbamate with a yield of 62.7%.

ESI-MS (m/z)=928.5 [M+H]⁺.

### Step d): preparation of tert-butyl (1-(6-(4-cyano-3-fluorophenyl)-4-hydroxy-5-(3-hydroxy-4-((7-oxo-7-((tetrahydro-2H-pyran-2-yl)oxy)amino)heptyl)oxy)phenyl)pyridin-2-yl)piperidin-4-yl)carbamate

Tert-butyl (1-(4-(benzyloxy)-5-(3-(benzyloxy)-4-((7-oxo-7-((tetrahydro-2H-pyran-2-yl)oxy)amino)heptyl)oxy)phenyl)-6-(4-cyano-3-fluorophenyl)pyridin-2-yl)piperidin-4-yl)carbamate (117 mg, 126 μmol), palladium on carbon (25 mg, 5%), and ethanol (5 mL) were sequentially added to a reaction flask, stirred until dissolved, and the mixture was purged with hydrogen three times, and stirred at room temperature for 2 hours under hydrogen atmosphere. After the reaction was completed, it was filtered, and the filtrate was concentrated under reduced pressure to afford tert-butyl (1-(6-(4-cyano-3-fluorophenyl)-4-hydroxy-5-(3-hydroxy-4-((7-oxo-7-((tetrahydro-2H-pyran-2-yl)oxy)amino)heptyl)oxy)phenyl)pyridin-2-yl)piperidin-4-yl)carbamate with a yield of 85.0%.

ESI-MS (m/z)=748.4 [M+H]⁺.

### Step e): preparation of 7-(4-(6-(4-aminopiperidin-1-yl)-2-(4-cyano-3-fluorophenyl)-4-hydroxypyridin-3-yl)-2-hydroxyphenoxy)-N-hydroxyheptanamide hydrochloride

Tert-butyl (1-(6-(4-cyano-3-fluorophenyl)-4-hydroxy-5-(3-hydroxy-4-((7-oxo-7-((tetrahydro-2H-pyran-2-yl)oxy)amino)heptyl)oxy)phenyl)pyridin-2-yl)piperidin-4-yl)carbamate (80 mg, 107 μmol) was added to a reaction flask, followed by adding hydrogen chloride solution in ethyl acetate (4M, 2.5 mL), and the mixture was stirred at room temperature for 1 hour, resulting in the precipitation of a significant amount of solid. After concentrating under reduced pressure, the crude product was purified by Prep-HPLC (separation method 1) to afford 7-(4-(6-(4-aminopiperidin-1-yl)-2-(4-cyano-3-fluorophenyl)-4-hydroxypyridin-3-yl)-2-hydroxyphenoxy)-N-hydroxyheptanamide hydrochloride with a yield of 35.8%.

¹H NMR (400 MHZ, Methanol-d₄) δ 7.63 (t, J=7.2 Hz, 1H), 7.39 (d, J=9.6 Hz, 1H), 7.23 (d, J=7.8 Hz, 1H), 6.70 (d, J=8.2 Hz, 1H), 6.53 (d, J=6.2 Hz, 2H), 6.39 (d, J=8.0 Hz, 1H), 4.08 (d, J=13.2 Hz, 2H), 3.89 (t, J=6.4 Hz, 2H), 3.43 (td, J=11.6, 11.2, 5.4 Hz, 1H), 3.27 (d, J=12.6 Hz, 2H), 2.21-2.08 (m, 2H), 2.03 (t, J=7.4 Hz, 2H), 1.72 (dp, J=21.6, 7.6, 6.8 Hz, 4H), 1.62-1.48 (m, 2H), 1.38-1.28 (m, 4H).

ESI-MS (m/z)=564.3 [M+H]⁺.

### Example 20

### Preparation of 7-(5-(5-(4-aminopiperidin-1-yl)-7-(4-cyano-3-fluorophenyl)imidazo[1,2-c]pyrimidin-8-yl)-2-methoxyphenoxy)-N-hydroxyheptanamide hydrochloride

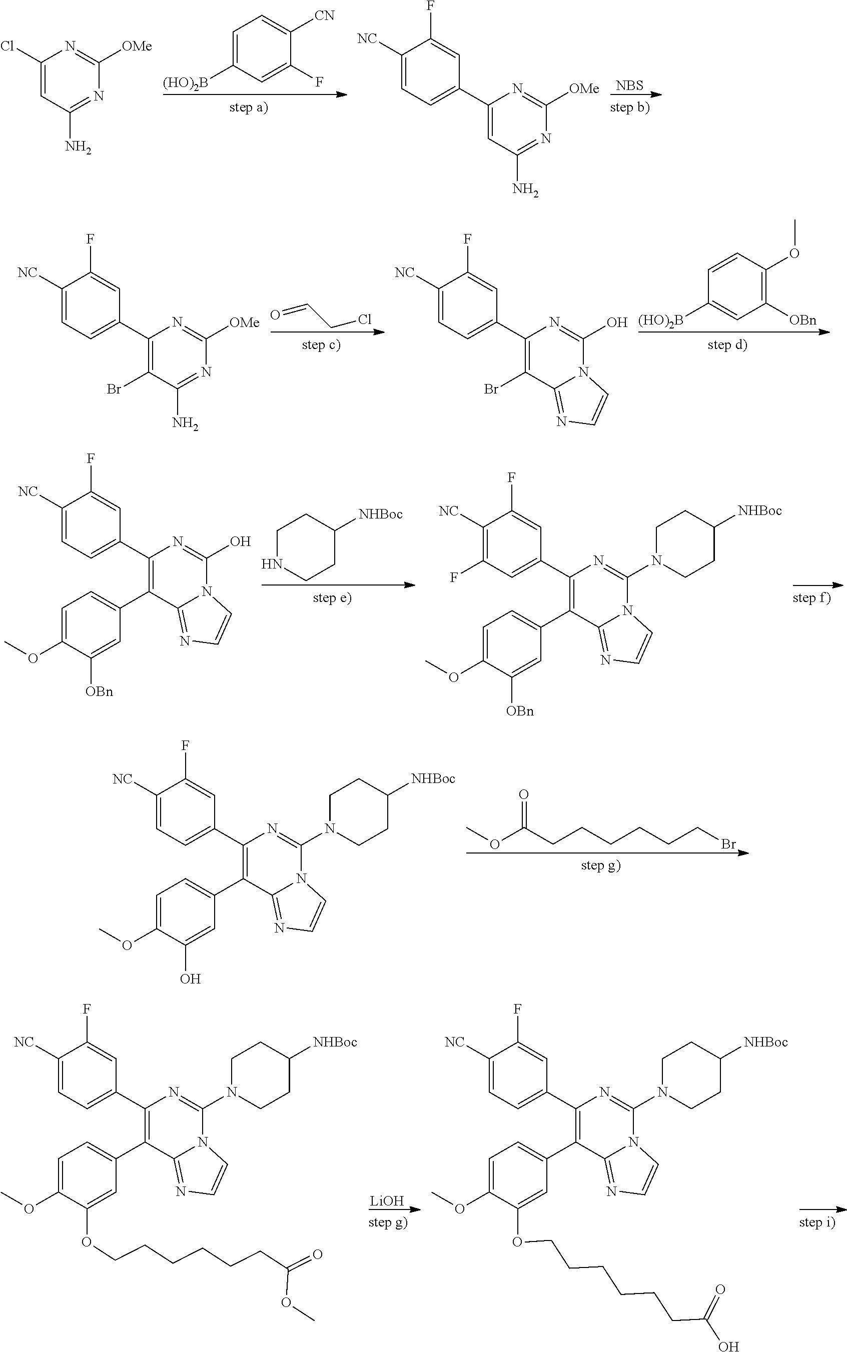

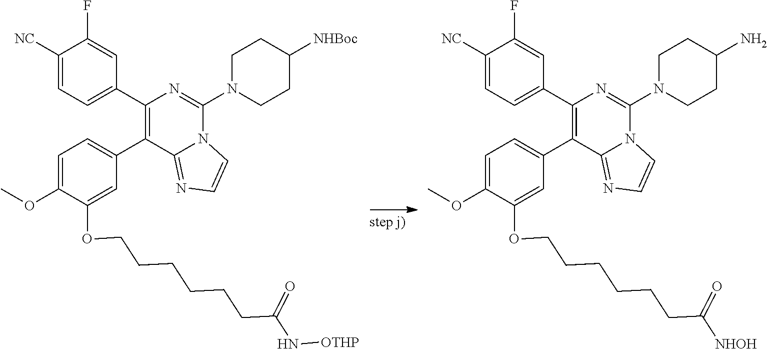

### Step a): preparation of 4-(6-amino-2-methoxypyrimidin-4-yl)-2-fluorobenzonitrile

6-Chloro-2-methoxypyrimidin-4-amine (3.0 g, 0.03 mol), Na₂CO₃ (9.9 g, 0.09 mol), (4-cyano-3-fluorophenyl)boronic acid (7.8 g, 0.05 mol), Pd(aphos)₂Cl₂ (4.3 g, 6 mmol) were dissolved in a mixture of 1,4-dioxane:water=5:1 (150 mL), and nitrogen was bubbled through the mixture The reaction was heated in an oil bath to 95° C. under nitrogen. After the reaction was completed as indicated by LCMS, the reaction liquid was concentrated under vacuum, then dissolved in ethyl acetate (20 mL), washed with water (20 mL), and the aqueous phase was extracted with ethyl acetate (20 mL×3), and the organic phases were washed with saturated brine, dried over anhydrous sodium sulfate, filtered, and concentrated, and the residue was purified by silica gel chromatography (eluent: petroleum ether/ethyl acetate=2/1) to afford 4-(6-amino-2-methoxypyrimidin-4-yl)-2-fluorobenzonitrile with a yield of 88.6%.

ESI-MS m/z: 245.1 [M+H]⁺.

### Step b): preparation of 4-(6-amino-5-bromo-2-methoxypyrimidin-4-yl)-2-fluorobenzonitrile

4-(6-Amino-2-methoxypyrimidin-4-yl)-2-fluorobenzonitrile (2.0 g, 8 mmol) was dissolved in dry DMSO (10 mL) and anhydrous acetonitrile (50 mL). Under nitrogen protection and cooling in an ice bath, NBS (1.45 g, 8 mmol) was added, and the reaction was continued for 2 hours. After the reaction was completed as indicated by LCMS, water (40 mL) was added to quench the reaction, the resultant mixture was then extracted with ethyl acetate (50 mL×3). The organic phases were combined and washed with saturated brine, dried with anhydrous sodium sulfate, filtered, and concentrated. The residue was purified by silica gel chromatography (eluent: petroleum ether/ethyl acetate=3/1) to afford 4-(6-amino-5-bromo-2-methoxypyrimidin-4-yl)-2-fluorobenzonitrile in a yield of 78.5%. ESI-MS m/z: 323.0 [M+H]⁺.

### Step c): preparation of 4-(8-bromo-5-hydroxyimidazopyrimidin-7-yl)-2-fluorobenzonitrile

4-(6-Amino-5-bromo-2-methoxypyrimidin-4-yl)-2-fluorobenzonitrile (500 mg, 1.55 mmol) was dissolved in isopropanol (13 mL), followed by adding chloroacetaldehyde (6.09 g, 31.06 mmol), the reaction liquid was purged with nitrogen and then heated at 110° C. overnight. After the reaction was completed as indicated by LCMS, the reaction liquid was concentrated under reduced pressure, and the resultant residue was purified by column chromatography on silica gel (eluent: methanol/dichloromethane=1/20) to afford 4-(8-bromo-5-hydroxyimidazopyrimidin-7-yl)-2-fluorobenzonitrile with a yield of 85.7%.

¹H NMR (400 MHZ, DMSO-d₆) δ ppm 12.28 (s, 1H), 8.14 (dt, J=9.0, 4.6 Hz, 1H), 7.97 (d, J=2.6 Hz, 1H), 7.84 (dd, J=10.2, 2.6 Hz, 1H), 7.65 (dd, J=7.8, 2.4 Hz, 1H), 7.50 (d, J=2.4 Hz, 1H). ESI-MS m/z: 333.0 [M+H]⁺.

### Step d): preparation of 4-(8-(3-(benzyloxy)-4-methoxyphenyl)-5-hydroxyimidazo[1,2-c]pyrimidin-7-yl)-2-fluorobenzonitrile

4-(8-Bromo-5-hydroxyimidazopyrimidin-7-yl)-2-fluorobenzonitrile (380 mg, 1.14 mmol), (3-(benzyloxy)-4-methoxyphenyl)boronic acid (292 mg, 1.72 mmol), Pd(dppf)Cl₂ (167 mg, 0.21 mmol), and Na₂CO₃ (243 mg, 2.29 mmol) were dissolved in 1,4-dioxane (15 mL) and water (3 mL) was added. The mixture was bubbled with N₂ and protected with N₂, and reacted in a microwave reactor at 105° C. for 30 minutes. After the reaction was completed as indicated by LCMS, the mixture was extracted with ethyl acetate (10 mL×3), washed with 10 mL saturated brine, dried with anhydrous sodium sulfate, filtered, and concentrated. The residue was purified by silica gel chromatography (eluent: methanol/dichloromethane=1/20) to afford 4-(8-(3-(benzyloxy)-4-methoxyphenyl)-5-hydroxyimidazo[1,2-c]pyrimidin-7-yl)-2-fluorobenzonitrile with a yield of 89.7%.

ESI-MS m/z: 467.1 [M+H]⁺.

### Step e): preparation of tert-butyl (1-(8-(3-(benzyloxy)-4-methoxyphenyl)-7-(4-cyano-3-fluorophenyl)imidazo[1,2-c]pyrimidin-5-yl)piperidin-4-yl)carbamate

4-(8-(3-(Benzyloxy)-4-methoxyphenyl)-5-hydroxyimidazo[1,2-c]pyrimidin-7-yl)-2-fluorobenzonitrile (200 mg, 0.530 mmol), tert-butylpiperidine-4-carbamate (318 mg, 1.57 mmol), and BOP Reagent (352 mg, 0.795 mmol) were dissolved in 16 mL anhydrous acetonitrile, then DIPEA (206 mg, 1.59 mmol) was added, the reaction liquid was bubbled with nitrogen and heated to 60° C. and reacted overnight under nitrogen. The progress of the reaction was monitored by TLC and LCMS. After the reaction was completed, the the reaction mixture was cooled and concentrated under reduced pressure, and the resultant residue was purified by thin-layer chromatography on a silica gel plate (eluent: methanol/dichloromethane=1/10) to afford tert-butyl (1-(8-(3-(benzyloxy)-4-methoxyphenyl)-7-(4-cyano-3-fluorophenyl)imidazo[1,2-c]pyrimidin-5-yl)piperidin-4-yl)carbamate with a yield of 58.4%.

ESI-MS m/z: 649.3 [M+H]⁺.

### Step f): preparation of tert-butyl (1-(7-(4-cyano-3-fluorophenyl)-8-(3-hydroxy-4-methoxyphenyl)imidazo[1,2-c]pyrimidin-5-yl)piperidin-4-yl)carbamate

Tert-butyl (1-(8-(3-(benzyloxy)-4-methoxyphenyl)-7-(4-cyano-3-fluorophenyl)imidazo[1,2-c]pyrimidin-5-yl)piperidin-4-yl)carbamate (200 mg, 309 μmol), palladium on carbon (50 mg, 5%), and ethanol (10 mL) were sequentially added to a reaction flask, the mixture was purged with hydrogen three times and stirred at room temperature for 2 hours under nitrogen protection. After the reaction was completed, the mixture was filtered, and the filtrate was concentrated under reduced pressure to afford tert-butyl (1-(7-(4-cyano-3-fluorophenyl)-8-(3-hydroxy-4-methoxyphenyl)imidazo[1,2-c]pyrimidin-5-yl)piperidin-4-yl)carbamate with a yield of 83.0%.

ESI-MS (m/z)=559.2 [M+H]⁺.

### Step g): preparation of methyl 7-(5-(5-(4-(tert-butoxycarbonyl)amino)piperidin-1-yl)-7-(4-cyano-3-fluorophenyl)imidazo[1,2-c]pyrimidin-8-yl)-2-methoxyphenoxy)heptanoate

Tert-butyl (1-(7-(4-cyano-3-fluorophenyl)-8-(3-hydroxy-4-methoxyphenyl)imidazo[1,2-c]pyrimidin-5-yl)piperidin-4-yl)carbamate (143 mg, 256 μmol), methyl 7-bromoheptanoate (114 mg, 512 μmol), K₂CO₃ (72 mg, 512 μmol), and acetonitrile (5 mL) were added to a reaction flask. The mixture was stirred for reaction at 75° C. for 12 hours and concentrated under reduced pressure, and the residue was then purified by silica gel chromatography (eluent: petroleum ether/ethyl acetate=8/3) to afford methyl 7-(5-(5-(4-(tert-butoxycarbonyl)amino)piperidin-1-yl)-7-(4-cyano-3-fluorophenyl)imidazo[1,2-c]pyrimidin-8-yl)-2-methoxyphenoxy)heptanoate with a yield of 57.0%. ESI-MS (m/z)=701.3 [M+H]⁺.

### Step h): preparation of 7-(5-(5-(4-((tert-butoxycarbonyl)amino)piperidin-1-yl)-7-(4-cyano-3-fluorophenyl)imidazo[1,2-c]pyrimidin-8-yl)-2-methoxyphenoxy)heptanoic acid

Methyl 7-(5-(5-(4-(tert-butoxycarbonyl)amino)piperidin-1-yl)-7-(4-cyano-3-fluorophenyl)imidazo[1,2-c]pyrimidin-8-yl)-2-methoxyphenoxy)heptanoate (102 mg, 146 μmol), lithium hydroxide monohydrate (31 mg, 730 μmol), THF (2 mL), isopropanol (2 mL), and water (2 mL) were added to a reaction flask. The mixture was stirred at room temperature for 8 hours. After the reaction was completed, 0.5N HCl aqueous solution (10 mL) was added, and the mixture was extracted with ethyl acetate (10 mL×3), and the organic phases were combined and washed with saturated brine (10 mL×2), concentrated under reduced pressure. The residue was purified by silica gel chromatography (eluent: petroleum ether/ethyl=acetate 8/3) to afford 7-(5-(5-(4-((tert-butoxycarbonyl)amino)piperidin-1-yl)-7-(4-cyano-3-fluorophenyl)imidazo[1,2-c]pyrimidin-8-yl)-2-methoxyphenoxy)heptanoic acid with a yield of 89.0%.

ESI-MS (m/z)=687.3 [M+H]⁺.

### Step i): preparation of tert-butyl (1-(7-(4-cyano-3-fluorophenyl)-8-(4-methoxy-3-((7-oxo-7-((tetrahydro-2H-pyran-2-yl)oxy)amino)heptyl)oxy)phenyl)imidazo[1,2-c]pyrimidin-5-yl)piperidin-4-yl)carbamate

7-(5-(5-(4-((Tert-butoxycarbonyl)amino)piperidin-1-yl)-7-(4-cyano-3-fluorophenyl)imidazo[1,2-c]pyrimidin-8-yl)-2-methoxyphenoxy)heptanoic acid (89 mg, 139 μmol), O-(tetrahydro-2H-pyran-2-yl)hydroxylamine (33 mg, 278 μmol), DIEA (36 mg, 278 μmol), and DMF (2 mL) were added to a reaction flask, and HATU (79 mg, 208 μmol) was added under stirring at room temperature. The reaction was stirred at room temperature for 1 hour. After the reaction was completed, water (10 mL) was added to quench the reaction, followed by extraction with ethyl acetate (20 mL×2), and the organic phases were combined and then washed sequentially with saturated sodium bicarbonate aqueous solution (20 mL×2) and saturated brine (10 mL×2). The organic phases were dried with anhydrous sodium sulfate, filtered and concentrated under reduced pressure, and the residue was purified by silica gel chromatography (eluent: petroleum ether/ethyl acetate=1/1) to afford tert-butyl (1-(7-(4-cyano-3-fluorophenyl)-8-(4-methoxy-3-((7-oxo-7-((tetrahydro-2H-pyran-2-yl)oxy)amino)heptyl)oxy)phenyl)imidazo[1,2-c]pyrimidin-5-yl)piperidin-4-yl)carbamate with a yield of 72.7%.

ESI-MS (m/z)=786.4 [M+H]⁺.

### Step j): preparation of 7-(5-(5-(4-aminopiperidin-1-yl)-7-(4-cyano-3-fluorophenyl)imidazo[1,2-c]pyrimidin-8-yl)-2-methoxyphenoxy)-N-hydroxyheptanamide hydrochloride

Tert-butyl (1-(7-(4-cyano-3-fluorophenyl)-8-(4-methoxy-3-((7-oxo-7-((tetrahydro-2H-pyran-2-yl)oxy)amino)heptyl)oxy)phenyl)imidazo[1,2-c]pyrimidin-5-yl)piperidin-4-yl)carbamate (79 mg, 101 μmol) was added to a reaction flask, followed by adding hydrogen chloride solution in ethyl acetate (4M, 2.5 mL), and the mixture was stirred at room temperature for 1 hour, resulting in the precipitation of a significant amount of solid. After concentrating under reduced pressure, the crude product was purified by Prep-HPLC (separation method 1) to afford 7-(5-(5-(4-aminopiperidin-1-yl)-7-(4-cyano-3-fluorophenyl)imidazo[1,2-c]pyrimidin-8-yl)-2-methoxyphenoxy)-N-hydroxyheptanamide hydrochloride with a yield of 22.5%.

¹H NMR (400 MHZ, DMSO-d₆) δ ppm 10.35 (s, 1H), 8.24-8.19 (m, 3H), 7.99 (s, 1H), 7.87 (dd, J=8.2, 6.8 Hz, 1H), 7.55 (dd, J=10.6, 1.6 Hz, 1H), 7.33 (dd, J=8.2, 1.6 Hz, 1H), 7.03 (d, J=8.4 Hz, 1H), 6.97-6.75 (m, 2H), 4.08 (s, 2H), 3.81 (m, 5H), 3.41 (s, 1H), 3.23 (t, J=12.4 Hz, 2H), 2.09 (d, J=11.8 Hz, 2H), 2.00-1.79 (m, 4H), 1.70-1.39 (m, 4H), 1.41-1.17 (m, 4H).

ESI-MS (m/z)=602.3 [M+H]⁺.

The compounds of Examples 21-23 were prepared similarly according to the synthetic method of Example 20 (separation method 1), and the structure and characterization data are as follows:

| Ex- | | | | MS |
|---|---|---|---|---|
| am- | | | | (M + |
| ple | Chemical name | Structure | ¹H NMR | H)⁺ |
| 21 | 7-(4-(5-(4- Aminopiperidin-1-yl)- 7-(4-cyano-3- fluorophenyl)imidazo [1,2-c]pyrimidin-8-yl)- 2-hydroxyphenoxy)- N- hydroxyheptanamide hydrochloride | 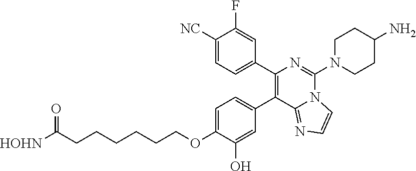 | ¹H NMR (400 MHz, Methanol- d₄)δ ppm 8.11 (d, J = 2.4 Hz, 1H), 7.91 (d, J = 2.4 Hz, 1H), 7.59 (dd, J = 8.2, 6.8 Hz, 1H), 7.44 (dd, J = 10.6, 1.6 Hz, 1H), 7.35 (dd, J = 8.0, 1.6 Hz, 1H), 6.97 (d, J = 8.4 Hz, 1H), 6.80- 6.58 (m, 2H), 4.16 (d, J = 10.6 Hz, 2H), 4.01 (t, J = 6.6 Hz, 2H), 3.45 (tt, J = 11.4, 4.2 Hz, 1H), 3.38-3.25 (m, 2H), 2.21- 1.85 (m, 6H), 1.76 (p, J = 6.8 Hz, 2H), 1.57 (dt, J = 7.6, 4.0 Hz, 2H), 1.52-1.39 (m, 2H), 1.39-1.27 (m, 2H). | 588.3 |
| 22 | 7-(4-(5-(5-(5-Amino- 2-azabicyclo [2.2.1]heptan- 2-yl)-7-(4-cyano- 3- fluorophenyl)imidazo [1,2-c]pyrimidin-8-yl)- 2-hydroxyphenoxy)- N- hydroxyheptanamide hydrochloride | 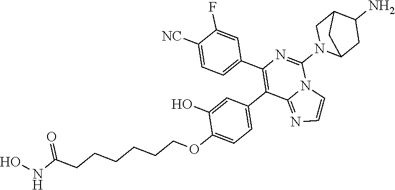 [SMILES: N#Cc1ccc(-c2nc(N3CC4CC3CC4N)n3ccnc3c2-c2ccc(OCCCCCCC(=O)NO)c(O)c2)cc1F] | ¹H NMR (400 MHz, Methanol- d₄) δ ppm 8.28 (dd, J = 5.0, 2.6 Hz, 1H), 7.87 (t, J = 2.4 Hz, 1H), 7.66 (m, 1H), 7.56-7.48 (m, 1H), 7.41 (m, 1H), 7.04 (d, J = 8.2 Hz, 1H), 6.87-6.67 (m, 2H), 4.98 (d, J = 11.2 Hz, 1H), 4.41- 4.28 (m, 1H), 4.10 (t, J = 6.6 Hz, 2H), 3.98-3.78 (m, 1H), 3.60 (dd, J = 7.6, 3.8 Hz, 1H), 3.20- 2.93 (m, 1H), 2.77 (dt, J = 13.2, 7.4 Hz, 1H), 2.42 (m, 1H), 2.21- 2.06 (m, 3H), 1.92-1.78 (m, 3H), 1.75-1.60 (m, 2H), 1.60- 1.48 (m, 2H), 1.42 (q, J = 7.8 Hz, 2H). | 600.3 |
| 23 | (E)-3-(4-(4-(5-(4- Aminopiperidin-1-yl)- 7-(4-cyano-3- fluorophenyl)imidazo [1,2-c]pyrimidin-8-yl)- 2- hydroxyphenoxy) methyl)phenyl)-N- hydroxyacrylamide hydrochloride | 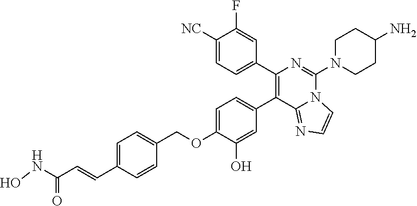 [SMILES: N#Cc1ccc(-c2nc(N3CCC(N)CC3)n3ccnc3c2-c2ccc(OCc3ccc(/C=C/C(=O)NO)cc3)c(O)c2)cc1F] | ¹H NMR (400 MHz, Methanol- d₄) δ 8.17 (d, J = 2.4 Hz, 1H), 7.98 (d, J = 2.4 Hz, 1H), 7.71- 7.44 (m, 7H), 7.38 (dd, J = 8.2, 1.6 Hz, 1H), 7.08 (d, J = 8.4 Hz, 1H), 6.85 (d, J = 2.2 Hz, 1H), 6.73 (dd, J = 8.2, 2.2 Hz, 1H), 6.49 (d, J = 15.9 Hz, 1H), 5.29 (s, 2H), 4.24 (d, J = 13.6 Hz, 2H), 3.53 (s, 1H), 3.40 (d, J = 12.6 Hz, 2H), 2.21 (d, J = 11.8 Hz, 2H), 1.98 (tt, J = 12.8, 6.8 Hz, 2H). | 620.2 |

### Example 24

### Preparation of 7-(4-(5-cyano-4-(4-cyano-3-fluorophenyl)-6-(4-(methylamino)piperidin-1-yl)pyridin-3-yl)-2-hydroxyphenoxy)-N-hydroxyheptanamide hydrochloride

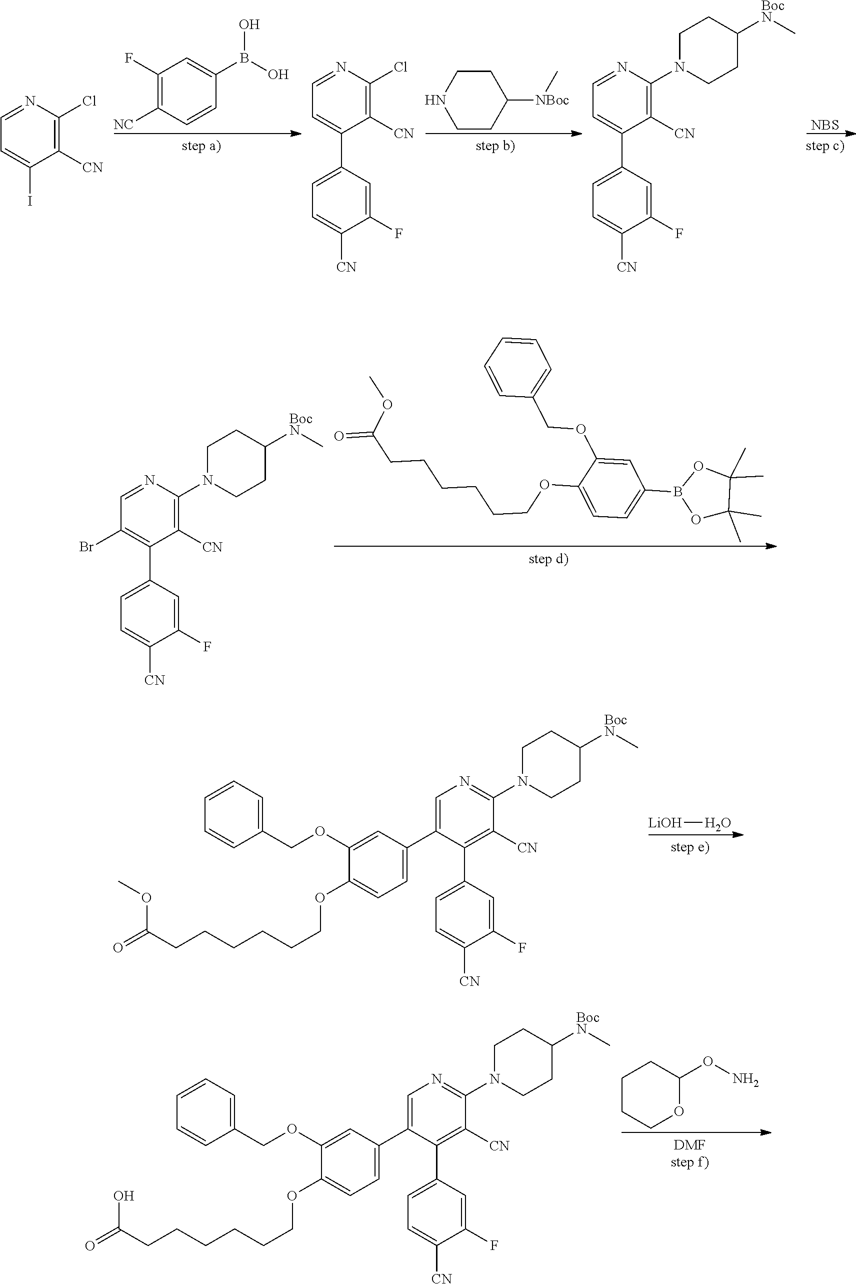

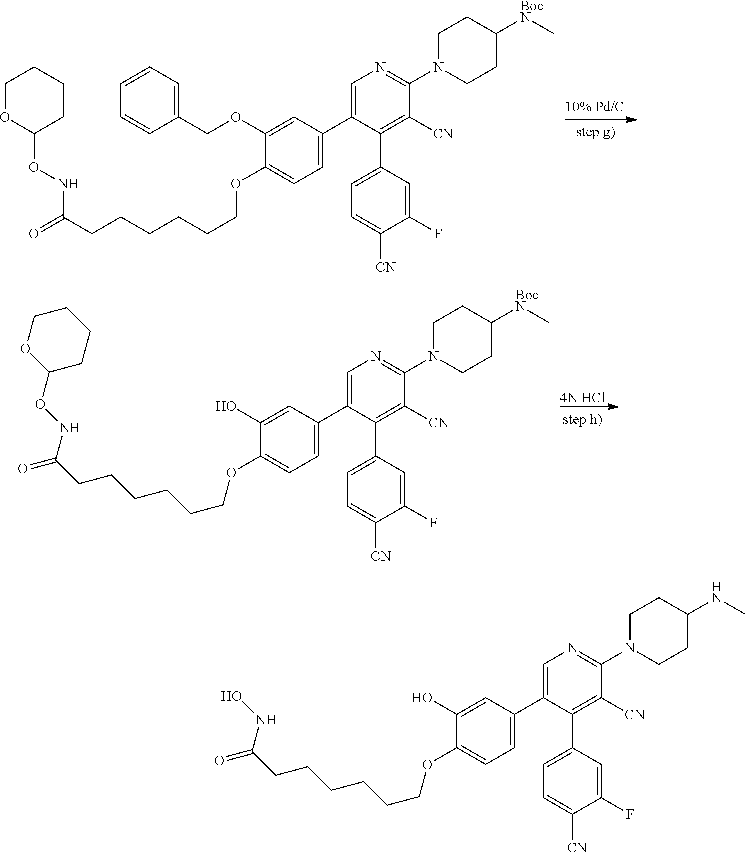

Step a): preparation of 2-chloro-4-(4-cyano-3-fluorophenyl)nicotinonitrile 2-Chloro-4-iodonicotinonitrile (1.0 g, 3.79 mmol), (4-cyano-3-fluorophenyl)boronic acid (688 mg, 4.17 mmol), Cs₂CO₃ (3.7 g, 11.37 mmol), Pd(dppf)Cl₂ (275 mg, 0.38 mmol), 1,4-dioxane (10 mL), and H₂O (2.5 mL) were added to a reaction flask and stirred at 100° C. for 1 hour. The reaction mixture was concentrated under reduced pressure, and the resultant residue was purified by column chromatography on silica gel (eluent: petroleum ether/ethyl acetate=3/1) to afford 2-chloro-4-(4-cyano-3-fluorophenyl)nicotinonitrile with a yield of 68.0%.

ESI-MS (m/z)=258.1 [M+H]⁺.

### Step b): preparation of tert-butyl (1-(3-cyano-4-(4-cyano-3-fluorophenyl)pyridin-2-yl)piperidin-4-yl)(methyl)carbamate

2-Chloro-4-(4-cyano-3-fluorophenyl)nicotinonitrile (660 mg, 2.58 mmol), tert-butyl piperidin-4-yl-carbamate (552 mg, 2.58 mmol), DIPEA (332 mg, 2.58 mmol), and NMP (10 mL) were added to a reaction flask and stirred at 130° C. for 1 hour. After the reaction was completed, water (20 mL) was added, followed by extraction with ethyl acetate (20 mL×3), and the organic phases were combined, washed with saturated brine (20 mL×2), and then concentrated to dryness under reduced pressure. The residue was purified by silica gel chromatography (eluent: petroleum ether/ethyl acetate=3/1) to afford tert-butyl (1-(3-cyano-4-(4-cyano-3-fluorophenyl)pyridin-2-yl)piperidin-4-yl)(methyl)carbamate with a yield of 68.5%.

ESI-MS (m/z)=436.2 [M+H]⁺.

### Step c): preparation of tert-butyl (1-(5-bromo-3-cyano-4-(4-cyano-3-fluorophenyl)pyridin-2-yl)piperidin-4-yl)(methyl)carbamate

Tert-butyl (1-(3-cyano-4-(4-cyano-3-fluorophenyl)pyridin-2-yl)piperidin-4-yl)(methyl)carbamate (600 mg, 1.38 mmol) and DMF (10 mL) were added to a reaction flask, and NBS (270 mg, 1.5 mmol) was added in portions under stirring in an ice-water bath, and the mixture was stirred at room temperature for 30 minutes. After the reaction was completed, water (40 mL) was added, followed by extraction with ethyl acetate (40 mL×3), and the organic phases were combined, washed with saturated brine (40 mL×2), and then concentrated to dryness under reduced pressure. The residue was purified by silica gel chromatography (eluent: petroleum ether/ethyl acetate=2/1) to afford tert-butyl (1-(5-bromo-3-cyano-4-(4-cyano-3-fluorophenyl)pyridin-2-yl)piperidin-4-yl)(methyl)carbamate with a yield of 77.0%.

ESI-MS (m/z)=514.1 [M+H]⁺.

### Step d): preparation of methyl 7-(2-(benzyloxy)-4-(6-(4-(tert-butoxycarbonyl)(methyl)amino)piperidin-1-yl)-5-cyano-4-(4-cyano-3-fluorophenyl)pyridin-3-yl)phenoxy)heptanoate

Tert-butyl (1-(5-bromo-3-cyano-4-(4-cyano-3-fluorophenyl)pyridin-2-yl)piperidin-4-yl)(methyl)carbamate (500 mg, 0.97 mmol), methyl 7-(2-(benzyloxy)-4-(4,4,5,5-tetramethyl-1,3,2-dioxaborolan-2-yl)phenoxy)heptanoate (501 mg, 1.07 mmol), Cs₂CO₃ (950.8 mg, 2.92 mmol), Pd(dppf)Cl₂ (70.6 mg, 0.1 mmol), 1,4-dioxane (10 mL), and H₂O (2.5 mL) were added to a reaction flask and stirred at 100° C. for 1 hour. The reaction liquid was concentrated to dryness under reduced pressure, and the residue was purified by silica gel chromatography (eluent: petroleum ether/ethyl acetate=3/1) to afford methyl 7-(2-(benzyloxy)-4-(6-(4-(tert-butoxycarbonyl)(methyl)amino)piperidin-1-yl)-5-cyano-4-(4-cyano-3-fluorophenyl)pyridin-3-yl)phenoxy)heptanoate with a yield of 68.0%.

ESI-MS (m/z)=776.4 [M+H]⁺.

### Step e): preparation of 7-(2-(benzyloxy)-4-(6-(4-((tert-butoxycarbonyl)(methyl)amino)piperidin-1-yl)-5-cyano-4-(4-cyano-3-fluorophenyl)pyridin-3-yl)phenoxy)heptanoic acid

Methyl 7-(2-(benzyloxy)-4-(6-(4-(tert-butoxycarbonyl)(methyl)amino)piperidin-1-yl)-5-cyano-4-(4-cyano-3-fluorophenyl)pyridin-3-yl)phenoxy)heptanoate (500 mg, 0.66 mmol) was dissolved in a solution of tetrahydrofuran (6 mL) and water (1 mL), and lithium hydroxide (95.3 mg, 3.97 mmol) was added, and the mixture was reacted at room temperature for 3 hours, then the pH of the solution was adjusted to 2˜3 with 1N HCl, then the mixture was extracted with ethyl acetate, and the organic phases were dried and concentrated under vacuum. The residue was used directly for the subsequent step.

ESI-MS (m/z)=762.4 [M+H]⁺.

### Step f): preparation of tert-butyl (1-(5-(3-(benzyloxy)-4-((7-oxo-7-((tetrahydro-2H-pyran-2-yl)oxy)amino)heptyl)oxy)phenyl)-3-cyano-4-(4-cyano-3-fluorophenyl)pyridin-2-yl)piperidin-4-yl)(methyl)carbamate

7-(2-(Benzyloxy)-4-(6-(4-((tert-butoxycarbonyl)(methyl)amino)piperidin-1-yl)-5-cyano-4-(4-cyano-3-fluorophenyl)pyridin-3-yl)phenoxy)heptanoic acid (186 mg, 0.24 mmol) and O-(tetrahydro-2H-pyran-2-yl)hydroxylamine (84.35 mg, 0.72 mmol) were dissolved in N,N-dimethylformamide (4 mL), followed by adding N,N-diisopropylethylamine (309.6 mg, 2.4 mmol) and HATU (118.63 mg, 0.31 mmol), and the mixture was reacted for 30 minutes, then water (60 mL) was added to quench the reaction, followed by extraction with ethyl acetate (60 mL×2), The organic phases were combined, washed with saturated brine (45 mL×2), dried over anhydrous sodium sulfate, filtered, and concentrated to obtain a crude product. The residue was then purified by silica gel chromatography (eluent: dichloromethane/ethyl acetate=10/7) to afford tert-butyl (1-(5-(3-(benzyloxy)-4-((7-oxo-7-((tetrahydro-2H-pyran-2-yl)oxy)amino)heptyl)oxy)phenyl)-3-cyano-4-(4-cyano-3-fluorophenyl)pyridin-2-yl)piperidin-4-yl)(methyl)carbamate with a yield of 96%.

ESI-MS m/z=861.4 [M+H]⁺.

### Step g): preparation of tert-butyl (1-(3-cyano-4-(4-cyano-3-fluorophenyl)-5-(3-hydroxy-4-((7-oxo-7-((tetrahydro-2H-pyran-2-yl)oxy)amino)heptyl)oxy)phenyl)pyridin-2-yl)piperidin-4-yl)(methyl)carbamate

Tert-butyl (1-(5-(3-(benzyloxy)-4-((7-oxo-7-((tetrahydro-2H-pyran-2-yl)oxy)amino)heptyl)oxy)phenyl)-3-cyano-4-(4-cyano-3-fluorophenyl)pyridin-2-yl)piperidin-4-yl)(methyl)carbamate (200 mg, 0.23 mmol) was dissolved in ethyl acetate (10 mL), and 10% palladium on carbon (50 mg) was added. The mixture was purged with hydrogen and reacted for 30 minutes, and the reaction liquid was then filtered to obtain a filtrate and the filtrate was concentrated to obtain a crude product, which was directly used in the next reaction step.

ESI-MS m/z=771.4 [M+H]⁺.

### Step h): preparation of 7-(4-(5-cyano-4-(4-cyano-3-fluorophenyl)-6-(4-(methylamino)piperidin-1-yl)pyridin-3-yl)-2-hydroxyphenoxy)-N-hydroxyheptanamide

Tert-butyl (1-(3-cyano-4-(4-cyano-3-fluorophenyl)-5-(3-hydroxy-4-((7-oxo-7-((tetrahydro-2H-pyran-2-yl)oxy)amino)heptyl)oxy)phenyl)pyridin-2-yl)piperidin-4-yl)(methyl)carbamate (200 mg, 0.26 mmol) was dissolved in 4N HCl/EA (5 mL) and the reaction was conducted for 30 minutes, and the mixture was concentrated to obtain a crude product, and purified by Prep-HPLC (separation method 1) to afford 7-(4-(5-cyano-4-(4-cyano-3-fluorophenyl)-6-(4-(methylamino)piperidin-1-yl)pyridin-3-yl)-2-hydroxyphenoxy)-N-hydroxyheptanamide hydrochloride with a yield of 24%.

¹H NMR (400 MHZ, Methanol-d₄) δ ppm 8.31 (s, 1H), 7.67 (t, J=7.2 Hz, 1H), 7.31 (d, J=9.6 Hz, 1H), 7.18 (d, J=7.8 Hz, 1H), 6.72 (d, J=8.2 Hz, 1H), 6.48-6.36 (m, 2H), 4.35 (d, J=13.2 Hz, 2H), 3.88 (t, J=6.6 Hz, 2H), 3.21 (m, 3H), 2.67 (s, 3H), 2.29-2.05 (m, 4H), 1.73 (ddt, J=16.8, 13.2, 7.4 Hz, 4H), 1.55 (dp, J=12.6, 7.2 Hz, 2H), 1.36 (dp, J=12.2, 8.2, 7.2 Hz, 4H).

ESI-MS m/z=587.3 [M+H]⁺.

Examples 25-26 were prepared similarly according to the synthetic method of Example 24 (separation method 1), and the structure and characterization data are as follows:

| Ex- | | | | MS |
|---|---|---|---|---|
| am- | | | | (M + |
| ple | Chemical name | Structure | ¹H NMR | H)⁺ |
| 25 | 7-(4-(6-(4- Aminopiperidin-1-yl)- 5-cyano-4-(4-cyano-3- fluorophenyl)pyridin-3- yl)-2-hydroxyphenoxy)- N-hydroxyheptanamide hydrochloride | 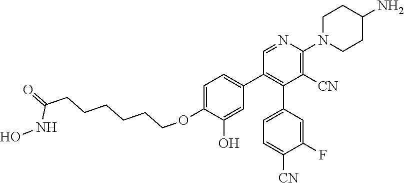 [SMILES: N#Cc1ccc(-c2c(-c3ccc(OCCCCCCC(=O)NO)c(O)c3)cnc(N3CCC(N)CC3)c2C#N)cc1F] | ¹H NMR (400 MHz, Methanol- d₄) δ 8.45 (s, 1H), 7.77 (dd, J = 7.8, 6.6 Hz, 1H), 7.40 (dd, J = 9.6, 1.4 Hz, 1H), 7.26 (dd, J = 8.0, 1.4 Hz, 1H), 6.83 (d, J = 8.2 Hz, 1H), 6.66-6.36 (m, 2H), 4.42 (d, J = 13.4 Hz, 2H), 4.00 (t, J = 6.4 Hz, 2H), 3.45 (ddt, J = 11.4, 8.6, 4.2 Hz, 1H), 3.25 (d, J = 11.8 Hz, 2H), 2.26-2.07 (m, 4H), 1.84 (ddd, J = 2.2, 12.2, 5.4 Hz, 4H), 1.66 (h, J = 7.2 Hz, 2H), 1.56-1.35 (m, 4H). | 573.3 |
| 26 | 7-(4-(6-(4- Aminopiperidin-1-yl)- 5-cyano-4-(4-cyano-3- fluorophenyl)-2- methylpyridin-3-yl)-2- hydroxyphenoxy)-N- hydroxyheptanamide hydrochloride | 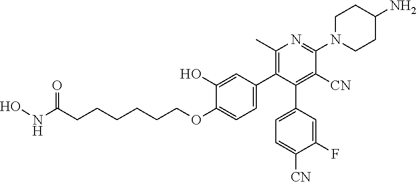 [SMILES: Cc1nc(N2CCC(N)CC2)c(C#N)c(-c2ccc(C#N)c(F)c2)c1-c1ccc(OCCCCCCC(=O)NO)c(O)c1] | ¹H NMR (400 MHz, Methanol- d₄) δ ppm 7.66 (t, J = 7.4 Hz, 1H), 7.26 (d, J = 9.8 Hz, 1H), 7.17 (dd, J = 8.0, 1.4 Hz, 1H), 6.79 (d, J = 8.2 Hz, 1H), 6.49 (d, J = 2.0 Hz, 1H), 6.43 (dd, J = 8.2, 2.2 Hz, 1H), 4.40 (d, J = 13.4 Hz, 2H), 3.97 (t, J = 6.4 Hz, 2H), 3.41 (td, J = 11.2, 5.4 Hz, 1H), 3.25- 3.06 (m, 2H), 2.33 (s, 3H), 2.13 (q, J = 8.0 Hz, 4H), 1.80 (qd, J = 15.6, 13.9, 5.4 Hz, 4H), 1.64 (p, J = 7.4 Hz, 2H), 1.56-1.34 (m, 4H). | 587.3 |

### Example 21

### Preparation of 7-(4-(6-(4-aminopiperidin-1-yl)-2-(4-cyano-3-fluorophenyl)-4-methoxypyridin-3-yl)-2-hydroxyphenoxy)-N-hydroxyheptanamide hydrochloride

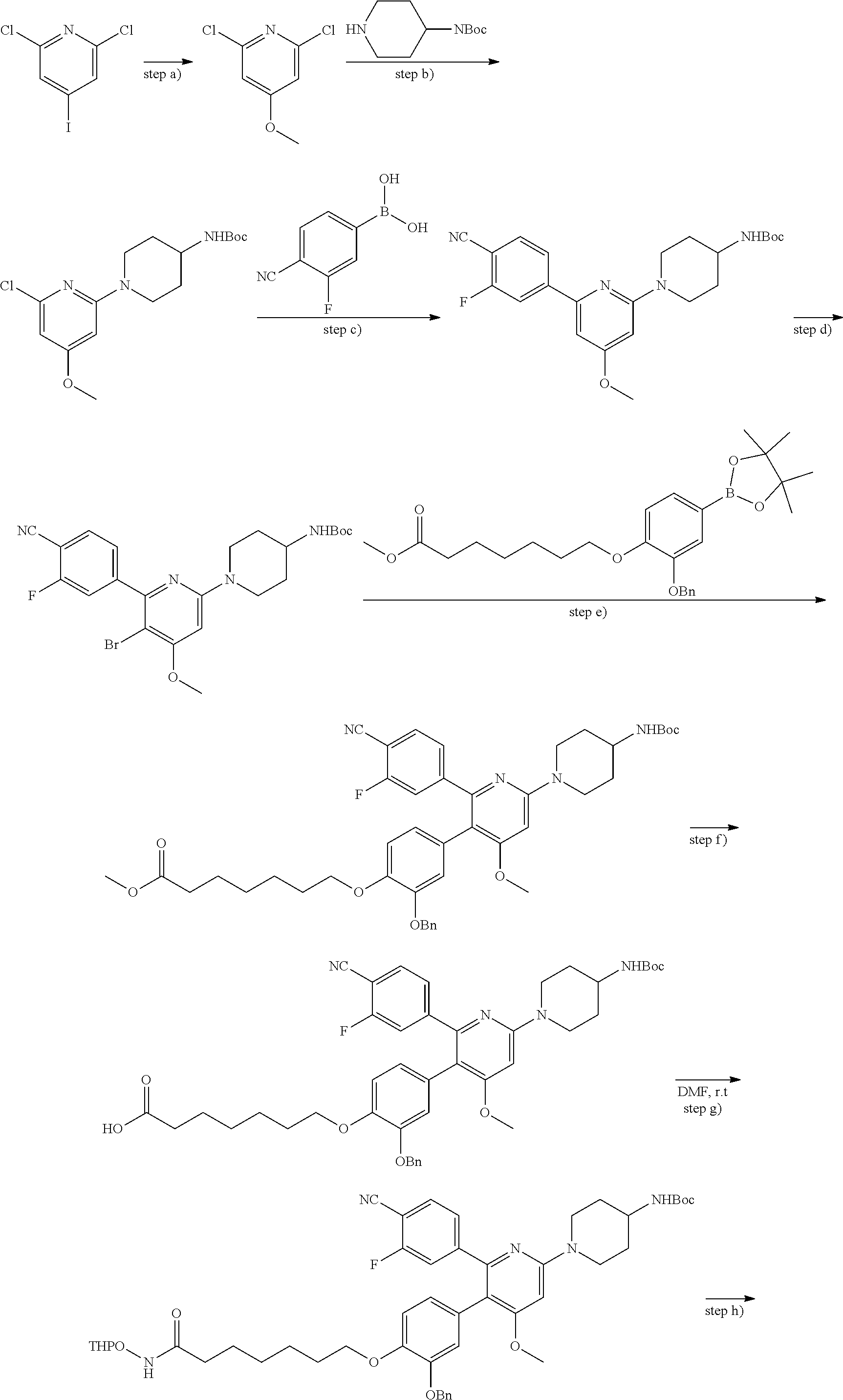

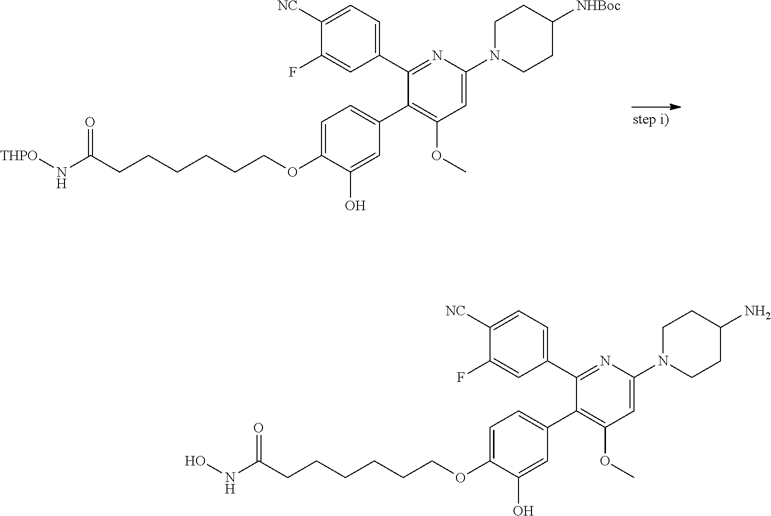

### Step a): preparation of 2,6-dichloro-4-methoxypyridine

4-(Benzyloxy)-2,6-dichloropyridine (2 g, 11.049 mmol) and MeOH (20 mL) were added to a reaction flask and stirred at room temperature for 16 hours. After the reaction was completed, water (100 mL) was added, followed by extraction with ethyl acetate (100 mL×3). The organic phases were combined, washed with saturated brine (100 mL×2), and then concentrated to dryness under reduced pressure. The residue was purified by silica gel chromatography (eluent: petroleum ether/ethyl acetate=2/1) to afford 2,6-dichloro-4-methoxypyridine with a yield of 60.5%.

¹H NMR (400 MHZ, Chloroform-d) δ ppm 6.79 (s, 2H), 3.87 (s, 3H).

ESI-MS (m/z)=178.0 [M+H]⁺.

### Step b): preparation of tert-butyl (1-(6-chloro-4-methoxypyridin-2-yl)piperidin-4-yl)carbamate

2,6-Dichloro-4-methoxypyridine (1.2 g, 6.63 mmol), tert-butyl piperidin-4-yl-carbamate (2.7 g, 13.26 mmol), and NMP (15 mL) were added to a microwave reactor and stirred at 130° C. for 2 hours. After the reaction was completed, water (100 mL) was added, followed by extraction with ethyl acetate (100 mL×3), and the organic phases were combined, washed with saturated brine (100 mL×2), and then concentrated to dryness under reduced pressure. The residue was purified by silica gel chromatography (eluent: petroleum ether/ethyl acetate=1/1) to afford tert-butyl (1-(6-chloro-4-methoxypyridin-2-yl)piperidin-4-yl)carbamate with a yield of 52.0%.

ESI-MS (m/z)=342.5 [M+H]⁺.

### Step c): preparation of tert-butyl (1-(6-(4-cyano-3-fluorophenyl)-4-methoxypyridin-2-yl)piperidin-4-yl)carbamate

Tert-butyl (1-(6-chloro-4-methoxypyridin-2-yl)piperidin-4-yl)carbamate (1.2 g, 3.45 mmol), (4-cyano-3-fluorophenyl)boronic acid (853 mg, 5.17 mmol), Cs₂CO₃ (2.2 g, 6.9 mmol), Pd(dppf)Cl₂ (253 mg, 0.35 mmol), 1,4-dioxane (10 mL), and H₂O (2.5 mL) were added to a reaction flask and stirred at 120° C. for 1 hour. The reaction liquid was concentrated to dryness under reduced pressure. The residue was then purified by silica gel chromatography (eluent: petroleum ether/ethyl acetate=2/3) to afford tert-butyl (1-(6-(4-cyano-3-fluorophenyl)-4-methoxypyridin-2-yl)piperidin-4-yl)carbamate with a yield of 73.0%.

¹H NMR (400 MHz, DMSO-d₆) δ 8.12 (dd, J=15.4, 9.8 Hz, 2H), 7.02 (d, J=1.8 Hz, 1H), 6.83 (d, J=7.8 Hz, 1H), 6.40 (s, 1H), 4.33 (d, J=13.2 Hz, 2H), 3.86 (m, 3H), 3.31 (s, 3H), 2.94 (t, J=12.4 Hz, 2H), 1.80 (d, J=12.0 Hz, 2H), 1.39 (s, 9H).

ESI-MS (m/z)=427.2 [M+H]⁺.

### Step d): preparation of tert-butyl (1-(5-bromo-6-(4-cyano-3-fluorophenyl)-4-methoxypyridin-2-yl)piperidin-4-yl)carbamate

Tert-butyl (1-(6-(4-cyano-3-fluorophenyl)-4-methoxypyridin-2-yl)piperidin-4-yl)carbamate (1.1 g, 2.52 mmol) and DMF (20 mL) were added to a reaction flask, and NBS (448 mg, 2.516 mmol) was added in portions under stirring in an ice bath, and the mixture was stirred at room temperature for 30 minutes. After the reaction was completed, water (40 mL) was added, followed by extraction with ethyl acetate (40 mL×3), and the organic phases were combined, washed with saturated brine (40 mL×2), and then concentrated to dryness under reduced pressure. The residue was purified by silica gel chromatography (eluent: petroleum ether/ethyl acetate=2/3) to afford tert-butyl (1-(5-bromo-6-(4-cyano-3-fluorophenyl)-4-methoxypyridin-2-yl)piperidin-4-yl)carbamate with a yield of 72.0%.

ESI-MS (m/z)=505.2 [M+H]⁺.

### Step e): preparation of methyl 7-(2-(benzyloxy)-4-(6-(4-(tert-butoxycarbonyl)amino)piperidin-1-yl)-2-(4-cyano-3-fluorophenyl)-4-methoxypyridin-3-yl)phenoxy)heptanoate

Tert-butyl (1-(5-bromo-6-(4-cyano-3-fluorophenyl)-4-methoxypyridin-2-yl)piperidin-4-yl)carbamate (200 mg, 397 μmol), methyl 7-(2-(benzyloxy)-4-(4,4,5,5-tetramethyl-1,3,2-dioxaborolan-2-yl)phenoxy)heptanoate (279 mg, 596 μmol), Cs₂CO₃ (259 mg, 794 μmol), Pd(dppf)Cl₂ (29 mg, 39.7 μmol), 1,4-dioxane (10 mL), and H₂O (2.5 mL) were added to a reaction flask and the mixture was stirred at 120° C. for 1 hour. The reaction liquid was concentrated to dryness under reduced pressure. The residue was then purified by silica gel chromatography (eluent: petroleum ether/ethyl acetate=1/2) to afford methyl 7-(2-(benzyloxy)-4-(6-(4-(tert-butoxycarbonyl)amino)piperidin-1-yl)-2-(4-cyano-3-fluorophenyl)-4-methoxypyridin-3-yl)phenoxy)heptanoate with a yield of 56.0%.

ESI-MS (m/z)=767.4 [M+H]⁺.

### Step f): preparation of 7-(2-(benzyloxy)-4-(6-(4-(tert-butoxycarbonyl)amino)piperidin-1-yl)-2-(4-cyano-3-fluorophenyl)-4-methoxypyridin-3-yl)phenoxy)heptanoic acid

Methyl 7-(2-(benzyloxy)-4-(6-(4-(tert-butoxycarbonyl)amino)piperidin-1-yl)-2-(4-cyano-3-fluorophenyl)-4-methoxypyridin-3-yl)phenoxy)heptanoate (171 mg, 222 μmol), lithium hydroxide monohydrate (43 mg, 1.1 mmol), tetrahydrofuran (2 mL), isopropanol (2 mL), and water (1 mL) were added to a reaction flask and stirred at room temperature for 12 hours. Under stirring in an ice bath, 1N hydrochloric acid was added dropwise to adjust the pH to 3˜4, and water (10 mL) was added, followed by extraction with ethyl acetate (10 mL×3). The organic phases were combined, washed with saturated brine (10 mL×2), and then concentrated to dryness under reduced pressure to afford 7-(2-(benzyloxy)-4-(6-(4-(tert-butoxycarbonyl)amino)piperidin-1-yl)-2-(4-cyano-3-fluorophenyl)-4-methoxypyridin-3-yl)phenoxy)heptanoic acid, which was used directly for the subsequent step.

ESI-MS (m/z)=753.4 [M+H]⁺.

### Step g): preparation of tert-butyl (1-(5-(3-(benzyloxy)-4-((7-oxo-7-((tetrahydro-2H-pyran-2-yl)oxy)amino)heptyl)oxy)phenyl)-6-(4-cyano-3-fluorophenyl)-4-methoxypyridin-2-yl)piperidin-4-yl)carbamate

7-(2-(Benzyloxy)-4-(6-(4-(tert-butoxycarbonyl)amino)piperidin-1-yl)-2-(4-cyano-3-fluorophenyl)-4-methoxypyridin-3-yl)phenoxy)heptanoic acid (167 mg, 222 μmol), O-(tetrahydro-2H-pyran-2-yl)hydroxylamine (52 mg, 444 μmol), DIEA (53 mg, 444 μmol), and DMF (2 mL) were added to a reaction flask, and the mixture was stirred at room temperature, and HATU (114 mg, 300 μmol) was added. The reaction was maintained at room temperature for 1 hour. After the reaction was completed, water (10 mL) was added to quench the reaction, followed by extraction with ethyl acetate (20 mL×2). The organic phases were combined, sequentially washed with saturated sodium bicarbonate aqueous solution (20 mL×1) and saturated brine (10 mL×2), dried over anhydrous sodium sulfate, filtered, and the filtrate was concentrated under reduced pressure. The residue was purified by silica gel chromatography (eluent: petroleum ether/ethyl acetate=1/1) to afford tert-butyl (1-(5-(3-(benzyloxy)-4-((7-oxo-7-((tetrahydro-2H-pyran-2-yl)oxy)amino)heptyl)oxy)phenyl)-6-(4-cyano-3-fluorophenyl)-4-methoxypyridin-2-yl)piperidin-4-yl)carbamate with a yield of 64.7%.

ESI-MS (m/z)=852.4 [M+H]⁺.

### Step h): preparation of tert-butyl (1-(6-(4-cyano-3-fluorophenyl)-5-(3-hydroxy-4-((7-oxo-7-((tetrahydro-2H-pyran-2-yl)oxy)amino)heptyl)oxy)phenyl)-4-methoxypyridin-2-yl)piperidin-4-yl)carbamate

Tert-butyl (1-(5-(3-(benzyloxy)-4-((7-oxo-7-((tetrahydro-2H-pyran-2-yl)oxy)amino)heptyl)oxy)phenyl)-6-(4-cyano-3-fluorophenyl)-4-methoxypyridin-2-yl)piperidin-4-yl)carbamate (122 mg, 143 μmol), palladium on carbon (25 mg, 5%), and ethanol (5 mL) were sequentially added to a reaction flask, and the mixture was purged with hydrogen three times and stirred at room temperature for 2 hours under hydrogen atmosphere. After the reaction was completed, it was filtered, and the filtrate was concentrated under reduced pressure to afford tert-butyl (1-(6-(4-cyano-3-fluorophenyl)-5-(3-hydroxy-4-((7-oxo-7-((tetrahydro-2H-pyran-2-yl)oxy)amino)heptyl)oxy)phenyl)-4-methoxypyridin-2-yl)piperidin-4-yl)carbamate with a yield of 87.0%.

ESI-MS (m/z)=762.4 [M+H]⁺.

### Step i): preparation of 7-(4-(6-(4-aminopiperidin-1-yl)-2-(4-cyano-3-fluorophenyl)-4-methoxypyridin-3-yl)-2-hydroxyphenoxy)-N-hydroxyheptanamid hydrochloride

Tert-butyl (1-(6-(4-cyano-3-fluorophenyl)-5-(3-hydroxy-4-((7-oxo-7-((tetrahydro-2H-pyran-2-yl)oxy)amino)heptyl)oxy)phenyl)-4-methoxypyridin-2-yl)piperidin-4-yl)carbamate (95 mg, 124 μmol) was added to a reaction flask, followed by adding hydrogen chloride solution in ethyl acetate (4M, 2.5 mL), and the mixture was stirred at room temperature for 1 hour, resulting in the precipitation of a significant amount of solid, After concentrating under reduced pressure, the crude product was purified by Prep-HPLC (separation method 1) to afford 7-(4-(6-(4-aminopiperidin-1-yl)-2-(4-cyano-3-fluorophenyl)-4-methoxypyridin-3-yl)-2-hydroxyphenoxy)-N-hydroxyheptanamide hydrochloride with a yield of 34.0%.

¹H NMR (400 MHZ, Methanol-d₄) δ 7.63 (t, J=7.0 Hz, 1H), 7.39 (d, J=9.4 Hz, 1H), 7.24 (d, J=7.8 Hz, 1H), 6.79-6.57 (m, 2H), 6.49 (s, 1H), 6.36 (d, J=7.8 Hz, 1H), 4.26 (d, J=13.0 Hz, 2H), 3.95 (s, 3H), 3.88 (t, J=6.4 Hz, 2H), 3.46 (s, 1H), 3.32 (t, J=12.8 Hz, 2H), 2.14 (d, J=12.2 Hz, 2H), 2.03 (t, J=7.4 Hz, 2H), 1.84-1.62 (m, 4H), 1.57-1.50 (m, 2H), 1.44-1.26 (m, 4H).

ESI-MS (m/z)=578.3 [M+H]⁺.

Examples 28-30 were prepared similarly according to the synthetic method of Example 27 (the separation method for the compound: hydrochloride and formate were prepared by separation method 1 and 3, respectively), and the structures and characterization data are as follows:

| Ex- | | | | MS |
|---|---|---|---|---|
| am- | | | | (M + |
| ple | Chemical name | Structure | ¹H NMR | H)⁺ |
| 28 | (E)-3-(4-(6-(4-Amino- piperidin-1-yl)-2-(4- cyano-3-fluorophenyl)-4- methoxypyridin-3-yl)-2- hydroxyphenyl)-N- hydroxyacrylamide formate | 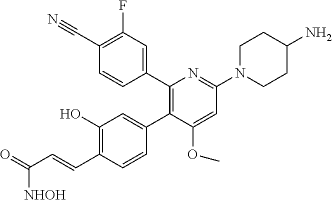 | ¹H NMR (400 MHz, Methanol-d₄) δ 8.54 (s, 3H), 7.78 (d, J = 15.7 Hz, 1H), 7.51 (t, J = 7.4 Hz, 1H), 7.35- 7.26 (m, 2H), 7.19 (d, J = 7.8 Hz, 1H), 6.67-6.45 (m, 3H), 4.57 (d, J = 11.8 Hz, 2H), 3.84 (s, 3H), 3.37 (s, 1H), 3.02 (t, J = 12.1 Hz, 2H), 2.08 (s, 2H), 1.65 (s, 2H). | 504.2 |
| 29 | (E)-4-(4-(6-(4-Amino- piperidin-1-yl)-2-(4- cyano-3-fluorophenyl)-4- methoxypyridin-3-yl)-2- hydroxyphenoxy)-N- hydroxybut-2-enamide hydrochloride | 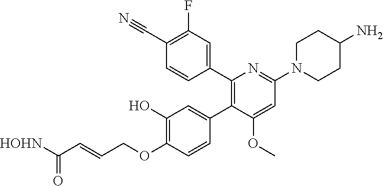 | ¹H NMR (400 MHz, Methanol-d₄) δ 7.72 (dd, J = 8.0, 6.6 Hz, 1H), 7.50 (dd, J = 9.6, 1.4 Hz, 1H), 7.32 (dd, J = 8.0, 1.4 Hz, 1H), 6.94 (dt, J = 15.6, 4.2 Hz, 1H), 6.79 (d, J = 7.8 Hz, 2H), 6.61 (d, J = 2.0 Hz, 1H), 6.47 (dd, J = 8.2, 2.0 Hz, 1H), 6.14 (dt, J = 15.4, 2.0 Hz, 1H), 4.74 (dd, J = 4.6, 1.8 Hz, 2H), 4.36 (d, J = 13.8 Hz, 2H), 4.04 (s, 3H), 3.55 (tt, J = 10.2, 4.4 Hz, 1H), 3.47-3.36 (m, 2H), 2.33-2.13 (m, 2H), 1.87 (qd, J = 12.4, 3.8 Hz, 2H). | 534.2 |
| 30 | (E)-3-(4-((2-(5-(6-(4- Aminopiperidin- 1-yl)-2-(4-cyano- 3-fluorophenyl)-4- methoxypyridin-3-yl)-2- methoxyphenoxy)ethyl) amino)methyl)phenyl)-N- hydroxyacrylamide hydrochloride | 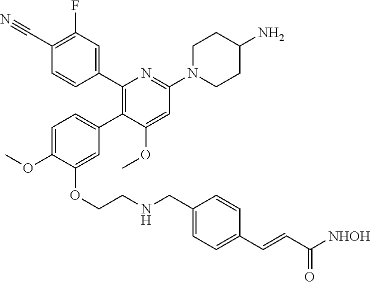 | ¹H NMR (400 MHz, Methanol-d₄) δ ppm 7.70-7.63 (m, 3H), 7.62- 7.55 (m, 3H), 7.42 (d, J = 10.0 Hz, 1H), 7.33 (dd, J = 8.2, 1.4 Hz, 1H), 6.94 (d, J = 8.4 Hz, 1H), 6.81 (d, J = 2.0 Hz, 1H), 6.76-6.67 (m, 2H), 6.54 (d, J = 15.8 Hz, 1H), 4.45 (d, J = 13.8 Hz, 2H), 4.37 (s, 2H), 4.18 (t, J = 4.6 Hz, 2H), 3.96 (s, 3H), 3.85 (s, 3H), 3.51 (d, J = 4.2 Hz, 1H), 3.45 (t, J = 4.8 Hz, 2H), 3.29- 3.22 (m, 2H), 2.26-2.11 (m, 2H), 1.81 (qd, J = 12.0, 4.0 Hz, 2H). | 667.3 |

### Example 31

### Preparation of 7-((2-(4-aminopiperidin-1-yl)-6-(4-cyano-3-fluorophenyl)pyridin-4-yl)oxy)-N-hydroxyheptanamide hydrochloride

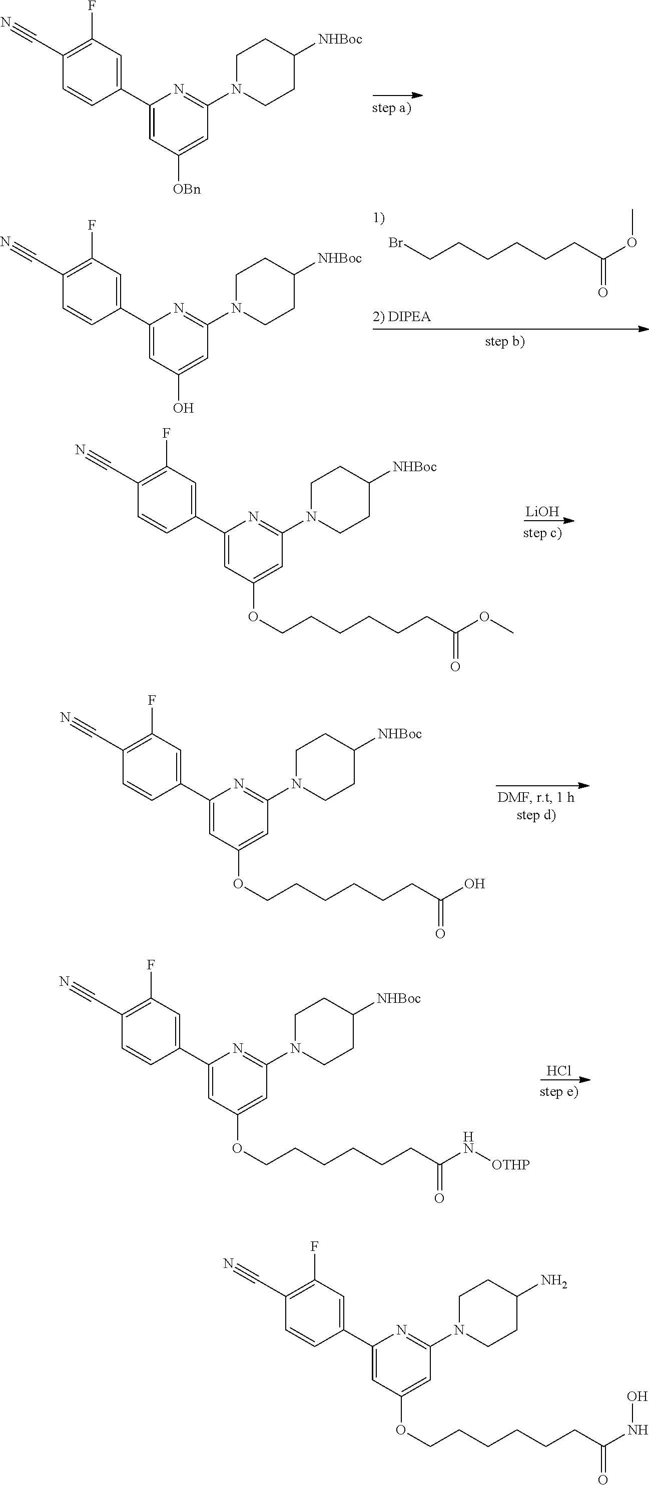

### Step a): preparation of tert-butyl (1-(6-(4-cyano-3-fluorophenyl)-4-hydroxypyridin-2-yl)piperidin-4-yl)carbamate

The product of Step b) in Example 1: tert-butyl (1-(4-(benzyloxy)-6-(4-cyano-3-fluorophenyl)pyridin-2-yl)piperidin-4-yl)carbamate (600 mg, 1.2 mmol) was dissolved in dichloromethane (12 mL), the mixture was cooled to 0° C., then 1 N boron tribromide solution in dichloromethane (5 mL) was added dropwise, and the reaction was maintained at 0° C. for 1 hour, then the reaction was quenched with water, and the pH of the solution was adjusted to 8-9 with sodium bicarbonate. Then extracted with ethyl acetate (30 mL×3), the organic phases were then combined and washed with saturated brine, dried over sodium sulphate and concentrated under reduced pressure. The residue was purified by column chromatography on silica gel (eluent: petroleum ether:ethyl acetate=1:1) to afford tert-butyl (1-(6-(4-cyano-3-fluorophenyl)-4-hydroxypyridin-2-yl)piperidin-4-yl)carbamate with a yield of 80%. ESI-MS m/z=413.2 [M+H]⁺.

### Step b): preparation of f methyl 7-((2-(4-((tert-butoxycarbonyl)amino)piperidin-1-yl)-6-(4-cyano-3-fluorophenyl)pyridin-4-yl)oxy)heptanoate hydrochloride

Tert-butyl (1-(6-(4-cyano-3-fluorophenyl)-4-hydroxypyridin-2-yl)piperidin-4-yl)carbamate (400 mg, 0.97 mmol) was dissolved in acetonitrile (4 mL), methyl 7-bromoheptanoate (1.3 g, 5.82 mmol) and N,N-diisopropylethylamine (752.2 mg, 5.82 mmol) were added at room temperature. The mixture was purged with nitrogen, heated to 90° C. and reacted overnight, and the reaction was monitored by LCMS, which indicated that the starting material was completely consumed. After the reaction liquid was cooled to room temperature, water (5 mL) was added, followed by extraction with ethyl acetate (5 mL×2), and the organic phases were combined, washed with saturated brine (5 mL×2), dried with anhydrous sodium sulfate, filtered, and concentrated under reduced pressure. The residue was purified by silica gel chromatography (eluent: petroleum ether:ethyl acetate=2:1) to afford methyl 7-((2-(4-((tert-butoxycarbonyl)amino)piperidin-1-yl)-6-(4-cyano-3-fluorophenyl)pyridin-4-yl)oxy)heptanoate with a yield of 77.0%.

ESI-MS m/z=555.3 [M+H]⁺.

### Step c): preparation of 7-((2-(4-((tert-butoxycarbonyl)amino)piperidin-1-yl)-6-(4-cyano-3-fluorophenyl)pyridin-4-yl)oxy)heptanoic acid

Lithium hydroxide (129.6 mg, 5.4 mmol) was dissolved in a mixture of tetrahydrofuran:water=2:1 (11 mL), then, methyl 7-((2-(4-((tert-butoxycarbonyl)amino)piperidin-1-yl)-6-(4-cyano-3-fluorophenyl)pyridin-4-yl)oxy)heptanoate (300 mg, 0.54 mmol) was added to the lithium hydroxide solution. The mixture was purged with nitrogen and stirred at room temperature overnight, and the reaction was monitored by LCMS, which indicated that the starting material was completely consumed. Water (20 mL) was added, and the pH was adjusted to 3-4 with 1N hydrochloric acid solution, and the mixture was extracted with ethyl acetate (20 mL×2), and the organic phases were combined, washed with saturated brine (15 mL×2), then dried over anhydrous sodium sulfate, filtered, and concentrated to obtain the crude product 7-((2-(4-((tert-butoxycarbonyl)amino)piperidin-1-yl)-6-(4-cyano-3-fluorophenyl)pyridin-4-yl)oxy)heptanoic acid, which was used directly for the subsequent step. ESI-MS m/z=541.3 [M+H]⁺.

### Step d): preparation of tert-butyl (1-(6-(4-cyano-3-fluorophenyl)-4-((7-oxo-7-(((tetrahydro-2H-pyran-2-yl)oxy)amino)heptyl)oxy)pyridin-2-yl)piperidin-4-yl)carbamate

7-((2-(4-((Tert-butoxycarbonyl)amino)piperidin-1-yl)-6-(4-cyano-3-fluorophenyl)pyridin-4-yl)oxy)heptanoic acid (300 mg, 0.47 mmol) was dissolved in DMF (5 mL) and stirred at room temperature for ten minutes, then, diisopropylethylamine (179.8 mg, 1.41 mmol), O-(tetrahydro-2H-pyran-2-yl)hydroxylamine (65 mg, 0.56 mmol), and hexafluorophosphate (211.8 mg, 0.56 mmol) were added, the reaction liquid was purged with nitrogen and stirred at room temperature for one hour, and the reaction was monitored by LCMS, which indicated that the starting material was completely consumed. After the reaction liquid was cooled to room temperature, water (5 mL) was added, followed by extraction with ethyl acetate (5 mL×2). The organic phases were combined, washed with saturated brine (5 mL×2), dried over anhydrous sodium sulfate, filtered, and concentrated under reduced pressure. The residue was purified by silica gel chromatography (eluent: petroleum ether:ethyl acetate=2:1) to afford tert-butyl (1-(6-(4-cyano-3-fluorophenyl)-4-((7-oxo-7-((tetrahydro-2H-pyran-2-yl)oxy)amino)heptyl)oxy)pyridin-2-yl)piperidin-4-yl)carbamate, which was used directly for the subsequent reaction.

ESI-MS m/z=640.3 [M+H]⁺.

### Step e): preparation of 7-((2-(4-aminopiperidin-1-yl)-6-(4-cyano-3-fluorophenyl)pyridin-4-yl)oxy)-N-hydroxyheptanamide

Ethyl acetate (10 mL) was added to tert-butyl (1-(6-(4-cyano-3-fluorophenyl)-4-((7-oxo-7-(((tetrahydro-2H-pyran-2-yl)oxy)amino)heptyl)oxy)pyridin-2-yl)piperidin-4-yl)carbamate (200 mg, 0.31 mmol), followed by adding hydrochloric acid (11.47 mg, 0.31 mmol), then the mixture was stirred at room temperature for one hour, and the reaction was monitored by LCMS, which indicated that the starting material was completely consumed. After the reaction solution was cooled to room temperature, it was concentrated under reduced pressure. The residue was then purified by Prep-HPLC (separation method 1) to afford 7-((2-(4-aminopiperidin-1-yl)-6-(4-cyano-3-fluorophenyl)pyridin-4-yl)oxy)-N-hydroxyheptanamide hydrochloride with a yield of 31%.

¹H NMR (400 MHZ, DMSO-d₆) δ 10.36 (s, 1H), 8.34-7.90 (m, 2H), 7.62 (s, 1H), 7.08 (d, J=10.0 Hz, 1H), 6.45 (s, 2H), 4.45 (d, J=13.2 Hz, 2H), 4.11 (s, 2H), 3.29 (s, 1H), 2.92 (d, J=13.2 Hz, 2H), 1.96 (m, 4H), 1.72 (s, 2H), 1.59-1.26 (m, 8H).

ESI-MS m/z=456.2 [M+H]⁺.

### Example 32

### Preparation of 7-(4-(5-(3-amino-8-azabicyclo[3.2.1]octane-8-carbonyl)-3-(4-cyano-3-fluorophenyl)thiophen-2-yl)-2-hydroxyphenoxy)-N-hydroxyheptanamide hydrochloride

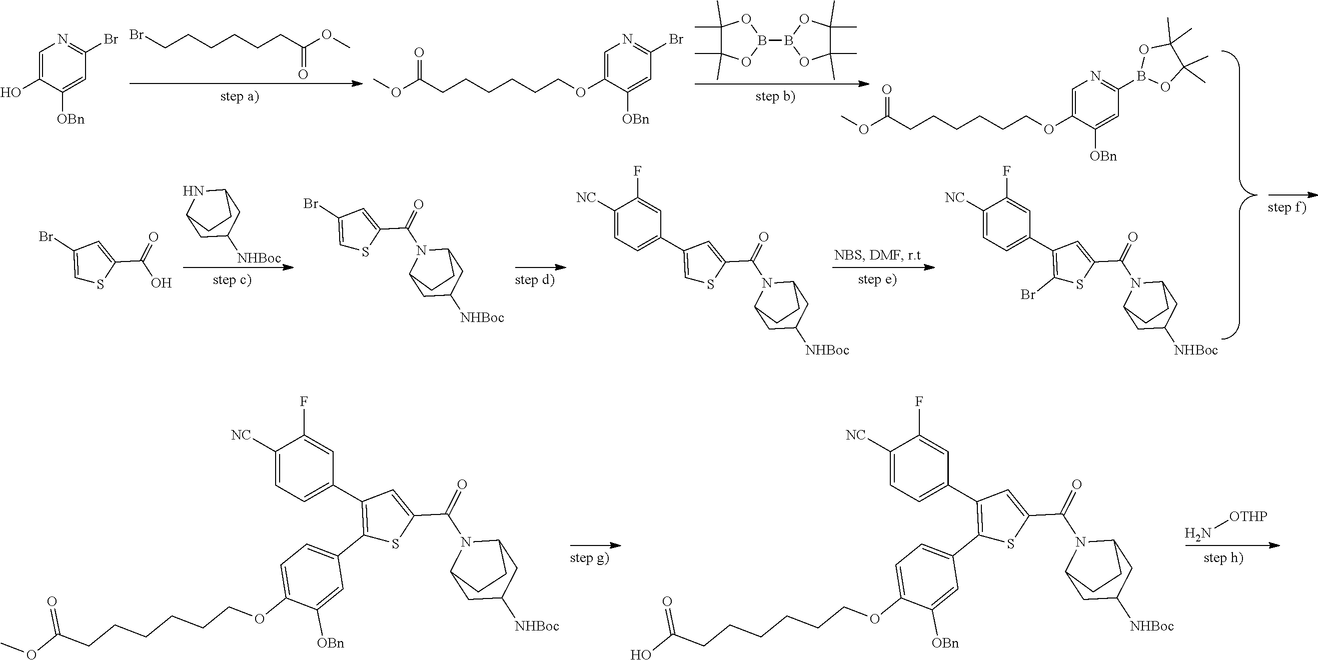

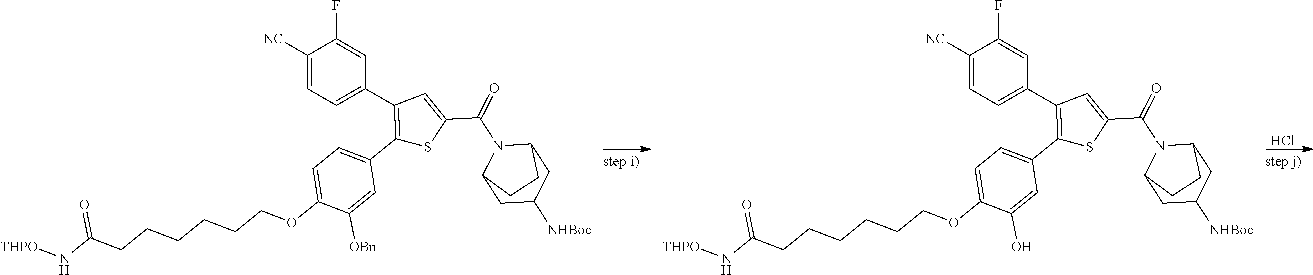

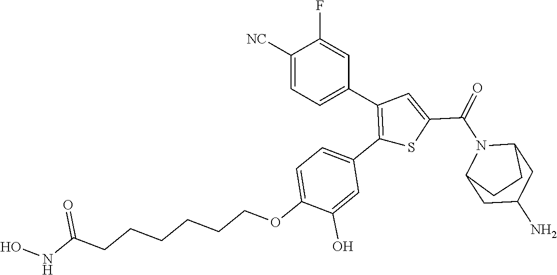 [SMILES: N#Cc1ccc(-c2cc(C(=O)N3C4CCC3CC(N)C4)sc2-c2ccc(OCCCCCCC(=O)NO)c(O)c2)cc1F]

### Step a): preparation of methyl 7-(2-(benzyloxy)-4-bromophenoxy)heptanoate

2-(Benzyloxy)-4-bromophenol (2 g, 7.16 mmol), methyl 7-bromoheptanoate (2.39 g, 10.74 mmol), and DIEA (3.7 g, 28.64 mmol) were added to a reaction flask containing acetonitrile (20 mL), and the mixture was stirred at 90° C. for 16 hours. The reaction solution was concentrated after adding silica gel and the residue was purified by silica gel chromatography (eluent: petroleum ether/ethyl acetate=1/1) to afford methyl 7-(2-(benzyloxy)-4-bromophenoxy)heptanoate with a yield of 70%.

ESI-MS m/z=421.1 [M+H]⁺.

### Step b): preparation of methyl 7-(2-(benzyloxy)-4-(4,4,5,5-tetramethyl-1,3,2-dioxaborolan-2-yl)phenoxy)heptanoate

Methyl 7-(2-(benzyloxy)-4-bromophenoxy)heptanoate (2 g, 4.75 mmol), bis(pinacolato)diboron (2.41 g, 9.5 mmol), Pd(dppf)Cl₂ (0.35 g, 0.48 mmol), and potassium acetate (0.93 g, 9.5 mmol) were added to a reaction flask containing 1,4-dioxane (20 mL), and the mixture was stirred at 110° C. for 3 hours. The reaction solution was concentrated after adding silica gel and the residue was purified by silica gel chromatography (eluent: petroleum ether/ethyl acetate=3/1) to afford methyl 7-(2-(benzyloxy)-4-(4,4,5,5-tetramethyl-1,3,2-dioxaborolan-2-yl)phenoxy)heptanoate with a yield of 72%.

ESI-MS m/z=469.3 [M+H]⁺.

### Step c): Preparation of tert-butyl (8-(4-bromothiophene-2-carbonyl)-8-azabicyclo[3.2.1]octan-3-yl)carbamate

4-Bromothiophene-2-carboxylic acid (3 g, 14.47 mmol), HATU (8.25 g, 21.71 mmol), DIEA (5.61 g, 43.41 mmol), and tert-butyl (8-azabicyclo[3.2.1]octan-3-yl)carbamate (3.27 g, 14.47 mmol) were added to a reaction flask containing DMF (30 mL), and the mixture was stirred at room temperature for 2 hours. After the reaction was completed, water (20 mL) was added to quench the reaction, and the mixture was extracted with ethyl acetate (40 mL×2). The organic phases were combined, washed with saturated brine (40 mL×2), and then dried over anhydrous sodium sulfate. After filtering, the residue was purified by silica gel chromatography (eluent: petroleum ether/ethyl acetate=1/1) to afford tert-butyl (8-(4-bromothiophene-2-carbonyl)-8-azabicyclo[3.2.1]octan-3-yl)carbamate with a yield of 90%.

ESI-MS m/z=415.1 [M+H]⁺.

### Step d): preparation of tert-butyl (8-(4-(4-cyano-3-fluorophenyl)thiophene-2-carbonyl)-8-azabicyclo[3.2.1]octan-3-yl)carbamate

Tert-butyl (8-(4-bromothiophene-2-carbonyl)-8-azabicyclo[3.2.1]octan-3-yl)carbamate (5.5 g, 13.24 mmol), 4-cyano-3-fluorophenylboronic acid (3.28 g, 19.86 mmol), Pd(dppf)Cl₂ (0.97 g, 1.32 mmol), and cesium carbonate (8.63 g, 26.48 mmol) were added to a microwave tube containing 1,4-dioxane (40 mL) and water (8 mL), and the mixture was stirred at 70° C. for 1 hour. The reaction liquid was concentrated by adding silica gel and the residue was purified by silica gel chromatography (eluent: petroleum ether/ethyl acetate=1/1) to afford tert-butyl (8-(4-(4-cyano-3-fluorophenyl)thiophene-2-carbonyl)-8-azabicyclo[3.2.1]octan-3-yl)carbamate with a yield of 98%.

ESI-MS m/z=456.2 [M+H]⁺.

### Step e): preparation of tert-butyl (8-(5-bromo-4-(4-cyano-3-fluorophenyl)thiophene-2-carbonyl)-8-azabicyclo[3.2.1]octan-3-yl)carbamate

Tert-butyl (8-(4-(4-cyano-3-fluorophenyl)thiophene-2-carbonyl)-8-azabicyclo[3.2.1]octan-3-yl)carbamate (4 g, 8.78 mmol) and NBS (1.72 g, 9.66 mmol) were added to a reaction flask containing DMF (40 mL), and the mixture was stirred at 60° C. for 2 hours. Water (20 mL) was added to quench the reaction, and the mixture was extracted with ethyl acetate (40 mL×2). The organic phases were combined, washed with saturated brine (40 mL×2), dried over anhydrous sodium sulfate. After filtering, the solution was concentrated, and the residue was purified by silica gel chromatography (eluent: petroleum ether/ethyl acetate=1/1) to afford tert-butyl (8-(5-bromo-4-(4-cyano-3-fluorophenyl)thiophene-2-carbonyl)-8-azabicyclo[3.2.1]octan-3-yl)carbamate with a yield of 51%.

ESI-MS m/z=534.1 [M+H]⁺.

### Step f): preparation of methyl 7-(2-(benzyloxy)-4-(5-(3-((tert-butoxycarbonyl)amino)-8-azabicyclo[3.2.1]octane-8-carbonyl)-3-(4-cyano-3-fluorophenyl)thiophen-2-yl)phenoxy)heptanoate

Methyl 7-(2-(benzyloxy)-4-(4,4,5,5-tetramethyl-1,3,2-dioxaborolan-2-yl)phenoxy)heptanoate (200 mg, 0.43 mmol), tert-butyl (8-(5-bromo-4-(4-cyano-3-fluorophenyl)thiophene-2-carbonyl)-8-azabicyclo[3.2.1]octan-3-yl)carbamate (0.23 g, 0.43 mmol), Pd(dppf)Cl₂ (63 mg, 0.086 mmol), and cesium carbonate (0.28 g, 0.86 mmol) were added to a reaction solution containing 1,4-dioxane (6 mL) and water (1.5 mL), and the mixture was stirred under microwave heating at 120° C. for 1 hour. The reaction solution was concentrated after adding silica gel and the residue was purified by silica gel chromatography (eluent: petroleum ether/ethyl acetate=1/1) to afford methyl 7-(2-(benzyloxy)-4-(5-(3-((tert-butoxycarbonyl)amino)-8-azabicyclo[3.2.1]octane-8-carbonyl)-3-(4-cyano-3-fluorophenyl)thiophen-2-yl)phenoxy)heptanoate with a yield of 70%.

ESI-MS m/z=796.3 [M+H]⁺.

### Step g): preparation of 7-(2-(benzyloxy)-4-(5-(3-((tert-butoxycarbonyl)amino)-8-azabicyclo[3.2.1]octane-8-carbonyl)-3-(4-cyano-3-)fluorophenyl)thiophen-2-yl)phenoxy)heptanoic acid

Methyl 7-(2-(benzyloxy)-4-(5-(3-((tert-butoxycarbonyl)amino)-8-azabicyclo[3.2.1]octane-8-carbonyl)-3-(4-cyano-3-fluorophenyl)thiophen-2-yl)phenoxy)heptanoate (300 mg, 0.38 mmol) and lithium hydroxide (0.16 g, 3.8 mmol) were added to a reaction flask containing THF (10 mL), MeOH (6 mL), and water (2 mL), and the mixture was stirred at room temperature for 2 hours. Ice-water mixture (10 mL) was added to the reaction solution, followed by the adjustment of pH to 3 with 2M HCl, and the mixture was then extracted with a mixture of DCM/MeOH (5:1) (20 mL×2). The organic phases were combined, washed with saturated brine (15 mL×2), dried over anhydrous sodium sulfate, filtered, and concentrated to afford 7-(2-(benzyloxy)-4-(5-(3-((tert-butoxycarbonyl)amino)-8-azabicyclo[3.2.1]octane-8-carbonyl)-3-(4-cyano-3-)fluorophenyl)thiophen-2-yl)phenoxy)heptanoic acid with a yield of 62%.

ESI-MS m/z=782.3 [M+H]⁺.

### Step h): preparation of tert-butyl (8-(5-(3-(benzyloxy)-4-((7-oxo-7-((tetrahydro-2H-pyran-2-yl)oxy)amino)heptyl)oxy)phenyl)-4-(4-cyano-3-fluorophenyl)thiophene-2-carbonyl)-8-azabicyclo[3.2.1]octan-3-yl)carbamate

7-(2-(Benzyloxy)-4-(5-(3-((tert-butoxycarbonyl)amino)-8-azabicyclo[3.2.1]octane-8-carbonyl)-3-(4-cyano-3-)fluorophenyl)thiophen-2-yl)phenoxy)heptanoic acid (130 mg, 0.17 mmol), HATU (4.5 mg, 65 μmol), O-(tetrahydro-2H-pyran-2-yl)hydroxylamine (97 mg, 0.26 mmol), and DIEA (66 mg, 0.51 mmol) were added to a reaction flask containing DMF (10 mL), and the mixture was stirred at room temperature for 2 hours. Water (10 mL) was added to quench the reaction, followed by extraction with ethyl acetate (20 mL×2). The organic phases were combined, washed with saturated brine (15 mL×2), dried over anhydrous sodium sulfate, and concentrated under reduced pressure. The residue was purified by silica gel chromatography (eluent: petroleum ether/ethyl acetate=1/1) to afford tert-butyl (8-(5-(3-(benzyloxy)-4-((7-oxo-7-((tetrahydro-2H-pyran-2-yl)oxy)amino)heptyl)oxy)phenyl)-4-(4-cyano-3-fluorophenyl)thiophene-2-carbonyl)-8-azabicyclo[3.2.1]octan-3-yl)carbamate with a yield of 67%. ESI-MS m/z=881.4 [M+H]⁺.

### Step i): preparation of tert-butyl (8-(4-(4-cyano-3-fluorophenyl)-5-(3-hydroxy-4-((7-oxo-7-(((tetrahydro-2H-pyran-2-yl)oxy)))amino)heptyl)oxy)phenyl)thiophene-2-carbonyl)-8-azabicyclo[3.2.1]octan-3-yl)carbamate

Tert-butyl (8-(5-(3-(benzyloxy)-4-((7-oxo-7-((tetrahydro-2H-pyran-2-yl)oxy)amino)heptyl)oxy)phenyl)-4-(4-cyano-3-fluorophenyl)thiophene-2-carbonyl)-8-azabicyclo[3.2.1]octan-3-yl)carbamate (100 mg, 0.11 mmol) and palladium on carbon (21 mg, 0.11 mmol) were added to a reaction flask containing ethyl acetate (5 mL), and the mixture was stirred at 40° C. for 36 hours under hydrogen atmosphere. The reaction solution was filtered with diatomaceous earth and concentrated to afford tert-butyl (8-(4-(4-cyano-3-fluorophenyl)-5-(3-hydroxy-4-((7-oxo-7-(((tetrahydro-2H-pyran-2-yl)oxy)))amino)heptyl)oxy)phenyl)thiophene-2-carbonyl)-8-azabicyclo[3.2.1]octan-3-yl)carbamate with a yield of 75%.

ESI-MS m/z=791.3 [M+H]⁺.

### Step j): Preparation of 7-(4-(5-(3-amino-8-azabicyclo[3.2.1]octane-8-carbonyl)-3-(4-cyano-3-fluorophenyl)thiophen-2-yl)-2-hydroxyphenoxy)-N-hydroxyheptanamide hydrochloride

4.0M Hydrochloric acid solution in EA (3 mL) was added to a reaction flask containing tert-butyl (8-(4-(4-cyano-3-fluorophenyl)-5-(3-hydroxy-4-((7-oxo-7-(((tetrahydro-2H-pyran-2-yl)oxy)))amino)heptyl)oxy)phenyl)thiophene-2-carbonyl)-8-azabicyclo[3.2.1]octan-3-yl)carbamate (85 mg, 0.11 mmol), and the mixture was stirred at room temperature for 1 hour. The mixture was concentrated and then purified by Prep-HPLC (separation method 1) to afford 7-(4-(5-(3-amino-8-azabicyclo[3.2.1]octane-8-carbonyl)-3-(4-cyano-3-fluorophenyl)thiophen-2-yl)-2-hydroxyphenoxy)-N-hydroxyheptanamide hydrochloride with a yield of 16%.

¹H NMR (400 MHZ, DMSO-d₆) δ ppm 10.32 (s, 1H), 9.16 (t, J=8.0 Hz, 1H), 8.63 (s, 1H), 7.88 (t, J=7.6 Hz, 2H), 7.65 (s, 1H), 7.57 (dd, J=10.8, 1.4 Hz, 1H), 7.27 (dd, J=8.0, 1.6 Hz, 1H), 6.92 (d, J=8.6 Hz, 1H), 6.70 (d, J=6.8 Hz, 2H), 4.77 (s, 2H), 3.95 (t, J=6.6 Hz, 2H), 3.62 (s, 1H), 2.12-1.89 (m, 6H), 1.72 (dq, J=21.8, 7.8, 6.8 Hz, 6H), 1.51 (p, J=7.4 Hz, 2H), 1.41 (p, J=7.4 Hz, 2H), 1.30 (q, J=8.0 Hz, 2H).

ESI-MS m/z=607.2 [M+H]⁺.

### Example 33

### Preparation of (E)-3-(4-(((1-(4-(4-cyano-3-fluorophenyl)-5-(3-hydroxy-4-methoxyphenyl)thiophene-2-carbonyl)piperidin-4-yl)amino)methyl)phenyl)-N-hydroxyacrylamide hydrochloride

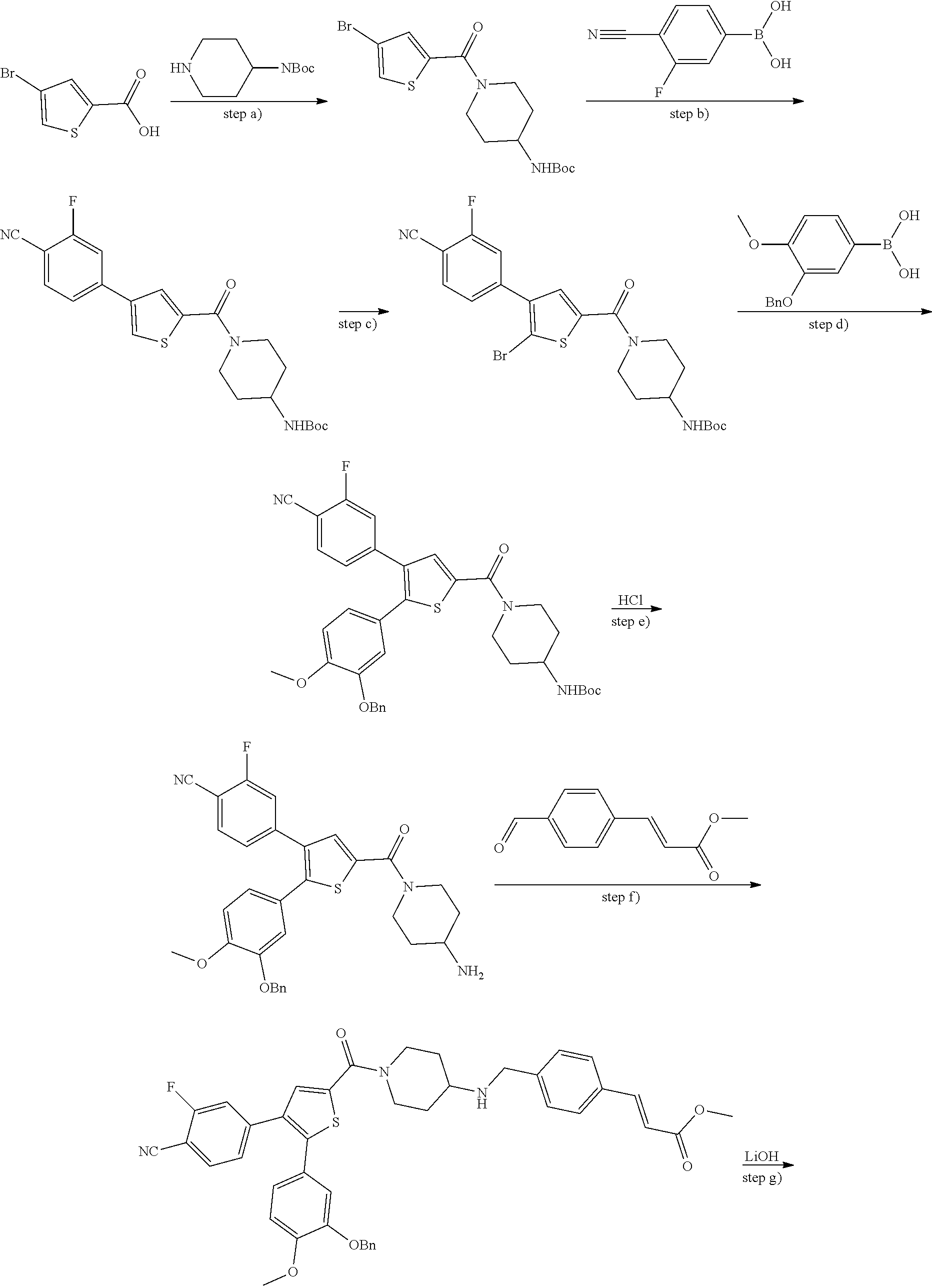

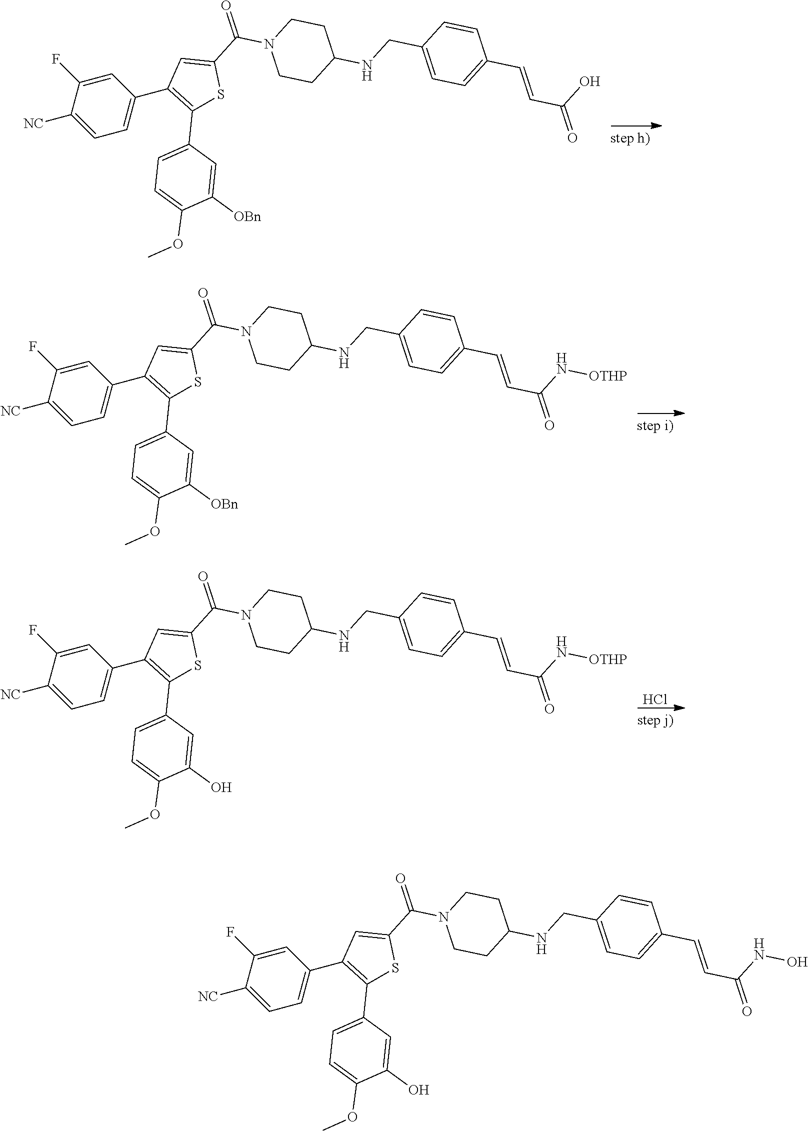

### Step a): Preparation of tert-butyl (1-(4-bromothiophene-2-carbonyl)piperidin-4-yl)carbamate

4-Bromothiophene-2-carboxylic acid (1 g, 4.82 mmol), tert-butyl piperidin-4-yl-carbamate (1.16 g, 5.78 mmol), HATU (2.75 g, 7.23 mmol), and DIEA (1.87 g, 14.46 mmol) were added to a reaction flask containing DMF (20 mL), and the mixture was stirred at room temperature for 2 hours. Water (20 mL) was added to quench the reaction, followed by extraction with ethyl acetate (20 mL×2). The organic phases were combined, washed with saturated brine (20 mL×2), then dried over anhydrous sodium sulfate, filtered. The residue was purified by silica gel chromatography (eluent: petroleum ether/ethyl acetate=1/1) to afford tert-butyl (1-(4-bromothiophene-2-carbonyl)piperidin-4-yl)carbamate with a yield of 91%.

ESI-MS m/z=389.1 [M+H]⁺.

### Step b): preparation of tert-butyl (1-(4-(4-cyano-3-fluorophenyl)thiophene-2-carbonyl)piperidin-4-yl)carbamate

Tert-butyl (1-(4-bromothiophene-2-carbonyl)piperidin-4-yl)carbamate (1.6 g, 4.11 mmol), (4-cyano-3-fluorophenyl)boronic acid (0.81 g, 4.93 mmol), Pd(dppf)Cl₂ (0.30 g, 0.41 mmol), and cesium carbonate (2.68 g, 8.22 mmol) were added to a microwave tube containing 1,4-dioxane (10 mL) and water (2 mL), and the mixture was stirred under microwave heating at 120° C. for 45 minutes. The reaction solution was concentrated after adding silica gel and the residue was purified by silica gel chromatography (eluent: petroleum ether/ethyl acetate=1/1) to afford tert-butyl (1-(4-(4-cyano-3-fluorophenyl)thiophene-2-carbonyl)piperidin-4-yl)carbamate with a yield of 99%. ESI-MS m/z=430.2 [M+H]⁺.

### Step c): preparation of tert-butyl (1-(5-bromo-4-(4-cyano-3-fluorophenyl)thiophene-2-carbonyl)piperidin-4-yl)carbamate

Tert-butyl (1-(4-(4-cyano-3-fluorophenyl)thiophene-2-carbonyl)piperidin-4-yl)carbamate (1.7 g, 3.96 mmol) and NBS (1.06 g, 5.94 mmol) were added to a reaction flask containing DMF (15 mL), and the mixture was stirred at 60° C. for 2 hours. The reaction solution was quenched with water (20 mL), then extracted with ethyl acetate (20 mL×2). The organic phases were combined, washed with saturated brine (15 mL×2), dried over anhydrous sodium sulfate, and concentrated under vacuum. The residue was purified by silica gel chromatography (eluent: petroleum ether/ethyl acetate=1/1) to afford tert-butyl (1-(5-bromo-4-(4-cyano-3-fluorophenyl)thiophene-2-carbonyl)piperidin-4-yl)carbamate with a yield of 98%.

ESI-MS m/z=508.1 [M+H]⁺.

### Step d): Preparation of tert-butyl (1-(5-(3-(benzyloxy)-4-methoxyphenyl)-4-(4-cyano-3-fluorophenyl)thiophene-2-carbonyl)piperidin-4-yl)carbamate

Tert-butyl (1-(5-bromo-4-(4-cyano-3-fluorophenyl)thiophene-2-carbonyl)piperidin-4-yl)carbamate (1.7 g, 3.34 mmol), (3-(benzyloxy)-4-methoxyphenyl)boronic acid (1.29 g, 5.01 mmol), Pd(dppf)Cl₂ (0.24 g, 0.33 mmol), and cesium carbonate (2.18 g, 6.68 mmol) were added to a microwave tube containing 1,4-dioxane (14 mL) and water (2 mL), and the mixture was stirred at 120° C. for 1 hour. The reaction solution was concentrated after adding silica gel, and the residue was purified by silica gel chromatography (eluent: petroleum ether/ethyl acetate=1/1) to afford tert-butyl (1-(5-(3-(benzyloxy)-4-methoxyphenyl)-4-(4-cyano-3-fluorophenyl)thiophene-2-carbonyl)piperidin-4-yl)carbamate with a yield of 93%.

ESI-MS m/z=642.2 [M+H]⁺.

### Step e): preparation of 4-(5-(4-aminopiperidine-1-carbonyl)-2-(3-(benzyloxy)-4-methoxyphenyl)thiophen-3-yl)-2-fluorobenzonitrile

Tert-butyl (1-(5-(3-(benzyloxy)-4-methoxyphenyl)-4-(4-cyano-3-fluorophenyl)thiophene-2-carbonyl)piperidin-4-yl)carbamate (900 mg, 1.40 mmol) and 2M hydrochloric acid solution in ethyl acetate (10 mL) were added to a reaction flask and the mixture was stirred at room temperature for 1 hour. The reaction solution was concentrated to afford 4-(5-(4-aminopiperidine-1-carbonyl)-2-(3-(benzyloxy)-4-methoxyphenyl)thiophen-3-yl)-2-fluorobenzonitrile with a yield of 92%.

ESI-MS m/z=542.2 [M+H]⁺.

### Step f): preparation of methyl (E)-3-(4-(((1-(5-(3-(benzyloxy)-4-methoxyphenyl)-4-(4-cyano-3-fluorophenyl)thiophene-2-carbonyl)piperidin-4-yl)amino)methyl)phenyl)acrylate

4-(5-(4-Aminopiperidine-1-carbonyl)-2-(3-(benzyloxy)-4-methoxyphenyl)thiophen-3-yl)-2-fluorobenzonitrile (680 mg, 1.26 mmol) and methyl (E)-3-(4-formylphenyl)acrylate (0.24 g, 1.26 mmol) were added to a reaction flask containing DCE (10 mL), and the mixture was stirred at room temperature for 2 hours. After the complete consumption of the starting materials monitored by LC-MS, sodium cyanoborohydride (0.32 g, 5.04 mmol) was added to thesolution at an ice bath, and the mixture was further stirred at room temperature for 2 hours. The reaction was quenched by adding ice-water mixture of saturated sodium bicarbonate, then extracted with ethyl acetate (20 mL×2). The organic phases were combined, washed with saturated brine (15 mL×2), dried over anhydrous sodium sulfate, filtered, and the residue was purified by silica gel chromatography (eluent: petroleum ether/ethyl acetate=1/1) to afford methyl (E)-3-(4-(((1-(5-(3-(benzyloxy)-4-methoxyphenyl)-4-(4-cyano-3-fluorophenyl)thiophene-2-carbonyl)piperidin-4-yl)amino)methyl)phenyl)acrylate with a yield of 72%.

ESI-MS m/z=716.3 [M+H]⁺.

### Step g): preparation of (E)-3-(4-(((1-(5-(3-(benzyloxy)-4-methoxyphenyl)-4-(4-cyano-3-fluorophenyl)thiophene-2-carbonyl)piperidin-4-yl)amino)methyl)phenyl)acrylic acid

Methyl (E)-3-(4-(((1-(5-(3-(benzyloxy)-4-methoxyphenyl)-4-(4-cyano-3-fluorophenyl)thiophene-2-carbonyl)piperidin-4-yl)amino)methyl)phenyl)acrylate (630 mg, 0.88 mmol) and lithium hydroxide (0.21 g, 8.8 mmol) were added to a reaction flask containing THF (5 mL), MeOH (3 mL), and water (2 mL), and the mixture was stirred at room temperature for 2 hours. The solution was acidified to pH 4 by adding 2M hydrochloric acid, then extracted with a mixture of MeOH/DCM (1:5) (20 mL×2). The organic phases were combined, washed with saturated brine (15 mL×2), dried over anhydrous sodium sulfate, and concentrated to afford (E)-3-(4-(((1-(5-(3-(benzyloxy)-4-methoxyphenyl)-4-(4-cyano-3-fluorophenyl)thiophene-2-carbonyl)piperidin-4-yl)amino)methyl)phenyl)acrylic acid with a yield of 97%.

ESI-MS m/z=702.2 [M+H]⁺.

### Step h): preparation of (E)-3-(4-(((1-(5-(3-(benzyloxy)-4-methoxyphenyl)-4-(4-cyano-3-fluorophenyl)thiophene-2-carbonyl)piperidin-4-yl)amino)methyl)phenyl)-N-((tetrahydro-2H-pyran-2-yl)oxy) acrylamide

(E)-3-(4-(((1-(5-(3-(Benzyloxy)-4-methoxyphenyl)-4-(4-cyano-3-fluorophenyl)thiophene-2-carbonyl)piperidin-4-yl)amino)methyl)phenyl)acrylic acid (600 mg, 0.85 mmol), O-(tetrahydro-2H-pyran-2-yl)hydroxylamine (0.20 g, 1.71 mmol), HATU (0.39 g, 1.02 mmol), and DIEA (0.33 g, 2.55 mmol) were added to a reaction flask containing DMF (10 mL), and the mixture was stirred at room temperature for 2 hours. The reaction liquid was quenched with water (10 mL), then extracted with ethyl acetate (20 mL×2). The organic phases were combined, washed with saturated brine (15 mL×2), dried over anhydrous sodium sulfate, and concentrated under reduced pressure. The residue was purified with silica gel chromatography (eluent: DCM/MeOH=10/1) to afford (E)-3-(4-(((1-(5-(3-(benzyloxy)-4-methoxyphenyl)-4-(4-cyano-3-fluorophenyl)thiophene-2-carbonyl)piperidin-4-yl)amino)methyl)phenyl)-N-((tetrahydro-2H-pyran-2-yl)oxy) acrylamide with a yield of 44%.

ESI-MS m/z=801.3 [M+H]⁺.

### Step i): preparation of (E)-3-(4-(((1-(4-(4-cyano-3-fluorophenyl)-5-(3-hydroxy-4-methoxyphenyl)thiophene-2-carbonyl)piperidin-4-yl)amino)methyl)phenyl)-N-((tetrahydro-2H-pyran-2-yl)oxy) acrylamide

1M Solution of boron tribromide solution in DCM (0.78 g, 3.11 mmol) was added dropwise to a reaction flask containing (E)-3-(4-(((1-(5-(3-(benzyloxy)-4-methoxyphenyl)-4-(4-cyano-3-fluorophenyl)thiophene-2-carbonyl)piperidin-4-yl)amino)methyl)phenyl)-N-((tetrahydro-2H-pyran-2-yl)oxy) acrylamide (60 mg, 0.075 mmol) and DCM (5 mL) at −60° C., and the mixture was stirred at −60° C. for 20 minutes. The reaction solution was quenched with water (10 mL), then extracted with ethyl acetate (10 mL×2). The organic phases were combined, washed with saturated brine (10 mL×2), dried over anhydrous sodium sulfate, and concentrated to afford (E)-3-(4-(((1-(4-(4-cyano-3-fluorophenyl)-5-(3-hydroxy-4-methoxyphenyl)thiophene-2-carbonyl)piperidin-4-yl)amino)methyl)phenyl)-N-((tetrahydro-2H-pyran-2-yl)oxy) acrylamide with a yield of 75%.

ESI-MS m/z=711.3 [M+H]⁺.

### Step j): preparation of (E)-3-(4-(((1-(4-(4-cyano-3-fluorophenyl)-5-(3-hydroxy-4-methoxyphenyl)thiophene-2-carbonyl)piperidin-4-yl)amino)methyl)phenyl)-N-hydroxyacrylamide hydrochloride

4.0M Hydrochloric acid solution in EA (4 mL) was added to a reaction flask containing (E)-3-(4-(((1-(4-(4-cyano-3-fluorophenyl)-5-(3-hydroxy-4-methoxyphenyl)thiophene-2-carbonyl)piperidin-4-yl)amino)methyl)phenyl)-N-((tetrahydro-2H-pyran-2-yl)oxy) acrylamide (40 mg, 0.056 mmol), and the mixture was stirred at room temperature for 1 hour. After concentration of the solution, the residue was purified by Prep-HPLC (separation method to 1) afford (E)-3-(4-(((1-(4-(4-cyano-3-fluorophenyl)-5-(3-hydroxy-4-methoxyphenyl)thiophene-2-carbonyl)piperidin-4-yl)amino)methyl)phenyl)-N-hydroxyacrylamide hydrochloride with a yield of 7.44%.

¹H NMR (400 MHz, DMSO-d₆) δ ppm 10.82 (s, 1H), 9.28 (d, J=1.6 Hz, 1H), 9.04 (s, 1H), 7.88 (t, J=7.6 Hz, 1H), 7.62 (s, 3H), 7.57-7.43 (m, 2H), 7.37-7.23 (m, 1H), 7.13 (d, J=12.6 Hz, 2H), 6.94 (d, J=8.4 Hz, 1H), 6.76-6.65 (m, 2H), 6.56-6.44 (m, 1H), 4.45 (d, J=13.2 Hz, 2H), 4.19 (s, 2H), 3.78 (s, 3H), 2.74 (d, J=2.4 Hz, 2H), 2.47 (s, 1H), 2.21 (d, J=12.4 Hz, 2H), 1.67 (d, J=12.8 Hz, 2H).

ESI-MS m/z=627.2 [M+H]⁺.

### Example 34

### Preparation of 7-(1-(7-(4-cyano-3-fluorophenyl)-8-(3-hydroxy-4-methylphenyl)imidazo[1,2-c]pyrimidin-5-yl)piperidin-4-yl)amino)-N-hydroxyheptanamide Hydrochloride, and the Starting Material were Prepared According to the Synthetic Method of Example 20

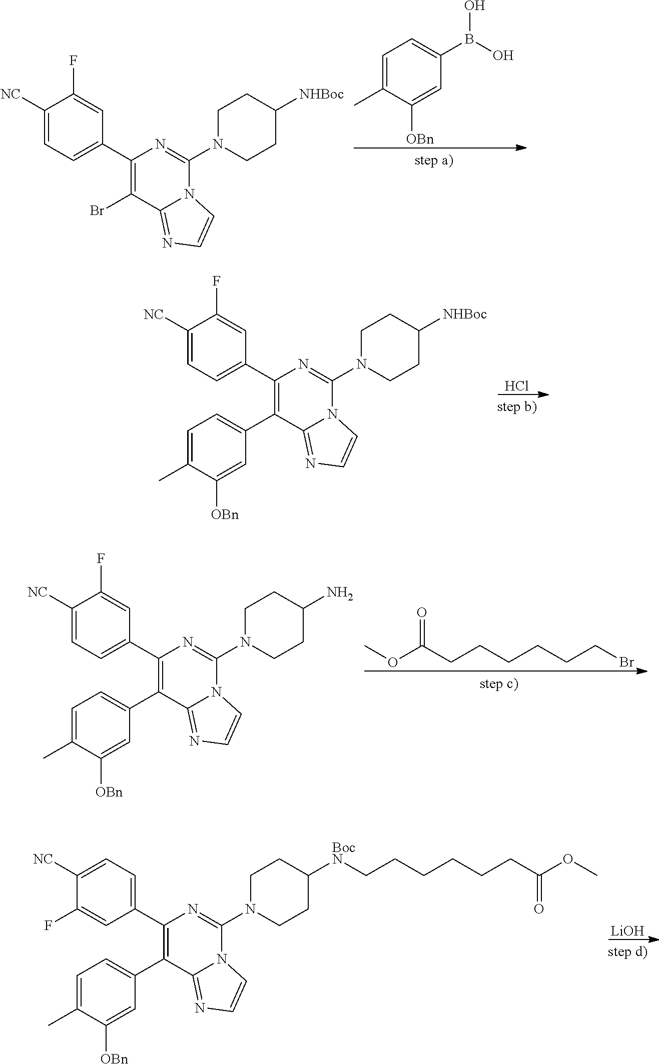

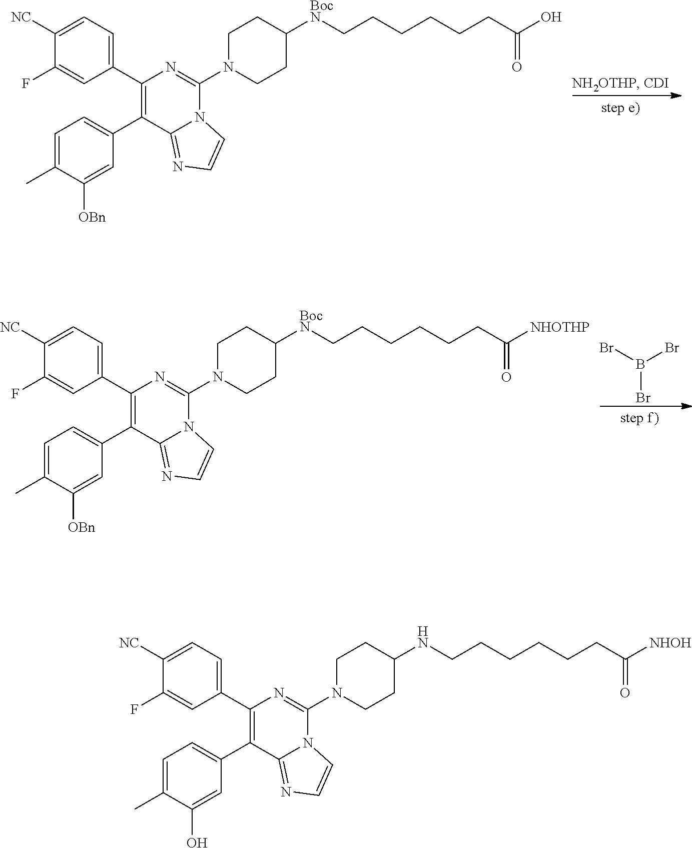

### Step a): preparation of tert-butyl (1-(8-(3-(benzyloxy)-4-methylphenyl)-7-(4-cyano-3-fluorophenyl)imidazo[1,2-c]pyrimidin-5-yl)piperidin-4-yl)carbamate

Tert-butyl (1-(8-bromo-7-(4-cyano-3-fluorophenyl)imidazo[1,2-c]pyrimidin-5-yl)piperidin-4-yl)carbamate (350 mg, 0.68 mmol), cesium carbonate (664.6 mg, 2.04 mmol), (3-(benzyloxy)-4-methylphenyl)boronic acid (246.9 mg, 1.02 mmol), and [1,1′-bis(diphenylphosphino)ferrocene]palladium dichloride (6.0 mg, 0.01 mmol) were dissolved in a mixture of 1,4-dioxane (12.5 mL) and water (2.5 mL), and the solution was subjected to microwave heating at 110° C. and reacted for 35 minutes under nitrogen protection. Water was added to dilute the mixture, which was then extracted with ethyl acetate (15 mL×3) The organic phases were combined and washed with saturated brine (10 mL×2), dried over anhydrous sodium sulfate, filtered, and concentrated under reduced pressure. The residue was purified by silica gel chromatography (eluent: ethyl acetate/petroleum ether=1:1) to afford tert-butyl (1-(8-(3-(benzyloxy)-4-methylphenyl)-7-(4-cyano-3-fluorophenyl)imidazo[1,2-c]pyrimidin-5-yl)piperidin-4-yl)carbamate with a yield of 84.9%.

ESI-MS m/z: 633.3 [M+H]⁺.

### Step b): preparation of 4-(5-(4-aminopiperidin-1-yl)-8-(3-(benzyloxy)-4-methylphenyl)imidazo[1,2-c]pyrimidin-7-yl)-2-fluorobenzonitrile

To the flask containing tert-butyl (1-(8-(3-(benzyloxy)-4-methylphenyl)-7-(4-cyano-3-fluorophenyl)imidazo[1,2-c]pyrimidin-5-yl)piperidin-4-yl)carbamate (230 mg, 0.36 mmol) was added hydrochloric acid solution in ethyl acetate (4M, 2 mL) and the mixture was stirred under nitrogen for 30 minutes. After the reaction was completed as indicated by LCMS, the solution was concentrated to afford 4-(5-(4-aminopiperidin-1-yl)-8-(3-(benzyloxy)-4-methylphenyl)imidazo[1,2-c]pyrimidin-7-yl)-2-fluorobenzonitrile with a yield of 95.1%.

ESI-MS m/z: 533.2 [M+H]⁺.

### Step c): preparation of 7-(1-(8-(3-(benzyloxy)-4-methylphenyl)-7-(4-cyano-3-methyl fluorophenyl)imidazo[1,2-c]pyrimidin-5-yl)piperidin-4-yl)(tert-butoxycarbonyl)amino)heptanoate

4-(5-(4-Aminopiperidin-1-yl)-8-(3-(benzyloxy)-4-methylphenyl)imidazo[1,2-c]pyrimidin-7-yl)-2-fluorobenzonitrile (182.2 mg, 0.34 mmol), methyl 7-bromoheptanoate (169.5 mg, 0.76 mmol), and potassium carbonate (234.9 mg, 1.7 mmol) were dissolved in dry DMSO (2 mL), and the mixture was stirred under nitrogen at 50° C. for 16 hours, then cooled to room temperature. Di-tert-butyl dicarbonate (83 mg, 0.38 mmol) was added, and the reaction was continued for an additional 30 minutes. The mixture was concentrated to dryness under reduced pressure, and the residue was purified by silica gel chromatography (eluent: ethyl acetate/petroleum ether=2:1) to afford methyl 7-(1-(8-(3-(benzyloxy)-4-methylphenyl)-7-(4-cyano-3-fluorophenyl)imidazo[1,2-c]pyrimidin-5-yl)piperidin-4-yl)(tert-butoxycarbonyl)amino)heptanoate with a yield of 17.8%.

ESI-MS m/z: 775.4 [M+H]⁺.

### Step d): preparation of 7-(1-(8-(3-(benzyloxy)-4-methylphenyl)-7-(4-cyano-3-fluorophenyl)imidazo[1,2-c]pyrimidin-5-yl)piperidin-4-yl)(tert-butoxycarbonyl)amino)heptanoic acid

Methyl 7-(1-(8-(3-(benzyloxy)-4-methylphenyl)-7-(4-cyano-3-fluorophenyl)imidazo[1,2-c]pyrimidin-5-yl)piperidin-4-yl)(tert-butoxycarbonyl)amino)heptanoate (80 mg, 0.10 mmol) was dissolved in a mixture of tetrahydrofuran:water=1:1 (2 mL), and lithium hydroxide (43.9 mg, 1.0 mmol) was added, and the mixture was stirred for 30 minutes, then extracted with ethyl acetate (10 mL×3). The organic phases were combined and washed with saturated brine (10 mL×2), dried over anhydrous sodium sulfate, filtered, and concentrated under reduced pressure to afford 7-(1-(8-(3-(benzyloxy)-4-methylphenyl)-7-(4-cyano-3-fluorophenyl)imidazo[1,2-c]pyrimidin-5-yl)piperidin-4-yl)(tert-butoxycarbonyl)amino)heptanoic acid with a yield of 94.6%.

ESI-MS m/z: 761.4 [M+H]⁺.

### Step e): preparation of tert-butyl (1-(8-(3-(benzyloxy)-4-methylphenyl)-7-(4-cyano-3-fluorophenyl)imidazo[1,2-c]pyrimidin-5-yl)piperidin-4-yl)(7-oxo-7-(((tetrahydro-2-pyranyl)carbamoyl)amino)heptane

7-(1-(8-(3-(Benzyloxy)-4-methylphenyl)-7-(4-cyano-3-fluorophenyl)imidazo[1,2-c]pyrimidin-5-yl)piperidin-4-yl)(tert-butoxycarbonyl)amino)heptanoic acid (90 mg, 0.12 mmol) and N,N′-carbonyldiimidazole (97.3 mg, 0.60 mmol) were dissolved in dry THF (3 mL), and the mixture was heated to 40° C. and stirred for 1 hour under nitrogen protection, then, triethylamine (121.4 mg, 1.2 mmol) was added, followed by adding 4-(aminooxy)tetrahydropyran (70.3 mg, 0.6 mmol), and the reaction was continued under nitrogen. After the reaction was completed as indicated by LCMS, the mixture was concentrated to dryness under reduced pressure, the residue was purified by silica gel chromatography (eluent: methanol/dichloromethane, 1:20) to afford tert-butyl (1-(8-(3-(benzyloxy)-4-methylphenyl)-7-(4-cyano-3-fluorophenyl)imidazo[1,2-c]pyrimidin-5-yl)piperidin-4-yl)(7-oxo-7-(((tetrahydro-2-pyranyl)carbamoyl)amino)heptane with a yield of 98.8%.

ESI-MS m/z: 860.4 [M+H]⁺.

### Step f): preparation of 7-(1-(7-(4-cyano-3-fluorophenyl)-8-(3-hydroxy-4-methylphenyl)imidazo[1,2-c]pyrimidin-5-yl)piperidin-4-ylamino)-N-hydroxyheptanamide hydrochloride

tert-butyl (1-(8-(3-(benzyloxy)-4-methylphenyl)-7-(4-cyano-3-fluorophenyl)imidazo[1,2-c]pyrimidin-5-yl)piperidin-4-yl)(7-oxo-7-(((tetrahydro-2-pyranyl)carbamoyl)amino)heptane (97 mg, 0.11 mmol) was dissolved in dry dichloromethane (1 mL). Under nitrogen thesolution was cooled in an ice bath to 0° C., and 1M boron tribromide (1.5 mL) was added, and the mixture was stirred for 30 minutes, resulting in the precipitation of a solid. Water (3 mL) was added to quench the reaction, and the mixture was filtered and concentrated. The residue was purified by Pre-HPLC (separation method 1) to afford 7-(1-(7-(4-cyano-3-fluorophenyl)-8-(3-hydroxy-4-methylphenyl)imidazo[1,2-c]pyrimidin-5-yl)piperidin-4-ylamino)-N-hydroxyheptanamide hydrochloride with a yield of 22.9%.

¹H NMR (400 MHZ, DMSO-d₆) δ ppm 10.39 (s, 1H), 9.71 (d, J=16.2 Hz, 1H), 9.10 (s, 2H), 8.23 (s, 1H), 8.08 (s, 1H), 7.88 (dd, J=8.2, 6.9 Hz, 1H), 7.59 (dd, J=10.6, 1.5 Hz, 1H), 7.35 (dd, J=8.2, 1.6 Hz, 1H), 7.17 (d, J=7.6 Hz, 1H), 6.80 (d, J=1.8 Hz, 1H), 6.64 (dd, J=7.6, 1.8 Hz, 1H), 4.14 (s, 2H), 3.40 (s, 1H), 3.23 (t, J=12.6 Hz, 2H), 2.92 (s, 2H), 2.18 (s, 5H), 1.94 (dt, J=12.0, 5.9 Hz, 4H), 1.66 (p, J=7.6 Hz, 2H), 1.50 (p, J=7.4 Hz, 2H), 1.30 (dq, J=12.6, 8.0, 7.0 Hz, 4H).

ESI-MS m/z: 586.3 [M+H]⁺.

Examples 35-36 were prepared similarly according to the synthetic method of Example 34 (the separation method for the compounds: hydrochloride and formate were prepared according to separation method 1 and 3, respectively), and the structure and characterization data are as follows:

| Ex- | | | | MS |
|---|---|---|---|---|
| am- | | | | (M + |
| ple | Chemical name | Structure | ¹H NMR | H)⁺ |
| 35 | 7-((1-(7-(4-Cyano-3- fluorophenyl)-8-(3- hydroxy-4-methoxy- phenyl)imidazo [1,2-c]pyrimidin-5- yl)piperidin-4- yl)amino)-N- hydroxy- heptanamide hydrochloride | 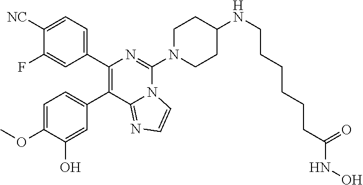 [SMILES: COc1ccc(-c2c(-c3ccc(C#N)c(F)c3)nc(N3CCC(NCCCCCCC(=O)NO)CC3)n3ccnc23)cc1O] | ¹H NMR (400 MHz, Methanol-d₄) δ 8.22 (s, 1H), 8.01 (s, 1H), 7.68 (t, J = 7.2 Hz, 1H), 7.54 (d, J = 10.4 Hz, 1H), 7.45 (d, J = 8.0 Hz, 1H), 7.08 (d, J = 7.4 Hz, 1H), 6.83 (d, J = 11.0 Hz, 2H), 4.29 (d, J = 13.0 Hz, 2H), 3.93 (s, 3H), 3.55 (s, 1H), 3.39 (t, J = 12.0 Hz, 2H), 3.11 (s, 2H), 2.40-2.26 (m, 2H), 2.19 (d, J = 6.8 Hz, 2H), 2.17-1.99 (m, 2H), 1.84-1.59 (m, 4H), 1.46 (d, J = 13.8 Hz, 4H). | 602.3 |
| 36 | (E)-3-(4-(((1-(7-(4- Cyano-3- fluorophenyl)-8-(5- fluoro-3-methyl- benzo[d]isoxazol- 6-yl)imidazo[1,2- c]pyrimidin-5- yl)piperidin-4- yl)amino)methyl) phenyl)-N- hydroxyacrylamide formate | 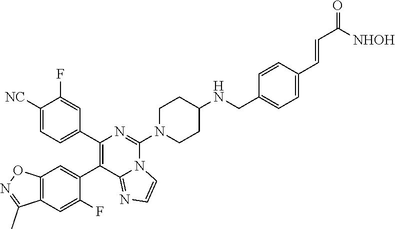 | ¹H NMR (400 MHz, DMSO- d₆) δ ppm: 10.76 (s, 1H), 8.38 (s, 1H), 7.93 (d, J = 1.6 Hz, 1H), 7.88 (d, J = 5.4 Hz, 1H), 7.79 (dd, J = 8.4, 6.4 Hz, 2H), 7.65 (d, J = 1.4 Hz, 1H), 7.58 (dd, J = 10.8, 1.6 Hz, 1H), 7.52 (d, J = 8.0 Hz, 2H), 7.45-7.39 (m, 3H), 7.28 (dd, J = 8.2, 1.6 Hz, 1H), 4.05- 3.94 (m, 2H), 3.81 (s, 2H), 3.16 (t, J = 11.8 Hz, 2H), 2.75 (dt, J = 9.8, 5.4 Hz, 1H), 2.57 (s, 3H), 2.04 (d, J = 12.6 Hz, 2H), 1.61 (d, J = 12.0 Hz, 2H). | 661.2 |

### Example 37

### Preparation of (E)-3-(4-(((1-(3-cyano-4-(4-cyano-3-fluorophenyl)-5-(3-hydroxy-4-methoxyphenyl)pyridin-2-yl)piperidin-4-yl)amino)methyl)phenyl)-N-hydroxyacrylamide hydrochloride

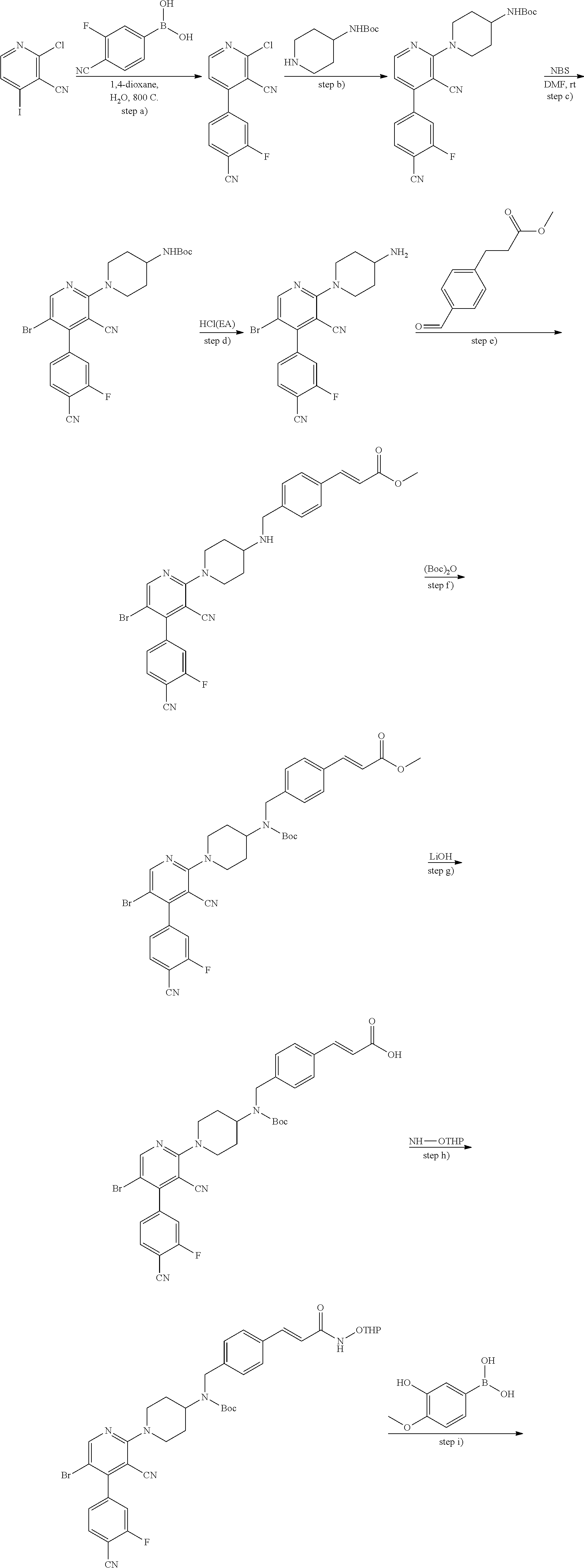

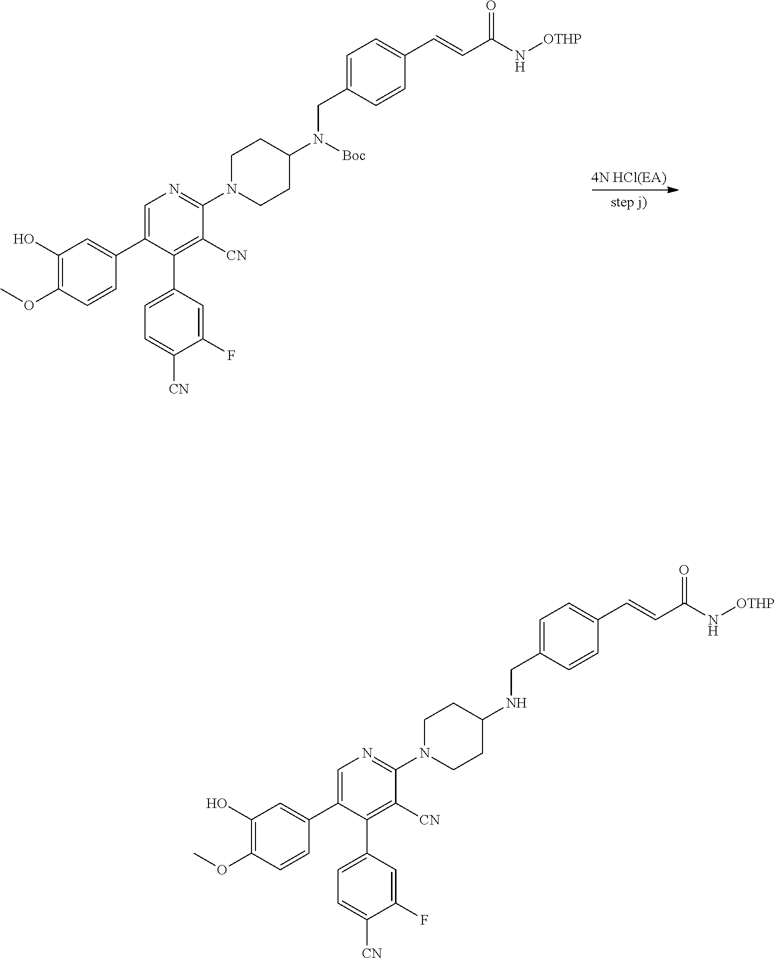

### Step a): preparation of 2-chloro-4-(4-cyano-3-fluorophenyl)nicotinonitrile

2-Chloro-4-iodopyridine-3-carbonitrile (6.4 g, 24.20 mmol), (4-cyanophenyl)boronic acid (4.19 g, 25.41 mmol), [1,1′-bis(diphenylphosphino)ferrocene]dichloropalladium (II) (1.77 g, 2.42 mmol), and cesium carbonate (14.19 g, 43.56 mmol) were added to a mixture of 1,4-dioxane: H₂O=5:1 (80 mL). After nitrogen purging, the solution was heated to 100° C. for 1 hour, and concentrated to remove the solvent. The residue was mixed with silica gel and purified a normal-phase column to afford 2-chloro-4-(4-cyano-3-fluorophenyl)pyridine-3-carbonitrile with a yield of 93.02%.

ESI-MS m/z: 258.1 [M+H]⁺.

### Step b): preparation of tert-butyl N-(1-(3-cyano-4-(4-cyano-3-fluorophenyl)pyridin-2-yl)piperidin-4-yl)carbamate

2-Chloro-4-(4-cyano-3-fluorophenyl)pyridine-3-carbonitrile (2.0 g, 7.76 mmol), tert-butyl piperidin-4-ylcarbamate (1.6 g, 8.15 mmol), and ethyl bis(2-propyl)amine (1.2 g, 9.31 mmol) were added to NMP (100 mL), and the mixture was heated to 120° C. and reacted overnight. The reaction was monitored by LCMS until the starting materials were completely consumed, the solution was then extracted after adding water with ethyl acetate. The organic phases were combined, washed with saturated brine, and concentrated. The residue was mixed with silica gel and purified by a normal-phase column to afford tert-butyl N-(1-(3-cyano-4-(4-cyano-3-fluorophenyl)pyridin-2-yl)piperidin-4-yl)carbamate with a yield of 85.6%.

ESI-MS m/z: 422.2 [M+H]⁺.

### Step c): preparation of tert-butyl N-(1-(5-bromo-3-cyano-4-(4-cyano-3-fluorophenyl)pyridin-2-yl)piperidin-4-yl)carbamate

Tert-butyl (1-(3-cyano-4-(4-cyano-3-fluorophenyl)pyridin-2-yl)piperidin-4-yl)carbamate (2.0 g, 4.7 mmol) and N-bromosuccinimide (3.3 g, 18.5 mmol) were added to DMF (4 mL), and the mixture was stirred at room temperature for 1 hour, and the reaction was monitored by LCMS until the starting materials were consumed. Water was then added, and the mixture was extracted with ethyl acetate. The organic phases were combined, and concentrated to remove the solvent. The residue was mixed with silica gel and purified by a normal-phase column to afford tert-butyl N-(1-(5-bromo-3-cyano-4-(4-cyano-3-fluorophenyl)pyridin-2-yl)piperidin-4-yl)carbamate with a yield of 93.5%. ESI-MS m/z: 500.1 [M+H]⁺.

### Step d): preparation of 2-(4-Aminopiperidin-1-yl)-5-bromo-4-(4-cyano-3-fluorophenyl)pyridine-3-formonitrile

Tert-butyl N-(1-(5-bromo-3-cyano-4-(4-cyano-3-fluorophenyl)pyridin-2-yl)piperidin-4-yl)carbamate (1.0 g, 2.0 mmol) was added to 4N HCl/EA (10 mL) and the mixture was reacted at room temperature for 0.5 hours, and the reaction was monitored by LCMS until the starting materials were consumed. The reaction solution was poured into a saturated sodium bicarbonate aqueous solution, then extracted with ethyl acetate, and the organic phase was washed with saturated brine. After removing the solvent, the crude product 2-(4-aminopiperidin-1-yl)-5-bromo-4-(4-cyano-3-fluorophenyl)pyridine-3-formonitrile was obtained with a yield of 91.2%.

ESI-MS m/z: 400.1 [M+H]⁺.

### Step e): preparation of methyl (E)-3-(4-((1-(5-bromo-3-cyano-4-(4-cyano-3-fluorophenyl)pyridin-2-yl)piperidin-4-yl)amino)methyl)phenyl)acrylate

2-(4-Aminopiperidin-1-yl)-5-bromo-4-(4-cyano-3-fluorophenyl)pyridine-3-formonitrile (600 mg, 1.50 mmol) and methyl (E)-3-(4-formylphenyl)acrylate (381.55 mg, 1.50 mmol) were added to a mixture of DCE:MeOH:AcOH=10:0.1:0.01 (20 mL), and the mixture was stirred at room temperature for 0.5 hour, then cooled in an ice bath, and sodium cyanoborohydride (285.26 mg, 4.5 mmol) was added. The reaction was maintained in the ice bath for 2 hours and monitored by LCMS. After completion, the reaction mixture was quenched by the addition of aqueous sodium bicarbonate and it was then extracted with DCM. The combined organic solution was dried over sodium sulfate and concentrated under reduced pressure. The resultant crude product was then used directly for the next step without further purification.

ESI-MS m/z: 574.1 [M+H]⁺.

### Step f): preparation of f methyl (E)-3-(4-((1-(5-bromo-3-cyano-4-(4-cyano-3-fluorophenyl)pyridin-2-yl)piperidin-4-yl)(tert-butoxycarbonyl)amino)methyl)phenyl)acrylate

Methyl (E)-3-(4-((1-(5-bromo-3-cyano-4-(4-cyano-3-fluorophenyl)pyridin-2-yl)piperidin-4-yl)amino)methyl)phenyl)acrylate (200 mg, 0.35 mmol) was dissolved in a mixture of THF/H₂O=5:1 (5 mL), and di-tert-butyl dicarbonate (78 mg, 0.35 mmol) was added. The reaction was stirred at room temperature for 1 hour and the reaction progress was monitored by LCMS. Upon completion, the reaction mixture was extracted with EA, and the organic phase was separated and washed with saturated brine. The volatile components were then removed under reduced pressure, the crude product such obtained was then purified by column chromatography on silica gel to afford methyl (E)-3-(4-(((1-(5-bromo-3-cyano-4-(4-cyano-3-fluorophenyl)pyridin-2-yl)piperidin-4-yl)(tert-butoxycarbonyl)amino)methyl)phenyl)acrylate with a yield of 84.71%.

ESI-MS m/z: 674.2 [M+H]⁺.

### Step g): preparation of (E)-3-(4-((1-(5-bromo-3-cyano-4-(4-cyano-3-fluorophenyl)pyridin-2-yl)piperidin-4-yl)(tert-butoxycarbonyl)amino)methyl)phenyl)acrylic acid

methyl (E)-3-(4-(((1-(5-bromo-3-cyano-4-(4-cyano-3-fluorophenyl)pyridin-2-yl)piperidin-4-yl)(tert-butoxycarbonyl)amino)methyl)phenyl)acrylate (100 mg, 0.15 mmol) and lithium hydroxide (17.96 mg, 0.75 mmol) were added to a mixture of THF/H₂O=5:1 (1 mL), and was stirred at room temperature overnight. The reaction was monitored by LCMS until the starting materials were consumed. The pH was adjusted to weakly acidic with dilute hydrochloric acid, and EA was then added for extraction. The organic phase was washed with saturated brine, and the solvent was removed under reduced pressure to obtain a solid product, which was used directly in the next step without further purification.

ESI-MS m/z: 660.2 [M+H]⁺.

### Step h): preparation of tert-butyl (E)-(1-(5-bromo-3-cyano-4-(4-cyano-3-fluorophenyl)pyridin-2-yl)piperidin-4-yl)(4-(3-oxo-3-((tetrahydro-2H-pyran-2-yl)oxy)amino)prop-1-en-1-yl)benzyl)carbamate

The crude material obtained from Step g above (100 mg, 0.15 mmol), O-(tetrahydro-2H-pyran-2-yl)hydroxylamine (21.09 mg, 0.18 mmol), HATU (68.44 mg, 0.18 mmol), and ethyldiisopropylamine (58.16 mg, 0.45 mmol) were added to DMF (4 mL). The reaction was stirred at room temperature for 0.5 hours and monitored by LCMS until the starting materials were consumed. The solution was poured into water, and EA was added for extraction. The organic phase was concentrated under reduced pressure, the resultant residue was mixed with silica gel and purified by a normal-phase column to afford the product tert-butyl (E)-(1-(5-bromo-3-cyano-4-(4-cyano-3-fluorophenyl)pyridin-2-yl)piperidin-4-yl)(4-(3-oxo-3-((tetrahydro-2H-pyran-2-yl)oxy)amino)prop-1-en-1-yl)benzyl)carbamate with a yield of 71.96%.

ESI-MS m/z: 759.2 [M+H]⁺.

### Step i): preparation of tert-butyl (E)-(1-(3-cyano-4-(4-cyano-3-fluorophenyl)-5-(3-hydroxy-4-methoxyphenyl)pyridin-2-yl)piperidin-4-yl)(4-(3-oxo-3-(((tetrahydro-2H-pyran-2-yl)oxy)amino)prop-1-en-1-yl)benzyl)carbamat.e

Tert-butyl (E)-(1-(5-bromo-3-cyano-4-(4-cyano-3-fluorophenyl)pyridin-2-yl)piperidin-4-yl)(4-(3-oxo-3-((tetrahydro-2H-pyran-2-yl)oxy)amino)prop-1-en-1-yl)benzyl)carbamate (100 mg, 0.13 mmol), (3-hydroxy-4-methoxyphenyl)boronic acid (34 mg, 0.20 mmol), [1,1′-bis(diphenylphosphino)ferrocene]palladium (II) dichloride (9.51 mg, 0.013 mmol), and cesium carbonate (84.71 mg, 0.26 mmol) were added to a mixture of dioxane/H₂O=5:1 (5 mL). After purging with nitrogen, the mixture was subjected to microwave heating at 110° C. for 1 hour, and the reaction was monitored by LCMS until the starting materials were consumed. The solvent was removed and the residue was mixed with silica gel and purified by a normal-phase column to afford tert-butyl (E)-(1-(3-cyano-4-(4-cyano-3-fluorophenyl)-5-(3-hydroxy-4-methoxyphenyl)pyridin-2-yl)piperidin-4-yl)(4-(3-oxo-3-(((tetrahydro-2H-pyran-2-yl)oxy)amino)prop-1-en-1-yl)benzyl)carbamate with a yield of 82.39%.

ESI-MS m/z: 719.3 [M+H]⁺.

### Step j): preparation of (E)-3-(4-(((1-(3-cyano-4-(4-cyano-3-fluorophenyl)-5-(3-hydroxy-4-methoxyphenyl)pyridin-2-yl)piperidin-4-yl)amino)methyl)phenyl)-N-hydroxyacrylamide hydrochloride

tert-butyl (E)-(1-(3-cyano-4-(4-cyano-3-fluorophenyl)-5-(3-hydroxy-4-methoxyphenyl)pyridin-2-yl)piperidin-4-yl)(4-(3-oxo-3-(((tetrahydro-2H-pyran-2-yl)oxy)amino)prop-1-en-1-yl)benzyl)carbamate (60 mg, 75 μmol) was added to 4N HCl (EA) (4 mL), and the solution was stirred at room temperature for 0.5 hours and monitored by LCMS until the starting materials were consumed. The solvent was removed, and the product was purified by Prep-HPLC (separation method 1) to afford (E)-3-(4-(((1-(3-cyano-4-(4-cyano-3-fluorophenyl)-5-(3-hydroxy-4-methoxyphenyl)pyridin-2-yl)piperidin-4-yl)amino)methyl)phenyl)-N-hydroxyacrylamide hydrochloride with a yield of 71.12%.

¹H NMR (400 MHZ, DMSO-d₆) δ 10.41 (d, J=18.6 Hz, 1H), 8.39 (s, 3H), 7.86 (t, J=7.6 Hz, 1H), 7.50 (d, J=10.4 Hz, 1H), 7.29 (dd, J=8.0, 1.4 Hz, 1H), 6.84 (d, J=8.0 Hz, 1H), 6.70 (s, 1H), 6.59 (d, J=2.2 Hz, 1H), 6.45 (dd, J=8.2, 2.0 Hz, 1H), 4.47 (d, J=13.2 Hz, 2H), 4.17 (t, J=6.2 Hz, 2H), 3.79 (s, 3H), 3.39 (dq, J=11.4, 5.6 Hz, 1H), 3.13 (t, J=12.8 Hz, 2H), 2.13-2.04 (m, 2H), 1.98 (t, J=7.4 Hz, 2H), 1.68 (tq, J=13.6, 6.8, 5.2 Hz, 4H), 1.51 (p, J=7.4 Hz, 2H), 1.30 (dq, J=16.8, 6.6, 5.4 Hz, 4H).

ESI-MS m/z: 619.2 [M+H]⁺.

Examples 39-53 were prepared similarly according to the synthetic method of Example 37 (the separation method for the compounds: hydrochloride and formate were prepared by separation method 1 and 3, respectively), and the structure and characterization data are as follows:

| Ex- | | | | MS |
|---|---|---|---|---|
| am- | | | | (M + |
| ple | Chemical name | Structure | ¹H NMR | H)⁺ |
| 39 | 4-((4-(4-(5-Cyano-4- (4-cyano-3-fluoro- phenyl)-6-(4- (methylamino) piperidin- 1-yl)pyridin-3-yl)-2- hydroxyphenyl) amino)methyl)-N- hydroxybenzamide hydrochloride | 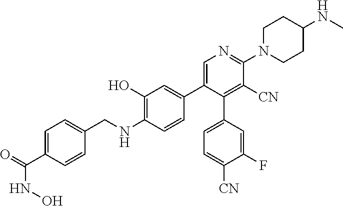 [SMILES: CNC1CCN(c2ncc(-c3ccc(NCc4ccc(C(=O)NO)cc4)c(O)c3)c(-c3ccc(C#N)c(F)c3)c2C#N)CC1] | ¹H NMR (400 MHz, Methanol- d₄) δ 8.44 (s, 1H), 7.88-7.69 (m, 3H), 7.54-7.33 (m, 3H), 7.27 (dd, J = 8.0, 1.3 Hz, 1H), 7.12 (d, J = 8.1 Hz, 1H), 6.74 (d, J = 1.6 Hz, 1H), 6.67 (dd, J = 8.1, 1.8 Hz, 1H), 4.61-4.43 (m, 4H), 3.40 (tt, J = 11.5, 4.0 Hz, 1H), 3.25-3.16 (m, 2H), 2.76 (s, 3H), 2.36-2.18 (m, 2H), 1.80 (qd, J = 12.2, 3.9 Hz, 2H). | 592.3 |
| 40 | (E)-3-(4-(((1-(3- Cyano-4-(4-cyano-3- fluorophenyl)-5-(3- fluoro-4- methoxyphenyl) pyridin- 2-yl)piperidin-4- yl)amino)methyl) phenyl)-N- hydroxyacrylamide hydrochloride | 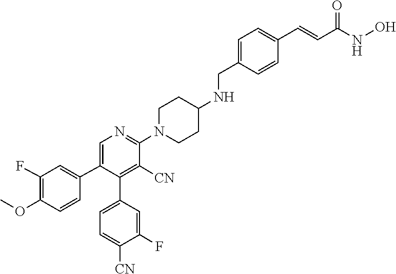 [SMILES: COc1ccc(-c2cnc(N3CCC(NCc4ccc(/C=C/C(=O)NO)cc4)CC3)c(C#N)c2-c2ccc(C#N)c(F)c2)cc1F] | ¹H NMR (400 MHz, DMSO-d₆) δ 8.48 (s, 3H), 8.46 (s, 1H), 7.97 (dd, J = 8.0, 6.8 Hz, 1H), 7.67 (dd, J = 10.0, 1.6 Hz, 1H), 7.50 (d, J = 8.0 Hz, 2H), 7.41 (t, J = 8.8 Hz, 2H), 7.37-7.27 (m, 2H), 7.10-6.94 (m, 2H), 6.79 (dt, J = 8.4, 1.6 Hz, 1H), 6.45 (d, J = 15.8 Hz, 1H), 4.19 (d, J = 13.0 Hz, 2H), 3.79 (m, 5H), 3.19 (t, J = 11.8 Hz, 2H), 2.77-2.63 (m, 1H), 2.03-1.88 (m, 2H), 1.43 (q, J = 11.2, 10.8 Hz, 2H). | 621.2 |
| 41 | 4-(((1-(3- Cyano-4-(4-cyano- 3-fluorophenyl)- 5-(3-hydroxy-4- methoxyphenyl) pyridin- 2-yl)piperidin-4- yl)amino)methyl)-N- hydroxybenzamide hydrochloride | 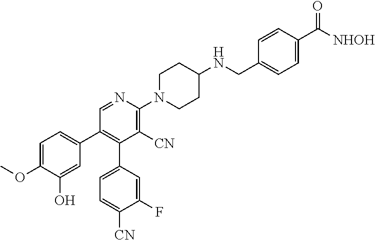 | ¹H NMR (400 MHz, Methanol- d₄) δ 8.50 (s, 1H), 8.42 (s, 1H), 7.80 (d, J = 7.6 Hz, 2H), 7.74 (t, J = 7.2 Hz, 1H), 7.56 (d, J = 7.8 Hz, 2H), 7.36 (d, J = 9.6 Hz, 1H), 7.23 (d, J = 7.8 Hz, 1H), 6.81 (d, J = 8.6 Hz, 1H), 6.50 (d, J = 6.6 Hz, 2H), 4.41 (d, J = 13.2 Hz, 2H), 4.15 (s, 2H), 3.81 (s, 3H), 3.15 (t, J = 12.6 Hz, 3H), 2.22 (d, J = 12.2 Hz, 2H), 1.74 (d, J = 12.6 Hz, 2H). | 593.2 |
| 42 | (E)-3-(4-(((1-(3- Cyano-4-(4-cyano-3- fluorophenyl)-5-(3- hydroxy-4-methoxy- phenyl)pyridin- 2-yl)piperidin-4- yl)(methyl)amino) methyl)phenyl)-N- hydroxyacrylamide formate | 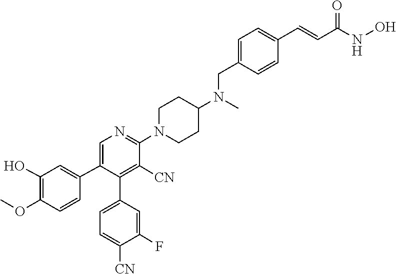 [SMILES: COc1ccc(-c2cnc(N3CCC(N(C)Cc4ccc(/C=C/C(=O)NO)cc4)CC3)c(C#N)c2-c2ccc(C#N)c(F)c2)cc1O] | ¹H NMR (400 MHz, DMSO-d₆) δ 10.73 (s, 1H), 9.05 (s, 1H), 8.42 (s, 1H), 7.95 (dd, J = 8.0, 6.8 Hz, 1H), 7.66 (dd, J = 10.2, 1.6 Hz, 1H), 7.51 (d, J = 7.8 Hz, 2H), 7.44 (d, J = 15.8 Hz, 1H), 7.36 (d, J = 7.8 Hz, 2H), 7.28 (dd, J = 8.0, 1.6 Hz, 1H), 6.80 (d, J = 8.0 Hz, 1H), 6.52-6.39 (m, 3H), 4.34 (d, J = 12.8 Hz, 2H), 3.71 (s, 3H), 3.61 (s, 2H), 3.07 (t, J = 12.4 Hz, 2H), 2.80-2.62 (m, 1H), 2.14 (s, 3H), 1.93 (d, J = 12.2 Hz, 2H), 1.77-1.54 (m, 2H). | 633.3 |
| 43 | 3-(4-((1-(3- Cyano-4-(4-cyano- 3-fluorophenyl)- 5-(3-hydroxy-4- methoxyphenyl) pyridin-2-yl) piperidin-4- yl)amino)methyl) phenyl)-N- hydroxypropanamide formate | 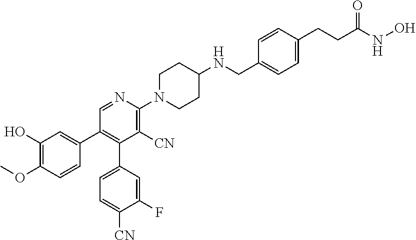 [SMILES: COc1ccc(-c2cnc(N3CCC(NCc4ccc(CCC(=O)NO)cc4)CC3)c(C#N)c2-c2ccc(C#N)c(F)c2)cc1O] | ¹H NMR (400 MHz, DMSO-d₆) δ 8.48 (s, 2H), 8.46 (s, 1H), 7.97 (dd, J = 8.0, 6.8 Hz, 1H), 7.67 (dd, J = 10.0, 1.6 Hz, 1H), 7.50 (d, J = 8.0 Hz, 2H), 7.41 (t, J = 8.8 Hz, 2H), 7.37-7.27 (m, 2H), 7.10-6.94 (m, 2H), 6.79 (dt, J = 8.4, 1.6 Hz, 1H), 6.45 (d, J = 15.8 Hz, 1H), 4.19 (d, J = 13.0 Hz, 2H), 3.79 (m, 5H), 3.19 (t, J = 11.8 Hz, 2H), 2.77-2.63 (m, 3H), 2.28 (t, J = 12.0, 2H) 2.03- 1.88 (m, 2H), 1.43 (q, J = 11.2, 10.8 Hz, 2H). | 621.3 |
| 44 | (E)-3-(4-(((1-(3- Cyano-4-(4-cyano-3- fluorophenyl)-5-(3- hydroxy-4- methoxyphenyl) pyridin-2-yl)- 4-methylpiperidin- 4-yl)amino) methyl)phenyl)- N-hydroxyacrylamide formate | 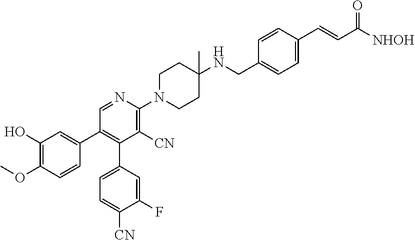 | ¹H NMR (400 MHz, DMSO-d₆) δ ppm: 8.44 (s, 2H), 8.40 (s, 1H), 7.95 (dd, J = 8.0, 6.8 Hz, 1H), 7.66 (dd, J = 10.2, 1.6 Hz, 1H), 7.47-7.39 (m, 5H), 7.27 (dd, J = 8.0, 1.6 Hz, 1H), 6.83-6.77 (m, 1H), 6.50-6.44 (m, 2H), 3.79 (d, J = 13.6 Hz, 2H), 3.71 (m, 5H), 3.67 (d, J = 10.2 Hz, 2H), 1.81-1.71 (m, 2H), 1.64- 1.52 (m, 2H), 1.18 (s, 3H). | 633.3 |
| 45 | (E)-3-(4-(2-((1-(3- Cyano-4-(4-cyano-3- fluorophenyl)-5-(3- hydroxy-4- methoxyphenyl) pyridin- 2-yl)piperidin-4- yl)amino)ethyl) phenyl)-N- hydroxyacrylamide hydrochloride | 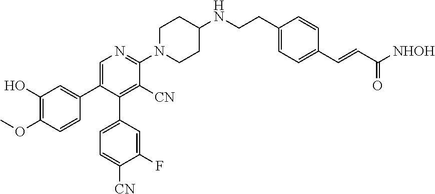 | ¹H NMR (400 MHz, Methanol- d₄) δ 8.44 (s, 1H), 7.74 (dd, J = 8.0, 6.6 Hz, 1H), 7.63-7.48 (m, 3H), 7.37 (dd, J = 8.0, 1.8 Hz, 3H), 7.23 (dd, J = 8.0, 1.6 Hz, 1H), 6.90-6.75 (m, 1H), 6.59- 6.36 (m, 3H), 4.43 (d, J = 13.6 Hz, 2H), 3.5 (s, 1H), 3.81 (s, 3H), 3.37 (d, J = 8.2 Hz, 2H), 3.17 (t, J = 12.0 Hz, 2H), 3.10-2.98 (m, 2H), 2.27 (d, J = 10.8 Hz, 2H), 1.91-1.73 (m, 2H). | 633.3 |
| 46 | 5-(1-(3-Cyano-4-(4- cyano-3- fluorophenyl)- 5-(3-hydroxy-4- methoxyphenyl) pyridin- 2-yl)piperidin-4- yl)amino)-N- hydroxypentanamide formate | 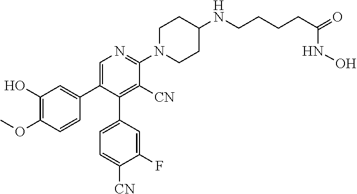 [SMILES: COc1ccc(-c2cnc(N3CCC(NCCCCC(=O)NO)CC3)c(C#N)c2-c2ccc(C#N)c(F)c2)cc1O] | ¹H NMR (400 MHz, DMSO-d₆) δ ppm: 8.42 (s, 1H), 8.26 (s, 1H), 7.95 (dd, J = 8.0, 6.8 Hz, 1H), 7.66 (dd, J = 10.2, 1.4 Hz, 1H), 7.27 (dd, J = 8.0, 1.6 Hz, 1H), 6.84-6.76 (m, 1H), 6.47 (d, J = 7.2 Hz, 2H), 4.21 (d, J = 13.0 Hz, 2H), 3.71 (s, 3H), 3.22-3.08 (m, 3H), 2.70 (t, J = 7.2 Hz, 2H), 2.07-1.93 (m, 4H), 1.62-1.40 (m, 6H). | 559.3 |
| 47 | 1-(3-Cyano- 4-(4-cyano-3- fluorophenyl)-5-(3- hydroxy-4- methoxyphenyl) pyridin- 2-yl)piperidin-4- ylamino)-N- hydroxyheptanamide formate | 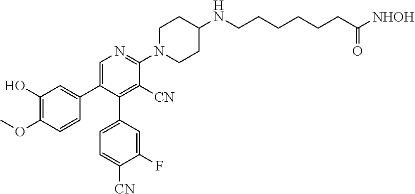 | ¹H NMR (400 MHz, DMSO-d₆) δ ppm: 10.35 (s, 1H), 9.05 (s, 1H), 8.43 (s, 1H), 8.29 (s, 1H), 7.96 (dd, J = 8.0, 6.8 Hz, 1H), 7.67 (dd, J = 10.2, 1.6 Hz, 1H), 7.28 (dd, J = 8.0, 1.6 Hz, 1H), 6.84-6.75 (m, 1H), 6.52-6.42 (m, 2H), 4.22 (d, J = 13.2 Hz, 2H), 3.71 (s, 3H), 3.14 (t, J = 12.2 Hz, 2H), 2.96 (s, 1H), 2.70 (q, J = 8.8, 8.2 Hz, 2H), 2.04 (d, J = 12.2 Hz, 2H), 1.94 (t, J = 7.4 Hz, 2H), 1.49 (p, J = 7.6 Hz, 6H), 1.28 (tt, J = 12.4, 6.8 Hz, 4H). | 587.3 |
| 48 | 2-(1-(3-Cyano-4-(4- cyano-3- fluorophenyl)- 5-(3-hydroxy-4- methoxyphenyl) pyridin-2-yl) piperidin-4-yl)-N- hydroxyacetamide formate | 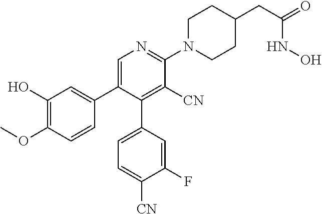 [SMILES: COc1ccc(-c2cnc(N3CCC(CC(=O)NO)CC3)c(C#N)c2-c2ccc(C#N)c(F)c2)cc1O] | ¹H NMR (400 MHz, Methanol- d₄) δ ppm 8.55 (s, 1H), 8.38 (s, 1H), 7.73 (dd, J = 8.0, 6.6 Hz, 1H), 7.35 (dd, J = 9.8, 1.6 Hz, 1H), 7.22 (dd, J = 8.0, 1.6 Hz, 1H), 6.88-6.77 (m, 1H), 6.50 (d, J = 6.8 Hz, 2H), 4.33 (d, J = 13.0 Hz, 2H), 3.80 (s, 3H), 3.21-2.93 (m, 2H), 2.16-2.02 (m, 3H), 1.94-1.76 (m, 2H), 1.43 (q, J = 11.6, 11.2 Hz, 2H). | 502.2 |
| 49 | (E)-3-(4-(2-(4-(3- Cyano-4-(4-cyano-3- fluorophenyl)-5-(3- hydroxy-4-methoxy- phenyl)pyridin- 2-yl)piperazin-1- yl)ethyl)phenyl)-N- hydroxyacrylamide hydrochloride | 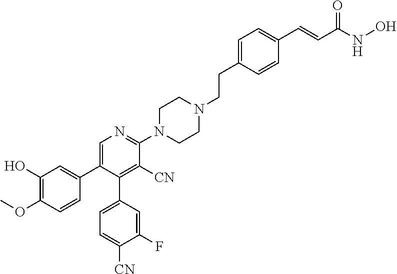 [SMILES: COc1ccc(-c2cnc(N3CCN(CCc4ccc(/C=C/C(=O)NO)cc4)CC3)c(C#N)c2-c2ccc(C#N)c(F)c2)cc1O] | ¹H NMR (400 MHz, DMSO-d₆) δ ppm 11.04 (s, 1H), 10.77 (s, 1H), 9.07 (s, 1H), 8.53 (s, 1H), 7.99 (t, J = 7.2 Hz, 1H), 7.66 (d, J = 10.0 Hz, 1H), 7.55 (d, J = 8.0 Hz, 2H), 7.44 (d, J = 8.0 Hz, 1H), 7.34 (d, J = 8.0 Hz, 2H), 7.28 (d, J = 8.0 Hz, 1H), 6.83 (d, J = 8.0 Hz, 1H), 6.50-6.44 (m, 3H), 4.31 (d, J = 13.6 Hz, 2H), 3.78-3.72 (m, 5H), 3.62-3.56 (m, 4H), 3.27-3.22 (m, 2H), 3.15-3.11 (m, 2H). | 619.3 |
| 50 | (E)-3-(4-(((1-(3- Cyano-4-(4-cyano-3- fluorophenyl)-5-(3- hydroxy-4- methoxyphenyl)-6- methylpyridin-2- yl)piperidin-4- yl)amino)methyl) phenyl)-N- hydroxyacrylamide- formate | 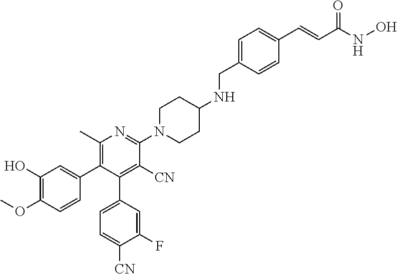 [SMILES: COc1ccc(-c2c(C)nc(N3CCC(NCc4ccc(/C=C/C(=O)NO)cc4)CC3)c(C#N)c2-c2ccc(C#N)c(F)c2)cc1O] | ¹H NMR (400 MHz, Methanol- d₄) δ 8.53 (m, 4H), 7.63 (dd, J = 16.8, 7.6 Hz, 3H), 7.52 (d, J = 15.6 Hz, 2H), 7.25 (d, J = 9.6 Hz, 1H), 7.16 (d, J = 7.8 Hz, 1H), 6.80 (d, J = 8.0 Hz, 1H), 6.50- 6.41 (m, 2H), 4.42 (d, J = 13.0 Hz, 2H), 4.09 (s, 2H), 3.80 (s, 3H), 3.12 (dd, J = 15.8, 9.0 Hz, 3H), 2.31 (s, 3H), 2.19 (t, J = 7.8 Hz, 2H), 1.72 (d, J = 12.8 Hz, 2H). | 633.3 |
| 51 | (E)-4-((1-(3- Cyano-4-(4-cyano- 3-fluorophenyl)-5- (3-hydroxy-4- methoxyphenyl) pyridin- 2-yl)piperidin-4- yl)amino)-N- hydroxybut- 2-enamide hydrochloride | 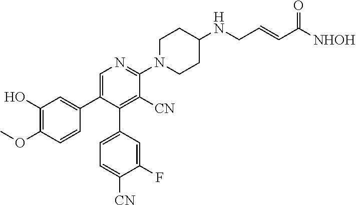 | ¹H NMR (400 MHz, Methanol- d₄) δ 8.45 (s, 1H), 7.76 (dd, J = 8.0, 6.6 Hz, 1H), 7.38 (dd, J = 9.8, 1.4 Hz, 1H), 7.25 (dd, J = 8.0, 1.4 Hz, 1H), 6.92-6.70 (m, 2H), 6.52 (dd, J = 5.8, 2.2 Hz, 2H), 6.27 (d, J = 15.4 Hz, 1H), 4.45 (d, J = 13.6 Hz, 2H), 3.95 (d, J = 7.0 Hz, 2H), 3.82 (s, 3H), 3.50 (s, 1H), 3.20 (t, J = 12.6 Hz, 2H), 2.29 (d, J = 11.0 Hz, 2H), 1.85 (tt, J = 12.4, 6.2 Hz, 2H). | 543.2 |
| 52 | (E)-3-(4-(((1-(3- Cyano-4-(4-cyano-3- fluorophenyl)-5-(4- methoxyphenyl) pyridin- 2-yl)piperidin-4- yl)amino)methyl) phenyl)-N- hydroxyacrylamide- hydrochloride | 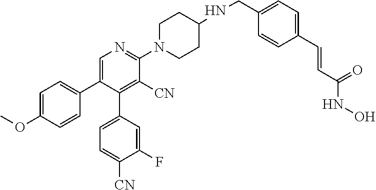 [SMILES: COc1ccc(-c2cnc(N3CCC(NCc4ccc(/C=C/C(=O)NO)cc4)CC3)c(C#N)c2-c2ccc(C#N)c(F)c2)cc1] | ¹H NMR (400 MHz, DMSO-d₆) δ 10.80 (s, 1H), 9.20 (s, 2H), 8.50 (s, 1H), 8.01-7.94 (m, 1H), 7.71- 7.58 (m, 5H), 7.48 (m, 1H), 7.30 (dd, J = 8.0, 1.4 Hz, 1H), 7.03-6.97 (m, 2H), 6.87-6.81 (m, 2H), 6.52 (d, J = 15.6 Hz, 1H), 4.40-4.18 (m, 5H), 3.71 (s, 3H), 3.40 (s, 2H), 3.13 (t, J = 12.6 Hz, 2H), 2.28 (d, J = 12.2 Hz, 2H), 1.79 (d, J = 11.8 Hz, 3H). | 603.3 |
| 53 | (E)-3-(4-(((1-(3- Cyano-4-(4-cyano-3- fluorophenyl)-5-(2- fluoro-3-hydroxy- phenyl)pyridin-2- yl)piperidin-4- yl)amino)methyl) phenyl)-N- hydroxyacrylamide- formate | 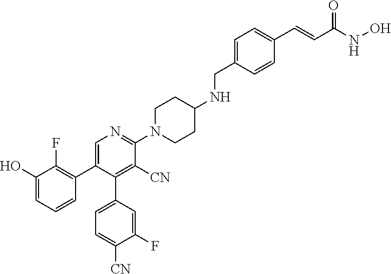 [SMILES: N#Cc1ccc(-c2c(-c3cccc(O)c3F)cnc(N3CCC(NCc4ccc(/C=C/C(=O)NO)cc4)CC3)c2C#N)cc1F] | ¹H NMR (400 MHz, DMSO-d₆) δ ppm: 8.41 (s, 1H), 8.38 (s, 2H), 7.93 (t, J = 7.4 Hz, 1H), 7.62 (dd, J = 10.2, 1.4 Hz, 1H), 7.50 (d, J = 7.8 Hz, 2H), 7.40 (d, J = 8.0 Hz, 3H), 7.27 (dd, J = 8.0, 1.4 Hz, 1H), 6.88 (dd, J = 6.2, 4.2 Hz, 2H), 6.58 (dq, J = 6.8, 3.4, 2.2 Hz, 1H), 6.46 (d, J = 15.8 Hz, 1H), 4.23 (d, J = 13.4 Hz, 2H), 3.79 (s, 2H), 3.22 (t, J = 12.0 Hz, 2H), 2.78-2.66 (m, 1H), 2.04- 1.93 (m, 2H), 1.44 (q, J = 11.0, 10.2 Hz, 2H). | 607.2 |

### Example 54

### Preparation of (E)-3-(4-(((1-(6-(4-cyano-3-fluorophenyl)-5-(3-hydroxy-4-methoxyphenyl)-4-methoxypyridin-2-yl)piperidin-4-yl)amino)methyl)phenyl)-N-hydroxyacrylamide formate

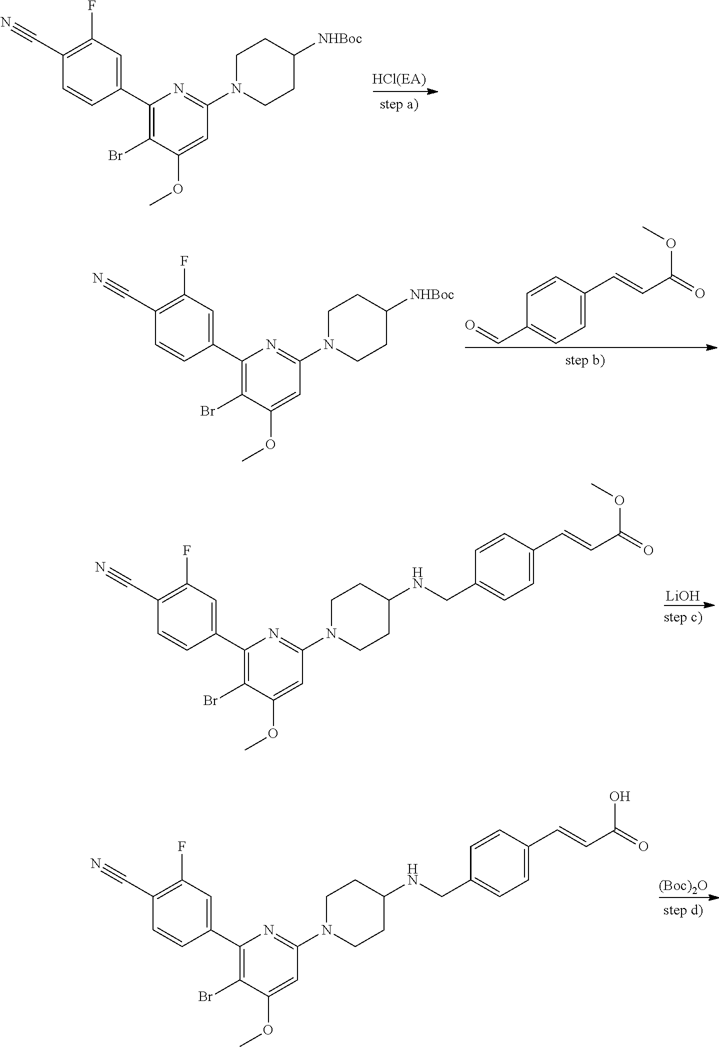

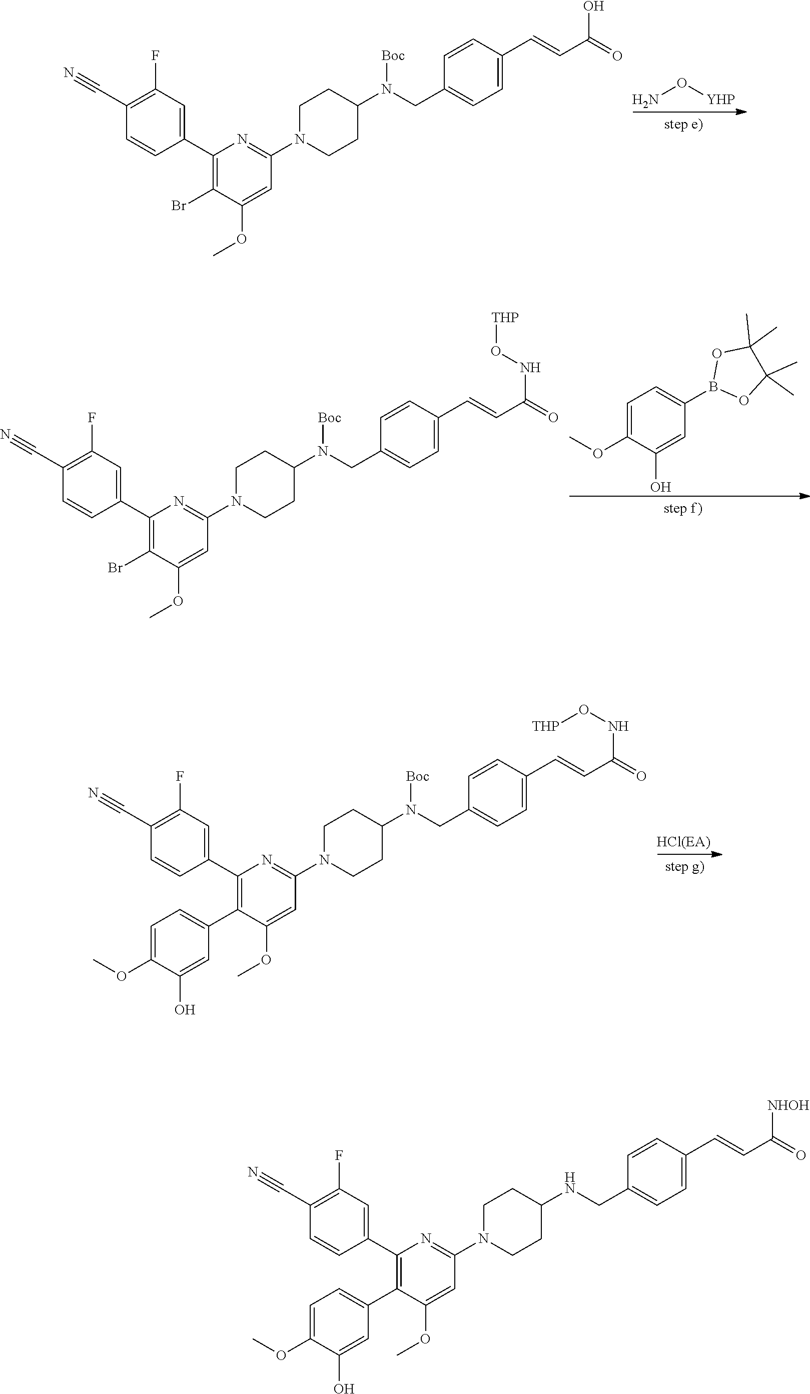

### Step a): preparation of 4-(6-(4-aminopiperidin-1-yl)-3-bromo-4-methoxypyridin-2-yl)-2-fluorobenzonitrile

The product of Step d) in Example 27: tert-butyl (1-(5-bromo-6-(4-cyano-3-fluorophenyl)-4-methoxypyridin-2-yl)piperidin-4-yl)carbamate (600 mg, 1.20 mmol) was added to HCl (EA) (4 mL) and stirred at room temperature for 1 hour. After removal of the solvent under reduced pressure, the crude product 4-(6-(4-aminopiperidin-1-yl)-3-bromo-4-methoxypyridin-2-yl)-2-fluorobenzonitrile was obtained with a yield of 41.81%.

ESI-MS m/z: 405.1 [M+H]⁺.

### Step b): preparation of methyl (E)-3-(4-(((1-(5-bromo-6-(4-cyano-3-fluorophenyl)-4-methoxypyridin-2-yl)piperidin-4-yl)amino)methyl)phenyl)acrylate

4-(6-(4-Aminopiperidin-1-yl)-3-bromo-4-methoxypyridin-2-yl)-2-fluorobenzonitrile (420 mg, 1.04 mmol) and methyl 4-formylcinnamate (356.05 mg, 1.87 mmol) were added to a mixture of DCE/MeOH/CH₃COOH=20:1:0.1 (8 mL), and the mixture was stirred at room temperature for 0.5 hours, then cooled in an ice bath, and sodium cyanoborohydride (326.77 mg, 5.2 mmol) was added. The reaction was slowly warmed to room temperature and reacted for 1 hour, and monitored by LCMS until the starting material was consumed by TLC (EA:PE=1:2). The reaction solution was poured into water and extracted with DCM. The organic phases were combined, and the solvent was removed. The residue was mixed with silica gel and purified by column chromatography on silica gel to afford methyl (E)-3-(4-(((1-(5-bromo-6-(4-cyano-3-fluorophenyl)-4-methoxypyridin-2-yl)piperidin-4-yl)amino)methyl)phenyl)acrylate with a yield of 79.65%.

ESI-MS m/z: 479.1 [M+H]⁺.

### Step c): preparation of (E)-3-(4-(((1-(5-bromo-6-(4-cyano-3-fluorophenyl)-4-methoxypyridin-2-yl)piperidin-4-yl)amino)methyl)phenyl)acrylic acid

methyl (E)-3-(4-(((1-(5-bromo-6-(4-cyano-3-fluorophenyl)-4-methoxypyridin-2-yl)piperidin-4-yl)amino)methyl)phenyl)acrylate (500 mg, 0.86 mmol) and lithium hydroxide (123.58 mg, 5.16 mmol) were added to a mixture of THF/H₂O=4:1 (10 mL), and the mixture was reacted at room temperature overnight, and monitored by LCMS until the starting materials were completely consumed. The reaction solution was acidified to pH 3 with dilute hydrochloric acid, and then extracted with EA. The organic phases were combined, and the solvent was removed under reduced pressure to afford the crude product (E)-3-(4-(((1-(5-bromo-6-(4-cyano-3-fluorophenyl)-4-methoxypyridin-2-yl)piperidin-4-yl)amino)methyl)phenyl)acrylic acid, which was directly used in the next step. ESI-MS m/z: 565.1 [M+H]⁺.

### Step d): preparation of (E)-3-(4-(((1-(5-bromo-6-(4-cyano-3-fluorophenyl)-4-methoxypyridin-2-yl)piperidin-4-yl)(tert-butoxycarbonyl)amino)methyl)phenyl)acrylic acid

(E)-3-(4-(((1-(5-bromo-6-(4-cyano-3-fluorophenyl)-4-methoxypyridin-2-yl)piperidin-4-yl)amino)methyl)phenyl)acrylic acid (300 mg, 0.53 mmol), di-tert-butyl dicarbonate (347.02 mg, 1.59 mmol), and sodium bicarbonate (222.63 mg, 2.65 mmol) were added to a mixture of THF/H₂O=4:1 (4 mL), and the solution was stirred at room temperature overnight and monitored by LCMS until the starting materials were completely consumed. Dilute hydrochloric acid was added to adjust the pH to 7, followed by extraction with EA, and the organic phases were combined, and the solvent was removed. The residue was mixed with silica gel and purified by column chromatography on silica gel to afford ((E)-3-(4-(((1-(5-bromo-6-(4-cyano-3-fluorophenyl)-4-methoxypyridin-2-yl)piperidin-4-yl)(tert-butoxycarbonyl)amino)methyl)phenyl)acrylic acid with a yield of 90.72%.

ESI-MS m/z: 664.2 [M+H]⁺.

### Step e): preparation of tert-butyl (E)-(1-(5-bromo-6-(4-cyano-3-fluorophenyl)-4-methoxypyridin-2-yl)piperidin-4-yl)(4-(3-oxo-3-((tetrahydro-2H-pyran-2-yl)oxy)amino)prop-1-en-1-yl)benzyl)carbamate

(E)-3-(4-(((1-(5-bromo-6-(4-cyano-3-fluorophenyl)-4-methoxypyridin-2-yl)piperidin-4-yl)(tert-butoxycarbonyl)amino)methyl)phenyl)acrylic acid (50 mg, 75 μmol), O-(tetrahydro-2H-pyran-2-yl)hydroxylamine (10.54 mg, 0.09 mmol), HATU (34.22 mg, 90 μmol), and diisopropylethylamine (29.08 mg, 0.22 mmol) were added to DMF (8 mL), and the reaction solution was stirred at room temperature for 0.5 h and monitored by LCMS until the starting materials were completely consumed. The reaction solution was poured into water, followed by extraction with EA. The organic phase was washed with saturated brine, and the solvent was removed under reduced pressure. The residue was mixed with silica gel and purified column chromatography on silica gel to afford tert-butyl (E)-(1-(5-bromo-6-(4-cyano-3-fluorophenyl)-4-methoxypyridin-2-yl)piperidin-4-yl)(4-(3-oxo-3-((tetrahydro-2H-pyran-2-yl)oxy)amino)prop-1-en-1-yl)benzyl)carbamate with a yield of 73.23%.

ESI-MS m/z: 764.2 [M+H]⁺.

### Step f): preparation of tert-butyl (E)-(1-(6-(4-cyano-3-fluorophenyl)-5-(3-hydroxy-4-methoxyphenyl)-4-methoxypyridin-2-yl)piperidin-4-yl)(4-(3-oxo-3-((tetrahydro-2H-pyran-2-yl)oxy)amino)prop-1-en-1-yl)benzyl)carbamate

Tert-butyl (E)-(1-(5-bromo-6-(4-cyano-3-fluorophenyl)-4-methoxypyridin-2-yl)piperidin-4-yl)(4-(3-oxo-3-((tetrahydro-2H-pyran-2-yl)oxy)amino)prop-1-en-1-yl)benzyl)carbamate (50 mg, 65 μmol), 2-methoxy-5-(tetramethyl-1,3,2-dioxaborolan-2-yl)phenol (19.51 mg, 78 μmol), [1,1′-bis(diphenylphosphino)ferrocene]dichloropalladium (II) (4.76 mg, 6.5 μmol), and cesium carbonate (38.12 mg, 0.12 mmol) were added to a mixture of 1,4-dioxane/H₂O=5:1 (4 mL). After purging with nitrogen, the reaction solution was heated to 100° C. and stirred for 1 hour, and LCMS indicated the disappearance of the starting material and. The solvent was removed, and the residue was mixed with silica gel and purified column chromatography on silica gel to afford tert-butyl (E)-(1-(6-(4-cyano-3-fluorophenyl)-5-(3-hydroxy-4-methoxyphenyl)-4-methoxypyridin-2-yl)piperidin-4-yl)(4-(3-oxo-3-((tetrahydro-2H-pyran-2-yl)oxy)amino)prop-1-en-1-yl)benzyl)carbamate with a yield of 66.65%.

ESI-MS m/z: 808.4 [M+H]⁺.

### Step g): preparation of (E)-3-(4-(((1-(6-(4-cyano-3-fluorophenyl)-5-(3-hydroxy-4-methoxyphenyl)-4-methoxypyridin-2-yl)piperidin-4-yl)amino)methyl)phenyl)-N-hydroxyacrylamide formate

Tert-butyl (E)-(1-(6-(4-cyano-3-fluorophenyl)-5-(3-hydroxy-4-methoxyphenyl)-4-methoxypyridin-2-yl)piperidin-4-yl)(4-(3-oxo-3-((tetrahydro-2H-pyran-2-yl)oxy)amino)prop-1-en-1-yl)benzyl)carbamate (150 mg, 0.19 mmol) was added to HCl (EA) (4M, 5 mL), and the mixture was stirred at room temperature for 0.5 hours, and monitored by LCMS until the starting materials were completely consumed. The solvent was removed, and the residue was purified by Prep-HPLC (separation method 3) to afford (E)-3-(4-(((1-(6-(4-cyano-3-fluorophenyl)-5-(3-hydroxy-4-methoxyphenyl)-4-methoxypyridin-2-yl)piperidin-4-yl)amino)methyl)phenyl)-N-hydroxyacrylamide formate with a yield of 90%.

¹H NMR (400 MHZ, DMSO-d₆) δ 10.74 (s, 1H), 8.28 (s, 1H), 7.73 (dd, J=8.0, 7.0 Hz, 1H), 7.51 (d, J=7.8 Hz, 2H), 7.48-7.36 (m, 3H), 7.27 (dd, J=11.0, 1.6 Hz, 1H), 7.19 (dd, J=8.0, 1.6 Hz, 1H), 6.78 (d, J=8.4 Hz, 1H), 6.54-6.44 (m, 2H), 6.42 (s, 1H), 6.37 (dd, J=8.2, 2.0 Hz, 1H), 4.29 (m, 2H), 3.81 (s, 2H), 3.77 (s, 3H), 3.73 (s, 3H), 3.04-2.83 (m, 2H), 2.77-2.63 (m, 1H), 2.02-1.86 (m, 2H), 1.33 (q, J=9.6 Hz, 2H).

ESI-MS m/z: 624.3 [M+H]⁺.

### Example 56

### Preparation of N¹-(1-(7-(4-cyano-3-fluorophenyl)-8-(3-hydroxy-4-methylphenyl)imidazo[1,2-c]pyrimidin-5-yl)piperidin-4-yl)-N⁸-hydroxyoctanediamide hydrochloride

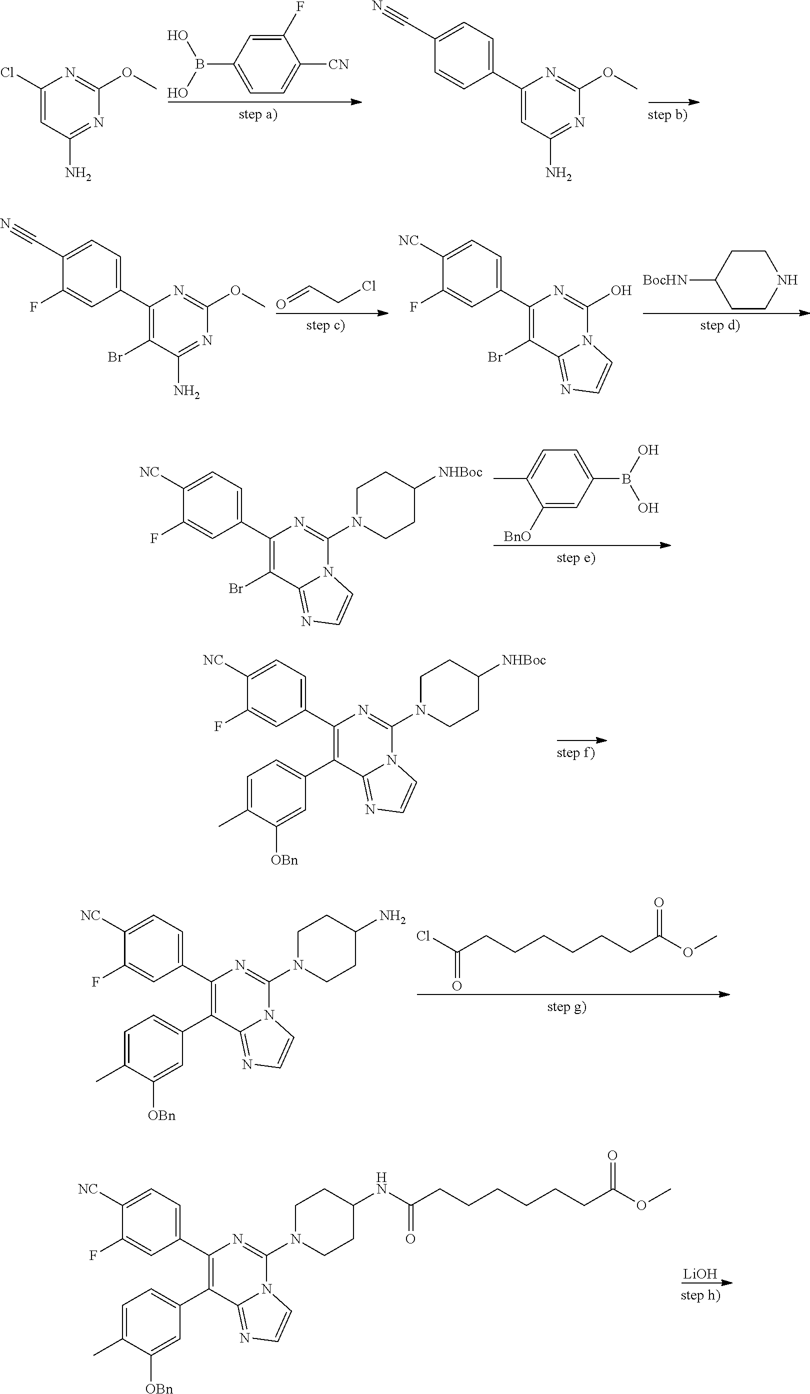

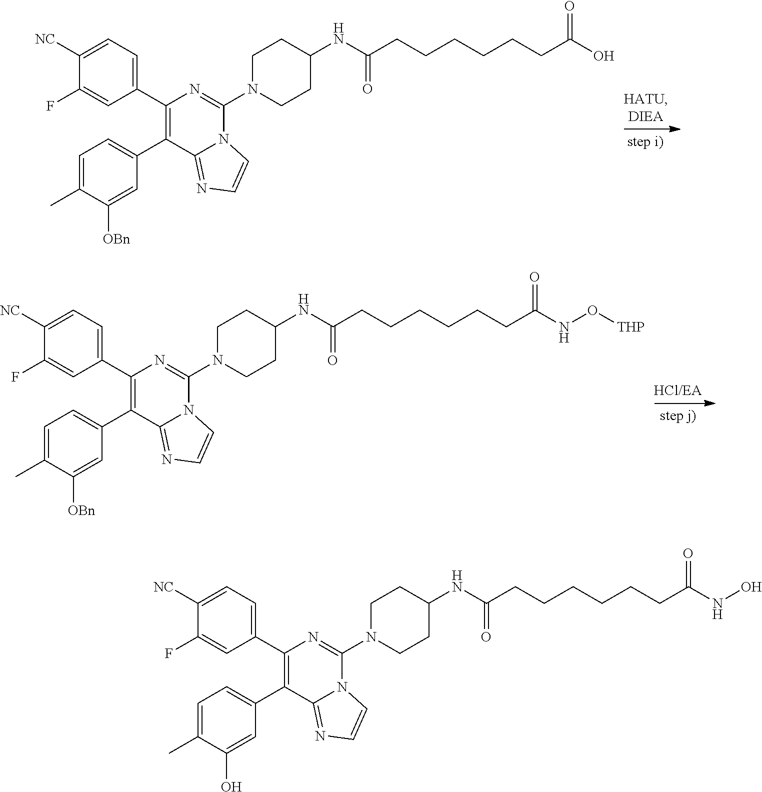

### Step a): preparation of 4-(6-amino-2-methoxypyrimidin-4-yl)-2-fluorobenzonitrile

6-Chloro-2-methoxypyrimidin-4-amine (24 g, 150.40 mmol), (4-cyano-3-fluorophenyl)boronic acid (29.77 g, 180.48 mmol), di-tert-butyl-(4-dimethylaminophenyl)phosphinopalladium (II) dichloride (5.32 g, 7.52 mmol), and sodium carbonate (47.82 g, 451.20 mmol) were added to a reaction solution containing 1,4-dioxane (350 mL) and water (70 mL), and the mixture was stirred at 95° C. for 2 hours. Afterward, water (150 mL) was added to the reaction solution, and the mixture was stirred at room temperature for 30 minutes, then filtered, and the filter cake was loaded into a 500 mL eggplant-shaped flask. Isopropanol (250 mL) was added, and the mixture was stirred at 50° C. for 30 minutes, then cooled to room temperature and stirred for an additional hour. After filtration, the filter cake was dried to afford 4-(6-amino-2-methoxypyrimidin-4-yl)-2-fluorobenzonitrile with a yield of 95%.

ESI-MS m/z=245.1 [M+H]⁺.

### Step b): preparation of 4-(6-amino-5-bromo-2-methoxypyrimidin-4-yl)-2-fluorobenzonitrile

4-(6-Amino-2-methoxypyrimidin-4-yl)-2-fluorobenzonitrile (24 g, 98.27 mmol) was added to a reaction solution of acetonitrile (240 mL) and DMSO (48 mL), and the mixture was cooled in an ice bath, and NBS (18.36 g, 103.18 mmol) was added, then the reaction solution was stirred at room temperature for 40 minutes. Then the reaction mixture was filtered, and the filtrate was concentrated, and EA (300 mL) was added and stirred thoroughly before filtering again. The filter cake was washed with MTBE, and dried to afford 4-(6-amino-5-bromo-2-methoxypyrimidin-4-yl)-2-fluorobenzonitrile with a yield of 92%.

ESI-MS m/z=322.0 [M+H]⁺.

### Step c): preparation of 4-(8-bromo-5-hydroxyimidazo[1,2-c]pyrimidin-7-yl)-2-fluorobenzonitrile

4-(6-Amino-5-bromo-2-methoxypyrimidin-4-yl)-2-fluorobenzonitrile (34 g, 105.22 mmol) and 2-chloroacetaldehyde (103.25 g, 526.1 mmol) were added to a flask containing IPA (300 mL), and the mixture was stirred at 100° C. for 24 hours. The reaction solution was cooled to room temperature and stirred for 2 hours, then filtered. The filter cake was washed with MTBE (50 mL), and the filtrate was concentrated, and then isopropanol (70 mL) was added to the residue and stirred at 50° C. for 30 minutes, then cooled to room temperature and stirred for 2 more hours before filtration. The filter cake was washed with MTBE (70 mL), and the combined cakes from both filtrations were dried to afford 4-(8-bromo-5-hydroxyimidazo[1,2-c]pyrimidin-7-yl)-2-fluorobenzonitrile with a yield of 75%.

ESI-MS m/z=333.0 [M+H]⁺.

### Step d): preparation of tert-butyl (1-(8-bromo-7-(4-cyano-3-fluorophenyl)imidazo[1,2-c]pyrimidin-5-yl)piperidin-4-yl)carbamate

4-(8-Bromo-5-hydroxyimidazo[1,2-c]pyrimidin-7-yl)-2-fluorobenzonitrile (8 g, 24.02 mmol), BOP (21.25 g, 48.04 mmol), DIEA (12.42 g, 96.08 mmol), and tert-butyl piperidin-4-yl-carbamate (12.03 g, 60.05 mmol) were added to a reaction flask containing acetonitrile (80 mL), and the mixture was stirred at 60° C. for 16 hours. Silica gel was added to the reaction liquid, the solvent was removed and the residue was purified by column chromatography on silica gel (eluent: DCM/MeOH=10/1) to afford a crude product, which was further purified with a C₁₈ column to afford tert-butyl (1-(8-bromo-7-(4-cyano-3-fluorophenyl)imidazo[1,2-c]pyrimidin-5-yl)piperidin-4-yl)carbamate with a yield of 31%.

ESI-MS m/z=515.1 [M+H]⁺.

### Step e): preparation of tert-butyl (1-(8-(3-(benzyloxy)-4-methylphenyl)-7-(4-cyano-3-fluorophenyl)imidazo[1,2-c]pyrimidin-5-yl)piperidin-4-yl)carbamate

Tert-butyl (1-(8-bromo-7-(4-cyano-3-fluorophenyl)imidazo[1,2-c]pyrimidin-5-yl)piperidin-4-yl)carbamate (400 mg, 0.78 mmol), Pd(dppf)Cl₂ (57 mg, 78 μmol), cesium carbonate (0.51 g, 1.56 mmol), and (3-(benzyloxy)-4-methylphenyl)boronic acid (0.23 g, 0.94 mmol) were added to a reaction solution containing 1,4-dioxane (2 mL) and water (0.5 mL), and the mixture was stirred under microwave heating at 120° C. for 1 hour. The reaction solution was concentrated after adding silica gel, and the residue was then purified by silica gel chromatography (eluent: petroleum ether/ethyl acetate=1/1) afford to tert-butyl (1-(8-(3-(benzyloxy)-4-methylphenyl)-7-(4-cyano-3-fluorophenyl)imidazo[1,2-c]pyrimidin-5-yl)piperidin-4-yl)carbamate with a yield of 84%.

ESI-MS m/z=633.3 [M+H]⁺.

### Step f): preparation of 4-(5-(4-aminopiperidin-1-yl)-8-(3-(benzyloxy)-4-methylphenyl)imidazo[1,2-c]pyrimidin-7-yl)-2-fluorobenzonitrile

4.0M Hydrochloric acid solution in EA (25 mL) was added to a reaction flask containing tert-butyl (1-(8-(3-(benzyloxy)-4-methylphenyl)-7-(4-cyano-3-fluorophenyl)imidazo[1,2-c]pyrimidin-5-yl)piperidin-4-yl)carbamate (400 mg, 0.63 mmol), and the mixture was stirred at room temperature for 1 hour. The reaction solution was concentrated under reduced pressure to afford 4-(5-(4-aminopiperidin-1-yl)-8-(3-(benzyloxy)-4-methylphenyl)imidazo[1,2-c]pyrimidin-7-yl)-2-fluorobenzonitrile with a yield of 93%.

ESI-MS m/z=533.2 [M+H]⁺.

### Step g): Preparation of methyl 8-((1-(8-(3-(benzyloxy)-4-methylphenyl)-7-(4-cyano-3-fluorophenyl)imidazo[1,2-c]pyrimidin-5-yl)piperidin-4-yl)amino)-8-oxooctanoate

4-(5-(4-Aminopiperidin-1-yl)-8-(3-(benzyloxy)-4-methylphenyl)imidazo[1,2-c]pyrimidin-7-yl)-2-fluorobenzonitrile (420 mg, 0.79 mmol) was added to a reaction flask containing DCM (20 mL), and the mixture was cooled to 0° C., and then TEA (0.32 g, 3.16 mmol) and methyl 8-chloro-8-oxooctanoate (0.24 g, 1.19 mmol) were added sequentially. After stirring for 10 minutes, the mixture was cooled to room temperature and stirred at room temperature for an hour. The reaction was quenched by adding water (20 mL), then extracted with ethyl acetate (20 mL×2), and the organic phases were combined, washed with saturated brine (15 mL×2), dried over anhydrous sodium sulfate, filtered, and concentrated to afford methyl 8-((1-(8-(3-(benzyloxy)-4-methylphenyl)-7-(4-cyano-3-fluorophenyl)imidazo[1,2-c]pyrimidin-5-yl)piperidin-4-yl)amino)-8-oxooctanoate with a yield of 60%.

ESI-MS m/z=703.3 [M+H]⁺.

### Step h): preparation of 8-((1-(8-(3-(benzyloxy)-4-methylphenyl)-7-(4-cyano-3-fluorophenyl)imidazo[1,2-c]pyrimidin-5-yl)piperidin-4-yl)amino)-8-oxooctanoic acid

Methyl 8-((1-(8-(3-(benzyloxy)-4-methylphenyl)-7-(4-cyano-3-fluorophenyl)imidazo[1,2-c]pyrimidin-5-yl)piperidin-4-yl)amino)-8-oxooctanoate (500 mg, 0.71 mmol) and lithium hydroxide (29 mg, 0.71 mmol) were added to a reaction solution containing THF (10 mL), MeOH (6 mL), and water (4 mL), and the mixture was stirred at room temperature for 1 hour. The pH of the solution was adjusted to 4 with 2M HCl, and then water (20 mL) was added. The mixture was extracted with a mixture of DCM/MeOH (5:1) (20 mL×2), and the organic phases were combined, washed with saturated brine (20 mL×2), dried over anhydrous sodium sulfate, filtered, and concentrated to afford 8-((1-(8-(3-(benzyloxy)-4-methylphenyl)-7-(4-cyano-3-fluorophenyl)imidazo[1,2-c]pyrimidin-5-yl)piperidin-4-yl)amino)-8-oxooctanoic acid with a yield of 84%.

ESI-MS m/z=689.3 [M+H]⁺.

Step i): preparation of N¹-(1-(8-(3-(benzyloxy)-4-methylphenyl)-7-(4-cyano-3-fluorophenyl)imidazo[1,2-c]pyrimidin-5-yl)piperidin-4-yl)-N⁸-((tetrahydro-2H-pyran-2-yl)oxy)octanediamide

8-((1-(8-(3-(Benzyloxy)-4-methylphenyl)-7-(4-cyano-3-fluorophenyl)imidazo[1,2-c]pyrimidin-5-yl)piperidin-4-yl)amino)-8-oxooctanoic acid (520 mg, 0.75 mmol), O-(tetrahydro-2H-pyran-2-yl)hydroxylamine (130 mg, 1.13 mmol), HATU (430 mg, 1.13 mmol), and DIEA (480 mg, 3.75 mmol) were added to a reaction solution containing DMF (15 mL), and the mixture was stirred at room temperature for 1 hour. The reaction solution was quenched by adding water (20 mL), then extracted with ethyl acetate (20 mL×2), and the organic phases were combined, washed with saturated brine (15 mL×2), dried over anhydrous sodium sulfate, filtered, and the residue was purified by column chromatography on silica gel (eluent: DCM/MeOH=8/1) to afford N¹-(1-(8-(3-(benzyloxy)-4-methylphenyl)-7-(4-cyano-3-fluorophenyl)imidazo[1,2-c]pyrimidin-5-yl)piperidin-4-yl)-N⁸-((tetrahydro-2H-pyran-2-yl)oxy)octanediamide with a yield of 48%.

ESI-MS m/z=788.4 [M+H]⁺.

### Step j): Preparation of N¹-(1-(7-(4-cyano-3-fluorophenyl)-8-(3-hydroxy-4-methylphenyl)imidazo[1,2-c]pyrimidin-5-yl)piperidin-4-yl)-N⁸-hydroxyoctanediamide hydrochloride

4.0M Hydrochloric acid solution in EA (6 mL) was added to a reaction flask containing N¹-(1-(8-(3-(benzyloxy)-4-methylphenyl)-7-(4-cyano-3-fluorophenyl)imidazo[1,2-c]pyrimidin-5-yl)piperidin-4-yl)-N⁸-((tetrahydro-2H-pyran-2-yl)oxy)octanediamide (120 mg, 0.15 mmol), and the mixture was stirred at room temperature for 1 hour. After concentrated, it was purified by Prep-HPLC (separation method 1) to afford N¹-(1-(7-(4-cyano-3-fluorophenyl)-8-(3-hydroxy-4-methylphenyl)imidazo[1,2-c]pyrimidin-5-yl)piperidin-4-yl)-N⁸-hydroxyoctanediamide hydrochloride with a yield of 22%.

¹H NMR (400 MHZ, DMSO-d₆) δ ppm 10.36 (s, 1H), 9.72 (s, 1H), 8.24 (d, J=2.2 Hz, 1H), 8.14 (s, 1H), 7.98-7.85 (m, 2H), 7.53 (m, 1H), 7.42-7.31 (m, 1H), 7.20 (d, J=7.8 Hz, 1H), 6.81 (s, 1H), 6.70-6.59 (m, 1H), 4.02 (d, J=13.2 Hz, 2H), 3.93 (d, J=8.6 Hz, 1H), 3.32 (t, J=12.2 Hz, 2H), 2.19 (s, 3H), 2.08 (t, J=7.4 Hz, 2H), 1.99-1.87 (m, 4H), 1.69 (q, J=11.2 Hz, 2H), 1.47 (p, J=7.6 Hz, 4H), 1.32-1.16 (m, 4H).

ESI-MS m/z=614.3 [M+H]⁺.

### Example 57

The preparation of N¹-(4-(5-(4-aminopiperidin-1-yl)-7-(4-cyano-3-fluorophenyl)imidazo[1,2-c]pyrimidin-8-yl)-2-hydroxyphenyl)-N⁸-hydroxyoctanediamine diformate was prepared according to the synthetic method of Example 56 (separation method 1), and the structure and characterization data are as follows:

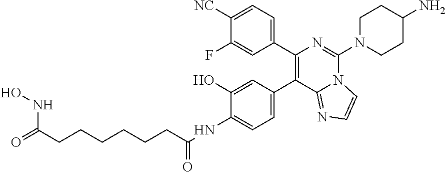 [SMILES: N#Cc1ccc(-c2nc(N3CCC(N)CC3)n3ccnc3c2-c2ccc(NC(=O)CCCCCCC(=O)NO)c(O)c2)cc1F]

¹H NMR (400 MHz, Methanol-d₄) δ ppm 8.45 (s, 2H), 7.73 (d, J=1.6 Hz, 1H), 7.67 (d, J=8.2 Hz, 1H), 7.56-7.43 (m, 2H), 7.40 (dd, J=10.8, 1.6 Hz, 1H), 7.24 (dd, J=8.2, 1.6 Hz, 1H), 6.78 (d, J=1.2 Hz, 1H), 6.66 (dd, J=8.2, 1.8 Hz, 1H), 4.46 (d, J=21.8 Hz, 1H), 4.02 (d, J=13.2 Hz, 2H), 3.19-3.02 (m, 2H), 2.37 (t, J=7.4 Hz, 2H), 2.15-1.93 (m, 4H), 1.91-1.75 (m, 2H), 1.59 (m, 4H), 1.32 (dq, J=8.8, 4.8 Hz, 4H).

ESI-MS m/z=615.3 [M+H]⁺

### Example 58

### Preparation of N¹-(4-(6-(4-aminopiperidin-1-yl)-5-cyano-4-(4-cyano-3-fluorophenyl)pyridin-3-yl)-2-hydroxyphenyl)-N⁸-hydroxyoctanediamide hydrochloride

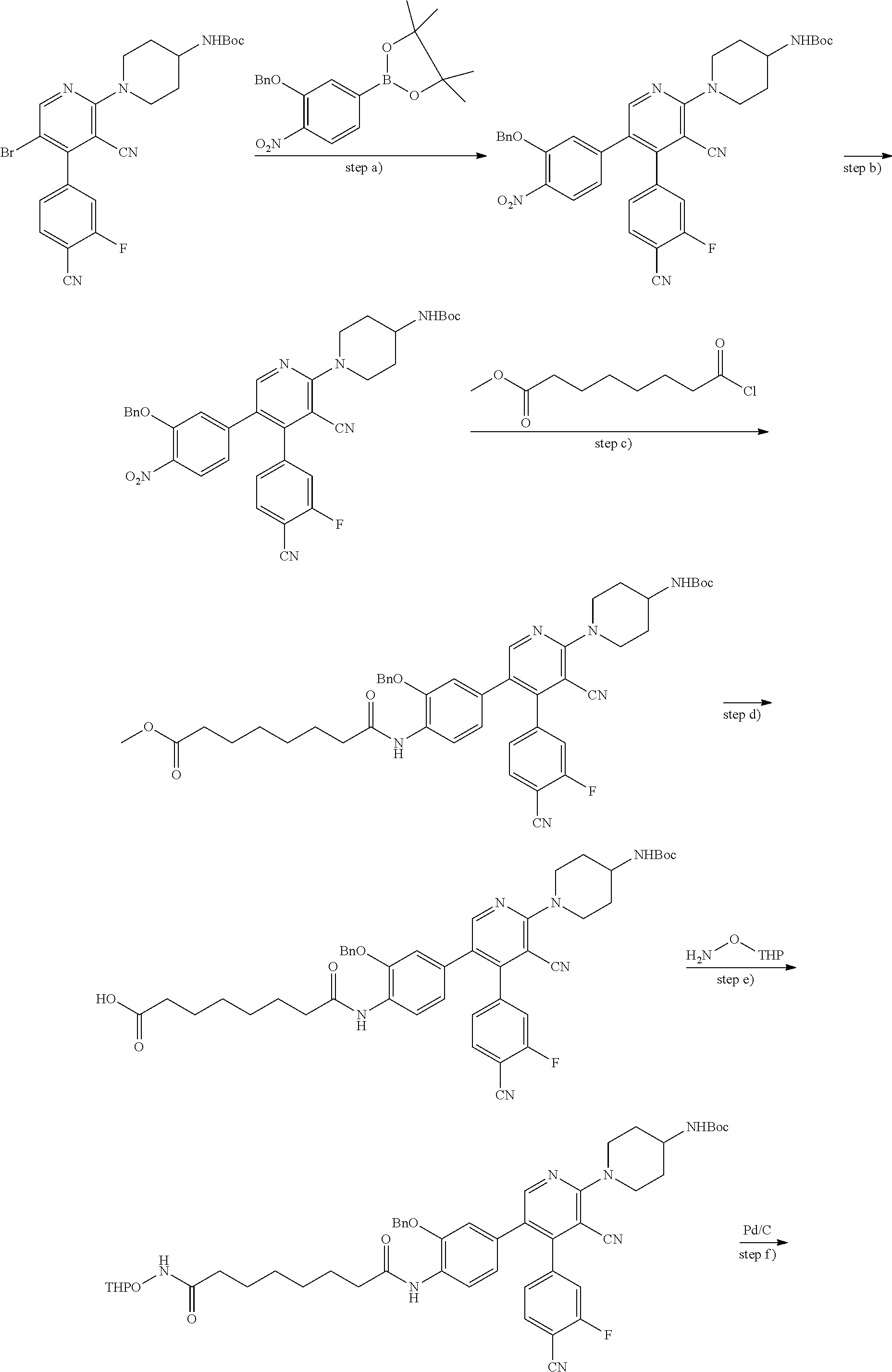

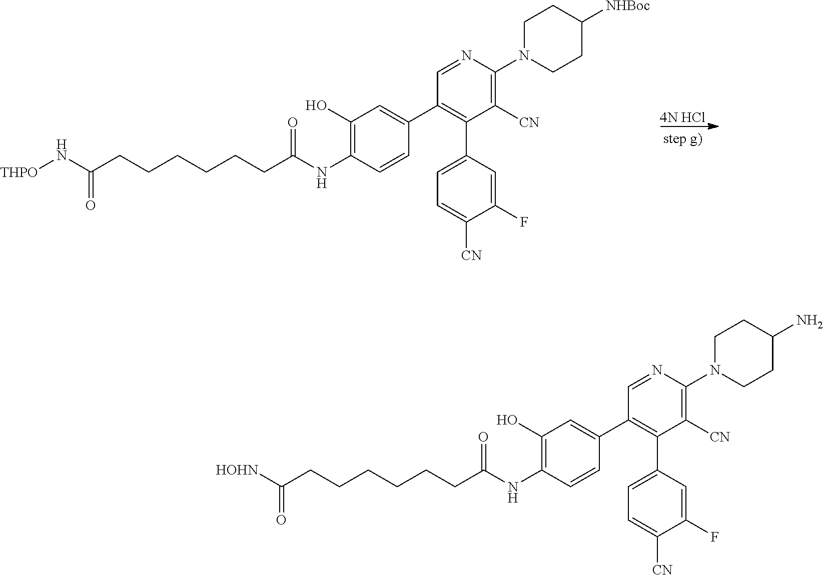

### Step a): preparation of tert-butyl (1-(5-(3-(benzyloxy)-4-nitrophenyl)-3-cyano-4-(4-cyano-3-fluorophenyl)pyridin-2-yl)piperidin-4-yl)carbamate

The product of Step c) in Example 37: tert-butyl (1-(5-bromo-3-cyano-4-(4-cyano-3-fluorophenyl)pyridin-2-yl)piperidin-4-yl)carbamate (200 mg, 401 μmol), 2-(3-(Benzyloxy)-4-nitrophenyl)-4,4,5,5-tetramethyl-1,3,2-dioxaborolane (213 mg, 601 μmol), Cs₂CO₃ (261 mg, 802 μmol), Pd(dppf)Cl₂ (29 mg, 40 μmol), 1,4-dioxane (4 mL), and H₂O (1 mL) were added to a reaction flask and stirred at 120° C. for 1 hour. The reaction solution was concentrated under reduced pressure, and the residue was purified by silica gel chromatography (eluent: petroleum ether/ethyl acetate=1/2) to afford tert-butyl (1-(5-(3-(benzyloxy)-4-nitrophenyl)-3-cyano-4-(4-cyano-3-fluorophenyl)pyridin-2-yl)piperidin-4-yl)carbamate with a yield of 78.5%.

ESI-MS (m/z)=529.3 [M+H]⁺.

### Step b): preparation of tert-butyl (1-(5-(4-amino-3-(benzyloxy)phenyl)-3-cyano-4-(4-cyano-3-fluorophenyl)pyridin-2-yl)piperidin-4-yl)carbamate

Tert-butyl (1-(5-(3-(benzyloxy)-4-nitrophenyl)-3-cyano-4-(4-cyano-3-fluorophenyl)pyridin-2-yl)piperidin-4-yl)carbamate (167 mg, 315 μmol), zinc powder (202 mg, 3.1 mmol), ammonium chloride (164 mg, 3.1 mmol), and THF (5 mL) were added to a reaction flask, and the mixture was heated to 65° C. and reacted for 12 hours. After the reaction was completed, the reaction solution was cooled to room temperature, then filtered, and the filtrate was concentrated under reduced pressure. The residue was purified by silica gel chromatography (eluent: petroleum ether/ethyl acetate=1/2) to afford tert-butyl (1-(5-(4-amino-3-(benzyloxy)phenyl)-3-cyano-4-(4-cyano-3-fluorophenyl)pyridin-2-yl)piperidin-4-yl)carbamate with a yield of 72.3%.

ESI-MS (m/z)=619.3 [M+H]⁺.

### Step c): preparation of methyl 8-((2-(benzyloxy)-4-(6-(4-(tert-butoxycarbonyl)amino)piperidin-1-yl)-5-cyano-4-(4-cyano-3-fluorophenyl)pyridin-3-yl)phenyl)amino)-8-oxooctanoate

Tert-butyl (1-(5-(4-amino-3-(benzyloxy)phenyl)-3-cyano-4-(4-cyano-3-fluorophenyl)pyridin-2-yl)piperidin-4-yl)carbamate (140 mg, 227 μmol), methyl 8-chloro-8-oxooctanoate (93 mg, 454 μmol), triethylamine (46 mg, 454 μmol), and DCM (5 mL) were added to a reaction flask and stirred at room temperature for 2 hours. The mixture was concentrated under reduced pressure, and the residue was purified by silica gel chromatography (eluent: petroleum ether/ethyl acetate=1/1) to afford methyl 8-((2-(benzyloxy)-4-(6-(4-(tert-butoxycarbonyl)amino)piperidin-1-yl)-5-cyano-4-(4-cyano-3-fluorophenyl)pyridin-3-yl)phenyl)amino)-8-oxooctanoate with a yield of 58.0%.

ESI-MS (m/z)=789.4 [M+H]⁺.

### Step d): preparation of 8-((2-(benzyloxy)-4-(6-(4-(tert-butoxycarbonyl)amino)piperidin-1-yl)-5-cyano-4-(4-cyano-3-fluorophenyl)pyridin-3-yl)phenyl)amino)-8-oxooctanoic acid

Methyl 8-((2-(benzyloxy)-4-(6-(4-(tert-butoxycarbonyl)amino)piperidin-1-yl)-5-cyano-4-(4-cyano-3-fluorophenyl)pyridin-3-yl)phenyl)amino)-8-oxooctanoate (104 mg, 132 μmol), lithium hydroxide monohydrate (28 mg, 660 μmol), tetrahydrofuran (2 mL), isopropanol (2 mL), and water (1 mL) were added to a reaction flask and stirred at room temperature for 12 hours. Under ice bath cooling, 1N concentrated hydrochloric acid was added dropwise to adjust the pH to 3˜4, and then water (10 mL) was added, and the mixture was extracted with ethyl acetate (10 mL×3). The organic phases were combined, washed with saturated brine (10 mL×2), and concentrated to dryness under reduced pressure to afford 8-((2-(benzyloxy)-4-(6-(4-(tert-butoxycarbonyl)amino)piperidin-1-yl)-5-cyano-4-(4-cyano-3-fluorophenyl)pyridin-3-yl)phenyl)amino)-8-oxooctanoic acid, which was used directly for the next reaction step.

ESI-MS (m/z)=775.4 [M+H]⁺.

Step e): preparation of tert-butyl (1-(5-(3-(benzyloxy)-4-(8-oxo-8-((tetrahydro-2H-pyran-2-yl)oxy)amino)octanamino)phenyl)-3-cyano-4-(4-cyano-3-fluorophenyl)pyridin-2-yl)piperidin-4-yl)carbamate 8-((2-(Benzyloxy)-4-(6-(4-(tert-butoxycarbonyl)amino)piperidin-1-yl)-5-cyano-4-(4-cyano-3-fluorophenyl)pyridin-3-yl)phenyl)amino)-8-oxooctanoic acid (102 mg, 132 μmol), O-(tetrahydro-2H-pyran-2-yl)hydroxylamine (31 mg, 264 μmol), DIEA (34 mg, 264 μmol), and DMF (2 mL) were added to a reaction flask. HATU (62 mg, 158 μmol) was added under stirring at room temperature, and the reaction was maintained for 1 hour at room temperature. After the reaction was completed, water (10 mL) was added to quench the reaction, followed by extraction with ethyl acetate (20 mL×2), and the organic phases were combined, washed sequentially with saturated sodium bicarbonate aqueous solution (20 mL×1) and saturated brine (10 mL×2), dried over anhydrous sodium sulfate, filtered, and the filtrate was concentrated under reduced pressure. The residue was purified by silica gel chromatography (eluent: petroleum ether/ethyl acetate=1/1) to afford tert-butyl (1-(5-(3-(benzyloxy)-4-(8-oxo-8-((tetrahydro-2H-pyran-2-yl)oxy)amino)octanamino)phenyl)-3-cyano-4-(4-cyano-3-fluorophenyl)pyridin-2-yl)piperidin-4-yl)carbamate with a yield of 60.5%.

ESI-MS (m/z)=874.4 [M+H]⁺.

### Step f): preparation of tert-butyl (1-(3-cyano-4-(4-cyano-3-fluorophenyl)-5-(3-hydroxy-4-(8-oxo-8-((tetrahydro-2H-pyran-2-yl)oxy)amino)octanamino)phenyl)pyridin-2-yl)piperidin-4-yl)carbamate

Tert-butyl (1-(5-(3-(benzyloxy)-4-(8-oxo-8-((tetrahydro-2H-pyran-2-yl)oxy)amino)octanamino)phenyl)-3-cyano-4-(4-cyano-3-fluorophenyl)pyridin-2-yl)piperidin-4-yl)carbamate (70 mg, 80 μmol), palladium on carbon (15 mg, 5%), and ethanol (5 mL) were sequentially added to a reaction flask, and the mixture was stirred and purged with hydrogen three times, then stirred at room temperature for 2 hours under hydrogen atmosphere. After the reaction was completed, the solution was filtered, and the filtrate was concentrated under reduced pressure to afford tert-butyl (1-(3-cyano-4-(4-cyano-3-fluorophenyl)-5-(3-hydroxy-4-(8-oxo-8-((tetrahydro-2H-pyran-2-yl)oxy)amino)octanamino)phenyl)pyridin-2-yl)piperidin-4-yl)carbamate with a yield of 85.0%. ESI-MS (m/z)=784.4 [M+H]⁺.

### Step g): preparation of N¹-(4-(6-(4-aminopiperidin-1-yl)-5-cyano-4-(4-cyano-3-fluorophenyl)pyridin-3-yl)-2-hydroxyphenyl)-N⁸-hydroxyoctanediamide hydrochloride

Tert-butyl (1-(3-cyano-4-(4-cyano-3-fluorophenyl)-5-(3-hydroxy-4-(8-oxo-8-((tetrahydro-2H-pyran-2-yl)oxy)amino)octanamino)phenyl)pyridin-2-yl)piperidin-4-yl)carbamate (53 mg, 68 μmol) was added to a reaction flask, followed by adding hydrogen chloride solution in ethyl acetate (4M, 2.5 mL), and the mixture was stirred at room temperature for 1 hour, resulting in the precipitation of a solid. After concentrated under reduced pressure, the crude product was purified by Prep-HPLC (separation method 1) to afford N¹-(4-(6-(4-aminopiperidin-1-yl)-5-cyano-4-(4-cyano-3-fluorophenyl)pyridin-3-yl)-2-hydroxyphenyl)-N⁸-hydroxyoctanediamide hydrochloride with a yield of 21.5%.

¹H NMR (400 MHZ, Methanol-d₄) δ 8.47 (s, 1H), 7.77 (dd, J=7.8, 6.6 Hz, 1H), 7.60 (d, J=8.2 Hz, 1H), 7.41 (dd, J=9.6, 1.4 Hz, 1H), 7.25 (dd, J=8.0, 1.4 Hz, 1H), 6.65-6.43 (m, 2H), 4.44 (d, J=13.6 Hz, 2H), 3.45 (ddt, J=11.4, 8.6, 4.4 Hz, 1H), 3.23 (s, 2H), 2.44 (t, J=7.4 Hz, 2H), 2.26-2.05 (m, 4H), 1.94-1.55 (m, 6H), 1.41 (dd, J=7.6, 4.0 Hz, 4H).

ESI-MS (m/z)=500.3 [M+H]⁺.

Examples 59-60 were prepared similarly according to the synthetic method of Example 58 (the separation method for the compounds: hydrochloride and formate were prepared according to separation method 1 and 3, respectively), and the structures and characterization data are as follows:

| Ex- | | | | MS |
|---|---|---|---|---|
| am- | | | | (M + |
| ple | Chemical name | Structure | ¹H NMR | H)⁺ |
| 59 | (E)-3-(3-(N-(1-(3-Cyano- 4-(4-cyano-3- fluorophenyl)-5-(3- hydroxy-4- methoxyphenyl)pyridin- 2-yl)piperidin-4- yl)sulfamoyl)phenyl)-N- hydroxyacrylamide hydrochloride | 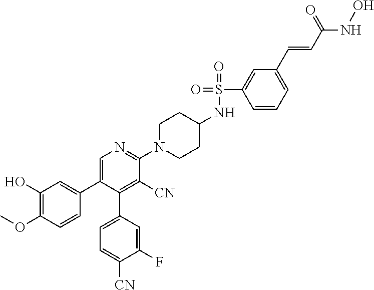 [SMILES: COc1ccc(-c2cnc(N3CCC(NS(=O)(=O)c4cccc(/C=C/C(=O)NO)c4)CC3)c(C#N)c2-c2ccc(C#N)c(F)c2)cc1O] | ¹H NMR (400 MHz, Methanol-d₄) δ 8.36 (s, 1H), 8.07 (s, 1H), 7.91 (dd, J = 7.8, 1.6 Hz, 1H), 7.88-7.77 (m, 1H), 7.78-7.57 (m, 3H), 7.34 (dd, J = 9.8, 1.6 Hz, 1H), 7.20 (dd, J = 8.0, 1.6 Hz, 1H), 6.88-6.75 (m, 1H), 6.59 (d, J = 15.8 Hz, 1H), 6.54-6.40 (m, 2H), 4.14 (d, J = 13.4 Hz, 2H), 3.80 (s, 3H), 3.40 (td, J = 10.2, 5.2 Hz, 1H), 3.25- 3.07 (m, 2H), 1.91-1.80 (m, 2H), 1.66 (d, J = 7.0 Hz, 2H). | 669.2 |
| 60 | (E)-N-(1-(3-Cyano-4-(4- cyano-3-fluorophenyl)-5- (3-hydroxy-4- methoxyphenyl)pyridin- 2-yl)piperidin-4-yl)-4-(3- (hydroxyamino)-3- oxoprop-1-en-1- yl)benzamide formate | 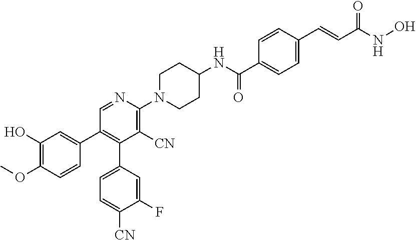 [SMILES: COc1ccc(-c2cnc(N3CCC(NC(=O)c4ccc(/C=C/C(=O)NO)cc4)CC3)c(C#N)c2-c2ccc(C#N)c(F)c2)cc1O] | ¹H NMR (400 MHz, DMSO- d₆) δ 10.75 (s, 1H), 9.03 (s, 1H), 8.45 (s, 1H), 8.43 (d, J = 8.0 Hz, 1H), 7.97 (dd, J = 8.0, 6.9 Hz, 1H), 7.90 (d, J = 8.2 Hz, 2H), 7.74-7.60 (m, 3H), 7.48 (d, J = 15.8 Hz, 1H), 7.29 (dd, J = 8.0, 1.6 Hz, 1H), 6.82 (d, J = 8.0 Hz, 1H), 6.55 (d, J = 15.8 Hz, 1H), 6.47 (s, 2H), 4.27 (d, J = 12.8 Hz, 2H), 4.21-4.05 (m, 1H), 3.72 (s, 3H), 3.22 (t, J = 12.0 Hz, 2H), 2.05-1.91 (m, 2H), 1.85-1.67 (m, 2H). | 633.2 |

### Example 61

### Preparation of 4-(6-(4-aminopiperidin-1-yl)-5-cyano-4-(4-cyano-3-fluorophenyl)pyridin-3-yl)-N-hydroxybenzamide hydrochloride

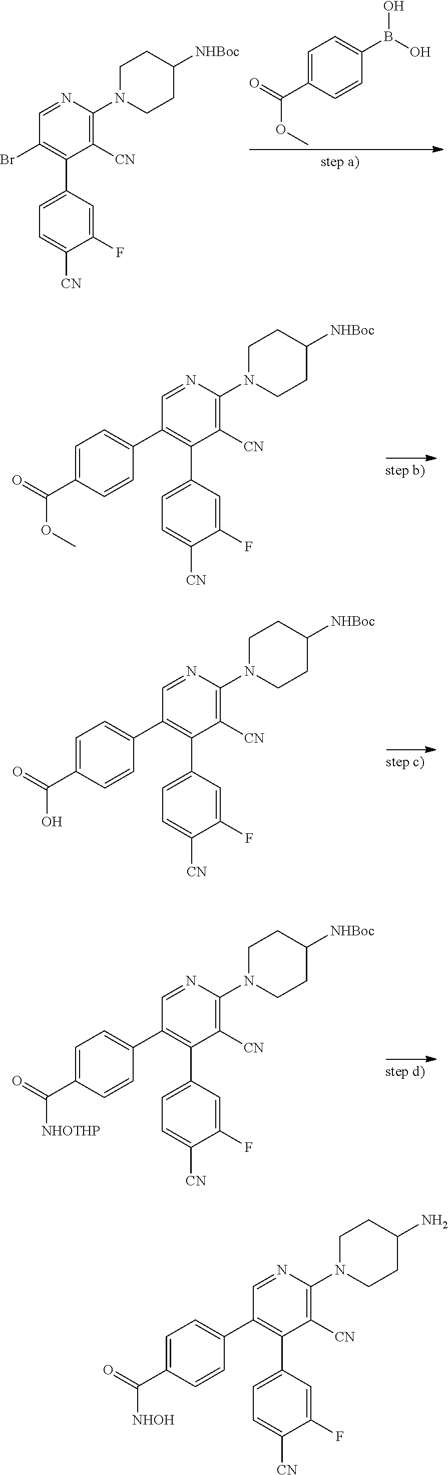

### Step a): preparation of methyl 4-(6-(4-{(tert-butoxy)carbonyl]amino}piperidin-1-yl)-5-cyano-4-(4-cyano-3-fluorophenyl)pyridin-3-yl)benzoate

The product of Step c) in Example 37: tert-butyl (1-(5-bromo-3-cyano-4-(4-cyano-3-fluorophenyl)pyridin-2-yl)piperidin-4-yl)carbamate (100 mg, 0.20 mmol), (4-(methoxycarbonyl)phenyl)boronic acid (53.99 mg, 0.30 mmol), Cs₂CO₃ (130.33 mg, 0.40 mmol), and Pd(dppf)Cl₂ (14.63 mg, 0.02 mmol) were dissolved in a mixture of water (1 mL) and 1,4-dioxane (4 mL), the reaction solution was stirred under microwave heating at 120° C. for 1 hour under nitrogen. The mixture was concentrated under reduced pressure to obtain a crude product, and the residue was then purified by silica gel chromatography (eluent: petroleum ether/ethyl acetate=10/7) to afford methyl 4-(6-(4 {(tert-butoxy)carbonyl]amino}piperidin-1-yl)-5-cyano-4-(4-cyano-3-fluorophenyl)pyridin-3-yl)benzoate with a yield of 94%.

ESI-MS m/z=556.2 [M+H]⁺.

### Step b): preparation of 4-(6-(4-{(tert-butoxy)carbonyl]amino}piperidin-1-yl)-5-cyano-4-(4-cyano-3-fluorophenyl)pyridin-3-yl)benzoic acid

methyl 4-(6-(4-{(tert-butoxy)carbonyl]amino}piperidin-1-yl)-5-cyano-4-(4-cyano-3-fluorophenyl)pyridin-3-yl)benzoate (105 mg, 0.19 mmol) and lithium hydroxide monohydrate (80 mg, 1.9 mmol) were dissolved in a solution of tetrahydrofuran (2.5 mL) and water (1 mL) and reacted for 16 hours. Water (20 mL) was added, and the pH was adjusted to 3-4 with 1N HCl aqueous solution. The mixture was then extracted with ethyl acetate (20 mL×2), and the organic phases were combined, washed with saturated brine (15 mL×2), dried over anhydrous sodium sulfate, filtered, and concentrated to obtain a crude product, which was directly used in the next step.

ESI-MS m/z=542.2 [M+H]⁺

### Step c): preparation of tert-butyl (1-(3-cyano-4-(4-cyano-3-fluorophenyl)-5-(4-(((tetrahydro-2H-pyran-2-yl)oxy)carbamoyl)phenyl)pyridin-2-yl)piperidin-4-yl)carbamate

4-(6-(4 {(Tert-butoxy)carbonyl]amino)piperidin-1-yl)-5-cyano-4-(4-cyano-3-fluorophenyl)pyridin-3-yl)benzoic acid (100 mg, 0.18 mmol) and O-(tetrahydro-2H-pyran-2-yl)hydroxylamine (64 mg, 0.54 mmol) were dissolved in N,N-dimethylformamide (5 mL). To the solution were added DIPEA (120 mg, 0.93 mmol) and HATU (210 mg, 0.55 mmol), and the solution was stirred for 30 minutes. Upon completion of the reaction, water (20 mL) was added to quench the reaction, followed by extraction with ethyl acetate (20 mL×2), and the organic phases were combined, washed with saturated brine (15 mL×2), dried over anhydrous sodium sulfate, filtered, and concentrated to obtain a crude product, which was directly used in the next step.

ESI-MS m/z=641.3 [M+H]⁺.

### Step d): preparation of 4-(6-(4-aminopiperidin-1-yl)-5-cyano-4-(4-cyano-3-fluorophenyl)pyridin-3-yl)-N-hydroxybenzamide

tert-butyl (1-(3-cyano-4-(4-cyano-3-fluorophenyl)-5-(4-(((tetrahydro-2H-pyran-2-yl)oxy)carbamoyl)phenyl)pyridin-2-yl)piperidin-4-yl)carbamate (110 mg, 0.17 mmol) was dissolved in 4N HCl (6 mL) and reacted for 30 minutes. After the completion of the reaction, the mixture was concentrated to obtain a crude product, which was prepared by method 1 to afford 4-(6-(4-aminopiperidin-1-yl)-5-cyano-4-(4-cyano-3-fluorophenyl)pyridin-3-yl)-N-hydroxybenzamide hydrochloride with a yield of 62%. ¹H NMR (400 MHZ, Methanol-d₄) δ ppm 8.49 (s, 1H), 7.74 (dd, J=7.8, 6.6 Hz, 1H), 7.66 (d, J=7.8 Hz, 2H), 7.47-7.34 (m, 1H), 7.24 (m, 3H), 4.49 (d, J=13.2 Hz, 2H), 3.48 (tt, J=11.0, 4.0 Hz, 1H), 3.25 (d, J=11.2 Hz, 1H), 3.00 (s, 1H), 2.31-2.11 (m, 2H), 1.83 (qd, J=12.2, 3.8 Hz, 2H). ESI-MS m/z=457.2 [M+H]⁺

Examples 62-64 were prepared similarly according to the synthetic method of Example 61 (the compound separation method for the compound: hydrochloride and formate were prepared by separation methods 1 and 3, respectively), and the structure and characterization data are as follows:

| Exam- | | | | MS |
|---|---|---|---|---|
| ple | Chemical name | Structure | ¹H NMR | (M + H)⁺ |
| 62 | (E)-3-(4-(6-(4- Aminopiperidin-1- yl)-5-cyano-4-(4- cyano-3- fluorophenyl) pyridin-3-yl)-2- hydroxyphenyl)- N- hydroxyacrylamide hydrochloride | 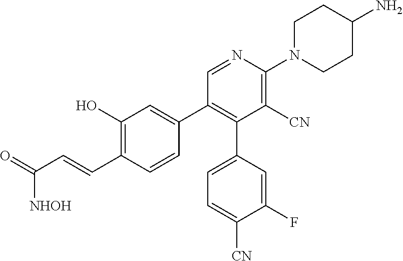 | ¹H NMR (400 MHz, Methanol-d₄) δ 8.47 (s, 1H), 7.86-7.69 (m, 2H), 7.44 (dd, J = 9.8, 1.6 Hz, 1H), 7.36 (d, J = 8.4 Hz, 1H), 7.24 (dd, J = 8.0, 1.6 Hz, 1H), 6.68 (m, 1H), 6.62-6.54 (m, 2H), 4.46 (dp, J = 14.0, 2.2 Hz, 2H), 3.45 (ddt, J = 11.4, 8.6, 4.2 Hz, 1H), 3.24 (m, 2H), 2.26-2.04 (m, 2H), 1.82 (qd, J = 12.4, 4.2 Hz, 2H). | 499.2 |
| 63 | (E)-3-(4-(6-(4- Aminopiperidin-1- yl)-5-cyano-4-(4- cyano-3- fluorophenyl) pyridin-3- yl)phenyl)-N- hydroxyacrylamide diformate | 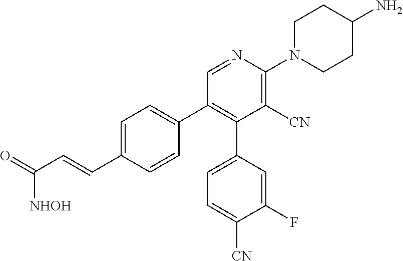 | ¹H NMR (400 MHz, Methanol-d₄) δ 8.56 (s, 2H), 8.47 (s, 1H), 7.70 (t, J = 8.0 Hz, 1H), 7.49-7.34 (m, 4H), 7.27 (d, J = 8, 1H) 7.19 (d, J = 8.0 Hz, 2H),, 6.48 (d, J = 15.8 Hz, 1H), 4.41 (d, J = 13.6 Hz, 2H), 3.30 (s, 1H), 3.22-3.05 (m, 2H), 2.09 (d, J = 12.3 Hz, 2H), 1.71 (qd, J = 12.3, 4.1 Hz, 2H). | 483.2 |
| 64 | (E)-3-(4-(6-(4- Amino-4- methylpiperidin-1- yl)-5-cyano-4-(4- cyano-3- fluorophenyl) pyridin-3- yl)-2- hydroxyphenyl)- N- hydroxyacrylamide formate | 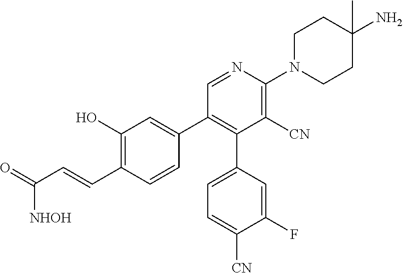 | ¹H NMR (400 MHz, DMSO-d₆) δ ppm: 10.68 (s, 1H), 10.16 (s, 1H), 8.94 (s, 1H), 8.50 (s, 1H), 7.97 (dd, J = 8.0, 6.8 Hz, 1H), 7.75 (dd, J = 10.0, 1.6 Hz, 1H), 7.54 (d, J = 15.8 Hz, 1H), 7.31 (d, J = 8.2 Hz, 1H), 7.27 (dd, J = 8.0, 1.6 Hz, 1H), 6.62-6.54 (m, 2H), 6.50 (d, J = 15.8 Hz, 1H), 4.03 (d, J = 14.0 Hz, 2H), 3.54 (q, J = 10.4, 10.0 Hz, 2H), 1.87 (d, J = 9.2 Hz, 2H), 1.80 (d, J = 13.8 Hz, 2H), 1.39 (s, 3H). | 513.2 |

### Example 66

### Preparation of (E)-3-(4-(((1-(4-(4-cyano-3-fluorophenyl)-5-(3-fluoro-4-methoxyphenyl)-1-methyl-6-oxo-1,6-dihydropyrimidin-2-yl)piperidin-4-yl)amino)methyl)phenyl)-N-hydroxyacrylamide formate

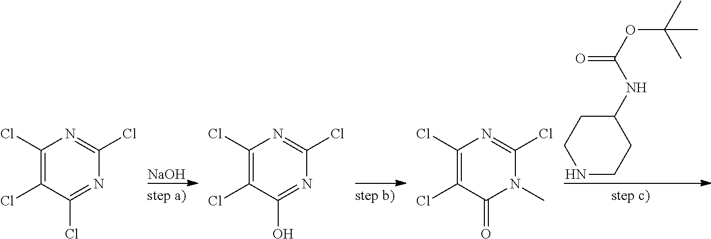

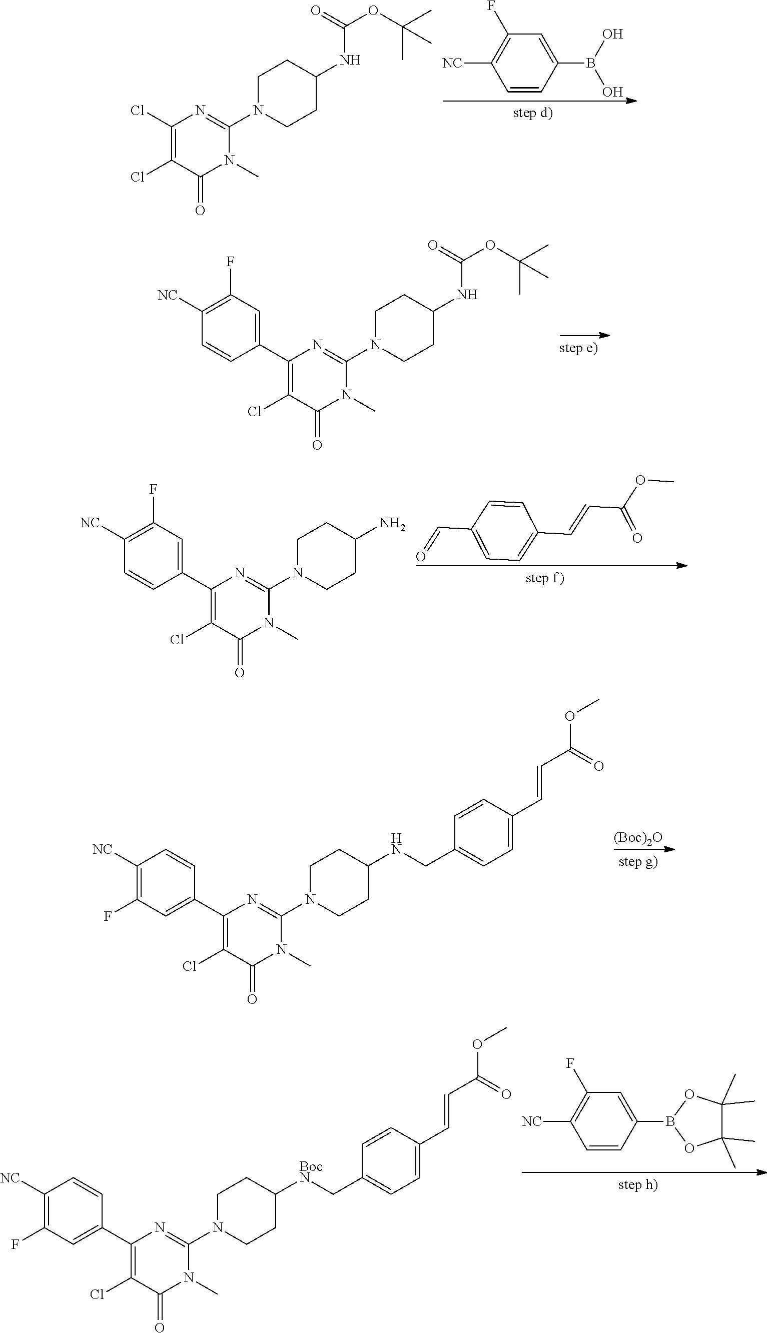

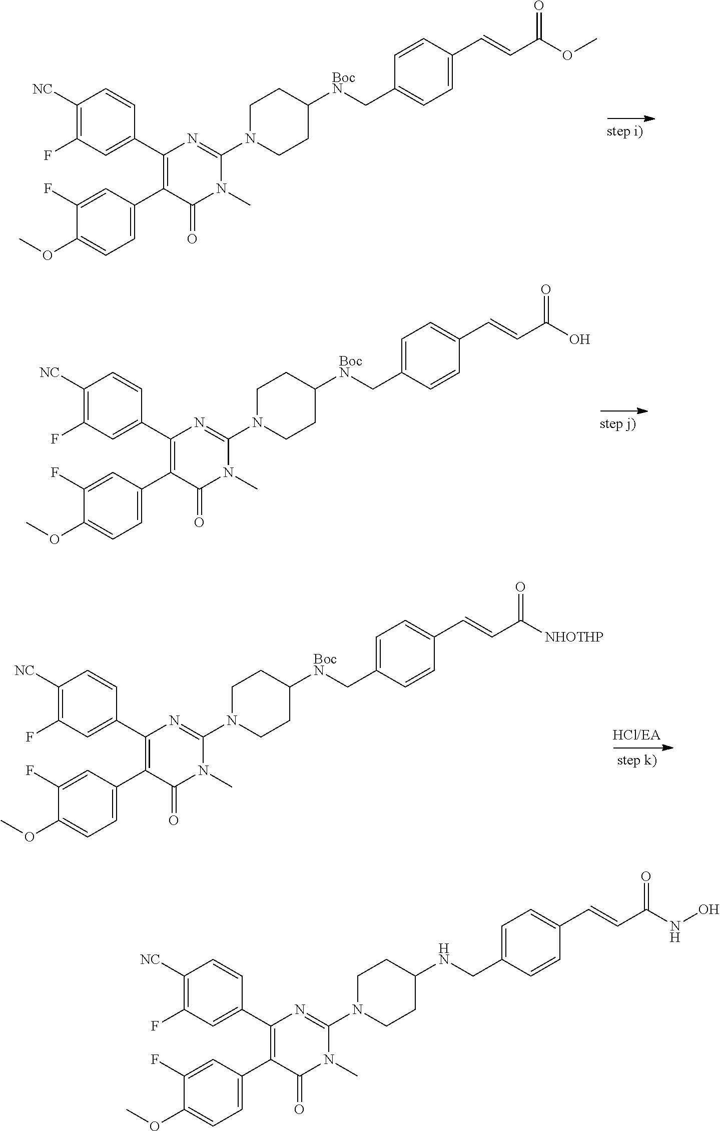

### Step a): Synthesis of 2,5,6-trichloropyrimidin-4-ol

Sodium hydroxide (1.19 g, 29.84 mmol) was dissolved in water (15 mL), and then added dropwise to a solution of 2,4,5,6-tetrachloropyrimidine (5 g, 22.95 mmol) in tetrahydrofuran (40 mL), and the mixture was stirred for 16 hours under nitrogen. After the reaction was completed as indicated by LCMS, dilute hydrochloric acid was added to acidify the solution, followed by extraction with ethyl acetate (20 mL×3 times), and the organic phases were combined, washed with saturated brine (15 mL), dried with anhydrous sodium sulfate, filtered, and then concentrated to dryness under reduced pressure. The residue was purified by trituration with diethyl ether, affording 2,5,6-trichloro-4-pyrimidinol with a yield of 74.1%.

ESI-MS m/z: 199.0 [M+H]⁺;

### Step b): synthesis of 2,5,6-trichloro-3-methylpyrimidin-4(3H)-one

To the solution containing 2,5,6-Trichloropyrimidin-4-ol (3.3 g, 16.6 mmol) and potassium carbonate (3.43 g, 79.6 mmol) in DMF (30 mL), was added iodomethane (4.7 g, 33.1 mmol) slowly. After the addition, the reaction was stirred at room temperature for 6 hours. Water (200 mL) was then added, followed by extraction with ethyl acetate (50 mL×3). The organic layer was washed with saturated brine (100 mL), dried with anhydrous sodium sulfate, filtered, and then concentrated under vacuum. The residue was purified by silica gel chromatography to afford 2,5,6-trichloro-3-methylpyrimidin-4(3H)-one with a yield of 82.5%.

ESI-MS m/z=213.0 [M+H]⁺;

### Step c): synthesis of tert-butyl (1-(4,5-dichloro-1-methyl-6-oxo-1,6-dihydropyrimidin-2-yl)piperidin-4-yl)carbamate

2,5,6-Trichloro-3-methylpyrimidin-4(3H)-one (2.5 g, 11.71 mmol), tert-butyl piperidin-4-yl-carbamate (2.35 g, 11.71 mmol), and DIPEA (3.03 g, 23.42 mmol) were dissolved in NMP (20 mL), and the solution was heated to 130° C. for 2 hours. After the reaction was completed as monitored by LC-MS, the reaction solution was cooled to room temperature, and then water (50 mL) was added. The mixture was extracted with ethyl acetate (30 mL×3 times), and the organic layers were combined, washed with saturated brine (20 mL), dried with anhydrous sodium sulfate, filtered, and concentrated under vacuum. The residue was purified by silica gel chromatography to afford tert-butyl (1-(4,5-dichloro-1-methyl-6-oxo-1,6-dihydropyrimidin-2-yl)piperidin-4-yl)carbamate with a yield of 67.4%.

ESI-MS m/z=213.0 [M+H]⁺;

### Step d): synthesis of tert-butyl (1-(5-chloro-4-(4-cyano-3-fluorophenyl)-1-methyl-6-oxo-1,6-dihydropyrimidin-2-yl)piperidin-4-yl)carbamate

Tert-butyl (1-(4,5-dichloro-1-methyl-6-oxo-1,6-dihydropyrimidin-2-yl)piperidin-4-yl)carbamate (1 g, 2.65 mmol), (4-cyano-3-fluorophenyl)boronic acid (570 mg, 3.44 mmol), cesium carbonate (1.73 g, 5.3 mmol), and dppf palladium dichloride (190 mg, 0.27 mmol) were dissolved in 1,4-dioxane (15 mL), then water (2 mL) was added, and the mixture was subjected to microwave heating at 120° C. for 60 minutes under nitrogen. After the reaction was completed as indicated by LCMS, water (10 mL) was added to dilute the solution, followed by extraction with ethyl acetate (20 mL×3). The organic phases were combined, washed with saturated brine (15 mL×2), dried with anhydrous sodium sulfate, filtered, and concentrated to dryness under reduced pressure. The residue was purified by silica gel column chromatography to afford tert-butyl (1-(5-chloro-4-(4-cyano-3-fluorophenyl)-1-methyl-6-oxo-1,6-dihydropyrimidin-2-yl)piperidin-4-yl)carbamate with a yield of 70.7%.

ESI-MS m/z: 462.2 [M+H]⁺;

### Step e): synthesis of 4-(2-(4-aminopiperidin-1-yl)-5-chloro-1-methyl-6-oxo-1,6-dihydropyrimidin-4-yl)-2-fluorobenzonitrile

Tert-butyl (1-(5-chloro-4-(4-cyano-3-fluorophenyl)-1-methyl-6-oxo-1,6-dihydropyrimidin-2-yl)piperidin-4-yl)carbamate (920 mg, 1.99 mmol) was added to 4N HCl (EA) (10 mL) solution and was stirred at room temperature for 0.5 hours and monitored by LCMS until the starting materials were completely consumed. The reaction solution was then poured into a saturated aqueous solution of sodium bicarbonate, and EA was added for extraction. The organic phase was washed with saturated brine, and the solvent was removed under reduced pressure to afford the crude product 4-(2-(4-aminopiperidin-1-yl)-5-chloro-1-methyl-6-oxo-1,6-dihydropyrimidin-4-yl)-2-fluorobenzonitrile with a yield of 91.2%.

ESI-MS m/z: 362.1 [M+H]⁺;

### Step f): synthesis of methyl (E)-3-(4-((1-(5-chloro-4-(4-cyano-3-fluorophenyl)-1-methyl-6-oxo-1,6-dihydropyrimidin-2-yl)piperidin-4-yl)amino)methyl)phenyl)acrylate

4-(2-(4-Aminopiperidin-1-yl)-5-chloro-1-methyl-6-oxo-1,6-dihydropyrimidin-4-yl)-2-fluorobenzonitrile (679 mg, 1.88 mmol) and methyl (E)-3-(4-formylphenyl)acrylate (536.36 mg, 2.82 mmol) were added to a mixture of DCE:MeOH:CH₃COOH=20:1:0.1 (10 mL), and the mixture was stirred at room temperature for 0.5 hours, then cooled in an ice bath, and sodium cyanoborohydride (590.7 mg, 9.40 mmol) was added. The reaction solution was slowly warmed back to room temperature and stirred for 1 hour, and the reaction was monitored by LCMS until the starting materials were completely consumed. The reaction solution was poured into water and extracted with DCM, and the organic phases were combined. The solvent was removed, and the residue was mixed with silica gel and purified by column chromatography on silica gel to afford methyl (E)-3-(4-((1-(5-chloro-4-(4-cyano-3-fluorophenyl)-1-methyl-6-oxo-1,6-dihydropyrimidin-2-yl)piperidin-4-yl)amino)methyl)phenyl)acrylate with a yield of 76.5%.

ESI-MS m/z: 539.2 [M+H]⁺.

### Step g): synthesis of methyl (E)-3-(4-((tert-butoxycarbonyl)(1-(5-chloro-4-(4-cyano-3-fluorophenyl)-1-methyl-6-oxo-1,6-dihydropyrimidin-2-yl)piperidin-4-yl)amino)methyl)phenyl)acrylate

Methyl (E)-3-(4-((1-(5-chloro-4-(4-cyano-3-fluorophenyl)-1-methyl-6-oxo-1,6-dihydropyrimidin-2-yl)piperidin-4-yl)amino)methyl)phenyl)acrylate (1.2 g, 2.24 mmol) was dissolved in a mixture solvent of THF: H₂O=3:1 (12 mL). After thorough stirring, (Boc)₂O (1.47 g, 6.72 mmol) and sodium bicarbonate (0.94 g, 11.20 mmol) were added, and the mixture was stirred at room temperature for one hour. After the reaction was completed, 0.5N hydrochloric acid aqueous solution (10 mL) was added, followed by extraction with ethyl acetate (10 mL×3), and the organic phases were combined, washed with saturated brine (10 mL×2), and the organic phase was concentrated to dryness under reduced pressure. The residue was then purified by silica gel chromatography to afford methyl (E)-3-(4-((tert-butoxycarbonyl)(1-(5-chloro-4-(4-cyano-3-fluorophenyl)-1-methyl-6-oxo-1,6-dihydropyrimidin-2-yl)piperidin-4-yl)amino)methyl)phenyl)acrylate with a yield of 89.0%.

ESI-MS (m/z)=636.2 [M+H]⁺;

### Step h): synthesis of methyl (E)-3-(4-((tert-butoxycarbonyl)(1-(4-(4-cyano-3-fluorophenyl)-5-(3-fluoro-4-methoxyphenyl)-1-methyl-6-oxo-1,6-dihydropyrimidin-2-yl)piperidin-4-yl)amino)methyl)phenyl)acrylate

Methyl (E)-3-(4-((tert-butoxycarbonyl)(1-(5-chloro-4-(4-cyano-3-fluorophenyl)-1-methyl-6-oxo-1,6-dihydropyrimidin-2-yl)piperidin-4-yl)amino)methyl)phenyl)acrylate (200 mg, 0.31 mmol), cesium carbonate (303.0 mg, 0.93 mmol), 2-fluoro-4-(4,4,5,5-tetramethyl-1,3,2-dioxaborolan-2-yl)benzonitrile (66.5 mg, 0.4 mmol), and [1,1′-bis(diphenylphosphino)ferrocene]palladium dichloride (21.8 mg, 0.031 mmol) were dissolved in a mixture of 1,4-dioxane (4 mL) and water (0.5 mL). The reaction solution was heated by microwave to 110° C. for 35 minutes under nitrogen, then water was added to dilute the mixture, which was then extracted with ethyl acetate (15 mL×3). The organic phases were combined, washed with saturated brine (10 mL×2), dried with anhydrous sodium sulfate, filtered, and concentrated under reduced pressure. The residue was purified by silica gel chromatography to afford methyl (E)-3-(4-((tert-butoxycarbonyl)(1-(4-(4-cyano-3-fluorophenyl)-5-(3-fluoro-4-methoxyphenyl)-1-methyl-6-oxo-1,6-dihydropyrimidin-2-yl)piperidin-4-yl)amino)methyl)phenyl)acrylate with a yield of 80.9%.

ESI-MS m/z: 726.3 [M+H]⁺;

### Step i): synthesis of (E)-3-(4-(tert-butoxycarbonyl)(1-(4-(4-cyano-3-fluorophenyl)-5-(3-fluoro-4-methoxyphenyl)-1-methyl-6-oxo-1,6-dihydropyrimidin-2-yl)piperidin-4-yl)amino)methyl)phenyl)acrylic acid

Methyl (E)-3-(4-((tert-butoxycarbonyl)(1-(4-(4-cyano-3-fluorophenyl)-5-(3-fluoro-4-methoxyphenyl)-1-methyl-6-oxo-1,6-dihydropyrimidin-2-yl)piperidin-4-yl)amino)methyl)phenyl)acrylate (225.1 mg, 0.31 mmol) was dissolved in a mixture of tetrahydrofuran:water=3:1 (4 mL), and lithium hydroxide (74.3 mg, 3.1 mmol) was added. And the mixture was stirred for 30 minutes and extracted with ethyl acetate (10 mL×3), and the organic phases were combined, washed with saturated brine (10 mL×2), dried with anhydrous sodium sulfate, filtered, and concentrated under reduced pressure to afford (E)-3-(4-(tert-butoxycarbonyl)(1-(4-(4-cyano-3-fluorophenyl)-5-(3-fluoro-4-methoxyphenyl)-1-methyl-6-oxo-1,6-dihydropyrimidin-2-yl)piperidin-4-yl)amino)methyl)phenyl)acrylic acid with a yield of 93.1%.

ESI-MS m/z: 712.3 [M+H]⁺;

### Step j): synthesis of tert-butyl (E)-(1-(4-(4-cyano-3-fluorophenyl)-5-(3-fluoro-4-methoxyphenyl)-1-methyl-6-oxo-1,6-dihydropyrimidin-2-yl)piperidin-4-yl)(4-(3-oxo-3-((tetrahydro-2H-pyran-2-yloxy)amino)prop-1-en-1-yl)benzyl)carbamate

(E)-3-(4-(Tert-butoxycarbonyl)(1-(4-(4-cyano-3-fluorophenyl)-5-(3-fluoro-4-methoxyphenyl)-1-methyl-6-oxo-1,6-dihydropyrimidin-2-yl)piperidin-4-yl)amino)methyl)phenyl)acrylic acid (200 mg, 0.28 mmol), O-(tetrahydro-2H-pyran-2-yl)hydroxylamine (39.4 mg, 0.34 mmol), HATU (127.8 mg, 0.34 mmol), and DIEA (108.6 mg, 0.84 mmol) were added to a reaction flask containing DMF (5 mL), and the mixture was stirred at room temperature for 1 hour. The reaction was quenched by adding water (10 mL), followed by extraction with ethyl acetate (10 mL×2). The organic phases were combined, washed with saturated brine (15 mL×2), dried with anhydrous sodium sulfate, filtered, and the residue was purified by silica gel chromatography to afford tert-butyl (E)-(1-(4-(4-cyano-3-fluorophenyl)-5-(3-fluoro-4-methoxyphenyl)-1-methyl-6-oxo-1,6-dihydropyrimidin-2-yl)piperidin-4-yl)(4-(3-oxo-3-((tetrahydro-2H-pyran-2-yloxy)amino)prop-1-en-1-yl)benzyl)carbamate with a yield of 75.2%.

ESI-MS m/z=810.3 [M+H]⁺;

### Step k): synthesis of (E)-3-(4-((1-(4-(4-cyano-3-fluorophenyl)-5-(3-fluoro-4-methoxyphenyl)-1-methyl-6-oxo-1,6-dihydropyrimidin-2-yl)piperidin-4-yl)amino)methyl)phenyl)-N-hydroxyacrylamide formate

4.0M Hydrochloric acid solution in EA (6 mL) was added to a reaction flask containing tert-butyl (E)-(1-(4-(4-cyano-3-fluorophenyl)-5-(3-fluoro-4-methoxyphenyl)-1-methyl-6-oxo-1,6-dihydropyrimidin-2-yl)piperidin-4-yl)(4-(3-oxo-3-((tetrahydro-2H-pyran-2-yloxy)amino)prop-1-en-1-yl)benzyl)carbamate (180 mg, 0.29 mmol), and the solution was stirred at room temperature for 1 hour. The reaction solution was concentrated and then purified by Prep-HPLC (separation method 3) to afford (E)-3-(4-((1-(4-(4-cyano-3-fluorophenyl)-5-(3-fluoro-4-methoxyphenyl)-1-methyl-6-oxo-1,6)-dihydropyrimidin-2-yl)piperidin-4-yl)amino)methyl)phenyl)-N-hydroxyacrylamide formate with a yield of 25%.

ESI-MS m/z=627.3 [M+H]⁺.

¹H NMR (400 MHZ, DMSO-d₆) δ 10.71 (s, 1H), 9.01 (s, 1H), 8.21-8.16 (m, 1H), 7.80 (dd, J=8.1, 6.9 Hz, 1H), 7.51 (d, J=7.9 Hz, 2H), 7.42-7.38 (m, 3H), 7.20 (dd, J=8.1, 1.4 Hz, 1H), 7.08-6.96 (m, 2H), 6.78 (dt, J=8.4, 1.4 Hz, 1H), 6.43 (d, J=15.8 Hz, 1H), 3.80 (m, 5H), 3.62 (d, J=13.0 Hz, 2H), 3.43 (m, 4H), 2.93 (t, J=11.9 Hz, 2H), 1.95 (d, J=12.6 Hz, 2H), 1.48 (q, J=11.0 Hz, 2H).

Example 67 is prepared following the synthesis method of Example 66 (compound separation method 3), and the structure and characterization data are as follows:

| Exam- | Chemical | | | MS |
|---|---|---|---|---|
| ple | name | Structure | ¹H NMR | (M + H)⁺ |
| 67 | (E)-3-(4- (((1-(4-(4- Cyano-3- fluorophenyl)- 5-(3- hydroxy-4- methoxy- phenyl)-1- methyl-6- oxo-1,6- dihydro- pyrimidin-2- yl)piperidin-4- yl)amino )methyl) phenyl)-N- hydroxy- acrylamide formate | 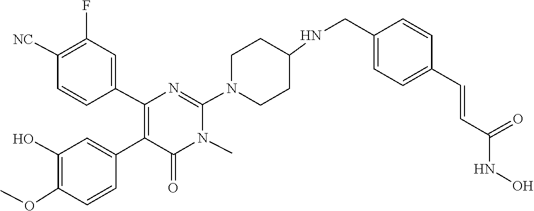 [SMILES: COc1ccc(-c2c(-c3ccc(C#N)c(F)c3)nc(N3CCC(NCc4ccc(/C=C/C(=O)NO)cc4)CC3)n(C)c2=O)cc1O] | ¹H NMR (400 MHz, DMSO- d₆) δ 10.71 (s, 1H), 8.89 (s, 1H), 8.19 (s, 1H), 7.78 (t, J = 8.0, 1H), 7.51 (d, J = 7.9 Hz, 2H), 7.23 (dd, J = 8.1, 1.4 Hz, 2H), 6.78 (d, J = 8.4 Hz, 2H), 6.61 (d, J = 2.1 Hz, 1H), 6.45 (s, 1H), 6.43 (s, 1H), 6.43 (s, 1H), 6.41 (d, J = 2.3 Hz, 1H), 3.79 (s, 2H), 3.73 (s, 3H), 3.60 (m, 3H), 2.91 (t, J = 11.8 Hz, 3H), 2.74-2.61 (m, 2H), 1.95 (d, J = 10.2 Hz, 2H), 1.48 (d, J = 11.2 Hz, 2H). | 625.3 |

### Example 69

(E)-3-(4-(((2-(4-Aminopiperidin-1-yl)-6-(4-cyano-3-fluorophenyl)pyridin-4-yl)oxy)methyl)phenyl)-N-hydroxyacrylamide formate was prepared similarly according to the synthetic method of Example 31, and the structure and characterization data are as follows:

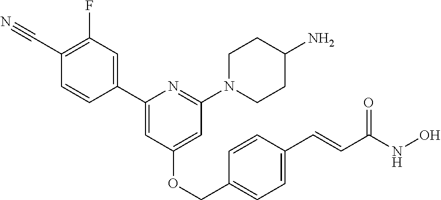 [SMILES: N#Cc1ccc(-c2cc(OCc3ccc(/C=C/C(=O)NO)cc3)cc(N3CCC(N)CC3)n2)cc1F]

¹H NMR (400 MHZ, DMSO+D2O-d6)₈ ppm: 8.41 (s, 1H), 8.20-8.06 (m, 2H), 7.99 (t, J=7.6 Hz, 1H), 7.60 (d, J=8.0 Hz, 2H), 7.55-7.40 (m, 3H), 7.14 (s, 1H), 6.58-6.44 (m, 2H), 5.28 (s, 2H), 4.38 (d, J=13.2 Hz, 2H), 3.08 (m, 1H), 3.02-2.89 (m, 2H), 1.89 (d, J=11.6 Hz, 2H), 1.38 (q, J=11.6 Hz, 2H).

ESI-MS m/z=488.2 [M+H]⁺.

### Example 70

(E)-3-(4-(((1-(7-(4-Cyano-3-fluorophenyl)-8-(3-hydroxy-4-methoxyphenyl)imidazo[1,2-c]pyrimidin-5-yl)piperidin-4-yl)amino)methyl)phenyl)-N-hydroxyacrylamide formate was prepared according to the synthetic method of Example 34, and the structure and characterization data are as follows:

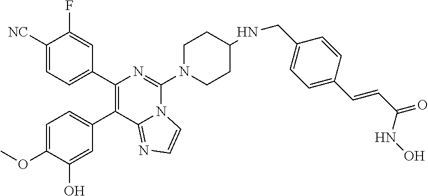 [SMILES: COc1ccc(-c2c(-c3ccc(C#N)c(F)c3)nc(N3CCC(NCc4ccc(/C=C/C(=O)NO)cc4)CC3)n3ccnc23)cc1O]

¹H NMR (400 MHZ, DMSO-d₆) δ 10.72 (s, 1H), 9.04 (s, 1H), 7.85 (s, 1H), 7.80 (t, J=7.6 Hz, 1H), 7.64 (s, 1H), 7.57-7.39 (m, 6H), 7.35-7.28 (m, 1H), 6.89 (d, J=8.4 Hz, 1H), 6.83 (d, J=2.0 Hz, 1H), 6.65 (dd, J=8.2, 2.0 Hz, 1H), 6.44 (d, J=15.8 Hz, 1H), 3.90 (d, J=12.0 Hz, 2H), 3.84 (s, 2H), 3.79 (s, 3H), 3.08 (t, J=11.2 Hz, 2H), 2.78 (s, 1H), 2.14-1.93 (m, 2H), 1.62 (q, J=5.0, 5.6 Hz, 2H).

ESI-MS m/z=634.2 [M+H]⁺.

### Example 71

### Preparation of (E)-3-(4-(((1-(4-(4-cyano-3-fluorophenyl)-5-(3-hydroxy-4-methoxyphenyl)pyrimidin-2-yl)piperidin-4-yl)amino)methyl)-N-hydroxyacrylamide formate

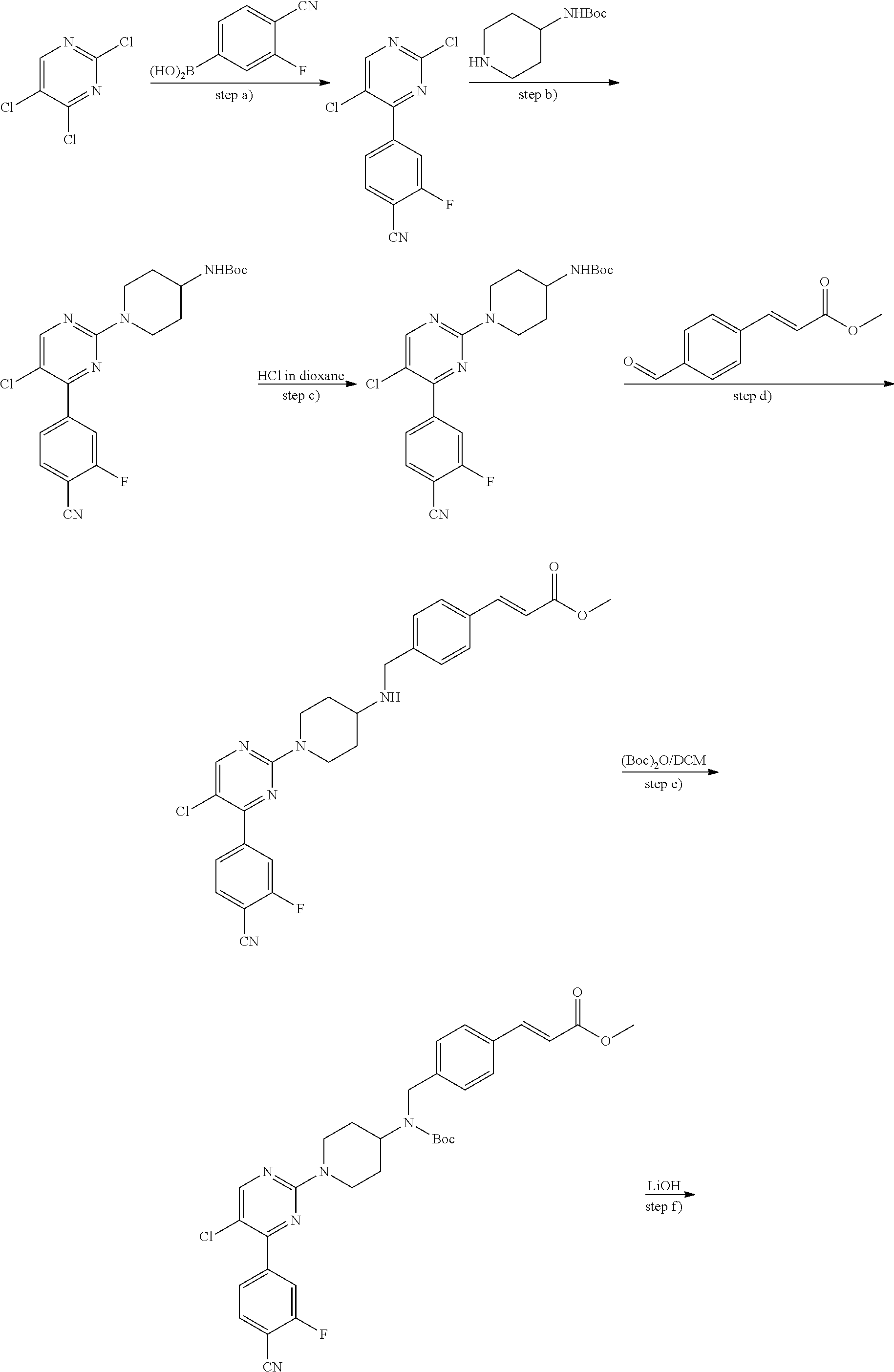

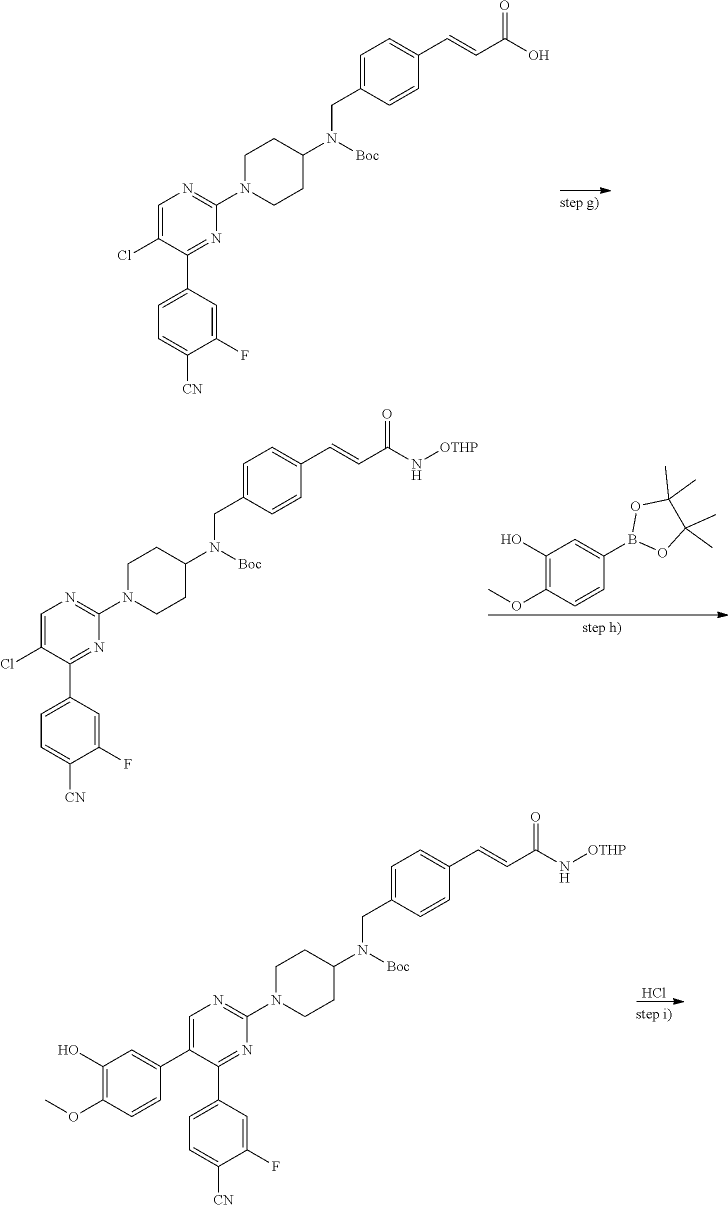

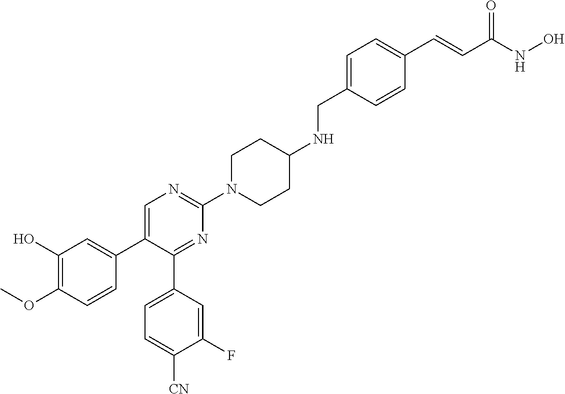 [SMILES: COc1ccc(-c2cnc(N3CCC(NCc4ccc(/C=C/C(=O)NO)cc4)CC3)nc2-c2ccc(C#N)c(F)c2)cc1O]

### Step a): Synthesis of 4-(2,5-dichloropyrimidin-4-yl)-2-fluorobenzonitrile

2,4,5-Trichloropyrimidine (4.0 g, 21.8 mmol), (4-cyano-3-fluorophenyl)boronic acid (3.59 g, 21.8 mmol), cesium carbonate (14.2 g, 43.6 mmol), and [1,1′-bis(diphenylphosphino)ferrocene]palladium dichloride (0.8 g, 0.81 mmol) were dissolved in 1,4-dioxane (60 mL) and water (15 mL) solution. The reaction was run in four parallel vessels and subjected to microwave heating at 110° C. for 45 minutes under nitrogen. After cooling to room temperature, water (40 mL) was added to dilute the reactionsolution, which was then extracted with ethyl acetate (30 mL×3). The organic phases were combined, washed with saturated brine (20 mL), dried over anhydrous sodium sulfate, filtered, and concentrated to dryness under reduced pressure. The residue was purified by silica gel column chromatography (eluent: petroleum ether:ethyl acetate=4:1) to afford 4-(2,5-dichloropyrimidin-4-yl)-2-fluorobenzonitrile with a yield of 53.5%.

ESI-MS m/z: 268.0 [M+H]⁺.

### Step b): synthesis of tert-butyl (1-(5-chloro-4-(4-cyano-3-fluorophenyl)pyrimidin-2-yl)piperidin-4-yl)carbamate

4-(2,5-Dichloropyrimidin-4-yl)-2-fluorobenzonitrile (3.5 g, 13.1 mmol) and tert-butyl piperidin-4-yl-carbamate (2.62 g, 13.1 mmol) were dissolved in DMF (50 mL), then DIPEA (5.1 g, 39.6 mmol) was added to the solution, and the mixture was refluxed at 120° C. for 2 hours under nitrogen. After cooling to room temperature, water (50 mL) was added to dilute the solution, followed by extraction with ethyl acetate (50 mL×3), and the organic phases were combined, washed with saturated brine (50 mL×2), dried over anhydrous sodium sulfate, filtered, and concentrated to dryness under reduced pressure. The residue was purified by silica gel column chromatography (eluent: petroleum ether:ethyl acetate=2:1) to afford tert-butyl (1-(5-chloro-4-(4-cyano-3-fluorophenyl)pyrimidin-2-yl)piperidin-4-yl)carbamate with a yield of 96.2%.

¹H NMR (400 MHZ, DMSO-d₆) δ ppm 8.55 (s, 1H), 8.09 (dd, J=8.2, 6.8 Hz, 1H), 7.89 (dd, J=10.2, 1.6 Hz, 1H), 7.78 (dd, J=8.2, 1.6 Hz, 1H), 6.86 (d, J=7.8 Hz, 1H), 4.57-4.41 (m, 2H), 3.55 (s, 1H), 3.15-2.99 (m, 2H), 1.79 (dd, J=13.4, 3.9 Hz, 2H), 1.38 (s, 9H), 1.35-1.26 (m, 2H).

ESI-MS m/z: 432.2 [M+H]⁺.

### Step c): synthesis of 4-(2-(4-aminopiperidin-1-yl)-5-chloropyrimidin-4-yl)-2-fluorobenzonitrile

Tert-butyl (1-(5-chloro-4-(4-cyano-3-fluorophenyl)pyrimidin-2-yl)piperidin-4-yl)carbamate (2.1 g, 4.87 mmol) was dissolved in 4M hydrochloric acid solution in ethyl acetate (5 mL), and the mixture was stirred at room temperature for 30 minutes under nitrogen, After the reaction was completed as indicated by LCMS, the mixture was concentrated under reduced pressure to afford 4-(2-(4-aminopiperidin-1-yl)-5-chloropyrimidin-4-yl)-2-fluorobenzonitrile with a yield of 95%.

ESI-MS m/z: 332.1 [M+H]⁺.

### Step d): synthesis of methyl (E)-3-(4-(((1-(5-chloro-4-(4-cyano-3-fluorophenyl)pyrimidin-2-yl)piperidin-4-yl)amino)methyl)phenyl)acrylate

4-(2-(4-Aminopiperidin-1-yl)-5-chloropyrimidin-4-yl)-2-fluorobenzonitrile (1.7 g, 3.94 mmol) and methyl (E)-3-(4-formylphenyl)acrylate (1.1 g, 5.91 mmol) were dissolved in 1,2-dichloroethane (20 mL), then acetic acid (2.4 g, 39.4 mmol) and methanol (6.3 g, 197 mmol) were added, and the mixture was stirred for 3 hours under nitrogen. Sodium cyanoborohydride (1.2 g, 19.7 mmol) was added, and stirred for 16 hours. The reaction solution was concentrated to dryness under reduced pressure, and the residue was purified by silica gel column chromatography (eluent: dichloromethane:methanol=10:1) to afford methyl (E)-3-(4-(((1-(5-chloro-4-(4-cyano-3-fluorophenyl)pyrimidin-2-yl)piperidin-4-yl)amino)methyl)phenyl)acrylate with a yield of 11.0%.

ESI-MS m/z: 506.2 [M+H]⁺.

### Step e): synthesis of methyl (E)-3-(4-(((tert-butoxycarbonyl)(1-(5-chloro-4-(4-cyano-3-fluorophenyl)pyrimidin-2-yl)piperidin-4-yl)amino)methyl)phenyl)acrylate

Methyl (E)-3-(4-(((1-(5-chloro-4-(4-cyano-3-fluorophenyl)pyrimidin-2-yl)piperidin-4-yl)amino)methyl)phenyl)acrylate (250 mg, 0.49 mmol), di-tert-butyl dicarbonate (214 mg, 0.98 mmol), and triethylamine (150 mg, 1.44 mmol) were dissolved in dry dichloromethane (10 mL), and the mixture was stirred at room temperature. After the reaction was completed as indicated by LCMS, the solution was concentrated to dryness under reduced pressure. The residue was purified by silica gel column chromatography (eluent: petroleum ether:ethyl acetate=1:2) to afford methyl (E)-3-(4-(((tert-butoxycarbonyl)(1-(5-chloro-4-(4-cyano-3-fluorophenyl)pyrimidin-2-yl)piperidin-4-yl)amino)methyl)phenyl)acrylate with a yield of 90.9%. ¹H NMR (400 MHZ, DMSO-d₆) δ ppm 8.53 (s, 1H), 8.07 (dd, J=8.2, 6.8 Hz, 1H), 7.85 (dd, J=10.2, 1.6 Hz, 1H), 7.76 (dd, J=8.2, 1.6 Hz, 1H), 7.69-7.58 (m, 3H), 7.25 (d, J=7.8 Hz, 2H), 6.58 (d, J=16.0 Hz, 1H), 4.67 (d, J=13.2 Hz, 2H), 4.37 (s, 2H), 4.17 (s, 1H), 3.72 (s, 3H), 2.90 (s, 2H), 1.63 (d, J=8.4 Hz, 4H), 1.41-1.28 (m, 9H).

ESI-MS m/z: 592.2 [M+H]⁺.

### Step f): synthesis of (E)-3-(4-(((tert-butoxycarbonyl)(1-(5-chloro-4-(4-cyano-3-fluorophenyl)pyrimidin-2-yl)piperidin-4-yl)amino)methyl)phenyl)acrylic acid

Methyl (E)-3-(4-(((tert-butoxycarbonyl)(1-(5-chloro-4-(4-cyano-3-fluorophenyl)pyrimidin-2-yl)piperidin-4-yl)amino)methyl)phenyl)acrylate (250 mg, 0.41 mmol) and lithium hydroxide monohydrate (168.2 mg, 4.1 mmol) were dissolved in tetrahydrofuran (THF) (3 mL) and water (3 mL) solution, and the mixture was stirred for 16 hours at room temperature under nitrogen, acidified by adding dilute hydrochloric acid (0.5 mL), followed by extraction with ethyl acetate (10 mL×3). The organic phases were combined and washed with saturated brine (10 mL), dried over anhydrous sodium sulfate, filtered, and concentrated to dryness under reduced pressure to afford (E)-3-(4-(((tert-butoxycarbonyl)(1-(5-chloro-4-(4-cyano-3-fluorophenyl)pyrimidin-2-yl)piperidin-4-yl)amino)methyl)phenyl)acrylic acid with a yield of 90.0%.

ESI-MS m/z: 592.2 [M+H]⁺.

### Step g): synthesis of tert-butyl (E)-(1-(5-chloro-4-(4-cyano-3-fluorophenyl)pyrimidin-2-yl)piperidin-4-yl)(4-(3-oxo-3-(((tetrahydro-2H-pyran-2-yl)oxy)amino)prop-1-en-1-yl)benzyl)carbamate

(E)-3-(4-(((Tert-butoxycarbonyl)(1-(5-chloro-4-(4-cyano-3-fluorophenyl)pyrimidin-2-yl)piperidin-4-yl)amino)methyl)phenyl)acrylic acid (260 mg, 0.44 mmol) and O-(tetrahydro-2H-pyran-2-yl)hydroxylamine (206 mg, 1.76 mmol) were dissolved in DMF (5 mL), and the mixture was stirred for 20 minutes under nitrogen, then 2-(7-azobenzotriazolyl)-N,N,N′,N′-tetramethyluronium hexafluorophosphate (251 mg, 0.66 mmol) and DIPEA (284.3 mg, 2.2 mmol) were added, and the mixture was stirred for 16 hours under nitrogen. After the reaction was completed as indicated by LCMS, water (10 mL) was added, followed by extraction with ethyl acetate (10 mL×3). And the organic phases were combined, washed with saturated brine (10 mL×2), dried over anhydrous sodium sulfate, filtered, and concentrated to dryness under reduced pressure. The residue was purified by silica gel column chromatography (eluent: petroleum ether:ethyl acetate=1:1) to afford tert-butyl (E)-(1-(5-chloro-4-(4-cyano-3-fluorophenyl)pyrimidin-2-yl)piperidin-4-yl)(4-(3-oxo-3-(((tetrahydro-2H-pyran-2-yl)oxy)amino)prop-1-en-1-yl)benzyl)carbamate with a yield of 77.9%.

ESI-MS m/z: 691.3 [M+H]⁺.

### Step h): synthesis of tert-butyl (E)-(1-(4-(4-cyano-3-fluorophenyl)-5-(3-hydroxy-4-methoxyphenyl)pyrimidin-2-yl)piperidin-4-yl)(4-(3-oxo-3-(((tetrahydro-2H-pyran-2-yl)oxy)amino)prop-1-en-1-yl)benzyl)carbamate

Tert-butyl (E)-(1-(5-chloro-4-(4-cyano-3-fluorophenyl)pyrimidin-2-yl)piperidin-4-yl)(4-(3-oxo-3-(((tetrahydro-2H-pyran-2-yl)oxy)amino)prop-1-en-1-yl)benzyl)carbamate (150 mg, 0.22 mmol), 2-methoxy-5-(4,4,5,5-tetramethyl-1,3,2-dioxaborolan-2-yl)phenol (137.6 mg, 0.55 mmol), cesium carbonate (215 mg, 0.66 mmol), palladium acetate (9.8 mg, 0.04 mmol), and bis(di-tert-butyl (4-dimethylaminophenyl)phosphine) dichloropalladium (II) (31.2 mg, 0.04 mmol) were dissolved in a mixture of 1,4-dioxane (5 mL) and water (1 mL), and the mixture was subjected to microwave heating at 120° C. and reacted for 3 hours under nitrogen. After cooling to room temperature, the solution was concentrated under reduced pressure. Preparative thin-layer chromatography on silica gel purification (developing agent: dichloromethane:methanol=12:1) afforded tert-butyl (E)-(1-(4-(4-cyano-3-fluorophenyl)-5-(3-hydroxy-4-methoxyphenyl)pyrimidin-2-yl)piperidin-4-yl)(4-(3-oxo-3-(((tetrahydro-2H-pyran-2-yl)oxy)amino)prop-1-en-1-yl)benzyl)carbamate with a yield of 42.8%.

ESI-MS m/z: 779.3 [M+H]⁺.

### Step i): synthesis of (E)-3-(4-(((1-(4-(4-cyano-3-fluorophenyl)-5-(3-hydroxy-4-methoxyphenyl)pyrimidin-2-yl)piperidin-4-yl)amino)methyl)-N-hydroxyacrylamide formate

Tert-butyl (E)-(1-(4-(4-cyano-3-fluorophenyl)-5-(3-hydroxy-4-methoxyphenyl)pyrimidin-2-yl)piperidin-4-yl)(4-(3-oxo-3-(((tetrahydro-2H-pyran-2-yl)oxy)amino)prop-1-en-1-yl)benzyl)carbamate (64 mg, 0.08 mmol) was dissolved in 4 M hydrochloric acid solution in ethyl acetate (2 mL), and the mixture was stirred for 30 minutes under nitrogen, then concentrated at low temperature to dryness. The residue was purified by Pre-HPLC (separation method 3) to afford (E)-3-(4-(((1-(4-(4-cyano-3-fluorophenyl)-5-(3-hydroxy-4-methoxyphenyl)pyrimidin-2-yl)piperidin-4-yl)amino)methyl)-N-hydroxyacrylamide formate, with a yield of 17.0%. ¹H NMR (400 MHZ, Methanol-d₄) δ ppm 8.52 (s, 2H), 8.38 (s, 1H), 7.69-7.57 (m, 3H), 7.51-7.40 (m, 3H), 7.35 (dd, J=8.2, 1.6 Hz, 1H), 6.95-6.87 (m, 1H), 6.62-6.55 (m, 2H), 6.50 (d, J=15.8 Hz, 1H), 4.94 (d, J=13.6 Hz, 2H), 4.09 (s, 2H), 3.86 (s, 3H), 3.18 (s, 1H), 3.09-2.98 (m, 2H), 2.16 (d, J=11.8 Hz, 2H), 1.53 (qd, J=12.0, 4.2 Hz, 2H).

ESI-MS m/z: 595.2 [M+H]⁺.

### Example 72

### Preparation of (S,E)-3-(4-(((1-(4″-cyano-3″-fluoro-3-hydroxy-4-methoxy-[1,1′:2′,1″-terphenyl]-4′-carbonylpyrrolidin-3-yl)amino)methyl)phenyl)-N-hydroxyacrylamide formate

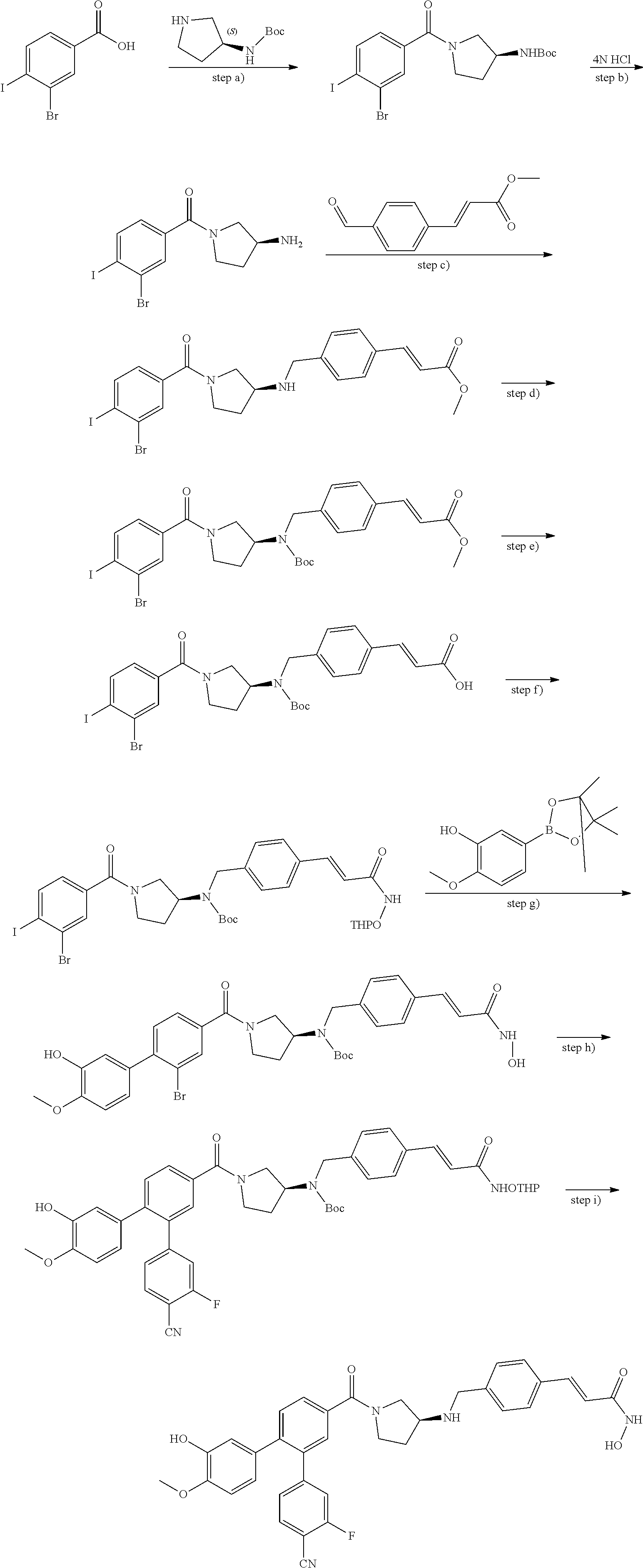

### Step a): Preparation of tert-butyl(S)-(1-(3-bromo-4-iodobenzoyl)pyrrolidin-3-yl)carbamate

3-Bromo-4-iodobenzoic acid (500 mg, 1.5 mmol), tert-butyl(S)-pyrrolidin-3-ylcarbamate (335 mg, 1.8 mmol), DIEA (387 mg, 3.0 mmol), and DMF (5 mL) were added to a reaction flask, then HATU (684 mg, 1.8 mol) was added under stirring at room temperature, and the reaction was maintained for 1 hour at room temperature. After the reaction was completed, it was quenched with water (10 mL), then extracted with ethyl acetate (20 mL×2).And the organic phases were combined, washed sequentially with saturated sodium bicarbonate aqueous solution (20 mL×1) and saturated brine (10 mL×2), dried over anhydrous sodium sulfate, filtered, and the filtrate was concentrated under reduced pressure. The residue was purified by silica gel chromatography (eluent: petroleum ether/ethyl acetate=1/1) to afford tert-butyl(S)-(1-(3-bromo-4-iodobenzoyl)pyrrolidin-3-yl)carbamate with a yield of 60.5%.

ESI-MS (m/z)=495.0 [M+H]⁺.

### Step b): preparation of (S)-(3-aminopyrrolidin-1-yl)(3-bromo-4-iodophenyl)methanone

Tert-butyl(S)-(1-(3-bromo-4-iodobenzoyl)pyrrolidin-3-yl)carbamate (448 mg, 908 μmol) was added to a reaction flask, followed by hydrogen chloride solution in ethyl acetate (4M, 2.5 mL), and the mixture was stirred at room temperature for 1 hour, resulting in the precipitation of a significant amount of solid, then the mixture was concentrated under reduced pressure to afford(S)-(3-aminopyrrolidin-1-yl)(3-bromo-4-iodophenyl)methanone hydrochloride with a yield of 95.5%.

ESI-MS (m/z)=395.0 [M+H]⁺.

### Step c): preparation of methyl (S,E)-3-(4-(1-(3-bromo-4-iodobenzoyl)pyrrolidin-3-yl)amino)methyl)phenyl)acrylate

(S)-(3-Aminopyrrolidin-1-yl)(3-bromo-4-iodophenyl)methanone hydrochloride (343 mg, 867 mmol) and methyl (E)-3-(4-formylphenyl)acrylate (165 mg, 867 mmol) were added to a reaction flask containing DCE (10 mL), and the mixture was stirred at room temperature for 2 hours until the starting materials were completely consumed as indicated by LC-MS. Then sodium cyanoborohydride (0.32 g, 5.04 mmol) was added to the mixture in an ice bath, and stirred at room temperature for 2 hours. The reaction solution was quenched by adding an ice-water cold saturated sodium bicarbonate, followed by extraction with ethyl acetate (20 mL×2). The organic phases were combined, washed with saturated brine (15 mL×2), dried over anhydrous sodium sulfate, filtered, and the residue was purified by silica gel chromatography (eluent: petroleum ether/ethyl acetate=1/1) to afford methyl (S,E)-3-(4-(1-(3-bromo-4-iodobenzoyl)pyrrolidin-3-yl)amino)methyl)phenyl)acrylate with a yield of 72%.

ESI-MS m/z=568.3 [M+H]⁺.

### Step d): preparation of methyl (S,E)-3-(4-(((1-(3-bromo-iodophenyl)pyrrolidin-3-yl)(tert-butoxycarbonyl)aminomethyl)phenyl)acrylate

Methyl (S,E)-3-(4-(1-(3-bromo-4-iodobenzoyl)pyrrolidin-3-yl)amino)methyl)phenyl)acrylate (355 mg, 624 μmol) and triethylamine (1 mL) were dissolved in DCM (5 mL), then di-tert-butyl dicarbonate (150 mg, 936 μmol) was added, and the solution was stirred at room temperature for 1 hour. After the reaction was completed, the reaction solution was concentrated to dryness, and the residue was purified by silica gel chromatography (eluent: petroleum ether/ethyl acetate=1/1) to afford methyl (S,E)-3-(4-((1-(3-bromo-4-iodophenyl)pyrrolidin-3-yl)(tert-butoxycarbonyl)amino)methyl)phenyl)acrylate with a yield of 88%.

ESI-MS m/z=669.1 [M+H]⁺.

### Step e): preparation of (S,E)-3-(4-(1-(3-bromo-4-iodobenzoyl)pyrrolidin-3-yl)(tert-butoxycarbonyl)amino)methyl)phenyl)acrylic acid

Methyl (S,E)-3-(4-((1-(3-bromo-4-iodobenzoyl)pyrrolidin-3-yl)(tert-butoxycarbonyl)amino)methyl)phenyl)acrylate (367 mg, 549 μmol), lithium hydroxide monohydrate (109 mg, 2.6 mmol), THF (2 mL), isopropanol (2 mL), and water (1 mL) were added to a reaction flask and stirred at room temperature for 12 hours. 1N hydrochloric acid was added dropwise with stirring in an ice bath to adjust the pH to 3-4, then water (10 mL) was added, and the mixture was extracted with ethyl acetate (10 mL×3). The organic phases were combined, washed with saturated brine (10 mL×2), and then concentrated to dryness under reduced pressure to afford (S,E)-3-(4-(1-(3-bromo-4-iodobenzoyl)pyrrolidin-3-yl)(tert-butoxycarbonyl)amino)methyl)phenyl)acrylic acid, which was directly used in the next reaction step.

ESI-MS (m/z)=655.2 [M+H]⁺.

### Step f): preparation of tert-butyl ((S)-1-(3-bromo-4-iodobenzoyl)pyrrolidin-3-yl)(4-(E)-3-oxo-3-(tetrahydro-2H-pyran-2-yl)oxy)amino)prop-1-en-1-yl)benzyl)carbamate

(S,E)-3-(4-(1-(3-Bromo-4-iodobenzoyl)pyrrolidin-3-yl)(tert-butoxycarbonyl)amino)methyl)phenyl)acrylic acid (359 mg, 549 μmol), O-(tetrahydro-2H-pyran-2-yl)hydroxylamine (96 mg, 824 μmol), DIEA (142 mg, 1.1 mmol), and DMF (4 mL) were added to a reaction flask, then HATU (250 mg, 659 μmol) was added under stirring at room temperature, and the reaction was continued for 1 hour at room temperature. After the reaction was completed, it was quenched with water (10 mL), then extracted with ethyl acetate (20 mL×2). The organic phases were combined, washed sequentially with saturated sodium bicarbonate aqueous solution (20 mL×1) and saturated brine (10 mL×2), dried over anhydrous sodium sulfate, filtered, and the filtrate was concentrated under reduced pressure. The residue was purified by silica gel chromatography (eluent: petroleum ether/ethyl acetate=1/1) to afford tert-butyl ((S)-1-(3-bromo-4-iodobenzoyl)pyrrolidin-3-yl)(4-(E)-3-oxo-3-(tetrahydro-2H-pyran-2-yl)oxy)amino)prop-1-en-1-yl)benzyl)carbamate with a yield of 60.5%.

ESI-MS (m/z)=754.2 [M+H]⁺.

### g): preparation Step of tert-butyl (S,E)-(1-(2-bromo-3′-hydroxy-4′-methoxy-[1,1′-biphenyl]-4-carbonyl)pyrrolidin-3-yl)(4-(3-(hydroxyamino)-3-oxoprop-1-en-1-yl)benzyl)carbamate

Tert-butyl ((S)-1-(3-bromo-4-iodobenzoyl)pyrrolidin-3-yl)(4-(E)-3-oxo-3-(tetrahydro-2H-pyran-2-yl)oxy)amino)prop-1-en-1-yl)benzyl)carbamate (250 mg, 332 μmol), 2-methoxy-5-(4,4,5,5-tetramethyl-1,3,2-dioxaborolan-2-yl)phenol (100 mg, 398 μmol), Cs₂CO₃ (217 mg, 664 μmol), Pd(dppf)Cl₂ (23 mg, 33 μmol), 1,4-dioxane (4 mL), and H₂O (1 mL) were added to a reaction flask, and the mixture was stirred at 80° C. for 1 hour. The mixture was concentrated to dryness under reduced pressure, and the residue was purified by silica gel chromatography (eluent: petroleum ether/ethyl acetate=2/1) to afford tert-butyl (S,E)-(1-(2-bromo-3′-hydroxy-4′-methoxy-[1,1′-biphenyl]-4-carbonyl)pyrrolidin-3-yl)(4-(3-(hydroxyamino)-3-oxoprop-1-en-1-yl)benzyl)carbamate with a yield of 68.4%.

ESI-MS (m/z)=666.2 [M+H]⁺.

### Step h): preparation of tert-butyl ((S)-1-(4″-cyano-3″-fluoro-3-hydroxy-4-methoxy-[1,1′:2′,1″-terphenyl]-4′-carbonyl)pyrrolidin-3-yl)(4-((E)-3-oxo-3-((tetrahydro-2H-pyran-2-yl)oxy)amino)prop-1-en-1-yl)benzyl)carbamate

Tert-butyl (S,E)-(1-(2-bromo-3′-hydroxy-4′-methoxy-[1,1′-biphenyl]-4-carbonyl)pyrrolidin-3-yl)(4-(3-(hydroxyamino)-3-oxoprop-1-en-1-yl)benzyl)carbamate (151 mg, 227 μmol), (4-cyano-3-fluorophenyl)boronic acid (45 mg, 272 μmol), Cs₂CO₃ (148 mg, 454 μmol), Pd(dppf)Cl₂ (16 mg, 23 μmol), 1,4-dioxane (4 mL), and H₂O (1 mL) were added to a reaction flask, and the mixture was stirred at 90° C. for 1 hour. the mixture was concentrated to dryness under reduced pressure, and the residue was purified by silica gel chromatography (eluent: petroleum ether/ethyl acetate=2/1) to afford tert-butyl ((S)-1-(4″-cyano-3″-fluoro-3-hydroxy-4-methoxy-[1,1′:2′,1″-terphenyl]-4′-carbonyl)pyrrolidin-3-yl)(4-((E)-3-oxo-3-((tetrahydro-2H-pyran-2-yl)oxy)amino)prop-1-en-1-yl)benzyl)carbamate with a yield of 78.5%.

ESI-MS (m/z)=791.1 [M+H]⁺.

### Step i): preparation of (S,E)-3-(4-((1-(4″-cyano-3″-fluoro-3-hydroxy-4-methoxy-[1,1]: 2′,1″-terphenyl]-4′-carbonyl)pyrrolidin-3-yl)amino)methyl)phenyl)-N-hydroxyacrylamide formate

Tert-butyl ((S)-1-(4″-cyano-3″-fluoro-3-hydroxy-4-methoxy-[1,1:2′,1″-terphenyl]-4′-carbonyl)pyrrolidin-3-yl)(4-((E)-3-oxo-3-((tetrahydro-2H-pyran-2-yl)oxy)amino)prop-1-en-1-yl)benzyl)carbamate (141 mg, 178 μmol) was added to a reaction flask, followed by hydrogen chloride solution in ethyl acetate (4M,2.5 mL), and the mixture was stirred at room temperature for 1 hour, resulting in the precipitation of a significant amount of solid. After concentrating under reduced pressure, the crude product was purified by Prep-HPLC (separation method 3) to afford (S,E)-3-(4-((1-(4″-cyano-3″-fluoro-3-hydroxy-4-methoxy-[1,1]: 2′,1″-terphenyl]-4′-carbonyl)pyrrolidin-3-yl)amino)methyl)phenyl)-N-hydroxyacrylamide formate with a yield of 18.5%.

¹H NMR (400 MHZ, DMSO-d₆) δ 7.77-7.40 (m, 8H), 7.37 (d, J=7.4 Hz, 1H), 7.16 (dd, J=4.8, 8.8 Hz, 2H), 6.90-6.79 (m, 1H), 6.58 (s, 2H), 6.46 (t, J=6.0 Hz, 1H), 3.96 (s, 1H), 3.88-3.80 (m, 6H), 3.61 (s, 2H), 3.40-3.31 (m, 1H), 2.54-2.48 (m, 1H), 1.92 (dd, J=7.2, 6.2 Hz, 1H).

ESI-MS (m/z)=607.2 [M+H]⁺.

### Example 73

(S,E)-3-(4-(((1-(4″-Cyano-3″-fluoro-4-hydroxy-3-methoxy-[1,1]: 2′,1″-terphenyl]-4′-carbonyl)pyrrolidin-3-yl)amino)methyl)phenyl)-N-hydroxyacrylamide formate was prepared according to the synthetic method of Example 72, and the structure and characterization data are as follows:

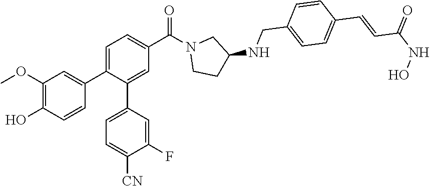 [SMILES: COc1cc(-c2ccc(C(=O)N3CC[C@H](NCc4ccc(/C=C/C(=O)NO)cc4)C3)cc2-c2ccc(C#N)c(F)c2)ccc1O]

¹H NMR (400 MHZ, Methanol-d₄) δ 8.46 (s, 1H), 7.70-7.40 (m, 8H), 7.35 (d, J=7.8 Hz, 1H), 7.14 (dt, J=11.2, 8.8 Hz, 2H), 6.85 (dd, J=8.2, 3.2 Hz, 1H), 6.62-6.53 (m, 2H), 6.45 (t, J=5.8 Hz, 1H), 3.89 (s, 1H), 3.84-3.79 (m, 4H), 3.79-3.36 (m, 5H), 2.34-2.11 (m, 1H), 1.93 (dd, J=13.3, 6.7 Hz, 1H).

ESI-MS m/z=607.3 [M+H]⁺.

### Example 74

### Preparation of (S,E)-3-(4-(((1-((4″-cyano-3″-fluoro-3-hydroxy-4-methoxy-[1,1:2′,1″-terphenyl]-4′-yl)methyl)pyrrolidin-3-yl)amino)methyl)phenyl)-N-hydroxyacrylamide formate

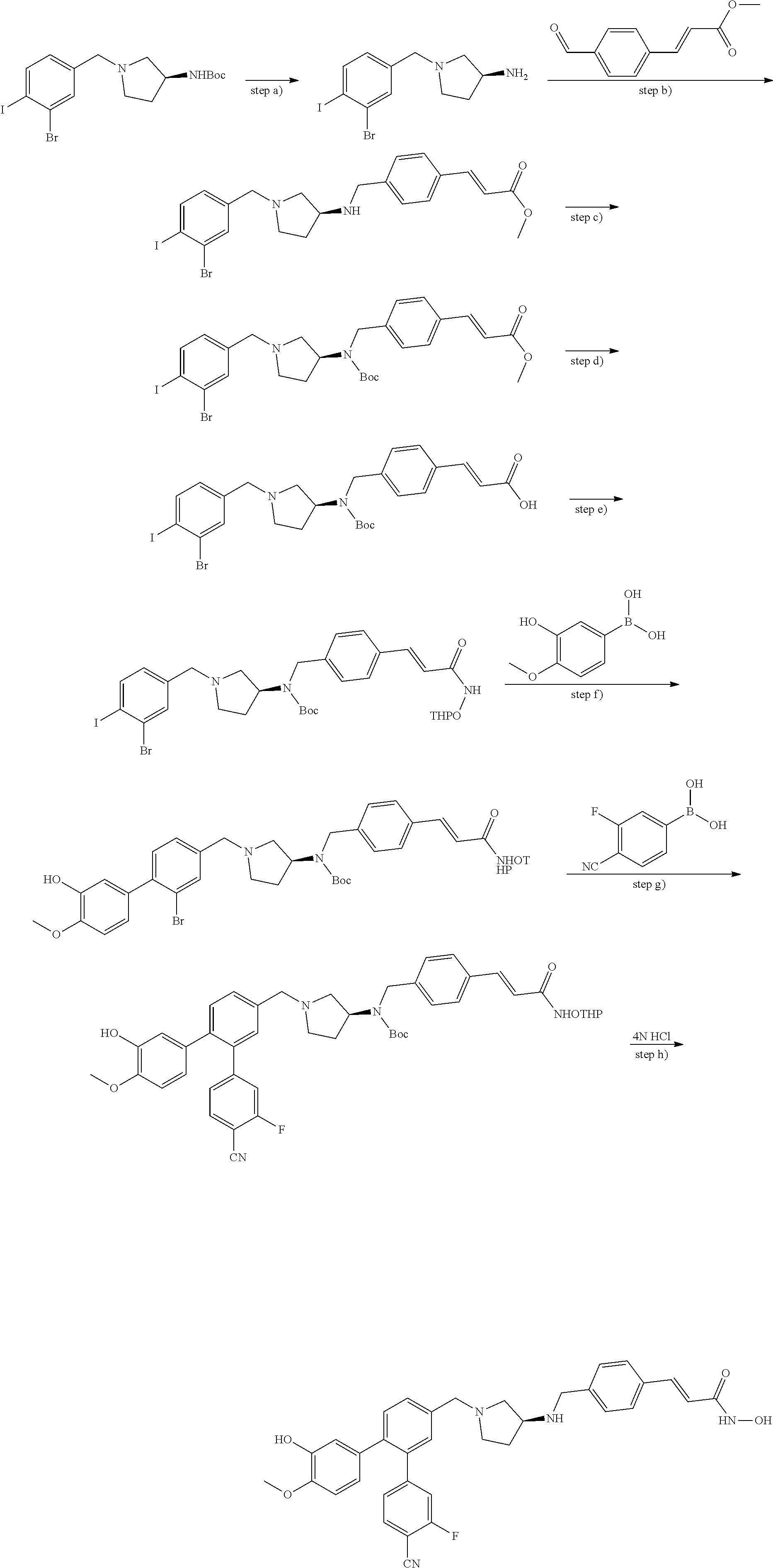

### Step a): Preparation of (S)-1-(3-bromo-4-iodobenzyl)pyrrolidin-3-amine

Tert-butyl(S)-(1-(3-bromo-4-iodobenzyl)pyrrolidin-3-yl)carbamate (500 mg, 1.0 mmol) was added to a reaction flask, followed by hydrogen chloride solution in ethyl acetate (4M, 2.5 mL), and the mixture was stirred at room temperature for one hour, resulting in the precipitation of a significant amount of solid. The mixture was concentrated under reduced pressure to afford(S)-1-(3-bromo-4-iodobenzyl)pyrrolidin-3-amine hydrochloride with a yield of 95.5%.

ESI-MS (m/z)=380.9 [M+H]⁺.

### Step b): preparation of methyl (S,E)-3-(4-(((1-(3-bromo-4-iodobenzyl)pyrrolidin-3-yl)amino)methyl)phenyl)acrylate

(S)-1-(3-bromo-4-iodobenzyl)pyrrolidin-3-amine hydrochloride (362 mg, 955 mmol) and methyl (E)-3-(4-formylphenyl)acrylate (181 mg, 955 mmol) were added to a reaction flask containing DCE (10 mL), and the mixture was stirred at room temperature for 2 hours until the starting materials were completely consumed as monitored by LC-MS, then sodium cyanoborohydride (0.32 g, 4.8 mmol) was then added to the mixture in an ice bath, and was stirred at room temperature for another 2 hours. The reactionsolution was quenched by adding an ice-water cold saturated sodium bicarbonate, followed by extraction with ethyl acetate (20 mL×2). The organic phases were combined, washed with saturated brine (15 mL×2), dried over anhydrous sodium sulfate, filtered, and the residue was purified by silica gel chromatography (eluent: petroleum ether/ethyl acetate=1/1) to afford methyl (S,E)-3-(4-(((1-(3-bromo-4-iodobenzyl)pyrrolidin-3-yl)amino)methyl)phenyl)acrylate with a yield of 68%.

ESI-MS m/z=555.0 [M+H]⁺.

### Step c): preparation of methyl (S,E)-3-(4-(1-(3-bromo-4-iodobenzyl)pyrrolidin-3-yl)(tert-butoxycarbonyl)amino)methyl)phenyl)acrylate

methyl (S,E)-3-(4-(((1-(3-bromo-4-iodobenzyl)pyrrolidin-3-yl)amino)methyl)phenyl)acrylate (360 mg, 649 μmol) and triethylamine (1 mL) were dissolved in DCM (5 mL), then di-tert-butyl dicarbonate (150 mg, 936 μmol) was added, and the reaction was stirred at room temperature for 1 hour. After the reaction was completed, the reaction solution was concentrated to dryness, and the residue was purified by silica gel chromatography (eluent: petroleum ether/ethyl acetate=1/1) to afford methyl (S,E)-3-(4-(1-(3-bromo-4-iodobenzyl)pyrrolidin-3-yl)(tert-butoxycarbonyl)amino)methyl)phenyl)acrylate with a yield of 91%.

ESI-MS m/z=655.1 [M+H]⁺.

### Step d): preparation of (S,E)-3-(4-(1-(3-bromo-4-iodobenzyl)pyrrolidin-3-yl)(tert-butoxycarbonyl)amino)methyl)phenyl)acrylic acid

Methyl (S,E)-3-(4-(1-(3-bromo-4-iodobenzyl)pyrrolidin-3-yl)(tert-butoxycarbonyl)amino)methyl)phenyl)acrylate (387 mg, 591 μmol), lithium hydroxide monohydrate (124 mg, 3.0 mmol), tetrahydrofuran (2 mL), isopropanol (2 mL), and water (1 mL) were added to a reaction flask and the mixture was stirred at room temperature for 12 hours. The pH was adjusted to pH=3˜4 by slowly the addition of 1N hydrochloric acid under stirring in an ice bath, then water (10 mL) was added. And the mixture was extracted with ethyl acetate (10 mL×3), the organic phases were combined and washed with saturated brine (10 mL×2), concentrated to dryness under reduced pressure to afford (S,E)-3-(4-(1-(3-bromo-4-iodobenzyl)pyrrolidin-3-yl)(tert-butoxycarbonyl)amino)methyl)phenyl)acrylic acid, which was directly used in the next step.

ESI-MS (m/z)=641.1 [M+H]⁺.

### Step e): preparation of tert-butyl ((S)-1-(3-bromo-4-iodobenzyl)pyrrolidin-3-yl)(4-(E)-3-oxo-3-(tetrahydro-2H-pyran-2-yl)oxy)amino)prop-1-en-1-yl)benzyl)carbamate

(S,E)-3-(4-(1-(3-Bromo-4-iodobenzyl)pyrrolidin-3-yl)(tert-butoxycarbonyl)amino)methyl)phenyl)acrylic acid (379 mg, 591 μmol), O-(tetrahydro-2H-pyran-2-yl)hydroxylamine (104 mg, 887 μmol), DIEA (142 mg, 1.2 mmol), and DMF (4 mL) were added to a reaction flask, then HATU (270 mg, 709 μmol) was added under stirring at room temperature, and the reaction was stirred for 1 hour at room temperature. After the reaction was completed, it was quenched with water (10 mL), followed by extraction with ethyl acetate (20 mL×2). The organic phases were combined, washed sequentially with saturated sodium bicarbonate solution (20 mL×1) and saturated brine (10 mL×2), dried over anhydrous sodium sulfate, filtered, and the filtrate was concentrated under reduced pressure. The residue was purified by silica gel chromatography (eluent: petroleum ether/ethyl acetate=1/1) to afford tert-butyl ((S)-1-(3-bromo-4-iodobenzyl)pyrrolidin-3-yl)(4-(E)-3-oxo-3-(tetrahydro-2H-pyran-2-yl)oxy)amino)prop-1-en-1-yl)benzyl)carbamate with a yield of 63.1%.

ESI-MS (m/z)=740.1 [M+H]⁺.

### Step f): preparation of tert-butyl ((S)-1-((2-bromo-3′-hydroxy-4′-methoxy-[1,1′-biphenyl]-4-yl)methyl)pyrrolidin-3-yl)(4-(E)-3-oxo-3-(tetrahydro-2H-pyran-2-yl)oxy)amino)prop-1-en-1-yl)benzyl)carbamate

Tert-butyl ((S)-1-(3-bromo-4-iodophenzyl)pyrrolidin-3-yl)(4-((E)-3-oxo-3-(tetrahydro-2H-pyran-2-yl)oxy)amino)prop-1-en-1-yl)benzyl)carbamate (275 mg, 372 μmol), (3-hydroxy-4-methoxyphenyl)boronic acid (112 mg, 446 μmol), Cs₂CO₃ (243 mg, 744 μmol), Pd(dppf)Cl₂ (27 mg, 37 μmol), 1,4-dioxane (4 mL), and water (H₂O) (1 mL) were added to a reaction flask, and the mixture was stirred at 80° C. for 1 hour. The mixture was concentrated to dryness under reduced pressure, and the residue was then purified by silica gel chromatography (eluent: petroleum ether/ethyl acetate=2/1) to afford tert-butyl ((S)-1-((2-bromo-3′-hydroxy-4′-methoxy-[1,1′-biphenyl]-4-yl)methyl)pyrrolidin-3-yl)(4-(E)-3-oxo-3-(tetrahydro-2H-pyran-2-yl)oxy)amino)prop-1-en-1-yl)benzyl)carbamate with a yield of 70.4%.

ESI-MS (m/z)=736.3 [M+H]⁺.

### Step g): preparation of tert-butyl ((S)-1-((4″-cyano-3″-fluoro-3-hydroxy-4-methoxy-[1,1:2′,1″-terphenyl]-4′-yl)methyl)pyrrolidin-3-yl)(4-((E)-3-oxo-3-((tetrahydro-2H-pyran-2-yl)oxy)amino)prop-1-en-1-yl)benzyl)carbamate

Tert-butyl ((S)-1-((2-bromo-3′-hydroxy-4′-methoxy-[1,1′-biphenyl]-4-yl)methyl)pyrrolidin-3-yl)(4-(E)-3-oxo-3-(tetrahydro-2H-pyran-2-yl)oxy)amino)prop-1-en-1-yl)benzyl)carbamate (192 mg, 262 μmol), (4-cyano-3-fluorophenyl)boronic acid (52 mg, 314 μmol), Cs₂CO₃ (171 mg, 524 μmol), Pd(dppf)Cl₂ (17 mg, 26 μmol), 1,4-dioxane (4 mL), and water (1 mL) were added to a reaction flask, and the mixture was stirred at 90° C. for 1 hour. The mixture was concentrated to dryness under reduced pressure, and the residue was then purified by silica gel chromatography (eluent: petroleum ether/ethyl acetate=2/1) to afford tert-butyl ((S)-1-((4″-cyano-3″-fluoro-3-hydroxy-4-methoxy-[1,1:2′,1″-terphenyl]-4′-yl)methyl)pyrrolidin-3-yl)(4-((E)-3-oxo-3-((tetrahydro-2H-pyran-2-yl)oxy)amino)prop-1-en-1-yl)benzyl)carbamate with a yield of 70.5%.

ESI-MS (m/z)=777.1 [M+H]⁺.

### Step h): preparation of (S,E)-3-(4-(((1-((4″-cyano-3″-fluoro-3-hydroxy-4-methoxy-[1,1:2′,1″-terphenyl]-4′-yl)methyl)pyrrolidin-3-yl)amino)methyl)phenyl)-N-hydroxyacrylamide formate

Tert-butyl ((S)-1-((4″-cyano-3″-fluoro-3-hydroxy-4-methoxy-[1,1:2′,1″-terphenyl]-4′-yl)methyl)pyrrolidin-3-yl)(4-((E)-3-oxo-3-((tetrahydro-2H-pyran-2-yl)oxy)amino)prop-1-en-1-yl)benzyl)carbamate (144 mg, 185 μmol) was added to a reaction flask, followed by hydrogen chloride solution in ethyl acetate (4M, 2.5 mL), and the mixture was stirred at room temperature for 1 hour, resulting in the precipitation of a significant amount of solid. After concentrated under reduced pressure, the crude product was purified by Prep-HPLC (separation method 3) to afford (S,E)-3-(4-((1-((4″-cyano-3″-fluoro-3-hydroxy-4-methoxy-[1,1;2′,1″-terphenyl]-4′-yl)methyl)pyrrolidin-3-yl)amino)methyl)phenyl)-N-hydroxyacrylamide formate with a yield of 15.5%.

¹H NMR (400 MHZ, Methanol-d₄) δ 8.48 (s, 1H), 7.56 (d, J=8.6 Hz, 4H), 7.43-7.03 (m, 5H), 7.13 (s, 2H), 6.83 (d, J=7.8 Hz, 1H), 6.61-6.42 (m, 3H), 3.98 (d, J=7.0 Hz, 4H), 3.83 (s, 3H), 3.69 (s, 1H), 3.14-2.61 (m, 4H), 2.33 (s, 1H), 1.94 (s, 1H).

ESI-MS (m/z)=593.3 [M+H]⁺.

### Example 75

(S,E)-3-(4-(((1-((4″-Cyano-3″-fluoro-4-hydroxy-3-methoxy-[1,1:2,1″-terphenyl]-4′-yl)methyl)pyrrolidin-3-yl)amino)methyl)phenyl)-N-hydroxyacrylamide formate was prepared according to the synthetic method of Example 74, and the structure and characterization data are as follows:

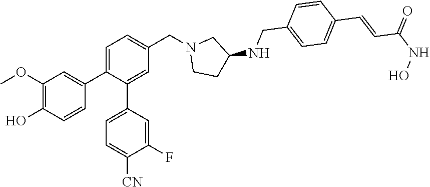 [SMILES: COc1cc(-c2ccc(CN3CC[C@H](NCc4ccc(/C=C/C(=O)NO)cc4)C3)cc2-c2ccc(C#N)c(F)c2)ccc1O]

¹H NMR (400 MHZ, Methanol-d₄) δ 8.40 (s, 1H), 7.74-7.57 (m, 4H), 7.51 (d, J=6.8 Hz, 5H), 7.16 (d, J=3.8 Hz, 2H), 6.73 (d, J=8.0 Hz, 1H), 6.58 (s, 2H), 6.50 (d, J=5.2 Hz, 1H), 4.08 (s, 2H), 3.96 (s, 2H), 3.75 (s, 1H), 3.63 (s, 3H), 3.10 (s, 1H), 2.95 (s, 2H), 2.75 (s, 1H), 2.38-2.30 (m, 4H), 1.97 (dd, J=10.2, 8.0 Hz, 1H).

ESI-MS m/z=593.3 [M+H]⁺.

### Example 76

### Preparation of (E)-3-(4-(((1-(4-(4-cyano-3-fluorophenyl)-3-fluoro-5-(3-hydroxy-4-methoxyphenyl)pyridin-2-yl)piperidin-4-yl)amino)methyl)phenyl)-N-hydroxyacrylamide formate

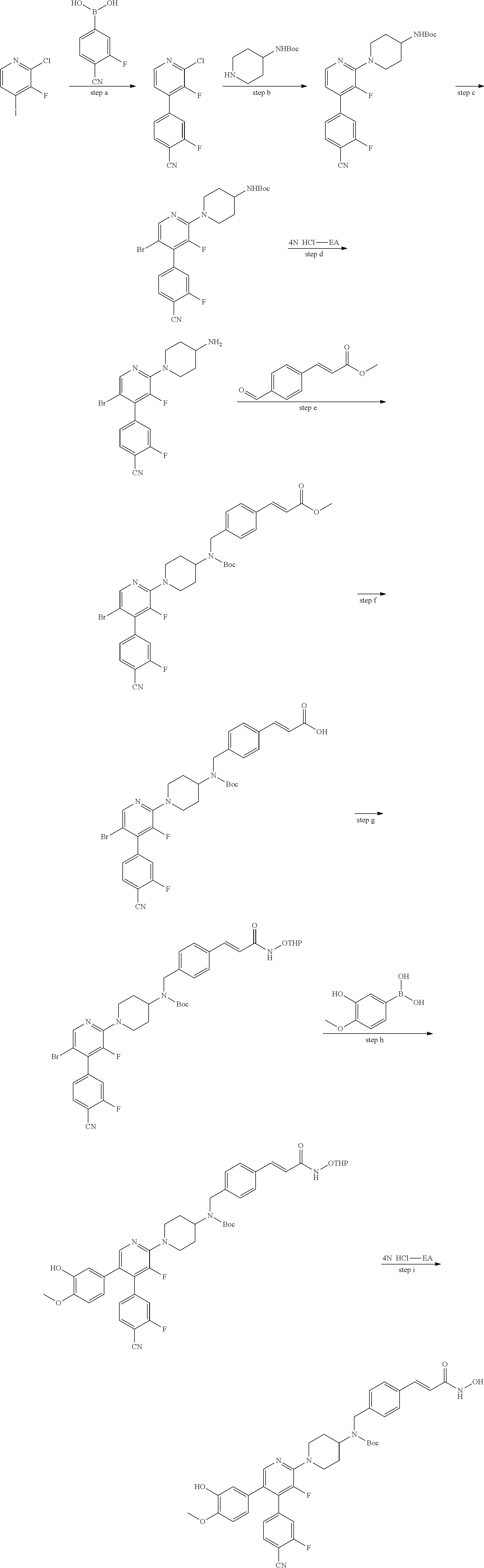

### Step a): preparation of 4-(2-chloro-3-fluoropyridin-4-yl)-2-fluorobenzonitrile

2-Chloro-3-fluoro-4-iodopyridine (2 g, 7.78 mmol), (4-cyano-3-fluorophenyl)boronic acid (1.28 g, 7.78 mmol), CS₂CO₃ (5.07 g, 15.56 mmol), and Pd(dppf)Cl₂ (0.57 g, 0.78 mmol) were dissolved in a mixture of 1,4-dioxane (3 mL) and water (1 mL), and the solution was subjected to microwave heating at 65° C. and reacted for 30 minutes under nitrogen. After concentrated, a crude product was purified by silica gel chromatography (eluent: petroleum ether/ethyl acetate=10/3) to afford 4-(2-chloro-3-fluoropyridin-4-yl)-2-fluorobenzonitrile with a yield of 78%.

ESI-MS m/z=251.0 [M+H]⁺

### Step b): preparation of tert-butyl N-(1-(4-(4-cyano-3-fluorophenyl)-3-fluoropyridin-2-yl)piperidin-4-yl)carbamate

4-(2-Chloro-3-fluoropyridin-4-yl)-2-fluorobenzonitrile (1.0 g, 3.99 mmol), tert-butyl N-(piperidin-4-yl)carbamate (1.1 g, 5.99 mmol), 2-dicyclohexylphosphino-2′,6′-diisopropoxybiphenyl (186.19 mg, 0.40 mmol), tris(dibenzylideneacetone)dipalladium (182.69 mg, 0.20 mmol), and CS₂CO₃ (3.9 g, 11.97 mmol) were dissolved in toluene (15 mL), and the solution was subjected to microwave heating at 110° C. and reacted for 4 hours under nitrogen. Water (200 mL) was added to quench the reaction, followed by extraction with ethyl acetate (200 mL×2). And the organic phases were combined, washed with saturated brine (150 mL×2), dried over anhydrous sodium sulfate, filtered, and concentrated to obtain a crude product. The residue was purified by silica gel chromatography (eluent: petroleum ether/ethyl acetate=5/2) to afford tert-butyl N-(1-(4-(4-cyano-3-fluorophenyl)-3-fluoropyridin-2-yl)piperidin-4-yl)carbamate with a yield of 42%.

ESI-MS m/z=415.2 [M+H]⁺

### Step c): preparation of tert-butyl N-(1-(5-bromo-4-(4-cyano-3-fluorophenyl)-3-fluoropyridin-2-yl)piperidin-4-yl)carbamate

Tert-butyl N-(1-(4-(4-cyano-3-fluorophenyl)-3-fluoropyridin-2-yl)piperidin-4-yl)carbamate (930 mg, 2.24 mmol) and N-bromosuccinimide (398.68 mg, 2.24 mmol) were dissolved in DMF (16 mL) and reacted for 30 minutes. After the reaction was completed, water (20 mL) was added to quench the reaction, followed by extraction with ethyl acetate (20 mL×2). And the organic phases were combined, washed with saturated brine (15 mL×2), dried over anhydrous sodium sulfate, filtered, and concentrated to obtain a crude product. The residue was purified by silica gel chromatography (eluent: petroleum ether/ethyl acetate=10/7) to afford tert-butyl N-(1-(5-bromo-4-(4-cyano-3-fluorophenyl)-3-fluoropyridin-2-yl)piperidin-4-yl)carbamate with a yield of 87%.

ESI-MS m/z=493.1 [M+H]⁺

Step d): preparation of 4-(2-(4-aminopiperidin-1-yl)-5-bromo-3-fluoropyridin-4-yl)-2-fluorobenzonitrile Tert-butyl N-(1-(5-bromo-4-(4-cyano-3-fluorophenyl)-3-fluoropyridin-2-yl)piperidin-4-yl)carbamate (970 mg, 1.97 mmol) was dissolved in a 4N hydrochloric acid ethyl acetate (20 mL) and reacted for 30 minutes. After concentrating, a crude product was obtained, which was directly used in the next reaction step. ESI-MS m/z=393.0 [M+H]⁺

### Step e): preparation of methyl (E)-3-(4-(1-(5-bromo-4-(4-cyano-3-fluorophenyl)-3-fluoropyridin-2-yl)piperidin-4-yl)(tert-butoxycarbonyl)amino)methyl)phenyl)acrylate

4-(2-(4-Aminopiperidin-1-yl)-5-bromo-3-fluoropyridin-4-yl)-2-fluorobenzonitrile (220 mg, 0.51 mmol) and methyl (E)-3-(4-formylphenyl)acrylate (97.00 mg, 0.51 mmol) were dissolved in 1,2-dichloroethane (6 mL), methanol (40 μL), and acetic acid (10 μL) and reacted for 30 minutes. Then the reaction solution was cooled in an ice bath, and sodium cyanoborohydride (96.15 mg, 1.53 mmol) was added and reacted overnight. The reaction solution was quenched with water (5 mL), and the mixture was concentrated to obtain a crude product. Then a solution of di-tert-butyl dicarbonate (320.83 mg, 1.47 mmol) and Na₂CO₃ (155.82 mg, 1.47 mmol) tetrahydrofuran (THF) (3 mL) and water (2 mL) was added to the above crude product to react for 30 minutes. Water (20 mL) was added to quenched the reaction, and the mixture was extracted with ethyl acetate (20 mL×2). The organic phases were combined, washed with saturated brine (15 mL×2), dried over anhydrous sodium sulfate, filtered, and concentrated to obtain a crude product. The residue was purified by silica gel chromatography (eluent: petroleum ether/ethyl acetate=10/7) to afford methyl (E)-3-(4-(1-(5-bromo-4-(4-cyano-3-fluorophenyl)-3-fluoropyridin-2-yl)piperidin-4-yl)(tert-butoxycarbonyl)amino)methyl)phenyl)acrylate with a yield of 91%.

ESI-MS m/z=666.2 [M+H]⁺

### Step f): preparation of (E)-3-(4-(1-(5-bromo-4-(4-cyano-3-fluorophenyl)-3-fluoropyridin-2-yl)piperidin-4-yl)(tert-butoxycarbonyl)amino)methyl)phenyl)acrylic acid

Methyl (E)-3-(4-(1-(5-bromo-4-(4-cyano-3-fluorophenyl)-3-fluoropyridin-2-yl)piperidin-4-yl)(tert-butoxycarbonyl)amino)methyl)phenyl)acrylate (300 mg, 0.45 mmol) and lithium hydroxide monohydrate (188.82 mg, 4.5 mmol) were dissolved in a mixture of THF (3 mL) and water (1 mL) and reacted for 36 hours. After the reaction, water (10 mL) was added, and the pH was adjusted to 3-4 with 1N HCl solution, the mixture was then extracted with ethyl acetate (20 mL×2). And the organic phases were combined and washed with saturated brine (15 mL×2), dried over anhydrous sodium sulfate, filtered, and concentrated to obtain a crude product, which was directly used in the next step.

ESI-MS m/z=653.2 [M+H]⁺

### Step g): preparation of tert-butyl (E)-(1-(5-bromo-4-(4-cyano-3-fluorophenyl)-3-fluoropyridin-2-yl)piperidin-4-yl)(4-(3-oxo-3-(((tetrahydro-2H-pyran-2-yl)oxy)amino)prop-1-en-1-yl)benzyl)carbamate

(E)-3-(4-(1-(5-Bromo-4-(4-cyano-3-fluorophenyl)-3-fluoropyridin-2-yl)piperidin-4-yl)(tert-butoxycarbonyl)amino)methyl)phenyl)acrylic acid (280 mg, 0.43 mmol) and O-(tetrahydro-2H-pyran-2-yl)hydroxylamine (151.12 mg, 1.29 mmol) were dissolved in DMF (30 mL), then HATU (490.51 mg, 1.29 mmol) and DIPEA (277.35 mg, 2.15 mmol) were added, and reacted for 30 minutes. The reaction solution was quenched with water (20 mL), followed by extraction with ethyl acetate (20 mL×2). And the organic phases were combined, washed with saturated brine (15 mL×2), dried over anhydrous sodium sulfate, filtered, and concentrated to obtain a crude product. The residue was then purified by silica gel chromatography (eluent: dichloromethane/methanol=15/1) to afford tert-butyl (E)-(1-(5-bromo-4-(4-cyano-3-fluorophenyl)-3-fluoropyridin-2-yl)piperidin-4-yl)(4-(3-oxo-3-(((tetrahydro-2H-pyran-2-yl)oxy)amino)prop-1-en-1-yl)benzyl)carbamate with a yield of 92%.

ESI-MS m/z=752.2 [M+H]⁺

### Step h): preparation of tert-butyl (E)-(1-(4-(4-cyano-3-fluorophenyl)-3-fluoro-5-(3-hydroxy-4-methoxyphenyl)pyridin-2-yl)piperidin-4-yl)(4-(3-oxo-3-(((tetrahydro-2H-pyran-2-yl)oxy)amino)prop-1-en-1-yl)benzyl)carbamate

Tert-butyl (E)-(1-(5-bromo-4-(4-cyano-3-fluorophenyl)-3-fluoropyridin-2-yl)piperidin-4-yl)(4-(3-oxo-3-(((tetrahydro-2H-pyran-2-yl)oxy)amino)prop-1-en-1-yl)benzyl)carbamate (300 mg, 0.40 mmol), (3-hydroxy-4-methoxyphenyl)boronic acid (100.77 mg, 0.60 mmol), Cs₂CO₃ (390.98 mg, 1.20 mmol), and Pd(dppf)Cl₂ (58.54 mg, 0.080 mmol) were dissolved in a solvent of 1,4-dioxane (3 mL) and water (1 mL), and the mixture was subjected to microwave heating at 90° C. and reacted for 30 minutes under nitrogen. After concentrated, a crude product was obtained, and the residue was then purified by silica gel chromatography (eluent: dichloromethane/methanol=10/1) to afford tert-butyl (E)-(1-(4-(4-cyano-3-fluorophenyl)-3-fluoro-5-(3-hydroxy-4-methoxyphenyl)pyridin-2-yl)piperidin-4-yl)(4-(3-oxo-3-(((tetrahydro-2H-pyran-2-yl)oxy)amino)prop-1-en-1-yl)benzyl)carbamate with a yield of 80%.

ESI-MS m/z=796.3 [M+H]⁺

### Step i): preparation of (E)-3-(4-(4-(4-cyano-3-fluorophenyl)-3-fluoro-5-(3-hydroxy-4-methoxyphenyl)pyridin-2-yl)piperidin-4-yl)amino)methyl)phenyl)-N-hydroxyacrylamide

Tert-butyl tert-butyl (E)-(1-(4-(4-cyano-3-fluorophenyl)-3-fluoro-5-(3-hydroxy-4-methoxyphenyl)pyridin-2-yl)piperidin-4-yl)(4-(3-oxo-3-(((tetrahydro-2H-pyran-2-yl)oxy)amino)prop-1-en-1-yl)benzyl)carbamate (0.7 g, 0.88 mmol) was dissolved in a 4N hydrochloric acid solution in ethyl acetate (15 mL) and reacted for 30 minutes. The mixture was concentrated under reduced pressure at low temperature to obtain a residue, and the residue was purified by Prep-HPLC (separation method 3) to afford (E)-3-(4-(((1-(4-(4-cyano-3-fluorophenyl)-3-fluoro-5-(3-hydroxy-4-methoxyphenyl)pyridin-2-yl)piperidin-4-yl)amino)methyl)phenyl)-N-hydroxyacrylamide formate with a yield of 5%.

¹H NMR (400 MHZ, Methanol-d₄) δ ppm 8.52 (s, 1H), 8.08 (s, 1H), 7.72 (t, J=7.4 Hz, 1H), 7.65 (t, J=8.6 Hz, 2H), 7.62-7.45 (m, 3H), 7.28 (d, J=9.8 Hz, 1H), 7.17 (d, J=8.0 Hz, 1H), 6.84 (d, J=8.8 Hz, 1H), 6.58-6.45 (m, 3H), 4.59 (s, 1H), 4.24 (s, 1H), 4.21 (s, 3H), 3.84 (s, 3H), 3.05 (t, J=12.6 Hz, 2H), 2.24 (d, J=12.2 Hz, 2H), 1.77 (dd, J=13.6, 9.8 Hz, 2H).

ESI-MS m/z=612.2 [M+H]⁺

### Example 77

(E)-3-(4-(((1-(4-(4-Cyano-3-fluorophenyl)-3-fluoro-5-(4-hydroxy-3-methoxyphenyl)pyridin-2-yl)piperidin-4-yl)amino)methyl)phenyl)-N-hydroxyacrylamide formate was prepared according to the synthetic method of Example 76, and the structure and characterization data are as follows:

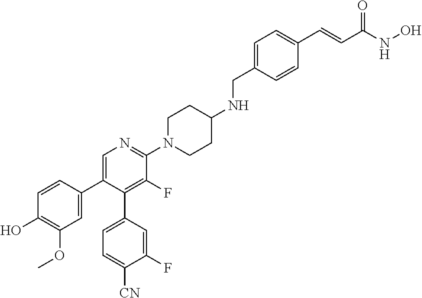 [SMILES: COc1cc(-c2cnc(N3CCC(NCc4ccc(/C=C/C(=O)NO)cc4)CC3)c(F)c2-c2ccc(C#N)c(F)c2)ccc1O]

¹H NMR (400 MHZ, Methanol-d₄) δ ppm 8.52 (s, 2H), 8.12 (s, 1H), 7.73 (dd, J=8.0, 6.8 Hz, 1H), 7.63 (t, J=10.2 Hz, 2H), 7.55 (t, J=8.0 Hz, 3H), 7.30 (d, J=9.8 Hz, 1H), 7.19 (d, J=8.0 Hz, 1H), 6.73 (d, J=8.0 Hz, 1H), 6.64-6.47 (m, 3H), 4.23 (s, 1H), 4.19 (s, 3H), 3.65 (s, 3H), 3.26 (s, 1H), 3.04 (t, J=12.6 Hz, 2H), 2.23 (d, J=12.0 Hz, 2H), 1.78 (q, J=11.2, 10.4 Hz, 2H).

ESI-MS m/z=612.2 [M+H]⁺

### Example 80

### Synthesis of (E)-3-(4-(((1-(4-(4-cyano-3-fluorophenyl)-5-(3-hydroxy-4-methoxyphenyl)pyridin-2-yl)piperidin-4-yl)amino)methyl)phenyl)-N-hydroxyacrylamide formate

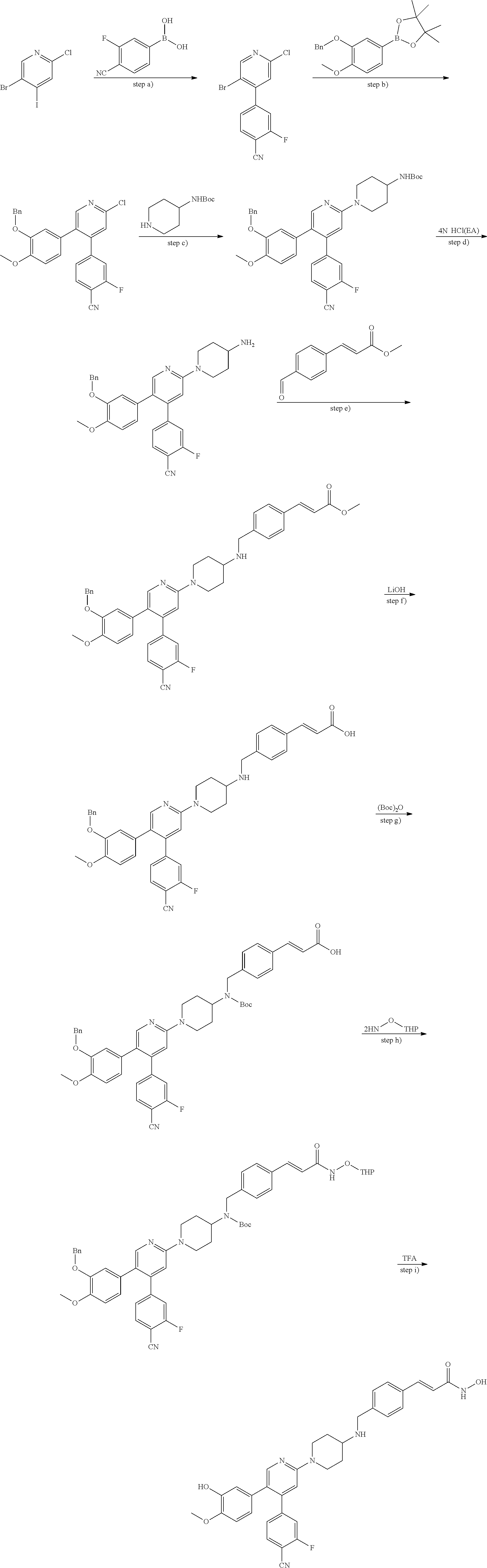

### Step a): synthesis of 4-(5-bromo-2-chloropyridin-4-yl)-2-fluorobenzonitrile

5-Bromo-2-chloro-4-iodopyridine (3.0 g, 9.42 mmol), (4-cyano-3-fluorophenyl)boronic acid (1.5 g, 9.42 mmol), [1,1′-bis(diphenylphosphino)ferrocene]palladium dichloride (689.26 mg, 0.94 mmol), and cesium carbonate (6138.45 mg, 18.84 mmol) were added to a mixture of 1,4-dioxane:water=5:1 (20 mL), and the mixture was subjected to microwave heating at 110° C. and reacted for 1 hour under nitrogen and monitored by LCMS until the starting materials were consumed. The solvent was removed by concentrating, and the residue was mixed with silica gel and purified by column chromatography on silica gel to afford 4-(5-bromo-2-chloropyridin-4-yl)-2-fluorobenzonitrile with a yield of 82.46%.

ESI-MS (m/z)=311.1 [M+H]⁺.

### Step b): synthesis of 4-(5-(3-(benzyloxy)-4-methoxyphenyl)-2-chloropyridin-4-yl)-2-fluorobenzonitrile

4-(5-Bromo-2-chloropyridin-4-yl)-2-fluorobenzonitrile (1.5 g, 4.81 mmol), 2-(3-(benzyloxy)-4-methoxyphenyl)-4,4,5,5-tetramethyl-1,3,2-dioxaborolane (1.9 g, 5.53 mmol), [1,1′-bis(diphenylphosphino)ferrocene]palladium dichloride (351.95 mg, 0.48 mmol), and cesium carbonate (3.1 g, 9.62 mmol) were added to a mixture of 1,4-dioxane:water=5:1 (30 mL), and the mixture was reacted at 80° C. for 1 hour under nitrogen and monitored by LCMS until the starting materials were consumed. The solvent was directly removed by concentrating, and the residue was mixed with silica gel and purified by column chromatography on silica gel to afford 4-(5-(3-(benzyloxy)-4-methoxyphenyl)-2-chloropyridin-4-yl)-2-fluorobenzonitrile with a yield of 88.32%.

ESI-MS (m/z)=445.1 [M+H]⁺.

### Step c): synthesis of tert-butyl (1-(5-(3-(benzyloxy)-4-methoxyphenyl)-4-(4-cyano-3-fluorophenyl)pyridin-2-yl)piperidin-4-yl)carbamate

4-(5-(3-(Benzyloxy)-4-methoxyphenyl)-2-chloropyridin-4-yl)-2-fluorobenzonitrile (1.5 g, 3.3 mmol), tert-butyl N-(piperidin-4-yl)carbamate (990.5 mg, 5.1 mmol), 2-dicyclohexylphosphino-2′,6′-diisopropoxybiphenyl (155 mg, 0.33 mmol), tris(dibenzylideneacetone)dipalladium (150.5 mg, 0.165 mmol), and cesium carbonate (3215.2 mg, 9.9 mmol) were added to toluene (50 mL). The reaction was monitored by LCMS until the starting materials were consumed, and the solvent was removed. The residue was mixed with silica gel and purified by column chromatography on silica gel to afford tert-butyl (1-(5-(3-(benzyloxy)-4-methoxyphenyl)-4-(4-cyano-3-fluorophenyl)pyridin-2-yl)piperidin-4-yl)carbamate with a yield of 51.98%.

ESI-MS (m/z)=609.2 [M+H]⁺.

### Step d): synthesis of 4-(2-(4-aminopiperidin-1-yl)-5-(3-(benzyloxy)-4-methoxyphenyl)pyridin-4-yl)-2-fluorobenzonitrile

tert-butyl (1-(5-(3-(benzyloxy)-4-methoxyphenyl)-4-(4-cyano-3-fluorophenyl)pyridin-2-yl)piperidin-4-yl)carbamate (780 mg, 1.44 mmol) was added to 4N hydrochloric acid in EA (4 mL), and the reaction was stirred at room temperature for 0.5 hours and monitored by LCMS until the starting materials were completely consumed The solvent was removed, and the residue was treated with a saturated solution of sodium bicarbonate to adjust the pH to 8-9. The mixture was extracted with EA, and the organic phases were combined. After the solvent removed, a solid crude product was obtained and was used directly for the next reaction step.

ESI-MS (m/z)=509.3 [M+H]⁺.

### Step e): synthesis of methyl (E)-3-(4-(((1-(5-(3-(benzyloxy)-4-methoxyphenyl)-4-(4-cyano-3-fluorophenyl)pyridin-2-yl)piperidin-4-yl)amino)methyl)phenyl)acrylate

4-(2-(4-Aminopiperidin-1-yl)-5-(3-(benzyloxy)-4-methoxyphenyl)pyridin-4-yl)-2-fluorobenzonitrile (0.35 g, 0.68 mmol), methyl (E)-3-(4-formylphenyl)acrylate (0.16 g, 0.82 mmol), and sodium cyanoborohydride (0.085 g, 1.36 mmol) were added to 1,2-dichloroethane (10 mL), and the mixture was reacted overnight at room temperature and monitored by LCMS until the starting materials were consumed. The solvent was removed, and the residue was mixed with silica gel and purified by column chromatography on silica gel to afford methyl (E)-3-(4-(((1-(5-(3-(benzyloxy)-4-methoxyphenyl)-4-(4-cyano-3-fluorophenyl)pyridin-2-yl)piperidin-4-yl)amino)methyl)phenyl)acrylate with a yield of 75.38%.

ESI-MS (m/z)=683.2 [M+H]⁺.

### Step f): synthesis of (E)-3-(4-(1-(5-(3-(benzyloxy)-4-methoxyphenyl)-4-(4-cyano-3-fluorophenyl)pyridin-2-yl)piperidin-4-amino)methyl)phenyl)acrylic acid

methyl (E)-3-(4-(((1-(5-(3-(benzyloxy)-4-methoxyphenyl)-4-(4-cyano-3-fluorophenyl)pyridin-2-yl)piperidin-4-yl)amino)methyl)phenyl)acrylate (254 mg, 0.37 mmol) and lithium hydroxide (44.31 mg, 1.85 mmol) were added to a mixture of tetrahydrofuran:water=4:1 (5 mL). The solution was stirred overnight at room temperature and monitored by LCMS until the starting materials were consumed, and the crude then was directly used for the next reaction step.

ESI-MS (m/z)=669.2 [M+H]⁺.

### Step g): synthesis of (E)-3-(4-(((1-(5-(3-(benzyloxy)-4-methoxyphenyl)-4-(4-cyano-3-fluorophenyl)pyridin-2-yl)piperidin-4-yl)(tert-butoxycarbonyl)amino)methyl)phenyl)acrylic acid

In the reactionsolution of Step f, di-tert-butyl dicarbonate (170.5 mg, 0.78 mmol) and sodium bicarbonate were added, and the mixture was stirred overnight at room temperature and monitored by LCMS until the starting materials were consumed. The pH was adjusted to weakly acidic with dilute hydrochloric acid, then water was added, and the mixture was extracted with EA. The organic phases were combined, and the solvent was removed. The residue was added with silica gel and purified by column chromatography on silica gel to afford (E)-3-(4-(((1-(5-(3-(benzyloxy)-4-methoxyphenyl)-4-(4-cyano-3-fluorophenyl)pyridin-2-yl)piperidin-4-yl)(tert-butoxycarbonyl)amino)methyl)phenyl)acrylic acid with a yield of 80.64%.

ESI-MS (m/z)=769.3 [M+H]⁺.

### Step h): synthesis of tert-butyl (E)-(1-(5-(3-(benzyloxy)-4-methoxyphenyl)-4-(4-cyano-3-fluorophenyl)pyridin-2-yl)piperidin-4-yl)(4-(3-oxo-3-(((tetrahydro-2H-pyran-2-yl)oxy)amino)prop-1-en-1-yl)benzyl)carbamate

(E)-3-(4-(((1-(5-(3-(benzyloxy)-4-methoxyphenyl)-4-(4-cyano-3-fluorophenyl)pyridin-2-yl)piperidin-4-yl)(tert-butoxycarbonyl)amino)methyl)phenyl)acrylic acid (215 mg, 0.28 mmol), O-(tetrahydro-2H-pyran-2-yl)hydroxylamine (40 mg, 0.33 mmol), 2-(7-azabenzotriazolyl)-N,N,N′,N′-tetramethyluronium hexafluorophosphate (128 mg, 0.33 mmol), and ethyldiisopropylamine (109 mg, 0.84 mmol) were added to DMF (8 mL). The mixture was reacted at room temperature for 0.5 hours and monitored by LCMS until the starting materials were consumed. The reaction solution was poured into water, and ethyl acetate was added for extraction. The organic phase was washed with saturated brine, and the solvent was removed. The residue was mixed with silica gel and purified by column separation on silica gel to afford tert-butyl (E)-(1-(5-(3-(benzyloxy)-4-methoxyphenyl)-4-(4-cyano-3-fluorophenyl)pyridin-2-yl)piperidin-4-yl)(4-(3-oxo-3-(((tetrahydro-2H-pyran-2-yl)oxy)amino)prop-1-en-1-yl)benzyl)carbamate with a yield of 65.83%.

ESI-MS (m/z)=868.4 [M+H]⁺.

### Step i): synthesis of (E)-3-(4-(((1-(4-(4-cyano-3-fluorophenyl)-5-(3-hydroxy-4-methoxyphenyl)pyridin-2-yl)piperidin-4-yl)amino)methyl)phenyl)-N-hydroxyacrylamide formate

Tert-butyl (E)-(1-(5-(3-(benzyloxy)-4-methoxyphenyl)-4-(4-cyano-3-fluorophenyl)pyridin-2-yl)piperidin-4-yl)(4-(3-oxo-3-(((tetrahydro-2H-pyran-2-yl)oxy)amino)prop-1-en-1-yl)benzyl)carbamate (120 mg, 0.14 mmol) was added to DCM (8 mL), followed by boron tribromide (0.28 g, 1.12 mmol) at −78° C., and the reaction was maintained under a dry ice bath for 0.5 hours and monitored by LCMS until the starting materials were consumed. The reaction was quenched with methanol, then the solvent was removed, and the residue was purified by Prep-HPLC (separation method 3) to afford (E)-3-(4-(((1-(4-(4-cyano-3-fluorophenyl)-5-(3-hydroxy-4-methoxyphenyl)pyridin-2-yl)piperidin-4-yl)amino)methyl)phenyl)-N-hydroxyacrylamide formate with a yield of 66.18%.

¹H NMR (400 MHZ, Methanol-d₄) δ8.53 (s, 1H), 8.46 (s, 1H), 8.14 (s, 1H), 7.70-7.58 (m, 3H), 7.53 (t, J=11.1 Hz, 2H), 7.22 (dd, J=10.2, 1.5 Hz, 1H), 7.15 (dd, J=8.1, 1.5 Hz, 1H), 6.88-6.80 (m, 2H), 6.53 (d, J=8.0 Hz, 1H), 6.50 (dt, J=4.4, 2.4 Hz, 2H), 4.52 (d, J=13.6 Hz, 2H), 4.21 (s, 2H), 3.83 (s, 3H), 3.32 (s, 1H), 2.99 (t, J=12.7 Hz, 2H), 2.27-2.14 (m, 2H), 1.65 (q, J=12.1 Hz, 2H).

ESI-MS (m/z)=594.2 [M+H]⁺.

### Example 81

### Preparation of (E)-3-(3-((1-(3-cyano-4-(4-cyano-3-fluorophenyl)-5-(3-hydroxy-4-methoxyphenyl)pyridin-2-yl)piperidin-4-yl)amino)phenyl)-N-hydroxyacrylamide formate

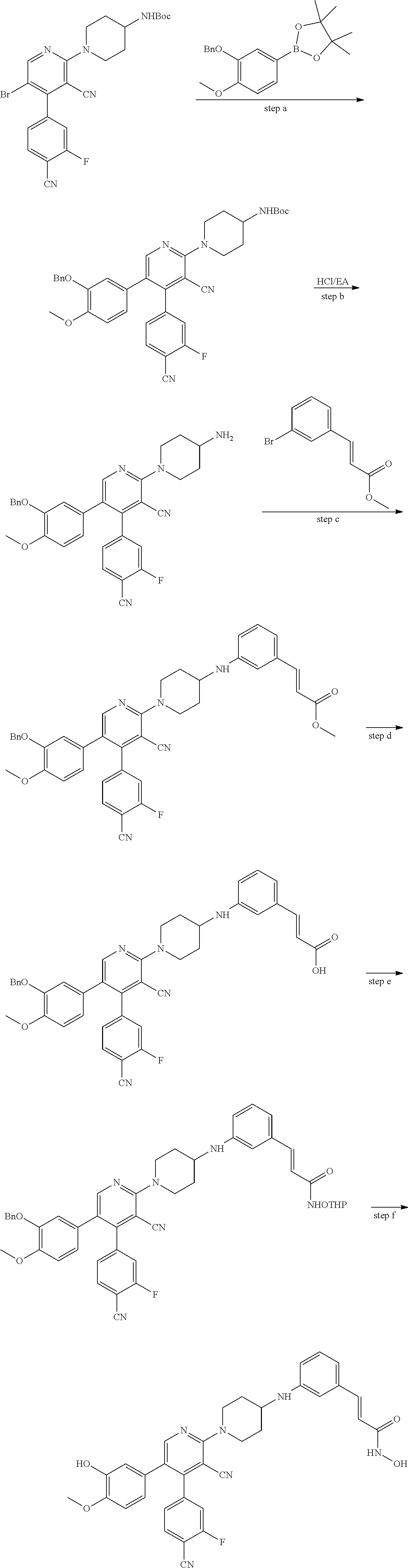

### Step a): preparation of tert-butyl (1-(5-(3-(benzyloxy)-4-methoxyphenyl)-3-cyano-4-(4-cyano-3-fluorophenyl)pyridin-2-yl)piperidin-4-yl)carbamate

The product of step c in Example 37 tert-butyl (1-(5-bromo-3-cyano-4-(4-cyano-3-fluorophenyl)pyridin-2-yl)piperidin-4-yl)carbamate (558.0 mg, 1.12 mmol) and 2-(3-(benzyloxy)-4-methoxyphenyl)-4,4,5,5-tetramethyl-1,3,2-dioxaborolane (280.1 mg, 1.12 mmol), Cs₂CO₃ (1.1 g, 3.36 mmol) and Pd(dppf)Cl₂ (82.0 mg, 0.11 mmol) were dissolved in 1,4-dioxane (8 mL) and water (1 mL), the reaction solution was purged with nitrogen three times and then heated to 100° C. After 2 hours, the reaction was completed as indicated by LC-MS, and the reaction solution was cooled to room temperature. Water (9 mL) was added, and the mixture was extracted with ethyl acetate (8 mL×3). The organic layers were combined, washed with saturated brine (8 mL), dried over anhydrous sodium sulfate, filtered, and concentrated under vacuum. The residue was purified by silica gel chromatography to afford tert-butyl (1-(5-(3-(benzyloxy)-4-methoxyphenyl)-3-cyano-4-(4-cyano-3-fluorophenyl)pyridin-2-yl)piperidin-4-yl)carbamate with a yield of 80.6%.

ESI-MS m/z=634.3 [M+H]⁺.

### Step b): preparation of 2-(4-aminopiperidin-1-yl)-5-(3-(benzyloxy)-4-methoxyphenyl)-4-(4-cyano-3-fluorophenyl)nicotinonitrile

Tert-butyl (1-(5-(3-(benzyloxy)-4-methoxyphenyl)-3-cyano-4-(4-cyano-3-fluorophenyl)pyridin-2-yl)piperidin-4-yl)carbamate (200 mg, 0.36 mmol) and 4M hydrochloric acid solution in ethyl acetate (5 mL) were added to a reaction flask, and the mixture was stirred at room temperature for 1 hour. The mixture was concentrated to afford 2-(4-aminopiperidin-1-yl)-5-(3-(benzyloxy)-4-methoxyphenyl)-4-(4-cyano-3-fluorophenyl)nicotinonitrile with a yield of 92%.

ESI-MS m/z=534.2 [M+H]⁺.

### Step c): preparation of methyl (E)-3-(3-((1-(5-(3-(benzyloxy)-4-methoxyphenyl)-3-cyano-4-(4-cyano-3-fluorophenyl)pyridin-2-yl))piperidin-4-yl)amino)phenyl)acrylate

2-(4-Aminopiperidin-1-yl)-5-(3-(benzyloxy)-4-methoxyphenyl)-4-(4-cyano-3-fluorophenyl)nicotinonitrile (178 mg, 0.32 mmol) was dissolved in toluene (3 mL), to the solution were added methyl (E)-3-(3-bromophenyl)acrylate (77.2 mg, 0.32 mmol), cesium carbonate (208.6 mg, 0.64 mmol), tris(dibenzylideneacetone)dipalladium (58.6 mg, 0.06 mmol), and 2-dicyclohexylphosphino-2′,6′-diisopropoxy-1,1′-biphenyl (30.5 mg, 0.06 mmol), and the mixture was refluxed overnight under nitrogen. After the reaction was completed as indicated by LCMS, the mixture was concentrated to dryness under reduced pressure. The residue was purified by silica gel chromatography to afford methyl (E)-3-(3-((1-(5-(3-(benzyloxy)-4-methoxyphenyl)-3-cyano-4-(4-cyano-3-fluorophenyl)pyridin-2-yl))piperidin-4-yl)amino)phenyl)acrylate with a yield of 73.3%.

ESI-MS m/z=694.3 [M+H]⁺.

### Step d): preparation of (E)-3-(3-((1-(5-(3-(benzyloxy)-4-methoxyphenyl)-3-cyano-4-(4-cyano-3-fluorophenyl)pyridin-2-yl)piperidin-4-yl)amino)phenyl)acrylic acid

Methyl (E)-3-(3-((1-(5-(3-(benzyloxy)-4-methoxyphenyl)-3-cyano-4-(4-cyano-3-fluorophenyl)pyridin-2-yl))piperidin-4-yl)amino)phenyl)acrylate (135 mg, 0.20 mmol) was dissolved in a mixture of THF/H₂O=5:1 (3 mL), then lithium hydroxide (48 mg, 2 mmol) was added, and the mixture was stirred overnight at room temperature. The pH of the solution was adjusted to 2-3 with 2 N HCl, and the mixture was then extracted with ethyl acetate. The organic phase was washed once with brine and subsequently concentrated under reduced pressure. The residue was used directly for the next step.

ESI-MS m/z=680.3 [M+H]⁺.

### Step e): preparation of (E)-3-(3-((1-(5-(3-(benzyloxy)-4-methoxyphenyl)-3-cyano-4-(4-cyano-3-fluorophenyl)pyridin-2-yl)piperidin-4-yl)amino)phenyl)-N-((tetrahydro-2H-pyran-2-yl)oxy) acrylamide

(E)-3-(3-((1-(5-(3-(benzyloxy)-4-methoxyphenyl)-3-cyano-4-(4-cyano-3-fluorophenyl)pyridin-2-yl)piperidin-4-yl)amino)phenyl)acrylic acid (80 mg, 0.12 mmol) was dissolved in DMF (2 mL), followed by adding HATU (54.75 mg, 0.14 mmol) and O-(tetrahydro-2H-pyran-2-yl)hydroxylamine (16.9 mg, 0.14 mmol), and the reaction was stirred at room temperature for 1 hour, then quenched with water. The mixture was extracted with ethyl acetate, and the organic phase was washed twice with water, concentrated under reduced pressure and the residue was purified by silica gel column to afford (E)-3-(3-((1-(5-(3-(benzyloxy)-4-methoxyphenyl)-3-cyano-4-(4-cyano-3-fluorophenyl)pyridin-2-yl)piperidin-4-yl)amino)phenyl)-N-((tetrahydro-2H-pyran-2-yl)oxy) acrylamide with a yield of 85%.

ESI-MS m/z=779.3 [M+H]⁺.

### Step f): preparation of (E)-3-(3-((1-(3-cyano-4-(4-cyano-3-fluorophenyl)-5-(3-hydroxy-4-methoxyphenyl)pyridin-2-yl)piperidin-4-yl)amino)phenyl)-N-hydroxyacrylamide formate

(E)-3-(3-((1-(5-(3-(Benzyloxy)-4-methoxyphenyl)-3-cyano-4-(4-cyano-3-fluorophenyl)pyridin-2-yl)piperidin-4-yl)amino)phenyl)-N-((tetrahydro-2H-pyran-2-yl)oxy) acrylamide (80 mg, 0.1 mmol) was dissolved in a solution of HCl/EA (3 mL, 2M), the mixture was reacted for 1 hour at room temperature. Solid was formed in the reaction solution, and the reaction was completed as indicated by LC-MS detection. The reaction solution was then filtered, and the solid was purified by Prep-HPLC (separation method 3) to afford (E)-3-(3-((1-(3-cyano-4-(4-cyano-3-fluorophenyl)-5-(3-hydroxy-4-methoxyphenyl)pyridin-2-yl)piperidin-4-yl)amino)phenyl)-N-hydroxyacrylamide formate with a yield of 55.2%.

¹H NMR (400 MHZ, DMSO-d₆) δ8.52 (s, 1H), 8.40 (s, 1H), 7.95-7.84 (m, 1H), 7.61 (dd, J=10.0, 1.5 Hz, 1H), 7.34 (d, J=15.8 Hz, 1H), 7.26 (dd, J=8.0, 1.6 Hz, 1H), 7.16 (t, J=7.8 Hz, 1H), 6.88-6.78 (m, 3H), 6.76-6.68 (m, 1H), 6.51-6.43 (m, 2H), 6.38 (d, J=15.8 Hz, 1H), 4.21 (d, J=12.8 Hz, 2H), 3.70 (s, 3H), 3.60 (s, 1H), 3.27 (t, J=12.2 Hz, 2H), 2.05 (d, J=11.8 Hz, 2H), 1.53 (q, J=11.4 Hz, 2H).

ESI-MS m/z=605.2 [M+H]⁺.

### Example 82

(E)-3-(4-((1-(3-Cyano-4-(4-cyano-3-fluorophenyl)-5-(3-hydroxy-4-methoxyphenyl)pyridin-2-yl)piperidin-4-yl)amino)phenyl)-N-hydroxyacrylamide formate was prepared according to the synthetic method of Example 81, and the structure and characterization data are as follows:

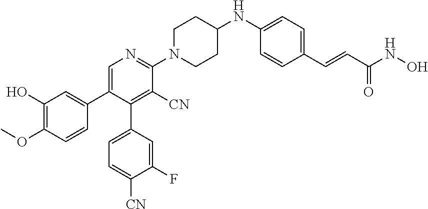 [SMILES: COc1ccc(-c2cnc(N3CCC(Nc4ccc(/C=C/C(=O)NO)cc4)CC3)c(C#N)c2-c2ccc(C#N)c(F)c2)cc1O]

¹H NMR (400 MHZ, DMSO-d₆) δ10.45 (s, 1H), 8.98 (s, 1H), 8.43 (s, 1H), 7.95 (t, J=7.4 Hz, 1H), 7.66 (dd, J=10.0, 1.4 Hz, 1H), 7.36-7.23 (m, 4H), 6.81 (d, J=8.0 Hz, 1H), 6.65 (d, J=8.2 Hz, 2H), 6.48 (d, J=8.4 Hz, 2H), 6.14 (d, J=12.0 Hz, 1H), 4.22 (d, J=13.2 Hz, 2H), 3.72 (s, 3H), 3.64 (s, 1H), 3.31 (t, J=11.8 Hz, 2H), 2.07 (d, J=11.8 Hz, 2H), 1.63-1.46 (m, 2H).

ESI-MS m/z=605.2 [M+H]⁺.

### Example 83

2-((1-(3-Cyano-4-(4-cyano-3-fluorophenyl)-5-(3-hydroxy-4-methoxyphenyl)pyridin-2-yl)piperidin-4-yl)amino)-N-hydroxypyrimidine-5-carboxamide formate was prepared according to the synthetic method of Example 81, and the structure and characterization data are as follows:

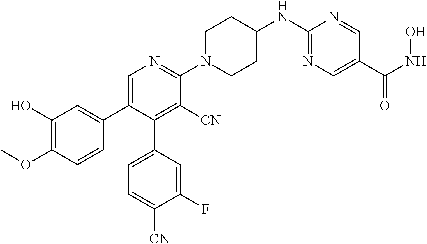 [SMILES: COc1ccc(-c2cnc(N3CCC(Nc4ncc(C(=O)NO)cn4)CC3)c(C#N)c2-c2ccc(C#N)c(F)c2)cc1O]

¹H NMR (400 MHZ, Methanol-d₄) δ ppm 8.64 (s, 2H), 8.52 (s, 1H), 8.41 (s, 1H), 7.74 (dd, J=8.0, 6.6 Hz, 1H), 7.37 (dd, J=9.6, 1.6 Hz, 1H), 7.24 (dd, J=8.0, 1.6 Hz, 1H), 6.87-6.76 (m, 1H), 6.56-6.46 (m, 2H), 4.35 (d, J=13.4 Hz, 2H), 4.17 (td, J=10.6, 5.4 Hz, 1H), 3.81 (s, 3H), 3.30-3.24 (m, 2H), 2.21-2.11 (m, 2H), 1.84-1.71 (m, 2H).

ESI-MS m/z=581.2 [M+H]⁺.

### Example 84

### Preparation of (E)-3-(4-(((1-(3′,4″-dicyano-3″-fluoro-3-hydroxy-4-methoxy-[1,1′:2′,1″-terphenyl]-4′-yl)piperidin-4-yl)amino)methyl)phenyl)-N-hydroxyacrylamide

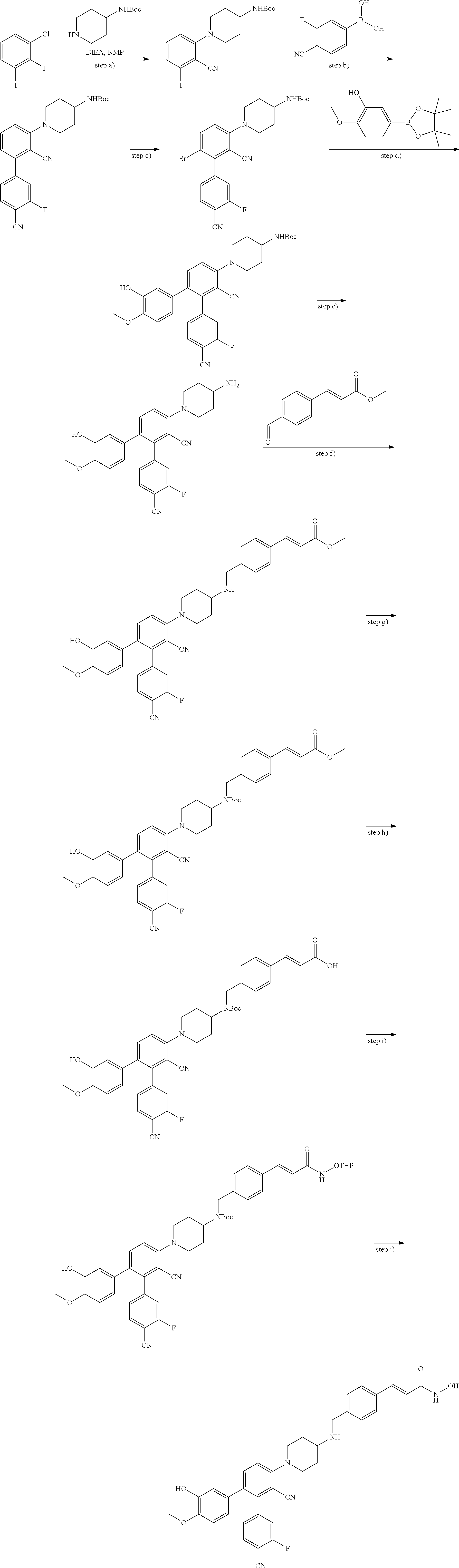

### Step a): preparation of tert-butyl (1-(2-cyano-3-iodophenyl)piperidin-4-yl)carbamate

2-Chloro-6-iodobenzonitrile (2 g, 7.63 mmol), tert-butyl piperidin-4-ylcarbamate (2.29 g, 11.45 mmol), and DIEA (3.94 g, 30.52 mmol) were added to a reaction flask containing NMP (20 mL), and the solution was stirred at 100° C. for 24 hours. The reaction solution was quenched by adding water (20 mL), extracted with ethyl acetate (20 mL×2). And the organic phases were combined, washed with saturated brine (15 mL×2), dried over anhydrous sodium sulfate, filtered, and the residue was purified by silica gel chromatography (eluent: petroleum ether/ethyl acetate=1/1) to afford tert-butyl (1-(2-cyano-3-iodophenyl)piperidin-4-yl)carbamate with a yield of 37%. ESI-MS m/z=428.1 [M+H]⁺.

### Step b): preparation of tert-butyl (1-(2,4′-dicyano-3′-fluoro-[1,1′-biphenyl]-3-yl)piperidin-4-yl)carbamate

Tert-butyl (1-(2-cyano-3-iodophenyl)piperidin-4-yl)carbamate (1.3 g, 3.04 mmol), 4-cyano-3-fluorophenylboronic acid (0.60 g, 3.65 mmol), Pd₂(dppf)Cl₂ (0.22 g, 0.30 mmol), and cesium carbonate (1.98 g, 6.08 mmol) were added to a microwave reaction tube containing dioxane (2 mL) and water (0.4 mL). The mixture was stirred at 80° C. for 45 minutes. The reactionsolution was concentrated after adding silica gel, and the residue was purified by silica gel chromatography (eluent: petroleum ether/ethyl acetate=1/1) to afford tert-butyl (1-(2,4′-dicyano-3′-fluoro-[1,1′-biphenyl]-3-yl)piperidin-4-yl)carbamate with a yield of 66%.

ESI-MS m/z=421.2 [M+H]⁺.

### Step c): preparation of tert-butyl (1-(6-bromo-2,4′-dicyano-3′-fluoro-[1,1′-biphenyl]-3-yl)piperidin-4-yl)carbamate

Tert-butyl (1-(2,4′-dicyano-3′-fluoro-[1,1′-biphenyl]-3-yl)piperidin-4-yl)carbamate (840 mg, 2.00 mmol) and NBS (0.42 g, 2.36 mmol) were added to a reaction flask containing DMF (10 mL), and the mixture was stirred at room temperature for 2 hours. The reaction was quenched by adding water (20 mL), extracted with ethyl acetate (20 mL×2). And the organic phases were combined, washed with saturated brine (15 mL×2), dried over anhydrous sodium sulfate, filtered, and the residue was purified by silica gel chromatography (eluent: petroleum ether/ethyl acetate=1/1) to afford tert-butyl (1-(6-bromo-2,4′-dicyano-3′-fluoro-[1,1′-biphenyl]-3-yl)piperidin-4-yl)carbamate with a yield of 67%.

ESI-MS m/z=499.1 [M+H]⁺.

### Step d): preparation of tert-butyl (1-(3,4″-dicyano-3-fluoro-3-hydroxy-4-methoxy-[1,1:2,1-terphenyl]-4-yl)piperidin-4-yl)carbamate

Tert-butyl (1-(6-bromo-2,4′-dicyano-3′-fluoro-[1,1′-biphenyl]-3-yl)piperidin-4-yl)carbamate (620 mg, 1.24 mmol), 2-methoxy-5-(4,4,5,5-tetramethyl-1,3,2-dioxaborolan-2-yl)phenol (0.31 g, 1.24 mmol), Pd₂(dppf)Cl₂ (0.091 g, 0.12 mmol), and cesium carbonate (0.81 g, 2.48 mmol) were added to a microwave reaction tube containing 1,4-dioxane (8 mL) and water (1.6 mL), and the mixture was subjected to microwave heating at 110° C. and stirred for 45 minutes. The reaction solution was concentrated after addition of silica gel, and the residue was purified by silica gel chromatography (eluent: dichloromethane/methanol=10/1) to afford tert-butyl (1-(3,4″-dicyano-3-fluoro-3-hydroxy-4-methoxy-[1,1:2,1-terphenyl]-4-yl)piperidin-4-yl)carbamate with a yield of 98%.

ESI-MS m/z=443.2 [M+H]⁺.

### Step e): preparation of 6-(4-aminopiperidin-1-yl)-2-(4-cyano-3-fluorophenyl)-3-3-hydroxy-4-methoxyphenyl)benzonitrile

Tert-butyl (1-(3,4″-dicyano-3-fluoro-3-hydroxy-4-methoxy-[1,1:2,1-terphenyl]-4-yl)piperidin-4-yl)carbamate (620 mg, 1.18 mmol) was added to a reaction flask containing a 4M hydrochloric acid solution in ethyl acetate (10 mL), and the mixture was stirred at room temperature for 1 hour. Saturated sodium bicarbonate aqueous solution was added to adjust the pH to 7, followed by extraction with a mixture of DCM:MeOH=10:1 (20 mL×2). And the organic phases were combined and washed with saturated brine (15 mL×2), dried with anhydrous sodium sulfate, filtered, and concentrated to afford 6-(4-aminopiperidin-1-yl)-2-(4-cyano-3-fluorophenyl)-3-(3-hydroxy-4-methoxyphenyl)benzonitrile with a yield of 16%.

ESI-MS m/z=443.2 [M+H]⁺.

### Step f): preparation of methyl (E)-3-(4-(((1-(3′,4″-dicyano-3″-fluoro-3-hydroxy-4-methoxy-[1,1′:2′,1′-terphenyl]-4′-yl)piperidin-4-yl)amino)methyl)phenyl)acrylate

6-(4-Aminopiperidin-1-yl)-2-(4-cyano-3-fluorophenyl)-3-(3-hydroxy-4-methoxyphenyl)benzonitrile (240 mg, 0.54 mmol) and methyl (E)-3-(4-formylphenyl)acrylate (0.30 g, 1.60 mmol) were added to a reaction flask containing DCE (5 mL), and the mixture was stirred at room temperature for 1 hour, then the reaction solution was cooled to 0° C., and sodium cyanoborohydride (0.17 g, 2.71 mmol) was added, and the mixture was stirred at room temperature for 16 hours. The reaction solution was quenched by adding saturated sodium bicarbonate aqueous solution (5 mL), followed by extraction with ethyl acetate (20 mL×2). And the organic phases were combined and washed with saturated brine (15 mL×2), dried over anhydrous sodium sulfate, filtered, and concentrated. The residue was purified by silica gel chromatography (eluent: petroleum ether/ethyl acetate=1/1) to afford methyl (E)-3-(4-(((1-(3′,4″-dicyano-3″-fluoro-3-hydroxy-4-methoxy-[1,1′:2′,1′-terphenyl]-4′-yl)piperidin-4-yl)amino)methyl)phenyl)acrylate with a yield of 63%.

ESI-MS m/z=617.2 [M+H]⁺.

### Step g): preparation of methyl (E)-3-(4-tert-butoxycarbonyl (1-(3′,4″-dicyano-3″-fluoro-3-hydroxy-4-methoxy-1,1′:2′,1″-terphenyl]-4′-yl)piperidin-4-yl)amino)methyl)phenyl)acrylates

Methyl (E)-3-(4-(((1-(3′,4″-dicyano-3″-fluoro-3-hydroxy-4-methoxy-[1,1′:2′,1″-terphenyl]-4′-yl)piperidin-4-yl)amino)methyl)phenyl)acrylate (150 mg, 0.24 mmol), di-tert-butyl dicarbonate (0.27 g, 1.22 mmol), and TEA (0.097 g, 0.96 mmol) were added to a reaction flask containing dichloromethane (4 mL), and the mixture was stirred at room temperature for 1 hour. The reactionsolution was concentrated to afford methyl (E)-3-(4-tert-butoxycarbonyl (1-(3′,4″-dicyano-3″-fluoro-3-hydroxy-4-methoxy-1,1′:2′,1″-terphenyl]-4′-yl)piperidin-4-yl)amino)methyl)phenyl)acrylates with a yield of 93%.

ESI-MS m/z=717.3 [M+H]⁺.

### Step h): preparation of (E)-3-(4-(tert-butoxycarbonyl)(1-(3′,4″-dicyano-3″-fluoro-3-hydroxy-4-methoxy-[1,1:2′,1″-terphenyl]-4′-yl)piperidin-4-yl)amino)methyl)phenyl)acrylic acid

methyl (E)-3-(4-tert-butoxycarbonyl (1-(3′,4″-dicyano-3″-fluoro-3-hydroxy-4-methoxy-1,1′:2′,1″-terphenyl]-4′-yl)piperidin-4-yl)amino)methyl)phenyl)acrylates (160 mg, 0.22 mmol) and lithium hydroxide (0.11 g, 4.42 mmol) were added to a reaction flask containing a mixture of THF (3 mL), MeOH (1.8 mL), and water (1.2 mL), and the mixture was stirred at room temperature for 1 hour. To the reaction solution, was added 2M HCl solution dropwise to adjust the pH to 4, followed by extraction with dichloromethane (20 mL×2). And the organic phases were combined and washed with saturated brine (15 mL×2), dried over anhydrous sodium sulfate, filtered, and concentrated to afford (E)-3-(4-(tert-butoxycarbonyl)(1-(3′,4″-dicyano-3″-fluoro-3-hydroxy-4-methoxy-[1,1:2′,1″-terphenyl]-4′-yl)piperidin-4-yl)amino)methyl)phenyl)acrylic acid with a yield of 91%.

ESI-MS m/z=703.2 [M+H]⁺.

### Step i): preparation of tert-butyl (E)-(1-(3′,4″-dicyano-3″-fluoro-3-hydroxy-4-methoxy-[1,1′:2′,1″-terphenyl]-4′-yl)piperidin-4-yl)(4-(3-oxo-3-(((tetrahydro-2H-pyran-2-yl)oxy)amino)prop-1-en-1-yl)benzyl)carbamate

(E)-3-(4-(tert-butoxycarbonyl)(1-(3′,4″-dicyano-3″-fluoro-3-hydroxy-4-methoxy-[1,1:2′,1″-terphenyl]-4′-yl)piperidin-4-yl)amino)methyl)phenyl)acrylic acid (140 mg, 0.20 mmol), HATU (0.11 g, 0.30 mmol), DIEA (0.10 g, 0.80 mmol), and O-(tetrahydro-2H-pyran-2-yl)hydroxylamine (0.047 g, 0.40 mmol) were added to a reaction flask containing DMF (5 mL), and the mixture was stirred at room temperature for 1 hour. Water (20 mL) was added to quench the reaction, followed by extraction with ethyl acetate (20 mL×2). The organic phases were combined and washed with saturated brine (15 mL×2), dried over anhydrous sodium sulfate, filtered, and the residue was purified by silica gel chromatography (eluent: DCM/MeOH=10/1) to afford tert-butyl (E)-(1-(3′,4″-dicyano-3″-fluoro-3-hydroxy-4-methoxy-[1,1′;2′,1″-terphenyl]-4′-yl)piperidin-4-yl)(4-(3-oxo-3-(((tetrahydro-2H-pyran-2-yl)oxy)amino)prop-1-en-1-yl)benzyl)carbamate with a yield of 93%.

ESI-MS m/z=802.3 [M+H]⁺.

### Step j): preparation of ((E)-3-(4-(((1-(3′,4″-dicyano-3″-fluoro-3-hydroxy-4-methoxy-[1,1′:2′,1″-terphenyl]-4′-yl)piperidin-4-yl)amino)methyl)phenyl)-N-hydroxyacrylamide

Tert-butyl (E)-(1-(3′,4″-dicyano-3″-fluoro-3-hydroxy-4-methoxy-[1,1′:2′,1″-terphenyl]-4′-yl)piperidin-4-yl)(4-(3-oxo-3-(((tetrahydro-2H-pyran-2-yl)oxy)amino)prop-1-en-1-yl)benzyl)carbamate (140 mg, 0.17 mmol) and 4M hydrochloric acid solution in ethyl acetate (12.00 g, 329.13 mmol) were added to a reaction flask, and the mixture was stirred at room temperature for 1 hour. After concentrating, the residue was purified by Prep-HPLC (separation method Method 3) to afford (E)-3-(4-(((1-(3′,4″-dicyano-3″-fluoro-3-hydroxy-4-methoxy-[1,1′:2′,1″-terphenyl]-4′-yl)piperidin-4-yl)amino)methyl)phenyl)-N-hydroxyacrylamide formate with a yield of 45%.

¹H NMR (400 MHZ, DMSO-d₆) δ ppm 10.72 (s, 1H), 8.97 (s, 1H), 8.51 (s, 1H), 8.19 (s, 1H), 7.89 (t, J=7.4 Hz, 1H), 7.63-7.49 (m, 4H), 7.49-7.34 (m, 3H), 7.27 (d, J=8.6 Hz, 1H), 7.21 (dd, J=8.0, 1.4 Hz, 1H), 6.77 (d, J=8.3 Hz, 1H), 6.49-6.34 (m, 3H), 3.85 (s, 2H), 3.70 (s, 3H), 3.55 (s, 2H), 2.90 (t, J=11.3 Hz, 2H), 2.73-2.63 (m, 1H), 2.06-1.95 (m, 2H), 1.54 (dq, J=106.2, 9.9 Hz, 2H).

ESI-MS m/z=618.2 [M+H]⁺.

### Example 85

### Preparation of (E)-3-(4-(4-amino-1-(3-cyano-4-(4-cyano-3-fluorophenyl)-5-(3-hydroxy-4-methoxyphenyl)pyridin-2-yl))piperidin-4-yl)phenyl)-N-hydroxyacrylamide formate

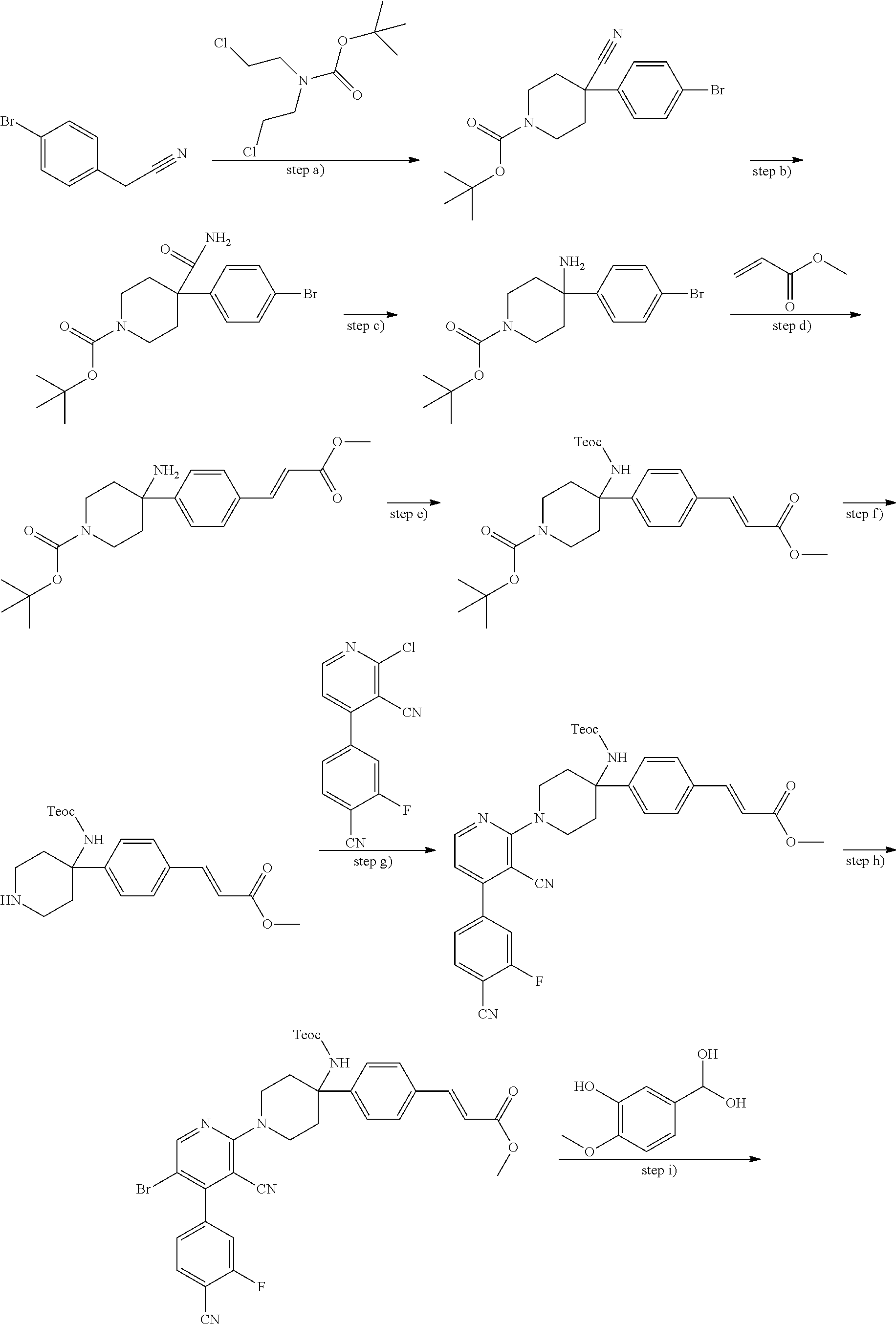

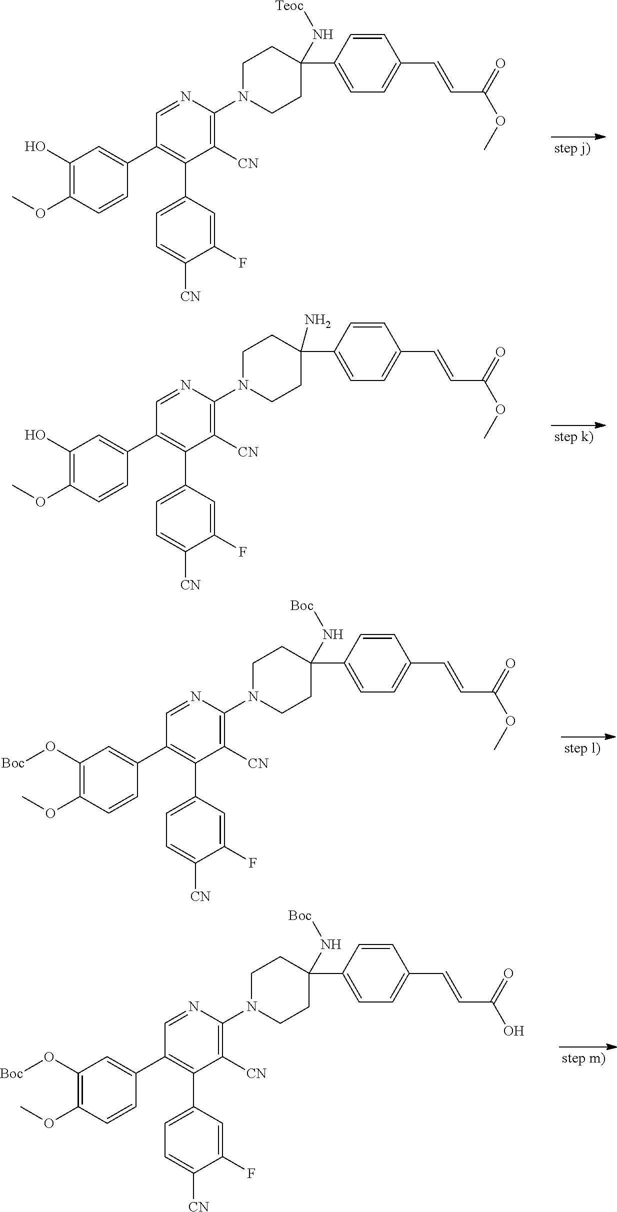

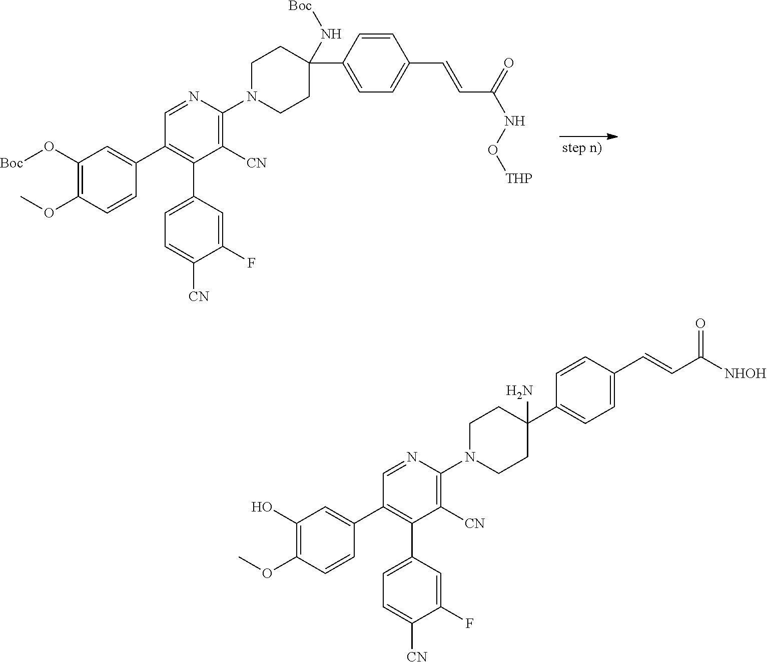

### Step a) synthesis of tert-butyl 4-(4-bromophenyl)-4-cyanopiperidine-1-carboxylate

2-(4-Bromophenyl) acetonitrile (5 g, 25.50 mmol) and tert-butyl N,N-bis(2-chloroethyl)carbamate (7.10 g, 29.32 mmol) were added to DMF (75 mL), then sodium hydride (1.84 g, 76.5 mmol) was added portion-wise at 0° C. After the addition, the temperature was raised to 65° C. and the reaction was stirred for 1.5 hours. Then cooling to room temperature the reaction was quenched with water, extracted with EA, and the organic phases were combined, concentrated and purified by column chromatography on silica gel to afford tert-butyl 4-(4-bromophenyl)-4-cyanopiperidine-1-carboxylate with a yield of 83.74%.

ESI-MS (m/z)=365.1 [M+H]⁺

### Step b) synthesis of tert-butyl 4-(4-bromophenyl)-4-carbamoylpiperidine-1-carboxylate

Tert-butyl 4-(4-bromophenyl)-4-cyanopiperidine-1-carboxylate (5 g, 13.7 mmol), potassium hydroxide (1537.4 mg, 27.4 mmol) were added to DMSO (5 mL), then hydrogen peroxide (465.9 mg, 13.7 mmol) was added slowly. And the reaction was stirred at room temperature for 1 h, and the disappearance of the starting material was monitored by TLC (petroleum ether:ethyl acetate=3:1). The reaction was quenched with water, the solution was extracted by EA. The organic phases were combined, and after the solvent was pumped off by an oil pump, the crude product was used directly for the next step.

ESI-MS (m/z)=383.1 [M+H]⁺.

### Step c) synthesis of tert-butyl 4-amino-4-(4-bromophenyl)piperidine-1-carboxylate

Potassium hydroxide (1641.2 mg, 29.3 mmol) was dissolved in a mixture of ACN: H₂O=1:4 (40 mL), and the crude product of Step b, tert-butyl 4-(4-bromophenyl)-4-carbamoylpiperidine-1-carboxylate (2.5 g, 6.5 mmol) was added and stirred for 10 minutes, then 1,3-dibromo-5,5-dimethylhydantoin (1022.2 mg, 3.6 mmol) was added portion-wise, and the mixture was reacted at room temperature for 1 hour and monitored by TLC (ethyl acetate as the developing agent). After the disappearance of the starting material, a more polar spot (Rf: 0.15) (ninhydrin colour-development) appeared. The solution was added with sodium sulfite (0.082 g, 0.65 mmol) and stirred for 15 minutes. After the addition of potassium phosphate (1.7 g, 7.9 mmol), the mixture was extracted with ethyl acetate. And the organic phases were combined, washed with a brine, and concentrated to remove the solvent. The residue was mixed with silica gel and purified by column chromatography on silica gel to afford tert-butyl 4-amino-4-(4-bromophenyl)piperidine-1-carboxylate with a yield of 77.95%.

ESI-MS (m/z)=355.1 [M+H]⁺.

### Step d) synthesis of tert-butyl (E)-4-amino-4-(4-(3-methoxy-3-oxoprop-1-en-1-yl)phenyl)piperidine-1-carboxylate

Tert-butyl 4-amino-4-(4-bromophenyl)piperidine-1-carboxylate (1400 mg, 3.9 mmol), methyl acrylate (430.5 mg, 5.0 mmol), tetrakis(triphenylphosphine) palladium (115.6 mg, 0.10 mmol), and triethylamine (202.4 mg, 2.0 mmol) were added to a mixture of tetrahydrofuran:water=5:1 (5 mL). The reactionsolution was heated to 110° C. and reacted for 4 hours and monitored by LCMS until the starting materials were consumed The reaction solution was quenched with water, extracted with ethyl acetate. And the organic phases were combined, concentrated, and the residue was mixed with silica gel and purified by column chromatography on silica gel to afford tert-butyl (E)-4-amino-4-(4-(3-methoxy-3-oxoprop-1-en-1-yl)phenyl)piperidine-1-carboxylate with a yield of 82.13%.

ESI-MS (m/z)=361.1 [M+H]⁺.

### Step e) synthesis of tert-butyl (E)-4-(4-(3-methoxy-3-oxoprop-1-en-1-yl)phenyl)-4-(((trimethylsilyl) ethoxy)carbonyl)amino)piperidine-1-carboxylate

Tert-butyl (E)-4-amino-4-(4-(3-methoxy-3-oxoprop-1-en-1-yl)phenyl)piperidine-1-carboxylate (500 mg, 0.80 mmol), 2,5-dioxopyrrolidin-1-yl 2-(trimethylsilyl)ethyl carbonate (1415.9 mg, 5.5 mmol) and sodium bicarbonate (982.9 mg, 11.7 mmol) were added to DMF (40 mL). The mixture was reacted at room temperature overnight, and the reaction was monitored by TLC, which showed the disappearance of the starting material and the appearance of a new spot. The mixture was extracted with ethyl acetate. The organic phases were combined, and concentrated. The residue was mixed with silica gel and purified by column chromatography on silica gel to afford tert-butyl (E)-4-(4-(3-methoxy-3-oxoprop-1-en-1-yl)phenyl)-4-(((trimethylsilyl) ethoxy)carbonyl)amino)piperidine-1-carboxylate with a yield of 73.22%.

ESI-MS (m/z)=491.2 [M+H]⁺.

Step f) synthesis of methyl (E)-3-(4-(4-(((trimethylsilyl) ethoxy)carbonyl)amino)piperidin-4-yl)phenyl)acrylate Tert-butyl (E)-4-(4-(3-methoxy-3-oxoprop-1-en-1-yl)phenyl)-4-((trimethylsilyl) ethoxy)carbonyl)amino)piperidine-1-carboxylate (180 mg, 0.37 mmol) was added to 4N hydrochloric acid solution in ethyl acetate (5 mL), and the mixture was reacted at room temperature for 0.5 hours. Concentraed, and sodium bicarbonate aqueous solution was then added to adjust the pH to weakly basic. The mixture was extracted with EA. And the organic phases were combined, concentrated to remove the solvent to afford the crude product methyl (E)-3-(4-(4-((trimethylsilyl) ethoxy)carbonylamino)piperidin-4-yl)phenyl)acrylate with a yield of 83.04%.

ESI-MS (m/z)=391.2 [M+H]⁺.

### Step g) synthesis of methyl (E)-3-(4-(1-(3-cyano-4-(4-cyano-3-fluorophenyl)pyridin-2-yl)-4-((2-(trimethylsilyl) ethoxy)carbonyl)amino)piperidin-4-yl)phenyl)acrylate

Methyl (E)-3-(4-(4-(((2-(trimethylsilyl) ethoxy)carbonyl)amino)piperidin-4-yl)phenyl)prop-2-enonate (100 mg, 0.24 mmol), 2-chloro-4-(4-cyano-3-fluorophenyl)nicotinonitrile (86.86 mg, 0.34 mmol), and ethyl bis(2-(prop-2-yl)amine (62.04 mg, 0.48 mmol) were added to N-methyl-2-pyrrolidone (4 mL), and the reaction solution was heated to 110° C. and reacted overnight. The product was purified by column chromatography on silica gel to afford the product methyl (E)-3-(4-(1-(3-cyano-4-(4-cyano-3-fluorophenyl)pyridin-2-yl)-4-((2-(trimethylsilyl) ethoxy)carbonyl)amino)piperidin-4-yl)phenyl)acrylate.

ESI-MS (m/z)=612.2 [M+H]⁺.

### Step h) synthesis of methyl (E)-3-(4-(1-(5-bromo-3-cyano-4-(4-cyano-3-fluorophenyl)pyridin-2-yl)-4-(((2-(trimethylsilyl) ethoxy)carbonyl)amino)piperidin-4-yl)phenyl)acrylate

Methyl (E)-3-(4-(1-(3-cyano-4-(4-cyano-3-fluorophenyl)pyridin-2-yl)-4-{[(2-(trimethylsilyl) ethoxy)carbonyl]amino}piperidin-4-yl)phenyl)acrylate (100 mg, 0.160 mmol) and N-bromosuccinimide (34 mg, 0.2 mmol) were added to DMF (2 mL), the reaction was stirred at room temperature for 2 hours, and quenched with water, followed by extraction with ethyl acetate. The organic phases were combined, and concentrating was conducted to remove the solvent. The residue was mixed with silica gel and purified by column chromatography on silica gel to afford the product methyl (E)-3-(4-(1-(5-bromo-3-cyano-4-(4-cyano-3-fluorophenyl)pyridin-2-yl)-4-(((2-(trimethylsilyl) ethoxy)carbonyl)amino)piperidin-4-yl)phenyl)acrylate with a yield of 49.67%.

ESI-MS (m/z)=690.2 [M+H]⁺.

### Step i) synthesis of methyl (E)-3-(4-(1-(3-cyano-4-(4-cyano-3-fluorophenyl)-5-(3-hydroxy-4-methoxyphenyl)pyridin-2-yl)-4-{(2-(trimethylsilyl) ethyoxy)carbonyl)amino)piperidin-4-yl)phenyl)acrylate

Methyl (E)-3-(4-(1-(5-bromo-3-cyano-4-(4-cyano-3-fluorophenyl)pyridin-2-yl)-4-(((2-(trimethylsilyl) ethoxy)carbonyl)amino)piperidin-4-yl)phenyl)acrylate (49.25 mg, 0.071 mmol), 2-methoxy-5-(tetramethyl-1,3,2-dioxaborolan-2-yl)phenol (23.08 mg, 0.092 mmol), [1,1′-bis(diphenylphosphino)ferrocene]palladium dichloride (10.39 mg, 0.014 mmol), and cesium carbonate (41.64 mg, 0.13 mmol) were added to a mixture of dioxane:water=5:1 (3 mL), and the mixture was heated to to 105° C. with microwave and reacted for 1 hour. The reaction was monitored by LCMS until the starting materials were consumed, and the solvent was removed. The residue was mixed with silica gel and purified by a column chromatography on silica gel to afford the product methyl (E)-3-(4-(1-(3-cyano-4-(4-cyano-3-fluorophenyl)-5-(3-hydroxy-4-methoxyphenyl)pyridin-2-yl)-4-{(2-(trimethylsilyl)ethoxy)carbonyl]amino}piperidin-4-yl)phenyl)acrylate with a yield of 72.63%.

ESI-MS (m/z)=734.2 [M+H]⁺.

### Step j) synthesis of methyl (E)-3-(4-(4-amino-1-(3-cyano-4-(4-cyano-3-fluorophenyl)-5-(3-hydroxy-4-methoxyphenyl)pyridin-2-yl)piperidin-4-yl)phenyl)acrylate

Methyl (E)-3-(4-(1-(3-cyano-4-(4-cyano-3-fluorophenyl)-5-(3-hydroxy-4-methoxyphenyl)pyridin-2-yl)-4-{(2-(trimethylsilyl) ethoxy)carbonyl)amino)piperidin-4-yl)phenyl)acrylate (30 mg, 0.041 mmol), tetrabutylammonium fluoride (107.20 mg, 0.41 mmol) were added to DCM (5 mL), reacted for 1 h. Then water was added, and the mixture was extracted with ethyl acetate. The organic phases were combined, and the solvent was removed. The residue was first purified by a reverse-phase column chromatography, followed by purification by normal phase column chromatography on silica gel to afford methyl (E)-3-(4-(4-amino-1-(3-cyano-4-(4-cyano-3-fluorophenyl)-5-(3-hydroxy-4-methoxyphenyl)pyridin-2-yl)piperidin-4-yl)phenyl)acrylate with a yield of 72.63%. ESI-MS (m/z)=604.2 [M+H]⁺.

### Step k) synthesis of methyl (E)-3-(4-(4-{[(tert-butoxy)carbonyl)amino)-1-(5-(3-{[(tert-butoxy)carbonyl]oxy}-4-methoxyphenyl)-3-cyano-4-(4-cyano-3-fluorophenyl)pyridin-2-yl)piperidin-4-yl)phenyl)acrylate

Methyl (E)-3-(4-(4-amino-1-(3-cyano-4-(4-cyano-3-fluorophenyl)-5-(3-hydroxy-4-methoxyphenyl)pyridin-2-yl)piperidin-4-yl)phenyl)acrylate (20 mg, 0.33 mmol), di-tert-butyl dicarbonate (360 mg, 1.7 mmol) and sodium bicarbonate (28.53 mg, 0.33 mmol) were added to a mixture of THF: H₂O=4:1 (10 mL). The reaction solution was stirred at room temperature overnight, and the solvent was removed t under reduced pressure to afford the crude product methyl (E)-3-(4-(4-{[(tert-butoxy)carbonyl]amino}-1-(5-(3-{[(tert-butoxy)carbonyl]oxy}-4-methoxyphenyl)-3-cyano-4-(4-cyano-3-fluorophenyl)pyridin-2-yl)piperidin-4-yl)phenyl)acrylate with a yield of 79.06%.

ESI-MS (m/z)=804.3 [M+H]⁺.

### Step 1) synthesis of (E)-3-(4-(4-{[(tert-butoxy)carbonyl]amino}-1-(5-(3-{[(tert-butoxy)carbonyl)oxy)-4-methoxyphenyl)-3-cyano-4-(4-cyano-3-fluorophenyl)pyridin-2-yl)piperidin-4-yl)phenyl)acrylic acid

Methyl (E)-3-(4-(4-{[(tert-butoxy)carbonyl]amino}-1-(5-(3-{[(tert-butoxy)carbonyl)oxy}-4-methoxyphenyl)-3-cyano-4-(4-cyano-3-fluorophenyl)pyridin-2-yl)piperidin-4-yl)phenyl)acrylate (19 mg, 0.02 mmol), lithium hydroxide (3 mg, 0.1 mmol) were added to a mixture of THF: H₂O=4:1 (10 mL), and the reaction solution was stirred at room temperature overnight. The pH of the mixture was adjusted to 4-5 with dilute hydrochloric acid, and the mixture was extracted with ethyl acetate. The organic phases were combined, and the solvent was removed to afford the crude product, (E)-3-(4-(4-{[(tert-butoxy)carbonyl]amino}-1-(5-(3-{[(tert-butoxycarbonyl)oxy)-4-methoxyphenyl)-3-cyano-4-(4-cyano-3-fluorophenyl)pyridin-2-yl)piperidin-4-yl)phenyl)acrylic acid with a yield of 80.25%.

ESI-MS (m/z)=790.3 [M+H]⁺.

### Step m) synthesis of tert-butyl (E)-(1-(5-(3-((tert-butoxycarbonyl)oxy)-4-methoxyphenyl)-3-cyano-4-(4-cyano-3-fluorophenyl)pyridin-2-yl))-4-(4-(3-oxo-3-(((tetrahydro-2H-pyran-2-yl)oxy)amino)prop-1-en-1-yl)phenyl)piperidin-4-yl)carbamate

(E)-3-(4-(4-{[(Tert-butoxy)carbonyl]amino}-1-(5-(3-{[(tert-butoxy)carbonyl]oxy}-4-methoxyphenyl)-3-cyano-4-(4-cyano-3-fluorophenyl)pyridin-2-yl)piperidin-4-yl)phenyl)acrylic acid (15 mg, 0.02 mmol), O-(tetrahydro-2H-pyran-2-yl)hydroxylamine (3 mg, 0.02 mmol), 2-(7-azabenzotriazolyl)-N,N,N′,N′-tetramethyluronium hexafluorophosphate (HATU, 10.2 mg, 0.03 mmol), and diisopropylethylamine (90 mg, 0.7 mmol) were added to DMF (8 mL), and the mixture was stirred at room temperature for 0.5 hours and monitored by LCMS until the starting materials were consumed. The reaction solution was poured into water, and extracted with EA. The organic phase was washed with saturated brine, and the solvent was removed. The residue was mixed with silica gel and purified by column chromatography on silica gel to afford the product tert-butyl (E)-(1-(5-(3-((tert-butoxycarbonyl)oxy)-4-methoxyphenyl)-3-cyano-4-(4-cyano-3-fluorophenyl)pyridin-2-yl))-4-(4-(3-oxo-3-(((tetrahydro-2H-pyran-2-yl)oxy)amino)prop-1-en-1-yl)phenyl)piperidin-4-yl)carbamate with a yield of 81.49%. ESI-MS (m/z)=889.5 [M+H]⁺.

### Step n) synthesis of (E)-3-(4-(4-amino-1-(3-cyano-4-(4-cyano-3-fluorophenyl)-5-(3-hydroxy-4-methoxyphenyl)pyridin-2-yl))piperidin-4-yl)phenyl)-N-hydroxyacrylamide formate

Tert-butyl (E)-(1-(5-(3-((tert-butoxycarbonyl)oxy)-4-methoxyphenyl)-3-cyano-4-(4-cyano-3-fluorophenyl)pyridin-2-yl))-4-(4-(3-oxo-3-(((tetrahydro-2H-pyran-2-yl)oxy)amino)prop-1-en-1-yl)phenyl)piperidin-4-yl)carbamate (15 mg, 0.01 mmol) was added to a solution of 4N hydrochloric acid solution in ethyl acetate (4 mL), and the mixture was reacted at room temperature for 0.5 hours, then the solvent was removed. The residue was purified with preparative HPLC to afford (E)-3-(4-(4-amino-1-(3-cyano-4-(4-cyano-3-fluorophenyl)-5-(3-hydroxy-4-methoxyphenyl)pyridin-2-yl))piperidin-4-yl)phenyl)-N-hydroxyacrylamide with a yield of 65.61%.

¹H NMR (400 MHZ, DMSO-d₆) δ 10.73 (s, 1H), 9.00 (s, 1H), 8.52 (s, 1H), 8.43 (s, 1H), 8.18 (s, 1H), 7.96 (t, J=7.4 Hz, 1H), 7.67 (dd, J=10.1, 1.4 Hz, 1H), 7.61 (d, J=8.3 Hz, 2H), 7.58-7.51 (m, 2H), 7.45 (d, J=15.8 Hz, 1H), 7.28 (dd, J=8.0, 1.4 Hz, 1H), 6.81 (d, J=8.1 Hz, 1H), 6.52-6.39 (m, 3H), 4.08-3.95 (m, 2H), 3.75 (d, J=12.0 Hz, 2H), 3.71 (s, 3H), 3.44 (q, J=6.9 Hz, 2H), 2.22-2.05 (m, 2H), 1.82 (d, J=13.1 Hz, 2H).

ESI-MS (m/z)=605.2 [M+H]⁺.

Example 86 was prepared according to the synthetic method of Example 85 (separation method 3), and the structure and characterization data are as follows:

### Preparation of (E)-3-(4-(2-(4-amino-1-(3-cyano-4-(4-cyano-3-fluorophenyl)-5-(3-hydroxy-4-methoxyphenyl)pyridin-2-yl)piperidin-4-yl)ethyl)phenyl)-N-hydroxyacrylamide formate

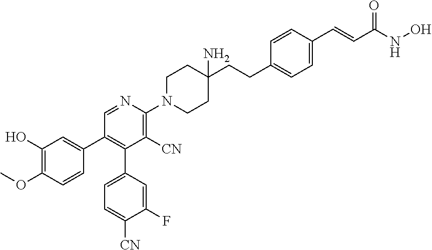 [SMILES: COc1ccc(-c2cnc(N3CCC(N)(CCc4ccc(/C=C/C(=O)NO)cc4)CC3)c(C#N)c2-c2ccc(C#N)c(F)c2)cc1O]

¹H NMR (400 MHZ, DMSO-d₆) δ 10.67 (s, 1H), 9.01 (s, 1H), 8.44 (s, 1H), 8.20 (s, 1H), 7.96 (dd, J=8.0, 6.9 Hz, 1H), 7.67 (dd, J=10.0, 1.4 Hz, 1H), 7.49 (d, J=7.8 Hz, 2H), 7.42 (d, J=15.8 Hz, 1H), 7.34-7.24 (m, 3H), 6.85-6.76 (m, 1H), 6.53-6.37 (m, 3H), 3.78 (d, J=5.2 Hz, 4H), 3.72 (s, 3H), 2.77-2.65 (m, 2H), 1.93-1.71 (m, 6H).

ESI-MS (m/z)=633.3 [M+H]⁺.

### Example 88

### Preparation of (E)-3-(4-(((1-(4-(4-cyano-3-fluorophenyl)-5-(3-hydroxy-4-methoxyphenyl)-3-methoxypyridin-2-yl)piperidin-4-yl)amino)methyl)phenyl)-N-hydroxyacrylamide

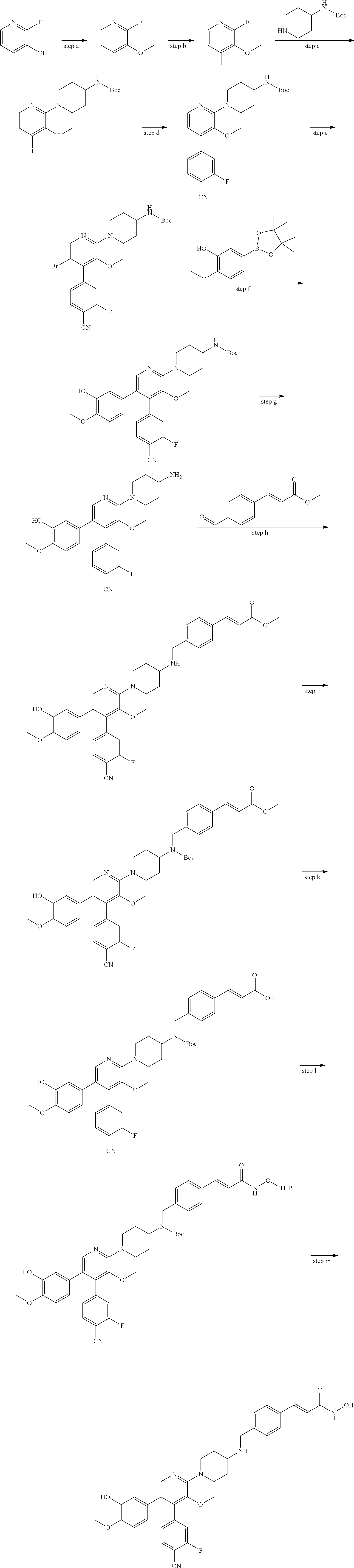

### Step a): preparation of 2-fluoro-3-methoxypyridine

2-Fluoropyridin-3-ol (3.0 g, 26.5 mmol) and potassium carbonate (11.0 g, 79.6 mmol) were mixed in DMF (30 mL), and methyl iodide (7.6 g, 53.1 mmol) was slowly added to the solution. After the addition, the mixture was stirred at room temperature for 6 hours. Water (200 mL) was added, and the mixture was extracted with ethyl acetate (50 mL×3). The organic layer was washed with saturated brine (100 mL), dried over anhydrous sodium sulfate, filtered, and concentrated under vacuum. The residue was purified by silica gel chromatography (eluent: petroleum ether/ethyl acetate=5:1) to afford 2-fluoro-3-methoxypyridine with a yield of 75.5%.

ESI-MS m/z=128.1 [M+H]⁺.

### Step b): preparation of 2-fluoro-4-iodo-3-methoxypyridine

2-Fluoro-3-methoxypyridine (1.2 g, 8.4 mmol) was dissolved in anhydrous tetrahydrofuran (50 mL), and the reaction solution was purged with nitrogen three times, and cooled to −78° C. n-Butyllithium (12.7 mmol) was slowly added to the reaction liquid, stirred at −78° C. for 30 minutes. Iodine (3.2 g, 12.5 mmol) in anhydrous THF (20 mL) was slowly added to the reaction solution. After the addition, the reaction was stirred at room temperature for another 1 hour. Saturated ammonium chloride solution (100 mL) was added, and the mixture was extracted with ethyl acetate (30 mL×3). The organic layers were combined, washed with brine (20 mL), dried over anhydrous sodium sulfate, filtered, and concentrated under vacuum. The residue was purified by silica gel chromatography (eluent: petroleum ether/ethyl acetate=5/1) to afford 2-fluoro-4-iodo-3-methoxypyridine with a yield of 51.4%. ESI-MS m/z=254.1 [M+H]⁺.

### Step c): preparation of tert-butyl (1-(4-iodo-3-methoxypyridin-2-yl)piperidin-4-yl)carbamate

2-Fluoro-4-iodo-3-methoxypyridine (1.1 g, 4.4 mmol) and 4-tert-butoxycarbonylpiperazine (1.3 g, 6.5 mmol) were dissolved in DMF (20 mL), then N-methylmorpholine (2.2 g, 21.8 mmol) was added, and the reaction solution was heated to 80° C. and stirred overnight. Water (100 mL) was added, and the mixture was extracted with ethyl acetate (40 mL×3). The organic layers were combined, washed with brine (20 mL), dried over anhydrous sodium sulfate, filtered, and concentrated under vacuum. The residue was purified by silica gel chromatography (eluent: petroleum ether/ethyl acetate=6:1) to afford tert-butyl (1-(4-iodo-3-methoxypyridin-2-yl)piperidin-4-yl)carbamate with a yield of 48.8%.

ESI-MS m/z=434.0 [M+H]⁺.

### Step d): preparation of tert-butyl (1-(4-(4-cyano-3-fluorophenyl)-3-methoxypyridin-2-yl)piperidin-4-yl)carbamate

Tert-butyl (1-(4-iodo-3-methoxypyridin-2-yl)piperidin-4-yl)carbamate (1.0 g, 2.3 mmol), 4-cyano-3-fluorophenylboronic acid (400 mg, 2.4 mmol), sodium carbonate (730 mg, 6.9 mmol), and [1,1′-bis(diphenylphosphino) ferrocene]palladium dichloride (85 mg, 0.1 mmol) were dissolved in a mixture of 1,4-dioxane (20 mL) and water (5.0 mL), and the reactionsolution was heated to 100° C. and stirred for 2 hours. The reactionsolution was concentrated by rotary evaporation, and the residue was purified by silica gel chromatography (eluent: petroleum ether/ethyl acetate=3:1) to afford tert-butyl (1-(4-(4-cyano-3-fluorophenyl)-3-methoxypyridin-2-yl)piperidin-4-yl)carbamate with a yield of 63.8%.

ESI-MS m/z=427.4 [M+H]⁺.

Step e to Step 1 were Similar to Step c to Step i in Example 84.

### Step m): preparation of (E)-3-(4-((1-(4-(4-cyano-3-fluorophenyl)-5-(3-hydroxy-4-methoxyphenyl)-3-methoxypyridin-2-yl)piperidin-4-yl)amino)methyl)phenyl)-N-hydroxyacrylamide formate

Tert-butyl (E)-(1-(4-(4-cyano-3-fluorophenyl)-5-(3-hydroxy-4-methoxyphenyl)-3-methoxypyridin-2-yl)piperidin-4-yl)(4-(3-oxo-3-((tetrahydro-2H-pyran-2-yl)oxy)amino)prop-1-en-1-yl)benzyl)carbamate (80 mg, 0.19 mmol) was dissolved in a solution of 4 M HCl in ethyl acetate (5 mL) and the mixture was reacted at room temperature for 1 hour. After the reaction was completed, the reaction solution was concentrated with rotary evaporation, and the residue was purified by Prep-HPLC (separation method 3) to afford (E)-3-(4-((1-(4-(4-cyano-3-fluorophenyl)-5-(3-hydroxy-4-methoxyphenyl)-3-methoxypyridin-2-yl)piperidin-4-yl)amino)methyl)phenyl)-N-hydroxyacrylamide formate with a yield of 40.4%.

¹H NMR (400 MHZ, DMSO-d₆) δ 10.72 (s, 1H), 8.92 (s, 2H), 7.98 (s, 1H), 7.85 (t, J=7.6 Hz, 1H), 7.52 (d, J=8.0 Hz, 2H), 7.48-7.37 (m, 4H), 7.18-7.08 (m, 1H), 6.77 (d, J=8.8 Hz, 1H), 6.49-6.40 (m, 3H), 3.99 (d, J=12.8 Hz, 2H), 3.84 (s, 2H), 3.70 (s, 3H), 3.46 (s, 3H), 2.86 (t, J=11.6 Hz, 2H), 2.68 (s, 1H), 2.06-1.96 (m, 2H), 1.48 (q, J=10.8 Hz, 2H).

ESI-MS m/z: 624.3 [M+H]⁺.

### Example 89

### Preparation of (E)-3-(4-(2-(1-(3-cyano-4-(4-cyano-3-fluorophenyl)-5-(3-hydroxy-4-methoxyphenyl)pyridin-2-yl)piperidin-4-yl)ethyl)phenyl)-N-hydroxyacrylamide

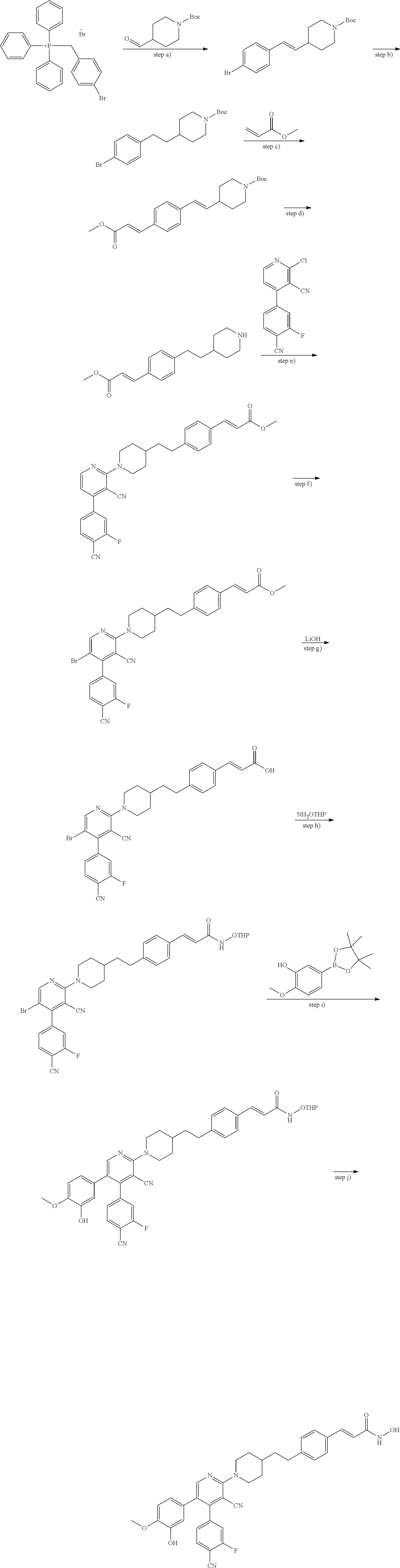

### Step a): Preparation of tert-butyl (E)-4-(4-bromostyryl)piperidine-1-carboxylate

(4-Bromobenzyl)triphenylphosphonium bromide (2.0 g, 3.8 mmol), NaHMDS (700 mg, 3.8 mmol), and ACN (20 mL) were added to a reaction flask, and the mixture was stirred at room temperature for 8 hours, then tert-butyl 4-formylpiperidine-1-carboxylate (808 mg, 3.8 mmol) was added, and the mixture was stirred overnight. After the reaction was completed, water (20 mL) was added to quench the reaction, and the mixture was extracted with ethyl acetate (20 mL×2). The organic phases were combined, washed with saturated brine (20 mL×2), dried over anhydrous sodium sulfate, filtered, and the residue was purified by silica gel chromatography (eluent: petroleum ether/ethyl acetate=3/1) to afford tert-butyl (E)-4-(4-bromostyryl)piperidine-1-carboxylate with a yield of 91%.

ESI-MS m/z=366.1 [M+H]⁺.

### Step b): preparation of tert-butyl 4-(4-bromophenyl)piperidine-1-carboxylate

Tert-butyl (E)-4-(4-bromostyryl)piperidine-1-carboxylate (1.3 g, 3.4 mmol), palladium on carbon (150 mg, 5%) and ethanol (5 mL) were sequentially added to a reaction flask, and the mixture was purged with hydrogen three times, and then stirred under a hydrogen atmosphere at room temperature for 2 hours. After the reaction was completed, the mixture was filtered, and the filtrate was concentrated under reduced pressure to afford tert-butyl 4-(4-bromophenyl)piperidine-1-carboxylate with a yield of 92.0%.

ESI-MS (m/z)=368.1 [M+H]⁺.

### Step c): preparation of tert-butyl (E)-4-(4-(3-methoxy-3-oxoprop-1-en-1-yl)phenethyl)piperidine-1-carboxylate

Tert-butyl 4-(4-bromophenyl)piperidine-1-carboxylate (1.2 g, 3.2 mmol), methyl acrylate (535 mg, 6.4 mmol), Cs₂CO₃ (2.1 g, 6.4 mmol), Pd(dppf)Cl₂ (117 mg, 0.16 mmol), 1,4-dioxane (12 mL), and H₂O (4 mL) were added to a reaction flask, and the mixture was stirred at 100° C. for 1 hour. The mixture was concentrated to dryness under reduced pressure, and the residue was then purified by silica gel chromatography (eluent: petroleum ether/ethyl acetate=1/1) to afford tert-butyl (E)-4-(4-(3-methoxy-3-oxoprop-1-en-1-yl)phenethyl)piperidine-1-carboxylate with a yield of 72.5%.

ESI-MS (m/z)=374.2 [M+H]⁺.

### Step d): preparation of methyl (E)-3-(4-(2-(piperidin-4-yl)ethyl)phenyl)acrylate

Tert-butyl (E)-4-(4-(3-methoxy-3-oxoprop-1-en-1-yl)phenethyl)piperidine-1-carboxylate (868 mg, 2.3 mmol) was added to a reaction flask, followed by adding hydrogen chloride solution in ethyl acetate (4M, 5 mL), and the mixture was stirred at room temperature for 1 hour. The mixture was concentrated under reduced pressure to afford methyl (E)-3-(4-(2-(piperidin-4-yl)ethyl)phenyl)acrylate with a yield of 98.5%.

ESI-MS (m/z)=274.2 [M+H]⁺.

### Step e): preparation of methyl (E)-3-(4-(2-(1-(3-cyano-4-(4-cyano-3-fluorophenyl)pyridin-2-yl)piperidin-4-yl)ethyl)phenyl)acrylate

2-Chloro-4-(4-cyano-3-fluorophenyl) nicotinonitrile (500 mg, 1.9 mmol), methyl (E)-3-(4-(2-(piperidin-4-yl)ethyl)phenyl)acrylate (521 mg, 1.9 mmol), and triethylamine (2 mL) were added to a sealed reaction tube containing ACN (20 mL), and the mixture was stirred at 70° C. for 8 hours. After the reaction was completed, water (20 mL) was added to quench the reaction, followed by extraction with ethyl acetate (20 mL×2). The organic phases were combined, washed with saturated brine (20 mL×2), dried with anhydrous sodium sulfate, filtered, and the residue was purified by silica gel chromatography (eluent: petroleum ether/ethyl acetate=2/1) to afford methyl (E)-3-(4-(2-(1-(3-cyano-4-(4-cyano-3-fluorophenyl)pyridin-2-yl)piperidin-4-yl)ethyl)phenyl)acrylate with a yield of 71%.

ESI-MS m/z=495.2 [M+H]⁺.

### Step f): preparation of methyl (E)-3-(4-(2-(1-(5-bromo-3-cyano-4-(4-cyano-3-fluorophenyl)pyridin-2-yl)piperidin-4-yl)ethyl)phenyl)acrylate

Methyl (E)-3-(4-(2-(1-(3-cyano-4-(4-cyano-3-fluorophenyl)pyridin-2-yl)piperidin-4-yl)ethyl)phenyl)acrylate (693 mg, 1.4 mmol) and ACN (10 mL) were added to a reaction flask, then NBS (299 mg, 1.7 mmol) was added dropwise under stirring in an ice bath, and the reaction was continued under stirring in the ice bath for 2 hours. After the reaction was completed, saturated sodium bicarbonate aqueous solution (40 mL) was added, and the mixture was extracted with ethyl acetate (40 mL×3). The organic phases were combined, washed with saturated brine (40 mL×2), and then concentrated to dryness under reduced pressure. The residue was purified by silica gel chromatography (eluent: petroleum ether/ethyl acetate=1/1) to afford methyl (E)-3-(4-(2-(1-(5-bromo-3-cyano-4-(4-cyano-3-fluorophenyl)pyridin-2-yl)piperidin-4-yl)ethyl)phenyl)acrylate with a yield of 57.0%. ESI-MS (m/z)=573.1 [M+H]⁺.

### Step g): preparation of (E)-3-(4-(2-(1-(5-bromo-3-cyano-4-(4-cyano-3-fluorophenyl)pyridin-2-yl)piperidin-4-yl)ethyl)phenyl)acrylic acid

Methyl (E)-3-(4-(2-(1-(5-bromo-3-cyano-4-(4-cyano-3-fluorophenyl)pyridin-2-yl)piperidin-4-yl)ethyl)phenyl)acrylate (457 mg, 798 μmol) and lithium hydroxide (168 mg, 4.0 mmol) were added to a reaction flask containing THF (5 mL), MeOH (3 mL), and water (2 mL), and the mixture was stirred at room temperature for 2 hours. 2M HCl aqueous solution was added dropwise to adjust the pH to 4, and the mixture was then extracted with a mixture of MeOH/DCM (1:5) (20 mL×2). The organic phases were combined, washed with saturated brine (15 mL×2), dried over anhydrous sodium sulfate, and concentrated to afford (E)-3-(4-(2-(1-(5-bromo-3-cyano-4-(4-cyano-3-fluorophenyl)pyridin-2-yl)piperidin-4-yl)ethyl)phenyl)acrylic acid, which was directly used in the next step.

ESI-MS (m/z)=559.1 [M+H]⁺.

### Step h): preparation of (E)-3-(4-(2-(1-(5-bromo-3-cyano-4-(4-cyano-3-fluorophenyl)pyridin-2-yl)piperidin-4-yl)ethyl)phenyl)-N-(tetrahydro-2H-pyran-2-yl)oxy)acrylamide

(E)-3-(4-(2-(1-(5-Bromo-3-cyano-4-(4-cyano-3-fluorophenyl)pyridin-2-yl)piperidin-4-yl)ethyl)phenyl)acrylic acid (446 mg, 798 μmol), O-(tetrahydro-2H-pyran-2-yl) hydroxylamine (187 mg, 1.6 mmol), HATU (364 mg, 957 μmol), and DIEA (205 mg, 1.6 mmol) were added to a reaction flask containing DMF (5 mL), and the mixture was stirred at room temperature for 2 hours. Water (10 mL) was added to quench the reaction, and the mixture was extracted with ethyl acetate (20 mL×2). The organic phases were combined, washed with saturated brine (15 mL×2), dried over anhydrous sodium sulfate, and concentrated under vacuum, and the residue was purified by silica gel chromatography (eluent: DCM/MeOH=10/1) to afford (E)-3-(4-(2-(1-(5-bromo-3-cyano-4-(4-cyano-3-fluorophenyl)pyridin-2-yl)piperidin-4-yl)ethyl)phenyl)-N-(tetrahydro-2H-pyran-2-yl)oxy)acrylamide with a yield of 47%.

ESI-MS m/z=658.2 [M+H]⁺.

### Step i): preparation of (E)-3-(4-(2-(1-(3-cyano-4-(4-cyano-3-fluorophenyl)-5-(3-hydroxy-4-methoxyphenyl)pyridin-2-yl)piperidin-4-yl)ethyl)phenyl)-N-(tetrahydro-2H-pyran-2-yl)oxy)acrylamide

(E)-3-(4-(2-(1-(5-Bromo-3-cyano-4-(4-cyano-3-fluorophenyl)pyridin-2-yl)piperidin-4-yl)ethyl)phenyl)-N-(tetrahydro-2H-pyran-2-yl)oxy)acrylamide (247 mg, 375 μmol), (3-hydroxy-4-methoxyphenyl) boronic acid (113 mg, 450 μmol), Cs₂CO₃ (245 mg, 750 μmol), Pd(dppf)Cl₂ (27 mg, 37.5 μmol), 1,4-dioxane (4 mL), and H₂O (1 mL) were added to a reaction flask, and the mixture was stirred at 90° C. for 1 hour. The mixture was concentrated to dryness under reduced pressure, and the residue was purified by silica gel chromatography (eluent: petroleum ether/ethyl acetate=1/3) to afford (E)-3-(4-(2-(1-(3-cyano-4-(4-cyano-3-fluorophenyl)-5-(3-hydroxy-4-methoxyphenyl)pyridin-2-yl)piperidin-4-yl)ethyl)phenyl)-N-(tetrahydro-2H-pyran-2-yl)oxy)acrylamide with a yield of 42.5%.

ESI-MS (m/z)=702.3 [M+H]⁺.

### Step j): preparation of (E)-3-(4-(2-(1-(3-cyano-4-(4-cyano-3-fluorophenyl)-5-(3-hydroxy-4-methoxyphenyl)pyridin-2-yl)piperidin-4-yl)ethyl)phenyl)-N-hydroxyacrylamide

(E)-3-(4-(2-(1-(3-Cyano-4-(4-cyano-3-fluorophenyl)-5-(3-hydroxy-4-methoxyphenyl)pyridin-2-yl)piperidin-4-yl)ethyl)phenyl)-N-(tetrahydro-2H-pyran-2-yl)oxy)acrylamide (112 mg, 159 μmol) was added to a reaction flask, followed by adding hydrogen chloride solution in ethyl acetate (4M, 2.5 mL), and the mixture was stirred at room temperature for 1 hour, resulting in the precipitation of a significant amount of solid. The mixture was concentrated under reduced pressure, and the obtained crude product was purified by Prep-HPLC (separation method 1) to afford (E)-3-(4-(2-(1-(3-cyano-4-(4-cyano-3-fluorophenyl)-5-(3-hydroxy-4-methoxyphenyl)pyridin-2-yl)piperidin-4-yl)ethyl)phenyl)-N-hydroxyacrylamide with a yield of 11.5%.

¹H NMR (400 MHZ, DMSO-d₆) δ 10.71 (s, 1H), 8.99 (s, 1H), 8.52 (s, 1H), 8.41 (s, 1H), 7.95 (t, J=7.4 Hz, 1H), 7.66 (d, J=10.0 Hz, 1H), 7.53-7.38 (m, 3H), 7.27 (d, J=7.8 Hz, 3H), 6.80 (d, J=8.0 Hz, 1H), 6.52-6.33 (m, 3H), 4.27 (d, J=12.8 Hz, 2H), 3.71 (s, 3H), 3.03 (t, J=12.0 Hz, 2H), 2.67 (t, J=7.4 Hz, 2H), 1.87 (d, J=5.2 Hz, 2H), 1.56 (q, J=10.0, 8.8 Hz, 3H), 1.30 (d, J=5.2 Hz, 2H).

ESI-MS (m/z)=618.2 [M+H]⁺.

### Example 90

### Preparation of (E)-3-(4-(((1-(3-cyano-4-(4-cyano-3-fluorophenyl)-5-(3-hydroxy-4-(E)-3-(hydroxyamino)-3-oxoprop-1-en-1-yl)phenyl)pyridin-2-yl)piperidin-4-yl)amino)methyl)phenyl)-N-hydroxyacrylamide

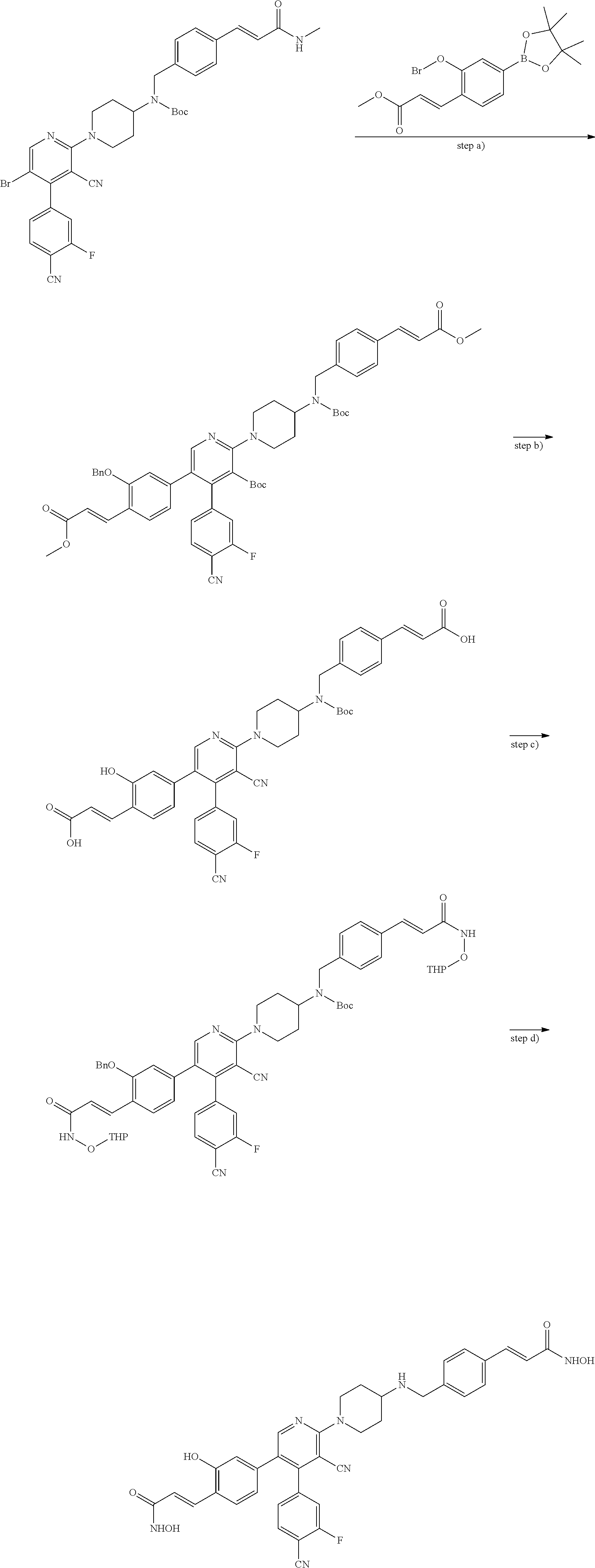

### Step a): preparation of tert-butyl 5-(3-(benzyloxy)-4-((E)-3-methoxy-3-oxoprop-1-en-1-yl)phenyl)-2-(4-(tert-butoxycarbonyl)(4-(E)-3-methoxy-3-oxoprop-1-en-1-yl)benzyl)amino)piperidin-1-yl)-4-(4-cyano-3-fluorophenyl) nicotinate

Methyl (E)-3-(4-(((1-(5-bromo-3-cyano-4-(4-cyano-3-fluorophenyl)pyridin-2-yl)piperidin-4-yl) (tert-butoxycarbonyl)amino)methyl)phenyl)acrylate (200 mg, 297 μmol), methyl (E)-3-(2-(benzyloxy)-4-(4,4,5,5-tetramethyl-1,3,2-dioxaborolan-2-yl)phenyl)acrylate (182 mg, 446 μmol), Cs₂CO₃ (193 mg, 594 μmol), Pd(dppf)Cl₂ (22 mg, 30 μmol), 1,4-dioxane (4 mL), and H₂O (1 mL) were added to a reaction flask, and the mixture was stirred at 120° C. for 1 hour, concentrated under reduced pressure. And the residue was purified by silica gel chromatography (eluent: petroleum ether/ethyl acetate=1/1) to afford tert-butyl 5-(3-(benzyloxy)-4-((E)-3-methoxy-3-oxoprop-1-en-1-yl)phenyl)-2-(4-(tert-butoxycarbonyl)(4-(E)-3-methoxy-3-oxoprop-1-en-1-yl)benzyl)amino)piperidin-1-yl)-4-(4-cyano-3-fluorophenyl) nicotinate with a yield of 78.5%. ESI-MS (m/z)=938.1 [M+H]⁺.

### Step b): preparation of (E)-3-(4-(1-(5-(3-(benzyloxy)-4-(2-carboxyvinyl)phenyl)-3-cyano-4-(4-cyano-3-fluorophenyl)pyridin-2-yl)piperidin-4-yl) (tert-butoxycarbonyl)amino)methyl)phenyl)acrylic acid

Tert-butyl 5-(3-(benzyloxy)-4-((E)-3-methoxy-3-oxoprop-1-en-1-yl)phenyl)-2-(4-(tert-butoxycarbonyl)(4-(E)-3-methoxy-3-oxoprop-1-en-1-yl)benzyl)amino)piperidin-1-yl)-4-(4-cyano-3-fluorophenyl) nicotinate (219 mg, 233 μmol), lithium hydroxide monohydrate (50 mg, 1.2 mmol), tetrahydrofuran (2 mL), isopropanol (2 mL), and water (1 mL) were added to a reaction flask, and the mixture was stirred at room temperature for 12 hours. Under stirring in an ice bath, 1N concentrated hydrochloric acid was slowly added to adjust the pH to 3-4, then water (10 mL) was then added, and the mixture was extracted with ethyl acetate (10 mL×3). The organic phases were combined, washed with saturated brine (10 mL×2), and concentrated to dryness under reduced pressure to afford (E)-3-(4-(1-(5-(3-(benzyloxy)-4-(2-carboxyvinyl)phenyl)-3-cyano-4-(4-cyano-3-fluorophenyl)pyridin-2-yl)piperidin-4-yl) (tert-butoxycarbonyl)amino)methyl)phenyl)acrylic acid which was used directly for the next reaction step. ESI-MS (m/z)=834.4 [M+H]⁺.

### Step c): preparation of tert-butyl (1-(5-(3-(benzyloxy)-4-((E)-3-oxo-3-(((tetrahydro-2H-pyran-2-yl)oxy)amino)prop-1-en-1-yl)phenyl)-3-cyano-4-(4-cyano-3-fluorophenyl)pyridin-2-yl)piperidin-4-yl)(4-((E)-3-oxo-3-(((tetrahydro-2H-pyran-2-yl)oxy)amino)prop-1-en-1-yl)benzyl)carbamate

(E)-3-(4-(1-(5-(3-(Benzyloxy)-4-(2-carboxyvinyl)phenyl)-3-cyano-4-(4-cyano-3-fluorophenyl)pyridin-2-yl)piperidin-4-yl) (tert-butoxycarbonyl)amino)methyl)phenyl)acrylic acid (194 mg, 233 μmol), O-(tetrahydro-2H-pyran-2-yl) hydroxylamine (55 mg, 466 μmol), DIEA (90 mg, 699 μmol), and DMF (2 mL) were added to a reaction flask, then HATU (133 mg, 350 μmol) was added under stirring at room temperature, and the solution was stirred for 1 hour at room temperature. After the reaction was completed, water (10 mL) was added to quench the reaction, followed by extraction with ethyl acetate (20 mL×2). And the organic phases were combined and washed sequentially with saturated sodium bicarbonate aqueous solution (20 mL×1) and saturated brine (10 mL×2), dried over anhydrous sodium sulfate, and filtered. The filtrate was concentrated under reduced pressure, and the residue was purified by silica gel chromatography (eluent: petroleum ether/ethyl acetate=1/1) to afford tert-butyl (1-(5-(3-(benzyloxy)-4-(E)-3-oxo-3-(tetrahydro-2H-pyran-2-yl)oxy)amino)prop-1-en-1-yl)phenyl)-3-cyano-4-(4-cyano-3-fluorophenyl)pyridin-2-yl)piperidin-4-yl)(4-((E)-3-oxo-3-((tetrahydro-2H-pyran-2-yl)oxy)amino)prop-1-en-1-yl)benzyl)carbamate with a yield of 60.5%.

ESI-MS (m/z)=1033.2 [M+H]⁺.

### Step d): preparation of (E)-3-(((4-(1-(3-cyano-4-(4-cyano-3-fluorophenyl)-5-(3-hydroxy-4-(E)-3-(hydroxyamino)-3-oxoprop-1-en-1-yl)phenyl)pyridin-2-yl)piperidin-4-yl)amino)methyl)phenyl)-N-hydroxyacrylamide

Tert-butyl (1-(5-(3-(benzyloxy)-4-((E)-3-oxo-3-(((tetrahydro-2H-pyran-2-yl)oxy)amino)prop-1-en-1-yl)phenyl)-3-cyano-4-(4-cyano-3-fluorophenyl)pyridin-2-yl)piperidin-4-yl)(4-((E)-3-oxo-3-(((tetrahydro-2H-pyran-2-yl)oxy)amino)prop-1-en-1-yl)benzyl)carbamate (145 mg, 141 μmol) was added to a reaction flask, followed by hydrogen chloride solution in ethyl acetate (4M, 2.5 mL), and the mixture was stirred at room temperature for 1 hour, resulting in the precipitation of a significant amount of solid. After concentrated under reduced pressure, the resultant crude product was purified by Prep-HPLC (separation method 3) to afford (E)-3-(((4-(1-(3-cyano-4-(4-cyano-3-fluorophenyl)-5-(3-hydroxy-4-(E)-3-(hydroxyamino)-3-oxoprop-1-en-1-yl)phenyl)pyridin-2-yl)piperidin-4-yl)amino)methyl)phenyl)-N-hydroxyacrylamide formate with a yield of 20.5%. ¹H NMR (400 MHZ, Methanol-d₄) δ8.52 (s, 1H), 8.46 (s, 1H), 7.81-7.69 (m, 2H), 7.67-7.49 (m, 5H), 7.43 (dd, J=9.6, 1.4 Hz, 1H), 7.33 (d, J=8.4 Hz, 1H), 7.22 (dd, J=8.0, 1.4 Hz, 1H), 6.64-6.45 (m, 4H), 4.48 (d, J=13.0 Hz, 2H), 4.21 (s, 2H), 3.55 (s, 1H), 3.19 (t, J=12.6 Hz, 2H), 2.37-2.21 (m, 2H), 1.80 (td, J=13.0, 9.0 Hz, 2H).

ESI-MS (m/z)=674.2 [M+H]⁺.

Examples 91 to 174 were prepared similarly according to the synthetic method of Example 37 (the separation method for the compounds: hydrochloride, trifluoroacetate, and formate were prepared according to separation methods 1, 2, and 3, respectively), and the structure and characterization data are as follows:

| | | | | MS |
|---|---|---|---|---|
| Example | Chemical name | Structure | ¹H NMR | (M + H)⁺ |
| 91 | 4-(4-(1-(3-Cyano- 4-(4-cyano-3- fluorophenyl)-5- (4-hydroxy-3- methoxyphenyl) pyridin-2- yl)piperidin-4- yl)amino)methyl) piperidin-1-yl)-N- hydroxybenzamide formate | 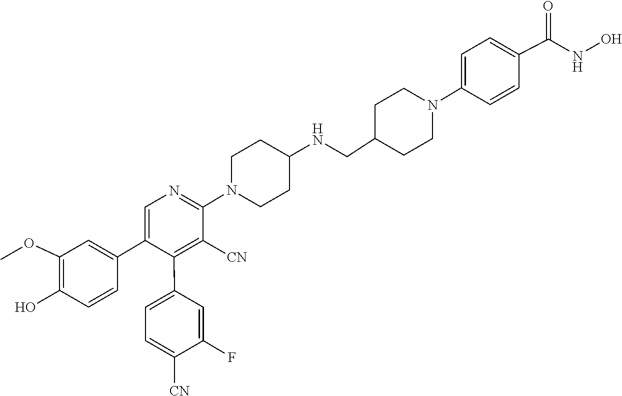 [SMILES: COc1cc(-c2cnc(N3CCC(NCC4CCN(c5ccc(C(=O)NO)cc5)CC4)CC3)c(C#N)c2-c2ccc(C#N)c(F)c2)ccc1O] | ¹H NMR (400 MHz, Methanol- d4)δ ppm 8.52 (s, 1.4H), 8.47 (s, 1H), 7.75 (dd, J = 8.0, 6.6 Hz, 1H), 7.64 (d, J = 8.8 Hz, 2H), 7.37 (dd, J = 9.8, 1.4 Hz, 1H), 7.26 (dd, J = 8.0, 1.4 Hz, 1H), 6.97 (d, J = 8.8 Hz, 2H), 6.77-6.63 (m, 1H), 6.62- 6.47 (m, 2H), 4.42 (d, J = 13.4 Hz, 2H), 3.92 (d, J = 12.6 Hz, 2H), 3.64 (s, 3H), 3.16 (t, J = 12.2 Hz, 3H), 3.00-2.74 (m, 4H), 2.34- 2.13 (m, 2H), 1.90 (d, J = 12.4 Hz, 3H), 1.76 (q, J = 2.0, 5.2 Hz, 2H), 1.41 (q, J = 9.8 Hz, 2H). | 676.3 |
| 92 | (E)-3-(3-(2-(1-(3- Cyano-4-(4- cyano-3- fluorophenyl)-5- (4-hydroxy-3- methoxyphenyl) pyridin-2- yl)piperidin-4- yl)amino)ethyl) phenyl)-N- hydroxyacrylamide formate | 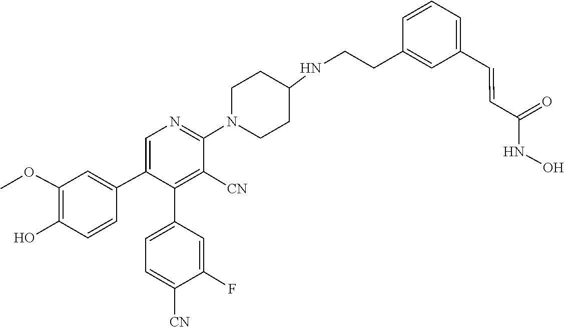 [SMILES: COc1cc(-c2cnc(N3CCC(NCCc4cccc(/C=C/C(=O)NO)c4)CC3)c(C#N)c2-c2ccc(C#N)c(F)c2)ccc1O] | ¹H NMR (400 MHz, Methanol- d4)δ ppm: 8.51 (s, 1H), 8.42 (s, 1H), 7.74 (dd, J = 8.0, 6.4 Hz, 1H), 7.60-7.42 (m, 3H), 7.42-7.27 (m, 3H), 7.22 (dd, J = 8.0, 1.6 Hz, 1H), 6.86-6.78 (m, 1H), 6.50 (m, 3H), 4.40 (d, J = 13.6 Hz, 2H), 3.81 (s, 3H), 3.29-3.07 (m, 5H), 2.98 (t, J = 8.0 Hz, 2H), 2.19 (d, J = 12.4 Hz, 2H), 1.73 (m, 2H). | 633.2 |
| 93 | 4-(3-(3-Cyano-4- (4-cyano-3- fluorophenyl)-5- (4-hydroxy-3- methoxyphenyl) pyridin-2- yl)piperidin-4- yl)amino)propyl)- N- hydroxybenzamide formate | 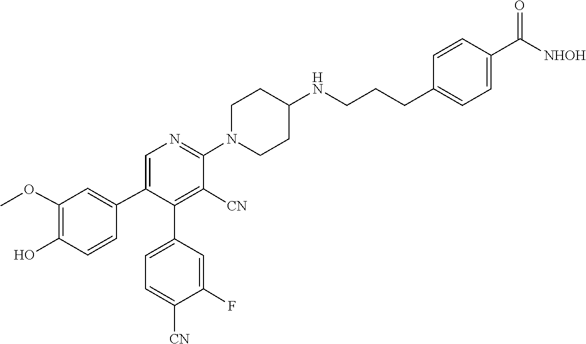 | ¹H NMR (400 MHz, DMSO-d6) δ ppm: 8.49 (s, 1H), 8.30 (s, 1H), 7.96 (dd, J = 8.0, 6.8 Hz, 1H), 7.74- 7.58 (m, 3H), 7.35-7.24 (m, 3H), 6.66 (d, J = 8.0 Hz, 1H), 6.57 (m, 1H), 6.48 (dd, J = 8.0, 2.0 Hz, 1H), 4.27-4.13 (m, 2H), 3.55 (s, 3H), 3.16 (t, J = 12.2 Hz, 2H), 2.80 (s, 1H), 2.66 (m, 4H), 1.97 (d, J = 12.8 Hz, 2H), 1.84-1.69 (m, 2H), 1.43 (m, 2H). | 621.3 |
| 94 | (E)-3-(4-(2-(3- Cyano-4-(4- cyano-3- fluorophenyl)-5- (4-hydroxy-3- methoxyphenyl) pyridin-2-yl)-2- azabicyclo[2.2.1] heptan-5- yl)amino)methyl) phenyl)-N- hydroxyacrylamide formate | 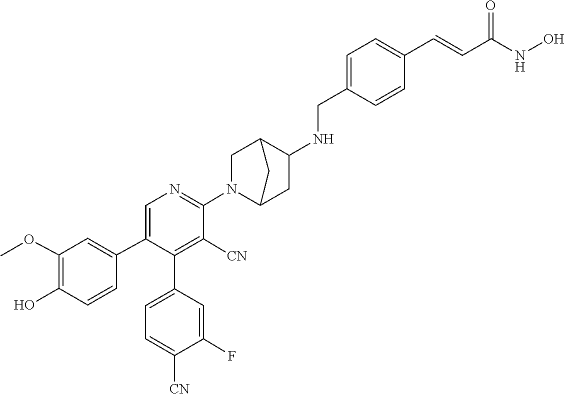 [SMILES: COc1cc(-c2cnc(N3CC4CC3CC4NCc3ccc(/C=C/C(=O)NO)cc3)c(C#N)c2-c2ccc(C#N)c(F)c2)ccc1O] | ¹H NMR (400 MHz, Methanol-d4) δ 8.52 (s, 1H), 8.32 (d, J = 3.8 Hz, 1H), 7.73 (s, 1H), 7.56 (m, 3H), 7.48-7.33 (m, 3H), 7.24 (s, 1H), 6.67 (d, J = 8.2 Hz, 1H), 6.53- 6.43 (m, 3H), 4.02-3.83 (m, 4H), 3.63 (m, 3H),3.58 (s, 1H), 3.08 (s, 1H), 2.84 (d, J = 10.8 Hz, 1H), 2.31 (t, J = 10.6 Hz, 0.5H), 2.18 (t, J = 11.2 Hz, 0.5H), 1.96-1.85 (m, 1H), 1.78-1.70 (m, 1H), 1.63-1.51 (m, 1H). | 631.3 |
| 95 | (E)-3-(6-((1-(3- Cyano-4-(4- cyano-3- fluorophenyl)-5- (4-hydroxy-3- methoxyphenyl) pyridin-2- yl)piperidin-4- yl)amino)methyl) pyridin-3-yl)-N- hydroxyacrylamide formate | 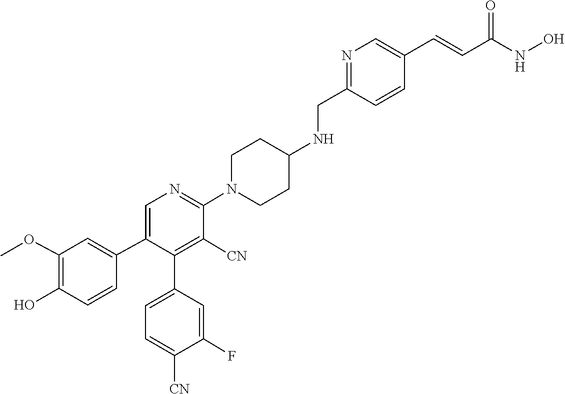 [SMILES: COc1cc(-c2cnc(N3CCC(NCc4ccc(/C=C/C(=O)NO)cn4)CC3)c(C#N)c2-c2ccc(C#N)c(F)c2)ccc1O] | ¹H NMR (400 MHz, DMSO-d6) δ 10.80 (s, 1H), 9.09 (s, 1H), 8.69 (d, J = 2.4 Hz, 1H), 8.49 (s, 1H), 8.15 (s, 1H), 8.02-7.88 (m, 2H), 7.63 (dd, J = 10.0, 1.2 Hz, 1H), 7.59- 7.42 (m, 2H), 7.31 (dd, J = 8.0, 1.6 Hz, 1H), 6.65 (d, J = 8.0 Hz, 1H), 6.60-6.42 (m, 3H), 4.18 (d, J = 13.2 Hz, 2H), 3.95 (s, 2H), 3.55 (s, 3H), 3.17 (t, J = 12.0 Hz, 2H), 2.80 (s, 1H), 2.01 (d, J = 12.8 Hz, 2H), 1.48 (q, J = 10.8, 10.0 Hz, 2H). | 620.2 |
| 96 | (E)-3-(5-((1-(3- Cyano-4-(4- cyano-3- fluorophenyl)-5- (4-hydroxy-3- methoxyphenyl) pyridin-2- yl)piperidin-4- yl)amino)methyl) pyrimidin-2-yl)-N- hydroxyacrylamide formate | 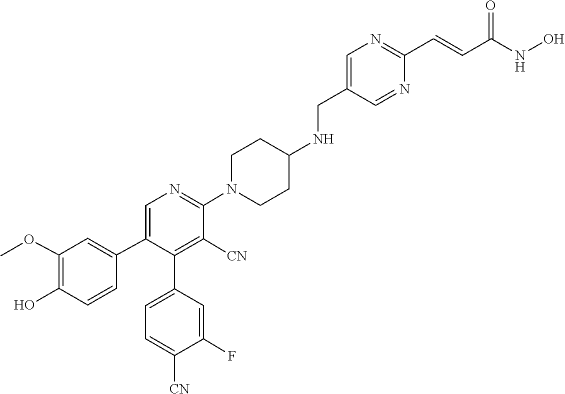 [SMILES: COc1cc(-c2cnc(N3CCC(NCc4cnc(/C=C/C(=O)NO)nc4)CC3)c(C#N)c2-c2ccc(C#N)c(F)c2)ccc1O] | ¹H NMR (400 MHz, Methanol- d4) δ 8.85 (s, 1H), 8.44 (s, 1H),8.41 (s, 1H), 7.75 (dd, J = 8.0, 6.8 Hz, 1H), 7.56 (d, J = 15.6 Hz, 1H), 7.37 (dd, J = 9.6, 1.6 Hz, 1H), 7.29-7.17 (m, 2H), 6.70 (d, J = 8.4 Hz, 1H), 6.55 (d, J = 6.8 Hz, 2H), 4.38 (d, J = 13.2 Hz, 2H), 4.03 (s, 2H), 3.64 (s, 3H), 3.18 (t, J = 12.4 Hz, 2H), 3.01 (d, J = 11.2 Hz, 1H), 2.27-2.10 (m, 2H), 1.68 (t, J = 11.6 Hz, 2H). | 621.2 |
| 97 | (E)-3-(4-((1-(3- Cyano-4-(4- cyano-3- fluorophenyl)-5- (4-fluoro-3- hydroxyphenyl) pyridin-2- yl)piperidin-4- yl)amino)methyl) phenyl)-N- hydroxyacrylamide formate | 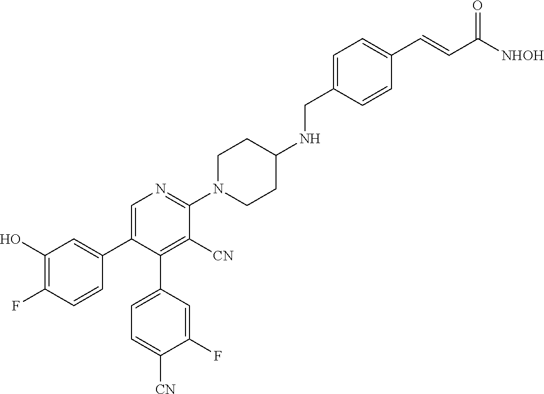 | ¹H NMR (400 MHz, Methanol-d4) δ 8.49 (s, 1H), 8.43 (s, 1H), 7.75 (dd, J = 8.0, 6.4 Hz, 1H), 7.62 (t, J = 9.2 Hz, 3H), 7.52 (d, J = 7.6 Hz, 2H), 7.39 (dd, J = 9.6, 1.6 Hz, 1H), 7.23 (dd, J = 8.0, 1.6 Hz, 1H), 6.95 (dd, J = 11.2, 8.4 Hz, 1H), 6.63 (dd, J = 8.4, 2.0 Hz, 1H), 6.57- 6.46 (m, 2H), 4.46 (d, J = 13.2 Hz, 2H), 4.20 (s, 2H), 3.32 (s, 1H), 3.17 (t, J = 12.8 Hz, 2H), 2.27 (d, J = 11.2 Hz, 2H), 1.87-1.71 (m, 2H). | 607.2 |
| 98 | (E)-3-(3-((1-(3- Cyano-4-(4- cyano-3- fluorophenyl)-5- (4-hydroxy-3- methoxyphenyl) pyridin-2- yl)piperidin-4- yl)amino)methyl) phenyl)-N- hydroxyacrylamide formate | 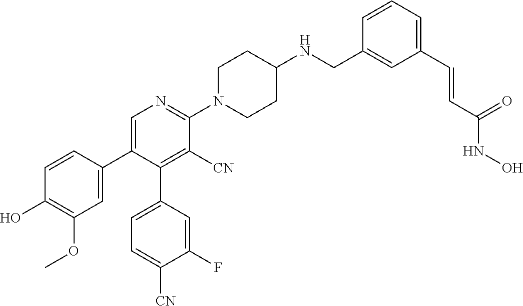 [SMILES: COc1cc(-c2cnc(N3CCC(NCc4cccc(/C=C/C(=O)NO)c4)CC3)c(C#N)c2-c2ccc(C#N)c(F)c2)ccc1O] | ¹H NMR (400 MHz, Methanol- d4) δ ppm 8.52 (s, 1H), 8.45 (s, 1H), 8.31 (s, 1H), 7.92 (t, J = 7.4 Hz, 1H), 7.64-7.54 (m, 2H), 7.45 (d, J = 5.6 Hz, 1H), 7.43-7.36 (m, 2H), 7.29 (dd, J = 8.0, 1.6 Hz, 1H), 6.65 (d, J = 8.2 Hz, 1H), 6.57- 6.43 (m, 3H), 4.18 (d, J = 12.8 Hz, 2H), 3.84 (s, 2H), 3.53 (s, 3H), 3.13 (d, J = 24.4 Hz, 2H), 2.80 (s, 1H), 2.02 (d, J = 12.4 Hz, 2H), 1.47 (q, J = 11.0 Hz, 2H). | 619.2 |
| 99 | (E)-3-(4-(((1-(3- Cyano-4-(4- cyano-3- fluorophenyl)-5- (4-hydroxy-3- methoxyphenyl) pyridin-2- yl)piperidin-4- yl)(methyl)amino) methyl)phenyl)-N- hydroxyacrylamide formate | 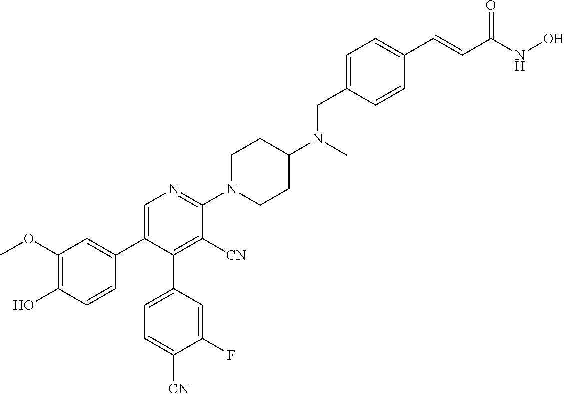 [SMILES: COc1cc(-c2cnc(N3CCC(N(C)Cc4ccc(/C=C/C(=O)NO)cc4)CC3)c(C#N)c2-c2ccc(C#N)c(F)c2)ccc1O] | ¹H NMR (400 MHz, DMSO-d6) δ 10.73 (s, 1H), 9.05 (s, 1H), 8.42 (s, 1H), 7.95 (dd, J = 8.0, 6.8 Hz, 1H), 7.66 (dd, J = 10.2, 1.6 Hz, 1H), 7.51 (d, J = 7.8 Hz, 2H), 7.44 (d, J = 15.8 Hz, 1H), 7.36 (d, J = 7.8 Hz, 2H), 7.28 (dd, J = 8.0, 1.6 Hz, 1H), 6.80 (d, J = 8.0 Hz, 1H), 6.52- 6.39 (m, 3H), 4.34 (d, J = 12.8 Hz, 2H), 3.56 (s, 3H), 3.61 (s, 2H), 3.07 (t, J = 12.4 Hz, 2H), 2.80- 2.62 (m, 1H), 2.14 (s, 3H), 1.93 (d, J = 12.2 Hz, 2H), 1.77-1.54 (m, 2H). | 633.3 |
| 100 | (E)-3-(4-((1-(3- Cyano-4-(4- cyano-3- fluorophenyl)-5- (4-hydroxy-3- methoxyphenyl) pyridin-2- yl)piperidin-4- yl)amino)methyl) phenyl)-N- hydroxyacrylamide hydrochloride | 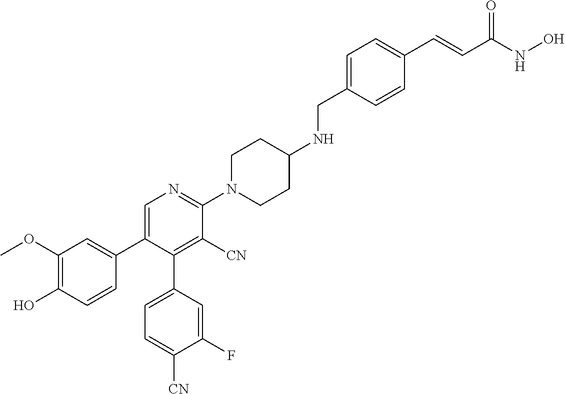 [SMILES: COc1cc(-c2cnc(N3CCC(NCc4ccc(/C=C/C(=O)NO)cc4)CC3)c(C#N)c2-c2ccc(C#N)c(F)c2)ccc1O] | ¹H NMR (400 MHz, DMSO-d6) δ 10.79 (s, 1H), 9.04 (d, J = 20.6 Hz, 2H), 8.90 (s, 2H), 8.47 (s, 1H), 7.97 (t, J = 7.4 Hz, 1H), 7.71-7.62 (m, 3H), 7.56 (d, J = 7.8 Hz, 2H), 7.48 (d, J = 15.6 Hz, 1H), 7.28 (d, J = 8.0 Hz, 1H), 6.82 (d, J = 8.0 Hz, 1H), 6.55-6.44 (m, 3H), 4.41- 4.19 (m, 4H), 3.53 (s, 3H), 3.43 (s, 1H), 3.13 (t, J = 12.6 Hz, 2H), 2.25 (d, J = 12.6 Hz, 2H), 1.82-1.67 (m, 2H). | 619.2 |
| 101 | (E)-3-(4-((1-(5-(4- Chloro-3- hydroxyphenyl)-3- cyano-4-(4-cyano- 3- fluorophenyl) pyridin- 2-yl)piperidin- 4- yl)amino)methyl) phenyl)-N- hydroxyacrylamide formate | 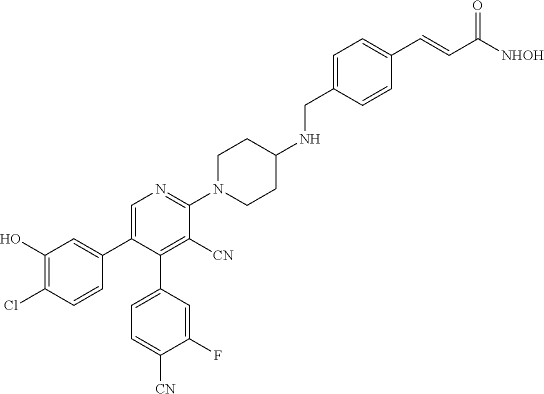 | ¹H NMR (400 MHz,Methanol-d4) δ 8.49 (s, 1H), 8.44 (s, 1H), 7.75 (t, J = 7.2 Hz, 1H), 7.62 (t, J = 8.4 Hz, 2H), 7.52 (d, J = 8.0 Hz, 3H), 7.41 (dd, J = 9.6, 1.2 Hz, 1H), 7.26- 7.14 (m, 2H), 6.62 (d, J = 2.0 Hz, 1H), 6.57-6.46 (m, 2H), 4.47 (d, J = 13.2 Hz, 2H), 4.19 (s, 2H), 3.30 (s, 1H), 3.18 (t, J = 12.4 Hz, 2H), 2.27 (d, J = 12.0 Hz, 2H), 1.79 (dd, J = 12.4, 3.9 Hz, 2H). | 623.2 |
| 102 | (E)-3-(4-(((1-(3- Cyano-4-(4- cyano-3- fluorophenyl)-5- (3-hydroxy-4- methylphenyl) pyridin- 2-yl)piperidin- 4- yl)amino)methyl) phenyl)-N- hydroxyacrylamide formate | 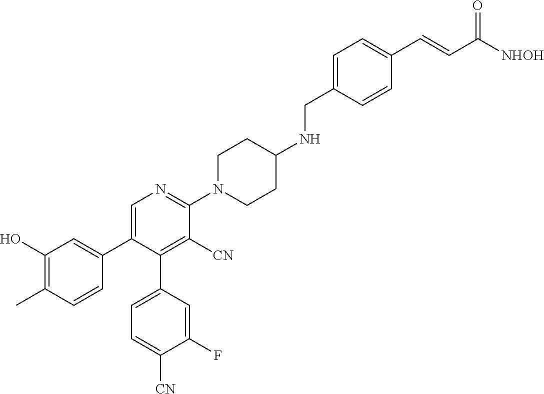 | ¹H NMR (400 MHz, Methanol- d4) δ 8.52 (s, 1H),8.39 (s, 1H), 7.69 (dd, J = 8.0, 6.6 Hz, 1H), 7.60 (d, J = 8.0 Hz, 2H), 7.56 (s, 1H), 7.48 (d, J = 7.8 Hz, 2H), 7.32 (dd, J = 9.8, 1.6 Hz, 1H), 7.17 (dd, J = 8.0, 1.5 Hz, 1H), 6.92 (d, J = 8.0 Hz, 1H), 6.50-6.37 (m, 3H), 4.40 (d, J = 13.6 Hz, 2H), 4.18 (s, 2H), 3.12 (t, J = 13.2 Hz, 3H), 2.24 (d, J = 12.2 Hz, 2H), 2.08 (s, 3H), 1.83- 1.70 (m, 2H). | 603.2 |
| 103 | (E)-3-(4-(((1-(5- (Benzo[d][1,3] dioxol-5- yl)-3-cyano- 4-(4-cyano-3- fluorophenyl) pyridin- 2-yl)piperidin- 4- yl)amino)methyl) phenyl)-N- hydroxyacrylamide formate | 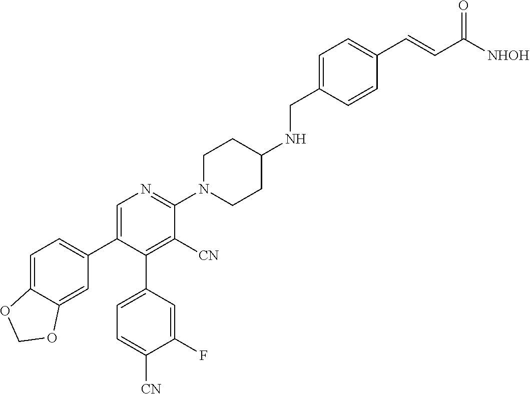 | ¹H NMR (400 MHz, Methanol- d4) δ 8.51(d, J = 2.7 Hz, 1H), 8.43 (d, J = 2.8 Hz, 1H), 8.38 (s, 1H), 7.71 (t, J = 7.2 Hz, 1H), 7.59 (d, J = 7.8 Hz, 2H), 7.54 (d, J = 15.8 Hz, 1H), 7.47 (d, J = 7.8 Hz, 2H), 7.36- 7.29 (m, 1H), 7.21 (d, J = 8.2 Hz, 1H), 6.69 (d, J = 8.6 Hz, 1H), 6.54- 6.50 (m, 2H), 6.47 (d, J = 15.8 Hz, 1H), 5.88 (s, 2H), 4.40 (d, J = 13.4 Hz, 2H), 4.13 (s, 2H), 3.44 (s, 1H), 3.12 (t, J = 12.8 Hz, 2H), 2.22 (d, J = 12.4 Hz, 2H), 1.73 (q, J = 11.6 Hz, 2H). | 617.2 |
| 104 | (E)-3-(6-((1-(3- Cyano-4-(4- cyano-3- fluorophenyl)-5- (3-hydroxy-4- methoxyphenyl) pyridin-2- yl)piperidin-4- yl)amino)methyl) pyridin-3-yl)-N- hydroxyacrylamide formate | 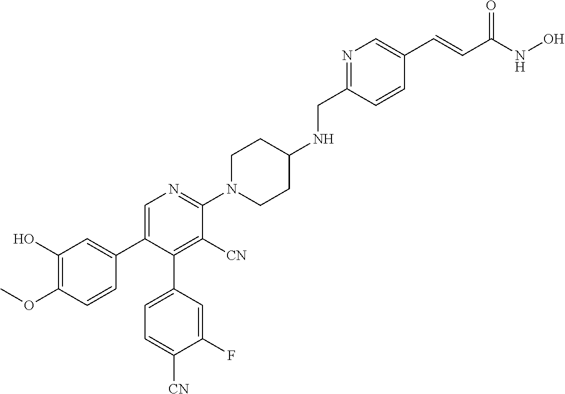 [SMILES: COc1ccc(-c2cnc(N3CCC(NCc4ccc(/C=C/C(=O)NO)cn4)CC3)c(C#N)c2-c2ccc(C#N)c(F)c2)cc1O] | ¹H NMR (400 MHz, DMSO-d6) δ10.82 (s, 1H), 9.02 (s, 1H),8.67 (d, J = 2.0 Hz, 1H), 8.41 (s, 1H), 8.26 (s, 1H), 7.94 (t, J = 7.6 Hz, 2H), 7.65 (d, J = 10.0 Hz, 1H), 7.57- 7.45 (m, 2H), 7.27 (dd, J = 8.0, 1.6 Hz, 1H), 6.80 (d, J = 8.0 Hz, 1H), 6.54 (d, J = 16.0 Hz, 1H), 6.47 (d, J = 7.2 Hz, 2H), 4.16 (dd, J = 10.8, 6.8 Hz, 2H), 3.91 (s, 2H), 3.71 (s, 3H), 3.18 (t, J = 12.0 Hz, 2H), 2.74 (t, J = 4.4 Hz, 1H), 1.98 (dd, J = 13.2, 4.0 Hz, 2H), 1.45 (d, J = 11.2 Hz, 2H). | 620.2 |
| 105 | (E)-3-(5-(((1-(3- Cyano-4-(4- cyano-3- fluorophenyl)-5- (3-hydroxy-4- methoxyphenyl) pyridin-2- yl)piperidin-4- yl)amino)methyl) thiophen-2-yl)-N- hydroxyacrylamide formate | 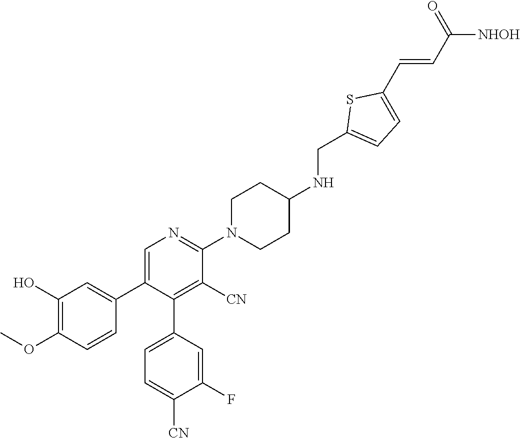 | ¹H NMR (400 MHz, DMSO-d6) δ 10.62 (s, 1H), 8.98 (s, 2H), 8.41 (s, 1H), 8.21 (s, 1H), 7.95 (t, J = 7.6 Hz, 1H), 7.59 (m, 2H), 6.94 (d, J = 3.6 Hz, 1H), 6.80 (d, J = 8.0 Hz, 1H), 6.47 (d, J = 8.0 Hz, 2H), 6.10 (d, J = 16.4 Hz, 1H), 4.17 (d, J = 13.2 Hz, 2H), 3.95 (s, 2H), 3.71 (s, 3H), 3.19 (d, J = 12.4 Hz, 2H), 2.80- 2.71 (m, 1H), 1.97 (d, J = 12.4 Hz, 2H), 1.43 (m, 2H). | 625.2 |
| 106 | (E)-3-(5-(((1-(3- Cyano-4-(4- cyano-3- fluorophenyl)-5- (3-hydroxy-4- methoxyphenyl) pyridin-2- yl)piperidin-4- yl)amino)methyl) pyrimidin-2-yl)-N- hydroxyacrylamide formate | 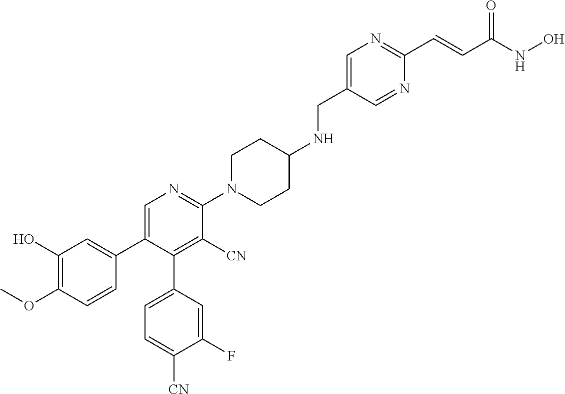 [SMILES: COc1ccc(-c2cnc(N3CCC(NCc4cnc(/C=C/C(=O)NO)nc4)CC3)c(C#N)c2-c2ccc(C#N)c(F)c2)cc1O] | ¹H NMR (400 MHz, DMSO-d6) δ 11.03 (s, 1H), 9.22 (s, 1H), 8.99 (s, 1H), 8.82 (s, 2H), 8.41 (s, 1H), 8.14 (s, 1H), 7.95 (t, J = 7.6 Hz, 1H), 7.66 (dd, J = 10.0, 1.6 Hz, 1H), 7.40-7.24 (m, 2H), 7.14 (d, J = 15.6 Hz, 1H), 6.80 (d, J = 8.0 Hz, 1H), 6.58-6.38 (m, 2H), 4.18 (d, J = 13.2 Hz, 2H), 3.84 (s, 2H), 3.71 (s, 3H), 3.23-3.14 (m, 2H), 2.74 (s, 1H), 2.08-1.94 (m, 2H), 1.45 (m, 2H). | 621.2 |
| 107 | 4-(3-(3-Cyano-4- (4-cyano-3- fluorophenyl)-5- (3-hydroxy-4- methoxyphenyl) pyridin-2- yl)piperidin-4- yl)amino)propyl)- N- hydroxybenzamide formate | 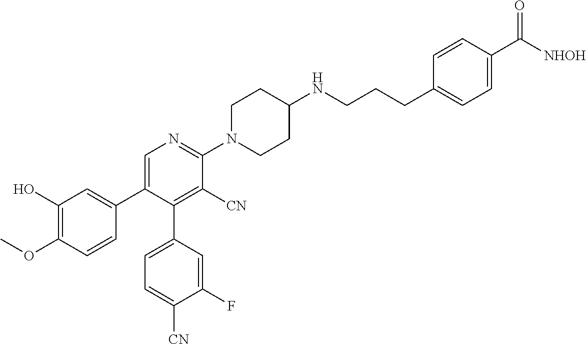 | ¹H NMR (400 MHz, DMSO-d6) δ 11.11 (s, 1H), 9.00 (s, 1H), 8.42 (s, 1H), 8.24 (s, 1H), 7.95 (t, J = 7.6 Hz, 1H), 7.72-7.56 (m, 3H), 7.28 (dd, J = 8.4, 6.4 Hz, 3H), 6.80 (d, J = 8.0 Hz, 1H), 6.47 (d, J = 8.0 Hz, 2H), 4.19 (d, J = 13.2 Hz, 2H), 3.71 (s, 3H), 3.16 (t, J = 12.0 Hz, 2H), 2.82 (s, 1H), 2.67 (q, J = 8.4, 8.0 Hz, 4H), 1.97 (d, J = 12.4 Hz, 2H), 1.76 (p, J = 7.2 Hz, 2H), 1.51- 1.34 (m, 2H). | 621.3 |
| 108 | (E)-3-(4-(((1-(3- Cyano-4-(4- cyano-3- fluorophenyl)-5- (3-hydroxy-4- methoxyphenyl) pyridin-2- yl)piperidin-4- yl)amino)methyl)- 2- methoxyphenyl)- N- hydroxyacrylamide formate | 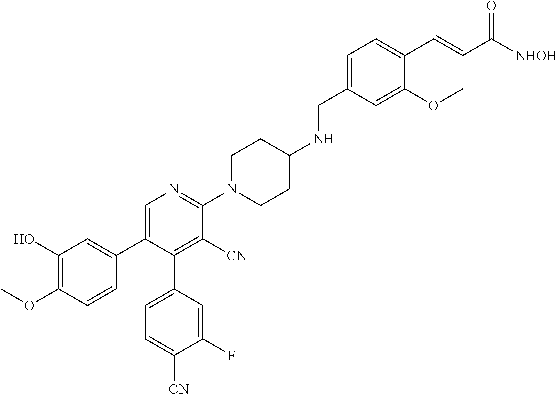 | ¹H NMR (400 MHz, DMSO-d6) δ 10.69 (s, 1H), 8.90 (s, 1H), 8.41 (s, 1H), 8.19 (s, 1H), 7.95 (t, J = 7.6 Hz, 1H), 7.74-7.60 (m, 2H), 7.46 (d, J = 7.6 Hz, 1H), 7.27 (dd, J = 8.0, 1.6 Hz, 1H), 7.12 (s, 1H), 7.00 (d, J = 7.6 Hz, 1H), 6.80 (d, J = 8.0 Hz, 1H), 6.57-6.39 (m, 3H), 4.18 (dd, J = 10.4, 7.2 Hz, 2H), 3.87 (s, 3H), 3.84 (s, 2H), 3.71 (s, 3H), 3.25-3.11 (m, 2H), 2.77 (m, 1H), 2.10-1.95 (m, 2H), 1.48 (m, 2H). | 649.3 |
| 109 | 3-(4-(1-(3-Cyano- 4-(4-cyano-3- fluorophenyl)-5- (3-hydroxy-4- methoxyphenyl) pyridin-2- yl)piperidin-4- yl)amino)methyl) phenyl)-N- hydroxypropiolamide formate | 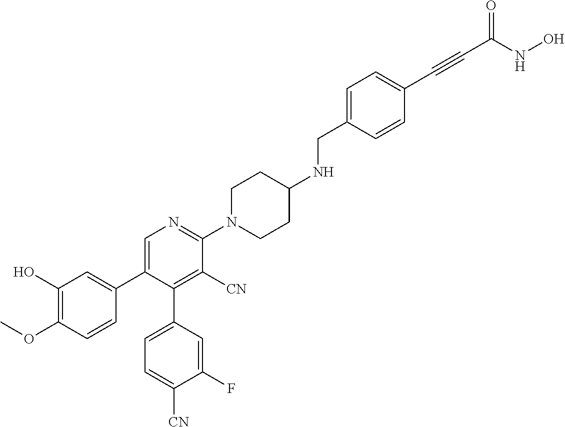 [SMILES: COc1ccc(-c2cnc(N3CCC(NCc4ccc(C#CC(=O)NO)cc4)CC3)c(C#N)c2-c2ccc(C#N)c(F)c2)cc1O] | ¹H NMR (400 MHz, DMSO-d6) δ ppm: 11.30 (s, 1H), 9.35 (s, 1H), 9.01 (s, 1H), 8.44 (s, 1H), 7.97 (t, J = 7.4 Hz, 1H), 7.67 (dd, J = 10.2, 1.4 Hz, 1H), 7.61 (s, 3H), 7.28 (dd, J = 8.0, 1.4 Hz, 1H), 6.81 (d, J = 8.0 Hz, 1H), 6.46 (s, 2H), 4.27 (d, J = 13.0 Hz, 2H), 4.14 (s, 1H), 3.72 (s, 3H), 3.31 (s, 2H),3.14 (t, J = 12.6 Hz, 2H), 2.17 (s, 2H), 1.67 (s, 2H). | 617.2 |
| 110 | (E)-3-(4-((1-(3- Cyano-4-(4- cyano-3- fluorophenyl)-5- (5-fluoro-3- methylbenzo[d] isoxazol-6- yl)pyridin-2- yl)piperidin-4- yl)amino)methyl) phenyl)-N- hydroxyacrylamide formate | 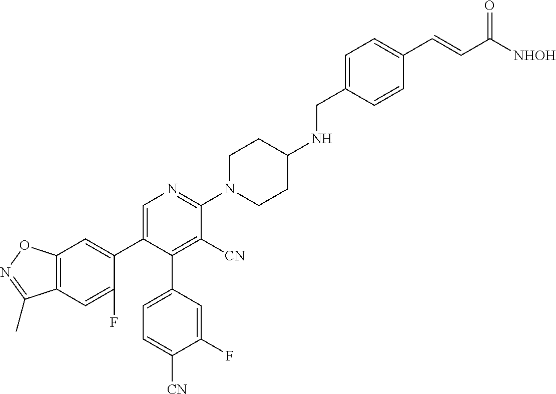 | ¹H NMR (400 MHz, DMSO-d6) δ ppm: 10.73 (s, 1H), 9.02 (s, 1H), 8.52 (s, 1H), 8.16 (s, 1H), 7.91 (t, J = 7.4 Hz, 1H), 7.75 (dd, J = 16.2, 7.8 Hz, 1H), 7.61 (d, J = 8.8 Hz, 1H), 7.53 (d, J = 7.8 Hz, 2H), 7.49- 7.40 (m, 3H), 7.31 (d, J = 8.0 Hz, 1H), 6.44 (d, J = 15.8 Hz, 1H), 4.30 (d, J = 13.4 Hz, 2H), 3.86 (s, 2H), 3.22 (s, 2H), 2.87-2.78 (m, 1H), 2.48 (s, 3H), 2.07-1.99 (m, 2H), 1.49 (q, J = 11.9, 10.7 Hz, 2H). | 646.2 |
| 111 | (E)-3-(4-((1-(3- Cyano-4-(4- cyano-3- fluorophenyl)-5- (4-cyclopropyl-3- hydroxyphenyl) pyridin-2- yl)piperidin-4- yl)amino)methyl) phenyl)-N- hydroxyacrylamide formate | 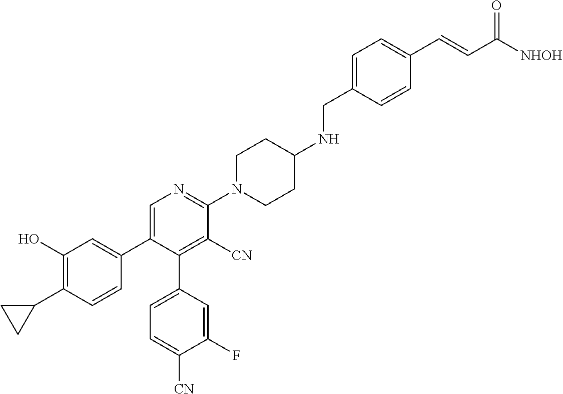 | 1H NMR (400 MHz, DMSO-d6) δ 10.72 (s, 1H), 9.32 (s, 1H), 9.01 (s, 1H), 8.40 (s, 1H), 7.94 (t, J = 7.4 Hz, 1H), 7.68 (dd, J = 10.0, 1.4 Hz, 1H), 7.64-7.29 (m, 5H), 7.24 (dd, J = 8.0, 1.4 Hz, 1H), 6.63 (d, J = 8.0 Hz, 1H), 6.52-6.31 (m, 3H), 4.18 (d, J = 13.0 Hz, 2H), 3.83 (s, 2H), 3.17 (t, J = 5.8 Hz, 2H), 2.76 (s, 1H), 1.99 (ddd, J = 10.6, 8.2, 4.8 Hz, 3H), 1.46 (d, J = 11.0 Hz, 2H), 0.94-0.75 (m, 2H), 0.64-0.50 (m, 2H). | 629.3 |
| 112 | (E)-3-(5-((1-(3- Cyano-4-(4- cyano-3- fluorophenyl)-5- (3-hydroxy-4- methoxyphenyl) pyridin-2- yl)piperidin-4- yl)amino)methyl) thiazol-2-yl)-N- hydroxyacrylamide formate | 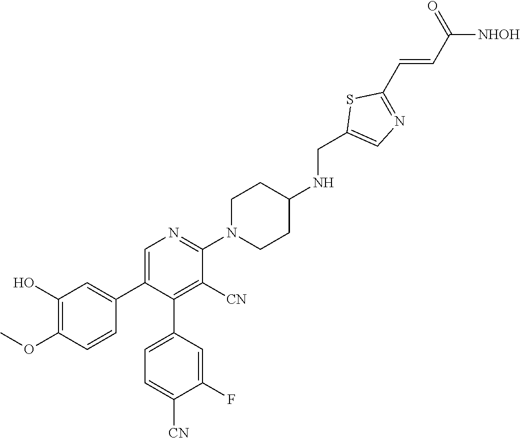 | ¹H NMR (400 MHz, Methanol-d4) δ 8.40 (s, 1H), 8.39 (s, 1H), 7.84 (s, 1H), 7.73 (t, J = 7.2 Hz, 1H), 7.62 (d, J = 15.6 Hz, 1H), 7.36 (dd, J = 9.6, 1.6 Hz, 1H), 7.23 (dd, J = 8.0, 1.6 Hz, 1H), 6.85-6.78 (m, 1H), 6.73 (d, J = 15.6 Hz, 1H), 6.50 (d, J = 6.8 Hz, 2H), 4.36 (d, J = 13.2 Hz, 2H), 4.26 (s, 2H), 3.81 (s, 3H), 3.17 (t, J = 12.0 Hz, 2H), 3.09- 2.97 (m, 1H), 2.19-2.09 (m, 2H), 1.65 (qd, J = 12.0, 4.0 Hz, 2H). | 626.2 |
| 113 | (E)-3-(4-(((1-(3- Cyano-4-(4- cyano-3- fluorophenyl)-5- (4-cyano-3- hydroxyphenyl) pyridin-2- yl)piperidin-4- yl)amino)methyl) phenyl)-N- hydroxyacrylamide formate | 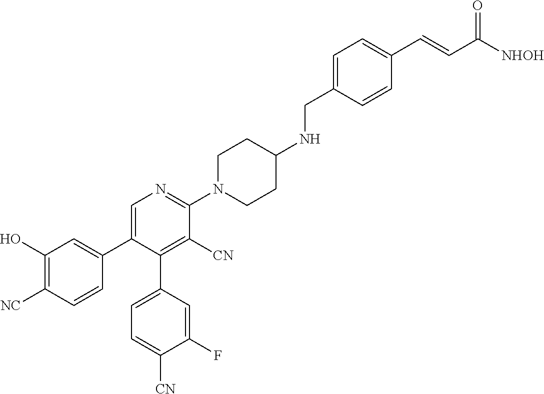 | ¹H NMR (400 MHz, DMSO-d6) δ 10.72 (s, 1H), 9.01 (s, 1H), 8.50- 8.43 (m, 1H), 8.14 (s, 1H), 7.96 (t, J = 7.4 Hz, 1H), 7.74 (d, J = 10.0 Hz, 1H), 7.51 (s, 3H), 7.46 (d, J = 26.6 Hz, 2H), 7.26 (dd, J = 8.0, 1.4 Hz, 1H), 6.67 (d, J = 7.8 Hz, 1H), 6.63 (d, J = 1.6 Hz, 1H), 6.44 (d, J = 15.9 Hz, 1H), 4.25 (d, J = 12.9 Hz, 2H), 3.84 (d, J = 10.9 Hz, 2H), 3.20 (d, J = 12.1 Hz, 2H), 2.79 (s, 1H), 2.01 (s, 2H), 1.47 (s, 2H). | 614.2 |
| 114 | (E)-3-(3-(3- Cyano-4-(4- cyano-3- fluorophenyl)-5- (3-hydroxy-4- methoxyphenyl) pyridin-2- yl)piperidin-4- yl)amino)methyl) phenyl)-N- hydroxyacrylamide formate | 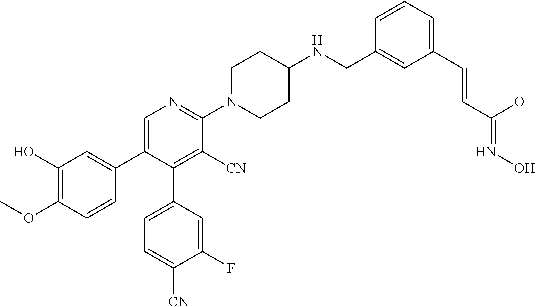 [SMILES: COc1ccc(-c2cnc(N3CCC(NCc4cccc(/C=C/C(O)=N/O)c4)CC3)c(C#N)c2-c2ccc(C#N)c(F)c2)cc1O] | ¹H NMR (400 MHz, DMSO-d6) δ ppm 10.80 (s, 1H), 9.01 (s, 2H), 8.43 (s, 1H), 7.96 (t, J = 7.4 Hz, 1H), 7.77-7.63 (m, 2H), 7.55- 7.37 (m, 4H), 7.28 (dd, J = 8.0, 1.6 Hz, 1H), 6.81 (d, J = 8.0 Hz, 1H), 6.55-6.42 (m, 3H), 4.23 (d, J = 13.0 Hz, 2H), 3.99 (s, 2H), 3.71 (s, 3H), 3.16 (t, J = 12.2 Hz, 2H), 3.01 (s, 1H), 2.11 (d, J = 12.2 Hz, 2H), 1.58 (d, J = 11.8 Hz, 2H). | 619.2 |
| 115 | 2-(2-(1-(3-Cyano- 4-(4-cyano-3- fluorophenyl)-5- (3-hydroxy-4- methoxyphenyl) pyridin-2- yl)piperidin-4- yl)amino)ethyl) amino)-N- hydroxypyrimidine- 5-carboxamide formate | 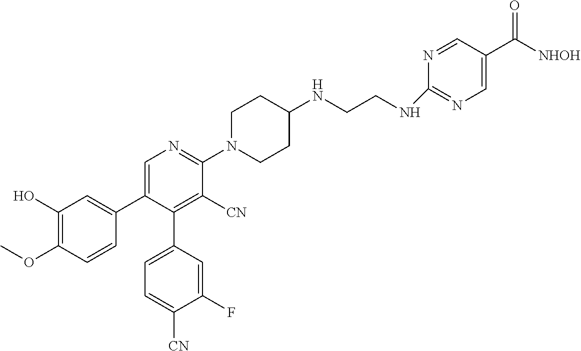 | ¹H NMR (400 MHz, Methanol-d4) δppm 8.71 (s, 2H), 8.53 (s, 1H), 8.45 (s, 1H), 7.76 (t, J = 7.4 Hz, 1H), 7.38 (dd, J = 9.8, 1.3 Hz, 1H), 7.25 (dd, J = 7.8, 1.4 Hz, 1H), 6.84 (d, J = 8.6 Hz, 1H), 6.53 (d, J = 6.6 Hz, 2H), 4.44 (d, J = 13.4 Hz, 2H), 3.83 (s, 3H), 3.77 (t, J = 5.9 Hz, 2H), 3.38 (s, 1H), 3.31-3.25 (m, 2H), 3.18 (t, J = 12.6 Hz, 2H), 2.23 (d, J = 12.0 Hz, 2H), 1.93-1.65 (m, 2H). | 624.2 |
| 116 | (E)-3-(4-(1-(3- Cyano-4-(4- cyano-3- fluorophenyl)-5- (3-hydroxy-4- (trifluoromethoxy) phenyl)pyridin-2- yl)piperidin-4- yl)amino)methyl) phenyl)-N- hydroxyacrylamide formate | 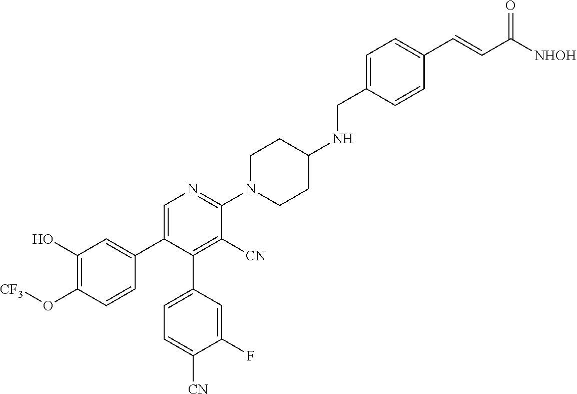 | ¹H NMR (400 MHz, Methanol-d4) δ ppm 8.53 (s,1H), 8.48 (d, J = 13.8 Hz, 2H), 7.76 (dd, J = 7.8, 6.8 Hz, 1H), 7.62 (t, J = 6.8 Hz, 2H), 7.51 (d, J = 7.8 Hz, 2H), 7.41 (dd, J = 9.8, 1.4 Hz, 1H), 7.24 (dd, J = 8.0, 1.6 Hz, 1H), 7.11 (dd, J = 8.6, 1.6 Hz, 1H), 6.68 (d, J = 2.2 Hz, 1H), 6.59 (dd, J = 8.2, 2.0 Hz, 1H), 6.51 (d, J = 15.8 Hz, 1H), 4.48 (d, J = 13.2 Hz, 2H), 4.17 (s, 2H), 3.27 (s, 1H), 3.18 (t, J = 12.6 Hz, 2H), 2.33- 2.20 (m, 2H), 1.86-1.68 (m, 2H). | 673.2 |
| 117 | (E)-3-(5-((1-(3- Cyano-4-(4- cyano-3- fluorophenyl)-5- (3-hydroxy-4- methoxyphenyl) pyridin-2- yl)piperidin-4- yl)amino)methyl) pyridin-2-yl)-N- hydroxyacrylamide formate | 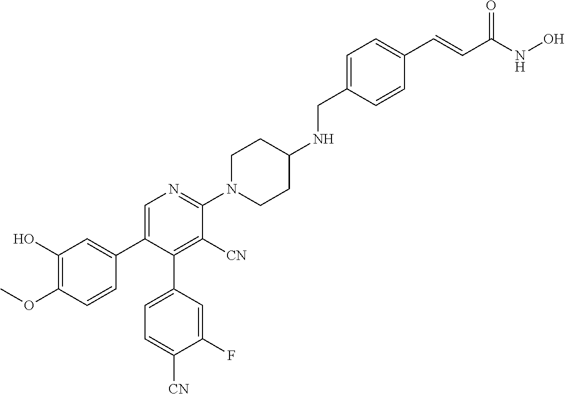 [SMILES: COc1ccc(-c2cnc(N3CCC(NCc4ccc(/C=C/C(=O)NO)cc4)CC3)c(C#N)c2-c2ccc(C#N)c(F)c2)cc1O] | ¹H NMR (400 MHz, Methanol-d4) δppm 8.69 (s, 1H), 8.46 (d, J = 11.6 Hz, 2H), 8.01-7.92 (m, 1H), 7.76 (dd, J = 7.8, 6.6 Hz, 1H), 7.69- 7.58 (m, 2H), 7.39 (dd, J = 9.6, 1.4 Hz, 1H), 7.26 (dd, J = 8.0, 1.4 Hz, 1H), 6.94 (d, J = 15.4 Hz, 1H), 6.88- 6.75 (m, 1H), 6.62-6.42 (m, 2H), 4.43 (d, J = 13.2 Hz, 2H), 4.17 (s, 2H), 3.84 (s, 3H), 3.20 (t, J = 12.6 Hz, 3H), 2.34-2.11 (m, 2H), 1.85-1.67 (m, 2H). | 620.2 |
| 118 | (E)-3-(4-((1-(3- Cyano-4-(4- cyanophenyl)-5- (3-hydroxy-4- methoxyphenyl) pyridin-2- yl)piperidin-4- yl)amino)methyl) phenyl)-N- hydroxyacrylamide formate | 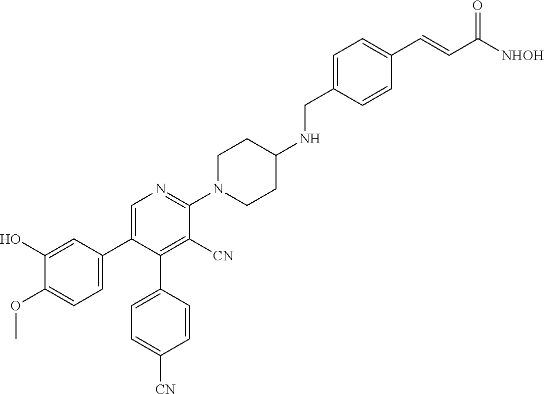 | ¹H NMR (400 MHz, DMSO-d6) δ ppm: 10.73 (s, 1H), 8.96 (s, 1H), 8.39 (s, 1H), 8.17 (s, 1H), 7.86 (d, J = 8.0 Hz, 2H), 7.53 (td, J = 10.4, 9.0, 7.8 Hz, 3H),7.48 (td, J = 19.8, 19.0, 7.8 Hz, 4H), 6.77 (d, J = 8.2 Hz, 1H), 6.49-6.39 (m, 3H), 4.22- 4.12 (m, 2H), 3.86 (s, 2H), 3.70 (s, 3H), 3.14 (s, 2H), 2.80 (tt, J = 9.7, 4.0 Hz, 1H), 2.01 (dd, J = 13.1, 3.9 Hz, 2H), 1.56-1.41 (m, 2H). | 601.2 |
| 119 | (E)-3-(4-((2-(3- Cyano-4-(4- cyano-3- fluorophenyl)-5- (3-hydroxy-4- methoxyphenyl) pyridin-2-yl)-2- azabicyclo[2.2.1] heptan-5- yl)amino)methyl) phenyl)-N- hydroxyacrylamide formate | 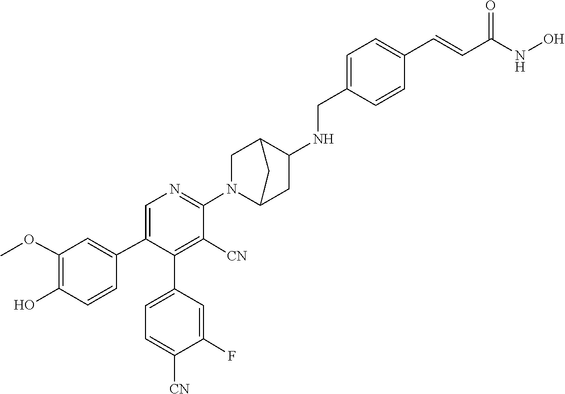 [SMILES: COc1cc(-c2cnc(N3CC4CC3CC4NCc3ccc(/C=C/C(=O)NO)cc3)c(C#N)c2-c2ccc(C#N)c(F)c2)ccc1O] | ¹H NMR (400 MHz, DMSO-d₆) δ 10.72 (s, 1H), 8.98 (m, 2H), 8.30 (m, 1H), 7.94 (s, 1H), 7.67-7.27 (m, 6H), 6.77 (d, J = 8.6 Hz, 1H), 6.50-6.31 (m, 3H), 4.79 (m, 1H), 4.00-3.60 (m, 6H), 3.17 (t, J = 9.8 Hz, 1H), 2.78 (m, 2H), 2.20-1.81 (m, 2H), 1.71 (m, 1H), 1.38 (m, 1H). | 631.2 |
| 120 | 2-(4-(2-((1-(3- Cyano-4-(4- cyano-3- fluorophenyl)-5- (3-hydroxy-4- methoxyphenyl) pyridin-2- yl)piperidin-4- yl)amino)ethyl) phenyl)-N- hydroxyacetamide formate | 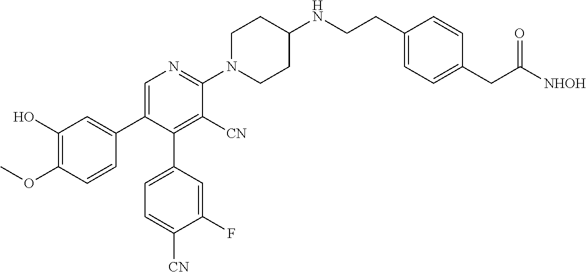 | ¹H NMR (400 MHz, DMSO-d6) δ ppm: 8.51 (s, 1H), 8.43 (s, 1H), 7.78-7.69 (m, 1H), 7.36 (dd, J = 9.8, 1.4 Hz, 1H), 7.33-7.19 (m, 5H), 6.85-6.78 (m, 1H), 6.56- 6.46 (m, 2H), 4.41 (d, J = 13.4 Hz, 2H), 3.81 (s, 3H), 3.39 (s, 2H), 3.25 (s, 2H), 3.15 (t, J = 12.6 Hz, 3H), 2.97 (s, 2H), 2.21 (d, J = 11.2 Hz, 2H), 1.75 (d, J = 11.0 Hz, 2H). | 621.2 |
| 121 | 4-(4-((1-(3-Cyano- 4-(4-cyano-3- fluorophenyl)-5- (3-hydroxy-4- methoxyphenyl) pyridin-2- yl)piperidin-4- yl)amino)methyl) piperidin-1-yl)-N- hydroxybenzamide formate | 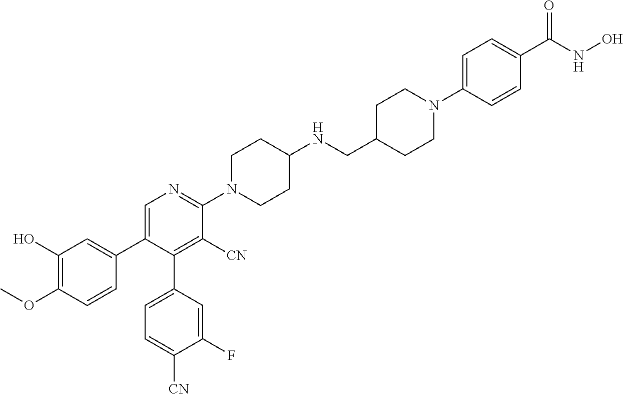 [SMILES: COc1ccc(-c2cnc(N3CCC(NCC4CCN(c5ccc(C(=O)NO)cc5)CC4)CC3)c(C#N)c2-c2ccc(C#N)c(F)c2)cc1O] | ¹H NMR (400 MHz, Methanol-d4) δ 8.42 (s, 1H), 7.83-7.68 (m, 1H), 7.64 (d, J = 8.6 Hz, 2H), 7.36 (dd, J = 9.7, 1.4 Hz, 1H), 7.23 (dd, J = 8.0, 1.4 Hz, 1H), 6.97 (d, J = 8.6 Hz, 2H), 6.82 (d, J = 8.6 Hz, 1H), 6.51 (d, J = 6.4 Hz, 2H), 4.41 (d, J = 8.2 Hz, 2H), 3.92 (d, J = 12.8 Hz, 2H), 3.81 (s, 3H), 3.16 (t, J = 12.8 Hz, 3H), 3.01-2.76 (m, 4H), 2.19 (d, J = 11.6 Hz, 2H), 1.90 (d, J = 13.0 Hz, 3H), 1.72 (d, J = 8.2 Hz, 2H), 1.41 (t, J = 5.4 Hz, 2H). | 676.3 |
| 123 | (E)-3-(4-((1-(5- Cyano-4-(4- cyano-3- fluorophenyl)-5′- hydroxy-6′- methoxy-[3,3′- bipyridin]-6- yl)piperidin-4- yl)amino)methyl) phenyl)-N- hydroxyacrylamide trifluoroacetate | 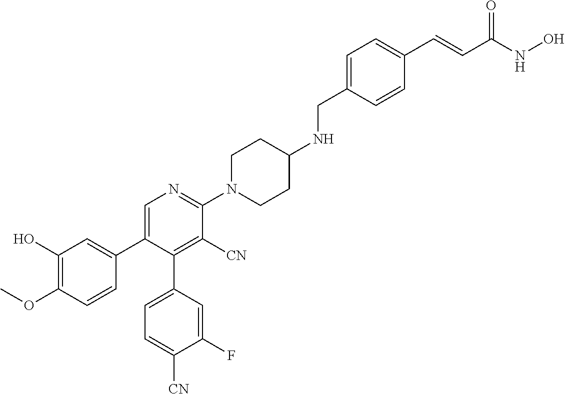 [SMILES: COc1ccc(-c2cnc(N3CCC(NCc4ccc(/C=C/C(=O)NO)cc4)CC3)c(C#N)c2-c2ccc(C#N)c(F)c2)cc1O] | ¹H NMR (400 MHz, Methanol-d4) δppm 8.46 (s, 1H), 7.79 (t, J = 7.4 Hz, 1H), 7.67 (d, J = 7.8 Hz, 2H), 7.66-7.52 (m, 3H), 7.43 (dd, J = 9.8, 1.4 Hz, 1H), 7.33 (d, J = 2.0 Hz, 1H), 7.25 (dd, J = 7.8, 1.4 Hz, 1H), 6.77 (d, J = 2.2 Hz, 1H), 6.53 (d, J = 15.8 Hz, 1H), 4.50 (d, J = 13.6 Hz, 2H), 4.33 (s, 2H), 3.92 (s, 3H), 3.51 (q, J = 9.4, 5.8 Hz, 1H), 3.19 (t, J = 12.6 Hz, 2H), 2.43- 2.24 (m, 2H), 1.88 (ddt, J = 22.0, 13.6, 7.2 Hz, 2H). | 620.2 |
| 124 | (E)-3-(4-(((1-(3- Cyano-4-(4- cyano-3- fluorophenyl)-5- (4- (difluoromethoxy)- 3- hydroxyphenyl) pyridin-2- yl))piperidin-4- yl)amino)methyl) phenyl)-N- hydroxyacrylamide formate | 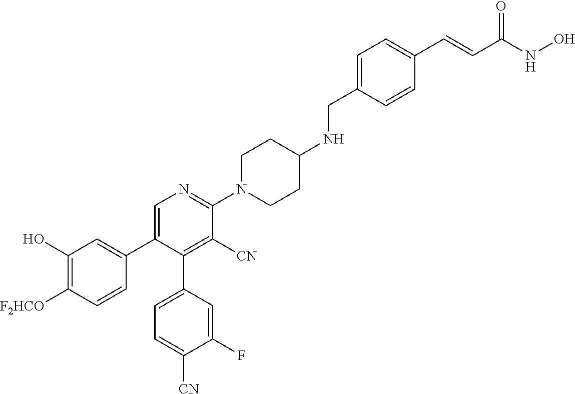 [SMILES: N#Cc1ccc(-c2c(-c3ccc(OC(F)F)c(O)c3)cnc(N3CCC(NCc4ccc(/C=C/C(=O)NO)cc4)CC3)c2C#N)cc1F] | ¹H NMR (400 MHz, DMSO-d6) δ 10.79 (s, 1H), 9.06 (s, 1H), 8.48 (s, 1H), 7.98 (t, J = 7.4 Hz, 1H), 7.71 (d, J = 10.0 Hz, 1H), 7.62 (d, J = 7.9 Hz, 2H), 7.52 (d, J = 8.0 Hz, 2H), 7.47 (d, J = 15.8 Hz, 1H), 7.29 (dd, J = 8.0, 1.4 Hz, 1H), 7.05- 6.95 (m, 2H), 6.62 (d, J = 2.1 Hz, 1H), 6.59-6.51 (m, 2H), 6.48 (s, 1H), 4.31 (d, J = 13.1 Hz, 2H), 4.15 (s, 2H), 3.33 (s, 1H), 3.16 (t, J = 12.5 Hz, 2H), 2.26-2.12 (m, 2H), 1.77-1.56 (m, 2H). | 655.2 |
| 125 | 2-(3-(3-Cyano-4- (4-cyano-3- fluorophenyl)-5- (3-hydroxy-4- methoxyphenyl) pyridin-2- yl)piperidin-4- yl)amino)propyl)- N- hydroxythiazole- 5-carboxamide formate | 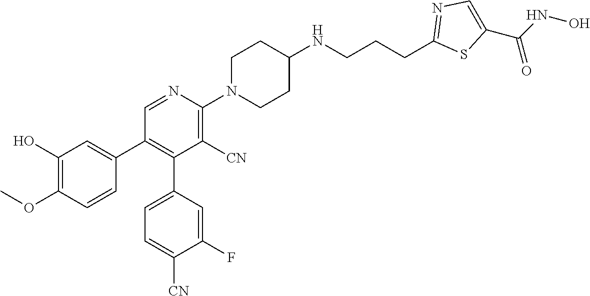 [SMILES: COc1ccc(-c2cnc(N3CCC(NCCCc4ncc(C(=O)NO)s4)CC3)c(C#N)c2-c2ccc(C#N)c(F)c2)cc1O] | ¹H NMR (400 MHz, DMSO-d6) δ 9.02 (s, 1H),8.42 (s, 1H), 8.23 (s, 1H), 8.09 (s, 1H), 7.95 (t, J = 7.4 Hz, 1H), 7.66 (dd, J = 10.0, 1.4 Hz, 1H), 7.27 (dd, J = 8.0, 1.4 Hz, 1H), 6.80 (d, J = 8.0 Hz, 1H), 6.46 (s, 2H), 4.19 (d, J = 13.0 Hz, 2H), 3.71 (s, 3H), 3.16 (t, J = 12.0 Hz, 2H), 3.06 (t, J = 7.4 Hz, 2H), 2.83 (s, 1H), 2.73 (t, J = 6.9 Hz, 2H), 2.06- 1.82 (m, 4H), 1.44 (q, J = 11.0 Hz, 2H). | 628.2 |
| 126 | 6-(1-(3-Cyano-4- (4-cyano-3- fluorophenyl)-5- (3-hydroxy-4- methoxyphenyl) pyridin-2- yl)piperidin-4- yl)amino)methyl)- N- hydroxybenzothio phene-2- carboxamide formate | 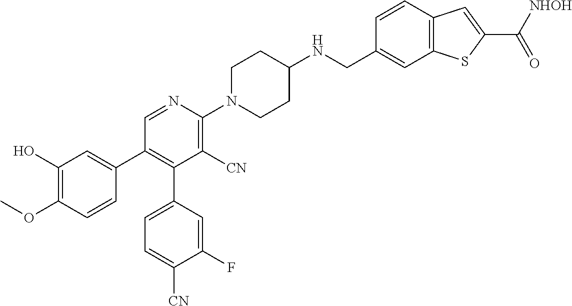 | ¹H NMR (400 MHz, Methanol-d4) δ 8.45 (s, 1H), 8.39 (s, 1H), 8.12 (s, 1H), 8.01 (d, J = 8.4 Hz, 1H), 7.88 (s, 1H), 7.75 (t, J = 7.2 Hz, 1H), 7.57 (d, J = 8.4 Hz, 1H), 7.37 (d, J = 9.6 Hz, 1H), 7.23 (dd, J= 8.0, 1.2 Hz, 1H), 6.89-6.77 (m, 1H), 6.51 (d, J = 6.4 Hz, 2H), 4.47 (d, J = 12.0 Hz, 4H), 4.03 (p, J = 7.6 Hz, 1H), 3.81 (s, 3H), 3.19 (t, J = 12.8 Hz, 2H), 2.36 (d, J = 12.4 Hz, 2H), 1.97-1.82 (m, 2H). | 649.2 |
| 127 | (E)-3-(4-(((1-(5- (4-Bromo-3- hydroxyphenyl)-3- cyano-4-(4-cyano- 3- fluorophenyl) pyridin- 2-yl)piperidin- 4- yl)amino)methyl) phenyl)-N- hydroxyacrylamide formate | 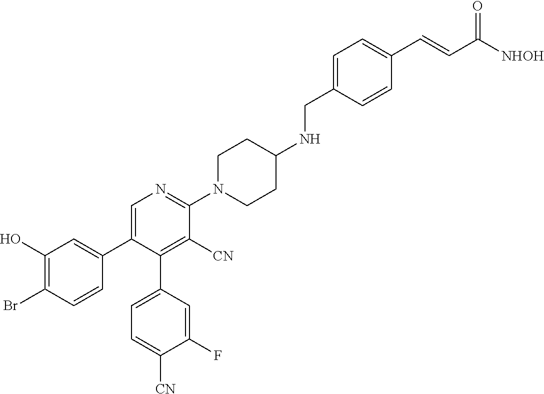 | ¹H NMR (400 MHz, DMSO-d6) δ ppm: 10.73 (s, 1H), 9.00 (s, 1H), 8.44 (s, 1H), 8.22-8.15 (m, 1H), 7.96 (t, J = 7.4 Hz, 1H), 7.53 (d, J = 7.8 Hz, 2H), 7.51-7.40 (m, 3H), 7.37 (d, J = 8.2 Hz, 1H), 7.26 (dd, J = 8.0, 1.4 Hz, 1H), 6.59 (d, J = 2.2 Hz, 1H), 6.51-6.40 (m, 2H), 4.54 (s, 2H), 4.23 (d, J = 12.8 Hz, 2H), 3.19 (t, J = 11.8 Hz, 2H), 2.83 (s, 1H), 2.03 (d, J = 12.4 Hz, 2H), 1.49 (q, J = 11.8, 11.54Hz, 2H). | 667.2 |
| 128 | 2-(4-((1-(3-Cyano- 4-(4-cyano-3- fluorophenyl)-5- (3-hydroxy-4- methoxyphenyl) pyridin-2- yl)piperidin-4- yl)amino)methyl)- 2- methoxyphenoxy)- N- hydroxyacetamide formate | 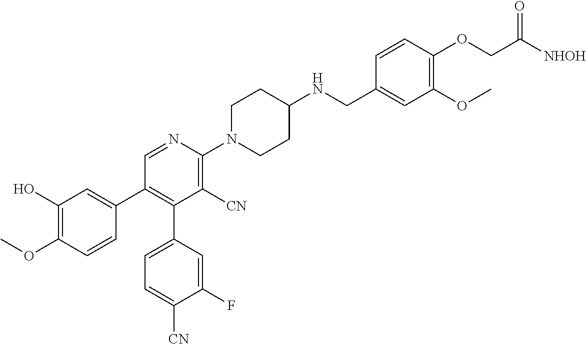 | ¹H NMR (400 MHz, DMSO-d6) δ 10.70 (s, 1H), 8.97 (s, 1H), 8.42 (s, 1H), 8.21 (s, 1H), 7.95 (t, J = 7.4 Hz, 1H), 7.66 (d, J = 10.0 Hz, 1H), 7.27 (dd, J = 8.0, 1.4 Hz, 1H), 7.06 (s, 1H), 6.95-6.69 (m, 3H), 6.47 (d, J = 8.0 Hz, 2H), 4.39 (s, 2H), 4.26-4.10 (m, 2H), 3.79 (d, J = 2.6 Hz, 5H), 3.71 (s, 3H), 3.16 (s, 2H), 2.88-2.76 (m, 1H), 2.02 (dd, J = 13.2, 4.0 Hz, 2H), 1.49 (q, J = 11.4, 9.8 Hz, 2H). | 653.2 |
| 129 | (E)-3-(4-(1-(3- Cyano-4-(4- cyano-3- fluorophenyl)-5- (3-hydroxy-4- (trifluoromethyl) phenyl)pyridin-2- yl)piperidin-4- yl)amino)methyl) phenyl)-N- hydroxyacrylamide formate | 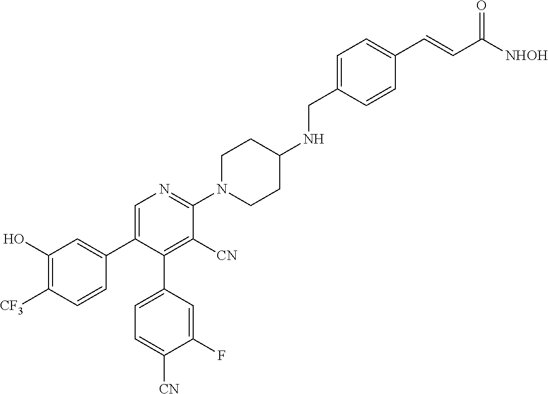 | ¹H NMR (400 MHz, Methanol-d4) δ ppm 8.52(s, 1H), 8.48 (d, J = 12.4 Hz, 1H), 7.76 (t, J = 7.2 Hz, 1H), 7.67-7.34 (m, 7H), 7.21 (dd, J = 8.0, 1.5 Hz, 1H), 6.65 (d, J = 8.6 Hz, 2H), 6.50 (d, J = 15.8 Hz, 1H), 4.50 (d, J = 13.4 Hz, 2H), 4.15 (s, 2H), 3.26 (s, 1H), 3.18 (t, J = 12.6 Hz, 2H), 2.35-2.21 (m, 2H), 1.83- 1.71 (m, 2H). | 657.2 |
| 130 | (E)-3-(4-((1-(3- Cyano-4-(4- cyano-3- fluorophenyl)-5- (2-fluoro-5- hydroxy-4- methoxyphenyl) pyridin-2- yl)piperidin-4- yl)amino)methyl) phenyl)-N- hydroxyacrylamide formate | 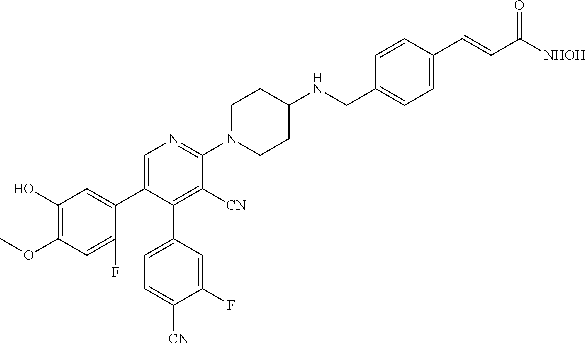 | ¹H NMR (400 MHz, Methanol- d4) δ 8.48 (s, 1H), 8.38 (s, 1H), 7.73 (t, J = 7.4 Hz, 1H), 7.67-7.41 (m, 5H), 7.37 (d, J = 9.7 Hz, 1H), 7.22 (d, J = 8.0 Hz, 1H), 6.61 (dd, J = 16.6, 9.2 Hz, 2H), 6.52 (d, J = 15.8 Hz, 1H), 4.49 (d, J = 13.6 Hz, 2H), 4.20 (s, 2H), 3.80 (s, 3H), 3.18 (t, J = 12.8 Hz, 3H), 2.28 (d, J = 12.2 Hz, 2H), 1.80 (d, J = 12.0 Hz, 2H). | 637.2 |
| 131 | (E)-3-(4-(((1-(3- Cyano-4-(4- cyano-3- fluorophenyl)-5- (3-hydroxy-4- (hydroxymethyl) phenyl)pyridin-2- yl)piperidin-4- yl)amino)methyl) phenyl)-N- hydroxyacrylamide formate | 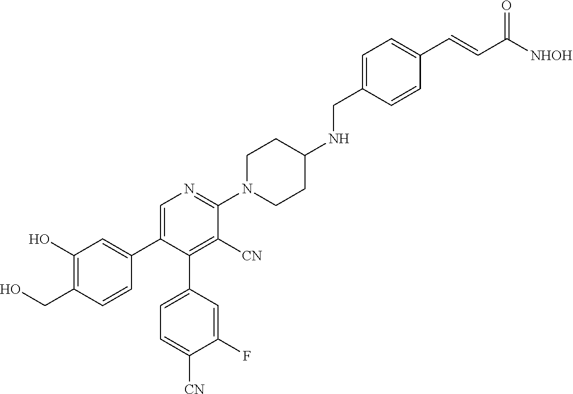 | ¹H NMR (400 MHz, Methanol- d4)δ ppm: 8.44(s, 1H),8.28(s, 1H), 7.73 (dd, J = 7.9, 6.7 Hz, 1H), 7.65 (d, J = 7.9 Hz, 2H), 7.64- 7.43 (m, 1H), 7.37 (dd, J = 9.7, 1.4 Hz, 2H), 7.27 (m, 1H), 7.18 (m, 1H) , 7.14 (m, 1H), 6.58-6.46 (m, 3H), 4.59 (s, 2H), 4.46 (d, J = 13.4 Hz, 2H), 4.23 (s, 2H), 3.37 (s, 1H), 3.18 (t, J = 12.7 Hz, 2H), 2.34- 2.25 (m, 2H), 1.89-1.75 (m, 2H). | 619.2 |
| 132 | (E)-3-(4-(((1-(3- Cyano-4-(4- cyano-3- fluorophenyl)-5- (3-hydroxy-4- methoxyphenyl) pyridin-2- yl)piperidin-4- yl)amino)methyl)- 3- methoxyphenyl)- N- hydroxyacrylamide formate | 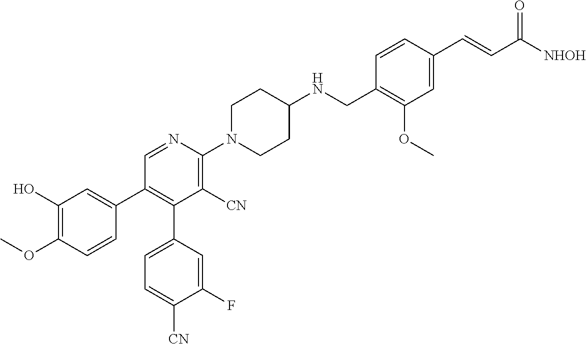 | ¹H NMR (400 MHz, DMSO-d6) δ 10.71 (s, 1H), 9.00 (s, 1H), 8.41 (s, 1H), 8.19 (s, 1H), 7.95 (t, J = 7.4 Hz, 1H), 7.66 (d, J = 10.0 Hz, 1H), 7.50-7.38 (m, 2H), 7.29-7.24 (m, 1H), 7.20-7.10 (m, 2H), 6.80 (d, J = 8.0 Hz, 1H), 6.53-6.40 (m, 3H), 4.18 (dt, J = 13.2, 3.8 Hz, 2H), 3.85 (s, 3H), 3.80 (s, 2H), 3.71 (s, 3H), 3.17 (s, 2H), 2.84- 2.71 (m, 1H), 2.05-1.92 (m, 2H), 1.47 (d, J = 11.0 Hz, 2H). | 649.3 |
| 133 | (E)-3-(5-(((1-(3- Cyano-4-(4- cyano-3- fluorophenyl)-5- (3-hydroxy-4- methoxyphenyl) pyridin-2- yl)piperidin-4- yl)amino)methyl) pyrazin-2-yl)-N- hydroxyacrylamide trifluoroacetate | 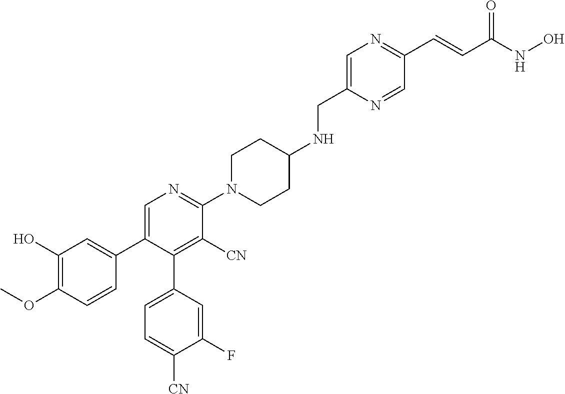 [SMILES: COc1ccc(-c2cnc(N3CCC(NCc4cnc(/C=C/C(=O)NO)cn4)CC3)c(C#N)c2-c2ccc(C#N)c(F)c2)cc1O] | ¹H NMR (400 MHz, DMSO-d6) δ ppm: δ 11.04 (s, 1H), 9.24 (s, 1H), 9.03 (s, 1H), 8.94 (s, 1H), 8.82 (s, 1H), 8.46 (s, 1H), 7.97 (t, J = 7.4 Hz, 1H), 7.71-7.60 (m, 2H), 7.28 (dd, J = 8.0, 1.4 Hz, 1H), 7.07 (d, J = 15.4 Hz, 1H), 6.82 (d, J = 8.1 Hz, 1H),6.46 (s, 2H), 4.54 (s, 2H), 4.32 (d, J = 13.0 Hz, 2H), 3.72 (s, 3H), 3.50 (s, 1H), 3.13 (t, J = 12.5 Hz, 2H), 2.29-2.20 (m, 2H), 1.85-1.71 (m, 2H). | 621.2 |
| 134 | (E)-3-(4-(((1-(3- Cyano-4-(4- cyano-2- fluorophenyl)-5- (3-hydroxy-4- methoxyphenyl) pyridin-2- yl)piperidin-4- yl)amino)methyl) phenyl)-N- hydroxyacrylamide formate | 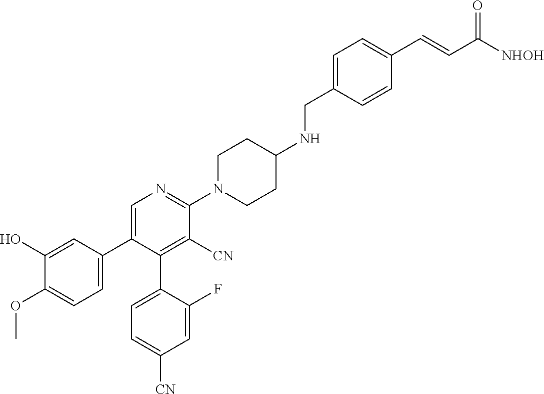 | ¹H NMR (400 MHz, DMSO-d6) δ 10.80 (s, 1H), 9.04 (s, 2H), 8.48 (s, 1H), 7.97 (d, J = 9.6 Hz, 1H), 7.80 (d, J = 8.0 Hz, 1H), 7.68-7.60 (m, 3H), 7.57 (d, J = 8.0 Hz, 2H), 7.47 (d, J = 15.6 Hz, 1H), 6.80 (d, J = 8.4 Hz, 1H), 6.59-6.39 (m, 3H), 4.37-4.11 (m, 4H), 3.71 (s, 3H), 3.27 (s, 1H),3.14 (m, 2H), 2.22 (d, J = 12.4 Hz, 2H), 1.73 (s, 2H). | 619.2 |
| 135 | (E)-3-(2-(((1-(3- Cyano-4-(4- cyano-3- fluorophenyl)-5- (3-hydroxy-4- methoxyphenyl) pyridin-2- yl)piperidin-4- yl)amino)methyl) pyrimidin-5-yl)-N- hydroxyacrylamide formate | 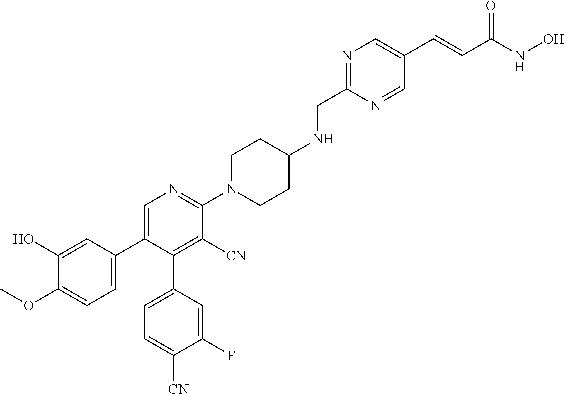 [SMILES: COc1ccc(-c2cnc(N3CCC(NCc4ncc(/C=C/C(=O)NO)cn4)CC3)c(C#N)c2-c2ccc(C#N)c(F)c2)cc1O] | ¹H NMR (400 MHz, Methanol-d4) δppm 9.02 (s, 2H), 8.52 (s, 1H), 8.11 (d, J = 9.6 Hz, 1H), 7.74 (t, J = 7.2 Hz, 1H), 7.58 (d, J = 15.8 Hz, 1H), 7.37 (d, J = 9.6 Hz, 1H), 7.23 (d, J = 7.8 Hz, 1H), 6.81 (d, J = 8.4 Hz, 1H), 6.72 (d, J = 15.8 Hz, 1H), 6.51 (d, J = 6.5 Hz, 2H), 4.46 (d, J = 5.8 Hz, 3H), 4.42 (s, 1H), 3.81 (s, 3H), 3.37 (d, J = 11.8 Hz, 1H), 3.18 (t, J = 12.6 Hz, 2H), 2.28 (d, J = 12.0 Hz, 2H), 1.93-1.71 (m, 2H). | 621.2 |
| 136 | (E)-3-(4-(((1-(3- Cyano-4-(4- cyano-3- fluorophenyl)-5- (1-methyl-1H- benzo[d]imidazol- 5-yl))pyridin-2- yl)piperidin-4- yl)amino)methyl) phenyl)-N- hydroxyacrylamide formate | 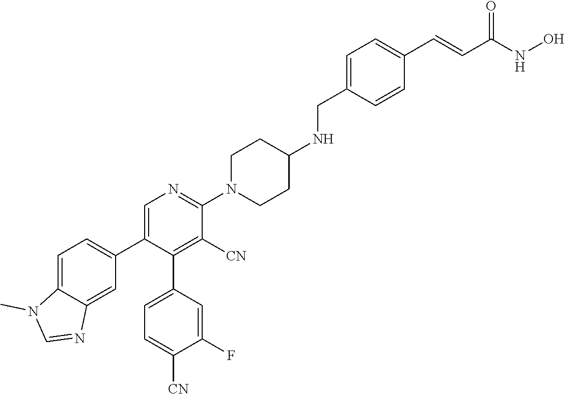 [SMILES: Cn1cnc2cc(-c3cnc(N4CCC(NCc5ccc(/C=C/C(=O)NO)cc5)CC4)c(C#N)c3-c3ccc(C#N)c(F)c3)ccc21] | ¹H NMR (400 MHz, MeOD) δppm 8.52 (d, J = 3.6 Hz, 1H), 8.12 (s, 1H), 7.67 (t, J = 7.2 Hz, 1H), 7.61 (d, J = 7.8 Hz, 2H), 7.58-7.48 (m, 2H), 7.46 (d, J = 8.4 Hz, 3H), 7.43- 7.35 (m, 2H), 7.24 (d, J = 7.8 Hz, 1H), 7.05 (d, J = 8.4 Hz, 1H), 6.50 (d, J = 15.8 Hz, 1H), 4.46 (d, J = 13.2 Hz, 2H), 4.10 (s, 2H), 3.87 (s, 3H), 3.23-3.13 (m, 3H), 2.26- 2.19 (m, 2H), 1.75 (td, J = 13.1, 9.4 Hz, 2H). | 627.3 |
| 137 | (E)-3-(4-(((1-(3- Cyano-4-(4- cyano-3- fluorophenyl)-5- (3,4- dihydroxyphenyl) pyridin-2- yl)piperidin-4- yl)amino)methyl) phenyl)-N- hydroxyacrylamide hydrochloride | 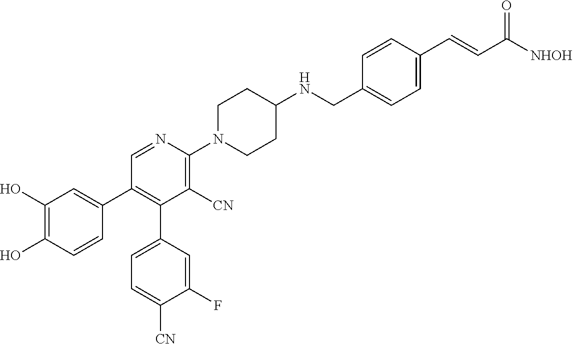 | ¹H NMR (400 MHz, DMSO-d6) δppm: 10.78 (s, 1H), 9.03 (d, J = 8.4 Hz, 2H), 8.92 (s, 1H), 8.43 (s, 1H), 7.96 (t, J = 7.6 Hz, 1H), 7.71- 7.51 (m, 5H), 7.47 (d, J = 15.8 Hz, 1H), 7.27 (d, J = 8.4 Hz, 1H), 6.61 (d, J = 8.0 Hz, 1H), 6.50 (d, J = 16.0 Hz, 1H), 6.41 (d, J = 2.4 Hz, 1H), 6.33 (dd, J = 8.0, 2.1 Hz, 1H), 4.27 (d, J = 12.8 Hz, 2H), 4.14 (s, 2H), 3.13 (t, J = 12.4 Hz, 2H), 2.49 (s, 1H), 2.19 (d, J = 11.6 Hz, 2H), 1.70 (s, 2H). | 605.2 |
| 138 | (E)-3-(4-(((7-(3- Cyano-4-(4- cyano-3- fluorophenyl)-5- (3-hydroxy-4- methoxyphenyl) pyridin-2-yl)-2,7- diazaspiro[3.5] nonan-2- yl)methyl)phenyl)- N- hydroxyacrylamide formate | 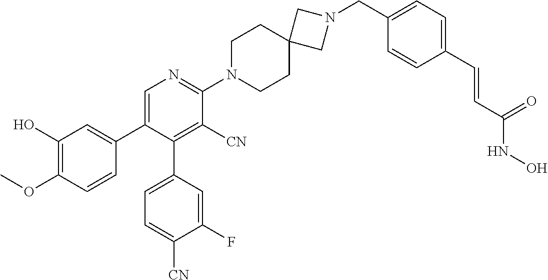 [SMILES: COc1ccc(-c2cnc(N3CCC4(CC3)CN(Cc3ccc(/C=C/C(=O)NO)cc3)C4)c(C#N)c2-c2ccc(C#N)c(F)c2)cc1O] | ¹H NMR (400 MHz, DMSO-d6) δ ppm: 10.73 (s, 1H), 8.99 (s, 1H), 8.41 (s, 1H), 8.14 (d, J = 3.0 Hz, 1H), 7.95 (t, J = 7.4 Hz, 1H), 7.65 (d, J = 10.0 Hz, 1H), 7.52 (d, J = 7.8 Hz, 2H), 7.44 (d, J = 15.6 Hz, 1H), 7.35 (d, J = 7.8 Hz, 2H), 7.27 (d, J = 8.0 Hz, 1H), 6.80 (d, J = 8.2 Hz, 1H), 6.45 (s, 3H), 3.71 (s, 5H), 3.59 (d, J = 11.0 Hz, 4H), 3.15 (s, 4H), 1.85 (t, J = 5.3 Hz, 4H). | 645.3 |
| 139 | (E)-3-(4-(((1-(3- Cyano-4-(4- cyano-3- fluorophenyl)-5- (3- hydroxyphenyl) pyridin-2- yl)piperidin-4- yl)amino)methyl) phenyl)-N- hydroxyacrylamide formate | 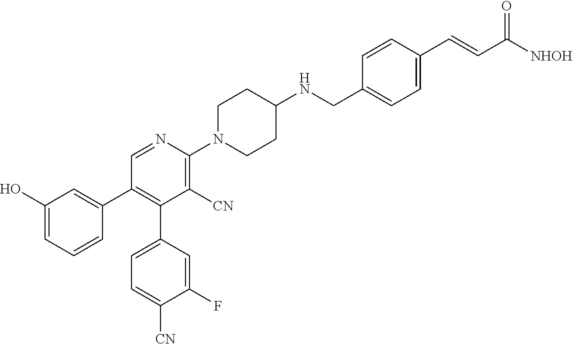 | ¹H NMR (400 MHz, DMSO-d6) δ ppm: 10.72 (s, 1H), 9.42 (s, 1H), 8.44 (s, 1H), 8.16 (d, J = 1.6 Hz, 1H), 7.95 (t, J = 7.4 Hz, 1H), 7.65 (d, J = 10.0 Hz, 1H), 7.52 (d, J = 7.8 Hz, 2H), 7.44 (t, J = 8.6 Hz, 3H), 7.29 (d, J = 8.0 Hz, 1H), 7.04 (t, J = 7.8 Hz, 1H), 6.63 (dd, J = 8.2, 2.2 Hz, 1H), 6.46 (s, 3H), 4.21 (dt, J = 13.4, 4.0 Hz, 2H), 3.85 (s, 2H), 3.18 (t, J = 11.8 Hz, 2H), 2.79 (s, 1H), 2.01 (dd, J = 13.2, 3.8 Hz, 2H), 1.48 (q, J = 10.4 Hz, 2H). | 589.2 |
| 140 | (E)-3-(4-(((1-(3- Cyano-4-(4- cyano-3- fluorophenyl)-5- (2,3-dihydroxy-4- methoxyphenyl) pyridin-2- yl)piperidin-4- yl)amino)methyl) phenyl)-N- hydroxyacrylamide formate | 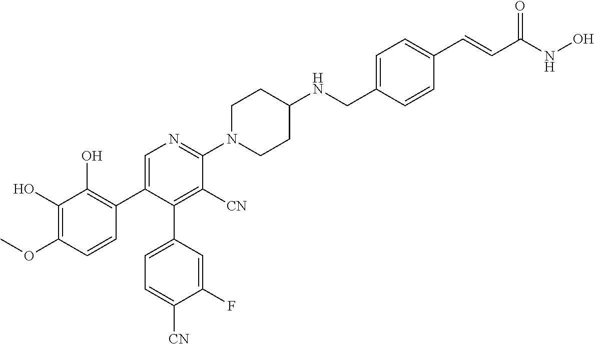 [SMILES: COc1ccc(-c2cnc(N3CCC(NCc4ccc(/C=C/C(=O)NO)cc4)CC3)c(C#N)c2-c2ccc(C#N)c(F)c2)c(O)c1O] | ¹H NMR (400 MHz, Methanol-d4) δ ppm: 8.52 (s, 1H), 8.38 (s, 1H), 7.80-7.40 (m, 6H), 7.32 (d, J = 9.8 Hz, 1H), 7.22 (d, J = 8.0 Hz, 1H), 6.54 (d, J = 15.8 Hz, 1H), 6.43 (s, 2H), 4.44 (d, J = 13.2 Hz, 2H), 4.31 (d, J = 10.6 Hz, 2H), 3.81 (s, 3H), 3.51 (s, 1H), 3.20 (d, J = 12.8 Hz, 2H), 2.34 (d, J = 11.8 Hz, 2H), 1.89 (d, J = 12.4 Hz, 2H). | 635.2 |
| 141 | (E)-3-(4-(((1-(3- Cyano-4-(4- cyano-3- fluorophenyl)-5- (1-methyl-1H- benzo[d][1,2,3] triazol-5- yl)pyridin-2- yl)piperidin-4- yl)amino)methyl) phenyl)-N- hydroxyacrylamide formate | 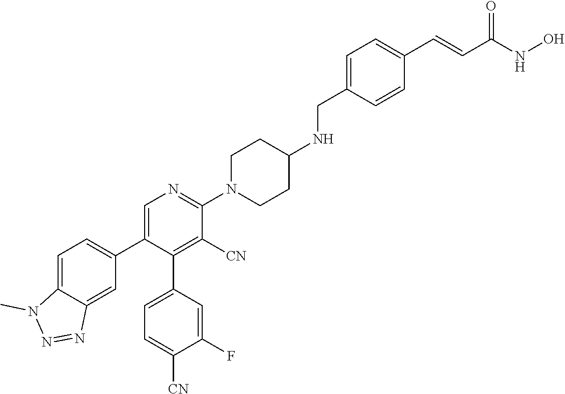 [SMILES: Cn1nnc2cc(-c3cnc(N4CCC(NCc5ccc(/C=C/C(=O)NO)cc5)CC4)c(C#N)c3-c3ccc(C#N)c(F)c3)ccc21] | ¹H NMR (400 MHz, DMSO-d6) δ 10.73 (s, 1H), 9.02 (s, 1H), 8.58 (s, 1H), 7.89 (s, 2H), 7.70 (dd, J = 11.2, 9.0 Hz, 2H), 7.55 (d, J = 7.8 Hz, 2H), 7.50-7.41 (m, 3H), 7.33 (d, J = 8.0 Hz, 1H), 7.14 (dd, J = 8.4, 1.4 Hz, 1H), 6.45 (d, J = 2.8 Hz, 1H), 4.26 (s, 5H), 3.92 (s, 2H), 3.21 (t, J = 12.0 Hz, 2H), 2.90 (s, 1H), 2.07 (d, J = 6.0 Hz, 2H), 1.54 (d, J = 11.4 Hz, 2H). | 628.3 |
| 142 | (E)-3-(4-(((1-(3- Cyano-4-(4- cyano-3- fluorophenyl)-5- (1H-indazol-6- yl)pyridin-2- yl)piperidin-4- yl)amino)methyl) phenyl)-N- hydroxyacrylamide formate | 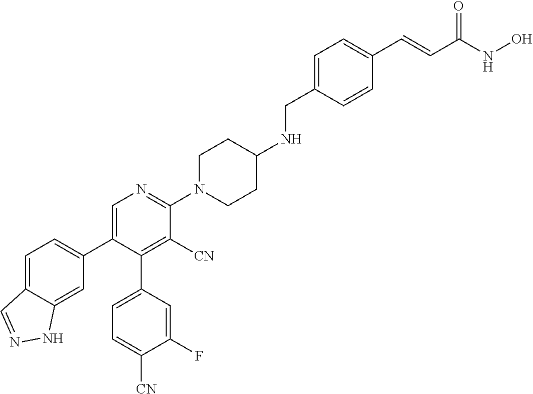 [SMILES: N#Cc1ccc(-c2c(-c3ccc4cn[nH]c4c3)cnc(N3CCC(NCc4ccc(/C=C/C(=O)NO)cc4)CC3)c2C#N)cc1F] | ¹H NMR (400 MHz, DMSO-d6) δ 13.06 (s, 1H), 10.72 (s, 1H), 9.01 (s, 1H), 8.56 (s, 1H), 8.01 (s, 1H), 7.89 (t, J = 7.4 Hz, 1H), 7.71 (d, J = 10.0 Hz, 1H), 7.56 (dd, J = 8.6, 8.2 Hz, 3H), 7.44 (t, J = 8.2 Hz, 3H), 7.30 (d, J = 7.2 Hz, 2H), 6.73 (d, J = 8.4 Hz, 1H), 6.44 (d, J = 5.8 Hz, 1H), 4.24 (d, J = 13.2 Hz, 2H), 3.86 (s, 2H), 3.21 (t, J = 11.8 Hz, 2H), 2.81 (s, 1H), 2.03 (d, J = 12.2 Hz, 2H), 1.50 (q, J = 11.4, 10.8 Hz, 2H). | 613.2 |
| 143 | (E)-3-(4-(((1-(3- Cyano-4-(4- cyano-3- fluorophenyl)-5- (3-hydroxy-4-(2- hydroxy-2- methylpropoxy) phenyl)pyridin-2- yl)piperidin-4- yl)amino)methyl) phenyl)-N- hydroxyacrylamide formate | 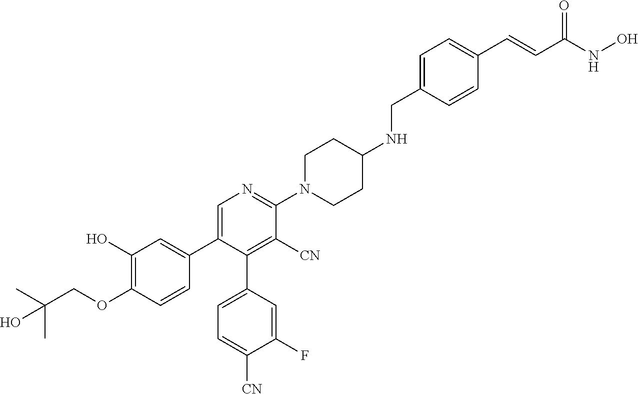 [SMILES: CC(C)(O)COc1ccc(-c2cnc(N3CCC(NCc4ccc(/C=C/C(=O)NO)cc4)CC3)c(C#N)c2-c2ccc(C#N)c(F)c2)cc1O] | ¹H NMR (400 MHz, Methanol-d4) δppm 8.52(s, 1H)8.44 (s, 1H), 7.74 (t, J = 7.4 Hz, 1H), 7.70-7.60 (m, 3H), 7.56 (d, J = 9.6 Hz, 2H), 7.36 (d, J = 9.6 Hz, 1H), 7.23 (d, J = 7.8 Hz, 1H), 6.80 (d, J = 8.4 Hz, 1H), 6.55 (d, J = 2.2 Hz, 1H), 6.52- 6.44 (m, 2H), 4.45 (d, J = 13.2 Hz, 2H), 4.28 (s, 2H), 3.78 (s, 2H), 3.46 (d, J = 15.4 Hz, 1H), 3.17 (t, J = 12.4 Hz, 2H), 2.32 (d, J = 11.6 Hz, 2H), 1.85 (d, J = 11.6 Hz, 2H), 1.30 (s, 6H). | 677.3 |
| 144 | (E)-3-(4-(((1-(4- (4-Cyano-3- fluorophenyl)-5- (3-hydroxy-4- methoxyphenyl)- 3-methylpyridin- 2-yl)piperidin-4- yl)amino)methyl) phenyl)-N- hydroxyacrylamide hydrochloride | 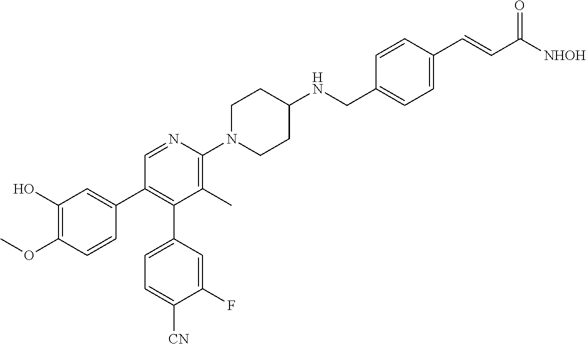 | ¹H NMR (400 MHz, DMSO-d6) δ10.75 (s, 1H), 8.89 (s, 1H), 8.18 (d, J = 2.4 Hz, 1H), 8.10 (s, 1H), 7.87 (t, J = 7.6 Hz, 1H), 7.53 (d, J = 7.6 Hz, 2H), 7.50-7.33 (m, 4H), 7.10 (dd, J = 8.0, 1.6 Hz, 1H), 6.76 (d, J = 8.8 Hz, 1H), 6.43 (m, 3H), 3.87 (s, 2H), 3.70 (s, 3H), 3.46 (s, 2H), 2.77 (m, 3H), 1.97 (s, 5H), 1.52 (s, 2H). | 608.3 |
| 145 | (E)-3-(5-(((1-(3- Cyano-4-(4- cyano-3- fluorophenyl)-5- (4- (difluoromethoxy)- 3- hydroxyphenyl) pyridin-2- yl)piperidin-4- yl)amino)methyl) pyrimidin-2-yl)-N- hydroxyacrylamide formate | 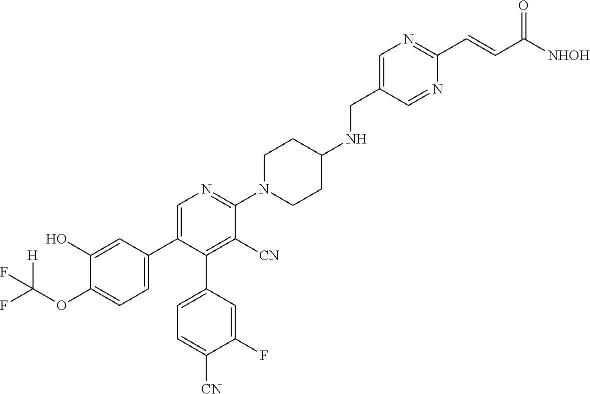 | ¹H NMR (400 MHz, Methanol-d4) δppm 8.95 (s, 2H), 8.46 (s, 1H), 7.76 (t, J = 7.4 Hz, 1H), 7.55 (d, J = 15.4 Hz, 1H), 7.41 (d, J = 9.6 Hz, 1H), 7.33-7.17 (m, 2H), 7.01 (d, J = 8.0 Hz, 2H), 6.64 (s, 1H), 6.60- 6.50 (m, 1H), 4.60-4.32 (m, 4H), 3.72-3.47 (m, 1H), 3.22 (t, J = 12.4 Hz, 2H), 2.37 (s, 2H), 1.92 (d, J = 13.8 Hz, 2H). | 657.2 |
| 147 | (E)-3-(4-(4-(4- Cyano-3- fluorophenyl)-5- (3-hydroxy-4- methoxyphenyl)- 3- (trifluoromethyl) pyridin-2- yl)piperidin-4- yl)amino)methyl) phenyl)-N- hydroxyacrylamide formate | 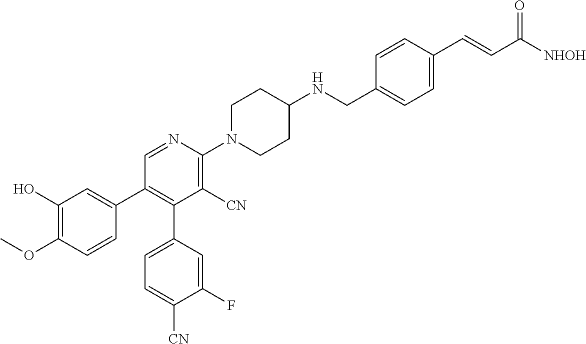 | ¹H NMR (400 MHz, Methanol-d4) δppm: 8.52 (s, 1H),8.41 (s, 1H), 7.87 (dd, J = 8.0, 6.2 Hz, 1H), 7.75- 7.37 (m, 7H), 7.09 (d, J = 8.0 Hz, 1H), 6.96-6.83 (m, 2H), 6.51 (d, J = 5.8 Hz, 1H), 4.16 (s, 2H), 3.93 (s, 3H), 3.41 (d, J = 6.2 Hz, 2H), 3.23 (s, 1H), 2.92 (t, J = 12.0 Hz, 2H), 2.00-1.92 (m, 2H), 1.62 (d, J = 13.6 Hz, 2H). | 662.2 |
| 148 | (E)-3-(5-(((1-(3- Cyano-4-(4- cyano-3- fluorophenyl)-5- (3-hydroxy-4- methylphenyl) pyridin-2- yl)piperidin- 4- yl)amino)methyl) pyrimidin-2-yl)-N- hydroxyacrylamide formate | 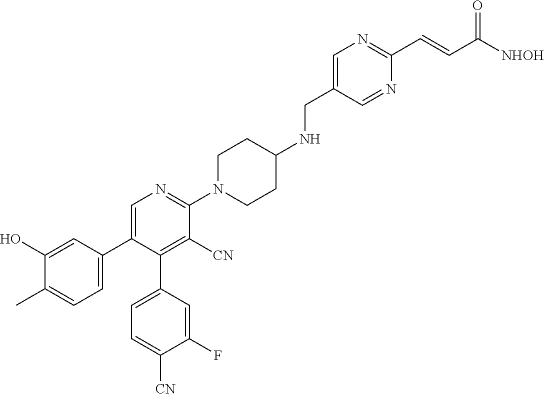 | ¹H NMR (400 MHz, DMSO-d6)δ ppm: 11.10 (s, 1H), 9.31 (s, 1H), 8.99 (s, 2H), 8.45 (s, 1H), 8.28 (s, 1H), 7.96 (t, J = 7.4 Hz, 1H), 7.70 (d, J = 10.0 Hz, 1H), 7.38 (d, J = 15.4 Hz, 1H), 7.26 (dd, J = 7.8, 1.4 Hz, 1H), 7.19 (d, J = 15.6 Hz, 1H), 6.95 (d, J = 7.8 Hz, 1H), 6.42 (d, J = 1.8 Hz, 2H), 4.30 (s, 2H), 4.22 (s, 1H), 3.31 (s, 2H),3.17 (s, 2H), 2.26 (s, 2H),2.04 (s, 3H), 1.74 (d, J = 13.8 Hz, 2H). | 604.2 |
| 149 | (E)-3-(4-(((1-(3- Cyano-4-(4- cyano-3- fluorophenyl)-5- (3-hydroxy-4- methoxyphenyl) pyridin-2-yl)azepan- 4- yl)amino)methyl) phenyl)-N- hydroxyacrylamide formate | 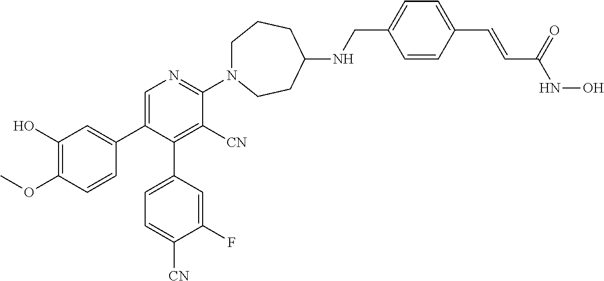 [SMILES: COc1ccc(-c2cnc(N3CCCC(NCc4ccc(/C=C/C(=O)NO)cc4)CC3)c(C#N)c2-c2ccc(C#N)c(F)c2)cc1O] | ¹H NMR (400 MHz, DMSO-d6)δ ppm: 10.71 (s, 1H), 8.96 (s, 1H), 8.49 (s, 1H), 8.21-8.13 (m, 1H), 7.94 (t, J = 7.4 Hz, 1H), 7.61 (d, J = 7.6 Hz, 1H), 7.51 (d, J = 7.8 Hz, 2H), 7.48-7.37 (m, 2H), 7.25 (s, 1H), 6.82-6.75 (m, 1H), 6.49- 6.39 (m, 3H), 4.02-3.73 (m, 2H), 3.79 (s, 2H), 3.75 (s, 1H),3.71 (s, 3H), 3.69 (s, 1H), 2.76 (s, 1H), 2.19 (s, 1H),2.10 (s, 1H), 1.88 (d, J = 6.2 Hz, 1H), 1.76 (d, J = 10.0 Hz, 2H), 1.51 (t, J = 11.2 Hz, 1H). | 632.2 |
| 150 | (E)-3-(4-(((1-(3- Cyano-5-(3- hydroxy-4- methoxyphenyl)- 4-(4- nitrophenyl) pyridin-2- yl)piperidin-4- yl)amino)methyl) phenyl)-N- hydroxyacrylamide formate | 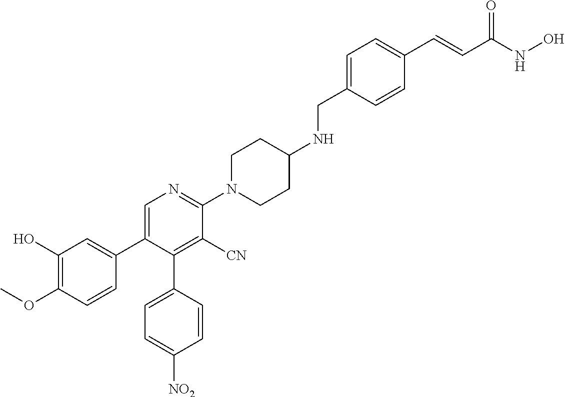 [SMILES: COc1ccc(-c2cnc(N3CCC(NCc4ccc(/C=C/C(=O)NO)cc4)CC3)c(C#N)c2-c2ccc([N+](=O)[O-])cc2)cc1O] | ¹H NMR (400 MHz, Methanol-d4) δppm 8.50 (s, 1H), 8.43 (s, 1H), 8.26-8.12 (m, 2H), 7.71-7.55 (m, 3H), 7.55-7.42 (m, 4H), 6.78 (d, J = 8.2 Hz, 1H), 6.62-6.34 (m, 3H), 4.42 (d, J = 13.4 Hz, 2H), 4.17 (s, 2H), 3.78 (s, 3H), 3.30 (s, 1H), 3.22-3.07 (m, 2H), 2.38-2.20 (m, 2H), 1.88-1.70 (m, 2H). | 621.2 |
| 151 | (E)-3-(2-(((1-(3- Cyano-4-(4- cyano-3- fluorophenyl)-5- (3-hydroxy-4- methoxyphenyl) pyridin-2- yl)piperidin-4- yl)amino)methyl) thiazol-5-yl)-N- hydroxyacrylamide hydrochloride | 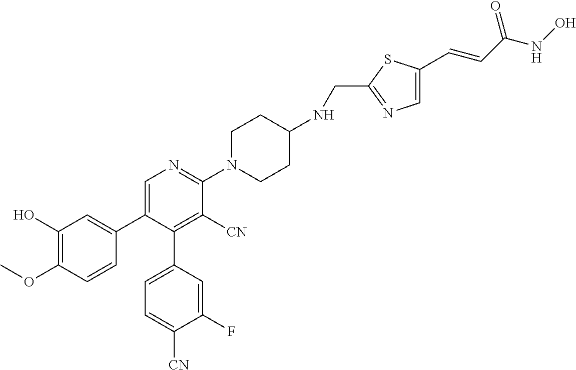 [SMILES: COc1ccc(-c2cnc(N3CCC(NCc4ncc(/C=C/C(=O)NO)s4)CC3)c(C#N)c2-c2ccc(C#N)c(F)c2)cc1O] | ¹H NMR (400 MHz, DMSO-d6) δ 10.68 (s, 1H), 9.01 (s, 2H), 8.42 (s, 1H), 7.99-7.89 (m, 2H), 7.70- 7.56 (m, 2H), 7.27 (dd, J = 8.0, 1.6 Hz, 1H), 6.80 (d, J = 8.4 Hz, 1H), 6.46 (s, 2H), 6.16 (d, J = 15.6 Hz, 1H), 4.18 (d, J = 13.2 Hz, 2H), 4.05 (s, 2H), 3.71 (s, 3H), 3.17 (t, J = 12.4 Hz, 2H), 2.80-2.75 (m, 1H), 1.99 (d, J = 12. 4Hz, 2H), 1.45 (q, J = 10.8, 10.4 Hz, 2H). | 626.2 |
| 152 | 3-(5-((1-(3-Cyano- 4-(4-cyano-3- fluorophenyl)-5- (3-hydroxy-4- methoxyphenyl) pyridin-2- yl)piperidin-4- yl)amino)methyl) pyrimidin-2-yl)-N- hydroxypropanamide hydrochloride | 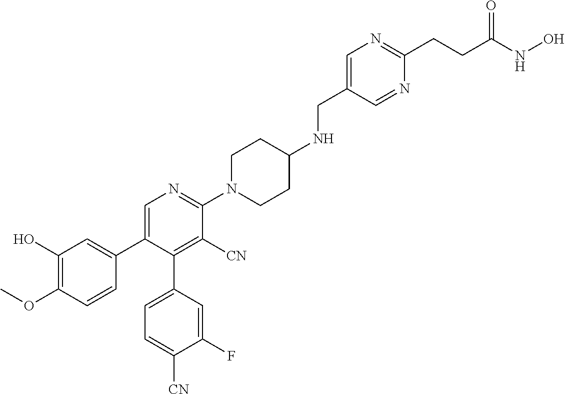 [SMILES: COc1ccc(-c2cnc(N3CCC(NCc4cnc(CCC(=O)NO)nc4)CC3)c(C#N)c2-c2ccc(C#N)c(F)c2)cc1O] | ¹H NMR (400 MHz, DMSO-d6) δ 10.42 (s, 1H), 8.98 (s, 1H), 8.68 (s, 2H), 8.41 (s, 1H), 8.14 (s, 1H), 7.95 (dd, J = 8.0, 6.8 Hz, 1H), 7.66 (dd, J = 10.4, 1.5 Hz, 1H), 7.27 (dd, J = 8.0, 1.6 Hz, 1H), 6.80 (d, J = 8.0 Hz, 1H), 6.59-6.38 (m, 2H), 4.27-4.10 (m, 2H), 3.79 (s, 2H), 3.71 (s, 3H), 3.25-3.14 (m, 2H), 3.08 (t, J = 7.6 Hz, 2H), 2.74 (s, 1H), 2.46 (d, J = 7.6 Hz, 2H), 2.00 (d, J = 12.8 Hz, 2H), 1.45 (q, J = 10.4 Hz, 2H). | 623.3 |
| 153 | (E)-3-(4-(((1-(3- Cyano-4-(4- cyano-3- fluorophenyl)-5- (4- (dimethylamino)- 3- hydroxyphenyl) pyridin-2- yl)piperidin-4- yl)amino)methyl) phenyl)-N- hydroxyacrylamide formate | 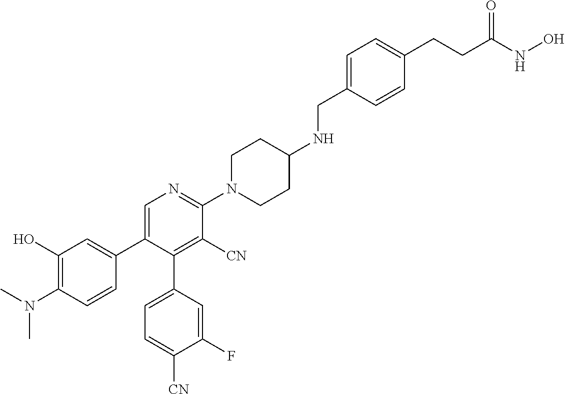 [SMILES: CN(C)c1ccc(-c2cnc(N3CCC(NCc4ccc(CCC(=O)NO)cc4)CC3)c(C#N)c2-c2ccc(C#N)c(F)c2)cc1O] | ¹H NMR (400 MHz, DMSO) δppm: 10.79 (s, 1H), 8.94 (s, 2H), δ 8.50 (s, 1H), 7.96 (dd, J = 8.0, 6.8 Hz, 1H), 7.75-7.62 (m, 3H), 7.57 (d, J = 8.0 Hz, 2H), 7.53-7.45 (m, 2H), 7.30 (dd, J = 8.0, 1.4 Hz, 1H), 6.76 (dd, J = 8.4, 2.0 Hz, 1H), 6.71 (d, J = 1.8 Hz, 1H), 6.54 (d, J = 15.8 Hz, 1H), 4.38 (d, J = 12.9 Hz, 2H), 4.26 (s, 2H), 3.23 (m, 1H), 3.16 (t, J = 12.7 Hz, 2H), 3.09 (s, 6H), 2.28 (d, J = 12.0 Hz, 2H), 1.84- 1.67 (m, 2H). | 632.3 |
| 154 | (E)-3-(4-(((1-(3- Cyano-4-(4- cyano-3- fluorophenyl)-5- (3-hydroxy-4- methoxyphenyl) pyridin-2- yl)piperidin-4- yl)amino)methyl)- 3-fluorophenyl)- N- hydroxyacrylamide hydrochloride | 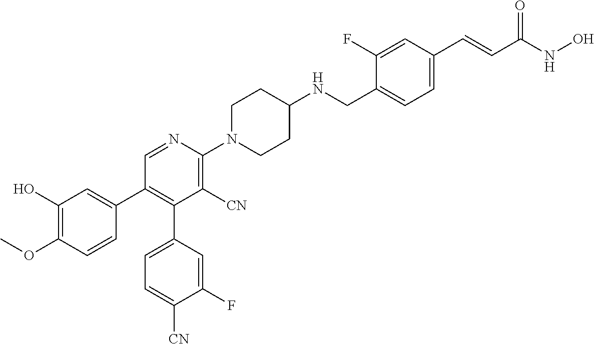 [SMILES: COc1ccc(-c2cnc(N3CCC(NCc4ccc(/C=C/C(=O)NO)cc4F)CC3)c(C#N)c2-c2ccc(C#N)c(F)c2)cc1O] | ¹H NMR (400 MHz, DMSO-d6) δ 10.76 (s, 1H), 8.99 (s, 1H), 8.41 (s, 1H), 8.15 (s, 1H), 7.95 (dd, J = 8.0, 6.8 Hz, 1H), 7.66 (dd, J = 10.4, 1.6 Hz, 1H), 7.55 (t, J = 7.6 Hz, 1H), 7.48-7.34 (m, 3H), 7.27 (dd, J = 8.0, 1.6 Hz, 1H), 6.80 (d, J = 8.4 Hz, 1H), 6.56-6.39 (m, 3H), 4.29- 4.10 (m, 2H), 3.86 (s, 2H), 3.71 (s, 3H), 3.18 (t, J = 11.6 Hz, 1H), 2.13-1.93 (m, 2H), 2.78-2.76 (m, 2H), 1.47 (q, J = 10.4 Hz, 2H). | 637.2 |
| 155 | (E)-3-(4-(((1-(3- Cyano-4-(4- cyano-3- fluorophenyl)-5- (3-hydroxy-4- methoxyphenyl) pyridin-2- yl)piperidin-4- yl)amino)methyl) cyclohex-1-en-1- yl)-N- hydroxyacrylamide formate | 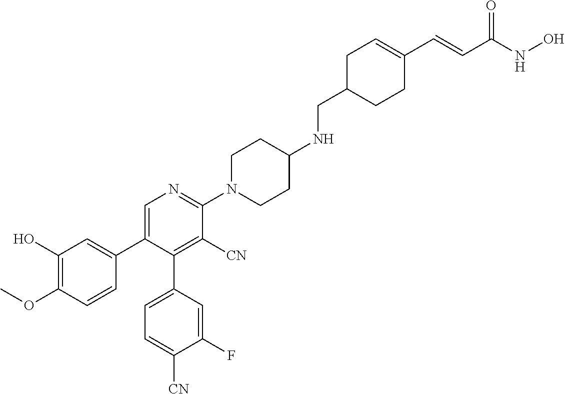 [SMILES: COc1ccc(-c2cnc(N3CCC(NCC4CC=C(/C=C/C(=O)NO)CC4)CC3)c(C#N)c2-c2ccc(C#N)c(F)c2)cc1O] | ¹H NMR (400 MHz, Methanol-d4) δppm 8.44 (s, 1H), 8.33 (s, 1H), 7.64 (dd, J = 8.0, 6.6 Hz, 1H), 7.26 (dd, J = 9.8, 1.2 Hz, 1H), 7.17- 7.06 (m, 2H), 6.75-6.68 (m, 1H), 6.44-6.38 (m, 2H), 6.04 (s, 1H), 5.71 (d, J = 15.6 Hz, 1H), 4.33 (d, J = 13.2 Hz, 2H), 3.71 (s, 3H), 3.20 (d, J = 1.6 Hz, 1H), 3.12-3.00 (m, 2H), 2.88 (d, J = 5.6 Hz, 2H), 2.35 (d, J = 16.4 Hz, 1H), 2.25 (d, J = 18.0 Hz, 1H), 2.12 (td, J = 15.8, 15.0, 5.0 Hz, 3H), 1.96-1.87 (m, 3H), 1.75-1.61 (m, 2H), 1.45- 1.23 (m, 1H). | 623.3 |
| 156 | (E)-3-(3-Chloro-4- ((1-(3-cyano-4-(4- cyano-3- fluorophenyl)-5- (3-hydroxy-4- methoxyphenyl) pyridin-2- yl)piperidin-4- yl)amino)methyl) phenyl)-N- hydroxyacrylamide formate | 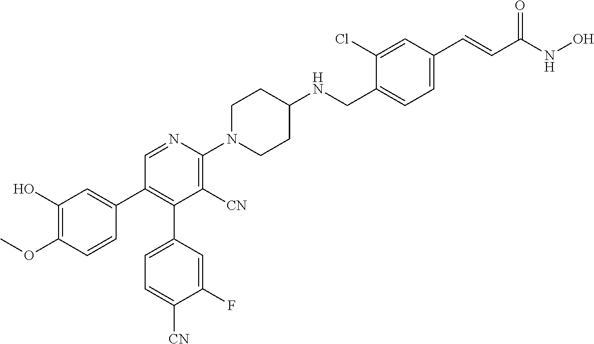 [SMILES: COc1ccc(-c2cnc(N3CCC(NCc4ccc(/C=C/C(=O)NO)cc4Cl)CC3)c(C#N)c2-c2ccc(C#N)c(F)c2)cc1O] | ¹H NMR (400 MHz, DMSO-d6) δ ppm: 10.77 (s, 1H), 9.00 (s, 1H), 8.43 (s, 1H), 8.12 (s, 1H), 7.96 (t, J = 7.4 Hz, 1H), 7.66 (dd, J = 5.8, 3.8 Hz, 3H), 7.56 (d, J = 8.0 Hz, 1H), 7.43 (d, J = 15.8 Hz, 1H), 7.28 (dd, J = 8.2, 1.4 Hz, 1H), 6.81 (d, J = 8.2 Hz, 1H), 6.57-6.46 (m, 3H), 4.22 (d, J = 13.2 Hz, 2H), 4.00 (s, 2H), 3.71 (s, 3H), 3.24-3.12 (m, 2H), 2.95 (s, 1H), 2.08 (d, J = 12.0 Hz, 2H), 1.56 (d, J = 11.4 Hz, 2H). | 653.2 |
| 158 | (E)-3-(4-(((1-(3- Cyano-4-(4- cyano-3- fluorophenyl)-5- (2,5-dihydroxy-4- methoxyphenyl) pyridin-2- yl)piperidin-4- yl)amino)methyl) phenyl)-N- hydroxyacrylamide trifluoroacetate | 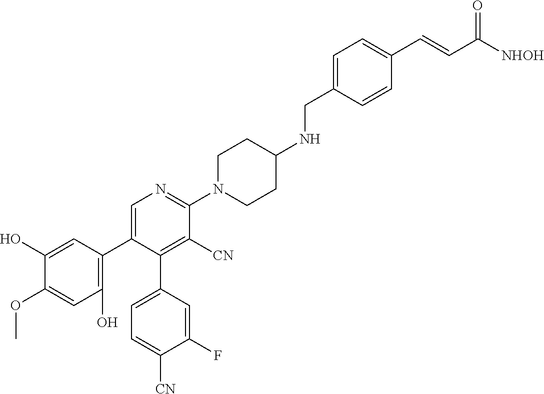 | ¹H NMR (400 MHz, Methanol-d4) δ 8.35 (s, 1H), 7.73-7.62 (m, 3H), 7.59-7.46 (m, 3H), 7.33 (dd, J = 9.8, 1.4 Hz, 1H), 7.22 (dd, J = 8.0, 1.4 Hz, 1H), 6.52 (d, J = 5.8 Hz, 1H), 6.43 (s, 1H), 6.31 (s, 1H), 4.42 (d, J = 3.6 Hz, 2H), 4.25 (s, 2H), 3.76 (s, 3H), 3.59-3.46 (m, 1H),3.16 (s, 2H), 2.29 (d, J = 8.4 Hz, 2H), 1.90-1.76 (m, 2H). | 635.2 |
| 159 | (E)-3-(5-(((1-(5- (4-Chloro-3- hydroxyphenyl)-3- cyano-4-(4-cyano- 3- fluorophenyl) pyridin-2- yl)piperidin- 4- yl)amino)methyl) pyrimidin-2-yl)-N- hydroxyacrylamide hydrochloride | 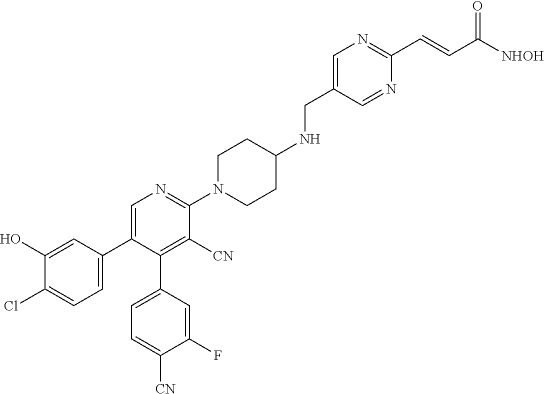 | ¹H NMR (400 MHz, Methanol-d4) δ ppm 8.90 (s, 2H), 8.45 (s, 1H), 7.76 (dd, J = 8.0, 6.6 Hz, 1H), 7.57 (d, J = 15.4 Hz, 1H), 7.42 (dd, J = 9.6, 1.5 Hz, 1H), 7.29-7.16 (m, 3H), 6.62 (d, J = 2.2 Hz, 1H), 6.54 (dd, J = 8.2, 2.0 Hz, 1H), 4.47 (d, J = 13.2 Hz, 2H), 4.21 (s, 2H), 3.22 (t, J = 12.6 Hz, 3H), 2.27 (d, J = 12.2 Hz, 2H), 1.77 (q, J = 11.8 Hz, 2H). | 625.2 |
| 160 | (E)-3-(6-(((1-(3- Cyano-4-(4- cyano-3- fluorophenyl)-5- (3-hydroxy-4- methoxyphenyl) pyridin-2- yl)piperidin-4- yl)amino)methyl) pyridazin-3-yl)-N- hydroxyacrylamide formate | 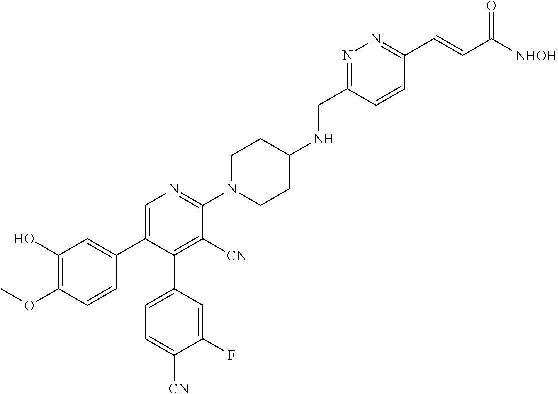 | ¹H NMR (400 MHz, DMSO-d6) δppm: 9.43 (s, 1H), 9.25 (s, 1H), 9.00 (s, 1H), 8.46 (s, 1H), 8.04 (d, J = 8.6 Hz, 1H), 7.97 (dd, J = 8.0, 6.8 Hz, 1H), 7.86 (d, J = 8.8 Hz, 1H), 7.71-7.61 (m, 2H), 7.28 (dd, J = 8.0, 1.4 Hz, 1H), 7.08 (d, J = 5.8 Hz, 1H), 6.82 (d, J = 8.2 Hz, 1H), 6.51-6.43 (m, 2H), 4.63 (s, 2H), 4.32 (d, J = 8.2 Hz, 2H), 3.72 (s, 3H), 3.60-3.40 (m, 1H), 3.15 (t, J = 12.6 Hz, 2H), 2.25 (s, 2H), 1.78 (s, 2H). | 621.2 |
| 161 | (E)-3-(4-(((1-(3- Cyano-4-(4- cyano-3- fluorophenyl)-5- (3-fluoro-5- hydroxy-4- methylphenyl) pyridin-2- yl)piperidin- 4- yl)amino)methyl) phenyl)-N- hydroxyacrylamide formate | 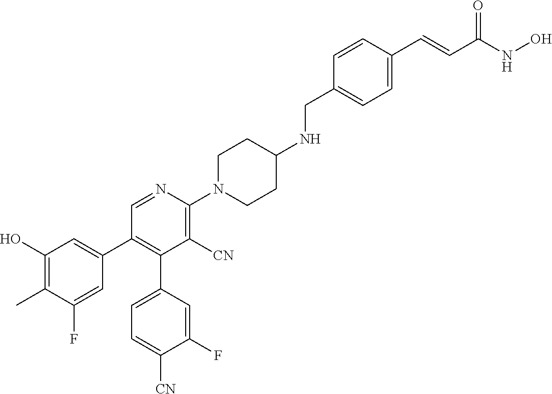 [SMILES: Cc1c(O)cc(-c2cnc(N3CCC(NCc4ccc(/C=C/C(=O)NO)cc4)CC3)c(C#N)c2-c2ccc(C#N)c(F)c2)cc1F] | ¹H NMR (400 MHz, Methanol-d4) δppm 8.52 (s, 1H), 8.35 (s, 1H), 7.67 (dd, J = 8.0, 6.6 Hz, 1H), 7.58 (d, J = 8.0 Hz, 2H), 7.51-7.43 (m, 3H), 7.31 (dd, J = 9.8, 1.6 Hz, 1H), 7.13 (dd, J = 8.0, 1.6 Hz, 1H), 6.44 (d, J = 15.8 Hz, 1H), 6.22 (dd, J = 9.8, 1.8 Hz, 1H), 6.16 (d, J = 1.4 Hz, 1H), 4.39 (d, J = 13.4 Hz, 2H), 4.23 (s, 2H), 3.51-3.32 (m, 1H), 3.15-3.00 (m, 2H), 2.25 (d, J = 12.2 Hz, 2H), 1.93 (d, J = 1.8 Hz, 3H), 1.79 (ddt, J = 21.8, 13.6, 7.4 Hz, 2H). | 621.2 |
| 162 | (E)-3-(2-Cyano-4- ((1-(3-cyano-4-(4- cyano-3- fluorophenyl)-5- (3-hydroxy-4- methoxyphenyl) pyridin-2- yl)piperidin-4- yl)amino)methyl) phenyl)-N- hydroxyacrylamide formate | 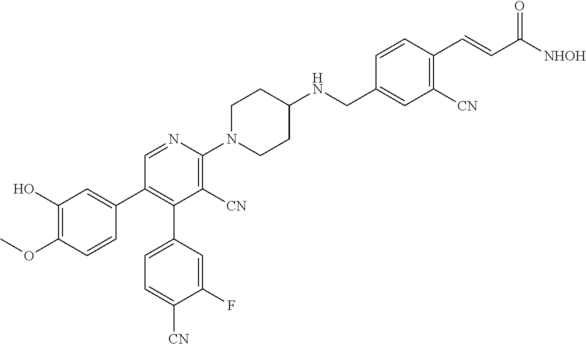 | ¹H NMR (400 MHz, DMSO-d6) δ 11.01 (s, 1H), 9.24 (s, 1H), 9.01 (s, 1H), 8.93 (s, 1H), 8.45 (d, J = 7.0 Hz, 1H), 8.05 (s, 1H), 7.97 (t, J = 7.4 Hz, 2H), 7.86 (d, J = 7.8 Hz, 1H), 7.72-7.61 (m, 2H), 7.28 (dd, J = 8.0, 1.4 Hz, 1H), 6.82 (d, J = 8.0 Hz, 1H), 6.73 (d, J = 9.6 Hz, 1H), 6.52-6.44 (m, 2H), 4.29 (d, J = 5.8 Hz, 4H), 3.72 (s, 3H), 3.45- 3.40 (m, 2H), 3.15 (t, J = 5.6 Hz, 2H), 2.22 (s, 2H), 1.71 (s, 2H). | 644.2 |
| 163 | 2-(4-(((1-(3- Cyano-4-(4- cyano-3- fluorophenyl)-5- (3-hydroxy-4- methoxyphenyl) pyridin-2- yl)piperidin-4- yl)amino)methyl) phenyl)-N- hydroxycyclopent- 1-ene-1- carboxamide formate | 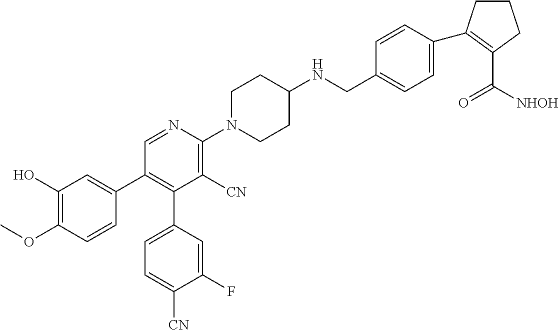 | ¹H NMR (400 MHz, DMSO-d6) δ 10.51 (s, 1H), 8.94 (s, 1H), 8.42 (s, 1H), 8.18 (s, 1H), 7.95 (dd, J = 8.0, 6.8 Hz, 1H), 7.66 (dd, J = 10.2, 1.5 Hz, 1H), 7.44-7.30 (m, 4H), 7.27 (dd, J = 8.0, 1.6 Hz, 1H), 6.80 (d, J = 8.0 Hz, 1H), 6.46 (s, 2H), 4.19 (d, J = 13.2 Hz, 2H), 3.85-3.84 (s, 2H), 3.71 (s, 3H), 3.22-3.12 (m, 2H), 2.83 (s, 1H), 2.81-2.73 (m, 2H), 2.71-2.60 (m, 2H), 2.03 (d, J = 12.4 Hz, 2H), 1.92 (p, J = 7.6 Hz, 2H), 1.50 (q, J = 11.6, 11.2 Hz, 2H). | 659.3 |
| 164 | 4-(2-[(1-(3-Cyano- 4-(4-cyano-3- fluorophenyl)-5- (3-hydroxy-4- methoxyphenyl) pyridin-2- yl)piperidin-4- yl)amino]ethoxy)- N- hydroxybenzamide formate | 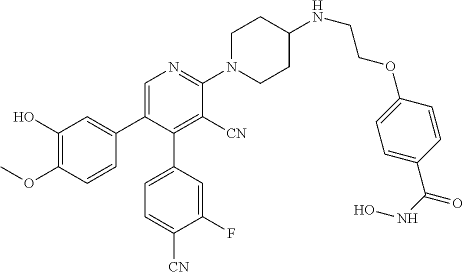 [SMILES: COc1ccc(-c2cnc(N3CCC(NCCOc4ccc(C(=O)NO)cc4)CC3)c(C#N)c2-c2ccc(C#N)c(F)c2)cc1O] | ¹H NMR (400 MHz, DMSO-d6) δ ppm: 11.07 (s, 1H), 9.00 (s, 1H), 8.90 (s, 1H), 8.43 (s, 1H), 8.12 (s, 1H), 7.96 (s, 1H), 7.74 (d, J = 8.2 Hz, 2H), 7.67 (d, J = 10.1 Hz, 1H), 7.28 (d, J = 8.1 Hz, 1H), 7.02 (d, J = 8.2 Hz, 2H), 6.81 (d, J = 7.9 Hz, 1H), 6.47 (d, J = 9.7 Hz, 2H), 4.24- 4.13 (m, 4H), 3.72 (s, 3H), 3.17 (t, J = 11.9 Hz, 4H), 3.04 (s, 1H), 2.07 (d, J = 11.8 Hz, 2H), 1.53 (d, J = 12.0 Hz, 2H). | 623.2 |
| 165 | (E)-3-(4-(((1-(3- Cyano-4-(4- cyano-3- fluorophenyl)-5- (5-hydroxy-2,4- dimethoxyphenyl) pyridin-2- yl)piperidin-4- yl)amino)methyl) phenyl-N- hydroxyacrylamide hydrochloride | 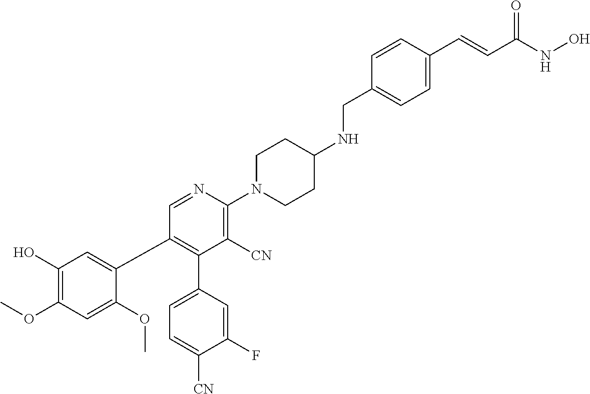 [SMILES: COc1cc(OC)c(-c2cnc(N3CCC(NCc4ccc(/C=C/C(=O)NO)cc4)CC3)c(C#N)c2-c2ccc(C#N)c(F)c2)cc1O] | ¹H NMR (400 MHz, Methanol-d4) δ ppm 8.31 (s, 1H), 7.76-7.52 (m, 6H), 7.25 (dd, J = 39.6, 9.0 Hz, 2H), 6.66-6.43 (m, 3H), 4.43 (d, J = 13.4 Hz, 2H), 4.25 (s, 2H), 3.84 (s, 3H), 3.46 (s, 3H), 3.41 (d, J = 11.8 Hz, 1H), 3.17 (t, J = 12.8 Hz, 2H), 2.30 (d, J = 12.2 Hz, 2H), 1.90- 1.77 (m, 2H). | 649.2 |
| 166 | (E)-3-(4-(((1-(3- Cyano-4-(4- cyano-3- hydroxyphenyl)-5- (3-hydroxy-4- methoxyphenyl) pyridin-2- yl)piperidin-4- yl)amino)methyl) phenyl-N- hydroxyacrylamide hydrochloride | 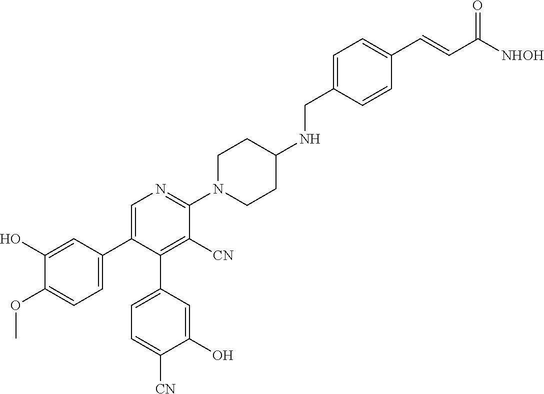 | ¹H NMR (400 MHz, Methanol-d4) δ ppm 8.40 (s, 1H), 7.65 (d, J = 7.8 Hz, 2H), 7.62-7.47 (m, 4H), 6.84- 6.77 (m, 3H), 6.60-6.44 (m, 3H), 4.40 (d, J = 13.2 Hz, 2H), 4.24 (s, 2H), 3.81 (s, 3H), 3.42-3.34 (m, 1H), 3.21-3.09 (m, 2H), 2.35- 2.24 (m, 2H), 1.83 (qd, J = 12.2, 11.4, 3.6 Hz, 2H). | 617.2 |
| 167 | 2-(4-((1-(3-Cyano- 4-(4-cyano-3- fluorophenyl)-5- (3-hydroxy-4- methoxyphenyl) pyridin-2- yl)piperidin-4- yl)amino)methyl) phenyl)-N- hydroxycyclopropane- 1- carboxamide formate | 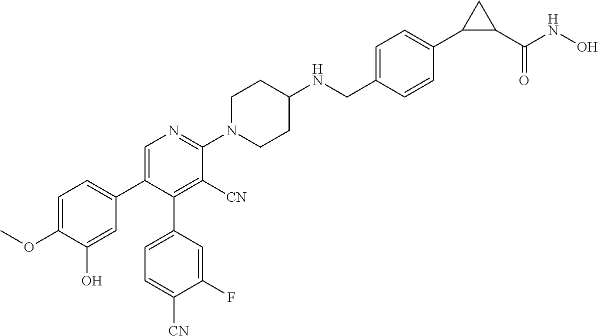 [SMILES: COc1ccc(-c2cnc(N3CCC(NCc4ccc(C5CC5C(=O)NO)cc4)CC3)c(C#N)c2-c2ccc(C#N)c(F)c2)cc1O] | ¹H NMR (400 MHz, DMSO-d6) δ 10.61 (s, 1H),9.01 (s, 1H), 8.88- 8.71 (m, 3H), 8.46 (s, 1H), 8.04- 7.93 (m, 1H), 7.67 (dd, J = 10.0, 1.4 Hz, 1H), 7.42 (d, J = 8.0 Hz, 2H), 7.28 (dd, J = 8.0, 1.4 Hz, 1H), 7.22 (d, J = 8.0 Hz, 2H), 6.82 (d, J = 8.0 Hz, 1H), 6.46 (s, 2H), 4.30 (d, J = 6.2 Hz, 2H), 4.20 (s, 2H), 3.72 (s, 3H), 3.39 (s, 1H), 3.12 (t, J = 5.6 Hz, 2H), 2.36-2.17 (m, 3H), 1.81-1.63 (m, 3H), 1.50-1.33 (m, 2H). | 633.3 |
| 168 | (E)-3-(4-(((1-(3- Cyano-4-(4- cyano-3- fluorophenyl)-5- (3-fluoro-5- hydroxy-4- methoxyphenyl) pyridin-2- yl)piperidin-4- yl)amino)methyl) phenyl)-N- hydroxyacrylamide formate | 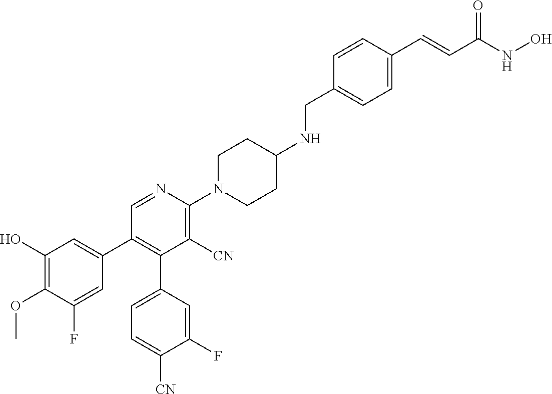 [SMILES: COc1c(O)cc(-c2cnc(N3CCC(NCc4ccc(/C=C/C(=O)NO)cc4)CC3)c(C#N)c2-c2ccc(C#N)c(F)c2)cc1F] | ¹H NMR (400 MHz, Methanol-d4) δppm 8.44 (s, 1H), 7.78 (dd, J = 8.0, 6.6 Hz, 1H), 7.67 (d, J = 7.8 Hz, 2H), 7.62-7.51 (m, 3H), 7.41 (dd, J = 9.6, 1.6 Hz, 1H), 7.25 (dd, J = 8.0, 1.6 Hz, 1H), 6.53 (d, J = 15.8 Hz, 1H), 6.45-6.29 (m, 2H), 4.58-4.44 (m, 2H), 4.32 (s, 2H), 3.83 (d, J = 1.0 Hz, 3H), 3.64- 3.42 (m, 1H), 3.29-3.09 (m, 2H), 2.48-2.14 (m, 2H), 1.86 (qd, J = 12.4, 4.1 Hz, 2H). | 637.2 |
| 169 | (E)-4-(4-(((1-(3- Cyano-4-(4- cyano-3- fluorophenyl)-5- (3-hydroxy-4- methoxyphenyl) pyridin-2- yl)piperidin-4- yl)amino)methyl) phenyl)-N- hydroxybut-3- enamide formate | 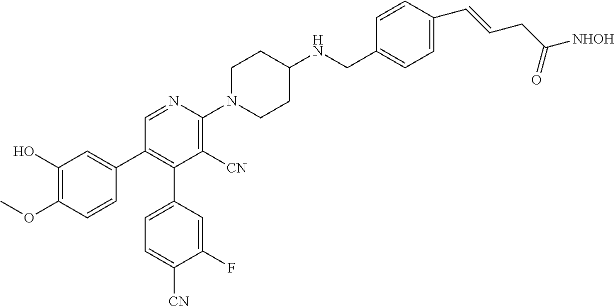 | ¹H NMR (400 MHz, DMSO) δppm: 10.50 (s, 1H), 8.99 (s, 1H), 8.77 (s, 1H), 8.52 (s, 1H), 7.95 (dd, J = 8.0, 6.8 Hz, 1H), 7.66 (dd, J = 10.0, 1.6 Hz, 1H), 7.43-7.33 (m, 4H), 7.27 (dd, J = 8.0, 1.6 Hz, 1H), 6.81 (d, J = 8.2 Hz, 1H), 6.53- 6.43 (m, 3H), 6.29 (dt, J = 15.4, 7.2 Hz, 1H), 4.21 (d, J = 13.0 Hz, 2H), 3.90 (s, 2H), 3.71 (s, 3H), 3.16 (q, J = 11.2 Hz, 2H), 2.97-2.87 (m, 2H), 2.52 (d, J = 11.4 Hz, 1H), 2.06 (d, J = 12.4 Hz, 2H), 1.53 (d, J = 12.0 Hz, 2H). | 632.3 |
| 170 | (E)-3-(4-(((1-(3- Cyano-4-(4- cyano-3- fluorophenyl)-5- (3-hydroxy-4- methoxyphenyl) pyridin-2- yl)piperidin-4- yl)amino)methyl) phenyl)-N- hydroxybut-2- enamide formate | 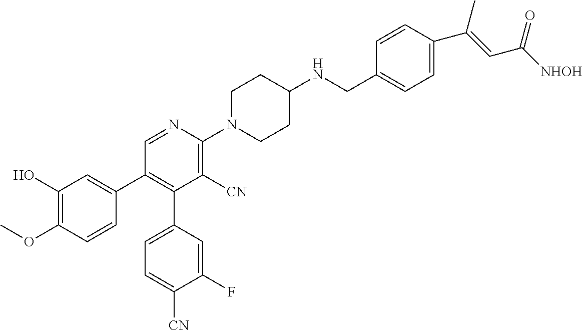 | ¹H NMR (400 MHz, DMSO-d6) δ 10.61 (s, 1H), 9.01 (s, 1H), 8.93- 8.82 (m, 2H), 8.47 (s, 1H), 7.97 (dd, J = 8.0, 6.8 Hz, 1H), 7.68 (dd, J = 10.0, 1.6 Hz, 1H), 7.62-7.52 (m, 3H), 7.52-7.42 (m, 1H), 7.28 (dd, J = 8.0, 1.6 Hz, 1H), 6.82 (d, J = 8.0 Hz, 1H), 6.47 (d, J = 8.0 Hz, 2H), 4.38-4.16 (m, 4H), 3.72 (s, 3H), 3.43 (s, 1H), 3.14 (t, J = 12.6 Hz, 2H), 2.54 (s, 3H), 2.25 (d, J = 11.2 Hz, 2H), 1.75 (d, J = 11.6 Hz, 2H). | 633.3 |
| 171 | (Z)-3-(4-(((1-(3- Cyano-4-(4- cyano-3- fluorophenyl)-5- (3-hydroxy-4- methoxyphenyl) pyridin-2- yl)piperidin-4- yl)amino)methyl) phenyl)-2-fluoro-N- hydroxyacrylamide formate | 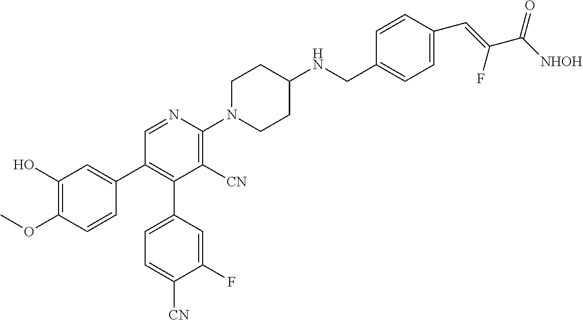 | ¹H NMR (400 MHz, Methanol-d4) δ ppm 8.44 (d, J = 1.2 Hz, 1H), 7.79-7.70 (m, 2H), 7.64 (d, J = 8.0 Hz, 1H), 7.56 (d, J = 8.0 Hz, 1H), 7.47 (d, J = 8.0 Hz, 1H), 7.37 (dd, J = 9.8, 1.6 Hz, 1H), 7.23 (dd, J = 8.1, 1.6 Hz, 1H), 6.97-6.72 (m, 2H), 6.56-6.46 (m, 2H), 4.44 (d, J = 13.4 Hz, 2H), 4.25 (s, 2H), 3.81 (s, 3H), 3.41 (d, J = 11.4 Hz, 1H), 3.22-3.12 (m, 2H), 2.30 (d, J = 12.2 Hz, 2H), 1.93-1.76 (m, 2H). | 637.2 |
| 172 | (E)-3-(4-(((1-(3- Cyano-4-(4- cyano-3- fluorophenyl)-5- (3-hydroxy-4- methoxyphenyl) pyridin-2- yl)piperidin-4- yl)amino)methyl) phenyl)-N-hydroxy- 2- methylacrylamide formate | 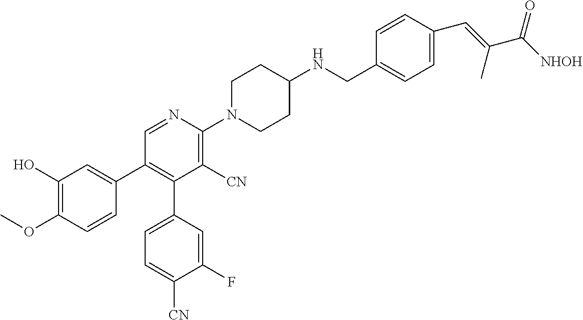 | ¹H NMR (400 MHz, Methanol-d4) δ 8.35 (d, J = 1.8 Hz, 1H), 7.65 (dd, J = 8.0, 6.6 Hz, 1H), 7.51-7.38 (m, 3H), 7.34 (d, J = 8.2 Hz, 0.5H), 7.31-7.20 (m, 1.5H), 7.18-7.07 (m, 2H), 6.72 (d, J = 8.8 Hz, 1H), 6.44-6.38 (m, 2H), 4.36 (d, J = 13.2 Hz, 2H), 4.23 (s, 2H), 3.72 (s, 3H), 3.44-3.35 (m, 1H),3.09 (t, J = 12.4 Hz, 2H), 2.25 (d, J = 11.8 Hz, 2H), 1.96 (d, J = 1.6 Hz, 2.5H), 1.78 (td, J= 12.2, 8.2 Hz, 2H), 1.50 (d, J = 7.0 Hz, 0.5H). | 633.3 |
| 173 | 5-(((1-(3-Cyano-4- (4-cyano-3- fluorophenyl)-5- (3-hydroxy-4- methoxyphenyl) pyridin-2- yl)piperidin-4- yl)amino)methyl)- N- hydroxybenzo[b] thiophene-2- carboxamide formate | 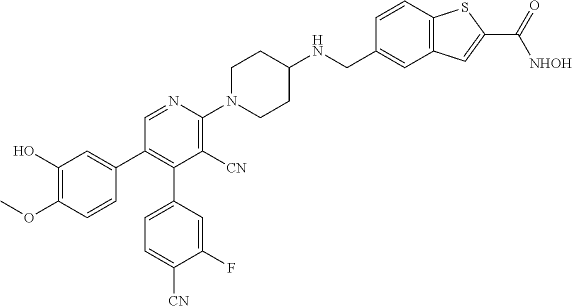 | ¹H NMR (400 MHz, Methanol-d4) δ ppm 8.52(s, 1H), 8.43 (s, 1H), 8.07-7.98 (m, 2H), 7.88 (s, 1H), 7.74 (dd, J = 8.0, 6.6 Hz, 1H), 7.56 (dd, J = 8.6, 1.6 Hz, 1H), 7.36 (dd, J = 9.8, 1.6 Hz, 1H), 7.23 (dd, J = 8.0, 1.6 Hz, 1H), 6.86-6.78 (m, 1H), 6.56-6.47 (m, 2H), 4.43 (d, J = 13.4 Hz, 2H), 4.28 (s, 2H), 3.81 (s, 3H), 3.26 (s, 1H),3.17 (t, J = 12.0 Hz, 2H), 2.28 (d, J = 12.2 Hz, 2H), 1.87-1.73 (m, 2H). | 649.2 |
| 174 | 2-((5-(((1-(3- Cyano-4-(4- cyano-3- fluorophenyl)-5- (3-hydroxy-4- methoxyphenyl) pyridin-2- yl)piperidin-4- yl)amino)methyl) pyridin-2-yl)oxy)- N- hydroxyacetamide formate | 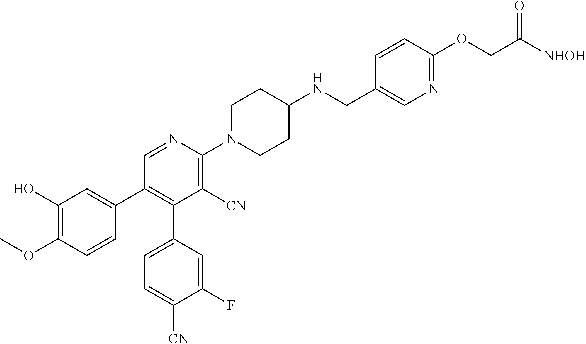 | ¹H NMR (400 MHz, Methanol-d4) δ ppm 8.47 (s, 1H), 8.42 (s, 1H), 7.74 (dd, J = 8.2, 6.6 Hz, 2H), 7.69- 7.61 (m, 1H), 7.36 (dd, J = 9.8, 1.6 Hz, 1H), 7.23 (dd, J = 8.0, 1.6 Hz, 1H), 6.88-6.77 (m, 1H), 6.60 (t, J = 9.4 Hz, 1H), 6.53-6.47 (m, 2H), 4.59 (s, 2H), 4.41 (d, J = 13.2 Hz, 2H), 3.95 (s, 2H), 3.81 (s, 3H), 3.19 (t, J = 12.8 Hz, 3H), 2.22 (d, J = 12.2 Hz, 2H), 1.86-1.67 (m, 2H). | 624.2 |

### Example 176

### Preparation of (E)-3-(4-(((1-(4-cyano-6-(4-cyano-3-fluorophenyl)-5-(3-hydroxy-4-methoxyphenyl)pyridin-2-yl)piperidin-4-yl)amino)methyl)phenyl)-N-hydroxyacrylamide formate

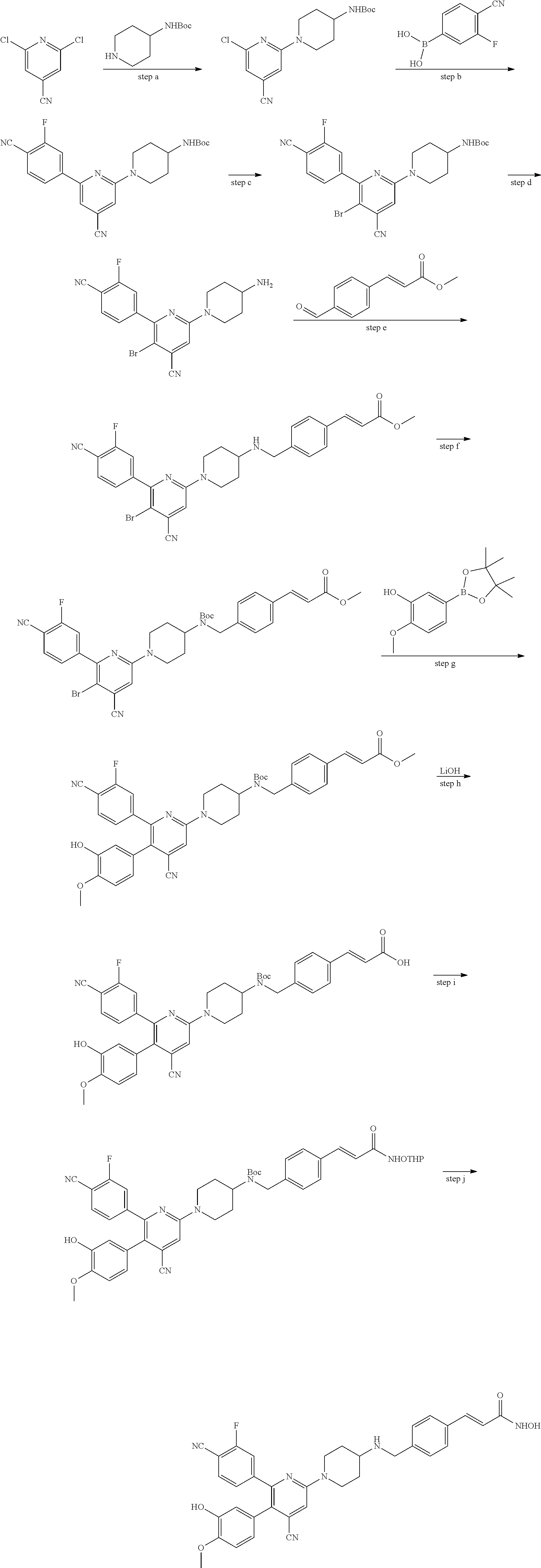

### Step a): preparation of tert-butyl (1-(6-chloro-4-cyanopyridin-2-yl)piperidin-4-yl)carbamate

2,6-Dichloroisonicotinonitrile (200 mg, 1.16 mmol) was dissolved in NMP (3 mL), and (tert-butyl piperidin-4-ylcarbamate) (348.5 mg, 1.74 mmol) was added. The reaction was stirred at 60° C. overnight, and LCMS indicated the disappearance of the starting material. The solution was added into water and extracted with ethyl acetate. The organic solution was concentrated, and the residue was mixed with silica gel, and purified by silica gel column chromatography (EA:PE=1:3), then purified by a normal-phase column to afford the product tert-butyl (1-(6-chloro-4-cyanopyridin-2-yl)piperidin-4-yl)carbamate with a yield of 74%.

ESI-MS m/z=337.1 [M+H]⁺.

### Step b): preparation of tert-butyl (1-(4-cyano-6-(4-cyano-3-fluorophenyl)pyridin-2-yl)piperidin-4-yl)carbamate

Tert-butyl (1-(6-chloro-4-cyanopyridin-2-yl)piperidin-4-yl)carbamate (240 mg, 0.71 mmol), 4-cyano-3-fluorophenylboronic acid (163.9 mg, 0.99 mmol), cesium carbonate (694 mg, 2.13 mmol), and Pd(dppf)Cl₂ (52.0 mg, 0.071 mmol) were added to a mixture of dioxane: H₂O=4:1 (4 mL). After purging with nitrogen, the mixture was heated to 115° C. with microwave and reacted for 10 minutes, and LCMS indicated the disappearance of the starting materials. The solution was concentrated, and the residue was mixed with silica gel, and purified by silica gel column chromatography (EA:PE=1:3), then further purified by a normal-phase column to afford tert-butyl (1-(4-cyano-6-(4-cyano-3-fluorophenyl)pyridin-2-yl)piperidin-4-yl)carbamate with a yield of 90.6%.

ESI-MS m/z=422.2 [M+H]⁺.

### Step c) preparation of tert-butyl (1-(5-bromo-4-cyano-6-(4-cyano-3-fluorophenyl)pyridin-2-yl)piperidin-4-yl)carbamate

Tert-butyl (1-(4-cyano-6-(4-cyano-3-fluorophenyl)pyridin-2-yl)piperidin-4-yl)carbamate (274 mg, 0.65 mmol) was dissolved in DMF (4 mL) and the mixture was cooled in an ice bath, then NBS (127.3 mg, 0.72 mmol) was added, and the reaction was stirred in an ice-water bath for 1 hour. LCMS indicated the disappearance of the starting material, then the reaction solution was poured into saturated sodium thiosulfate solution and extracted with ethyl acetate. The organic phase was washed with sodium thiosulfate solution and brine. After the solvent was removed, the residue was mixed with silica gel, and purified by silica gel column chromatography (eluent: EA:PE=1:3) to afford tert-butyl (1-(5-bromo-4-cyano-6-(4-cyano-3-fluorophenyl)pyridin-2-yl)piperidin-4-yl)carbamate with a yield of 81%.

ESI-MS m/z=500.1 [M+H]⁺.

### Step d): preparation of 6-(4-aminopiperidin-1-yl)-3-bromo-2-(4-cyano-3-fluorophenyl)isonicotinonitrile

4.0M Hydrochloric acid solution in EA (4 mL) was added to a reaction flask containing tert-butyl (1-(5-bromo-4-cyano-6-(4-cyano-3-fluorophenyl)pyridin-2-yl)piperidin-4-yl)carbamate (266 mg, 0.53 mmol), and the mixture was stirred at room temperature for 1 hour. The reaction solution was concentrated to afford 6-(4-aminopiperidin-1-yl)-3-bromo-2-(4-cyano-3-fluorophenyl)isonicotinonitrile with a yield of 80%. ESI-MS m/z=400.1 [M+H]⁺.

### Step e): preparation of methyl (E)-3-(4-(((1-(5-bromo-4-cyano-6-(4-cyano-3-fluorophenyl)pyridin-2-yl)piperidin-4-yl)amino)methyl)phenyl)acrylate

6-(4-Aminopiperidin-1-yl)-3-bromo-2-(4-cyano-3-fluorophenyl)isonicotinonitrile (170 mg, 0.42 mmol) and methyl (E)-3-(4-formylphenyl)acrylate (103.9 mg, 0.55 mmol) were added to a mixture of DCE/MeOH/CH₃COOH=20:1:0.1 (3 mL), and the solution was stirred at room temperature for 0.5 hours, then cooled in an ice bath, and sodium cyanoborohydride (445.1 mg, 2.1 mmol) was added. The reaction was slowly warmed back to room temperature and stirred for 1 hour. The reaction was monitored by LCMS until the starting materials were consumed, then the reactionsolution was poured into water and extracted with DCM. The organic phase was combined and concentrated under reduced pressure. The residue was mixed with silica gel, and purified by silica gel column chromatography (EA:PE=1:5) twice to afford methyl (E)-3-(4-(((1-(5-bromo-4-cyano-6-(4-cyano-3-fluorophenyl)pyridin-2-yl)piperidin-4-yl)amino)methyl)phenyl)acrylate with a yield of 69.2%.

ESI-MS m/z=574.1 [M+H]⁺.

### Step f): preparation of methyl (E)-3-(4-(((1-(5-bromo-4-cyano-6-(4-cyano-3-fluorophenyl)pyridin-2-yl)piperidin-4-yl) (tert-butoxycarbonyl)amino)methyl)phenyl)acrylate

Methyl (E)-3-(4-(((1-(5-bromo-4-cyano-6-(4-cyano-3-fluorophenyl)pyridin-2-yl)piperidin-4-yl)amino)methyl)phenyl)acrylate (170 mg, 0.53 mmol) was dissolved in DCM (3 mL), di-tert-butyl dicarbonate (180 mg, 0.82 mmol) and triethylamine (0.17 mL, 1.23 mmol) were added. The reaction was stirred at room temperature overnight and monitored by LCMS until the starting materials were consumed, then dilute hydrochloric acid was added to adjust the pH to 7, and the mixture was extracted with EA. The organic phases were combined and concentrated to remove the solvent. The residue was mixed with silica gel and purified by silica gel column chromatography (eluent: EA:PE=1:5) to afford methyl (E)-3-(4-(((1-(5-bromo-4-cyano-6-(4-cyano-3-fluorophenyl)pyridin-2-yl)piperidin-4-yl) (tert-butoxycarbonyl)amino)methyl)phenyl)acrylate with a yield of 74%. ESI-MS m/z: 674.2 [M+H]⁺.

### Step g) preparation of methyl (E)-3-(4-(((tert-butoxycarbonyl))(1-(4-cyano-6-(4-cyano-3-fluorophenyl)-5-(3-hydroxy-4-methoxyphenyl)pyridin-2-yl)piperidin-4-yl)amino)methyl)phenyl)acrylate

Methyl (E)-3-(4-(((1-(5-bromo-4-cyano-6-(4-cyano-3-fluorophenyl)pyridin-2-yl)piperidin-4-yl) (tert-butoxycarbonyl)amino)methyl)phenyl)acrylate (140.0 mg, 0.2 mmol), 2-methoxy-5-(tetramethyl-1,3,2-dioxaborolan-2-yl) phenol (75.1 mg, 0.3 mmol), cesium carbonate (195.5 mg, 0.6 mmol), and Pd(dppf)Cl₂ (14.6 mg, 0.02 mmol) were added to a mixture of dioxane: H₂O=4:1 (2 mL), and the reaction solution was subjected to microwave heating at 115° C. for 1.5 hours under nitrogen. The reaction was monitored by LCMS until the starting materials were consumed and the solution was concentrated. The residue was purified by silica gel column (eluent: EA:PE=1:3) to afford methyl (E)-3-(4-(((tert-butoxycarbonyl))(1-(4-cyano-6-(4-cyano-3-fluorophenyl)-5-(3-hydroxy-4-methoxyphenyl)pyridin-2-yl)piperidin-4-yl)amino)methyl)phenyl)acrylate with a yield of 74.2%. ESI-MS m/z=718.3 [M+H]⁺.

### Step h): preparation of (E)-3-(4-(((tert-butoxycarbonyl))(1-(4-cyano-6-(4-cyano-3-fluorophenyl)-5-(3-hydroxy-4-methoxyphenyl)pyridin-2-yl)piperidin-4-yl)amino)methyl)phenyl)acrylic acid

methyl (E)-3-(4-(((tert-butoxycarbonyl))(1-(4-cyano-6-(4-cyano-3-fluorophenyl)-5-(3-hydroxy-4-methoxyphenyl)pyridin-2-yl)piperidin-4-yl)amino)methyl)phenyl)acrylate (100 mg, 0.14 mmol) and lithium hydroxide (33.5 mg, 1.4 mmol) were added to a reaction solution containing THF (2.5 mL), MeOH (1.5 mL), and water (1 mL), and the mixture was stirred at room temperature for 1 hour. The pH of the reaction solution was adjusted to 4 with 2M HCl, and the mixture was extracted with ethyl acetate (5 mL×2). The organic phases were combined, washed with saturated brine (5 mL×2), dried over anhydrous sodium sulfate, filtered, and concentrated to afford (E)-3-(4-(((tert-butoxycarbonyl))(1-(4-cyano-6-(4-cyano-3-fluorophenyl)-5-(3-hydroxy-4-methoxyphenyl)pyridin-2-yl)piperidin-4-yl)amino)methyl)phenyl)acrylic acid with a yield of 71.1%. ESI-MS m/z=704.3 [M+H]⁺.

### Step i): preparation of tert-butyl (E)-(1-(4-cyano-6-(4-cyano-3-fluorophenyl)-5-(3-hydroxy-4-methoxyphenyl)pyridin-2-yl)piperidin-4-yl)(4-(3-oxo-3-(((tetrahydro-2H-pyran-2-yl)oxy)amino)prop-1-en-1-yl)benzyl)carbamate

(E)-3-(4-(((Tert-butoxycarbonyl))(1-(4-cyano-6-(4-cyano-3-fluorophenyl)-5-(3-hydroxy-4-methoxyphenyl)pyridin-2-yl)piperidin-4-yl)amino)methyl)phenyl)acrylic acid (70 mg, 0.099 mmol), O-(tetrahydro-2H-pyran-2-yl) hydroxylamine (13.9 mg, 0.12 mmol), HATU (56.5 mg, 0.15 mmol), and DIEA (38.4 mg, 0.3 mmol) were added to a reaction solution containing DMF (2 mL), and the mixture was stirred at room temperature for 1 hour. Water (5 mL) was added to quench the reaction, followed by extraction with ethyl acetate (5 mL×2). The organic phases were combined and washed with saturated brine (3 mL×2), dried with anhydrous sodium sulfate, and filtered. The residue was purified by silica gel chromatography (eluent: PE:EA=3:1) to afford tert-butyl (E)-(1-(4-cyano-6-(4-cyano-3-fluorophenyl)-5-(3-hydroxy-4-methoxyphenyl)pyridin-2-yl)piperidin-4-yl)(4-(3-oxo-3-(((tetrahydro-2H-pyran-2-yl)oxy)amino)prop-1-en-1-yl)benzyl)carbamate with a yield of 75.5%.

ESI-MS m/z=803.4 [M+H]⁺.

### Step j): preparation of (E)-3-(4-(((1-(4-cyano-6-(4-cyano-3-fluorophenyl)-5-(3-hydroxy-4-methoxyphenyl)pyridin-2-yl)piperidin-4-yl)amino)methyl)phenyl)-N-hydroxyacrylamide formate

4.0M Hydrochloric acid solution in EA (3 mL) was added to a reaction flask containing tert-butyl (E)-(1-(4-cyano-6-(4-cyano-3-fluorophenyl)-5-(3-hydroxy-4-methoxyphenyl)pyridin-2-yl)piperidin-4-yl)(4-(3-oxo-3-(((tetrahydro-2H-pyran-2-yl)oxy)amino)prop-1-en-1-yl)benzyl)carbamate (60 mg, 0.075 mmol), and the mixture was stirred at room temperature for 1 hour. After the solvent was removed, the residue was purified by Prep-HPLC (separation method 3) to afford (E)-3-(4-(((1-(4-cyano-6-(4-cyano-3-fluorophenyl)-5-(3-hydroxy-4-methoxyphenyl)pyridin-2-yl)piperidin-4-yl)amino)methyl)phenyl)-N-hydroxyacrylamide formate with a yield of 23.7%.

¹H NMR (400 MHZ, DMSO-d₆) δ ppm: 10.78 (s, 1H), 9.13 (s, 1H), 9.05 (s, 1H), 7.81 (t, J=7.4 Hz, 1H), 7.61 (s, 2H), 7.57 (d, J=13.4 Hz, 3H), 7.47 (d, J=15.8 Hz, 1H), 7.40 (dd, J=10.6, 1.4 Hz, 1H), 7.22 (dd, J=8.2, 1.6 Hz, 1H), 6.91 (d, J=8.4 Hz, 1H), 6.65-6.55 (m, 2H), 6.49 (d, J=15.8 Hz, 1H), 4.49 (s, 2H), 4.14 (s, 2H), 3.78 (s, 3H), 3.00 (t, J=12.6 Hz, 2H), 2.53 (d, J=10.8 Hz, 1H), 2.14 (s, 2H), 1.56 (s, 2H).

ESI-MS m/z=619.2 [M+H]⁺.

### Example 177

N¹-(1-(3-Cyano-4-(4-cyano-3-fluorophenyl)-5-(3-hydroxy-4-methoxyphenyl)pyridin-2-yl)piperidin-4-yl)-N⁸-hydroxyoctanediamide formate was prepared similarly according to the synthetic method of Example 58, and the structure and characterization data are as follows:

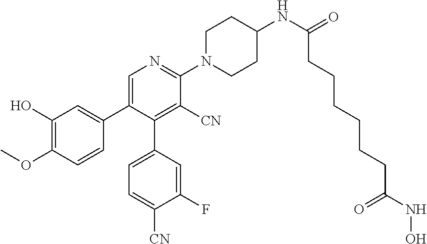 [SMILES: COc1ccc(-c2cnc(N3CCC(NC(=O)CCCCCCC(=O)NO)CC3)c(C#N)c2-c2ccc(C#N)c(F)c2)cc1O]

¹H NMR (400 MHZ, MeOD) δ ppm: 8.40 (d, J=2.2 Hz, 1H), 7.73 (t, J=7.4 Hz, 1H), 7.36 (d, J=9.8 Hz, 1H), 7.23 (dd, J=8.0, 1.4 Hz, 1H), 6.86-6.78 (m, 1H), 6.54-6.47 (m, 2H), 4.29 (d, J=13.2 Hz, 2H), 4.02-3.88 (m, 1H), 3.81 (s, 3H), 3.23 (t, J=12.0 Hz, 2H), 2.19 (t, J=7.4 Hz, 2H), 2.08 (t, J=7.4 Hz, 2H), 2.04-1.94 (m, 2H), 1.65-1.59 (m, 6H), 1.39-1.30 (m, 4H).

ESI-MS m/z=615.3 [M+H]⁺.

Examples 178-230 were prepared according to the synthetic method of Example 37 (the separation method for the compounds: hydrochloride and formate were prepared according to separation method 1 and 3, respectively), and the structure and characterization data are as follows.

| Example | Chemical name | Structure |
|---|---|---|
| 178 | (E)-3-(5- (((1-(3-Cyano- 4-(4-cyano-3- fluorophenyl)-5-(3- hydroxy-4- methoxyphenyl) pyridin- 2-yl)piperidin-4- yl)amino)methyl) furan- 2-yl)-N- hydroxyacrylamide formate | 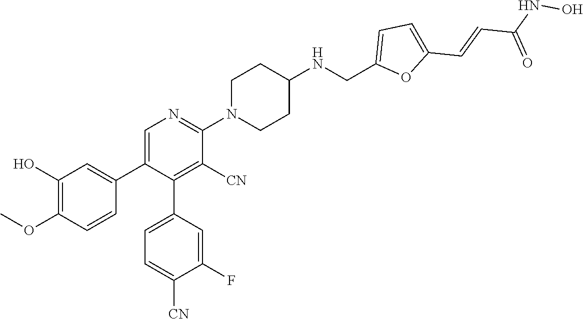 [SMILES: COc1ccc(-c2cnc(N3CCC(NCc4ccc(/C=C/C(=O)NO)o4)CC3)c(C#N)c2-c2ccc(C#N)c(F)c2)cc1O] |
| 179 | (E)-3-(4-(2-((1-(3- Cyano-4-(4-cyano-3- fluorophenyl)-5-(3- hydroxy-4- methoxyphenyl) pyridin- 2-yl)piperidin-4- yl)amino)propan-2- yl)phenyl)-N- hydroxyacrylamide formate | 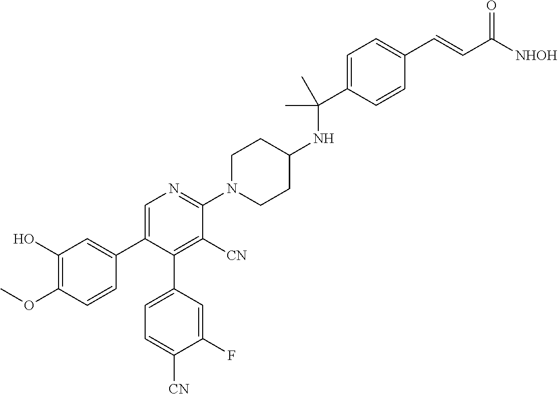 |
| 180 | (E)-3-(4- (((1-(3-Cyano- 4-(4-cyano-3- fluorophenyl)-5-(3- hydroxy-4- methoxyphenyl) pyridin- 2-yl)piperidin-4- yl)amino)methyl)-1H- indazol-7-yl)-N- hydroxyacrylamide formate | 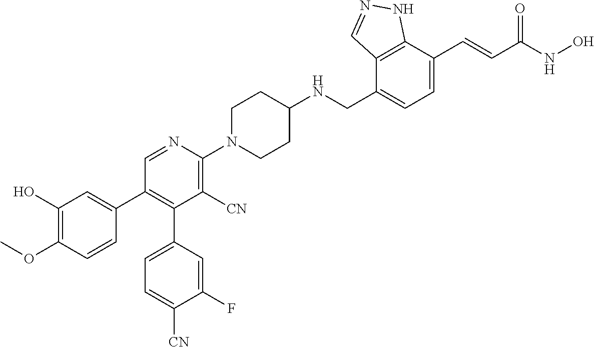 [SMILES: COc1ccc(-c2cnc(N3CCC(NCc4ccc(/C=C/C(=O)NO)c5[nH]ncc45)CC3)c(C#N)c2-c2ccc(C#N)c(F)c2)cc1O] |
| 181 | (E)-3-(2- cyano-4-((1- (3-cyano- 4-(4-cyano-3- fluorophenyl)-5-(3- hydroxy-4- methoxyphenyl) pyridin- 2-yl)piperidin-4- yl)amino)methyl) phenoxy)-N- hydroxyacetamide formate | 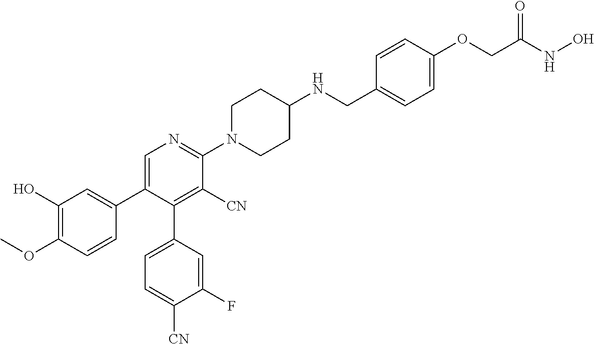 [SMILES: COc1ccc(-c2cnc(N3CCC(NCc4ccc(OCC(=O)NO)cc4)CC3)c(C#N)c2-c2ccc(C#N)c(F)c2)cc1O] |
| 182 | (E)-3-(5- (((1-(3-Cyano- 4-(4-cyano-3- fluorophenyl)-5-(1H- indazol-6-yl) pyridin-2- yl)piperidin-4- yl)amino)methyl) pyridin-2-yl)-N- hydroxyacrylamide formate | 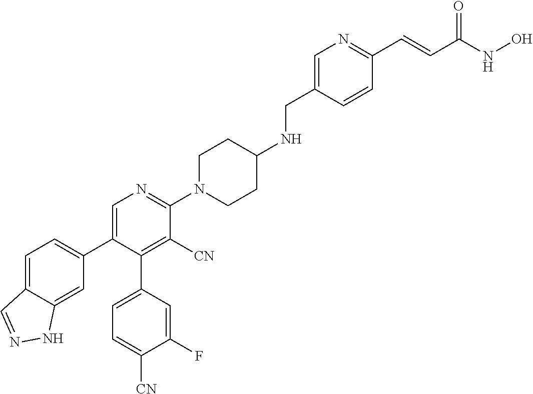 [SMILES: N#Cc1ccc(-c2c(-c3ccc4cn[nH]c4c3)cnc(N3CCC(NCc4ccc(/C=C/C(=O)NO)nc4)CC3)c2C#N)cc1F] |
| 183 | 2-(4-(((1-(3- Cyano-4- (4-cyano-3- fluorophenyl)-5-(3- hydroxy-4- methoxyphenyl) pyridin- 2-yl)piperidin-4- yl)amino)methyl) phenoxy)-N- hydroxypropanamide formate | 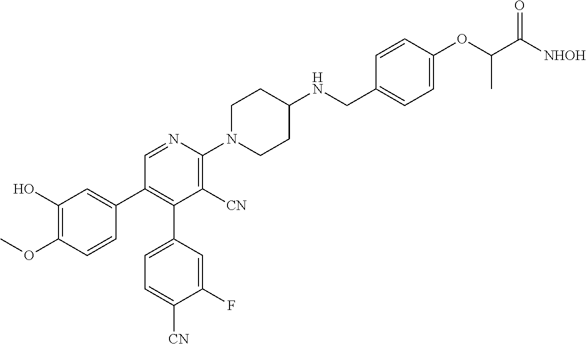 |
| 184 | (E)-3-(5- ((1-(3-Cyano- 4-(4-cyano-3- fluorophenyl)-5-(1H- indazol-6- yl)pyridin-2- yl)piperidin-4- yl)amino)methyl) pyrimidin-2-yl)-N- hydroxyacrylamide formate | 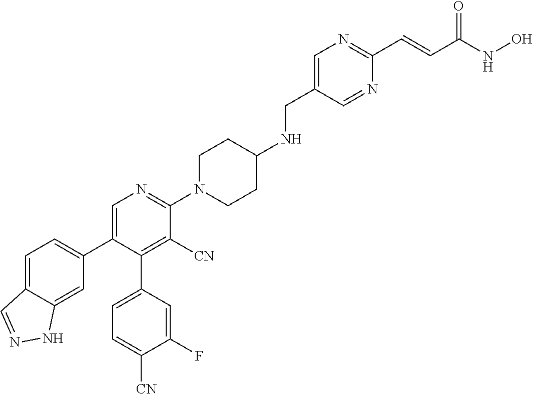 [SMILES: N#Cc1ccc(-c2c(-c3ccc4cn[nH]c4c3)cnc(N3CCC(NCc4cnc(/C=C/C(=O)NO)nc4)CC3)c2C#N)cc1F] |
| 185 | 3-(5-((1-(3- Cyano-4-(4- cyano-3- fluorophenyl)- 5-(3-hydroxy-4-(2- hydroxy-2- methylpropoxy) phenyl) pyridin-2- yl)piperidin- 4- yl)amino) methyl)thiazol- 2-yl)-N- hydroxypropanamide formate | 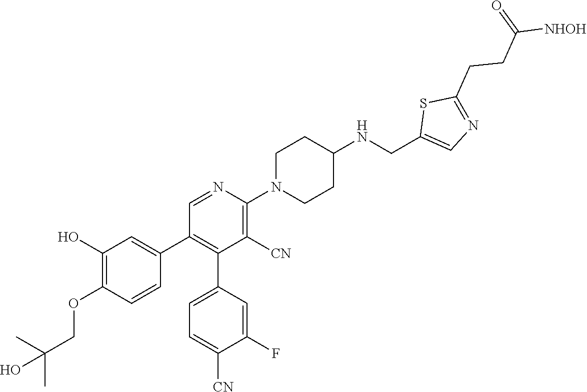 |
| 186 | (E)-3-(5- (((1-(3-Cyano- 4-(4-cyano-3- fluorophenyl)-5-(3- hydroxy-4- (trifluoromethyl) phenyl) pyridin-2-yl)piperidin- 4- yl)amino)methyl) pyrimidin-2-yl)-N- hydroxyacrylamide formate | 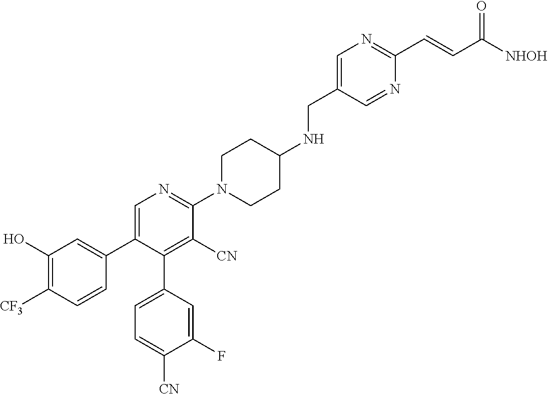 |
| 187 | (E)-3-(5- (((1-(3-Cyano- 4-(4-cyano-3- fluorophenyl)-5-(3- hydroxy- 4-(2-hydroxy- 2- methylpropoxy) phenyl) pyridin-2- yl)piperidin- 4- yl)amino)methyl) pyrimidin-2-yl)-N- hydroxyacrylamide formate | 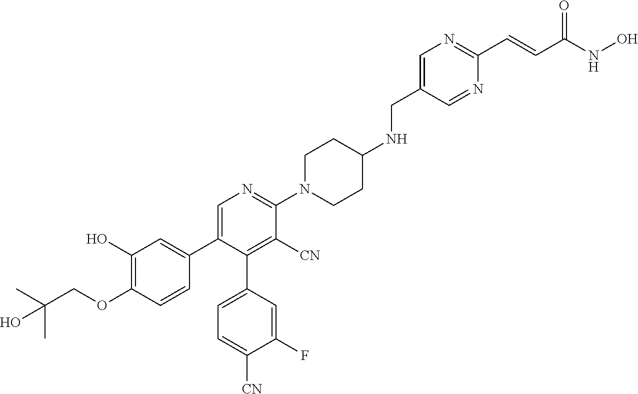 [SMILES: CC(C)(O)COc1ccc(-c2cnc(N3CCC(NCc4cnc(/C=C/C(=O)NO)nc4)CC3)c(C#N)c2-c2ccc(C#N)c(F)c2)cc1O] |
| 188 | 3-(4-(((1- (3-Cyano-4- (4-cyano-3- fluorophenyl)-5-(3- hydroxy-4- (2-hydroxy- 2- methylpropoxy) phenyl) pyridin-2- yl)piperidin- 4- yl)amino)methyl) phenyl)- N- hydroxypropanamide formate | 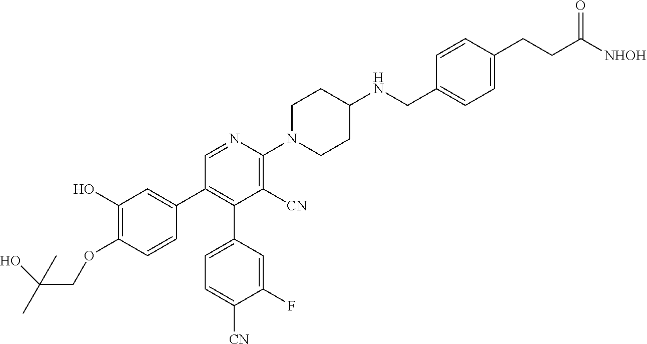 |
| 189 | (E)-3-(2- Cyano-4-((1- (3-cyano-4- (4-cyano-3- fluorophenyl)-5-(3- hydroxy-4- (2-hydroxy- 2- methylpropoxy) phenyl) pyridin-2-yl) piperidin- 4- yl)amino)methyl) phenyl)- N- hydroxyacrylamide formate | 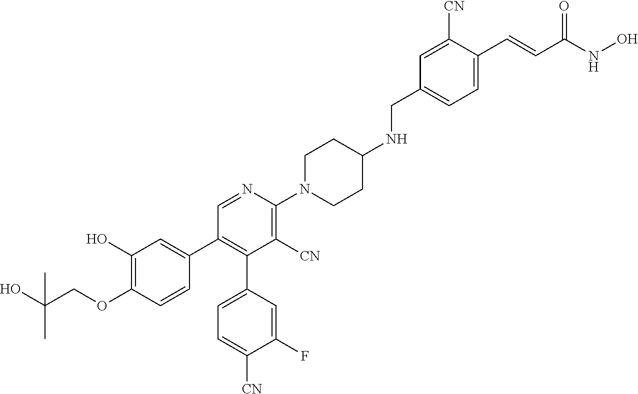 [SMILES: CC(C)(O)COc1ccc(-c2cnc(N3CCC(NCc4ccc(/C=C/C(=O)NO)c(C#N)c4)CC3)c(C#N)c2-c2ccc(C#N)c(F)c2)cc1O] |
| 190 | 3-(4-((1-(3- Cyano-4-(4- cyano-3- fluorophenyl)- 5-(3-hydroxy-4- (trifluoromethyl) phenyl) pyridin-2-yl)piperidin- 4- yl)amino)methyl) phenyl)-N- hydroxypropanamide formate | 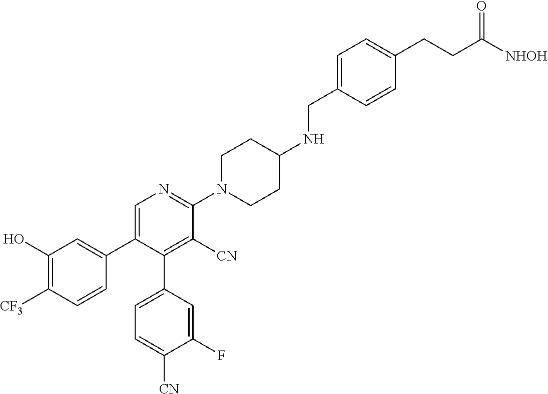 |
| 191 | 3-(5-(((1-(3- Cyano-4- (4-cyano-3- fluorophenyl)-5-(3- hydroxy-4- methoxyphenyl) pyridin- 2-yl)piperidin-4- yl)amino)methyl) pyridin-2-yl)-N- hydroxypropanamide formate | 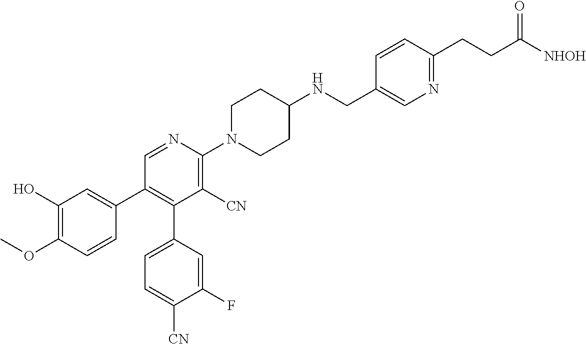 |
| 192 | 2-(4-(((1- (3-cyano-4-(4- cyano-3- fluorophenyl)- 5-(3-hydroxy-4-(2- hydroxy-2- methylpropoxy) phenyl) pyridin-2- yl)piperidin- 4- yl)amino)methyl) phenoxy)-N- hydroxyacetamide formate | 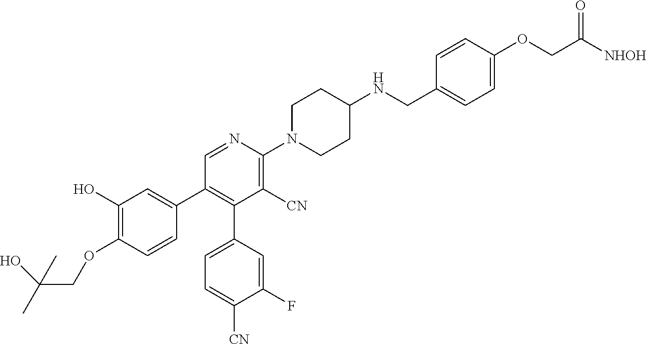 |
| 193 | 3-(2-Cyano-4-((1-(3- cyano-4-(4-cyano-3- fluorophenyl)-5-(3- hydroxy-4- methoxyphenyl) pyridin- 2-yl)piperidin-4- yl)amino)methyl) phenyl)- N- hydroxypropanamide formate | 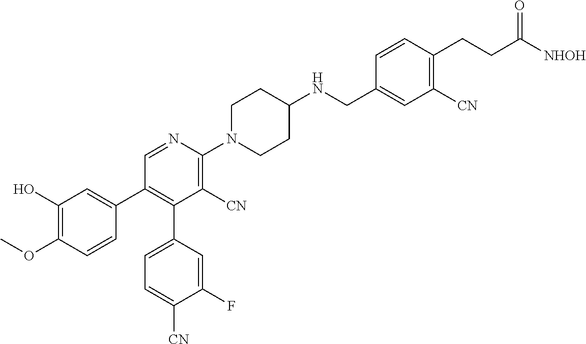 |
| 194 | 3-(6-((1-(3- Cyano-4-(4- cyano-3- fluorophenyl)- 5-(3-hydroxy-4- (methylamino) phenyl) pyridin-2- yl)piperidin-4- yl)amino)methyl) pyridin- 3-yl)-N- hydroxypropanamide hydrochloride | 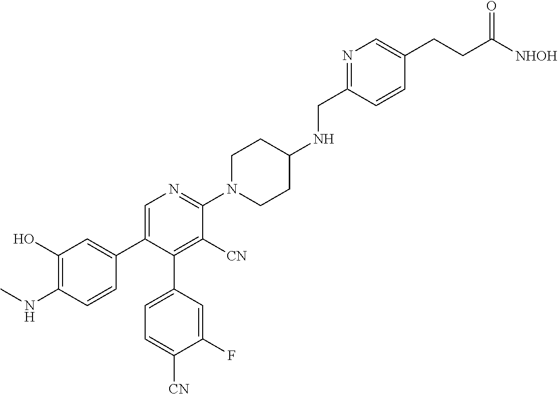 |
| 195 | 3-(5-(1-(3- Cyano-4-(4- cyano-3-fluorophenyl)- 5-(3-hydroxy-4- trifluoromethylphenyl) pyridin-2-yl) piperidin-4- ylamino)methyl) pyrimidin-2-yl)-N- hydroxypropanamide formate | 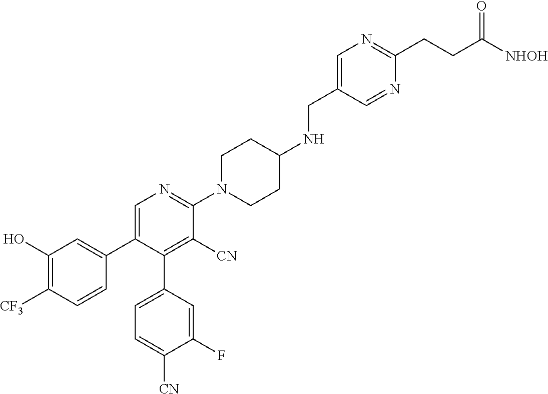 |
| 196 | (E)-3-(5- (((1-(3-Cyano- 4-(4-cyano-3- fluorophenyl)-5-(3- hydroxy-4- (methylamino)phenyl) pyridin-2-yl) piperidin-4- yl)amino)methyl) pyrimidin-2-yl)-N- hydroxyacrylamide hydrochloride | 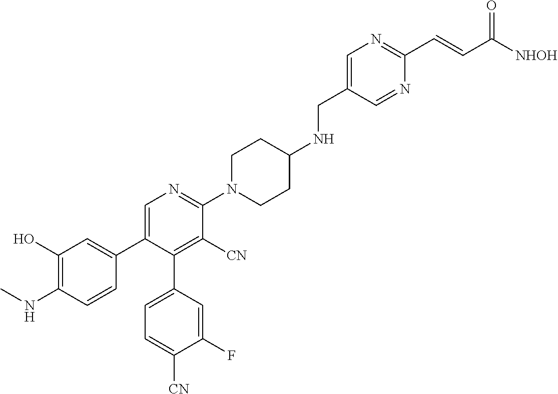 |
| 197 | (E)-3-(4- (((1-(3-Cyano- 4-(4-cyano-3- fluorophenyl)- 5-(1-(2- hydroxy-2- methylpropyl)-1H- benzo[d][1,2,3] triazol- 5-yl)pyridin-2- yl)piperidin-4- yl)amino)methyl) phenyl)- N-hydroxyacrylamide formate | 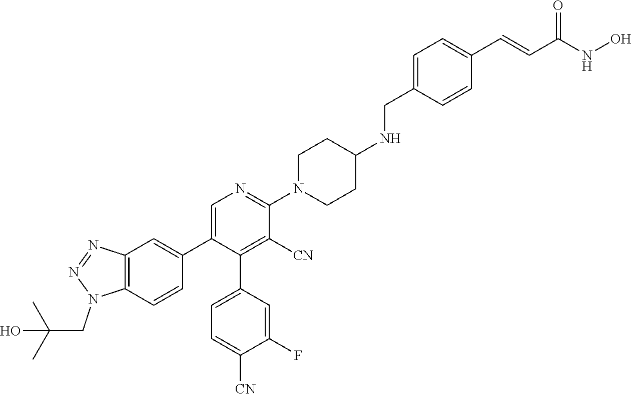 [SMILES: CC(C)(O)Cn1nnc2cc(-c3cnc(N4CCC(NCc5ccc(/C=C/C(=O)NO)cc5)CC4)c(C#N)c3-c3ccc(C#N)c(F)c3)ccc21] |
| 198 | (Z)-3-(4- (((1-(3-Cyano- 4-(4-cyano-3- fluorophenyl)-5-(3- hydroxy-4- (2-hydroxy- 2- methylpropoxy) phenyl) pyridin-2-yl)piperidin- 4- yl)amino)methyl) phenyl)- 2-fluoro-N- hydroxyacrylamide formate | 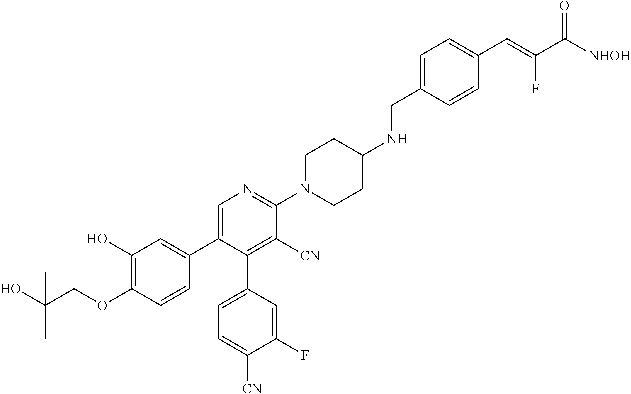 |
| 199 | (E)-3-(5- ((1-(3-Cyano- 4-(4-cyano-3- fluorophenyl)-5-(3- hydroxy-4- (2-hydroxy- 2- methylpropoxy) phenyl) pyridin-2-yl)piperidin- 4- yl)amino)methyl) pyridin-2-yl)-N- hydroxyacrylamide formate | 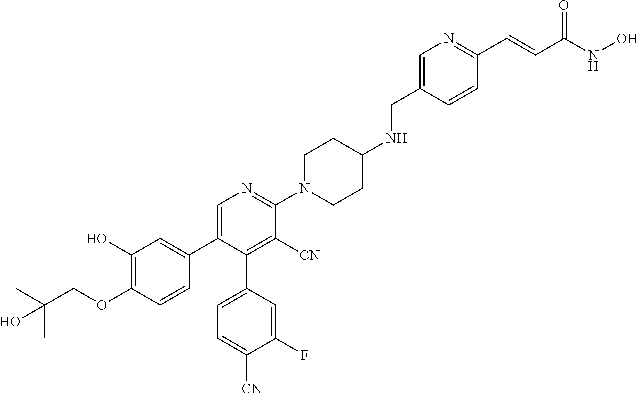 [SMILES: CC(C)(O)COc1ccc(-c2cnc(N3CCC(NCc4ccc(/C=C/C(=O)NO)nc4)CC3)c(C#N)c2-c2ccc(C#N)c(F)c2)cc1O] |
| 200 | (E)-3-(4- (((1-(3-Cyano- 4-(4-cyano-3- fluorophenyl)-5-(3- hydroxy-4- methoxyphenyl) pyridin- 2-yl)piperidin-4- yl)amino)methyl)-2- fluorophenyl)-N- hydroxyacrylamide formate | 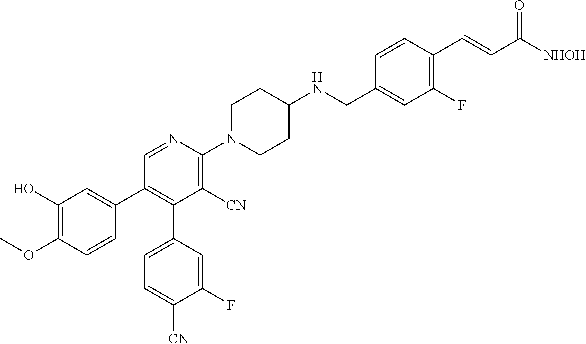 |
| 201 | 3-(4-((1-(3- Cyano-4-(4- cyano-3-fluorophenyl)- 5-(3-hydroxy-4- methoxyphenyl) pyridin- 2-yl)piperidin-4- yl)amino)methyl)-2- fluorophenyl)-N- hydroxypropanamide formate | 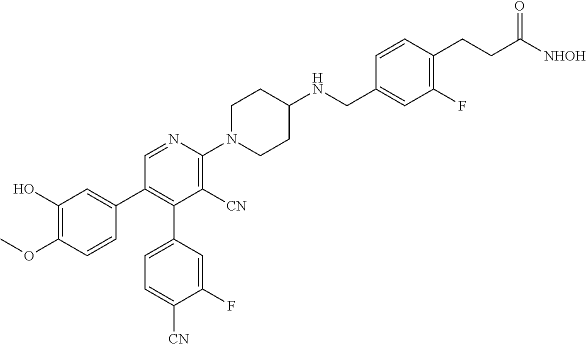 |
| 202 | (E)-3-(5-(((1-(4-(4- Cyano-3- fluorophenyl)- 5-(3-hydroxy-4- methoxyphenyl)-3- methylpyridin-2- yl)piperidin-4- yl)amino)methyl) pyrimidin-2-yl)-N- hydroxyacrylamide formate | 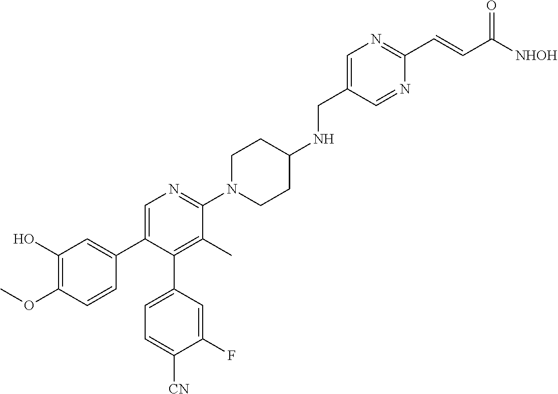 |
| 203 | (E)-3-(4- (((1-(3-Cyano- 4-(4-cyano-3- fluorophenyl)-5-(3- hydroxy-4- morpholinophenyl) pyridin- 2-yl)piperidin-4- yl)amino)methyl) phenyl- N-hydroxyacrylamide formate | 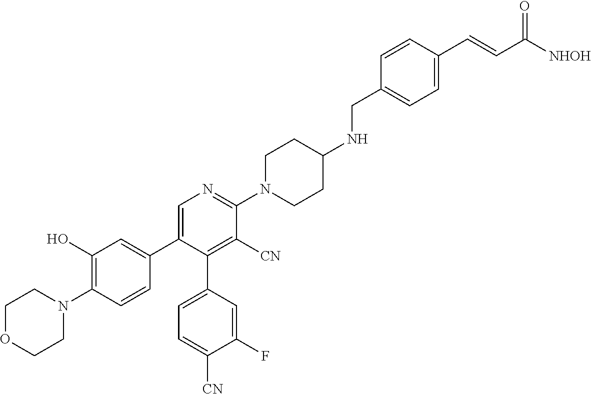 |
| 204 | 3-(5-((((1-(3-Cyano-4- (4-cyano-3- fluorophenyl)-5-(3- hydroxy-4- (2-hydroxy- 2- methylpropoxy) phenyl) pyridin-2- yl)piperidin- 4- yl)amino)methyl) pyridin-2-yl)-N- hydroxypropanamide formate | 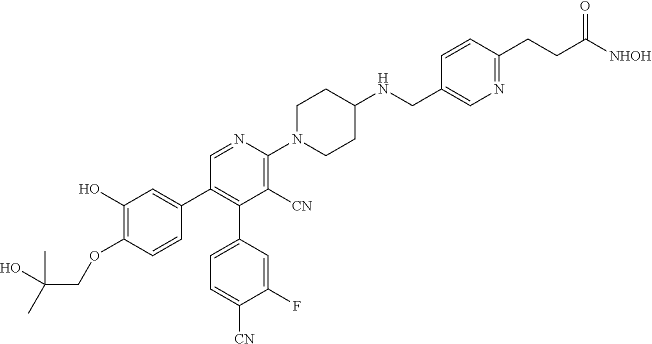 |
| 205 | (E)-3-(6- (((1-(3-Cyano- 4-(4-cyano-3- fluorophenyl)-5-(3- hydroxy-4- (2-hydroxy- 2- methylpropoxy) phenyl) pyridin-2-yl)piperidin- 4- yl)amino)methyl) pyridin-3-yl)-N- hydroxyacrylamide formate | 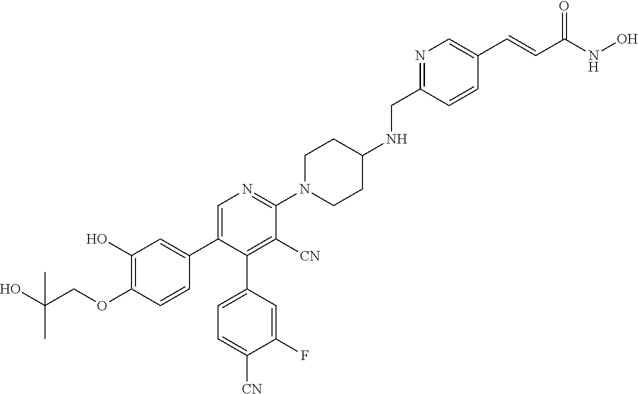 [SMILES: CC(C)(O)COc1ccc(-c2cnc(N3CCC(NCc4ccc(/C=C/C(=O)NO)cn4)CC3)c(C#N)c2-c2ccc(C#N)c(F)c2)cc1O] |
| 206 | (E)-3-(5- (((1-(3-Cyano- 4-(4-cyano-3- fluorophenyl)-5-(3- hydroxy-4- methoxyphenyl) pyridin- 2-yl)-4- methylpiperidin-4- yl)amino)methyl) pyrimidin-2-yl)-N- hydroxyacrylamide formate | 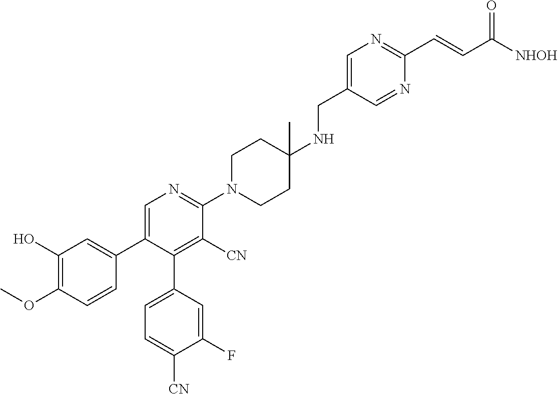 |
| 207 | (E)-3-(4-(((1-(4-(4- Cyano-3- fluorophenyl)- 5-(3-hydroxy-4-(2- hydroxy-2- methylpropoxy) phenyl)- 3-methylpyridin-2- yl)piperidin-4- yl)amino)methyl) phenyl)-N- hydroxyacrylamide hydrochloride | 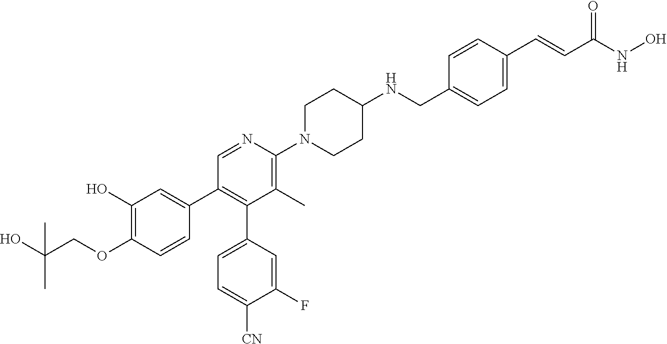 [SMILES: Cc1c(N2CCC(NCc3ccc(/C=C/C(=O)NO)cc3)CC2)ncc(-c2ccc(OCC(C)(C)O)c(O)c2)c1-c1ccc(C#N)c(F)c1] |
| 208 | (E)-3-(4-(((1-(3′,6- Dicyano-5′- (3-hydroxy- 4-methoxyphenyl)- [3,4′-bipyridin]-2′- yl)piperidin-4- yl)amino)methyl) phenyl)- N- hydroxyacrylamide | 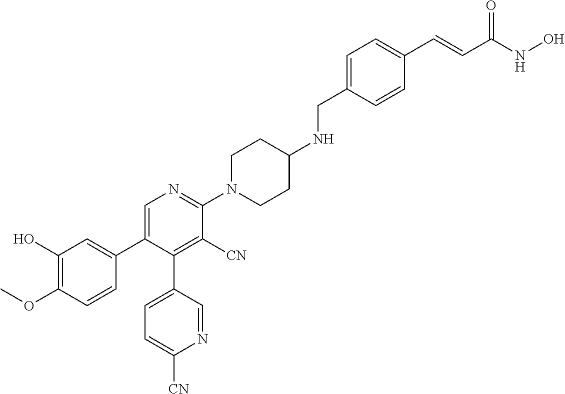 [SMILES: COc1ccc(-c2cnc(N3CCC(NCc4ccc(/C=C/C(=O)NO)cc4)CC3)c(C#N)c2-c2ccc(C#N)nc2)cc1O] |
| 209 | 3-(4-(((1- (4-(4-Cyano- 3-fluorophenyl)-5-(3- hydroxy-4- methoxyphenyl)-3- methylpyridin-2- yl)piperidin-4- yl)amino)methyl) phenyl)- N- hydroxypropanamide hydrochloride | 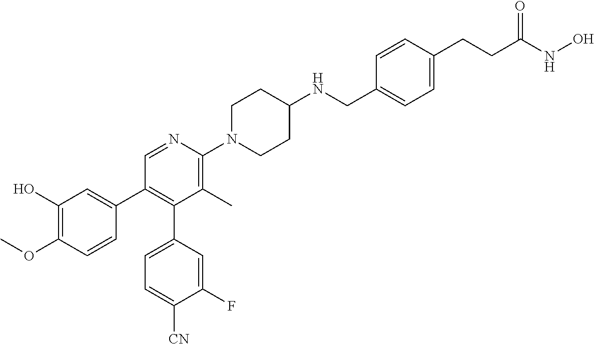 [SMILES: COc1ccc(-c2cnc(N3CCC(NCc4ccc(CCC(=O)NO)cc4)CC3)c(C)c2-c2ccc(C#N)c(F)c2)cc1O] |
| 210 | (E)-3-(4-(1-((1-(3- Cyano-4-(4-cyano-3- fluorophenyl)-5-(3- hydroxy-4- methoxyphenyl) pyridin- 2-yl)piperidin-4- yl)amino)ethyl) phenyl)- N- hydroxyacrylamide formate | 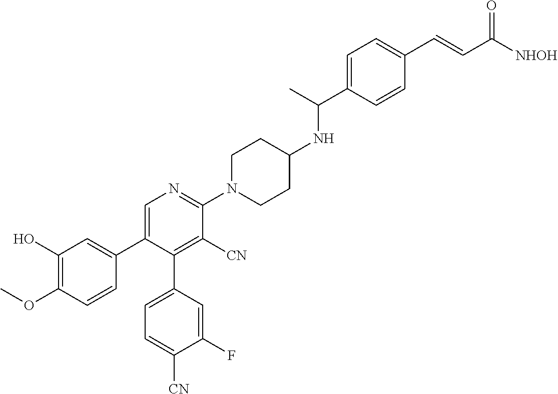 |
| 211 | (E)-3-(4- (((1-(3-Cyano- 4-(4-cyano-3- fluorophenyl)-5-(3- hydroxy-4- methoxyphenyl) pyridin- 2-yl)piperidin-4- yl)amino)methyl)-3- methylphenyl)-N- hydroxyacrylamide formate | 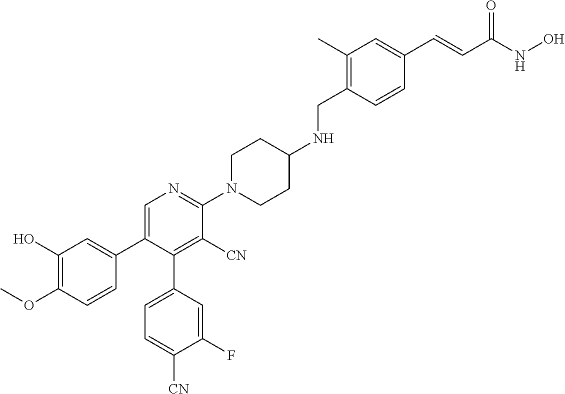 [SMILES: COc1ccc(-c2cnc(N3CCC(NCc4ccc(/C=C/C(=O)NO)cc4C)CC3)c(C#N)c2-c2ccc(C#N)c(F)c2)cc1O] |
| 212 | (E)-3-(4- (((1-(3-Cyano- 4-(4-cyano-3- fluorophenyl)-5-(3- hydroxy- 4-(2-hydroxy- 2- methylpropoxy) phenyl) pyridin-2- yl)piperidin- 4- yl)amino)methyl) phenyl)- N-hydroxybut-2- enamide hydrochloride | 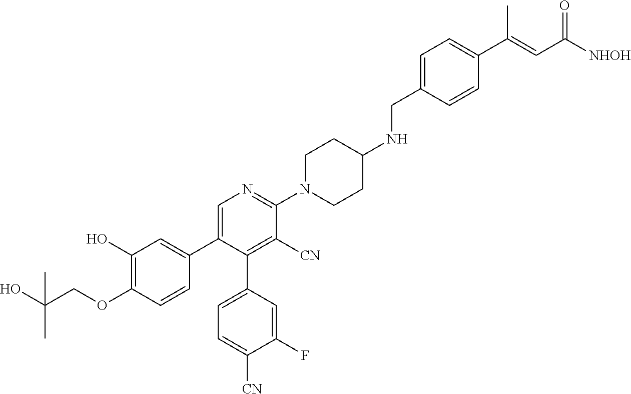 |
| 213 | (E)-3-(5-(((1- (3-Cyano- 4-(4-cyano-3- fluorophenyl)-5-(3- hydroxy-4- methoxyphenyl) pyridin- 2-yl)-4- methylpiperidin-4- yl)amino)methyl) pyridin- 2-yl)-N- hydroxyacrylamide formate | 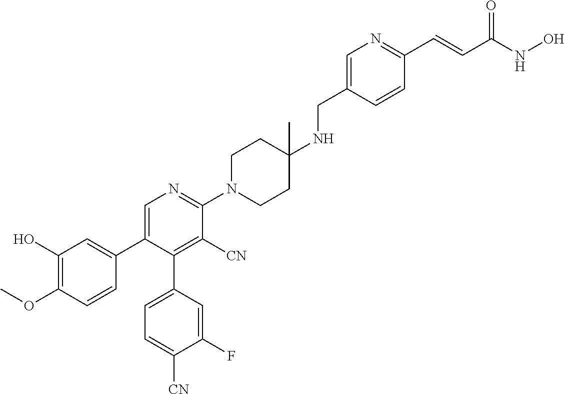 [SMILES: COc1ccc(-c2cnc(N3CCC(C)(NCc4ccc(/C=C/C(=O)NO)nc4)CC3)c(C#N)c2-c2ccc(C#N)c(F)c2)cc1O] |
| 214 | (E)-3-(4- (((1-(3-Cyano- 4-(4-cyano-3- fluorophenyl)-5-(3- hydroxy-4- (trifluoromethoxy) phenyl) pyridin-2-yl)-4- methylpiperidin-4- yl)amino)methyl) phenyl)-N- hydroxyacrylamide formate | 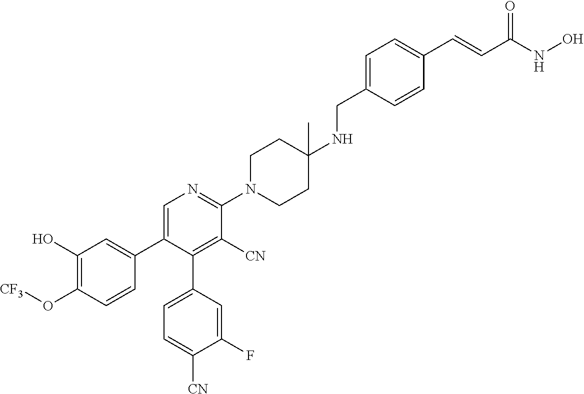 [SMILES: CC1(NCc2ccc(/C=C/C(=O)NO)cc2)CCN(c2ncc(-c3ccc(OC(F)(F)F)c(O)c3)c(-c3ccc(C#N)c(F)c3)c2C#N)CC1] |
| 215 | (E)-3-(4- (((1-(3-Cyano- 4-(4-cyano-3- fluorophenyl)-5-(3- hydroxy-4- (2-hydroxy- 2- methylpropoxy) phenyl) pyridin-2-yl)-4- methylpiperidin-4- yl)amino)methyl) phenyl)- N-hydroxyacrylamide formate | 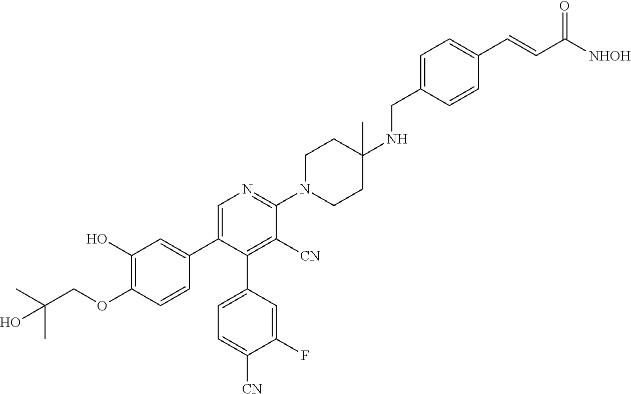 |
| 216 | 2-((4-((1-(3- cyano-4-(4- cyano-3- fluorophenyl)- 5-(3-hydroxy-4- methoxyphenyl) pyridin- 2-yl)piperidin-4- yl)amino)methyl) phenyl) thio)-N- hydroxyacetamide formate | 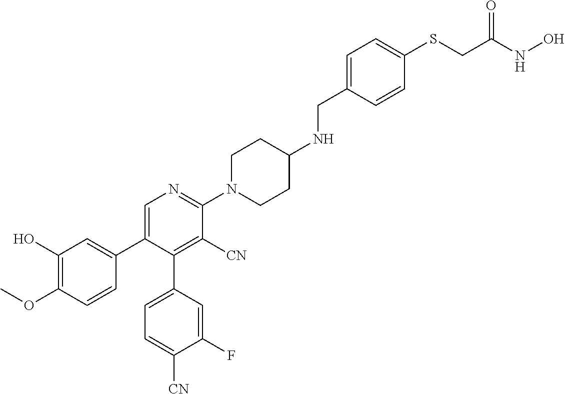 [SMILES: COc1ccc(-c2cnc(N3CCC(NCc4ccc(SCC(=O)NO)cc4)CC3)c(C#N)c2-c2ccc(C#N)c(F)c2)cc1O] |
| 217 | (E)-3-(5-(((1-(4-(4- cyano-3-fluorophenyl)- 3-(cyanomethyl)-5-(3- hydroxy-4- methoxyphenyl) pyridin- 2-yl)piperidin-4- yl)amino)methyl) pyridin- 2-yl)-N- hydroxyacrylamide formate | 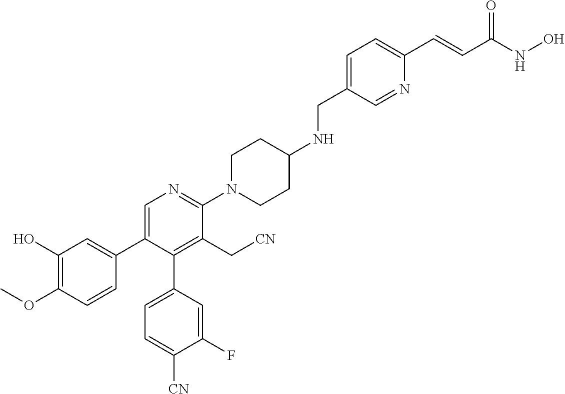 [SMILES: COc1ccc(-c2cnc(N3CCC(NCc4ccc(/C=C/C(=O)NO)nc4)CC3)c(CC#N)c2-c2ccc(C#N)c(F)c2)cc1O] |
| 218 | (E)-3-(4-(((1-(4-(4- Cyano-3- fluorophenyl)- 3-(cyanomethyl)- 5-(3- hydroxy- 4-(2-hydroxy- 2- methylpropoxy) phenyl) pyridin-2- yl)piperidin- 4- yl)amino)methyl) phenyl)- N- hydroxyacrylamide formate | 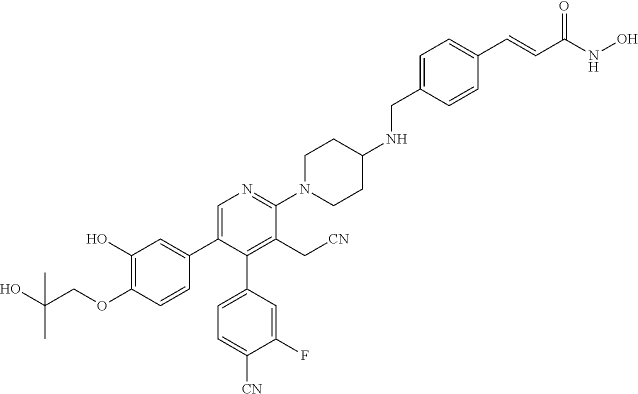 [SMILES: CC(C)(O)COc1ccc(-c2cnc(N3CCC(NCc4ccc(/C=C/C(=O)NO)cc4)CC3)c(CC#N)c2-c2ccc(C#N)c(F)c2)cc1O] |
| 219 | 3-(4-((1- (3-Cyano-4-(4- cyano-3- fluorophenyl)- 5-(3-hydroxy-4- methoxyphenyl) pyridin- 2-yl)piperidin-4- yl)amino)methyl) phenyl)- 2-fluoro-N- hydroxypropanamide formate | 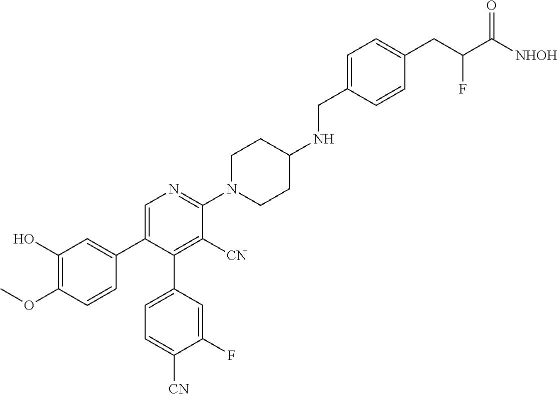 |
| 220 | (E)-3-(5- (((1-(3-Cyano- 4-(4-cyano-3- fluorophenyl)-5-(3- hydroxy-4- methoxyphenyl) pyridin- 2-yl)piperidin-4- yl)amino)methyl) pyridin- 2-yl)-N-hydroxy-2- methylacrylamide formate | 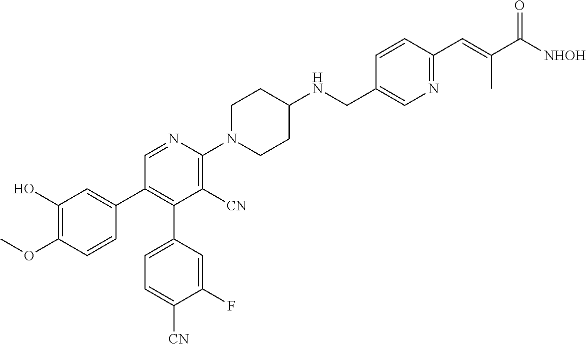 |
| 221 | (E)-3-(5-(((1-(4-(4- Cyano-3- fluorophenyl)- 3-(cyanomethyl)- 5-(3- hydroxy- 4-(2-hydroxy- 2- methylpropoxy) phenyl) pyridin-2- yl)piperidin- 4- yl)amino)methyl) pyridin- 2-yl)-N- hydroxyacrylamide formate | 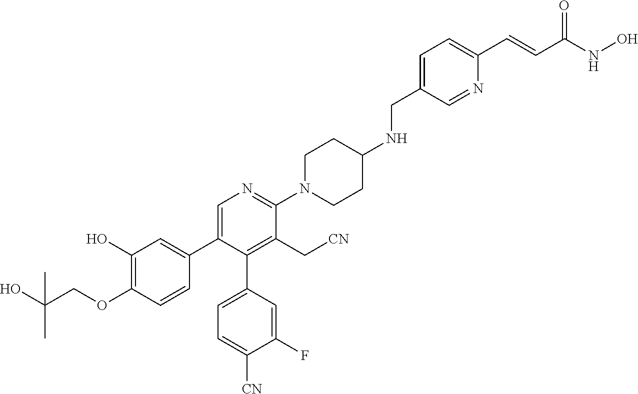 [SMILES: CC(C)(O)COc1ccc(-c2cnc(N3CCC(NCc4ccc(/C=C/C(=O)NO)nc4)CC3)c(CC#N)c2-c2ccc(C#N)c(F)c2)cc1O] |
| 222 | 3-(3-chloro-4-((1-(3- cyano-4-(4-cyano-3- fluorophenyl)-5-(3- hydroxy-4- methoxyphenyl) pyridin- 2-yl)piperidin-4- yl)amino)methyl) phenyl)- N- hydroxypropanamide formate | 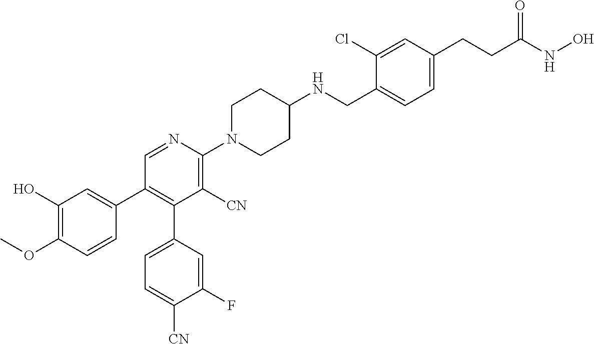 [SMILES: COc1ccc(-c2cnc(N3CCC(NCc4ccc(CCC(=O)NO)cc4Cl)CC3)c(C#N)c2-c2ccc(C#N)c(F)c2)cc1O] |
| 223 | (E)-3-(4-(2-((1-(3- Cyano-4-(4-cyano-3- fluorophenyl)-5-(3- hydroxy-4- (2-hydroxy- 2- methylpropoxy) phenyl) pyridin-2-yl) piperidin- 4-yl)amino)propan-2- yl)phenyl)-N- hydroxyacrylamide hydrochloride | 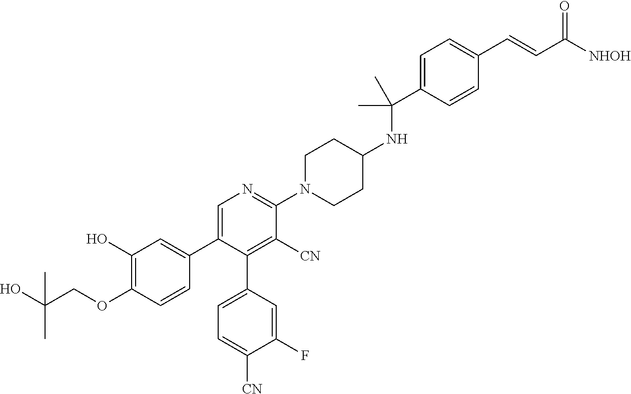 |
| 224 | (E)-3-(6- (((1-(3-Cyano- 4-(4-cyano-3- fluorophenyl)-5-(1-(2- hydroxy-2- methylpropyl)-1H- benzo[d][1,2,3]triazol- 5-yl)pyridin-2- yl)piperidin-4- yl)amino)methyl) pyridin- 3-yl)-N- hydroxyacrylamide formate | 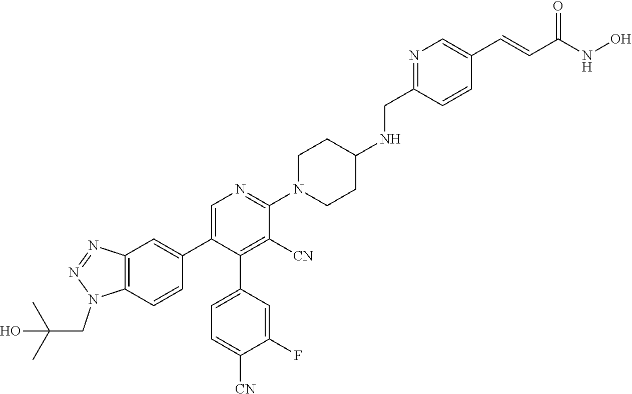 [SMILES: CC(C)(O)Cn1nnc2cc(-c3cnc(N4CCC(NCc5ccc(/C=C/C(=O)NO)cn5)CC4)c(C#N)c3-c3ccc(C#N)c(F)c3)ccc21] |
| 225 | (E)-3-(5- (((1-(3-Cyano- 4-(4-cyano-3- fluorophenyl)-5-(1-(2- hydroxy-2- methylpropyl)-1H- benzo[d][1,2,3]triazol- 5-yl)pyridin-2- yl)piperidin-4- yl)amino)methyl) pyridin- 2-)-N- hydroxyacrylamide formate | 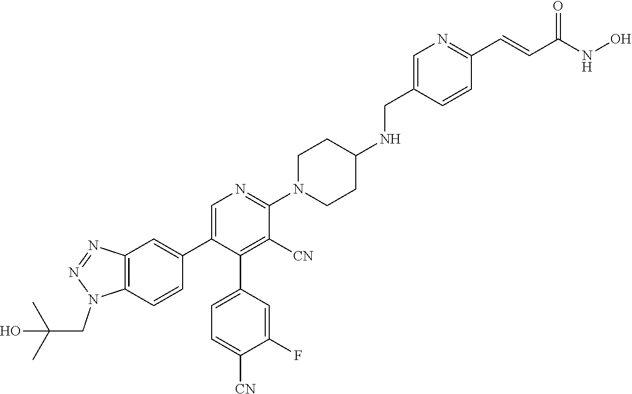 [SMILES: CC(C)(O)Cn1nnc2cc(-c3cnc(N4CCC(NCc5ccc(/C=C/C(=O)NO)nc5)CC4)c(C#N)c3-c3ccc(C#N)c(F)c3)ccc21] |
| 226 | (E)-3-(4- (((1-(3-Cyano- 4-(4-cyano-3- fluorophenyl)-5-(6,7- difluoro-1-(2-hydroxy- 2-methylpropyl)-1H- benzo[d][1,2,3]triazol- 5-yl)pyridin-2- yl)piperidin-4- yl)amino)methyl) phenyl- N-hydroxyacrylamide formate | 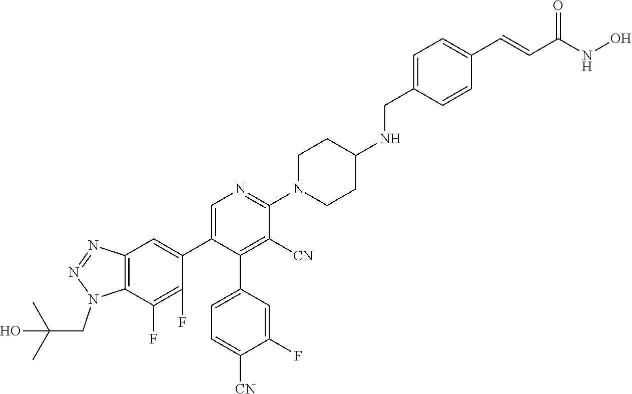 [SMILES: CC(C)(O)Cn1nnc2cc(-c3cnc(N4CCC(NCc5ccc(/C=C/C(=O)NO)cc5)CC4)c(C#N)c3-c3ccc(C#N)c(F)c3)c(F)c(F)c21] |
| 227 | (E)-3-(4- Chloro-5-(((1- (3-cyano-4- (4-cyano-3- fluorophenyl)-5-(3- hydroxy-4- methoxyphenyl) pyridin- 2-yl)piperidin-4- yl)amino)methyl) pyridin- 2-yl)-N- hydroxyacrylamide formate | 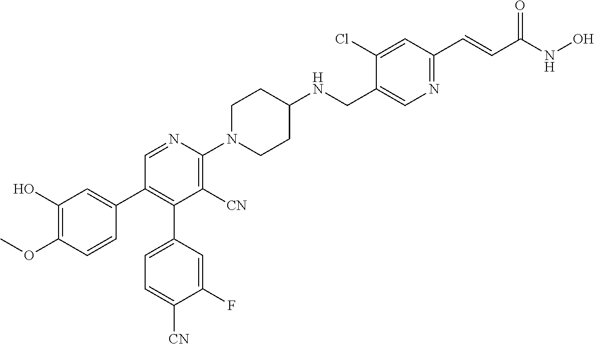 [SMILES: COc1ccc(-c2cnc(N3CCC(NCc4cnc(/C=C/C(=O)NO)cc4Cl)CC3)c(C#N)c2-c2ccc(C#N)c(F)c2)cc1O] |
| 228 | (E)-3-(4-((((1-(3- Cyano-4-(4-cyano-3- fluorophenyl)-5-(3- hydroxy-4- methoxyphenyl) pyridin- 2-yl)pyrrolidin-3- yl)methyl) amino)methyl) phenyl)-N- hydroxyacrylamide hydrochloride | 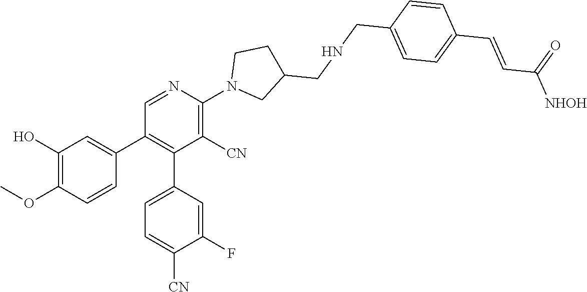 |
| 229 | (E)-3-(4- (1-(3-cyano-4- (4-cyano-3- fluorophenyl)-5-(3- hydroxy-4-((1- hydroxycyclopropyl) methoxy)phenyl) pyridin-2- yl)-4-methylpiperidin- 4- yl)amino)methyl) phenyl)- N-hydroxyacrylamide formate | 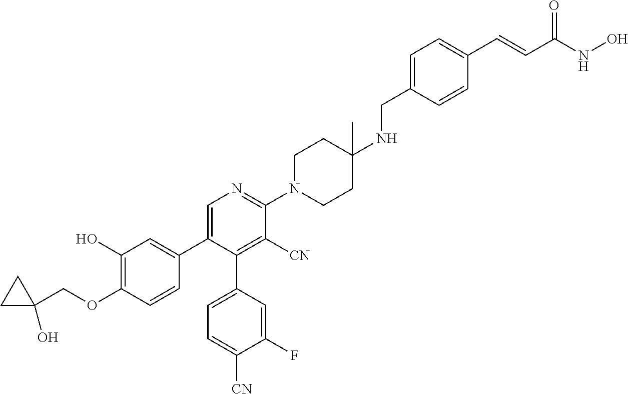 [SMILES: CC1(NCc2ccc(/C=C/C(=O)NO)cc2)CCN(c2ncc(-c3ccc(OCC4(O)CC4)c(O)c3)c(-c3ccc(C#N)c(F)c3)c2C#N)CC1] |
| 230 | (E)-3-(6- (((1-(3-Cyano- 4-(4-cyano-3- fluorophenyl)-5-(3- hydroxy-4- methoxyphenyl) pyridin- 2-yl)piperidin-4- yl)amino)methyl)-5- fluoropyridin-3-yl)-N- hydroxyacrylamide formate | 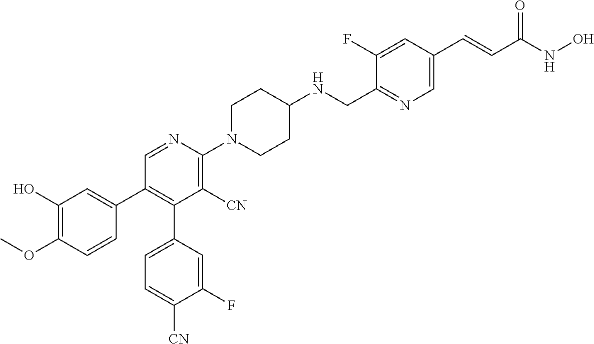 [SMILES: COc1ccc(-c2cnc(N3CCC(NCc4ncc(/C=C/C(=O)NO)cc4F)CC3)c(C#N)c2-c2ccc(C#N)c(F)c2)cc1O] |

| | | MS |
|---|---|---|
| Example | ¹H NMR | (M + H)⁺ |
| 178 | ¹H NMR (400 MHz, DMSO-d₆) δ | 609.2 |
| | 10.85 (s, 1H), 9.03 (s, 1H), 8.50 (s, | |
| | 1H), 8.47 (s, 1H), 7.98 (dd, J = 8.0, 6.8 | |
| | Hz, 1H), 7.68 (dd, J = 10.2, 1.6 Hz, | |
| | 1H), 7.37 − 7.20 (m, 2H), 6.87 − 6.72 | |
| | (m, 3H), 6.50 − 6.42 (m, 2H), 6.29 (d, | |
| | J = 5.6 Hz, 1H), 4.38 (s, 2H), 4.31 (d, | |
| | J = 8.2 Hz, 2H), 3.72 (s, 3H), 3.50 (s, | |
| | 1H), 3.13 (t, J = 6.6 Hz, 2H), 2.23 (d, | |
| | J = 5.8 Hz, 2H), 1.79 − 1.63 (m, 2H). | |
| 179 | ¹H NMR (400 MHz, DMSO-d₆) δ | 647.3 |
| | 10.78 (s, 1H), 9.03 (m, 2H), 8.39 (s, | |
| | 1H), 8.14 (s, 1H), 7.94 (dd, J = 8.0, 6.8 | |
| | Hz, 1H), 7.73 − 7.54 (m, 5H), 7.47 (d, | |
| | J = 16.0 Hz, 1H), 7.25 (dd, J = 8.0, 1.6 | |
| | Hz, 1H), 6.87 − 6.73 (m, 1H), 6.55 − | |
| | 6.40 (m, 2H), 4.09 (d, J = 13.2 Hz, | |
| | 2H), 3.71 (s, 3H), 3.44 (d, J = 7.2 Hz, | |
| | 1H), 3.00 (t, J = 12.4 Hz, 2H), 1.85 − | |
| | 1.35 (m, 10H). | |
| 180 | ¹H NMR (400 MHz, DMSO-d₆) δ | 659.3 |
| | 13.50 (s, 1H), 10.78 (s, 1H), 9.07 (s, | |
| | 1H), 9.00 (s, 1H), 8.42 (s, 1H), 8.38 (s, | |
| | 1H), 8.18 (s, 1H), 7.96 (m, 2H), 7.67 | |
| | (dd, J = 10.0, 1.6 Hz, 1H), 7.58 (d, J = | |
| | 7.4 Hz, 1H), 7.28 (dd, J = 8.0, 1.6 Hz, | |
| | 1H), 7.22 (d, J = 7.4 Hz, 1H), 6.81 (d, | |
| | J = 8.2 Hz, 1H), 6.51 − 6.41 (m, 2H), | |
| | 4.20 (m, 4H), 3.71 (s, 3H), 3.17 (t, J = | |
| | 11.8 Hz, 2H), 2.90 (s, 1H), 2.07 (d, J = | |
| | 11.8 Hz, 2H), 1.54 (d, J = 11.4 Hz, | |
| | 2H). | |
| 181 | ¹H NMR (400 MHz, DMSO-d₆) δ | 623.2 |
| | 10.85 (s, 1H), 9.01 (s, 1H), 8.97 (d, J = | |
| | 2.6 Hz, 1H), 8.46 (s, 1H), 7.97 (dd, | |
| | J = 8.0, 6.8 Hz, 1H), 7.67 (dd, J = | |
| | 10.0, 1.6 Hz, 1H), 7.43 (d, J = 8.2 Hz, | |
| | 2H), 7.28 (dd, J = 8.0, 1.4 Hz, 1H), | |
| | 7.02 (d, J = 8.2 Hz, 2H), 6.82 (d, J = | |
| | 8.0 Hz, 1H), 6.47 (d, J = 8.0 Hz, 2H), | |
| | 4.49 (s, 2H), 4.29 (d, J = 13.0 Hz, 2H), | |
| | 4.13 (s, 2H), 3.72 (s, 3H), 3.28 (s, 1H), | |
| | 3.13 (t, J = 10.4 Hz, 2H), 2.21 (d, J = | |
| | 12.0 Hz, 2H), 1.71 (s, 2H). | |
| 182 | ¹H NMR (400 MHz, Methanol-d₄) δ | 614.2 |
| | ppm 8.70 (d, J = 2.2 Hz, 1H), 8.56 (s, | |
| | 1H), 8.29 (s, 1H), 8.05 − 7.94 (m, 2H), | |
| | 7.74 − 7.58 (m, 4H), 7.44 (dd, J = 9.6, | |
| | 1.6 Hz, 1H), 7.37 − 7.31 (m, 1H), 7.23 | |
| | (dd, J = 8.0, 1.6 Hz, 1H), 6.96 (d, J = | |
| | 15.4 Hz, 1H), 6.81 (dd, J = 8.4, 1.4 Hz, | |
| | 1H), 4.51 (d, J = 13.4 Hz, 2H), 4.29 (s, | |
| | 2H), 3.40 (s, 1H), 3.22 (t, J = 12.6 Hz, | |
| | 2H), 2.32 (d, J = 12.4 Hz, 2H), 1.84 (t, | |
| | J = 11.6 Hz, 2H). | |
| 183 | ¹H NMR (400 MHz, DMSO-d₆) δ | 637.3 |
| | 10.86 (d, J = 1.8 Hz, 1H), 9.03 (s, 1H), | |
| | 8.95 (d, J = 1.7 Hz, 1H), 8.46 (s, 1H), | |
| | 7.97 (t, J = 7.4 Hz, 1H), 7.68 (dd, J = | |
| | 10.0, 1.4 Hz, 1H), 7.43 (d, J = 8.2 Hz, | |
| | 2H), 7.28 (dd, J = 8.0, 1.4 Hz, 1H), | |
| | 6.99 (d, J = 8.2 Hz, 2H), 6.82 (d, J = | |
| | 8.0 Hz, 1H), 6.47 (d, J = 8.4 Hz, 2H), | |
| | 4.71 (q, J = 6.4 Hz, 1H), 4.30 (d, J = | |
| | 13.2 Hz, 2H), 4.16 (s, 2H), 3.72 (s, | |
| | 3H), 3.35 (s, 1H), 3.12 (t, J = 12.6 Hz, | |
| | 2H), 2.23 (d, J = 11.8 Hz, 2H), 1.72 | |
| | (d, J = 12.4 Hz, 2H), 1.44 (d, J = 6.4 | |
| | Hz, 3H). | |
| 184 | ¹H NMR (400 MHz, DMSO-d₆) δ | 615.2 |
| | 13.05 (s, 1H), 11.04 (s, 1H), 9.39 − | |
| | 9.10 (m, 1H), 8.84 (s, 2H), 8.56 (s, | |
| | 1H), 8.31 (s, 1H), 8.01 (s, 1H), 7.89 | |
| | (t, J = 7.4 Hz, 1H), 7.72 (dd, J = 10.1, | |
| | 1.4 Hz, 1H), 7.60 (d, J = 8.3 Hz, 1H), | |
| | 7.43 − 7.26 (m, 3H), 7.14 (d, J = 15.5 | |
| | Hz, 1H), 6.73 (dd, J = 8.3, 1.4 Hz, | |
| | 1H), 4.25 (d, J = 13.1 Hz, 2H), 3.90 (s, | |
| | 2H), 3.22 (t, J = 11.8 Hz, 2H), 2.83 (s, | |
| | 1H), 2.05 (d, J = 12.4 Hz, 2H), 1.50 | |
| | (d, J = 12.8 Hz, 2H). | |
| 185 | ¹H NMR (400 MHz, DMSO-d₆) δ | 686.3 |
| | 10.44 (s, 1H), 9.36 (s, 1H), 8.74 (m, | |
| | 1H), 8.46 (s, 1H), 7.97 (dd, J = 8.0, 6.8 | |
| | Hz, 1H), 7.74 − 7.57 (m, 2H), 7.29 | |
| | (dd, J = 8.0, 1.6 Hz, 1H), 6.78 (d, J = | |
| | 8.4 Hz, 1H),6.56-6.38 (m, 2H), 4.66 | |
| | (m, 2H), 4.31 (d, J = 13.2 Hz, 2H), | |
| | 3.65 (s, 2H), 3.45 (s, 1H), 3.19 − 3.05 | |
| | (m, 4H), 2.34 (t, J = 7.2 Hz, 2H), 2.22 | |
| | (d, J = 12.0 Hz, 2H), 1.83 − 1.68 (m, | |
| | 2H), 1.18 (s, 6H). | |
| 186 | ¹H NMR (400 MHz, DMSO-d₆) δ | 659.2 |
| | 11.08 (s, 1H), 10.59 (s, 1H), 9.24 (s, | |
| | 1H), 8.97 (s, 2H), 8.51 (s, 1H), 7.98 | |
| | (dd, J = 8.0, 6.8 Hz, 1H), 7.76 (dd, J = | |
| | 10.0, 1.5 Hz, 1H), 7.44 − 7.34 (m, 2H), | |
| | 7.28 (dd, J = 8.0, 1.5 Hz, 1H), 7.18 (d, | |
| | J = 15.5 Hz, 1H), 6.67 (d, J = 9.5 Hz, | |
| | 2H), 4.35 (d, J = 13.0 Hz, 2H), 4.20 (s, | |
| | 2H), 3.21 (s, 2H), 2.61 (t, J = 12.5 Hz, | |
| | 1H), 1.70 (s, 2H), 1.24 (s, 2H). | |
| 187 | ¹H NMR (400 MHz, DMSO-d₆) δ | 679.3 |
| | ppm: 11.09 (s, 1H), 9.26 (s, 1H), 8.98 | |
| | (s, 2H), 8.70 (s, 1H), 8.46 (d, J = 6.4 | |
| | Hz, 1H), 7.97 (dd, J = 8.0, 6.8 Hz, 1H), | |
| | 7.67 (dd, J = 10.0, 1.5 Hz, 1H), 7.39 | |
| | (d, J = 15.5 Hz, 1H), 7.30 (dd, J = 8.0, | |
| | 1.5 Hz, 1H), 7.20 (d, J = 15.4 Hz, 1H), | |
| | 6.78 (d, J = 8.4 Hz, 1H), 6.51 (d, J = | |
| | 2.2 Hz, 1H), 6.44 (dd, J = 8.2, 2.2 Hz, | |
| | 1H), 4.70 (s, 1H), 4.32 (d, J = 12.4 Hz, | |
| | 4H), 3.65 (s, 2H), 3.17 (t, J = 12.4 Hz, | |
| | 2H), 2.61 (t, J = 12.5 Hz, 1H), 2.26 (d, | |
| | J = 12.0 Hz, 2H), 1.75 (d, J = 12.7 Hz, | |
| | 2H), 1.18 (s, 6H). | |
| 188 | ¹H NMR (400 MHz, DMSO-d₆) δ | 679.3 |
| | 10.45 (s, 1H), 8.68 (s, 1H), 8.42 (s, | |
| | 1H), 8.16 (s, 1H), 7.95 (dd, J = 8.0, 6.8 | |
| | Hz, 1H), 7.65 (dd, J = 10.0, 1.4 Hz, | |
| | 1H), 7.34 − 7.25 (m, 3H), 7.17 (d, J = | |
| | 7.8 Hz, 2H), 6.77 (d, J = 8.4 Hz, 1H), | |
| | 6.50 (d, J = 2.2 Hz, 1H), 6.43 (dd, J = | |
| | 8.2, 2.2 Hz, 1H), 4.20 (d, J = 13.0 Hz, | |
| | 2H), 3.84 (s, 2H), 3.65 (s, 2H), 3.16 (t, | |
| | J = 11.8 Hz, 2H), 2.87 (s, 1H), 2.80 (t, | |
| | J = 7.6 Hz, 2H), 2.61 (t, J = 12.5 Hz, | |
| | 1H), 2.25 (t, J = 7.6 Hz, 2H), 2.04 (d, | |
| | J = 12.0 Hz, 2H), 1.51 (q, J = 11.0, | |
| | 10.6 Hz, 2H), 1.18 (s, 6H). | |
| 189 | ¹H NMR (400 MHz, Methanol-d₄) δ | 702.3 |
| | 8.51 (s, 1H), 8.20 (s, 1H), 7.85 (d, J = | |
| | 4.2 Hz, 2H), 7.76 (t, J = 13.0 Hz, 2H), | |
| | 7.65 (dd, J = 8.0, 6.6 Hz, 1H), 7.27 | |
| | (dd, J = 9.6, 1.4 Hz, 1H), 7.14 (dd, J = | |
| | 8.0, 1.6 Hz, 1H), 6.77 − 6.59 (m, 2H), | |
| | 6.46 (d, J = 2.2 Hz, 1H), 6.39 (dd, J = | |
| | 8.2, 2.2 Hz, 1H), 4.37 (d, J = 13.3 Hz, | |
| | 2H), 4.28 (s, 2H), 3.68 (s, 2H), 3.42 (s, | |
| | 1H), 3.10 (t, J = 12.6 Hz, 2H), 2.24 (d, | |
| | J = 11.8 Hz, 2H), 1.77 (d, J = 12.4 Hz, | |
| | 2H), 1.20 (s, 6H). | |
| 190 | ¹H NMR (400 MHz, DMSO-d₆) δ | 659.2 |
| | 10.57 (s, 1H), 10.35 (s, 1H), 8.68 (s, | |
| | 1H), 8.52 (s, 1H), 8.48 (d, J = 2.0 Hz, | |
| | 1H), 8.14 (s, 1H), 7.97 (t, J = 7.5 Hz, | |
| | 1H), 7.75 (d, J = 9.9 Hz, 1H), 7.40 (d, | |
| | J = 8.3 Hz, 1H), 7.31 (d, J = 7.6 Hz, | |
| | 1H), 7.27 (d, J = 8.2 Hz, 1H), 7.17 (s, | |
| | 1H), 6.67 (s, 2H), 4.27 (d, J = 13.1 Hz, | |
| | 2H), 3.87 (s, 2H), 3.19 (d, J = 12.4 Hz, | |
| | 2H), 2.80 (t, J = 7.6 Hz, 2H), 2.33 (s, | |
| | 1H), 2.25 (t, J = 7.7 Hz, 2H), 2.06 (d, | |
| | J = 12.5 Hz, 2H), 1.53 (d, J = 11.9 Hz, | |
| | 2H). | |
| 191 | ¹H NMR (400 MHz, DMSO-d₆) δ | 622.3 |
| | ppm: 10.39 (s, 1H), 8.99 (s, 1H), 8.67 | |
| | (s, 1H), 8.52 (s, 1H), 8.44 (s, 1H), 7.96 | |
| | (t, J = 7.4 Hz, 1H), 7.76 (s, 1H), 7.66 | |
| | (d, J = 10.1 Hz, 1H), 7.28 (d, J = 8.0 | |
| | Hz, 2H), 6.81 (d, J = 8.1 Hz, 1H), 6.47 | |
| | (d, J = 9.0 Hz, 2H), 4.24 (d, J = 13.1 | |
| | Hz, 2H), 4.00 (s, 2H), 3.76 − 3.63 (m, | |
| | 3H), 3.16 (t, J = 12.3 Hz, 3H), 2.96 (t, | |
| | J = 7.7 Hz, 2H), 2.39 (t, J = 7.7 Hz, | |
| | 2H), 2.11 (s, 2H), 1.59 (s, 2H). | |
| 192 | ¹H NMR (400 MHz, DMSO-d₆) δ | 681.3 |
| | ppm: 10.83 (s, 1H), 9.04 − 8.89 (m, | |
| | 1H), 8.70 (s, 1H), 8.45 (s, 1H), 7.97 | |
| | (dd, J = 8.0, 6.8 Hz, 1H), 7.67 (dd, J = | |
| | 10.1, 1.5 Hz, 1H), 7.41 (d, J = 8.3 Hz, | |
| | 2H), 7.29 (dd, J = 8.0, 1.5 Hz, 1H), | |
| | 7.00 (d, J = 8.6 Hz, 2H), 6.78 (d, J = 8.4 | |
| | Hz, 1H), 6.50 (d, J = 2.2 Hz, 1H), 6.43 | |
| | (dd, J = 8.3, 2.2 Hz, 1H), 4.71 (s, 1H), | |
| | 4.48 (s, 2H), 4.28 (d, J = 13.3 Hz, 2H), | |
| | 4.07 (s, 2H), 3.65 (s, 2H), 3.13 (t, J = | |
| | 12.9 Hz, 2H), 3.13 (t, J = 12.9 Hz, | |
| | 2H), 2.65 (d, J = 12.0 Hz, 1H), 1.66 (d, | |
| | J = 12.1 Hz, 2H), 1.18 (s, 6H). | |
| 193 | ¹H NMR (400 MHz, DMSO-d₆) δ | 646.2 |
| | 10.40 (s, 1H), 8.99 (s, 1H), 8.41 (s, | |
| | 1H), 8.16 (s, 1H), 7.95 (dd, J = 8.0, 6.8 | |
| | Hz, 1H), 7.76 (d, J = 1.8 Hz, 1H), 7.64 | |
| | (ddd, J = 13.6, 9.0, 1.6 Hz, 2H), 7.39 | |
| | (d, J = 8.0 Hz, 1H), 7.27 (dd, J = 8.0, | |
| | 1.6 Hz, 1H), 6.80 (d, J = 8.0 Hz, 1H), | |
| | 6.51 − 6.42 (m, 2H), 4.16 (d, J = 13.0 | |
| | Hz, 2H), 3.80 (s, 2H), 3.71 (s, 3H), | |
| | 3.17 (t, J = 12.0 Hz, 2H), 3.00 (t, J = | |
| | 7.6 Hz, 2H), 2.70 (s, 1H), 2.34 (t, J = | |
| | 7.6 Hz, 2H), 1.98 (d, J = 12.6 Hz, 2H), | |
| | 1.44 (d, J = 11.6 Hz, 2H). | |
| 194 | ¹H NMR (400 MHz, DMSO-d₆) δ | 621.3 |
| | 10.79 (s, 1H), 10.44 (s, 1H), 9.52 (s, | |
| | 2H), 8.54 (s, 1H), 8.48 (s, 1H), 7.97 | |
| | (dd, J = 8.0, 6.8 Hz, 1H), 7.79 (d, J = | |
| | 8.0 Hz, 1H), 7.71 (d, J = 10.0 Hz, 1H), | |
| | 7.57 (d, J = 9.2 Hz, 1H), 7.32 − 7.21 | |
| | (m, 2H), 6.65 (d, J = 8.0 Hz, 2H), 4.36 | |
| | (d, J = 8.8 Hz, 4H), 3.44 (s, 1H), 3.14 | |
| | (t, J = 12.8 Hz, 2H), 2.94 − 2.85 (m, | |
| | 2H), 2.79 (d, J = 1.6 Hz, 3H), 2.38 − | |
| | 2.23 (m, 4H), 1.83 (d, J = 12.4 Hz, | |
| | 2H). | |
| 195 | ¹H NMR (400 MHz, Methanol-d₄) 8 | 661.2 |
| | 8.78 (s, 2H), 8.54 (s, 1H), 8.45 (s, 1H), | |
| | 7.75 (dd, J = 8.0, 6.6 Hz, 1H), 7.43 | |
| | (dd, J = 9.7, 1.5 Hz, 1H), 7.37 (d, J = | |
| | 8.0 Hz, 1H), 7.20 (dd, J = 8.0, 1.5 Hz, | |
| | 1H), 6.64 (d, J = 9.9 Hz, 2H), 4.47 (d, | |
| | J = 13.5 Hz, 2H), 4.12 (s, 2H), 3.26 (d, | |
| | J = 7.4 Hz, 2H), 3.24 (s, 1H), 3.19 (d, | |
| | J = 12.8 Hz, 2H), 2.64 (t, J = 7.4 Hz, | |
| | 2H), 2.23 (d, J = 12.3 Hz, 2H), 1.72 | |
| | (q, J = 11.5 Hz, 2H). | |
| 196 | ¹H NMR (400 MHz, DMSO-d₆) δ | 620.3 |
| | 10.80 (s, 1H), 10.44 (s, 1H), 9.87 (s, | |
| | 2H), 9.12 − 9.07 (m, 2H), 8.49 (s, 1H), | |
| | 8.00 − 7.93 (m, 1H), 7.72 (dd, J = | |
| | 10.0, 1.6 Hz, 1H), 7.42 − 7.17 (m, 4H), | |
| | 6.67 (d, J = 7.2 Hz, 2H), 4.39 − 4.30 | |
| | (m, 4H), 4.18 − 4.16 (m, 1H), 3.45 (d, | |
| | J = 7.2 Hz, 1H), 3.18 (t, J = 12.4 Hz, | |
| | 2H), 2.80 (s, 3H), 2.31 (m, 2H), 1.85 | |
| | (m, 2H). | |
| 197 | ¹H NMR (400 MHz, DMSO-d₆) δ | 686.3 |
| | 10.72 (s, 1H), 8.57 (s, 1H), 8.19 (s, | |
| | 1H), 7.91 (t, J = 7.4 Hz, 1H), 7.82 (d, | |
| | J = 1.4 Hz, 1H), 7.77 − 7.66 (m, 2H), | |
| | 7.52 (d, J = 7.8 Hz, 2H), 7.50 − 7.40 | |
| | (m, 3H), 7.34 (dd, J = 7.9, 1.5 Hz, 1H), | |
| | 7.13 (dd, J = 8.6, 1.5 Hz, 1H), 6.44 (d, | |
| | J = 5.8 Hz, 1H), 4.77 (s, 1H), 4.55 (s, | |
| | 2H), 4.24 (d, J = 13.0 Hz, 2H), 3.83 (s, | |
| | 2H), 3.22 (t, J = 12.0 Hz, 2H), 2.77 (s, | |
| | 1H), 2.01 (t, J = 8.0 Hz, 2H), 1.48 (d, | |
| | J = 11.4 Hz, 2H), 1.12 (d, J = 3.4 Hz, | |
| | 6H). | |
| 198 | ¹H NMR (400 MHz, DMSO-d₆) δ | 695.3 |
| | ppm: 9.26 (s, 1H), 8.69 (s, 1H), 8.43 | |
| | (d, J = 1.5 Hz, 1H), 8.14 (s, 1H), 7.96 | |
| | (t, J = 7.4 Hz, 1H), 7.71 − 7.59 (m, | |
| | 2H), 7.51 (dd, J = 18.6, 8.0 Hz, 2H), | |
| | 7.40 (d, J = 7.9 Hz, 1H), 7.29 (dd, J = | |
| | 8.0, 1.5 Hz, 1H), 6.77 (d, J = 8.6 Hz, | |
| | 2H), 6.50 (d, J = 2.1 Hz, 1H), 6.43 (dd, | |
| | J = 8.3, 2.2 Hz, 1H), 4.71 (s, 1H), 4.21 | |
| | (d, J = 12.7 Hz, 2H), 3.94 (s, 2H), 3.65 | |
| | (s, 2H), 3.16 (t, J = 12.0 Hz, 2H), 2.61 | |
| | (t, J = 12.5 Hz, 1H), 2.07 (s, 2H), 1.53 | |
| | (d, J = 11.8 Hz, 2H), 1.18 (s, 6H). | |
| 199 | ¹H NMR (400 MHz, DMSO-d₆) δ | 678.3 |
| | 10.88 (s, 1H), 9.09 (s, 1H), 8.68 (s, | |
| | 1H), 8.60 (d, J = 2.1 Hz, 1H), 8.42 (s, | |
| | 1H), 8.14 (s, 1H), 7.96 (dd, J = 8.0, 6.8 | |
| | Hz, 1H), 7.83 (dd, J = 7.9, 2.2 Hz, | |
| | 1H), 7.66 (dd, J = 10.1, 1.5 Hz, 1H), | |
| | 7.55 (d, J = 7.9 Hz, 1H), 7.29 (dd, J = | |
| | 8.0, 1.5 Hz, 1H), 6.91 (d, J = 15.4 Hz, | |
| | 1H), 6.77 (d, J = 8.3 Hz, 1H), 6.50 (d, | |
| | J = 2.2 Hz, 1H), 6.43 (dd, J = 8.2, 2.2 | |
| | Hz, 1H), 4.71 (s, 1H), 4.19 (d, J = 13.1 | |
| | Hz, 2H), 3.89 (s, 2H), 3.64 (s, 2H), | |
| | 3.17 (t, J = 12.1 Hz, 2H), 2.54 (s, 1H), | |
| | 2.02 (d, J = 12.3 Hz, 2H), 1.48 (q, J = | |
| | 11.2 Hz, 2H), 1.18 (s, 6H). | |
| 200 | ¹H NMR (400 MHz, DMSO-d₆) δ | 637.2 |
| | 9.10 (s, 1H), 9.00 (s, 1H), 8.51 (s, 1H), | |
| | 8.43 (s, 1H), 7.96 (t, J = 7.4 Hz, 1H), | |
| | 7.70 − 7.63 (m, 2H), 7.50 (d, J = 15.9 | |
| | Hz, 1H), 7.35 (dd, J = 22.7, 9.7 Hz, | |
| | 2H), 7.28 (dd, J = 8.0, 1.5 Hz, 1H), | |
| | 6.81 (d, J = 8.1 Hz, 1H), 6.57 (d, J = | |
| | 15.9 Hz, 1H), 6.47 (d, J = 8.5 Hz, 2H), | |
| | 4.22 (d, J = 12.9 Hz, 2H), 4.01 (s, 2H), | |
| | 3.71 (s, 3H), 3.38 (m, 1H),3.15 (t, J = | |
| | 12.3 Hz, 2H), 2.08 (s, 2H), 1.57 (s, | |
| | 2H). | |
| 201 | ¹H NMR (400 MHz, Methanol-d₄) δ | 639.3 |
| | 8.49 (s, 1H), 8.34 (s, 1H), 7.65 (dd, | |
| | J = 8.0, 6.6 Hz, 1H), 7.32 − 7.23 (m, | |
| | 2H), 7.21 − 7.10 (m, 3H), 6.78 − 6.68 | |
| | (m, 1H), 6.43 − 6.39 (m, 2H), 4.35 (d, | |
| | J = 13.5 Hz, 2H), 4.15 (s, 2H), 3.72 (s, | |
| | 3H), 3.40 − 3.28 (m, 2H), 3.13 − 3.01 | |
| | (m, 2H), 2.89 (t, J = 7.5 Hz, 1H), 2.31 | |
| | (t, J = 7.5 Hz, 2H), 2.20 (d, J = 12.5 | |
| | Hz, 2H), 1.80 − 1.68 (m, 2H). | |
| 202 | ¹H NMR (400 MHz, DMSO-d₆) δ | 610.3 |
| | 9.86 (s, 2H), 9.09 (s, 2H), 8.51 (s, | |
| | 1H), 8.13 (s, 1H), 7.90 (dd, J = 8.0, | |
| | 6.8 Hz, 1H), 7.44 (dd, J = 10.0, 1.6 | |
| | Hz, 1H), 7.38 (d, J = 15.6 Hz, 1H), | |
| | 7.21 (d, J = 15.6 Hz, 1H), 7.12 (dd, | |
| | J = 7.6, 1.6 Hz, 1H), 6.78 (d, J = 8.4 | |
| | Hz, 1H), 6.56 − 6.30 (m, 2H), 4.30 (t, | |
| | J = 6.0 Hz, 2H), 3.71 (s, 3H), 3.64 (d, | |
| | J = 12.4 Hz, 2H), 3.37 (s, 1H), 3.02 − | |
| | 2.85 (m, 2H), 2.28 (d, J = 11.6 Hz, | |
| | 2H), 2.01 (s, 3H), 1.92 (d, J = 11.6 | |
| | Hz, 2H). | |
| 203 | ¹H NMR (400 MHz, Methanol-d₄) | 674.3 |
| | δ ppm 8.45 (d, J = 1.6 Hz, 1H), 8.44 (s, | |
| | 1H), 7.74 (dd, J = 8.0, 6.6 Hz, 1H), | |
| | 7.68-7.49 (m, 5H), 7.36 (dd, J = 9.8, | |
| | 1.6 Hz, 1H), 7.24 (dd, J = 8.0, 1.6 Hz, | |
| | 1H), 6.90 (d, J = 8.0 Hz, 1H), 6.59- | |
| | 6.48 (m, 3H), 4.44 (d, J = 13.6 Hz, | |
| | 2H), 4.21 (s, 2H), 3.87-3.72 (m, 4H), | |
| | 3.34 (s, 1H), 3.17 (t, J = 12.0 Hz, 2H), | |
| | 2.99-2.85 (m, 4H), 2.36-2.23 (m, 2H), | |
| | 1.81 (qd, J = 12.0, 4.2 Hz, 2H). | |
| 204 | ¹H NMR (400 MHz, DMSO-d₆) δ | 680.3 |
| | ppm: 10.07 (s, 2H), 9.05 (d, J = 6.4 | |
| | Hz, 1H), 8.69 (d, J = 8.6 Hz, 1H), 8.51 | |
| | (s, 1H), 8.46 (s, 1H), 8.05 − 7.87 (m, | |
| | 2H), 7.68 (dd, J = 10.0, 1.5 Hz, 1H), | |
| | 7.31 (dd, J = 7.9, 1.5 Hz, 1H), 6.77 (d, | |
| | J = 8.4 Hz, 1H), 6.52 (d, J = 2.2 Hz, | |
| | 1H), 6.44 (dd, J = 8.2, 2.2 Hz, 1H), | |
| | 4.56 (s, 1H), 4.40 (d, J = 6.1 Hz, 2H), | |
| | 4.35 (d, J = 13.3 Hz, 2H), 3.65 (s, 2H), | |
| | 3.44 (s, 2H), 3.24 (t, J = 7.2 Hz, 2H), | |
| | 3.15 (t, J = 12.5 Hz, 2H), 2.58 (t, J = | |
| | 7.2 Hz, 1H), 2.36 − 2.23 (m, 2H), 1.94 − | |
| | 1.80 (m, 2H), 1.18 (s, 6H). | |
| 205 | ¹H NMR (400 MHz, DMSO-d₆) δ | 678.3 |
| | 10.83 (s, 1H), 9.09 (s, 1H), 8.77 (s, | |
| | 1H), 8.44 (s, 1H), 8.13 (s, 1H), 8.04 | |
| | (d, J = 8.2 Hz, 1H), 7.96 (dd, J = 8.0, | |
| | 6.8 Hz, 1H), 7.66 (dd, J = 10.1, 1.5 Hz, | |
| | 1H), 7.60 − 7.46 (m, 2H), 7.29 (dd, J = | |
| | 8.0, 1.4 Hz, 1H), 6.77 (d, J = 8.4 Hz, | |
| | 1H), 6.59 (d, J = 5.8 Hz, 1H), 6.50 (d, | |
| | J = 2.2 Hz, 1H), 6.43 (dd, J = 8.3, 2.2 | |
| | Hz, 1H), 4.70 (s, 1H), 4.33 − 4.14 (m, | |
| | 4H), 3.65 (s, 2H), 3.15 − 3.10 (m, 3H), | |
| | 2.13 (d, J = 12.2 Hz, 2H), 1.64 (d, J = | |
| | 12.0 Hz, 2H), 1.18 (s, 6H). | |
| 206 | ¹H NMR (400 MHz, DMSO-d₆) δ | 635.2 |
| | ppm 10.83 (s, 1H), 9.0 (s, 1H), 8.85 | |
| | (s, 2H), 8.51 (s, 1H), 8.40 (s, 1H), 7.73 | |
| | (dd, J = 8.0, 6.6 Hz, 1H), 7.56 (d, J = | |
| | 15.4 Hz, 1H), 7.35 (dd, J = 9.8, 1.6 | |
| | Hz, 1H), 7.27 −7.14 (m, 2H), 6.88- | |
| | 6.75 (m, 1H), 6.56-6.47 (m, 2H), 3.95 | |
| | (s, 4H), 3.81 (s, 3H). 3.73-3.60 (m, | |
| | 2H), 1.97-1.78 (m, 4H), 1.39 (s, 3H). | |
| 207 | ¹H NMR (400 MHz, DMSO-d₆) | 666.3 |
| | δ10.83 (s, 1H), 9.63 (s, 2H), 8.13 (s, | |
| | 1H), 7.91 (dd, J = 8.0, 6.8 Hz, 1H), | |
| | 7.68 (d, J = 8.0 Hz, 2H), 7.63 (d, J = | |
| | 8.0 Hz, 2H), 7.54 − 7.39 (m, 2H), 7.14 | |
| | (dd, J = 8.0, 1.6 Hz, 1H), 6.75 (d, J = | |
| | 8.4 Hz, 1H), 6.56 (d, J = 15.6 Hz, 1H), | |
| | 6.49 (d, J = 2.4 Hz, 1H), 6.41 (dd, J = | |
| | 8.0, 2.4 Hz, 1H), 4.70 (s, 1H), 4.23 (t, | |
| | J = 6.0 Hz, 2H), 3.64 (s, 4H), 3.28 (s, | |
| | 1H), 3.02 − 2.86 (m, 2H), 2.26 (t, J = | |
| | 7.2 Hz, 2H), 2.01 (s, 3H), 1.93 (d, J = | |
| | 10.0 Hz, 2H), 1.18 (s, 6H). | |
| 208 | ¹H NMR (400 MHz, DMSO-d₆) δ | 602.2 |
| | 10.72 (s, 1H), 9.01 (s, 1H), 8.65 (dd, J = | |
| | 2.2, 0.9 Hz, 1H), 8.44 (s, 1H), 8.18 − | |
| | 8.08 (m, 1H), 8.05 (dd, J = 8.0, 2.2 | |
| | Hz, 1H), 7.53 (d, J = 7.9 Hz, 2H), 7.44 | |
| | (t, J = 8.2 Hz, 3H), 6.80 (d, J = 8.9 Hz, | |
| | 1H), 6.54 − 6.29 (m, 3H), 4.20 (d, J = | |
| | 13.2 Hz, 2H), 3.86 (s, 2H), 3.71 (s, | |
| | 3H), 3.18 (t, J = 11.9 Hz,3H), 2.81 (s, | |
| | 1H), 2.02 (d, J = 12.4 Hz, 2H), 1.49 | |
| | (q, J = 11.3 Hz, 2H). | |
| 209 | ¹H NMR (400 MHz, DMSO-d₆) δ | 610.3 |
| | 10.45 (s, 1H), 9.61 (s, 2H), 8.51 (s, | |
| | 1H), 8.12 (s, 1H), 7.91 (t, J = 7.6 Hz, | |
| | 1H), 7.55 (d, J = 7.6 Hz, 2H), 7.46 | |
| | (dd, J = 10.0, 1.6 Hz, 1H), 7.27 (d, J = | |
| | 7.6 Hz, 2H), 7.12 (dd, J = 8.0, 1.6 | |
| | Hz, 1H), 6.79 (d, J = 8.4 Hz, 1H), | |
| | 6.51 − 6.40 (m, 2H), 4.16 (t, J = 5.6 | |
| | Hz, 2H), 3.71 (s, 3H), 3.66 (d, J = | |
| | 12.8 Hz, 2H), 3.35-3.32 (m, 1H), | |
| | 3.06 − 2.90 (m, 2H), 2.84 (t, J = 7.6 | |
| | Hz, 2H), 2.28 (q, J = 7.2, 6.8 Hz, | |
| | 4H), 2.01 (s, 3H), 1.98 − 1.87 (m, | |
| | 2H). | |
| 210 | ¹H NMR (400 MHz, Methanol-d₄) δ | 633.3 |
| | 8.57 (s, 1H), 8.48 (s, 1H), 7.91 (dd, J = | |
| | 8.0, 6.6 Hz, 1H), 7.82 (d, J = 7.9 Hz, | |
| | 1H), 7.77 (d, J = 15.8 Hz, 2H), 7.68 | |
| | (d, J = 8.0 Hz, 2H), 7.52 (dd, J = 9.8, | |
| | 1.5 Hz, 1H), 7.39 (dd, J = 8.0, 1.5 Hz, | |
| | 1H), 7.02 − 6.95 (m, 1H), 6.72 − 6.67 | |
| | (m, 2H), 6.66 (t, J = 2.1 Hz, 1H), 4.57 | |
| | (s, 1H), 4.51 (d, J = 12.5 Hz, 2H), 3.81 | |
| | (s, 3H), 3.24 (d, J = 12.8 Hz, 1H), 3.19 − | |
| | 3.09 (m, 2H), 2.31 (dd, J = 74.1, | |
| | 12.5 Hz, 2H), 1.93 − 1.85 (m, 2H), | |
| | 1.74 (d, J = 6.7 Hz, 3H). | |
| 211 | ¹H NMR (400 MHz, Methanol-d₄) δ | 633.3 |
| | 8.41 (s, 1H), 8.32 (s, 1H), 7.64 (dd, J = | |
| | 8.0, 6.6 Hz, 1H), 7.44 (d, J = 15.8 | |
| | Hz, 1H), 7.34 (d, J = 3.8 Hz, 3H), 7.26 | |
| | (dd, J = 9.8, 1.5 Hz, 1H), 7.13 (dd, J = | |
| | 8.0, 1.5 Hz, 1H), 6.76 − 6.67 (m, 1H), | |
| | 6.46 − 6.33 (m, 3H), 4.33 (d, J = 13.3 | |
| | Hz, 2H), 4.03 (s, 2H), 3.71 (s, 3H), | |
| | 3.08 (q, J = 12.3, 11.9 Hz, 2H), 2.55 | |
| | (s, 1H), 2.35 (s, 3H), 2.23 − 2.10 (m, | |
| | 2H), 1.76 − 1.59 (m, 2H). | |
| 212 | ¹H NMR (400 MHz, DMSO-d₆) δ | 691.3 |
| | 10.63 (s, 1H), 9.18 (s, 1H), 8.71 (s, | |
| | 1H), 8.46 (s, 1H), 7.97 (dd, J = 8.0, 6.8 | |
| | Hz, 1H), 7.68 (dd, J = 10.0, 1.4 Hz, | |
| | 1H), 7.63 − 7.51 (m, 4H), 7.30 (dd, J = | |
| | 8.0, 1.4 Hz, 1H), 6.78 (d, J = 8.4 Hz, | |
| | 1H), 6.51 (d, J = 2.2 Hz, 1H), 6.44 (dd, | |
| | J = 8.2, 2.2 Hz, 1H), 6.07 (d, J = 1.6 | |
| | Hz, 1H), 4.32 (d, J = 13.0 Hz, 2H), | |
| | 4.24 (s, 2H), 3.65 (s, 2H), 3.26 (s, 1H), | |
| | 3.13 (t, J = 12.6 Hz, 2H), 2.52 (s, 3H), | |
| | 2.27 (d, J = 11.4 Hz, 2H), 1.88 − 1.71 | |
| | (m, 2H), 1.18 (s, 6H). | |
| 213 | ¹H NMR (400 MHz, Methanol-d₄) δ | 634.2 |
| | 8.64 (s, 1H), 8.41 (s, 1H), 8.20 (s, 1H), | |
| | 7.97 − 7.88 (m, 1H), 7.73 (dd, J = 8.0, | |
| | 6.6 Hz, 1H), 7.60 (d, J = 6.6 Hz, 1H), | |
| | 7.57 (s, 1H), 7.36 (dd, J = 9.8, 1.5 Hz, | |
| | 1H), 7.23 (dd, J = 8.0, 1.5 Hz, 1H), | |
| | 6.88 (d, J = 5.4 Hz, 1H), 6.81 (d, J = | |
| | 8.8 Hz, 1H), 6.55 − 6.45 (m, 2H), 4.11 − | |
| | 3.95 (m, 4H), 3.81 (s, 3H), 3.66 − | |
| | 3.53 (m, 2H), 1.94 (t, J = 6.6 Hz, 2H), | |
| | 1.88 (s, 2H), 1.43 (s, 3H). | |
| 214 | ¹H NMR (400 MHz, DMSO-d₆) δ | 687.2 |
| | 10.80 (s, 1H), 10.25 (s, 1H), 9.07 (d, | |
| | J = 2.4 Hz, 1H), 8.96 (s, 2H), 8.53 (s, | |
| | 1H), 7.99 (dd, J = 8.0, 6.8 Hz, 1H), | |
| | 7.76-7.55 (m, 3H), 7.45 (dd, J = 8.0, | |
| | 1.6 Hz, 1H), 7.30 (dd, J = 8.0, 1.6 Hz, | |
| | 1H), 7.18 (dd, J = 8.4, 1.4 Hz, 1H), | |
| | 6.67 (d, J = 2.4 Hz, 1H), 6.64 − 6.43 | |
| | (m, 2H), 4.37 − 4.14 (m, 4H), 3.35 (s, | |
| | 2H), 2.04 (d, J = 14.4 Hz, 4H), 1.59 (s, | |
| | 3H). | |
| 215 | ¹H NMR (400 MHz, Methanol-d₄) δ | 691.3 |
| | ppm 8.55 (s, 1H), 8.44 (s, 1H), 7.75 | |
| | (dd, J = 8.0, 6.6 Hz, 1H), 7.66-7.49 | |
| | (m, 5H), 7.37 (dd, J =9.6, 1.6 Hz, 1H), | |
| | 7.24 (dd, J = 8.0, 1.6 Hz, 1H), 6.80 (d, | |
| | J = 8.2 Hz, 1H), 6.57-6.45 (m, 3H), | |
| | 4.23 (d, J = 13.8 Hz, 2H), 4.13 (s, 2H), | |
| | 3.78 (s, 2H), 3.46 (t, J = 12.4 Hz, 2H), | |
| | 2.11-1.95 (m, 4H), 1.56 (s, 3H), 1.30 | |
| | (s, 6H). | |
| 216 | ¹H NMR (400 MHz, Methanol-d₄) δ | 639.2 |
| | 8.51 (s, 1H), 8.33 (s, 1H), 7.64 (dd, J = | |
| | 8.0, 6.6 Hz, 1H), 7.39 (d, J = 8.0 Hz, | |
| | 2H), 7.33 (d, J = 8.0 Hz, 2H), 7.27 (dd, | |
| | J = 9.8, 1.5 Hz, 1H), 7.13 (dd, J = 8.0, | |
| | 1.5 Hz, 1H), 6.77 − 6.65 (m, 1H), 6.46 − | |
| | 6.35 (m, 2H), 4.32 (d, J = 13.3 Hz, | |
| | 2H), 4.03 (s, 2H), 3.71 (s, 3H), 3.46 (s, | |
| | 2H), 3.15 (d, J = 10.9 Hz, 1H), 3.12 − | |
| | 3.00 (m, 2H), 2.15 (d, J = 12.3 Hz, | |
| | 2H), 1.78 − 1.57 (m, 2H). | |
| 217 | ¹H NMR (400 MHz, DMSO-d₆) δ | 634.3 |
| | ppm: 10.88 (s, 1H), 8.95 (s, 1H), 8.61 | |
| | (d, J = 2.1 Hz, 1H), 8.31 (s, 1H), 8.15 | |
| | (s, 1H), 7.94 (dd, J = 8.0, 6.9 Hz, 1H), | |
| | 7.84 (dd, J = 8.0, 2.2 Hz, 1H), 7.59 − | |
| | 7.43 (m, 3H), 7.18 (dd, J = 8.0, 1.5 Hz, | |
| | 1H), 6.92 (d, J = 15.4 Hz, 1H), 6.83 − | |
| | 6.74 (m, 1H), 6.53 − 6.43 (m, 2H), | |
| | 3.90 (s, 2H), 3.76 (d, J = 1.6 Hz, 2H), | |
| | 3.71 (s, 3H), 3.36 (d, J = 12.5 Hz, 2H), | |
| | 2.89 (q, J = 11.4 Hz, 2H), 2.71 (d, J = | |
| | 11.4 Hz, 1H), 2.01 (t, J = 7.4 Hz, 2H), | |
| | 1.60 (s, 2H). | |
| 218 | ¹H NMR (400 MHz, DMSO-d₆) δ | 691.3 |
| | ppm: 10.75 (s, 1H), 9.04 (s, 1H), 8.64 | |
| | (s, 1H), 8.32 (s, 1H), 8.15 (s, 1H), 7.95 | |
| | (dd, J = 8.0, 6.9 Hz, 1H), 7.57 (d, J = | |
| | 7.9 Hz, 2H), 7.54 − 7.41 (m, 4H), 7.19 | |
| | (dd, J = 8.0, 1.4 Hz, 1H), 6.75 (d, J = | |
| | 8.4 Hz, 1H), 6.52 − 6.47 (m, 1H), 6.47 − | |
| | 6.41 (m, 2H), 4.72 (s, 1H), 3.99 (s, | |
| | 2H), 3.79 − 3.72 (m, 2H), 3.64 (s, 2H), | |
| | 3.40 (d, J = 12.5 Hz, 2H), 2.91 (q, J = | |
| | 11.2 Hz, 3H), 2.08 (d, J = 12.1 Hz, | |
| | 2H), 1.68 (s, 2H), 1.18 (s, 6H). | |
| 219 | ¹H NMR (400 MHz, DMSO-d₆) δ | 639.3 |
| | ppm: 10.93 (s, 1H), 9.05 (s, 1H), 9.01 | |
| | (s, 1H), 8.46 (s, 1H), 7.97 (t, J = 7.4 | |
| | Hz, 1H), 7.67 (dd, J = 10.1, 1.4 Hz, | |
| | 1H), 7.46 (d, J = 7.6 Hz, 2H), 7.36 − | |
| | 7.25 (m, 3H), 6.81 (d, J = 8.1 Hz, 1H), | |
| | 6.47 (d, J = 8.0 Hz, 2H), 5.07 (ddd, J = | |
| | 48.3, 8.0, 4.4 Hz, 1H), 4.29 (d, J = | |
| | 13.0 Hz, 2H), 4.15 (s, 2H), 3.72 (s, | |
| | 3H), 3.20 − 3.03 (m, 4H), 2.79 (s, 1H), | |
| | 2.21 (s, 2H), 1.71 (s, 2H). | |
| 220 | ¹H NMR (400 MHz, Methanol-d₄) δ | 634.3 |
| | ppm 8.71 (d, J = 2.2 Hz, 1H), 8.41 (s, | |
| | 1H) 7.94 (d, J = 8.2 Hz, 1H), 7.62 − | |
| | 7.49 (m, 2H), 7.38 − 7.29 (m, 2H), | |
| | 7.25 (dd, J = 8.0, 1.6 Hz, 1H), 7.16 (d, | |
| | J = 1.6 Hz, 1H), 6.93 (d, J = 8.8 Hz, | |
| | 1H), 6.63 (h, J = 2.2 Hz, 2H), 4.60 (d, | |
| | J = 13.6 Hz, 2H), 4.20 (s, 2H), 3.88 (s, | |
| | 3H), 3.28 (s, 1H), 3.07 (t, J = 12.7 Hz, | |
| | 2H), 2.27 (d, J = 1.5 Hz, 3H), 2.23 (d, | |
| | J = 12.1 Hz, 2H), 1.61 (q, J = 11.9 Hz, | |
| | 2H). | |
| 221 | ¹H NMR (400 MHz, DMSO-d₆) δ | 692.3 |
| | ppm: δ 10.91 (s, 1H), 9.11 (s, 1H), | |
| | 8.71 (s, 1H), 8.63 (s, 1H), 8.33 (s, 1H), | |
| | 7.95 (t, J = 7.4 Hz, 2H), 7.64 (s, 1H), | |
| | 7.50 (d, J = 10.3 Hz, 2H), 7.19 (dd, J = | |
| | 8.0, 1.4 Hz, 1H), 6.96 (d, J = 15.4 | |
| | Hz, 1H), 6.75 (d, J = 8.4 Hz, 1H), 6.51 | |
| | (d, J = 2.2 Hz, 1H), 6.44 (dd, J = 8.2, | |
| | 2.2 Hz, 1H), 4.69 (s, 1H), 4.22 (s, 2H), | |
| | 3.75 (s, 2H), 3.65 (s, 2H), 3.44 (d, J = | |
| | 12.4 Hz, 2H), 2.95 (d, J = 12.6 Hz, | |
| | 2H), 2.71 (d, J = 11.4 Hz, 1H), 2.18 (s, | |
| | 2H), 1.82 (s, 2H), 1.18 (s, 6H). | |
| 222 | ¹H NMR (400 MHz, DMSO-) δ 10.35 | 655.2 |
| | (s, 1H), 8.98 (s, 1H), 8.69 (s, 1H), 8.43 | |
| | (d, J = 3.4 Hz, 1H), 7.66 (d, J = 10.4 | |
| | Hz, 1H), 7.44 (d, J = 15.9 Hz, 2H), | |
| | 7.34 (s, 1H), 7.28 (dd, J = 8.0, 1.5 Hz, | |
| | 1H), 7.23 (d, J = 8.0 Hz, 1H), 6.81 (d, | |
| | J = 8.1 Hz, 1H), 6.57 − 6.47 (m, 1H), | |
| | 6.46 (d, J = 1.6 Hz, 2H), 4.25 (d, J = | |
| | 13.2 Hz, 2H), 4.06 (s, 2H), 3.71 (s, | |
| | 3H), 3.17 (t, J = 12.8 Hz, 2H), 2.83 (t, | |
| | J = 7.4 Hz, 2H), 2.54 (s, 1H), 2.28 (t, | |
| | J = 7.5 Hz, 2H), 2.13 (s, 2H), 1.61 (s, | |
| | 2H). | |
| 223 | ¹H NMR (400 MHz, DMSO-d₆) δ | 705.3 |
| | 10.81 (s, 1H), 9.32 (s, 2H), 8.72 (s, | |
| | 1H), 8.41 (s, 1H), 7.95 (t, J = 7.6 Hz, | |
| | 1H), 7.77 (d, J = 8.0 Hz, 2H), 7.72 − | |
| | 7.61 (m, 3H), 7.49 (d, J = 15.6 Hz, | |
| | 1H), 7.26 (dd, J = 8.0, 1.5 Hz, 1H), | |
| | 6.76 (d, J = 8.4 Hz, 1H), 6.62 − 6.46 | |
| | (m, 2H), 6.41 (dd, J = 8.0, 2.4 Hz, 1H), | |
| | 4.12 (d, J = 13.2 Hz, 2H), 3.64 (s, 2H), | |
| | 3.29 (s, 1H), 3.05 − 2.91 (m, 2H), 1.81 | |
| | (s, 6H), 1.72 (d, J = 7.6 Hz, 4H), 1.17 | |
| | (s, 6H). | |
| 224 | ¹H NMR (400 MHz, DMSO-d₆) δ | 687.3 |
| | 10.78 (s, 1H), 8.68 (d, J = 2.2 Hz, 1H), | |
| | 8.57 (s, 1H), 8.18 (s, 1H), 7.96 (dd, | |
| | J = 8.0, 2.2 Hz, 1H), 7.90 (t, J = 7.4 | |
| | Hz, 1H), 7.82 (s, 1H), 7.75 − 7.66 (m, | |
| | 2H), 7.59 − 7.41 (m, 2H), 7.34 (dd, J = | |
| | 8.0, 1.4 Hz, 1H), 7.14 (dd, J = 8.6, | |
| | 1.4 Hz, 1H), 6.54 (d, J = 15.9 Hz, 1H), | |
| | 4.75 (s, 1H), 4.55 (s, 2H), 4.24 (d, J = | |
| | 13.0 Hz, 2H), 3.93 (s, 2H), 3.22 (d, J = | |
| | 11.6 Hz, 2H), 2.79 (s, 1H), 2.08 − | |
| | 1.94 (m, 2H), 1.48 (q, J = 11.4 Hz, | |
| | 2H), 1.12 (s, 6H). | |
| 225 | ¹H NMR (400 MHz, DMSO-d₆) δ | 687.3 |
| | 10.88 (s, 1H), 8.63 (s, 1H), 8.58 (s, | |
| | 1H), 8.14 (s, 1H), 7.95 − 7.80 (m, 3H), | |
| | 7.72 (dd, J = 9.4, 6.8 Hz, 2H), 7.57 (d, | |
| | J = 7.8 Hz, 1H), 7.48 (d, J = 5.4 Hz, | |
| | 1H), 7.34 (d, J = 8.0 Hz, 1H), 7.13 (d, | |
| | J = 8.6 Hz, 1H), 6.92 (d, J = 5.4 Hz, | |
| | 1H), 4.76 (s, 1H), 4.55 (s, 2H), 4.27 | |
| | (d, J = 13.0 Hz, 2H), 3.95 (s, 2H), 3.22 | |
| | (t, J = 12.2 Hz, 2H), 2.90 (s, 1H), 2.07 | |
| | (d, J = 12.4 Hz, 2H), 1.54 (d, J = 11.6 | |
| | Hz, 2H), 1.12 (s, 6H). | |
| 226 | ¹H NMR (400 MHz, Methanol-d₄) δ | 722.3 |
| | ppm 8.51 (s, 1H), 8.49 (d, J = 3.4 Hz, | |
| | 1H), 7.78 (d, J = 5.2 Hz, 1H), 7.74 − | |
| | 7.66 (m, 1H), 7.67 − 7.41 (m, 6H), | |
| | 7.28 (d, J = 8.1 Hz, 1H), 6.51 (d, J = | |
| | 15.8 Hz, 1H), 4.71 (d, J = 21.2 Hz, | |
| | 2H), 4.57 (d, J = 13.7 Hz, 2H), 4.15 (s, | |
| | 2H), 3.23 (t, J = 12.4 Hz, 3H), 2.26 (d, | |
| | J = 12.4 Hz, 2H), 1.84 − 1.68 (m, 2H), | |
| | 1.25 (d, J = 4.9 Hz, 6H). | |
| 227 | ¹H NMR (400 MHz, DMSO-d₆) δ | 654.2 |
| | 10.92 (s, 1H), 9.14 (s, 1H), 8.98 (s, | |
| | 1H), 8.42 (s, 1H), 8.13 (s, 1H), 7.95 | |
| | (dd, J = 8.0, 6.8 Hz, 1H), 7.75 (s, 1H), | |
| | 7.66 (dd, J = 10.0, 1.4 Hz, 1H), 7.48 | |
| | (d, J = 5.4 Hz, 1H), 7.28 (dd, J = 8.0, | |
| | 1.6 Hz, 1H), 6.97 (d, J = 5.0 Hz, 1H), | |
| | 6.80 (d, J = 8.0 Hz, 1H), 6.50 − 6.43 | |
| | (m, 2H), 4.18 (d, J = 13.0 Hz, 2H), | |
| | 3.92 (s, 2H), 3.71 (s, 3H), 3.20 (t, J = | |
| | 12.0 Hz, 2H), 2.78 (s, 1H), 2.02 (d, J = | |
| | 12.4 Hz, 2H), 1.47 (d, J = 11.4 Hz, | |
| | 2H). | |
| 228 | ¹H NMR (400 MHz, DMSO-d₆) δ | 619.2 |
| | 10.83 (s, 1H), 9.60 (d, J = 5.6 Hz, 1H), | |
| | 9.47 (s, 1H), 8.35 (s, 1H), 8.25 (s, 1H), | |
| | 7.96 (t, J = 7.6 Hz, 1H), 7.72 − 7.55 | |
| | (m, 4H), 7.47 (d, J = 15.6 Hz, 1H), | |
| | 7.25 (d, J = 7.6 Hz, 1H), 6.79 (d, J = | |
| | 8.0 Hz, 1H), 6.55 (d, J = 15.6 Hz, 1H), | |
| | 6.49 − 6.35 (m, 2H), 4.19 (t, J = 5.6 | |
| | Hz, 2H), 3.95 (dd, J = 11.2, 6.8 Hz, | |
| | 1H), 3.85 (dd, J = 11.6, 7.6 Hz, 1H), | |
| | 3.76 (dt, J = 11.2, 7.6 Hz, 1H), 3.71 (s, | |
| | 3H), 3.55 (dd, J = 11.2, 7.2 Hz, 1H), | |
| | 3.07 (q, J = 6.4 Hz, 2H), 2.79 (d, J = | |
| | 7.2 Hz, 1H), 2.23 (dq, J = 12.0, 6.4 Hz, | |
| | 1H), 1.87 (dq, J = 12.2, 7.6 Hz, 1H). | |
| 229 | ¹H NMR (400 MHz, Methanol-d₄) δ | 689.3 |
| | 8.51 (s, 1H), 8.44 (s, 1H), 7.74 (t, J = | |
| | 7.3 Hz, 1H), 7.68 − 7.48 (m, 5H), 7.36 | |
| | (d, J = 9.6 Hz, 1H), 7.28 − 7.19 (m, | |
| | 1H), 6.78 (d, J = 8.3 Hz, 1H), 6.59 − | |
| | 6.43 (m, 3H), 4.27 (d, J = 13.7 Hz, | |
| | 2H), 4.17 (s, 2H), 3.96 (s, 2H), 3.43 (t, | |
| | J = 11.3 Hz, 2H), 2.14 − 1.95 (m, 4H), | |
| | 1.59 (s, 3H), 0.84 − 0.78 (m, 2H), 0.71 − | |
| | 0.65 (m, 2H). | |
| 230 | ¹H NMR (400 MHz, DMSO-d₆) δ | 638.2 |
| | 10.86 (s, 1H), 8.99 (s, 1H), 8.64 (s, | |
| | 1H), 8.42 (s, 1H), 8.14 (s, 1H), 8.02 − | |
| | 7.89 (m, 2H), 7.66 (d, J = 10.0 Hz, | |
| | 1H), 7.52 (d, J = 5.0 Hz, 1H), 7.28 (d, | |
| | J = 7.8 Hz, 1H), 6.81 (d, J = 8.0 Hz, | |
| | 1H), 6.63 (d, J = 10.0 Hz, 1H), 6.47 | |
| | (d, J = 8.2 Hz, 2H), 4.20 (d, J = 13.2 | |
| | Hz, 2H), 4.10 (s, 2H), 3.71 (s, 3H), | |
| | 3.17 (t, J = 12.0 Hz, 2H), 2.98 (s, 1H), | |
| | 2.06 (d, J = 12.2 Hz, 2H), 1.55 (d, J = | |
| | 11.0 Hz, 2H). | |

### Example 231

(E)-3-(4-(2-(4-(6-(4-Aminopiperidin-1-yl)-5-cyano-4-(4-cyano-3-fluorophenyl)pyridin-3-yl)-2-hydroxyphenoxy)ethyl)phenyl)-N-hydroxyacrylamide hydrochloride was prepared according to the synthetic method of Example 24, and the structure and characterization data are as follows:

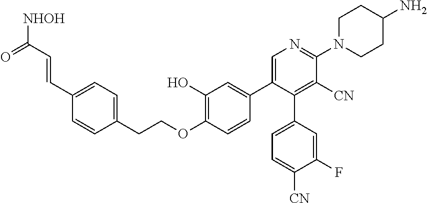

¹H NMR (400 MHZ, DMSO-d₆) δ 10.74 (s, 1H), 9.01 (s, 1H), 8.44 (s, 1H), 8.16 (s, 3H), 7.96 (dd, J=8.0, 6.8 Hz, 1H), 7.67 (dd, J=10.0, 1.4 Hz, 1H), 7.52-7.39 (m, 3H), 7.36 (d, J=8.0 Hz, 3H), 7.26 (dd, J=8.0, 1.4 Hz, 1H), 6.82 (d, J=8.2 Hz, 1H), 6.51-6.38 (m, 2H), 4.26 (d, J=13.4 Hz, 2H), 4.12 (t, J=6.8 Hz, 2H), 3.41-3.28 (m, 1H), 3.16 (t, J=12.2 Hz, 2H), 3.02 (t, J=6.8 Hz, 2H), 2.06 (d, J=11.2 Hz, 2H), 1.75-1.62 (m, 2H).

ESI-MS (m/z)=619.2 [M+H]⁺.

### Example 232

### Preparation of 2-(4-(1H-pyrazol-1-yl)phenyl)-6-((((1R,2S)-2-(4-fluorophenyl)cyclopropyl)amino)methyl)-N-(4-((E)-3-(hydroxyamino)-3-oxoprop-1-en-1-yl)benzyl)pyrimidine-4-carboxamide formate

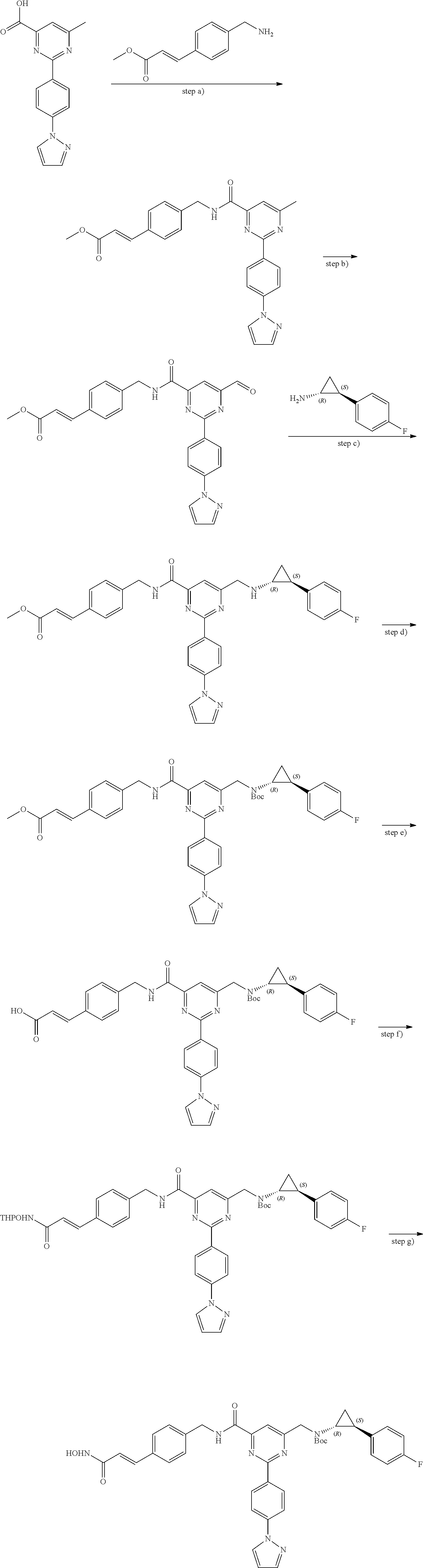

### Step a): preparation of methyl (E)-3-(4-((2-(4-(1H-pyrazol-1-yl)phenyl)-6-methylpyrimidine-4-carboxamido)methyl)phenyl)acrylate

2-(4-(1H-Pyrazol-1-yl)phenyl)-6-methylpyrimidine-4-carboxylic acid (500 mg, 1.8 mmol), methyl (E)-3-(4-(aminomethyl)phenyl)acrylate (344 mg, 1.8 mmol), HATU (821 mg, 2.2 mmol), and DIPEA (464 mg, 3.6 mmol) were added to a reaction flask containing DMF (5 mL), and the mixture was stirred at room temperature for 2 hours. The reactionsolution was quenched with water (10 mL), and then extracted with ethyl acetate (20 mL×2). The organic phases were combined, washed with saturated brine (15 mL×2), dried over anhydrous sodium sulfate, and concentrated under vacuum. The residue was purified by silica gel chromatography (eluent: DCM/MeOH=10/1) to afford methyl (E)-3-(4-((2-(4-(1H-pyrazol-1-yl)phenyl)-6-methylpyrimidine-4-carboxamido)methyl)phenyl)acrylate with a yield of 74% ESI-MS m/z=454.2 [M+H]⁺.

### Step b): preparation of methyl (E)-3-(4-((2-(4-(1H-pyrazol-1-yl)phenyl)-6-formylpyrimidine-4-carboxamido)methyl)phenyl)acrylate

Methyl (E)-3-(4-((2-(4-(1H-Pyrazol-1-yl)phenyl)-6-methylpyrimidine-4-carboxamido)methyl)phenyl)acrylate (605 mg, 1.3 mmol), selenium dioxide (432 mg, 3.9 mmol), and 1,4-dioxane (12 mL) were added to a reaction flask, and the mixture was stirred at 100° C. for 16 hours. After concentrating to dryness under reduced pressure, the residue was purified by silica gel chromatography (eluent: petroleum ether/ethyl acetate=1/2) to afford methyl (E)-3-(4-((2-(4-(1H-pyrazol-1-yl)phenyl)-6-formylpyrimidine-4-carboxamido)methyl)phenyl)acrylate with a yield of 52.5%

ESI-MS m/z=468.2 [M+H]⁺.

### Step c): preparation of methyl (E)-3-(4-((2-(4-(1H-pyrazol-1-yl)phenyl)-6-((((1R,2S)-2-(4-fluorophenyl)cyclopropyl)amino)methyl)pyrimidine-4-carboxamido)methyl)phenyl)acrylate

Methyl (E)-3-(4-((2-(4-(1H-pyrazol-1-yl)phenyl)-6-formylpyrimidine-4-carboxamido)methyl)phenyl)acrylate (319 mg, 683 μmol) and (1R,2S)-2-(4-fluorophenyl)cyclopropane-1-amine (127 mg, 683 μmol) were added to a reaction flask containing DCE (10 mL) and acetic acid (100 μL), and the mixture was stirred at room temperature for 2 hours. The reaction was monitored by LC-MS until the starting materials were consumed, then sodium cyanoborohydride (0.32 g, 2.3 mmol) was added at 0° C., and the mixture was stirred at room temperature for 2 hours. The reaction was quenched by adding ice cold and saturated solution of sodium bicarbonate, and then extracted with ethyl acetate (20 mL×2). The organic phases were combined, washed with saturated brine (15 mL×2), dried over anhydrous sodium sulfate, filtered and concentrated. The residue was purified by silica gel chromatography (eluent: petroleum ether/ethyl acetate=1/1) to afford methyl (E)-3-(4-((2-(4-(1H-pyrazol-1-yl)phenyl)-6-((((1R,2S)-2-(4-fluorophenyl)cyclopropyl)amino)methyl)pyrimidine-4-carboxamido)methyl)phenyl)acrylate with a yield of 58%.

ESI-MS m/z=603.2 [M+H]⁺.

### Step d): preparation of methyl (E)-3-(4-((2-(4-(1H-pyrazol-1-yl)phenyl)-6-(((tert-butoxycarbonyl) ((1R,2S)-2-(4-fluorophenyl)cyclopropyl)amino)methyl)pyrimidine-4-carboxamido)methyl)phenyl)acrylate

Methyl (E)-3-(4-((2-(4-(1H-pyrazol-1-yl)phenyl)-6-((((1R,2S)-2-(4-fluorophenyl)cyclopropyl)amino)methyl)pyrimidine-4-carboxamido)methyl)phenyl)acrylate (239 mg, 396 μmol) and triethylamine (1 mL) were dissolved in DCM (10 mL), then di-tert-butyl dicarbonate (131 mg, 594 μmol) was added, and the reaction was stirred at room temperature for 12 hours. After the reaction was completed, the reaction solution was concentrated to dryness, and the residue was purified by silica gel chromatography (eluent: petroleum ether/ethyl acetate=1/1) to afford methyl (E)-3-(4-((2-(4-(1H-pyrazol-1-yl)phenyl)-6-(((tert-butoxycarbonyl) ((1R,2S)-2-(4-fluorophenyl)cyclopropyl)amino)methyl)pyrimidine-4-carboxamido)methyl)phenyl)acrylate with a yield of 90%.

ESI-MS m/z=703.2 [M+H]⁺.

### Step e): preparation of (E)-3-(4-((2-(4-(1H-pyrazol-1-yl)phenyl)-6-(((tert-butoxycarbonyl) ((1R,2S)-2-(4-fluorophenyl)cyclopropyl)amino)methyl)pyrimidine-4-carboxamido)methyl)phenyl)acrylic acid

Methyl (E)-3-(4-((2-(4-(1H-pyrazol-1-yl)phenyl)-6-(((tert-butoxycarbonyl) ((1R,2S)-2-(4-fluorophenyl)cyclopropyl)amino)methyl)pyrimidine-4-carboxamido)methyl)phenyl)acrylate (251 mg, 356 μmol) and lithium hydroxide monohydrate (75 mg, 1.8 mmol) were added to a reaction flask containing THF (4 mL) and water (1 mL), and the mixture was stirred at room temperature for 2 hours. The pH of the solution was adjusted to 4 by dropwise addition of 2M hydrochloric acid aqueous solution, followed by extraction with MeOH/DCM (1:5) (20 mL×2). The organic solution was combined, washed with saturated brine (15 mL×2), dried with anhydrous sodium sulfate, and concentrated to afford (E)-3-(4-((2-(4-(1H-pyrazol-1-yl)phenyl)-6-(((tert-butoxycarbonyl) ((1R,2S)-2-(4-fluorophenyl)cyclopropyl)amino)methyl)pyrimidine-4-carboxamido)methyl)phenyl)acrylic acid, which was used directly for the next step.

ESI-MS m/z=689.2 [M+H]⁺.

### Step f): preparation of tert-butyl ((2-(4-(1H-pyrazol-1-yl)phenyl)-6-((4-((E)-3-oxo-3-((tetrahydro-2H-pyran-2-yl)oxy)amino)prop-1-en-1-yl)benzyl)carbamoyl)pyrimidin-4-yl)methyl)((1R,2S)-2-(4-fluorophenyl)cyclopropyl)carbamate

(E)-3-(4-((2-(4-(1H-pyrazol-1-yl)phenyl)-6-(((tert-butoxycarbonyl) ((1R,2S)-2-(4-fluorophenyl)cyclopropyl)amino)methyl)pyrimidine-4-carboxamido)methyl)phenyl)acrylic acid (245 mg, 356 μmol), O-(tetrahydro-2H-pyran-2-yl) hydroxylamine (62 mg, 534 μmol), HATU (162 mg, 427 μmol), and DIPEA (67 mg, 534 μmol) were added to a reaction flask containing DMF (5 mL), and the mixture was stirred at room temperature for 2 hours. The reaction was quenched with water (10 mL), followed by extraction with ethyl acetate (20 mL×2), and the organic phases were combined, washed with saturated brine (15 mL×2), dried with anhydrous sodium sulfate, and concentrated under vacuum. The residue was purified by silica gel chromatography (eluent: DCM/MeOH=10/1) to afford tert-butyl ((2-(4-(1H-pyrazol-1-yl)phenyl)-6-((4-((E)-3-oxo-3-((tetrahydro-2H-pyran-2-yl)oxy)amino)prop-1-en-1-yl)benzyl)carbamoyl)pyrimidin-4-yl)methyl)((1R,2S)-2-(4-fluorophenyl)cyclopropyl)carbamate with a yield of 48%.

ESI-MS m/z=788.3 [M+H]⁺.

### Step g): preparation of 2-(4-(1H-pyrazol-1-yl)phenyl)-6-((((1R,2S)-2-(4-fluorophenyl)cyclopropyl)amino)methyl)-N-(4-((E)-3-(hydroxyamino)-3-oxoprop-1-en-1-yl)benzyl)pyrimidine-4-carboxamide

Tert-butyl ((2-(4-(1H-pyrazol-1-yl)phenyl)-6-((4-((E)-3-oxo-3-((tetrahydro-2H-pyran-2-yl)oxy)amino)prop-1-en-1-yl)benzyl)carbamoyl)pyrimidin-4-yl)methyl)((1R,2S)-2-(4-fluorophenyl)cyclopropyl)carbamate (135 mg, 171 μmol) was added to a reaction flask, followed by hydrogen chloride solution in ethyl acetate (4M, 2.5 mL), and the mixture was stirred at room temperature for 1 hour, resulting in the precipitation of a significant amount of solid. After concentrated under reduced pressure, the obtained crude product was purified by Prep-HPLC (separation method 3) to afford 2-(4-(1H-pyrazol-1-yl)phenyl)-6-((((1R,2S)-2-(4-fluorophenyl)cyclopropyl)amino)methyl)-N-(4-((E)-3-(hydroxyamino)-3-oxoprop-1-en-1-yl)benzyl)pyrimidine-4-carboxamide formate with a yield of 22.5%.

¹H NMR (400 MHz, DMSO-d₆) δ 10.73 (s, 1H), 9.83 (t, J=6.4 Hz, 1H), 9.02 (s, 1H), 8.79-8.69 (m, 2H), 8.66 (d, J=2.6 Hz, 1H), 8.10-7.95 (m, 3H), 7.82 (d, J=1.6 Hz, 1H), 7.54 (d, J=7.8 Hz, 2H), 7.49-7.35 (m, 3H), 7.19-7.02 (m, 4H), 6.62 (t, J=2.0 Hz, 1H), 6.43 (d, J=15.8 Hz, 1H), 4.61 (d, J=6.4 Hz, 2H), 4.08 (s, 2H), 2.32 (dd, J=7.0, 3.6 Hz, 1H), 1.92 (ddd, J=9.2, 5.8, 2.4 Hz, 1H), 1.07 (dt, J=9.2, 4.6 Hz, 1H), 0.97 (q, J=5.8 Hz, 1H).

ESI-MS (m/z)=604.2 [M+H]⁺.

### Example 233

### Preparation of (E)-3-(4-(((1-(4-(4-cyano-3-fluorophenyl)-3-(cyanomethyl)-5-(3-hydroxy-4-methoxyphenyl)pyridin-2-yl)piperidin-4-yl)amino)methyl)phenyl)-N-hydroxyacrylamide formate

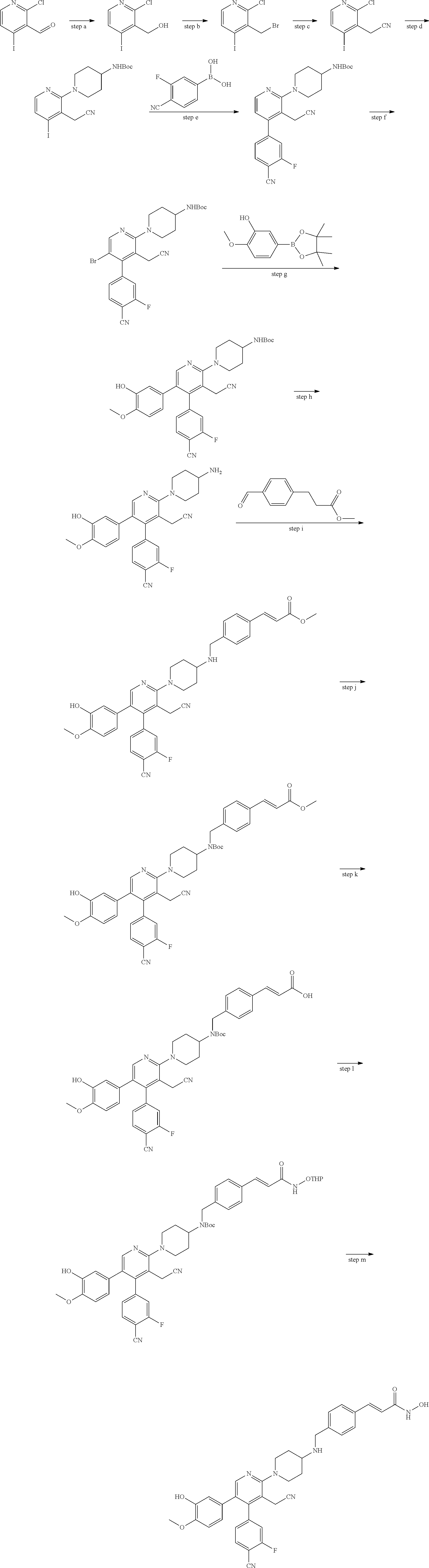

### Step a): preparation of (2-chloro-4-iodopyridin-3-yl)methanol

2-Chloro-4-iodopyridine-3-formaldehyde (2 g, 7.48 mmol) was added to a reaction flask containing methanol (15 mL). After cooling to 0° C., sodium borohydride (0.57 g, 15.03 mmol) was added, and the mixture was stirred at room temperature for 1 hour. The reaction solution was quenched by adding saturated aqueous solution of sodium bicarbonate (20 mL), followed by extraction with ethyl acetate (20 mL×2). And the organic phases were combined, washed with saturated brine (15 mL×2), dried over anhydrous sodium sulfate, filtered, and concentrated to afford (2-chloro-4-iodopyridin-3-yl)methanol with a yield of 89%

ESI-MS m/z=269.9 [M+H]⁺.

### Step b): preparation of 3-(bromomethyl)-2-chloro-4-iodopyridine

(2-Chloro-4-iodopyridin-3-yl)methanol (400 mg, 1.48 mmol), triphenylphosphine (1.16 g, 4.43 mmol), and carbon tetrabromide (1.47 g, 4.43 mmol) were added to a reaction flask containing DCM (20 mL), and the mixture was stirred at room temperature for 3 hours. The reactionsolution was concentrated after adding silica gel. The residue was purified with silica gel chromatography (eluent: petroleum ether/ethyl acetate=2/1) to afford 3-(bromomethyl)-2-chloro-4-iodopyridine with a yield of 81% ESI-MS m/z=331.8 [M+H]⁺.

### Step c): preparation of 2-(2-chloro-4-iodopyridin-3-yl)acetonitrile

3-(Bromomethyl)-2-chloro-4-iodopyridine (1.13 g, 3.40 mmol) and lithium hydroxide (0.17 g, 4.08 mmol) were added to a reaction flask containing acetonitrile (20 mL), and the reaction solution was cooled to 0° C. Then trimethylsilyl cyanide (0.40 g, 4.08 mmol) was added, and the mixture was stirred at room temperature for 4 hours. The reaction solution was quenched with water (20 mL), followed by extraction with ethyl acetate (20 mL×2). The organic phases were combined, washed with saturated brine (15 mL×2), dried over anhydrous sodium sulfate, and filtered. The residue was purified with silica gel chromatography (eluent: petroleum ether/ethyl acetate=1/1) to afford 2-(2-chloro-4-iodopyridin-3-yl)acetonitrile with a yield of 97%

ESI-MS m/z=278.9 [M+H]⁺.

### Step d): preparation of tert-butyl N-(1-(3-(cyanomethyl)-4-iodopyridin-2-yl)piperidin-4-yl)carbamate

2-(2-Chloro-4-iodopyridin-3-yl)acetonitrile (950 mg, 3.41 mmol), 4-Boc-aminopiperidine (1.37 g, 6.82 mmol), and DIEA (1.32 g, 10.23 mmol) were added to a reaction flask containing NMP (8 mL), and the mixture was stirred at 70° C. for 4 hours. The reactionsolution was quenched with water (20 mL), followed by extraction with ethyl acetate (20 mL×2). The organic phases were combined, washed with saturated brine (15 mL×2), dried over anhydrous sodium sulfate, filtered and concentrated. The residue was purified by silica gel chromatography (eluent: petroleum ether/ethyl acetate=1/1) to afford tert-butyl N-(1-(3-(cyanomethyl)-4-iodopyridin-2-yl)piperidin-4-yl)carbamate with a yield of 19%.

ESI-MS m/z=442.2 [M+H]⁺.

### Step e): preparation of tert-butyl N-(1-(4-(4-cyano-3-fluorophenyl)-3-(cyanomethyl)pyridin-2-yl)piperidin-4-yl)carbamate

Tert-butyl N-(1-(3-(cyanomethyl)-4-iodopyridin-2-yl)piperidin-4-yl)carbamate (270 mg, 0.61 mmol), 4-cyano-3-fluorophenylboronic acid (0.12 g, 0.73 mmol), Pd(dppf)Cl₂ (0.045 g, 0.061 mmol), and cesium carbonate (0.40 g, 1.22 mmol) were added to a microwave tube containing 1,4-dioxane (4 mL) and water (1 mL), and the mixture was stirred at 80° C. for 30 minutes. The reaction solution was concentrated and the residue was purified by silica gel chromatography (eluent: petroleum ether/ethyl acetate=1/1) to afford tert-butyl N-(1-(4-(4-cyano-3-fluorophenyl)-3-(cyanomethyl)pyridin-2-yl)piperidin-4-yl)carbamate with a yield of 90%.

ESI-MS m/z=436.2 [M+H]⁺.

### Step f): preparation of tert-butyl N-(1-(5-bromo-4-(4-cyano-3-fluorophenyl)-3-(cyanomethyl)pyridin-2-yl)piperidin-4-yl)carbamate

Tert-butyl N-(1-(4-(4-cyano-3-fluorophenyl)-3-(cyanomethyl)pyridin-2-yl)piperidin-4-yl)carbamate (210 mg, 0.48 mmol) and 1-bromopyrrolidine-2,5-dione (0.13 g, 0.73 mmol) were added to a reaction flask containing acetonitrile (5 mL), and the mixture was stirred at 0° C. for 1 hour. The reaction solution was quenched with water (10 mL), followed by extraction with ethyl acetate (20 mL×2). The organic phases were combined, washed with saturated brine (15 mL×2), dried over anhydrous sodium sulfate, filtered and concentrated. The residue was purified by silica gel chromatography (eluent: petroleum ether/ethyl acetate=1/1) to afford tert-butyl N-(1-(5-bromo-4-(4-cyano-3-fluorophenyl)-3-(cyanomethyl)pyridin-2-yl)piperidin-4-yl)carbamate with a yield of 97% ESI-MS m/z=514.1 [M+H]⁺.

### Step g): preparation of tert-butyl N-(1-(4-(4-cyano-3-fluorophenyl)-3-(cyanomethyl)-5-(3-hydroxy-4-methoxyphenyl)pyridin-2-yl)piperidin-4-yl)carbamate

Tert-butyl N-(1-(5-bromo-4-(4-cyano-3-fluorophenyl)-3-(cyanomethyl)pyridin-2-yl)piperidin-4-yl)carbamate (230 mg, 0.45 mmol), 2-methoxy-5-(tetramethyl-1,3,2-dioxaborolan-2-yl) phenol (0.14 g, 0.54 mmol), Pd(dppf)Cl₂ (0.033 g, 0.045 mmol), and cesium carbonate (0.29 g, 0.90 mmol) were added to a microwave tube containing 1,4-dioxane (2 mL) and water (0.4 mL), and the mixture was stirred at 100° C. for 30 minutes. The reaction liquid was concentrated and the residue was purified by silica gel chromatography (eluent: petroleum ether/ethyl acetate=1/1) to afford tert-butyl N-(1-(4-(4-cyano-3-fluorophenyl)-3-(cyanomethyl)-5-(3-hydroxy-4-methoxyphenyl)pyridin-2-yl)piperidin-4-yl)carbamate with a yield of 64%.

ESI-MS m/z=558.2 [M+H]⁺.

### Step h): preparation of 4-(2-(4-aminopiperidin-1-yl)-3-(cyanomethyl)-5-(3-hydroxy-4-methoxyphenyl)pyridin-4-yl)-2-fluorobenzonitrile

Tert-butyl N-(1-(4-(4-cyano-3-fluorophenyl)-3-(cyanomethyl)-5-(3-hydroxy-4-methoxyphenyl)pyridin-2-yl)piperidin-4-yl)carbamate (160 mg, 0.29 mmol) and hydrochloric acid solution in ethyl acetate (4M, 10 mL) were added to a reaction flask, and the mixture was stirred at room temperature for 1 hour. The pH of the reaction solution was adjusted to 8 with saturated aqueous solution of NaHCO₃, followed by extraction with ethyl acetate (20 mL×2). The organic phases were combined, washed with saturated brine (15 mL×2), dried over anhydrous sodium sulfate, and filtered, concentrated to afford 4-(2-(4-aminopiperidin-1-yl)-3-(cyanomethyl)-5-(3-hydroxy-4-methoxyphenyl)pyridin-4-yl)-2-fluorobenzonitrile with a yield of 98%.

ESI-MS m/z=458.2 [M+H]⁺.

### Step i): preparation of methyl (E)-3-(4-{[(1-(4-(4-cyano-3-fluorophenyl)-3-(cyanomethyl)-5-(3-hydroxy-4-methoxyphenyl)pyridin-2-yl)piperidin-4-yl)amino]methyl}phenyl)prop-2-enoate

4-(2-(4-Aminopiperidin-1-yl)-3-(cyanomethyl)-5-(3-hydroxy-4-methoxyphenyl)pyridin-4-yl)-2-fluorobenzonitrile (135 mg, 0.30 mmol) and methyl (E)-3-(4-formylphenyl)acrylate (0.11 g, 0.60 mmol) were added to a reaction flask containing DCE (5 mL). The mixture was stirred at room temperature for 1 hour, then the reaction solution was cooled to 0° C. and sodium cyanoborohydride (0.075 g, 1.2 mmol) was added, and the mixture was stirred at room temperature for 18 hours. The reaction solution was quenched with water (20 mL), extracted with ethyl acetate (20 mL×2). The organic phases were combined, washed with saturated brine (15 mL×2), dried over anhydrous sodium sulfate, filtered and concentrated. The residue was purified by silica gel chromatography (eluent: DCM/MeOH=10/1) to afford methyl (E)-3-(4-{[(1-(4-(4-cyano-3-fluorophenyl)-3-(cyanomethyl)-5-(3-hydroxy-4-methoxyphenyl)pyridin-2-yl)piperidin-4-yl)amino methyl}phenyl)prop-2-enoate with a yield of 40%. ESI-MS m/z=632.3 [M+H]⁺.

### Step j): preparation of methyl (E)-3-(4-({[(tert-butoxy)carbonyl](1-(4-(4-cyano-3-fluorophenyl)-3-(cyanomethyl)-5-(3-hydroxy-4-methoxyphenyl)pyridin-2-yl)piperidin-4-yl)amino}methyl)phenyl)prop-2-enoate

Methyl (E)-3-(4-{[(1-(4-(4-cyano-3-fluorophenyl)-3-(cyanomethyl)-5-(3-hydroxy-4-methoxyphenyl)pyridin-2-yl)piperidin-4-yl)amino]methyl}phenyl)prop-2-enoate (75 mg, 0.12 mmol), di-tert-butyl dicarbonate (0.13 g, 0.60 mmol), and triethylamine (0.036 g, 0.36 mmol) were added to a reaction flask containing DCM (5 mL). The mixture was stirred at room temperature for 1 hour, and the reaction solution was concentrated to afford methyl (E)-3-(4-({[tert-butoxycarbonyl](1-(4-(4-cyano-3-fluorophenyl)-3-(cyanomethyl)-5-(3-hydroxy-4-methoxyphenyl)pyridin-2-yl)piperidin-4-yl)amino}methyl)phenyl)prop-2-enoate with a yield of 97%.

ESI-MS m/z=732.3 [M+H]⁺.

### Step k): preparation of (E)-3-(4-(((tert-butoxycarbonyl)(1-(4-(4-cyano-3-fluorophenyl)-3-(cyanomethyl)-5-(3-hydroxy-4-methoxyphenyl)pyridin-2-yl)piperidin-4-yl)amino)methyl)phenyl)acrylic acid

Methyl (E)-3-(4-({[(tert-butoxy)carbonyl](1-(4-(4-cyano-3-fluorophenyl)-3-(cyanomethyl)-5-(3-hydroxy-4-methoxyphenyl)pyridin-2-yl)piperidin-4-yl)amino)methyl)phenyl)prop-2-enoate (85 mg, 0.12 mmol) and lithium hydroxide (0.057 g, 2.38 mmol) were added to a reaction flask containing THF (5 mL), methanol (3 mL), and water (2 mL), and the mixture was stirred at room temperature for 1 hour. The pH of the reactionsolution was adjusted to 4 by dropwise addition of 2M HCl, followed by extraction with a mixture of DCM:MeOH=5:1 (20 mL×2). The organic phases were combined, washed with saturated brine (15 mL×2), dried over anhydrous sodium sulfate, filtered and concentrated to afford ((E)-3-(4-(((tert-butoxycarbonyl)(1-(4-(4-cyano-3-fluorophenyl)-3-(cyanomethyl)-5-(3-hydroxy-4-methoxyphenyl)pyridin-2-yl)piperidin-4-yl)amino)methyl)phenyl)acrylic acid with a yield of 81%.

ESI-MS m/z=718.3 [M+H]⁺.

### Step 1): preparation of tert-butyl (E)-(1-(4-(4-cyano-3-fluorophenyl)-3-(cyanomethyl)-5-(3-hydroxy-4-methoxyphenyl)pyridin-2-yl)piperidin-4-yl)(4-(3-oxo-3-(((tetrahydro-2H-pyran-2-yl)oxy)amino)prop-1-en-1-yl)benzyl)carbamate

(E)-3-(4-(((tert-butoxycarbonyl)(1-(4-(4-cyano-3-fluorophenyl)-3-(cyanomethyl)-5-(3-hydroxy-4-methoxyphenyl)pyridin-2-yl)piperidin-4-yl)amino)methyl)phenyl)acrylic acid (70 mg, 0.098 mmol), O-(tetrahydro-2H-pyran-2-yl) hydroxylamine (0.023 g, 0.20 mmol), HATU (0.056 g, 0.15 mmol), and DIEA (0.051 g, 0.39 mmol) were added to a reaction flask containing DMF (4 mL), and the mixture was stirred at room temperature for 1 hour. The reaction solution was quenched with water (10 mL), followed by extraction with ethyl acetate (10 mL×2). The organic phases were combined, washed with saturated brine (15 mL×2), dried over anhydrous sodium sulfate, and filtered. The residue was purified by silica gel chromatography (eluent: petroleum ether/ethyl acetate=1/1) to tert-butyl (E)-(1-(4-(4-cyano-3-fluorophenyl)-3-(cyanomethyl)-5-(3-hydroxy-4-methoxyphenyl)pyridin-2-yl)piperidin-4-yl)(4-(3-oxo-3-(((tetrahydro-2H-pyran-2-yl)oxy)amino)prop-1-en-1-yl)benzyl)carbamate with a yield of 62%.

ESI-MS m/z=817.4 [M+H]⁺.

### Step m): preparation of (E)-3-(4-(((1-(4-(4-cyano-3-fluorophenyl)-3-(cyanomethyl)-5-(3-hydroxy-4-methoxyphenyl)pyridin-2-yl)piperidin-4-yl)amino)methyl)phenyl)-N-hydroxyacrylamide formate

tert-butyl (E)-(1-(4-(4-cyano-3-fluorophenyl)-3-(cyanomethyl)-5-(3-hydroxy-4-methoxyphenyl)pyridin-2-yl)piperidin-4-yl)(4-(3-oxo-3-(((tetrahydro-2H-pyran-2-yl)oxy)amino)prop-1-en-1-yl)benzyl)carbamate (50 mg, 0.061 mmol) was added to a reaction flask containing a 4M hydrochloric acid solution in ethyl acetate (5 mL), and the mixture was stirred at room temperature for 30 minutes. The reaction solution was concentrated, and the residue was purified by Prep-HPLC (separation method 3) to afford (E)-3-(4-(((1-(4-(4-cyano-3-fluorophenyl)-3-(cyanomethyl)-5-(3-hydroxy-4-methoxyphenyl)pyridin-2-yl)piperidin-4-yl)amino)methyl)phenyl)-N-hydroxyacrylamide formate with a yield of 50%.

¹H NMR (400 MHZ, DMSO-d₆) δ ppm: 10.78 (s, 1H), 8.97 (s, 1H), 8.51 (s, 1H), 8.32 (d, J=2.1 Hz, 1H), 8.15 (d, J=0.7 Hz, 1H), 7.95 (dd, J=8.0, 6.9 Hz, 1H), 7.59 (d, J=8.0 Hz, 2H), 7.56-7.47 (m, 3H), 7.44 (s, 1H), 7.22-7.14 (m, 1H), 6.81-6.74 (m, 1H), 6.53-6.43 (m, 3H), 4.04 (s, 2H), 3.86 (s, 1H), 3.76 (d, J=1.8 Hz, 2H), 3.71 (s, 3H), 3.42 (t, J=13.1 Hz, 2H), 2.94 (dd, J=22.3, 11.0 Hz, 2H), 2.12 (d, J=12.1 Hz, 2H), 1.74 (s, 2H).

ESI-MS m/z=633.3 [M+H]⁺.

### Example 234

(E)-3-(5-(((1-(4-Cyano-6-(4-cyano-3-fluorophenyl)-5-(3-hydroxy-4-methoxyphenyl)pyridin-2-yl)piperidin-4-yl)amino)methyl)pyrimidin-2-yl)-N-hydroxyacrylamide formate was prepared according to the synthetic method of Example 176, and the structure and characterization data are as follows:

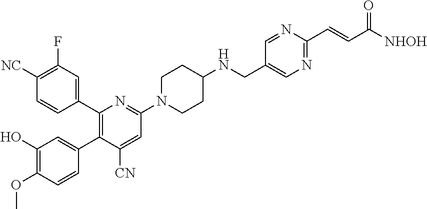

¹H NMR (400 MHz, Methanol-d₄) δ ppm 8.95 (s, 2H), 8.76 (s, 1H), 7.63-7.52 (m, 2H), 7.42-7.21 (m, 4H), 6.93 (d, J=8.8 Hz, 1H), 6.63 (dq, J=4.4, 2.2 Hz, 2H), 4.69 (d, J=13.8 Hz, 2H), 4.41 (s, 2H), 3.88 (s, 3H), 3.62 (t, J=11.4 Hz, 1H), 3.08 (d, J=12.8 Hz, 2H), 2.32 (d, J=11.6 Hz, 2H), 1.82-1.64 (m, 2H).

ESI-MS (m/z)=621.2 [M+H]⁺.

Examples 235-262 were prepared similarly according to the synthetic method of Example 37 (the separation method for the compounds: hydrochloride and formate were prepared by separation method 1 and 3, respectively), and the structure and characterization data are as follows:

| Example | Chemical name | Structure |
|---|---|---|
| 235 | (E)-3-(4-(1-((1- (3-Cyano-4-(4- cyano-3- fluorophenyl)-5- (3-hydroxy-4-(2- hydroxy-2- methylpropoxy) phenyl)pyridin-2- yl)piperidin-4- yl)amino)ethyl) phenyl)-N- hydroxyacrylamide formate | 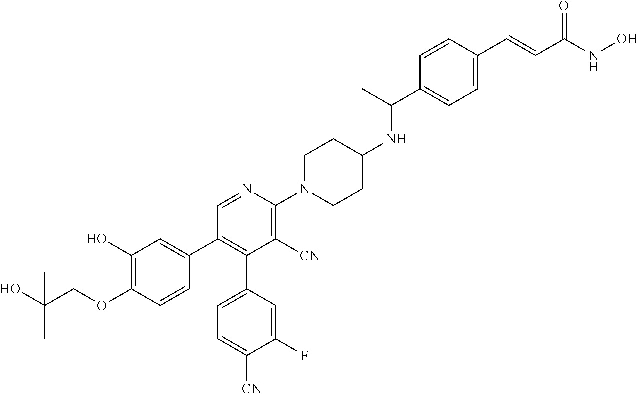 [SMILES: CC(NC1CCN(c2ncc(-c3ccc(OCC(C)(C)O)c(O)c3)c(-c3ccc(C#N)c(F)c3)c2C#N)CC1)c1ccc(/C=C/C(=O)NO)cc1] |
| 236 | (E)-3-(6-(((1-(3- Cyano-4-(4- cyano-3- fluorophenyl)-5- (5-fluoro-3- methylbenzo[d] isoxazol-6- yl)pyridin-2- yl)piperidin-4- yl)amino)methyl) pyridin-3-yl)-N- hydroxyacrylamide formate | 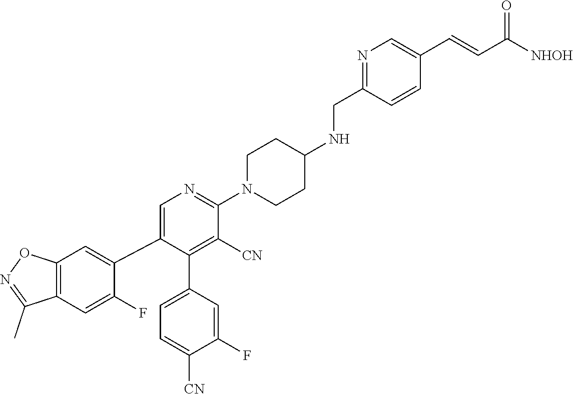 |
| 237 | (E)-3-(4-((1-(5′- (4-Chloro-3- hydroxyphenyl)- 3′,6-dicyano- [3,4′-bipyridin]- 2′-yl)piperidin-4- yl)amino)methyl) phenyl)-N- hydroxyacrylamide formate | 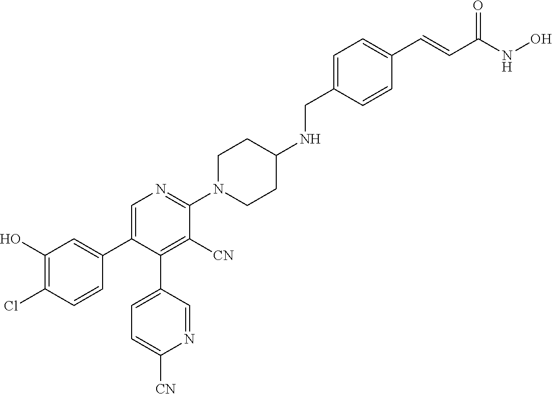 [SMILES: N#Cc1ccc(-c2c(-c3ccc(Cl)c(O)c3)cnc(N3CCC(NCc4ccc(/C=C/C(=O)NO)cc4)CC3)c2C#N)cn1] |
| 238 | (E)-3-(6-((1-(5′- (4-Chloro-3- hydroxyphenyl)- 3′,6-dicyano- [3,4′-bipyridin]- 2′-yl)piperidin-4- yl)amino)methyl) pyridin-3-yl)-N- hydroxyacrylamide formate | 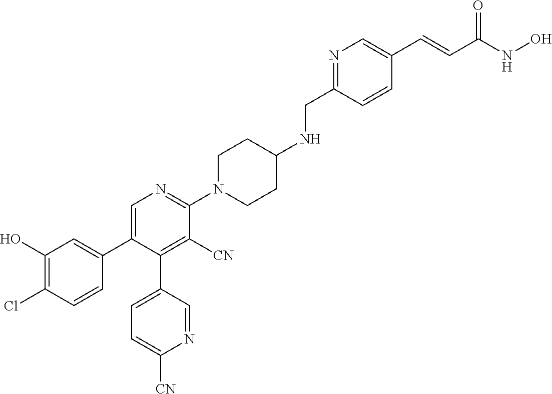 [SMILES: N#Cc1ccc(-c2c(-c3ccc(Cl)c(O)c3)cnc(N3CCC(NCc4ccc(/C=C/C(=O)NO)cn4)CC3)c2C#N)cn1] |
| 239 | (E)-3-(6-(((1-(5- (4-Chloro-3- hydroxyphenyl)- 3-cyano-4-(4- cyano-3- fluorophenyl) pyridin-2- yl)piperidin-4- yl)amino)methyl) pyridin-3-yl)-N- hydroxyacrylamide formate | 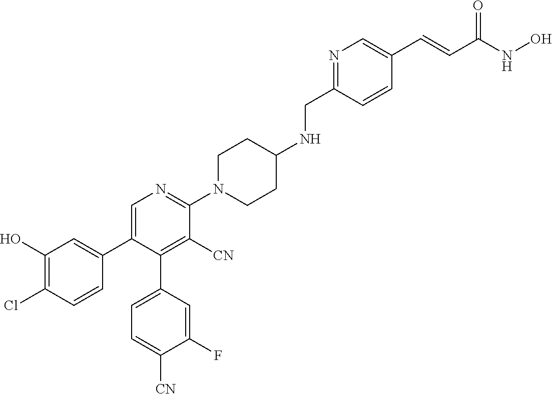 [SMILES: N#Cc1ccc(-c2c(-c3ccc(Cl)c(O)c3)cnc(N3CCC(NCc4ccc(/C=C/C(=O)NO)cn4)CC3)c2C#N)cc1F] |
| 240 | (E)-3-(6-(((1- (3′,6-Dicyano-5′- (2-fluoro-5- hydroxy-4- methoxyphenyl)- [3,4′-bipyridin]- 2′-yl)piperidin-4- yl)amino)methyl) pyridin-3-yl)-N- hydroxyacrylamide formate | 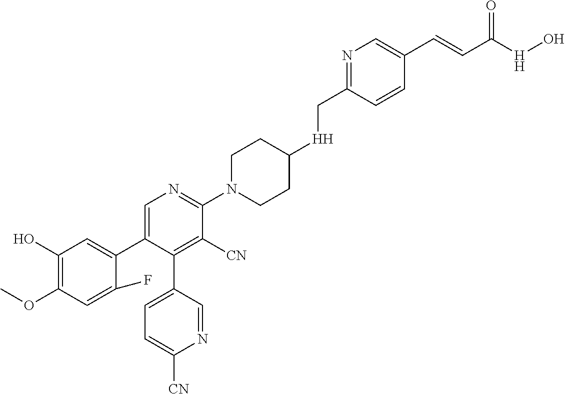 |
| 241 | (E)-3-(4-(((1-(5′- (4-Chloro-3- hydroxyphenyl)- 3′,6-dicyano- [3,4′-bipyridin]- 2′-yl)piperidin-4- yl)amino)methyl)- 3-fluorophenyl)- N- hydroxyacrylamide formate | 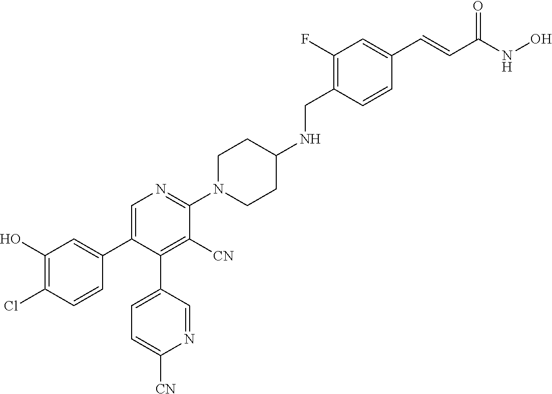 [SMILES: N#Cc1ccc(-c2c(-c3ccc(Cl)c(O)c3)cnc(N3CCC(NCc4ccc(/C=C/C(=O)NO)cc4F)CC3)c2C#N)cn1] |
| 242 | (E)-3-(4-(((1-(3- Cyano-4-(4- cyano-3- fluorophenyl)-5- (3-hydroxy-4- methoxyphenyl) pyridin-2-yl)-3- methylpiperidin- 4- yl)amino)methyl)- N- hydroxyacrylamide formate | 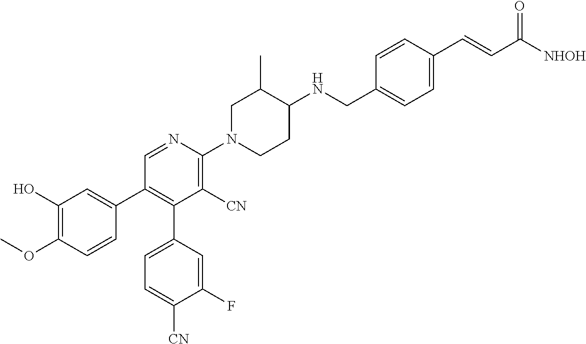 |
| 243 | (E)-3-(4-(((1- (3′,6-Dicyano-5′- (3-hydroxy-4- methoxyphenyl)- 5-methoxy-[3,4′- bipyridin]-2′- yl)piperidin-4- yl)amino)methyl) phenyl)-N- hydroxyacrylamide hydrochloride | 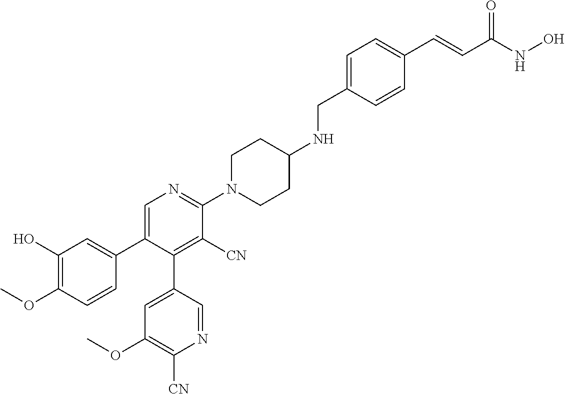 [SMILES: COc1ccc(-c2cnc(N3CCC(NCc4ccc(/C=C/C(=O)NO)cc4)CC3)c(C#N)c2-c2cnc(C#N)c(OC)c2)cc1O] |
| 244 | (E)-3-(4-(((1- (3′,6-Dicyano-5′- (3-hydroxy-4- morpholinophenyl)- [3,4′- bipyridin]-2′- yl)piperidin-4- yl)amino)methyl) phenyl)-N- hydroxyacrylamide formate | 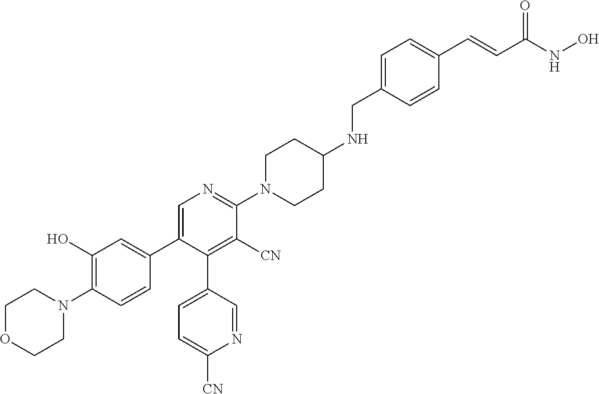 [SMILES: N#Cc1ccc(-c2c(-c3ccc(N4CCOCC4)c(O)c3)cnc(N3CCC(NCc4ccc(/C=C/C(=O)NO)cc4)CC3)c2C#N)cn1] |
| 245 | (E)-3-(4-(((1-(3′- Cyano-5′-(3- hydroxy-4- methoxyphenyl)- 6- (trifluoromethyl)- [3,4′-bipyridin]- 2′-yl)piperidin-4- yl)amino)methyl) phenyl)-N- hydroxyacrylamide formate | 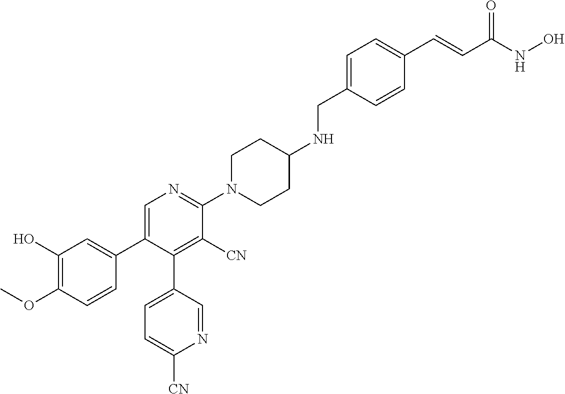 [SMILES: COc1ccc(-c2cnc(N3CCC(NCc4ccc(/C=C/C(=O)NO)cc4)CC3)c(C#N)c2-c2ccc(C#N)nc2)cc1O] |
| 246 | (E)-3-(6-(((1-(3- Cyano-4-(4- cyano-3- fluorophenyl)-5- (3-hydroxy-4- morpholinophenyl) pyridin-2- yl)piperidin-4- yl)amino)methyl) pyridin-3-yl)-N- hydroxyacrylamide formate | 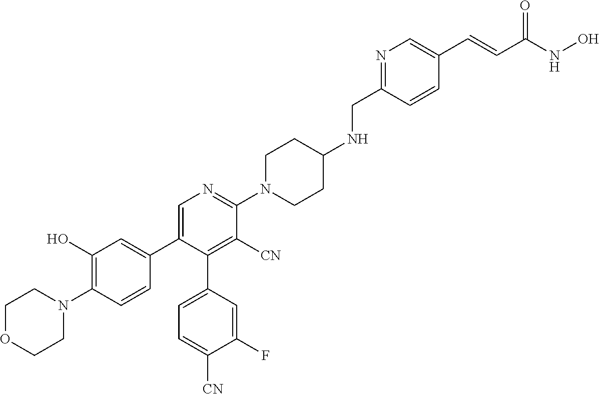 [SMILES: N#Cc1ccc(-c2c(-c3ccc(N4CCOCC4)c(O)c3)cnc(N3CCC(NCc4ccc(/C=C/C(=O)NO)cn4)CC3)c2C#N)cc1F] |
| 247 | (E)-3-(4-(((1- (3′,6-Dicyano-5′- (3-hydroxy-4- methoxyphenyl)- 5-methyl-[3,4′- bipyridin]-2′- yl)piperidin-4- yl)amino)methyl) phenyl)-N- hydroxyacrylamide formate | 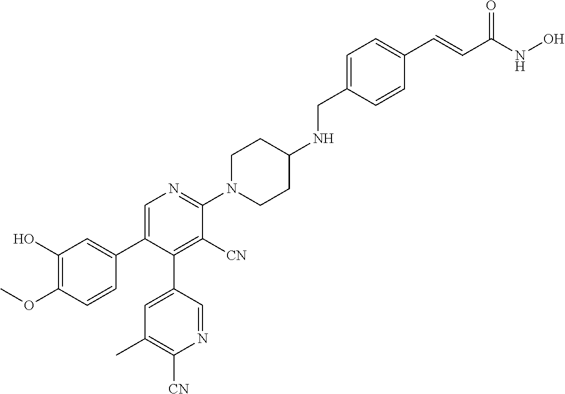 [SMILES: COc1ccc(-c2cnc(N3CCC(NCc4ccc(/C=C/C(=O)NO)cc4)CC3)c(C#N)c2-c2cnc(C#N)c(C)c2)cc1O] |
| 248 | (E)-3-(4-(((1-(3′- Cyano-5′-(3- hydroxy-4- methoxyphenyl)- 6-nitro-[3,4′- bipyridin]-2′- yl)piperidin-4- yl)amino)methyl) phenyl)-N- hydroxyacrylamide formate | 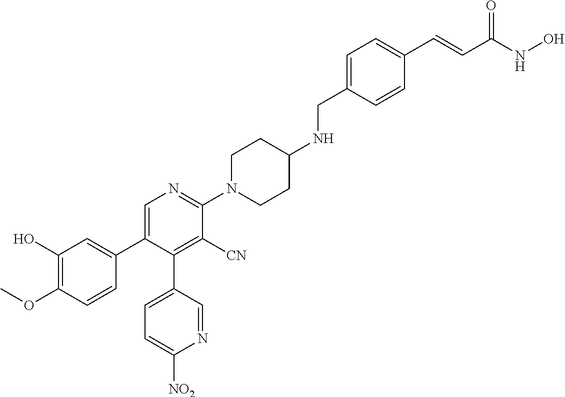 [SMILES: COc1ccc(-c2cnc(N3CCC(NCc4ccc(/C=C/C(=O)NO)cc4)CC3)c(C#N)c2-c2ccc([N+](=O)[O-])nc2)cc1O] |
| 249 | (E)-3-(4-(((1- (3′,6-Dicyano-5- fluoro-5′-(3- hydroxy-4- methoxyphenyl)- [3,4′-bipyridin]- 2′-yl)piperidin-4- yl)amino)methyl) phenyl)-N- hydroxyacrylamide formate | 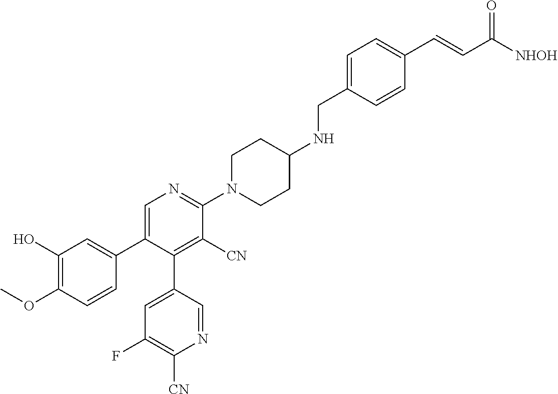 |
| 250 | E)-3-(4-(((1-(3- Cyano-4-(2- cyanopyrimidin- 5-yl)-5-(3- hydroxy-4- methoxyphenyl) pyridin-2- yl)piperidin-4- yl)amino)methyl) phenyl)-N- hydroxyacrylamide formate | 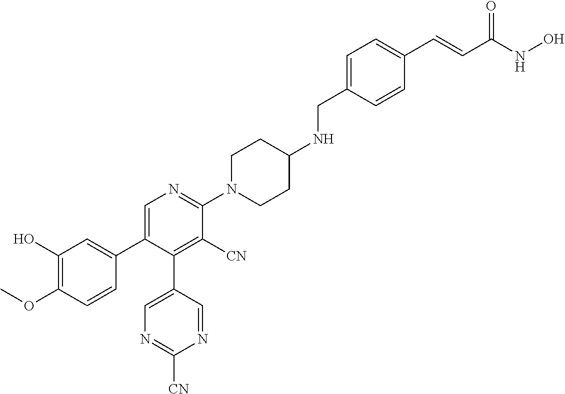 [SMILES: COc1ccc(-c2cnc(N3CCC(NCc4ccc(/C=C/C(=O)NO)cc4)CC3)c(C#N)c2-c2cnc(C#N)nc2)cc1O] |
| 251 | (E)-3-(4-(((1-(3′- Cyano-6- (difluoromethyl)- 5′-(3-hydroxy-4- methoxyphenyl)- [3,4′-bipyridin]- 2′-yl)piperidin-4- yl)amino)methyl) phenyl)-N- hydroxyacrylamide formate | 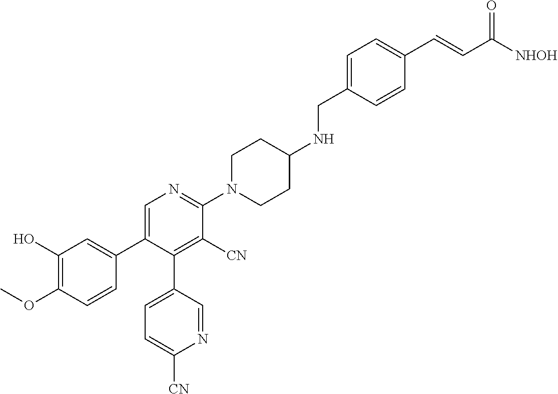 |
| 252 | (E)-3-(4-(((1- (3′,6-Dicyano-5′- (3-hydroxy-4- methoxyphenyl)- [3,4′-bipyridin]- 2′-yl)piperidin-4- yl)amino)methyl)- 3-fluorophenyl)- N- hydroxyacrylamide formate | 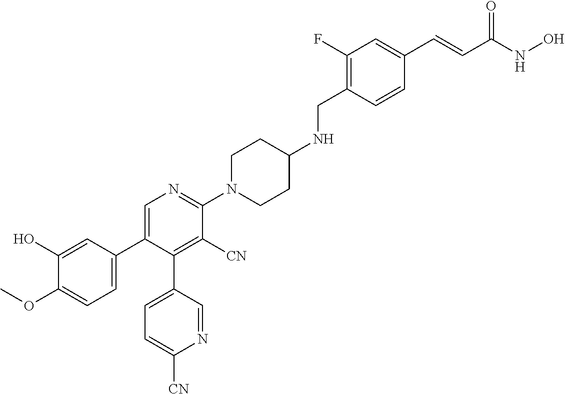 [SMILES: COc1ccc(-c2cnc(N3CCC(NCc4ccc(/C=C/C(=O)NO)cc4F)CC3)c(C#N)c2-c2ccc(C#N)nc2)cc1O] |
| 253 | Methyl (E)-3′- cyano-5′-(3- hydroxy-4- methoxyphenyl)- 2′-(4-((4-(3- (hydroxyamino)- 3-oxoprop-1-en- 1- yl)benzyl)amino) piperidin-1-yl)- [3,4′-bipyridine]- 6-carboxylate formate | 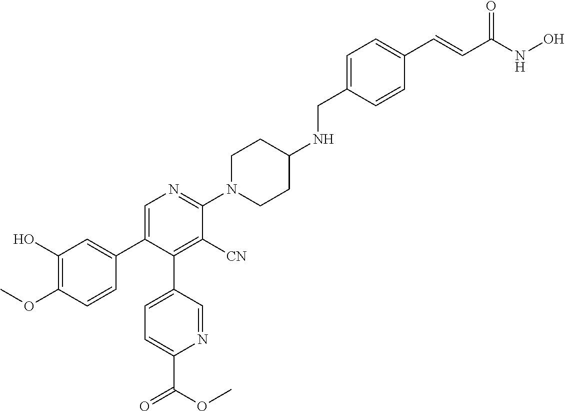 [SMILES: COC(=O)c1ccc(-c2c(-c3ccc(OC)c(O)c3)cnc(N3CCC(NCc4ccc(/C=C/C(=O)NO)cc4)CC3)c2C#N)cn1] |
| 254 | (E)-3-(4-(((1-(6- Cyano-5′-(3- hydroxy-4- methoxyphenyl)- 3′-methyl-[3,4′- bipyridin]-2′- yl)piperidin-4- yl)amino)methyl) phenyl)-N- hydroxyacrylamide formate | 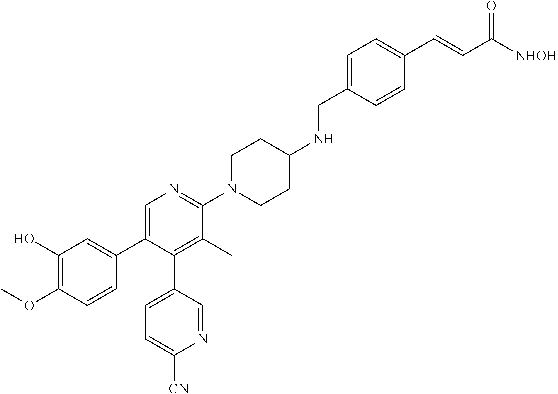 |
| 255 | (E)-3-(4-(((1- (3′,6-Dicyano-5′- (2-fluoro-5- hydroxy-4- methoxyphenyl)- [3,4′-bipyridin]- 2′-yl)piperidin-4- yl)amino)methyl) phenyl)-N- hydroxyacrylamide formate | 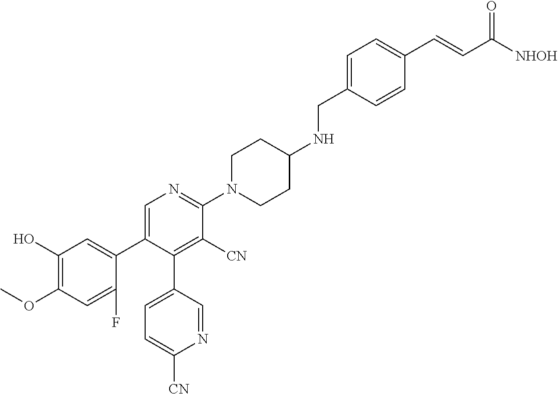 |
| 256 | (E)-3-(4-(((1-(3- Cyano-4-(5- cyanopyrazin-2- yl)-5-(3- hydroxy-4- methoxyphenyl) pyridin-2- yl)piperidin-4- yl)amino)methyl) phenyl)-N- hydroxyacrylamide formate | 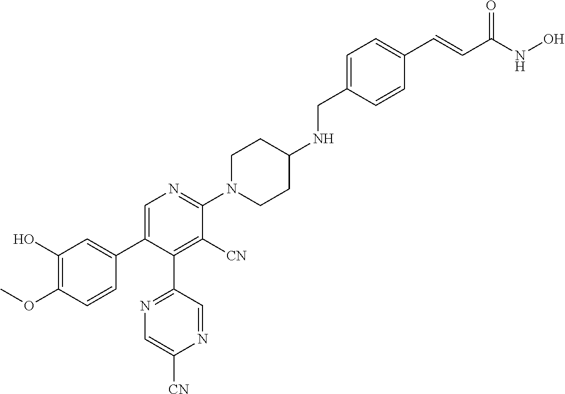 [SMILES: COc1ccc(-c2cnc(N3CCC(NCc4ccc(/C=C/C(=O)NO)cc4)CC3)c(C#N)c2-c2cnc(C#N)cn2)cc1O] |
| 257 | (E)-3-(4-(((1- (3′,6-Dicyano-5′- (3-hydroxy-4- methoxyphenyl)- [3,4′-bipyridin]- 2′-yl)piperidin-4- yl)amino)methyl)- 2- methoxyphenyl)- N- hydroxyacrylamide formate | 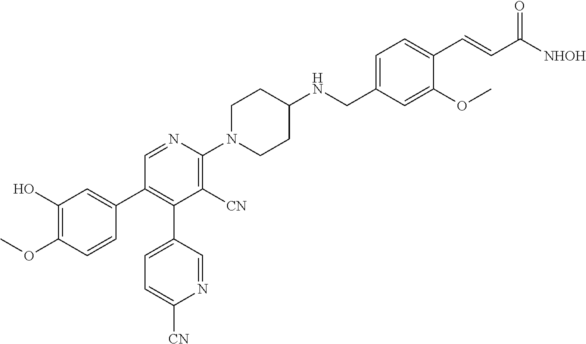 |
| 258 | (E)-3-(4-(((1- (3′,6-Dicyano-5′- (3-hydroxy-4- methoxyphenyl)- [3,4′-bipyridin]- 2′-yl)piperidin-4- yl)amino)methyl)- 2-fluorophenyl)- N- hydroxyacrylamide formate | 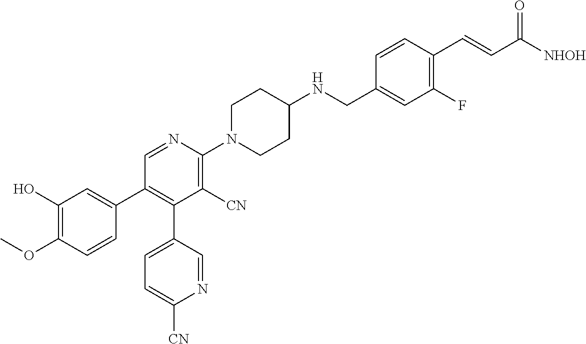 |
| 259 | (E)-3′-Cyano-5′- (3-hydroxy-4- methoxyphenyl)- 2′-(4-((4-(3- (hydroxyamino)- 3-oxoprop-1-en- 1- yl)benzyl)amino) piperidin-1-yl)- [3,4′-bipyridine]- 6-carboxamide formate | 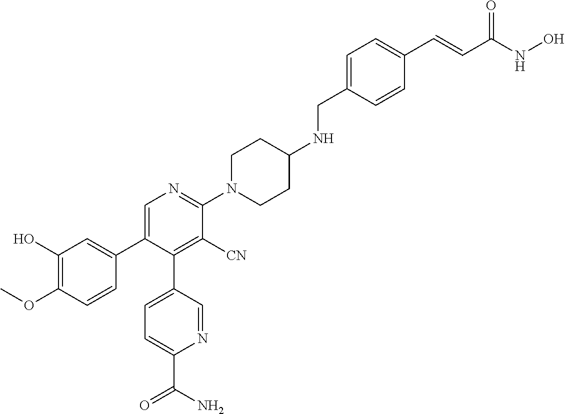 [SMILES: COc1ccc(-c2cnc(N3CCC(NCc4ccc(/C=C/C(=O)NO)cc4)CC3)c(C#N)c2-c2ccc(C(N)=O)nc2)cc1O] |
| 260 | (Z)-3-(4-(((1- (3′,6-Dicyano-5′- (3-hydroxy-4- methoxyphenyl)- [3,4′-bipyridin]- 2′-yl)piperidin-4- yl)amino)methyl) phenyl)-2-fluoro- N- hydroxyacrylamide dformate | 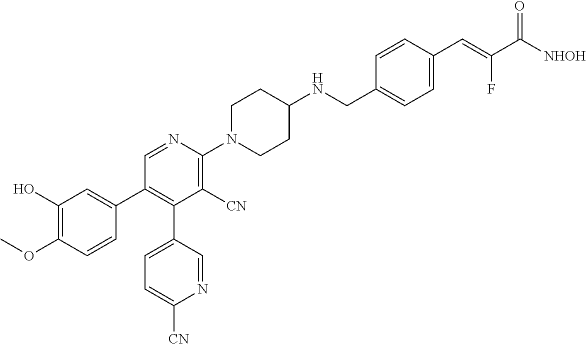 |
| 261 | (E)-3-(4-(((1-(6- Cyano-3′- (cyanomethyl)- 5′-(3-hydroxy-4- methoxyphenyl)- [3,4′-bipyridin]- 2′-yl)piperidin-4- yl)amino)methyl) phenyl)-N- hydroxyacrylamide formate | 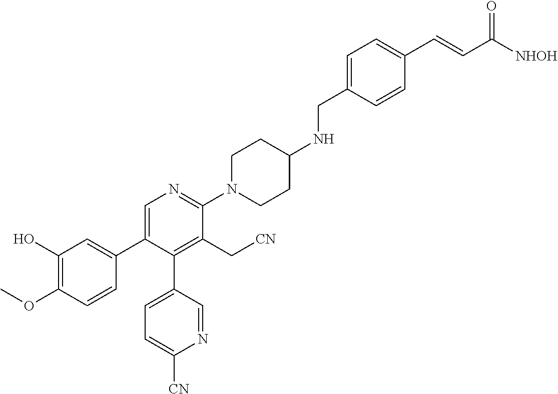 |
| 262 | (E)-3-(4-(((1-(6- Cyano-5′-(2- fluoro-5- hydroxy-4- methoxyphenyl)- 3′-methyl-[3,4′- bipyridin]-2′- yl)piperidin-4- yl)amino)methyl) phenyl)-N- hydroxyacrylamide formate | 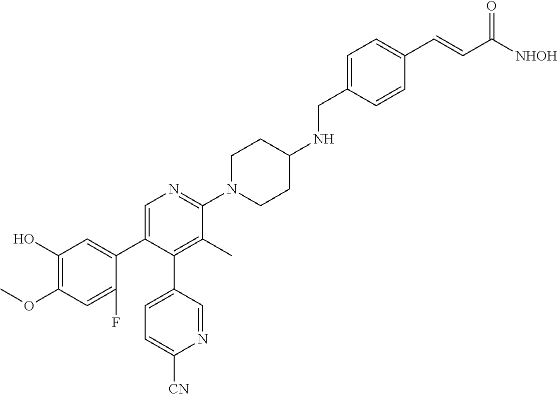 |

| Example | ¹H NMR | MS(M + H)⁺ |
|---|---|---|
| 235 | ¹H NMR (400 MHz, Methanol-d₄) δ ppm | 691.3 |
| | 8.48 (s, 1H), 8.41 (s, 1H), 7.74 (t, J = 7.2 | |
| | Hz, 1H), 7.66 (d, J = 7.8 Hz, 2H), 7.60 (d, | |
| | J = 15.8 Hz, 1H), 7.52 (d, J = 8.0 Hz, 2H), | |
| | 7.39 − 7.30 (m, 1H), 7.22 (dd, J = 8.0, 1.6 | |
| | Hz, 1H), 6.79 (d, J = 8.2 Hz, 1H), 6.58 − | |
| | 6.43 (m, 3H), 4.46 (d, J = 7.0 Hz, 1H), | |
| | 4.42 − 4.25 (m, 2H), 3.78 (s, 2H), 3.04 (d, | |
| | J = 13.2 Hz, 3H), 2.24 (d, J = 12.6 Hz, | |
| | 1H), 2.07 (d, J = 12.4 Hz, 1H), 1.81-1.67 | |
| | (m, 2H), 1.59 (d, J = 6.6 Hz, 3H), 1.30 (s, | |
| | 6H). | |
| 236 | ¹H NMR (400 MHz, DMSO-d₆) δ | 647.2 |
| | ppm: 10.82 (s, 1H), 9.10 (s, 1H), 8.72 (d, | |
| | J = 2.2 Hz, 1H), 8.53 (s, 1H), 8.00 (dd, J = | |
| | 8.1, 2.2 Hz, 1H), 7.91 (t, J = 7.4 Hz, | |
| | 1H), 7.76 (dd, J = 23.4, 7.6 Hz, 1H), 7.62 | |
| | (d, J = 8.8 Hz, 1H), 7.54 (d, J = 7.6 Hz, | |
| | 1H), 7.59 − 7.46 (m, 2H), 7.32 (d, J = | |
| | 8.1 Hz, 1H), 6.56 (d, J = 15.9 Hz, 1H), | |
| | 4.33 (d, J = 13.1 Hz, 2H), 4.03 (s, 2H), | |
| | 3.25 (t, J = 12.0 Hz, 2H), 2.93 (s, 1H), | |
| | 2.49 (t, J = 12.6 Hz, 3H), 2.06 (d, J = 12.0 | |
| | Hz, 2H), 1.54 (d, J = 11.6 Hz, 2H). | |
| 237 | ¹H NMR (400 MHz, Methanol-d₄) δ 8.52 | 606.2 |
| | (d, J = 2.0 Hz, 1H), 8.42 (s, 1H), 8.37 (s, | |
| | 1H), 7.88 − 7.76 (m, 2H), 7.57 − 7.45 (m, | |
| | 3H), 7.39 (d, J = 7.9 Hz, 2H), 7.10 (d, J = | |
| | 8.2 Hz, 1H), 6.52 (d, J = 2.2 Hz, 1H), 6.46 − | |
| | 6.36 (m, 2H), 4.39 (d, J = 13.4 Hz, 2H), | |
| | 3.97 (s, 2H), 3.15 − 3.00 (m, 3H), 2.11 (d, | |
| | J = 12.4 Hz, 2H), 1.61 (q, J = 11.6 Hz, | |
| | 2H). | |
| 238 | ¹H NMR (400 MHz, Methanol-d₄) δ 8.71 | 607.2 |
| | (s, 1H), 8.52 (s, 1H), 8.40 (s, 1H), 8.05 − | |
| | 7.95 (m, 1H), 7.88 − 7.75 (m, 2H), 7.53 | |
| | (d, J = 8.8 Hz, 2H), 7.43 (d, J = 8.2 Hz, | |
| | 1H), 7.10 (d, J = 8.1 Hz, 1H), 6.60 − 6.48 | |
| | (m, 2H), 6.42 (d, J = 8.0 Hz, 1H), 4.44 (d, | |
| | J = 17.0 Hz, 4H), 3.18 − 2.98 (m, 3H), | |
| | 2.25 (d, J = 12.2 Hz, 2H), 1.82 (d, J = 12.6 | |
| | Hz, 2H). | |
| 239 | ¹H NMR (400 MHz, Methanol-d₄) δ 8.75 | 624.2 |
| | (d, J = 2.1 Hz, 1H), 8.46 (d, J = 21.4 Hz, | |
| | 2H), 8.06 (dd, J = 8.1, 2.2 Hz, 1H), 7.76 | |
| | (t, J = 7.3 Hz, 1H), 7.61 (d, J = 15.9 Hz, | |
| | 1H), 7.53 (d, J = 8.2 Hz, 1H), 7.42 (d, J = | |
| | 9.6 Hz, 1H), 7.25 − 7.16 (m, 2H), 6.68 − | |
| | 6.56 (m, 2H), 6.53 (dd, J = 8.2, 2.0 Hz, | |
| | 1H), 4.46 (d, J = 13.3 Hz, 2H), 4.29 (s, | |
| | 2H), 3.24 (s, 1H), 3.18 (t, J = 12.9 Hz, | |
| | 2H), 2.27 − 2.20 (m, 2H), 1.79 (dd, J = | |
| | 13.4, 9.6 Hz, 2H). | |
| 240 | ¹H NMR (400 MHz, DMSO-d₆) δ 10.82 | 621.2 |
| | (s, 1H), 9.07 (s, 1H), 8.85 − 8.60 (m, 2H), | |
| | 8.42 (s, 1H), 8.14 (d, J = 1.6 Hz, 1H), 8.11 | |
| | (d, J = 8.0 Hz, 1H), 8.06 − 7.95 (m, 2H), | |
| | 7.65 − 7.44 (m, 2H), 6.67 (m, 2H), 6.57 | |
| | (d, J = 16.0 Hz, 1H), 4.29 (d, J = 13.2 Hz, | |
| | 2H), 4.09 (s, 2H), 3.71 (s, 3H), 3.19 (d, J = | |
| | 12.0 Hz, 2H), 3.00 (s, 1H), 2.08 (d, J = | |
| | 12.4 Hz, 2H), 1.57 (d, J = 11.6 Hz, 2H). | |
| 241 | ¹H NMR (400 MHz, DMSO-d₆) δ 10.80 | 624.2 |
| | (s, 1H), 10.27 (s, 1H), 9.12 (s, 1H), 8.72 | |
| | (d, J = 2.0 Hz, 1H), 8.52 (s, 1H), 8.15 (d, | |
| | J = 8.0 Hz, 1H), 8.05 (dd, J = 8.0, 2.2 Hz, | |
| | 1H), 7.68 (t, J = 7.6 Hz, 1H), 7.61 − 7.42 | |
| | (m, 3H), 7.24 (d, J = 8.2 Hz, 1H), 6.66 − | |
| | 6.46 (m, 3H), 4.40 (s, 2H), 4.36 (t, J = 9.2 | |
| | Hz, 2H), 4.27 (s, 2H), 3.50 (s, 1H), 3.18 | |
| | (t, J = 12.0 Hz, 2H), 2.26 (s, 2H). | |
| 242 | ¹H NMR (400 MHz, Methanol-d₄) δ ppm | 633.3 |
| | 8.51 (s, 1H), 8.40 (s, 1H), 7.74 (t, J = 7.2 | |
| | Hz, 1H), 7.67-7.53 (m, 3H), 7.47 (d, J = | |
| | 7.8 Hz, 2H), 7.36 (d, J = 9.6 Hz, 1H), 7.23 | |
| | (d, J = 8.0 Hz, 1H), 6.81 (d, J = 8.6 Hz, | |
| | 1H), 6.56-6.42 (m, 3H), 4.41 (d, J = 13.2 | |
| | Hz, 1H), 4.28 (d, J = 12.8 Hz, 1H), 4.08 | |
| | (d, J = 13.4 Hz, 1H), 3.93 (d, J = 13.4Hz, | |
| | 1H), 3.81 (s, 3H), 3.18-3.09 (m, 1H), 2.84 | |
| | (dd, J = 13.4, 10.6 Hz, 1H), 2.63 (s, 1H), | |
| | 2.22 (d, J = 13.2 Hz, 1H), 1.91 (s, 1H), | |
| | 1.68 (d, J = 11.8 Hz, 1H), 1.09 (d, J = 6.6 | |
| | Hz, 3H). | |
| 243 | ¹H NMR (400 MHz, DMSO-d₆) δ 10.82 | 632.3 |
| | (s, 1H), 9.42 (s, 2H), 8.50 (s, 1H), 8.42 (s, | |
| | 1H), 8.12 (d, J = 1.6 Hz, 1H), 7.96 (d, J = | |
| | 1.6 Hz, 1H), 7.64 (s, 3H), 7.48 (d, J = 15.6 | |
| | Hz, 1H), 6.84 (d, J = 8.0 Hz, 1H), 6.59 − | |
| | 6.43 (m, 3H), 4.35 (d, J = 13.2 Hz, 2H), | |
| | 4.24 (t, J = 6.0 Hz, 2H), 3.89 (s, 3H), 3.73 | |
| | (s, 3H), 3.37 (d, J = 13.6 Hz, 1H), 3.14 (t, | |
| | J = 12.8 Hz, 2H), 2.29 (d, J = 12.0 Hz, | |
| | 2H), 1.84 (t, J = 12.0 Hz, 2H). | |
| 244 | ¹H NMR (400 MHz, DMSO-d₆) δ 10.80 | 657.3 |
| | (s, 1H), 9.22 (s, 1H), 9.08 (s, 1H), 8.68 (d, | |
| | J = 2.2 Hz, 1H), 8.49 (s, 1H), 8.18 − 8.10 | |
| | (m, 1H), 8.06 (dd, J = 8.0, 2.2 Hz, 1H), | |
| | 7.63 (d, J = 7.6 Hz, 2H), 7.59 − 7.41 (m, | |
| | 3H), 6.75 (d, J = 8.2 Hz, 1H), 6.56 − 6.45 | |
| | (m, 2H), 6.41 (d, J = 2.0 Hz, 1H), 4.31 (d, | |
| | J = 12.8 Hz, 2H), 4.19 (s, 2H), 3.69 (t, J = | |
| | 4.4 Hz, 4H), 3.30 (s, 1H), 3.15 (t, J = | |
| | 12.4 Hz, 2H), 2.91 (d, J = 4.4 Hz, 4H), | |
| | 2.22 (s, 2H), 1.71 (s, 2H). | |
| 245 | ¹H NMR (400 MHz, DMSO-d₆) δ ppm: | 645.2 |
| | 10.75 (s, 1H), 9.01 (s, 1H), 8.67 (d, J = | |
| | 2.0 Hz, 1H), 8.45 (s, 1H), 8.16 (s, 1H), | |
| | 8.07 (dd, J = 8.1, 2.1 Hz, 1H), 7.98 (d, J = | |
| | 8.1 Hz, 1H), 7.55 (d, J = 7.9 Hz, 2H), 7.51 − | |
| | 7.41 (m, 3H), 6.81 (d, J = 8.1 Hz, 1H), | |
| | 6.51 − 6.42 (m, 3H), 4.24 (d, J = 13.1 Hz, | |
| | 2H), 3.94 (s, 2H), 3.71 (s, 3H), 3.17 (t, J = | |
| | 12.0 Hz, 2H), 2.93 (s, 1H), 2.08 (d, J = | |
| | 12.2 Hz, 2H), 1.56 (q, J = 11.5, 10.9 Hz, | |
| | 2H). | |
| 246 | ¹H NMR (400 MHz, Methanol-d₄) δ 8.75 | 675.3 |
| | (d, J = 2.2 Hz, 1H), 8.50 (s, 1H), 8.42 (s, | |
| | 1H), 8.05 (dd, J = 8.2, 2.2 Hz, 1H), 7.78 − | |
| | 7.70 (m, 1H), 7.61 (d, J = 15.9 Hz, 1H), | |
| | 7.53 (d, J = 8.1 Hz, 1H), 7.36 (dd, J = 9.7, | |
| | 1.4 Hz, 1H), 7.23 (dd, J = 8.0, 1.5 Hz, | |
| | 1H), 6.89 (d, J = 8.1 Hz, 1H), 6.67 − 6.47 | |
| | (m, 3H), 4.42 (d, J = 13.4 Hz, 2H), 4.29 | |
| | (s, 2H), 3.81 (t, J = 4.5 Hz, 4H), 3.19 (q, | |
| | J = 14.4, 12.8 Hz, 3H), 2.94 (t, J = 4.5 Hz, | |
| | 4H), 2.23 (d, J = 12.3 Hz, 2H), 1.87 − 1.70 | |
| | (m, 2H). | |
| 247 | ¹H NMR (400 MHz, DMSO-d₆) δ ppm: | 616.3 |
| | 10.74 (s, 1H), 8.82 (d, J = 124.3 Hz, 1H), | |
| | 8.44 (s, 1H), 8.40 (d, J = 1.9 Hz, 1H), 8.18 | |
| | (d, J = 1.3 Hz, 1H), 8.02 (d, J = 2.0 Hz, | |
| | 1H), 7.54 (d, J = 7.9 Hz, 2H), 7.49 − 7.40 | |
| | (m, 3H), 6.81 (d, J = 8.1 Hz, 1H), 6.50 − | |
| | 6.41 (m, 3H), 4.23 (dd, J = 10.4, 6.4 Hz, | |
| | 2H), 3.91 (s, 2H), 3.71 (s, 3H), 3.15 (d, J = | |
| | 11.2 Hz, 2H), 2.94 − 2.84 (m, 1H), 2.53 − | |
| | 2.46 (m, 3H), 2.05 (dd, J = 12.7, 3.9 Hz, | |
| | 2H), 1.53 (q, J = 10.3 Hz, 2H). | |
| 248 | ¹H NMR (400 MHz, Methanol-d₄) δ ppm | 622.2 |
| | 8.50 (d, J = 5.8 Hz, 2H), 8.44 (s, 1H), | |
| | 8.32 (d, J = 8.2 Hz, 1H), 8.14 (dd, J = 8.4, | |
| | 2.2 Hz, 1H), 7.73-7.45 (m, 5H), 6.94-6.70 | |
| | (m, 1H), 6.59- 6.27 (m, 3H), 4.49 (d, J = | |
| | 13.4 Hz, 2H), 4.25 (s, 2H), 3.80 (s, 3H), | |
| | 3.42 (d, J = 12.0 Hz, 1H), 3.20 (t, J = 12.8 | |
| | Hz, 2H), 2.31 (d, J = 12.2 Hz, 2H), 1.96- | |
| | 1.70 (m, 2H). | |
| 249 | ¹H NMR (400 MHz, DMSO-d₆) δ 10.73 | 620.2 |
| | (s, 1H), 9.07 (s, 1H), 8.48 (s, 2H), 8.35 | |
| | (dd, J = 9.6, 1.6 Hz, 1H), 8.15 (s, 1H), | |
| | 7.54 (d, J = 7.8 Hz, 2H), 7.48 − 7.40 (m, | |
| | 3H), 6.83 (d, J = 8.4 Hz, 1H), 6.52 − 6.40 | |
| | (m, 3H), 4.23 (d, J = 13.2 Hz, 2H), 3.89 | |
| | (s, 2H), 3.72 (s, 3H), 3.19 (t, J = 12.0 Hz, | |
| | 2H), 2.86 (s, 1H), 2.04 (d, J = 12.4 Hz, | |
| | 2H), 1.51 (q, J = 11.2 Hz, 2H). | |
| 250 | ¹H NMR (400 MHz, Methanol-d₄) δ 8.85 | 603.2 |
| | (s, 2H), 8.49 (s, 1H), 8.48 (s, 1H), 7.70 − | |
| | 7.58 (m, 2H), 7.54 (t, J = 8.7 Hz, 3H), | |
| | 6.86 (d, J = 8.1 Hz, 1H), 6.53 (dd, J = 4.0, | |
| | 1.8 Hz, 2H), 6.50 (d, J = 2.1 Hz, 1H), 4.50 | |
| | (d, J = 13.4 Hz, 2H), 4.21 (s, 2H), 3.83 (s, | |
| | 3H), 3.37 (d, J = 11.7 Hz, 1H), 3.20 (t, J = | |
| | 12.7 Hz, 2H), 2.29 (d, J = 12.2 Hz, 2H), | |
| | 1.81 (tt, J = 12.6, 5.8 Hz, 2H). | |
| 251 | ¹H NMR (400 MHz, DMSO-d₆) δ 10.74 | 627.3 |
| | (s, 1H), 8.99 (s, 1H), 8.51 (s, 1H), 8.55 (d, | |
| | J = 2.1 Hz, 1H), 8.42 (s, 1H), 7.94 (dd, J = | |
| | 8.2, 2.2 Hz, 1H), 7.72 (d, J = 8.1 Hz, | |
| | 1H), 7.55 (d, J = 7.9 Hz, 2H), 7.49 − 7.40 | |
| | (m, 3H), 6.86 − 6.76 (m, 1H), 6.45 (ddd, | |
| | J = 7.8, 6.0, 3.4 Hz, 3H), 4.22 (d, J = 13.0 | |
| | Hz, 2H), 3.95 (s, 2H), 3.69 (s, 3H), 3.15 | |
| | (t, J = 12.2 Hz, 2H), 2.94 (s, 1H), 2.15 − | |
| | 1.99 (m, 2H), 1.55 (d, J = 11.7 Hz, 2H). | |
| 252 | ¹H NMR (400 MHz, DMSO-d₆) δ ppm | 620.2 |
| | 10.81 (s, 1H), 9.03 (s, 2H), 8.65 (s, 1H), | |
| | 8.46 (s, 1H), 8.13 (d, J = 7.8 Hz, 1H), 8.06 | |
| | (dd, J = 8.0, 1.2 Hz, 1H), 7.60 (t, J = 13.6 | |
| | Hz, 1H), 7.52 − 7.37 (m, 3H), 6.79 (dd, | |
| | J = 19.2, 14.2 Hz, 1H), 6.53 (d, J = 15.8 Hz, | |
| | 1H), 6.45 (d, J = 6.0 Hz, 2H), 4.26 (d, J = | |
| | 12.8 Hz, 2H), 4.04 (s, 2H), 3.71 (s, 3H), | |
| | 3.15 (dd, J = 29.8, 18.0 Hz, 3H), 2.13 (d, | |
| | J = 10.2 Hz, 2H), 1.61 (d, J = 10.2 Hz, | |
| | 2H) | |
| 253 | ¹H NMR (400 MHz, DMSO-d₆) δ 10.73 | 635.3 |
| | (s, 1H), 8.98 (s, 1H), 8.57 (d, J = 2.0 Hz, | |
| | 1H), 8.43 (s, 1H), 8.25 (s, 1H), 8.06 (d, | |
| | J = 8.0 Hz, 1H), 7.95 (dd, J = 8.0, 2.2 Hz, | |
| | 1H), 7.53 (d, J = 8.0 Hz, 2H), 7.50 − 7.38 | |
| | (m, 3H), 6.79 (d, J = 8.1 Hz, 1H), 6.50 − | |
| | 6.40 (m, 3H), 4.21 (d, J = 13.0 Hz, 2H), | |
| | 3.88 (s, 5H), 3.70 (s, 3H), 3.17 (t, J = 12.0 | |
| | Hz, 2H), 2.84 (s, 1H), 2.03 (d, J = 12.4 | |
| | Hz, 2H), 1.53 (dd, J = 7.0, 9.2 Hz, 2H). | |
| 254 | ¹H NMR (400 MHz, DMSO-d₆) δ 10.95 | 591.3 |
| | (s, 1H), 9.07 (s, 1H), 8.94 (s, 1H), 8.48 (d, | |
| | J = 2.0 Hz, 1H), 8.16 (s, 1H), 8.05 (d, J = | |
| | 8.0 Hz, 1H), 7.85 (dd, J = 8.0, 2.0 Hz, | |
| | 1H), 7.62 (q, J = 8.0 Hz, 4H), 7.48 (d, J = | |
| | 15.9 Hz, 1H), 6.76 (d, J = 8.2 Hz, 1H), | |
| | 6.52 (d, J = 15.8 Hz, 1H), 6.46 − 6.35 (m, | |
| | 2H), 4.22 (s, 2H), 3.70 (d, J = 5.2 Hz, | |
| | 3H), 3.64 − 3.52 (m, 2H), 3.20 (s, 1H), | |
| | 2.85 (t, J = 12.2 Hz, 2H), 2.21 (d, J = 11.6 | |
| | Hz, 2H), 2.00 (s, 3H), 1.81 (d, J = 11.6 | |
| | Hz, 2H). | |
| 255 | ¹H NMR (400 MHz, Methanol-d₄) δ 8.59 | 620.2 |
| | (s, 1H), 8.44 (s, 1H), 8.25 (s, 1H), 7.92 | |
| | (dd, J = 8.0, 2.2 Hz, 1H), 7.89 − 7.83 (m, | |
| | 1H), 7.68 (d, J = 8.0 Hz, 2H), 7.64 − 7.52 | |
| | (m, 3H), 6.69 − 6.48 (m, 3H), 4.58 − 4.50 | |
| | (m, 2H), 4.33 (s, 2H), 3.80 (s, 3H), 3.54 | |
| | (s, 1H), 3.21 (t, J = 12.8 Hz, 2H), 2.35 (d, | |
| | J = 12.0 Hz, 2H), 1.88 (tt, J = 12.8, 7.2 | |
| | Hz, 2H). | |
| 256 | ¹H NMR (400 MHz, DMSO-d₆) δ 10.72 | 603.2 |
| | (s, 1H), 9.41 (d, J = 1.6 Hz, 1H), 9.09 (s, | |
| | 1H), 8.71 (d, J = 1.6 Hz, 1H), 8.55 (s, | |
| | 1H), 8.15 (s, 1H), 7.53 (d, J = 7.6 Hz, | |
| | 2H), 7.44 (t, J = 8.4 Hz, 3H), 6.82 (d, J = | |
| | 8.4 Hz, 1H), 6.48 − 6.35 (m, 3H), 4.21 | |
| | (d, J = 13.2 Hz, 2H), 3.86 (s, 2H), 3.72 | |
| | (s, 3H), 3.21 (t, J = 12.1 Hz, 2H), 2.81 | |
| | (s, 1H), 2.02 (d, J = 11.6 Hz, 2H), 1.49 | |
| | (q, J = 10.8, 10.2 Hz, 2H). | |
| 257 | ¹H NMR (400 MHz, Methanol-d₄) δ ppm | 632.3 |
| | 8.62 (s, 1H),8.58 (dd, J = 2.2, 0.9 Hz, | |
| | 1H), 8.48 (s, 1H), 7.98-7.78 (m, 3H), 7.62 | |
| | (d, J = 7.8 Hz, 1H), 7.28-7.21 (m, 1H), | |
| | 7.13 (d, J = 7.8 Hz, 1H), 6.83 (d, J = 8.0 | |
| | Hz, 1H), 6.62 (d, J = 16.0 Hz, 1H), 6.54- | |
| | 6.45 (m, 2H), 4.50 (d, J = 13.4 Hz, 2H), | |
| | 4.33 (s, 2H), 3.98 (s, 3H), 3.81 (s, 3H), | |
| | 3.54 (t, J = 12.0 Hz, 1H), 3.21 (t, J = 12.8 | |
| | Hz, 2H), 2.36 (d, J = 11.4 Hz, 2H), 1.90 | |
| | (d, J = 11.4 Hz, 2H). | |
| 258 | ¹H NMR (400 MHz, DMSO-d₆) δ 10.89 | 620.2 |
| | (s, 1H), 9.12 (s, 1H), 9.04 (s, 1H), 8.65 (d, | |
| | J = 2.2 Hz, 1H), 8.47 (s, 1H), 8.13 (d, J = | |
| | 8.0 Hz, 1H), 8.05 (dd, J = 8.2, 2.2 Hz, | |
| | 1H), 7.72 (t, J = 8.1 Hz, 1H), 7.52 (t, J = | |
| | 13.6 Hz, 2H), 7.43 (d, J = 7.8 Hz, 1H), | |
| | 6.80 (d, J = 8.9 Hz, 1H), 6.61 (d, J = 16.0 | |
| | Hz, 1H), 6.47 − 6.40 (m, 2H), 4.31 (d, J = | |
| | 13.2 Hz, 2H), 4.23 (s, 2H), 3.71 (s, 3H), | |
| | 3.54 (t, J = 11.0 Hz, 1H), 3.14 (t, J = 12.6 | |
| | Hz, 2H), 2.22 (s, 2H), 1.75 (s, 2H). | |
| 259 | ¹H NMR (400 MHz, DMSO-d₆) δ 10.85 | 620.3 |
| | (s, 1H), 9.51 (s, 2H), 9.10 (s, 1H), 8.49 (d, | |
| | J = 2.2 Hz, 1H), 8.46 (s, 1H), 8.18 (d, J = | |
| | 2.4 Hz, 1H), 8.04 (d, J = 8.0 Hz, 1H), 7.95 | |
| | (dd, J = 8.0, 2.2 Hz, 1H), 7.72 (s, 1H), | |
| | 7.69 − 7.57 (m, 4H), 7.48 (d, J = 15.8 Hz, | |
| | 1H), 6.80 (d, J = 8.2 Hz, 1H), 6.59 − 6.48 | |
| | (m, 2H), 6.45 (dd, J = 8.2, 2.2 Hz, 1H), | |
| | 4.34 (d, J = 12.8 Hz, 2H), 4.23 (d, J = 6.2 | |
| | Hz, 2H), 3.70 (s, 3H), 3.37 (s, 1H), 3.13 | |
| | (t, J = 12.6 Hz, 2H), 2.29 (d, J = 11.8 Hz, | |
| | 2H), 1.95 − 1.76 (m, 2H). | |
| 260 | ¹H NMR (400 MHz, Methanol-d₄) δ ppm | 620.2 |
| | 8.57 (d, J = 2.0 Hz, 1H), 8.50 (s, 1H),8.46 | |
| | (d, J = 2.4 Hz, 1H), 7.98-7.84 (m, 2H), | |
| | 7.71 (d, J = 8.2 Hz, 1H), 7.62 (d, J = 8.0 | |
| | Hz, 1H), 7.53 (d, J = 8.0 Hz, 1H), 7.45 (d, | |
| | J = 8.0 Hz, 1H), 6.82 (dd, J = 8.2 Hz, 2H), | |
| | 6.55-6.45 (m, 2H), 4.50- 4.42 (m, 2H), | |
| | 4.18 (d, J = 7.4 Hz, 2H),3.28 (s, 1H), 3.81 | |
| | (s, 3H), 3.24-3.14 (m, 2H), 2.27 (s, 2H), | |
| | 1.86-1.73 (m, 2H). | |
| 261 | ¹H NMR (400 MHz, DMSO-d₆) δ 10.72 | 616.3 |
| | (s, 1H), 8.97 (s, 1H), 8.52 (d, J = 2.0 Hz, | |
| | 1H), 8.33 (s, 1H), 8.24 (s, 1H), 8.11 (d, | |
| | J = 8.0 Hz, 1H), 7.95 (dd, J = 8.0, 2.0 Hz, | |
| | 1H), 7.51 (d, J = 7.6 Hz, 2H), 7.43 (t, J = | |
| | 10.4 Hz, 3H), 6.77 (d, J = 8.4 Hz, 1H), | |
| | 6.43 (d, J = 10.0 Hz, 3H), 3.80 (d, J = | |
| | 5.6 Hz, 4H), 3.70 (s, 3H), 3.36 (s, 2H), | |
| | 2.88 (t, J = 11.6 Hz, 2H), 2.64 (t, J = | |
| | 12.4 Hz, 1H), 1.99 (d, J = 12.4 Hz, 2H), | |
| | 1.56 (d, J = 11.6 Hz, 2H). | |
| 262 | ¹H NMR (400 MHz, DMSO-d₆) δ 10.72 | 609.3 |
| | (s, 1H), 8.98 (s, 1H), 8.48 (d, J = 2.0 Hz, | |
| | 1H), 8.28 (s, 1H), 8.10 (s, 1H), 8.02 (d, J = | |
| | 8.0 Hz, 1H), 7.82 (dd, J = 8.0, 2.2 Hz, | |
| | 1H), 7.52 (d, J = 7.8 Hz, 2H), 7.48 − 7.36 | |
| | (m, 3H), 6.65 (d, J = 11.4 Hz, 1H), 6.57 | |
| | (d, J = 7.6 Hz, 1H), 6.44 (d, J = 15.8 Hz, | |
| | 1H), 3.83 (s, 2H), 3.70 (s, 3H), 3.51 − | |
| | 3.44 (m, 2H), 2.82 (t, J = 11.8 Hz, 2H), | |
| | 2.64 (d, J = 9.6 Hz, 1H), 2.00-1.80 (m, | |
| | 5H), 1.49 (q, J = 11.0 Hz, 2H). | |

Examples 263-266 were prepared similarly according to the synthetic method of Example 88, and the structure and characterization data are as follows.

| Example | Chemical name | Structure | | |
|---|---|---|---|---|
| 263 | (E)-3-(4-(((1-(6- Cyano-5′-(3- hydroxy-4- methoxyphenyl)-3′- methoxy-[3,4′- bipyridin]-2′- yl)piperidin-4- yl)amino)methyl) phenyl)-N- hydroxyacrylamide formate | 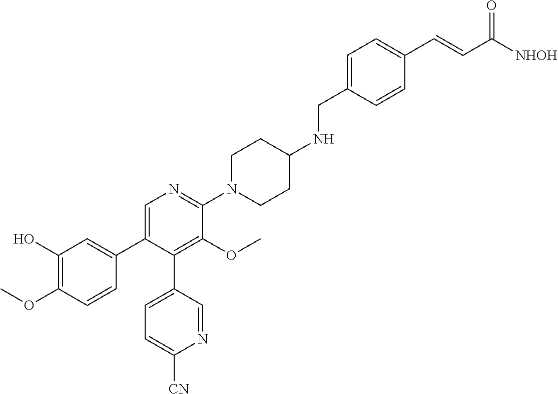 | | |
| 264 | (E)-3-(4-(((1-(6- Cyano-5′-(3- hydroxy-4- methoxyphenyl)-3′- methoxy-[3,4′- bipyridin]-2′- yl)piperidin-4- yl)amino)methyl)-3- fluorophenyl)-N- hydroxyacrylamide formate | 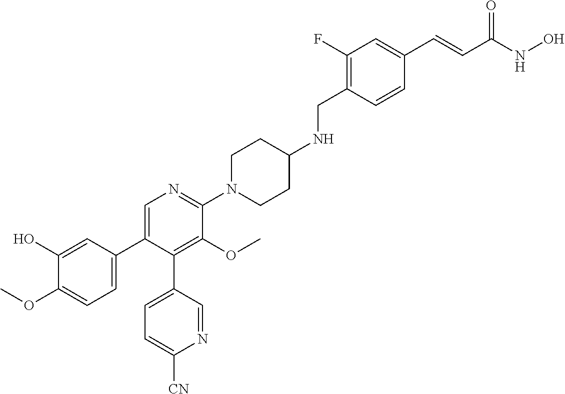 [SMILES: COc1ccc(-c2cnc(N3CCC(NCc4ccc(/C=C/C(=O)NO)cc4F)CC3)c(OC)c2-c2ccc(C#N)nc2)cc1O] | | |
| 265 | (E)-3-(4-(((1-(6- Cyano-5′-(2-fluoro- 5-hydroxy-4- methoxyphenyl)-3′- methoxy-[3,4′- bipyridin]-2′- yl)piperidin-4- yl)amino)methyl) phenyl)-N- hydroxyacrylamide formate | 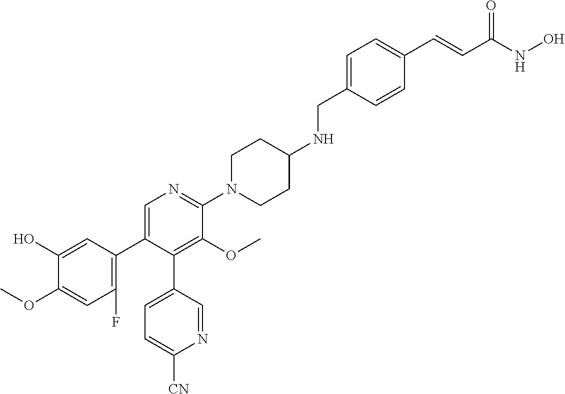 [SMILES: COc1cc(F)c(-c2cnc(N3CCC(NCc4ccc(/C=C/C(=O)NO)cc4)CC3)c(OC)c2-c2ccc(C#N)nc2)cc1O] | | |
| 266 | (E)-3-(5-(((1-(6- Cyano-5′-(3- hydroxy-4- methoxyphenyl)-3′- methoxy-[3,4′- bipyridin]-2′- yl)piperidin-4- yl)amino)methyl) pyridin-2-yl)-N- hydroxyacrylamide formate | 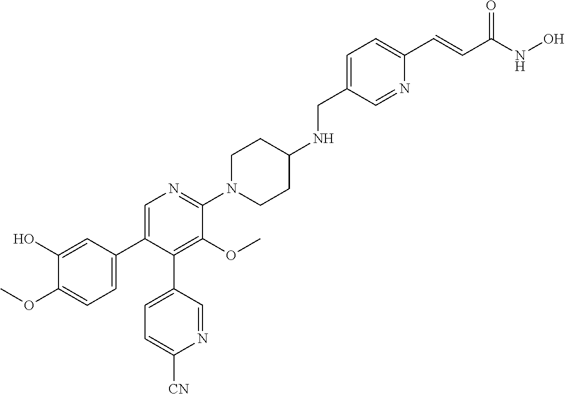 [SMILES: COc1ccc(-c2cnc(N3CCC(NCc4ccc(/C=C/C(=O)NO)nc4)CC3)c(OC)c2-c2ccc(C#N)nc2)cc1O] | | |
| | | Example | ¹H NMR | MS(M + H)⁺ |
| | | 263 | ¹H NMR (400 MHz, DMSO-d₆) δ 10.74 | 607.3 |
| | | | (s, 1H), 8.95 (s, 1H), 8.56 − 8.41 (m, 1H), | |
| | | | 8.16 (s, 1H), 8.08 − 7.94 (m, 2H), 7.83 | |
| | | | (dd, J = 8.0, 2.0 Hz, 1H), 7.55 (d, J = 8.0 | |
| | | | Hz, 2H), 7.50 − 7.39 (m, 3H), 6.78 (d, J = | |
| | | | 8.0 Hz, 1H), 6.52 − 6.36 (m, 3H), 4.05 (d, | |
| | | | J = 12.8 Hz, 2H), 3.93 (s, 2H), 3.71 (s, | |
| | | | 3H), 3.45 (s, 3H), 2.88 (t, J = 12.4 Hz, | |
| | | | 3H), 2.04 (d, J = 12.0 Hz, 2H), 1.55 (q, | |
| | | | J = 11.2 Hz, 2H). | |
| | | 264 | ¹H NMR (400 MHz, DMSO-d₆) δ 10.80 | 625.3 |
| | | | (s, 1H), 9.09 (s, 1H), 8.95 (s, 1H), 8.50 (d, | |
| | | | J = 2.0 Hz, 1H), 8.13 (s, 1H), 8.02 (d, J = | |
| | | | 8.4 Hz, 2H), 7.84 (dd, J = 8.0, 2.0 Hz, | |
| | | | 1H), 7.60 (s, 1H), 7.45 (d, J = 12.0 Hz, | |
| | | | 2H), 6.78 (d, J = 8.0 Hz, 1H), 6.52 (d, J = | |
| | | | 15.6 Hz, 1H), 6.46 − 6.35 (m, 2H), 4.09 | |
| | | | (d, J = 12.4 Hz, 4H), 3.71 (s, 3H), 3.45 (s, | |
| | | | 3H), 3.36 (d, J = 11.2 Hz, 1H), 2.90 (t, | |
| | | | J = 12.4 Hz, 2H), 2.10 (s, 2H), 1.63 (s, 2H). | |
| | | 265 | ¹H NMR (400 MHz, DMSO-d₆) δ 10.79 | 625.2 |
| | | | (s, 1H), 9.03 (s, 1H), 8.51 (d, J = 2.0 Hz, | |
| | | | 1H), 8.25 (s, 1H), 8.02 (t, J = 4.0 Hz, 2H), | |
| | | | 7.80 (dd, J = 8.0, 2.2 Hz, 1H), 7.62 (d, | |
| | | | J = 8.0 Hz, 2H), 7.55 (d, J = 7.8 Hz, 2H), | |
| | | | 7.47 (d, J = 5.8 Hz, 1H), 6.68 (d, J = 11.4 | |
| | | | Hz, 1H), 6.61 (d, J = 7.6 Hz, 1H), 6.50 (d, | |
| | | | J = 5.8 Hz, 1H), 4.19-4.01 (m, 4H), 3.72 | |
| | | | (s, 3H), 3.46 (s, 3H), 3.27 (s, 1H), 2.92 (t, | |
| | | | J = 12.4 Hz, 2H), 2.17 (d, J = 11.6 Hz, | |
| | | | 2H), 1.8 (d, J = 10.2 Hz, 2H). | |
| | | 266 | ¹H NMR (400 MHz, DMSO-d₆) δ 10.93 | 608.3 |
| | | | (s, 1H), 9.14 (s, 1H), 8.96 (s, 1H), 8.69 (s, | |
| | | | 1H), 8.50 (d, J = 2.0 Hz, 1H), 8.03 (d, J = | |
| | | | 9.2 Hz, 2H), 7.96 (s, 1H), 7.84 (dd, J = | |
| | | | 8.0, 2.0 Hz, 1H), 7.63 (d, J = 7.6 Hz, 1H), | |
| | | | 7.50 (d, J = 15.2 Hz, 1H), 6.95 (d, J = 15.2 | |
| | | | Hz, 1H), 6.78 (d, J = 8.4 Hz, 1H), 6.48 − | |
| | | | 6.37 (m, 2H), 4.13 (d, J = 12.5 Hz, 4H), | |
| | | | 3.71 (s, 3H), 3.46 (s, 3H), 3.32 (s, 1H), | |
| | | | 2.91 (t, J = 12.4 Hz, 2H), 2.14 (s, 2H), | |
| | | | 1.70 (s, 2H). | |

### Example 267

(E)-3-(4-(((1-(3-Cyano-2-(6-cyanopyridin-3-yl)-3′-hydroxy-4′-methoxy-[1,l′-biphenyl]-4-yl)piperidin-4-yl)amino)methyl)phenyl)-N-hydroxyacrylamide formate was prepared according to the synthetic method of Example 84, and the structure and characterization data are as follows:

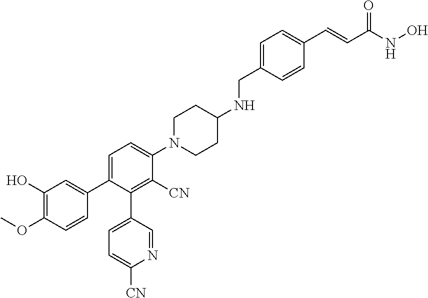 [SMILES: COc1ccc(-c2ccc(N3CCC(NCc4ccc(/C=C/C(=O)NO)cc4)CC3)c(C#N)c2-c2ccc(C#N)nc2)cc1O]

¹H NMR (400 MHZ, DMSO-d₆) δ 10.78 (s, 1H), 9.03 (m, 2H), 8.58 (d, J=2.0 Hz, 1H), 8.14 (s, 1H), 8.09 (d, J=8.0 Hz, 1H), 7.99 (dd, J=8.0, 2.1 Hz, 1H), 7.65-7.43 (m, 6H), 7.33 (d, J=8.4 Hz, 1H), 6.77 (d, J=8.4 Hz, 1H), 6.54-6.36 (m, 3H), 4.11 (s, 2H), 3.71 (s, 3H), 3.64 (d, J=11.6 Hz, 2H), 3.07 (s, 1H), 2.94 (t, J=11.6 Hz, 2H), 2.18 (d, J=12.0 Hz, 2H), 1.73 (s, 2H).

ESI-MS (m/z)=601.3 [M+H]⁺. Examples 268-271 were prepared similarly according to the synthetic method of Example 76 (the separation method for the compounds: hydrochloride and formate were prepared by separation method 1 and 3, respectively), and the structure and characterization data are as follows:

| Example | Chemical name | Structure |
|---|---|---|
| 268 | (E)-3-(4-(((1-(6-Cyano- 3′-fluoro-5′-(3-hydroxy- 4-methoxyphenyl)- [3,4′-bipyridin]-2′- yl)piperidin-4- yl)amino)methyl) phenyl)-N- hydroxyacrylamide formate | 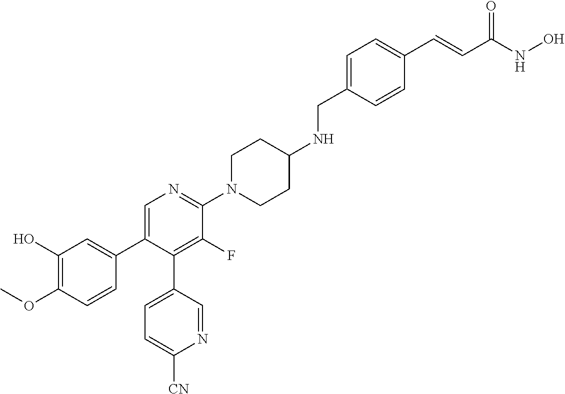 [SMILES: COc1ccc(-c2cnc(N3CCC(NCc4ccc(/C=C/C(=O)NO)cc4)CC3)c(F)c2-c2ccc(C#N)nc2)cc1O] |
| 269 | (E)-3-(4-(((1-(6-Cyano- 3′-fluoro-5′-(3-hydroxy- 4-methoxyphenyl)- [3,4′-bipyridin]-2′- yl)piperidin-4- yl)amino)methyl)-3- fluorophenyl)-N- hydroxyacrylamide hydrochloride | 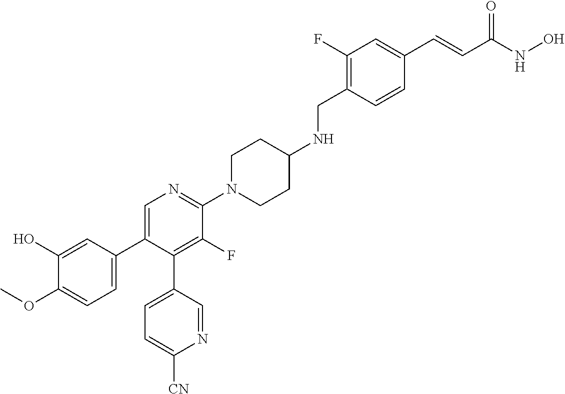 [SMILES: COc1ccc(-c2cnc(N3CCC(NCc4ccc(/C=C/C(=O)NO)cc4F)CC3)c(F)c2-c2ccc(C#N)nc2)cc1O] |
| 270 | (E)-3-(4-(((1-(6-Cyano- 3′-fluoro-5′-(2-fluoro-5- hydroxy-4- methoxyphenyl)-[3,4′- bipyridin]-2′- yl)piperidin-4- yl)amino)methyl) phenyl)-N- hydroxyacrylamide formate | 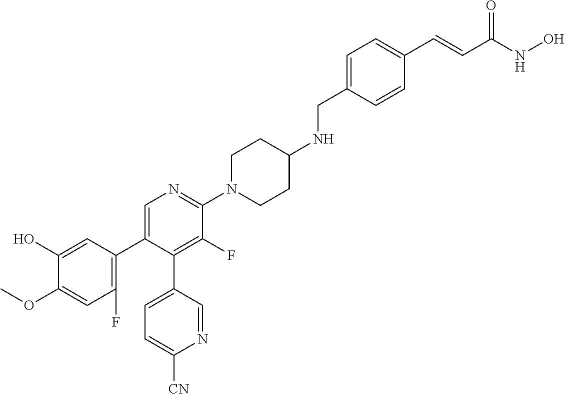 [SMILES: COc1cc(F)c(-c2cnc(N3CCC(NCc4ccc(/C=C/C(=O)NO)cc4)CC3)c(F)c2-c2ccc(C#N)nc2)cc1O] |
| 271 | (Z)-3-(4-(((1-(6-Cyano- 3′-fluoro-5′-(3-hydroxy- 4-methoxyphenyl)- [3,4′-bipyridin]-2′- yl)piperidin-4- yl)amino)methyl)phenyl- 2-fluoro-N- hydroxyacrylamide formate | 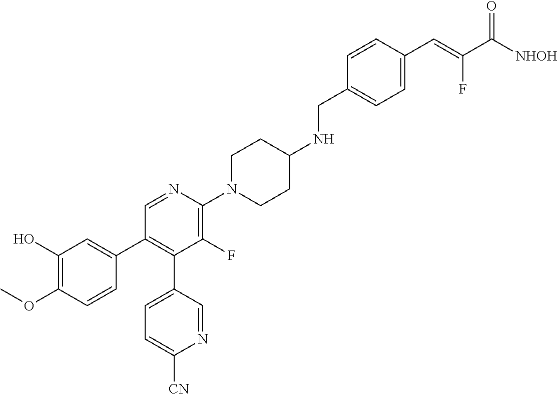 |

| | | MS |
|---|---|---|
| Example | ¹H NMR | (M + H)⁺ |
| 268 | ¹H NMR (400 MHz, DMSO-d₆) | 595.2 |
| | δ ppm: 10.78 (s, 1H), 9.02 (s, | |
| | 2H), 8.56 (s, 1H), 8.14 (d, J = 1.8 | |
| | Hz, 1H), 8.13 − 8.02 (m, 2H), | |
| | 7.95 (d, J = 8.2 Hz, 1H), 7.59 (d, | |
| | J = 7.7 Hz, 2H), 7.55 − 7.38 (m, | |
| | 3H), 6.86 − 6.76 (m, 1H), 6.54 − | |
| | 6.38 (m, 3H), 4.05 (d, J = 9.0 Hz, | |
| | 4H), 3.72 (d, J = 1.9 Hz, 3H), | |
| | 3.08 (s, 1H), 2.99 (t, J = 12.5 Hz, | |
| | 2H), 2.10 (d, J = 12.3 Hz, 2H), | |
| | 1.62 (d, J = 12.4 Hz, 2H). | |
| 269 | ¹H NMR (400 MHz, Methanol- | 613.2 |
| | d₄) δ ppm, 8.66 (s, 1H), 8.56 (s, | |
| | 1H), 8.48 (s, 1H), 8.21-8.03 (m, | |
| | 2H), 7.99-7.88 (m, 1H), 7.64 (t, | |
| | J = 7.8 Hz, 1H), 7.47 (q, J = 9.4, | |
| | 8.4 Hz, 2H), 6.81 (d, J = 8.4 Hz, | |
| | 1H), 6.54 (d, J = 15.8 Hz, 1H), | |
| | 6.44 (d, J = 6.6 Hz, 2H), 4.07 (d, | |
| | J = 16.2 Hz, 4H), 3.72 (s, 3H), | |
| | 3.20 (s, 1H), 3.00 (t, J = 12.4 Hz, | |
| | 2H), 2.12 (d, J = 12.2 Hz, 2H), | |
| | 1.65 (s, 2H). | |
| 270 | ¹H NMR (400 MHz, DMSO-d₆) | 613.2 |
| | δ ppm: 10.77 (s, 1H), 9.09 (s, | |
| | 2H), 8.58 (s, 1H), 8.14 (s, 1H), | |
| | 8.06 (t, J = 4.1 Hz, 2H), 7.92 (dd, | |
| | J = 8.0, 2.1 Hz, 1H), 7.59 (d, J = | |
| | 7.9 Hz, 2H), 7.55 − 7.39 (m, 2H), | |
| | 6.67 (dd, J = 24.9, 9.5 Hz, 2H), | |
| | 6.48 (d, J = 15.8 Hz, 1H), 4.21 − | |
| | 3.96 (m, 4H), 3.72 (s, 3H), 3.04 | |
| | (s, 1H), 3.00 (d, J = 12.3 Hz, 2H), | |
| | 2.09 (d, J = 12.2 Hz, 2H), 1.61 (d, | |
| | J = 12.2 Hz, 2H). | |
| 271 | ¹H NMR (400 MHz, Methanol- | |
| | d₄) δ ppm 8.53 (s, 1H), 8.49 (d, | |
| | J = 2.2 Hz, 1H), 8.10 (d, J = 1.8 | |
| | Hz, 1H), 7.84 (t, J = 1.4 Hz, 2H), | |
| | 7.67 (dd, J = 34.6, 8.0 Hz, 2H), | |
| | 7.50 (dd, J = 32.0, 7.8 Hz, 2H), | |
| | 6.96- 6.73 (m, 2H), 6.56-6.44 (m, | |
| | 2H), 4.30-4.17 (m, 4H), 3.82 (s, | |
| | 3H), 3.32 (s, 1H),3.05 (t, J = 12.4 | |
| | Hz, 2H), 2.23 (d, J = 12.2 Hz, | |
| | 2H), 1.77 (d, J = 10.6 Hz, 2H). | |

### Example 272

((E)-3-(4-(((1-(6-Cyano-5′-(3-hydroxy-4-methoxyphenyl)-[3,4′-bipyridin]-2′-yl)piperidin-4-yl)amino)methyl)phenyl)-N-hydroxyacrylamide formate was prepared according to the synthetic method of Example 80, and the structure and characterization data are as follows:

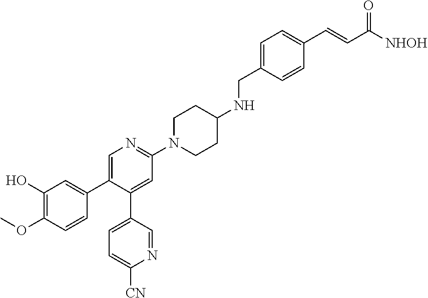

¹H NMR (400 MHZ, Methanol-d₄) δ 8.52 (d, J=1.5 Hz, 1H), 8.49 (s, 1H), 8.17 (d, J=1.8 Hz, 1H), 7.81 (d, J=1.5 Hz, 2H), 7.68 (d, J=7.7 Hz, 2H), 7.64-7.52 (m, 3H), 7.01 (d, J=4.1 Hz, 1H), 6.88-6.82 (m, 1H), 6.58-6.46 (m, 3H), 4.58 (d, J=13.5 Hz, 2H), 4.32 (s, 2H), 3.83 (s, 3H), 3.51 (d, J=17.8 Hz, 1H), 3.07 (t, J=12.7 Hz, 2H), 2.29 (d, J=12.0 Hz, 2H), 1.74 (d, J=12.4 Hz, 2H).

ESI-MS (m/z)=577.3 [M+H]⁺.

### Bioassays

### 1. In Vitro Enzyme Inhibition Assay—LSD1

The effect of the compounds on LSD1 enzyme activity was examined using the HTRF technique to assess the level of inhibition. Initially, a master solution of 90 μM test compound (dissolved in DMSO) was prepared and sequentially diluted with DMSO in a 5-fold concentration gradient to yield a total of 9 concentrations of the compound working solution 1 (90×). Subsequently these 9 concentrations of working solution 1 were sequentially diluted by a 30-fold concentration gradient, i.e., 2 μL of working solution 1 was aspirated and added to 58 μL of Buffer, mixed on a vortex mixer with sufficient shaking to yield 9 concentrations of the screening test compound working solution 2 (3×). In a 384-well shallow white plate, 2 μL of the compound working solution 2 (3×) was added to each well, followed by the addition of 2 μL of a 6×LSD1 (Activemotif, 31426) and 6×FAD (Sigma, F8384) pre-mixed (1:1) solution. The mixture was incubated at room temperature for 15 minutes. Then 2 μL of the 3×H3K4mel (Anaspec, AS-64355-025) substrate solution was added to each well, mixed evenly, and incubated for 60 minutes. Afterward, 2 μL of a termination solution (containing 5.4 mM 2-PCPA) was added to each well, mixed evenly, and incubated for another 15 minutes. Finally 4 μL of a pre-mixed antibody solution of Eu-anti H3K4 (PerkinElmer, TRF0404-D) and allophycocyanin (Prozyme, PJ27S) (1:1) was added to each well, mixed evenly, and incubated for 60 minutes. The 384-well plate was then read on a multifunctional microplate reader, with the excitation wavelength set to 337 nm. Readings were recorded at 620 nm and 665 nm. The data results were presented as the ratio of the 665 nm signal value to the 620 nm signal value in each well, calculated using the formula: Ratio=10⁴×665 nm signal value/620 nm signal value. The inhibition rate was calculated through the following formula:

$$\% Inhibition \text{} rate \text{} = \text{} [ ( {Ratio}_{negative} - {Ratio}_{compound} ) \text{} / ( {Ratio}_{negative} - {Ratio}_{B l a n k} ) ] \times 100$$

Note: ‘Negative’ refers to the group without the inhibitor; ‘Blank’ refers to the group without the enzyme.

The IC₅₀ value was calculated by fitting the inhibition rate by GraphPad Prism software, employing log (inhibitor) vs. response-Variable Slope (four parameters) model.

### 2. In Vitro Enzyme Inhibition Assay—HDAC

The effect of the compound on the enzymatic activity of pan-HDAC and two isoforms (HDAC1 and HDAC8) was examined using the HDAC-Glo I/II Assay and Screening System assay to assess the level of inhibition of HDAC protease activity and selectivity in each isoform. The proteins and detection reagents were from the HDAC-Glo™ I/II Assay and Screening System (Promega). A 1 mM stock solution of the compound was prepared in DMSO and diluted in a 5-fold gradient to create a series of 8 concentrations, and a specific volume of the highest concentration was diluted 25 times with HDAC Buffer to obtain 4× working solution of the compound. In a 384-well plate, 5 μL of the 4×HDAC enzyme solution and 5 μL of the test compound (4×) were sequentially added to each well, mixed evenly, and incubated at room temperature for 30 minutes. Subsequently 10 μL of 2× Developer reagent was added to each well, mixed evenly, and incubated at room temperature for 15-45 minutes. The 384-well plate was then read on a multifunctional microplate reader, with the Luminescence channel selected to record Luminescence values (RLU). The inhibition rate was calculated using the following formula:

$$\% Inhibition rate = ⌊ \frac{{RLU}_{Negative control} - {RLU}_{Test compoud}}{{RLU}_{Negative control} - {RLU}_{B l a n k}} ⌋ ⋆ 100$$

Note: ‘Negative’ refers to the group without the inhibitor; ‘Blank’ refers to the group without the enzyme.

The IC₅₀ value was calculated by fitting the inhibition rate by GraphPad Prism software, employing log (inhibitor) vs. response-Variable Slope (four parameters) model.

TABLE I
Enzymatic Activity of the Compounds

| | LSD1 | HDAC IC₅₀ (nM) | | |
|---|---|---|---|---|
| Example | IC₅₀(nM) | HDAC 1 | HDAC 8 | Pan-HDAC |
| 1 | 1.0 | 68.1 | 214.8 | 17.5 |
| 2 | 1.58 | 92.3 | 289 | 66.9 |
| 3 | 13.7 | N.T | N.T | 29 |
| 4 | 11.9 | N.T | N.T | 12 |
| 5 | 35.2 | N.T | N.T | 15 |
| 6 | 254 | N.T | N.T | 134 |
| 7 | 36 | N.T | N.T | 41.3 |
| 8 | 18.6 | N.T | N.T | 21.7 |
| 9 | 1.82 | N.T | N.T | 25.3 |
| 10 | 75.4 | N.T | N.T | 39 |
| 11 | 74.5 | N.T | N.T | 116.5 |
| 12 | 0.92 | N.T | N.T | 7.31 |
| 13 | 1.95 | N.T | N.T | 3.32 |
| 15 | 1.45 | N.T | N.T | 27.5 |
| 16 | 33.2 | N.T | N.T | 4.23 |
| 17 | 23.8 | N.T | N.T | 21.6 |
| 19 | 0.86 | N.T | 221.1 | 173.9 |
| 20 | 16.9 | 311.3 | 445.5 | 96.5 |
| 21 | 0.05 | 400 | 275.1 | 119 |
| 22 | 0.31 | N.T | N.T | 144.4 |
| 23 | 0.23 | 198.1 | 320.2 | 74.4 |
| 24 | 0.41 | 8.28 | 117.7 | 5.83 |
| 25 | 0.23 | N.T | 128.9 | 6.66 |
| 26 | 0.12 | N.T | N.T | 35 |
| 27 | 1.38 | N.T | 185.7 | 134.2 |
| 28 | 0.64 | 607.8 | 2331 | 269.7 |
| 29 | 0.96 | 123.2 | 696.2 | 66.9 |
| 30 | 4.25 | 39.9 | 65.8 | 18.1 |
| 31 | 311 | 1.78 | 263.4 | 0.25 |
| 32 | 0.35 | N.T | 279.4 | 14 |
| 33 | 3.71 | 56.4 | 182.4 | 34.2 |
| 34 | 1.13 | 1063 | 353 | 551.8 |
| 35 | 4.63 | N.T | N.T | 266.1 |
| 36 | 1.18 | 118.2 | 97.7 | 75.1 |
| 37 | 1.95 | 4.7 | 93.6 | 21.2 |
| 39 | 0.15 | 335 | 207.3 | 166.6 |
| 40 | 8.25 | 108.3 | 391.6 | 54.4 |
| 41 | 0.13 | 1124 | 1368 | 1014 |
| 42 | 0.33 | 7.33 | 327.5 | 9.25 |
| 43 | 0.62 | 5.54 | 306.2 | 4.3 |
| 44 | 2.58 | 45.8 | 187.6 | 27.9 |
| 45 | 0.36 | 70.6 | 156.4 | 32.1 |
| 50 | 4.33 | 117.5 | 169.5 | 70.7 |
| 52 | 47.8 | 15.9 | 140.2 | 4.22 |
| 54 | 0.62 | 58.2 | 125.4 | 34.4 |
| 56 | 91.5 | 641.8 | 892.6 | 467 |
| 57 | 0.26 | N.T | N.T | 72 |
| 58 | 0.1 | 47.4 | 135.9 | 31.9 |
| 62 | 0.49 | 17.3 | 3188 | 5.53 |
| 63 | 8.7 | 9.28 | 561.2 | 6.22 |
| 64 | 1.4 | 9.08 | 440.9 | 5.95 |
| 67 | 1.89 | N.T | N.T | 39 |
| 69 | >1000 | N.T | 198.4 | 30.1 |
| 70 | 1.18 | N.T | 26.5 | 76.6 |
| 71 | 4.61 | N.T | 124.6 | 51.8 |
| 72 | 8.42 | N.T | 159.5 | 110.5 |
| 73 | 21.2 | N.T | 151.8 | 185.7 |
| 74 | 2.26 | N.T | 207.1 | 23.1 |
| 75 | 129.2 | N.T | 211.8 | 76.6 |
| 76 | 10.7 | N.T | 157.4 | 1.59 |
| 80 | 0.78 | N.T | 164.6 | 14.9 |
| 81 | 140.1 | N.T | 695.3 | 1221 |
| 83 | 8.51 | N.T | 374.1 | 438 |
| 84 | 0.62 | N.T | 73.6 | 5.45 |
| 85 | 208.9 | N.T | 541.9 | 110.2 |
| 86 | 9.46 | N.T | 578 | 13.8 |
| 88 | 28.7 | 0.21 | 178.2 | 5.09 |
| 90 | 8.01 | 10.6 | 63.5 | 19.5 |
| 92 | 1.41 | N.T | 622.7 | 340.2 |
| 94 | 172.8 | N.T | 256.5 | 110.1 |
| 95 | 63.6 | N.T | 87.2 | 49 |
| 96 | 39.9 | N.T | 16.3 | 16.4 |
| 97 | 1.06 | 8.3 | 92.4 | 13.9 |
| 98 | 160 | N.T | 480.3 | 394.5 |
| 99 | 9.5 | N.T | 120.4 | 50.9 |
| 100 | 49.5 | N.T | 36 | 35.5 |
| 101 | 0.52 | 13.6 | 206.7 | 27.5 |
| 102 | 1.31 | 16.3 | 178.5 | 45.2 |
| 103 | 14.2 | N.T | 158.8 | 62.3 |
| 104 | 0.71 | 5.3 | 70.1 | 10.7 |
| 105 | 1.24 | 120 | 185 | 114.1 |
| 106 | 2.62 | 3.81 | 13.1 | 4.85 |
| 107 | 1.29 | N.T | 50.6 | 378.2 |
| 108 | 2.3 | 25.8 | 93.1 | 26.6 |
| 109 | 4.19 | N.T | 391.4 | 657.1 |
| 110 | 1.05 | 156.4 | 137.3 | 57.3 |
| 111 | 1.56 | 83.1 | 357.6 | 65.3 |
| 112 | 2.66 | 26.3 | 81.4 | 26.9 |
| 113 | 2 | 17.3 | 72.7 | 25 |
| 114- | 0.97 | 177.7 | 571.3 | 175.3 |
| 115 | 0.16 | N.T | 37.1 | 20.6 |
| 116 | 1.82 | 5.59 | 212.3 | 12.7 |
| 117 | 0.36 | 0.59 | 13 | 0.8 |
| 118 | 0.58 | 0.44 | 30.3 | 0.75 |
| 119 | 1.98 | N.T | 245.8 | 33.6 |
| 120 | 0.33 | N.T | 1949 | 1314 |
| 121 | 0.35 | N.T | 17.2 | 404.1 |
| 123 | 0.74 | N.T | 39 | 7.34 |
| 124 | 1.63 | 3.95 | 178.7 | 7.69 |
| 125 | 0.74 | N.T | 23.4 | 226 |
| 126 | 1.11 | N.T | 132.3 | 197.3 |
| 127 | 1.2 | 5.82 | 422.8 | 12.4 |
| 128 | 0.39 | N.T | 222.7 | 5 |
| 129 | 1.47 | N.T | 321.9 | 18.2 |
| 130 | 1.89 | 0.77 | 219.9 | 6.13 |
| 131 | 1.31 | 0.22 | 46.6 | 1.57 |
| 132 | 10.8 | N.T | 171.3 | 3.86 |
| 133 | 22 | 1.45 | 139.5 | 11.78 |
| 134 | 13.6 | 0.32 | 46 | 3.55 |
| 135 | 3.16 | 17.26 | 1009 | 39.7 |
| 136 | 17.2 | N.T | 77.7 | 10.4 |
| 137 | 27.8 | 0.52 | 106.8 | 6.04 |
| 138 | 21.4 | N.T | 401.4 | 61 |
| 139 | 43.4 | 0.53 | 147 | 6.82 |
| 140 | 44.2 | N.T | 465.5 | 5.64 |
| 141 | 1.72 | N.T | 53 | 5.62 |
| 142 | 1.8 | 0.6 | 129 | 1.27 |
| 143 | 0.1 | 1.25 | 111.1 | 1.7 |
| 144 | 1.07 | 0.21 | 91.6 | 0.86 |
| 145 | 3.35 | N.T | 74.9 | 5.23 |
| 147 | 153.4 | N.T | 1174 | 33.2 |
| 148 | 1.05 | 0.43 | 58.4 | 2.47 |
| 149 | 0.54 | 0.5 | 58.9 | 1.05 |
| 150 | 0.59 | 0.21 | 89.3 | 1.3 |
| 151 | 2.7 | N.T | 618.1 | 27.4 |
| 152 | 1.15 | N.T | 131.4 | 2.57 |
| 153 | 0.74 | N.T | 400.4 | 5.49 |
| 154 | 0.44 | N.T | 309.2 | 4.3 |
| 155 | 0.58 | N.T | 23.7 | 20 |
| 156 | 2.53 | N.T | 730.8 | 8.88 |
| 158 | 0.73 | N.T | 87.8 | 0.61 |
| 159 | 1.82 | N.T | 83.5 | 2.71 |
| 160 | 1.12 | N.T | 183.5 | 1.48 |
| 161 | 0.58 | N.T | 303.8 | 6.83 |
| 162 | 0.4 | N.T | 209.2 | 3.34 |
| 164 | 0.44 | N.T | 332.5 | 38.1 |
| 165 | 1.35 | N.T | 144.8 | 7.31 |
| 166 | 15.1 | N.T | 64 | 2.27 |
| 167 | 0.37 | N.T | 823.5 | 21.9 |
| 168 | 1.34 | N.T | 249.8 | 7.35 |
| 169 | 0.42 | N.T | 1400 | 51.7 |
| 170 | 0.43 | N.T | 62.7 | 2 |
| 171 | 0.55 | N.T | 53.1 | 1.68 |
| 172 | 0.74 | N.T | 327.4 | 162.4 |
| 173 | 0.82 | N.T | 251.9 | 114 |
| 176 | 0.69 | N.T | 507.8 | 38.2 |
| 177 | 963 | N.T | 580.5 | 40.2 |
| 178 | 0.65 | N.T | 1713 | 588.3 |
| 179 | 100.3 | 1.74 | 374.5 | 3.1 |
| 180 | 0.11 | N.T | 1235 | 23.4 |
| 181 | 0.13 | 0.08 | 75.1 | 2.11 |
| 182 | 0.09 | 1.3 | 30.1 | 3.02 |
| 183 | 0.09 | N.T | 3723 | 969.9 |
| 184 | 0.09 | 1.69 | 45.2 | 6.58 |
| 185 | 3.9 | N.T | 2163 | 60.1 |
| 186 | 10.2 | N.T | 50.9 | 11.9 |
| 187 | 4.11 | 1.98 | 15.5 | 3.71 |
| 188 | 0.3 | 3.06 | 198.4 | 5.07 |
| 189 | 4.11 | 13.4 | 129.9 | 7.8 |
| 190 | 2.85 | N.T | 336 | 5.96 |
| 191 | 2.24 | 0.54 | 37.5 | 2.17 |
| 192 | 0.45 | 1.49 | 64.9 | 2.75 |
| 193 | 1.71 | N.T | 296.9 | 8.36 |
| 194 | 1.09 | N.T | 542.7 | 7.27 |
| 195 | 4.12 | N.T | 172.1 | 11.8 |
| 196 | 6.68 | N.T | 26.5 | 5.69 |
| 197 | 0.49 | N.T | 30.1 | 7.48 |
| 198 | 0.40 | 4.85 | 67.2 | 6.35 |
| 199 | 0.54 | 1.92 | 33.9 | 2.77 |
| 200 | 0.45 | 2.95 | 126.7 | 4.18 |
| 201 | 0.22 | 4.81 | 438.5 | 7.71 |
| 202 | 74.2 | 0.88 | 9.03 | 1.85 |
| 203 | 1.03 | 1.58 | 25.3 | 1.39 |
| 204 | 1.58 | N.T | 26.2 | 2.37 |
| 205 | 1.91 | 3.08 | 100.5 | 4.37 |
| 206 | 2.03 | 32.9 | 32.5 | 11.5 |
| 207 | 6.65 | 2.51 | 170.4 | 12.4 |
| 208 | 0.73 | 1.74 | 17.2 | 1.13 |
| 209 | 0.6 | N.T | 3.09 | 163 |
| 210 | 1.13 | 4.44 | 456 | 2.5 |
| 211 | 0.31 | N.T | 83.5 | 2.04 |
| 212 | 0.04 | N.T | 41.5 | 6.55 |
| 213 | 2.12 | N.T | N.T | 19.7 |
| 214 | 4.98 | N.T | N.T | 142.9 |
| 215 | 0.39 | N.T | N.T | 68 |
| 216 | 0.15 | N.T | N.T | 3.57 |
| 217 | 13.6 | 0.73 | N.T | 0.25 |
| 218 | 10.2 | 1.77 | N.T | 1.01 |
| 219 | 0.21 | N.T | N.T | 9.28 |
| 220 | 0.42 | N.T | N.T | 937 |
| 221 | 17.1 | N.T | N.T | 2.2 |
| 222 | 0.03 | N.T | N.T | 11.5 |
| 223 | 1.01 | N.T | N.T | 4.37 |
| 224 | 0.19 | N.T | N.T | 10.6 |
| 225 | 0.35 | N.T | N.T | 3.43 |
| 226 | 0.02 | N.T | N.T | 24.8 |
| 227 | 0.64 | 9.71 | N.T | 11.3 |
| 228 | 0.90 | N.T | N.T | 24.3 |
| 229 | 0.88 | N.T | N.T | 30.8 |
| 230 | 1.45 | N.T | N.T | 10.3 |
| 231 | 0.07 | N.T | 140 | 82.4 |
| 232 | 45.01 | N.T | N.T | 114 |
| 233 | 1.76 | 0.43 | 42.1 | 2.23 |
| 234 | 1.08 | N.T | 51.1 | 13.3 |
| 235 | 2.38 | N.T | N.T | 8.01 |
| 236 | 0.59 | N.T | N.T | 37 |
| 237 | 1.75 | 1.29 | N.T | 3.37 |
| 238 | 3.48 | 1.62 | N.T | 4.05 |
| 239 | 2.36 | N.T | N.T | 10.2 |
| 240 | 0.82 | 1.85 | N.T | 4.73 |
| 241 | 2.95 | 1.86 | N.T | 7.81 |
| 242 | 2.44 | N.T | N.T | 16 |
| 243 | 13.6 | 0.6 | N.T | 1.74 |
| 244 | 0.98 | 0.51 | N.T | 1.54 |
| 245 | 24.7 | 0.64 | N.T | 3.2 |
| 246 | 0.93 | N.T | N.T | 5.38 |
| 247 | 3.49 | 0.84 | N.T | 0.87 |
| 248 | 0.81 | 0.61 | N.T | 0.62 |
| 249 | 1.1 | N.T | N.T | 0.86 |
| 250 | 17.0 | N.T | N.T | 1.08 |
| 251 | 245 | N.T | N.T | 0.59 |
| 252 | 3.24 | N.T | N.T | 1.03 |
| 253 | 342 | N.T | N.T | 0.3 |
| 254 | 35.4 | N.T | N.T | 0.41 |
| 255 | 1.54 | N.T | N.T | 0.68 |
| 256 | 28.8 | N.T | N.T | 0.26 |
| 257 | 7.93 | N.T | N.T | 0.59 |
| 258 | 1.41 | N.T | N.T | 0.30 |
| 259 | 81.2 | N.T | N.T | 0.25 |
| 260 | 2.37 | N.T | N.T | 0.77 |
| 261 | 3.18 | N.T | N.T | 1.42 |
| 262 | 7.04 | N.T | N.T | 0.61 |
| 263 | 16.8 | N.T | N.T | 0.34 |
| 264 | 25.4 | N.T | N.T | 0.89 |
| 265 | 10.2 | N.T | N.T | 0.45 |
| 266 | 24.2 | N.T | N.T | 0.16 |
| 267 | 7.36 | N.T | N.T | 1.93 |
| 268 | 147 | N.T | N.T | 0.27 |
| 269 | 193 | N.T | N.T | 0.93 |
| 270 | 24.8 | N.T | N.T | 0.32 |
| 271 | 35.1 | N.T | N.T | 0.83 |
| 272 | 25.2 | N.T | N.T | 0.30 |
| CC-90011 | 1.1 | >10000 | >10000 | >10000 |
| SAHA | >1000 | 51.8 | N.T | 38.6 |
| (Vorinostat) | | | | |

Note:
N.T means untested.

The data presented in Table I shows that the compounds of the present disclosure has a potent inhibitory effect on both LSD1 and HDAC enzymes.

The structure of SAHA is:

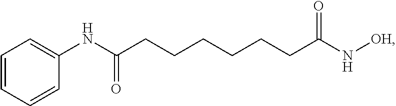 [SMILES: O=C(CCCCCCC(=O)Nc1ccccc1)NO]

and it is brought from Selleck company;

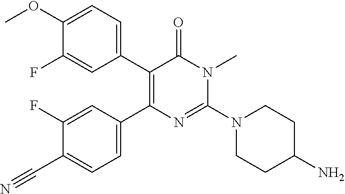 [SMILES: COc1ccc(-c2c(-c3ccc(C#N)c(F)c3)nc(N3CCC(N)CC3)n(C)c2=O)cc1F]

The structure of CC-90011 is:

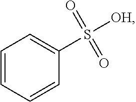

and it is synthesized according to the literature methods (Kanouni, T., et al, J. Med. Chem., 2020, 63, 14522-14529).

### 3. In Vitro Cell Inhibition Assay—H1417 Cell Proliferation

The inhibitory effect of the compounds on cell proliferation was assessed by detecting the number of viable cells using the CellTiter-Glo® reagent. NCI-H1417 cells in the logarithmic growth phase were collected and inoculated into transparent-bottom 96-well plates at a density of 7×10³ cells/well with 100 μL of medium, and incubated at 37° C., 5% CO₂ overnight. The compounds were sequentially diluted in a 5-fold concentration gradient with DMSO to obtain 8 different concentrations o. These were diluted with BEGM (containing 10% FBS) cell culture medium to obtain the compound working solution (2×), which was added to the cell supernatant at 100 μL per well. The incubation continued for 7 days at 37° C., 5% CO₂. The plate was then removed and equilibrated to room temperature (25° C.) along with the CellTiter-Glo® mixing reagent for 10-30 minutes. After centrifugation, 100 μL of the culture medium was carefully aspirated from each well, and 85 μL of CellTiter-Glo® reagent was added. The cells were thoroughly mixed with the CellTiter-Glo® mixing reagent using a microplate shaker for 2 min. and then incubated at room temperature for 10 min. The 96-well plate was subsequently placed on a multifunctional microplate reader to record the Luminescence values (RLU).
The inhibition rate was calculated using the following formula:

$$\% Inhibition rate = ⌊ \frac{{RLU}_{Negative control} - {RLU}_{Test compoud}}{{RLU}_{Negative control} - {RLU}_{B l a n k}} ⌋ ⋆ 100$$

Note: ‘Negative’ refers to the group without inhibitors; ‘Blank’ refers to the group without cells.

The IC₅₀ value was determined by fitting the inhibition rate by GraphPad Prism software.

### 4. In Vitro Cell Inhibition Assay—NCI-H69 Cell Proliferation

The inhibitory effect of the compounds on cell proliferation was assessed by detecting the number of viable cells using the CellTiter-Glo® reagent. NCI-H69 cells in the logarithmic growth phase were collected and inoculated into transparent bottom 96-well plates at a density 1×10³ cells per well with 100 μL medium, and incubated at 37° C., 5% CO₂ overnight. The compounds were sequentially diluted in a 5-fold concentration gradient with DMSO to obtain 8 different concentrations These were then diluted with RPMI-640 (containing 20% FBS) cell culture medium to obtain the compounds working solution (2×), which was added to the cell supernatant at 100 μL per well. The incubation continued for 7 days at 37° C., 5% CO₂. The plate was then removed and equilibrated to room temperature (25° C.) along with the CellTiter-Glo® mixing reagent for 10-30 minutes. After centrifugation at 1200 rpm for 5 minutes, 120 μL of the culture medium was carefully aspirated from each well, and 60 μL of CellTiter-Glo® reagent was added. The cells were thoroughly mixed with the CellTiter-Glo® mixing reagent using a microplate shaker for 2 min. and then incubated at room temperature for 10 min. The 96-well plate was subsequently placed on a multifunctional microplate reader to record the Luminescence values (RLU).

The inhibition rate was calculated using the following formula:

$$\% Inhibition rate = ⌊ \frac{{RLU}_{Negative control} - {RLU}_{Test compoud}}{{RLU}_{Negative control} - {RLU}_{B l a n k}} ⌋ ⋆ 100$$

Note: ‘Negative’ refers to the group without inhibitors; ‘Blank’ refers to the group without cells.

The IC₅₀ value was determined by fitting the inhibition rate by GraphPad Prism software.

### 5. In Vitro Cell Inhibition Assay—DU145 Cell Proliferation

The inhibitory effect of the compounds on cell proliferation was assessed by detecting the number of viable cells using the CellTiter-Glo® reagent. DU145 cells in the logarithmic growth phase were collected and inoculated into transparent bottom 96-well plates at a density 1×10³ cells per well with 100 μL medium, and incubated at 37° C., 5% CO₂ overnight. The compounds were sequentially diluted in a 5-fold concentration gradient with DMSO to obtain 8 different concentrations. These were then diluted with RPMI-640 (containing 10% FBS) cell culture medium to obtain the compound working solution (2×), which was added to the cell supernatant at 100 μL per well. The incubation continued for 6 days at 37° C., 5% CO₂. The plate was then removed and equilibrated at room temperature (25° C.) along with the CellTiter-Glo® mixing reagent for 10-30 minutes. After centrifugation, 120 μL of the culture medium was carefully aspirated from each well, and 60 μL of CellTiter-Glo® reagent was added. The cells were thoroughly mixed with the CellTiter-Glo® mixing reagent using a microplate shaker for 2 min. and then incubated at room temperature for 10 min. The 96-well plate was subsequently placed on a multifunctional microplate reader to record the Luminescence values (RLU).

The inhibition rate was calculated using the following formula:

$$\% Inhibition rate = ⌊ \frac{{RLU}_{Negative control} - {RLU}_{Test compoud}}{{RLU}_{Negative control} - {RLU}_{B l a n k}} ⌋ ⋆ 100$$

Note: ‘Negative’ refers to the group without inhibitors; ‘Blank’ refers to the group without cells.

The IC₅₀ value was determined by fitting the inhibition rate by GraphPad Prism software.

### 6. In Vitro Cell Inhibition Assay—NCI-H526 Cell Proliferation

The inhibitory effect of the compounds on cell proliferation was assessed by detecting the number of viable cells using CellTiter-Glo® reagent. NCI-H526 cells in the logarithmic growth phase were collected and inoculated into transparent bottom 96-well plates at a density 1×10³ cells per well with 100 μL of medium, and incubated at 37° C., 5% CO₂ overnight. The compounds were sequentially diluted in a 5-fold concentration gradient with DMSO to obtain 8 different concentrations. These were then diluted with RPMI-640 (containing 10% FBS) cell culture medium to obtain the compound working solution (2×), which was added to the cell supernatant at 100 μL per well. The incubation continued for 5 days at 37° C., 5% CO₂. The plate was then removed and equilibrated at room temperature (25° C.) along with the CellTiter-Glo® mixing reagent for 10-30 minutes; 80 μL of the culture medium was carefully aspirated from each well, and 80 μL of CellTiter-Glo® reagent was added. The cells were thoroughly mixed with the CellTiter-Glo® mixing reagent using a microplate shaker for 2 min. and then incubated at room temperature for 10 min. The 96-well plate was subsequently placed on a multifunctional microplate reader to record the Luminescence values (RLU).

The inhibition rate was calculated using the following formula:

$$\% Inhibition rate = ⌊ \frac{{RLU}_{Negative control} - {RLU}_{Test compoud}}{{RLU}_{Negative control} - {RLU}_{B l a n k}} ⌋ ⋆ 100$$

Note: ‘Negative’ refers to the group without inhibitors; ‘Blank’ refers to the group without cells.

The IC₅₀ value was determined by fitting the inhibition rate by GraphPad Prism software.

TABLE II
Cellular Activity of the Compounds

| | H1417 | H69 | Du145 | H526 |
|---|---|---|---|---|
| Example | IC₅₀(nM) | IC₅₀(nM) | IC₅₀(nM) | IC₅₀(nM) |
| 1 | 216 | N.T | N.T | N.T |
| 2 | 51.9 | 7893 | N.T | N.T |
| 3 | 225.9 | N.T | N.T | N.T |
| 4 | 209.2 | N.T | N.T | N.T |
| 5 | 127.9 | N.T | N.T | N.T |
| 7 | 762.4 | 2544 | 2957 | N.T |
| 8 | 500.7 | 2532 | 2851 | N.T |
| 9 | 914.8 | 3095 | N.T | N.T |
| 11 | >1000 | 2355 | 2000 | N.T |
| 13 | 331.6 | 774.6 | 2768 | N.T |
| 14 | >1000 | 2525 | 3206 | N.T |
| 15 | 221.3 | 2991 | N.T | N.T |
| 16 | >1000 | 3644 | 3702 | N.T |
| 17 | >1000 | 3273 | N.T | N.T |
| 19 | 2816 | N.T | N.T | N.T |
| 20 | 845 | N.T | N.T | N.T |
| 21 | 65 | N.T | N.T | N.T |
| 22 | 106.1 | 4734 | N.T | N.T |
| 23 | 1.55 | 760.4 | N.T | N.T |
| 24 | 42.3 | N.T | N.T | N.T |
| 25 | 50.1 | N.T | N.T | N.T |
| 26 | 152 | N.T | N.T | N.T |
| 27 | 48.9 | N.T | N.T | N.T |
| 28 | 25.5 | N.T | N.T | N.T |
| 29 | 92.2 | 3205 | N.T | N.T |
| 30 | 205.8 | 1502 | 2827 | N.T |
| 31 | 51 | 410.4 | 93.6 | 37.4 |
| 32 | 54.7 | N.T | N.T | N.T |
| 33 | 140.2 | 185.6 | 1981 | 470.1 |
| 34 | 140 | N.T | N.T | N.T |
| 35 | 187.7 | N.T | N.T | N.T |
| 36 | 6.58 | 108.6 | 1013 | 664.9 |
| 37 | 6.56 | 8.52 | 26 | 10.3 |
| 39 | 5.2 | 2393 | N.T | N.T |
| 40 | 65 | 193.9 | 1642 | N.T |
| 41 | 1.29 | 1678 | 5055 | N.T |
| 42 | 0.38 | 39.9 | 107.8 | 22.8 |
| 43 | 4.96 | 38.5 | 313.7 | 35.3 |
| 44 | 16.1 | 150.1 | 566.8 | 139.3 |
| 45 | 6.48 | 150.7 | 1047 | 287.5 |
| 49 | 80.1 | 232.2 | N.T | 332.7 |
| 50 | 5.58 | 162.7 | 368.6 | 216 |
| 52 | 81.6 | 71.03 | 216.1 | 111.3 |
| 54 | 0.8 | 248.2 | 1049 | N.T |
| 56 | 5.4 | 1282 | N.T | N.T |
| 57 | 14.5 | N.T | N.T | N.T |
| 58 | 46.3 | 3000 | N.T | N.T |
| 62 | 9.86 | 3225 | N.T | 1207 |
| 63 | 72.6 | 421.7 | 1805 | N.T |
| 64 | 28.6 | 2912 | 9361 | N.T |
| 67 | 21.2 | 444.4 | N.T | N.T |
| 70 | 1.44 | 213.6 | 262 | N.T |
| 71 | 42.3 | 99.0 | 431.3 | N.T |
| 72 | 165.5 | 969.6 | 2752 | N.T |
| 73 | 125.2 | 678.0 | 2233 | N.T |
| 74 | 40.1 | 204.8 | 552.7 | N.T |
| 75 | 473.0 | 402.8 | 677.4 | N.T |
| 76 | 11.7 | 3.96 | 11 | 8.27 |
| 77 | 660.9 | 120.9 | 319.5 | N.T |
| 80 | 14.0 | 27.8 | 56.5 | 29.3 |
| 81 | 73.9 | 516.2 | 776 | N.T |
| 82 | 11.5 | 267.6 | 352.3 | N.T |
| 83 | 76.8 | 1140 | 2820 | N.T |
| 84 | 4.0 | 32.9 | 115.5 | 68.4 |
| 85 | 256.3 | 407.1 | 1547 | N.T |
| 86 | 15.4 | 144.8 | N.T | N.T |
| 88 | 9.28 | 5.25 | 14.2 | 10.7 |
| 89 | >1000 | 120.5 | 390.1 | N.T |
| 90 | 42.0 | 6.49 | 45.3 | 34.6 |
| 91 | 623.4 | 905.6 | 2627 | N.T |
| 92 | 63.0 | 496.9 | 2373 | N.T |
| 93 | 343.1 | 1282 | 2841 | N.T |
| 94 | 160.5 | 190.1 | 495.2 | N.T |
| 95 | 334.6 | 304.3 | 628 | N.T |
| 96 | 74.2 | 101.0 | 148.6 | 107.6 |
| 97 | 8.93 | 8.76 | 20.96 | 14.2 |
| 98 | 706.6 | 527.9 | 2227 | N.T |
| 99 | 11.8 | 97.3 | 122.4 | N.T |
| 100 | 117.9 | 103.7 | 312.7 | N.T |
| 101 | 4.31 | 6.02 | 20.6 | 8.26 |
| 102 | 3.10 | 0.36 | 7.43 | 4.27 |
| 103 | 43.3 | 83.6 | 254.2 | N.T |
| 104 | 6.58 | 2.29 | 24.8 | 16.9 |
| 105 | 4.48 | 102.1 | 317.1 | 124.3 |
| 106 | 3.29 | 3.48 | 13.6 | 7.09 |
| 107 | 10.4 | 1041 | 2326 | N.T |
| 108 | 4.4 | 16 | 28.6 | 13.8 |
| 109 | 2.76 | 411.3 | 3816 | N.T |
| 110 | 2.7 | 82.7 | 186.6 | 127.2 |
| 111 | 1.98 | 3.41 | 14.2 | 16.2 |
| 112 | 4.14 | 32.7 | 86.8 | 63.5 |
| 113 | 29.8 | 76.3 | 130 | 80 |
| 114 | 9.40 | 78.2 | 390.7 | 154.6 |
| 115 | 2.25 | 3340 | 6337 | N.T |
| 116 | 4.89 | 33.8 | 82 | 33.8 |
| 117 | 1.70 | 4.21 | 10.4 | 4.54 |
| 118 | 2.80 | 3.24 | 11.8 | 8.09 |
| 119 | 1.68 | 121.7 | 487.4 | 194 |
| 120 | 11.1 | 938.1 | 3767 | N.T |
| 121 | 15.6 | 127.3 | 2224 | N.T |
| 123 | 4.2 | 76.3 | 111.7 | 71.3 |
| 124 | 3.34 | 18.3 | 30.5 | 20.6 |
| 125 | 12.0 | 1949 | 3035 | N.T |
| 126 | 1.76 | 283.5 | 715 | 280 |
| 127 | 2.65 | 8.85 | 15.5 | 15.4 |
| 128 | 2.01 | 130.3 | 238.8 | N.T |
| 129 | 4.38 | 25.8 | 86.9 | N.T |
| 130 | 1.09 | 25.2 | 25 | 22.8 |
| 131 | 0.62 | 2.59 | 7.09 | 3.54 |
| 132 | 13.2 | 102.9 | 234.9 | N.T |
| 133 | 5.42 | 25.5 | 128.3 | 45.8 |
| 134 | 5.10 | 8.37 | 20.3 | 13.8 |
| 135 | 6.00 | 199 | 1585 | 200.3 |
| 136 | 29.1 | 93.7 | 364.4 | N.T |
| 137 | 5.43 | 7.01 | 63.5 | 22.9 |
| 138 | 30.7 | 91.6 | 371.6 | N.T |
| 139 | 10.1 | 13.2 | 12.6 | 17.1 |
| 140 | 219.9 | 405.1 | 5186 | N.T |
| 141 | 31.0 | 95.8 | 129.4 | N.T |
| 142 | 7.59 | 4.73 | 4.24 | 4.61 |
| 143 | 1.03 | 16.8 | 23.7 | 18.1 |
| 144 | 11.2 | 11.2 | 9.28 | 8.4 |
| 145 | 18.6 | 39.3 | 37.8 | N.T |
| 147 | 255.2 | 440.2 | 657.9 | N.T |
| 148 | 2.64 | 13.1 | 21.8 | 6.22 |
| 149 | 2.08 | 23.6 | 33.9 | 14.5 |
| 150 | 5.53 | 16.9 | 9.26 | 9.84 |
| 151 | 10.4 | 189 | 307.7 | N.T |
| 152 | 4.91 | 208 | 223 | N.T |
| 153 | 6.21 | 65.5 | N.T | N.T |
| 154 | 1.41 | 16.5 | N.T | 21.7 |
| 155 | 11.5 | 314.3 | N.T | N.T |
| 156 | 6.64 | 58.1 | N.T | 80.4 |
| 158 | 93.6 | 250.5 | N.T | N.T |
| 159 | 3.50 | 17.9 | N.T | 16.1 |
| 160 | 5.59 | 80.6 | N.T | N.T |
| 161 | 3.13 | 65 | N.T | N.T |
| 162 | 2.98 | 21.7 | N.T | 18.5 |
| 163 | 4.97 | 1398 | N.T | N.T |
| 164 | 2.66 | 432.3 | N.T | N.T |
| 165 | 11.4 | 114 | N.T | N.T |
| 166 | 61.6 | 207.9 | N.T | N.T |
| 167 | 0.39 | 381.3 | N.T | N.T |
| 168 | 5.23 | 65.6 | N.T | N.T |
| 169 | 2.33 | 310 | N.T | N.T |
| 170 | 1.28 | 27.6 | N.T | 27.5 |
| 171 | 0.54 | 14.5 | N.T | 12.3 |
| 172 | 1.71 | 1284 | N.T | N.T |
| 173 | 2.23 | 613.4 | N.T | N.T |
| 174 | 8.67 | 4169 | N.T | N.T |
| 176 | 1.88 | 245.3 | N.T | 497 |
| 177 | 0.74 | 638.9 | 2032 | N.T |
| 178 | 4.21 | 905.9 | N.T | N.T |
| 179 | 3.66 | 2.75 | N.T | 4.44 |
| 180 | 3.63 | 5042 | N.T | N.T |
| 181 | 2.93 | 13.1 | N.T | 28.4 |
| 182 | 9.29 | 4.36 | N.T | 7.04 |
| 183 | 1.49 | 741.6 | N.T | N.T |
| 184 | 10.2 | 8.97 | N.T | 15.9 |
| 185 | 6.16 | 274.6 | N.T | N.T |
| 186 | 23.5 | 85 | N.T | N.T |
| 187 | 4.89 | 14.8 | N.T | 20.9 |
| 188 | 1.74 | 29.7 | N.T | 80.4 |
| 189 | 6.31 | 39.2 | N.T | 27.8 |
| 190 | 7.33 | 93.4 | N.T | N.T |
| 191 | 3.38 | 24.6 | N.T | 25.9 |
| 192 | 1.98 | 28 | N.T | 82.1 |
| 193 | 12.6 | 221 | N.T | N.T |
| 194 | 119.2 | 1029 | N.T | N.T |
| 195 | 52.4 | 1667 | N.T | N.T |
| 196 | 39.3 | 83.3 | N.T | N.T |
| 197 | 3.59 | 71.8 | N.T | 193 |
| 198 | 0.63 | 23.7 | N.T | 21.2 |
| 199 | 2.78 | 9.24 | N.T | 4.05 |
| 200 | 2.27 | 5.16 | N.T | 4.1 |
| 201 | 3.33 | 56.4 | N.T | 20.5 |
| 202 | 6.15 | 13.6 | N.T | 4.35 |
| 203 | 1.99 | 9.38 | N.T | 12.3 |
| 204 | 5.14 | 89.2 | N.T | N.T |
| 205 | 6.86 | 25.2 | N.T | 39.2 |
| 206 | 6.21 | 94.7 | N.T | 106 |
| 207 | 9.50 | 33.6 | N.T | 26.6 |
| 208 | 2.20 | 4.62 | N.T | 3.82 |
| 209 | 14.5 | 40.3 | N.T | N.T |
| 210 | 3.53 | 15.1 | N.T | 5.86 |
| 211 | 10.7 | 189 | N.T | N.T |
| 212 | 1.16 | 53.8 | N.T | N.T |
| 213 | 7.32 | 76.1 | N.T | N.T |
| 214 | 7.9 | 496 | N.T | N.T |
| 215 | 2.65 | 295 | N.T | N.T |
| 216 | 6.06 | 51.6 | N.T | N.T |
| 217 | 5.06 | 11.1 | N.T | 20.9 |
| 218 | 13.4 | 15.3 | N.T | 35.6 |
| 219 | 7.13 | 149 | N.T | N.T |
| 220 | 6.86 | 2287 | N.T | N.T |
| 221 | 16.8 | 19.3 | N.T | 29.0 |
| 222 | 6.42 | 35.8 | N.T | N.T |
| 223 | 3.23 | 4.56 | N.T | 16.8 |
| 224 | 24.5 | 305 | N.T | N.T |
| 225 | 8.51 | 78 | N.T | N.T |
| 226 | 2.31 | 99.2 | N.T | N.T |
| 227 | 7.36 | 24.1 | N.T | 109.2 |
| 228 | 91.7 | 84.8 | N.T | N.T |
| 229 | 9.36 | 529 | N.T | N.T |
| 230 | 4.67 | 77 | N.T | N.T |
| 231 | 1.31 | 1138 | N.T | N.T |
| 232 | 32.1 | 160 | N.T | N.T |
| 233 | 10.2 | 3.81 | N.T | 8.35 |
| 234 | 15.0 | 77.6 | N.T | N.T |
| 235 | 2.35 | 19.2 | N.T | N.T |
| 236 | 6.02 | 410 | N.T | N.T |
| 237 | 4.06 | 3.27 | N.T | 4.4 |
| 238 | 25.7 | 28.7 | N.T | 56.1 |
| 239 | 11.6 | 25.2 | N.T | N.T |
| 240 | 9.62 | 24.8 | N.T | 60.9 |
| 241 | 5.78 | 5.7 | N.T | 11.2 |
| 242 | 18.4 | 17.8 | N.T | N.T |
| 243 | 10.5 | 3.24 | N.T | 10.6 |
| 244 | 2.83 | 1.66 | N.T | 16.2 |
| 245 | 14.1 | 4.16 | N.T | 11.8 |
| 246 | 10.8 | 14.3 | N.T | N.T |
| 247 | 4.93 | 3.17 | N.T | 7.56 |
| 248 | 5.33 | 3.94 | N.T | 9.06 |
| 249 | 1.26 | 2.74 | N.T | 11.7 |
| 250 | 2.42 | 1.93 | N.T | N.T |
| 251 | 7.41 | 3.11 | N.T | 7.18 |
| 252 | 1.86 | 4.32 | N.T | N.T |
| 253 | 13.2 | 5.25 | N.T | N.T |
| 254 | 5.77 | 4.69 | N.T | N.T |
| 255 | 3.39 | 7.82 | N.T | 11.0 |
| 256 | 8.48 | 8.63 | N.T | N.T |
| 257 | 7.61 | 11.2 | N.T | N.T |
| 258 | 1.05 | 1.81 | N.T | 2.09 |
| 259 | 7.20 | 7.48 | N.T | N.T |
| 260 | 3.02 | 8.18 | N.T | 11.5 |
| 261 | 19.0 | 8.47 | N.T | N.T |
| 262 | 11.8 | 11.1 | N.T | N.T |
| 263 | 1.64 | 1.14 | N.T | 2.22 |
| 264 | 2.2 | 1.84 | N.T | 5.12 |
| 265 | 5.09 | 7.20 | N.T | 5.85 |
| 266 | 2.09 | 2.02 | N.T | 2.35 |
| 267 | 7.49 | 13.6 | N.T | N.T |
| 268 | 2.03 | 1.58 | N.T | 2.22 |
| 269 | 2.66 | 1.87 | N.T | N.T |
| 270 | 4.88 | 3.24 | N.T | N.T |
| 271 | 2.19 | 1.80 | N.T | N.T |
| 272 | 16.8 | 13.3 | N.T | N.T |
| CC-90011 | 3.1 | 8376 | >10000 | >10000 |
| SAHA(Vorinostat) | 709.7 | 473 | 1349 | 397.4 |

As can be seen from the data in Table I, the Example compounds of the present disclosure have significant inhibitory effects on both HDAC and LSD1. The present disclosure provides structurally novel compounds represented by formula (I), which have dual-target inhibitory effects on HDAC and LSD1, and the IC₅₀s for inhibiting the activity of both HDAC and LSD1 are below 2 μM, or less than 1 μM, or less than 100 nM, or even both less than 1 nM.

The data in Table II shows that the compounds of the present disclosure have significant inhibitory activity against small cell lung cancer cell H1417, small cell lung cancer cell H69, small cell lung cancer cell H526, or prostate cancer cell Du145, and have the potential to be used as medicaments for the treatment of these cancers.

1. A compound represented by formula (I) or a tautomer, stereoisomer, solvate, metabolite, isotopically-labeled compound, pharmaceutically acceptable salt or co-crystal thereof, 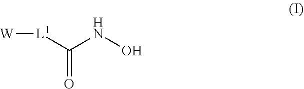
L¹ is selected from a bond, —C₁₋₁₀ alkyl-, —C₂₋₆ alkenyl-, —C₁₋₁₀ alkyl-C₂₋₆ alkenyl-, —C₂₋₆ alkynyl-, —C₆₋₁₀ heteroaryl-, —C₁₋₁₀ alkyl-(C₆₋₁₀ aryl or heteroaryl or heterocycloalkyl or heterocycloalkenyl)-C₂₋₆ alkenyl-, —(C₆₋₁₀ aryl or heteroaryl)-C₂₋₆ alkenyl-, —C₁₋₁₀ alkyl-(C₆₋₁₀ aryl)-, —C₁₋₁₀ alkyl-(C₆₋₁₀ aryl)-C₁₋₁₀ alkyl-, —NR^{a}—, —C₁₋₁₀ alkyl-(C₆₋₁₀ aryl)-C₂₋₆ alkynyl-, —C₁₋₁₀ alkyl-(C₆₋₁₀ heterocycloalkyl)-(C₆₋₁₀ aryl)-, —C₁₋₁₀ alkyl-NH-6-10-membered heteroaryl-, —C₁₋₁₀ alkyl-6-10-membered heteroaryl-, —C₁₋₁₀ alkyl-C₆₋₁₀ cycloalkenyl-C₂₋₆ alkenyl-, —C₁₋₁₀ alkyl-C₆₋₁₀ aryl-C₃₋₆ cycloalkenyl-, —C₁₋₁₀ alkyl-C₆₋₁₀ aryl-C₃₋₆ cycloalkyl-, —C₁₋₁₀ alkyl-O—C₆₋₁₀ aryl-, —C₁₋₁₀ alkyl-6-10-membered heteroaryl-C₁₋₁₀ alkyl-, —C₁₋₁₀ alkyl-6-10-membered aryl-O—C₁₋₁₀ alkyl-, —C₁₋₁₀ alkyl-6-10-membered heteroaryl-O—C₁₋₁₀ alkyl-, —C₁₋₁₀ alkyl-6-10-membered aryl-S—C₁₋₁₀ alkyl-, the alkyl, alkenyl, cycloalkyl, heterocycloalkyl, heterocycloalkenyl, aryl or heteroaryl is optionally substituted with one or more substituents selected from hydrogen, halogen, CN, CF₃, hydroxyl, C₁₋₆ alkyl, C₁₋₆ alkoxy, C₂₋₆ alkenyl, C₂₋₆ alkynyl, —NR^{a}R^{b}, COOH, —C(═O)NR^{a}R^{b}, the heteroaryl, heterocycloalkyl contain 1 to 4 heteroatom selected from N, O, or S; alternatively, one or more alkyl groups of the alkyl may optionally be replaced by one or more groups selected from —C(═O)—, —S(═O)₂— or —NR^{a}—;
W is selected from: 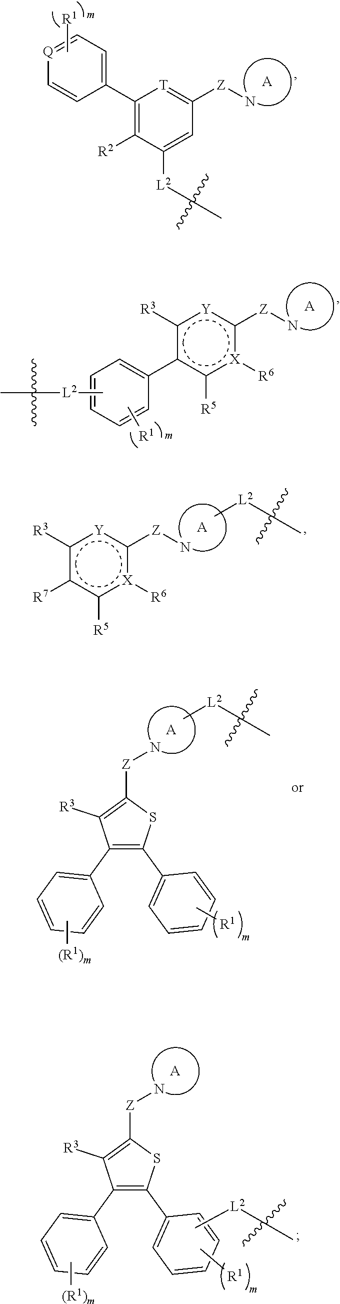
L² is selected from a bond, —O—, —C(═O)—, —NR^{a}—, —CH₂—NR^{a}—, —NR^{a}—C(O)—, —NR^{a}—S(═O)₂—, —S— or —S(═O)₂—;
ring A is selected from nitrogen-containing C₃₋₁₀ heteroaryl, C₃₋₁₀ heterocycloalkyl or C₃₋₁₀ heterocycloalkenyl, wherein, the heteroaryl, heterocycloalkyl or heterocycloalkenyl is optionally substituted with one or more R⁴, the heteroaryl, heterocycloalkyl contain 1 to 4 heteroatoms optionally selected from N, O or S;
R⁴ is each independently selected at each occurrence from hydrogen, halogen, CN, hydroxyl, C₁₋₆ alkyl, C₁₋₆ alkoxy, C₂₋₆ alkenyl, C₂₋₆ alkynyl, COOH, —NR^{a}R^{b}, —C(═O)NR^{a}R^{b}; optionally, when R⁴ is selected from C₁₋₆ alkyl, any two R⁴ and the atom to which they connect can collectively form a 5 to 10-membered heteroalicyclic;
R¹, R⁶ are each independently selected at each occurrence from hydrogen, halogen, CN, hydroxyl, C₁₋₆ alkyl, C₁₋₆ alkyl-CN, C₁₋₆ alkoxy, hydroxyl-substituted C₁₋₆ alkyl, halogen-substituted C₁₋₆ alkyl, halogen-substituted C₁₋₆ alkoxy, C₃₋₆ cycloalkyl, C₂₋₆ alkenyl, C₂₋₆ alkynyl, COOH, —NR^{a}R^{b}, —C(═O)NR^{a}R^{b}, —S(═O)₂R^{a} or —C₂₋₆ alkenyl-C(═O)NR^{a}R^{b};
R², R³, R⁷ are each independently selected from hydrogen, C₁₋₆ alkyl, C₁₋₆ alkoxy, C₆₋₁₀ aryl or C₆₋₁₀ heteroaryl, and R³ and R⁷ are not both hydrogen, wherein, the aryl or heteroaryl is optionally substituted with one or more substituents selected from hydrogen, halogen, CN, CF₃, hydroxyl, C₁₋₆ alkyl, C₁₋₆ alkoxy, C₂₋₆ alkenyl, C₂₋₆ alkynyl, C₃₋₆ cycloalkyl, halogen-substituted C₁₋₆ alkoxy, halogen-substituted C₁₋₆ alkyl, hydroxyl-substituted C₁₋₆ alkyl, hydroxyl-substituted C₁₋₆ alkoxy, hydroxyl-substituted C₁₋₆ alkoxy-C₃₋₆ cycloalkyl, COOH, —NR^{a}R^{b}, —S(═O)₂R^{a}, —C(═O)NR^{a}R^{b}, —C₂₋₆ alkenyl-C(═O)NR^{a}R^{b}, 3- to 6-membered heterocycloalkyl, heterocycloalkenyl, the heteroaryl, heterocycloalkyl contain 1 to 4 heteroatoms optionally selected from N, O or S;
m is selected from 0, 1, 2, 3, 4 or 5;
Q, T are each independently selected from N or C;
X, Y are each independently selected from C and N;
Z is selected from a bond, —CH₂—, —C(═O) or —S(═O)₂—;
R⁵ is each independently selected at each occurrence from hydrogen, halogen, CN, hydroxyl, C₁₋₆ alkyl, C₁₋₆ alkoxy, C₂₋₆ alkenyl, C₂₋₆ alkynyl, ═O, COOH, —NR^{a}R^{b}, —C(═O)NR^{a}R^{b}, C₃₋₆ cycloalkyl, 3- to 6-membered heterocycloalkyl, C₆₋₁₀ aryl or C₆₋₁₀ heteroaryl, wherein, the alkyl, alkoxy, alkenyl, alkynyl, cycloalkyl, heterocycloalkyl, aryl or heteroaryl is optionally substituted with one or more substituents selected from hydrogen, halogen, CN, NO₂, CF₃, CHF₂, hydroxyl, C₁₋₆ alkyl, C₁₋₆ alkoxy, —C(═O)—C₁₋₆ alkoxy, C₂₋₆ alkenyl, C₂₋₆ alkynyl, COOH, —NR^{a}R^{b}, —C(—O)NR^{a}R^{b}, the heteroaryl, heterocycloalkyl contain 1 to 4 heteroatoms optionally selected from N, O or S;
alternatively, R⁵, R⁶ and the atom to which both of them directly connect collectively form cycloalkyl, heteroalicyclic, aryl or heteroaryl, wherein the cycloalkyl, heteroalicyclic, aryl or heteroaryl is optionally substituted with one or more substituents selected from hydrogen, halogen, CN, hydroxyl, C₁₋₆ alkyl, C₁₋₆ alkoxy, C₂₋₆ alkenyl, C₂₋₆ alkynyl, —NR^{a}R^{b}, —C(═O)NR^{a}R^{b};
R^{a}, R^{b} are each independently selected at each occurrence from hydrogen, hydroxyl, C₁₋₆ alkyl, C₁₋₆ alkoxy, C₂₋₆ alkenyl, C₂₋₆ alkynyl, C₃₋₆ cycloalkyl, halogen-substituted C₁₋₆ alkyl, 3- to 6-membered heterocycloalkyl, C₆₋₁₀ aryl or C₆₋₁₀ heteroaryl, the heteroaryl, heterocycloalkyl contain 1 to 4 heteroatoms optionally selected from N, O or S; [symbol]
indicates a double bond may be present or not present at any position within the ring.

2. (canceled)

3. The compound or tautomer, stereoisomer, solvate, metabolite, isotopically-labeled compound, pharmaceutically acceptable salt or co-crystal thereof according to claim 1, wherein, the compound is represented by formula (II-1): 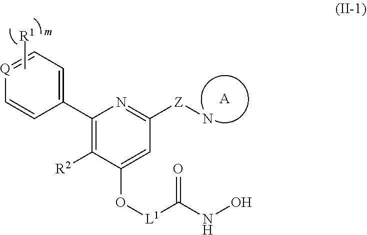
wherein,
L¹ is selected from —C₁₋₁₀ alkyl-, —C₂₋₆ alkenyl-, —C₁₋₁₀ alkyl-C₂₋₆ alkenyl-, —C₂₋₆ alkynyl-, —C₁₋₁₀ alkyl-(C₆₋₁₀ aryl)-C₂₋₆ alkenyl-, the alkyl, alkenyl are optionally substituted with one or more substituents selected from hydrogen, halogen, CN, CF₃, hydroxyl, C₁₋₆ alkyl, C₁₋₆ alkoxy;
preferably, L¹ is selected from —C₁₋₁₀ alkyl-, —C₁₋₁₀ alkyl-C₂₋₆ alkenyl-, —C₁₋₁₀ alkyl-(C₆₋₁₀ aryl)-C₂₋₆ alkenyl-;
preferably, L¹ is selected from —C₁₋₁₀ alkyl-, —C₁₋₁₀ alkyl-(C₆₋₁₀ aryl)-C₂₋₆ alkenyl-;
preferably, L¹ is selected from —C₁₋₆ alkyl-, —C₁₋₆ alkyl-phenylene-C₂₋₆ alkenyl-.

4. The compound or tautomer, stereoisomer, solvate, metabolite, isotopically-labeled compound, pharmaceutically acceptable salt or co-crystal thereof according to claim 3, wherein,
ring A is selected from nitrogen-containing C₃₋₁₀ heteroaryl or C₃₋₁₀ heterocycloalkyl, wherein, the heteroaryl, heterocycloalkyl are optionally substituted with one or more R⁴;
preferably, ring A is selected from nitrogen-containing C₃₋₁₀ heterocycloalkyl, wherein, the heterocycloalkyl is optionally substituted with one or more R⁴;
preferably, ring A is selected from: 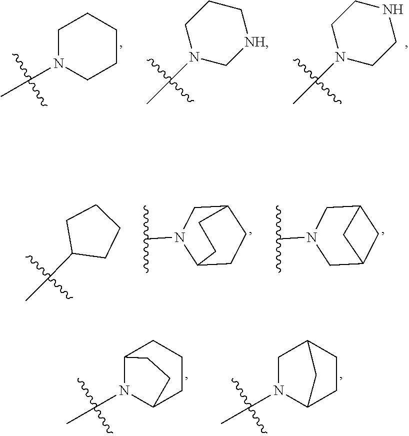
wherein the 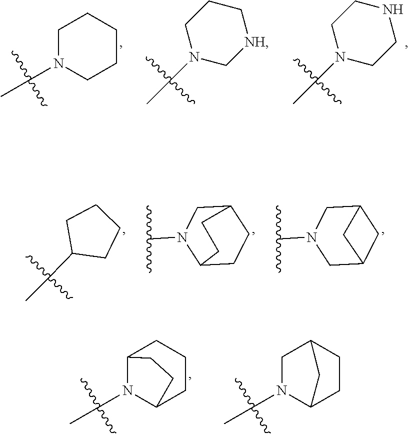
are optionally substituted with R⁴;
preferably, ring A is selected from: 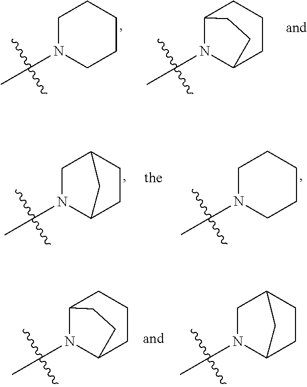 [SMILES: *N1CCCCC1 ; *N1C2CCCC1CC2 ; *N1CC2CCC1C2 ; *N1CCCCC1 ; *N1C2CCCC1CC2 ; *N1CC2CCC1C2]
are optionally substituted with R⁴;
preferably R⁴ is each independently selected at each occurrence from hydrogen, halogen, CN, hydroxyl, C₁₋₆ alkyl, C₁₋₆ alkoxy, C₂₋₆ alkenyl, C₂₋₆ alkynyl, COOH, —NR^{a}R^{b}, —C(═O)NR^{a}R^{b};
preferably, R⁴ is each independently selected at each occurrence from hydrogen, halogen, CN, hydroxyl, C₁₋₆ alkyl, C₁₋₆ alkoxy, —NR^{a}R^{b};
preferably, R⁴ is selected from hydrogen, —NR^{a}R^{b}, C₁₋₆ alkyl;
preferably, R⁴ is selected from hydrogen, —NR^{a}R^{b}.

5. (canceled)

6. The compound or tautomer, stereoisomer, solvate, metabolite, isotopically-labeled compound, pharmaceutically acceptable salt or co-crystal thereof according to claim 3, wherein,
R¹ is selected from hydrogen, halogen, CN, hydroxyl, C₁₋₆ alkyl, C₁₋₆ alkoxy, hydroxyl-substituted C₁₋₆ alkyl, halogen-substituted C₁₋₆ alkyl, halogen-substituted C₁₋₆ alkoxy, C₃₋₆ cycloalkyl, C₂₋₆ alkenyl, C₂₋₆ alkynyl;
preferably, R¹ is selected from hydrogen, halogen, CN, C₁₋₆ alkyl;
preferably, R¹ is selected from hydrogen, halogen, CN;
R² is selected from hydrogen, halogen, CN, hydroxyl, C₁₋₆ alkyl, C₁₋₆ alkoxy, C₂₋₆ alkenyl, C₂₋₆ alkynyl, COOH, —NR^{a}R^{b}, C₆₋₁₀ aryl or C₆₋₁₀ heteroaryl, wherein, the alkyl, alkoxy, alkenyl, aryl or heteroaryl is optionally substituted with one or more substituents selected from hydrogen, halogen, CN, hydroxyl, C₁₋₆ alkyl, C₁₋₆ alkoxy, C₂₋₆ alkenyl, C₂₋₆ alkynyl, COOH, —NR^{a}R^{b}, —S(═O)₂R^{a}, —O—C₁₋₆ alkyl-OH, the heteroaryl, heterocycloalkyl contain 1 to 4 heteroatoms optionally selected from N, O or S;
preferably, R² is selected from hydrogen, C₆₋₁₀ aryl or C₆₋₁₀ heteroaryl, wherein, the aryl or heteroaryl is optionally substituted with one or more substituents selected from hydrogen, halogen, hydroxyl, C₁₋₆ alkyl, C₁₋₆ alkoxy, —NR^{a}R^{b}, —S(═O)₂R^{a}, —O—C₁₋₆ alkyl-OH, the heteroaryl, heterocycloalkyl contain 1 to 4 heteroatoms optionally selected from N, O or S;
preferably, R² is selected from hydrogen, 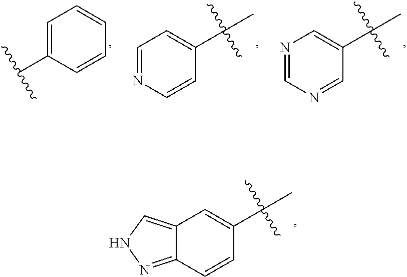
wherein, the 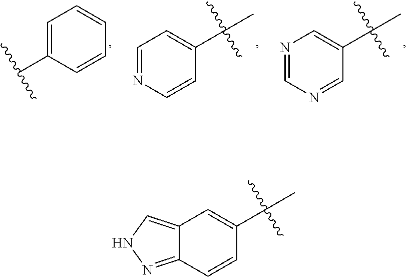
are optionally substituted with one or more substituents selected from hydrogen, halogen, hydroxyl, C₁₋₆ alkyl, C₁₋₆ alkoxy, —NR^{a}R^{b}, —S(═O)₂—R^{a}, —O—C₁₋₆ alkyl-OH;
preferably, R² is selected from 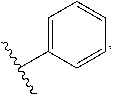
wherein, the 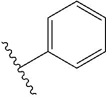 [SMILES: *c1ccccc1]
is optionally substituted with one or more substituents selected from hydrogen, hydroxyl, C₁₋₆ alkoxy.

7. The compound or tautomer, stereoisomer, solvate, metabolite, isotopically-labeled compound, pharmaceutically acceptable salt or co-crystal thereof according to claim 3, wherein,
m is selected from 0, 1, 2 or 3; preferably, m is selected from 1 or 2; more preferably, m is 2;
Q is each independently selected from N or C; preferably, Q is selected from C;
preferably R^{a}, R^{b} are each independently selected at each occurrence from hydrogen, hydroxyl, C₁₋₆ alkyl, C₁₋₆ alkoxy, C₂₋₆ alkenyl, C₂₋₆ alkynyl, C₃₋₆ cycloalkyl, halogen-substituted C₁₋₆ alkyl;
preferably, R^{a}, R^{b} are each independently selected at each occurrence from hydrogen, hydroxyl, C₁₋₆ alkyl, C₁₋₆ alkoxy, C₂₋₆ alkenyl, C₂₋₆ alkynyl, C₃₋₆ cycloalkyl;
preferably, R^{a}, R^{b} are each independently selected at each occurrence from hydrogen and methyl;
preferably, R^{a}, R^{b} are each independently selected from hydrogen.

8. (canceled)

9. The compound or tautomer, stereoisomer, solvate, metabolite, isotopically-labeled compound, pharmaceutically acceptable salt or co-crystal thereof according to claim 1, wherein, the compound is represented by formula (III-1): 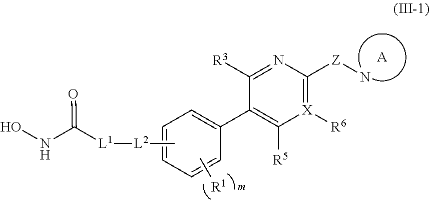
wherein,
X is selected from C and N;
L¹ is selected from a bond, —C₁₋₁₀ alkyl-, —C₂₋₆ alkenyl-, —C₁₋₁₀ alkyl-C₂₋₆ alkenyl-, —C₂₋₆ alkynyl-, —C₁₋₁₀ alkyl-(C₆₋₁₀ aryl or heteroaryl)-C₂₋₆ alkenyl-, —C₁₋₁₀ alkyl-(C₆₋₁₀ aryl)-, —NR^{a}—, the alkyl, alkoxy, alkenyl, aryl or heteroaryl is optionally substituted with one or more substituents selected from hydrogen, halogen, CN, CF₃, hydroxyl, C₁₋₆ alkyl, C₁₋₆ alkoxy, C₂₋₆ alkenyl, C₂₋₆ alkynyl, —NR^{a}R^{b}, COOH;
alternatively, one or more carbon atoms in the alkyl can optionally be replaced by one or more groups selected from —NH—;
preferably, L¹ is selected from a bond, —C₁₋₁₀ alkyl-, —C₂₋₆ alkenyl-, —C₁₋₁₀ alkyl-C₂₋₆ alkenyl-, —C₁₋₁₀ alkyl-(C₆₋₁₀ aryl or heteroaryl)-C₂₋₆ alkenyl-, —C₁₋₁₀ alkyl-(C₆₋₁₀ aryl)-, the alkyl, alkoxy, alkenyl, aryl or heteroaryl is optionally substituted with one or more substituents selected from hydrogen, halogen, CN, CF₃, hydroxyl, C₁₋₆ alkoxy, C₂₋₆ alkenyl, —NR^{a}R^{b},
alternatively, one or more carbon atoms in the alkyl can optionally be replaced by one or more groups selected from —NH—;
preferably, L¹ is selected from —C₁₋₁₀ alkyl-, —C₁₋₁₀ alkyl-(C₆₋₁₀ aryl or heteroaryl)-C₂₋₆ alkenyl-, —C₁₋₁₀ alkyl-(C₆₋₁₀ aryl)-, the alkyl, aryl or heteroaryl is optionally substituted with one or more substituents selected from hydrogen, halogen, CN, CF₃, hydroxyl, C₁₋₆ alkyl, C₁₋₆ alkoxy.

10. The compound or tautomer, stereoisomer, solvate, metabolite, isotopically-labeled compound, pharmaceutically acceptable salt or co-crystal thereof according to claim 9, wherein,
L² is selected from a bond, —O—, —C(═O)—, —NR^{a}—, —NR^{a}—C(O)— or —S(═O)₂—;
preferably, L² is selected from a bond, —O—, —NR^{a}—;
preferably, L² is selected from —O—, —NR^{a}—.

11. The compound or tautomer, stereoisomer, solvate, metabolite, isotopically-labeled compound, pharmaceutically acceptable salt or co-crystal thereof according to claim 9, wherein,
ring A is selected from nitrogen-containing C₃₋₁₀ heteroaryl or C₃₋₁₀ heterocycloalkyl, wherein, the heteroaryl, heterocycloalkyl are optionally substituted with one or more R⁴;
preferably, ring A is selected from nitrogen-containing C₃₋₁₀ heterocycloalkyl, the C₃₋₁₀ heterocycloalkyl is optionally substituted with one or more R⁴;
preferably, ring A is selected from: 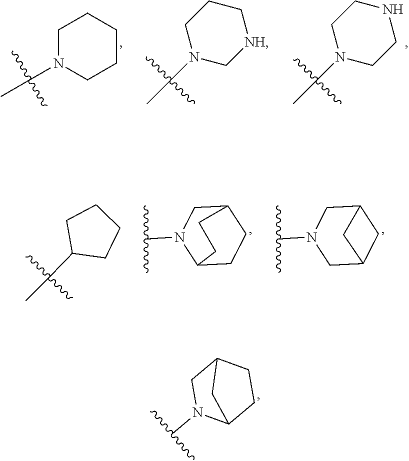
wherein, the 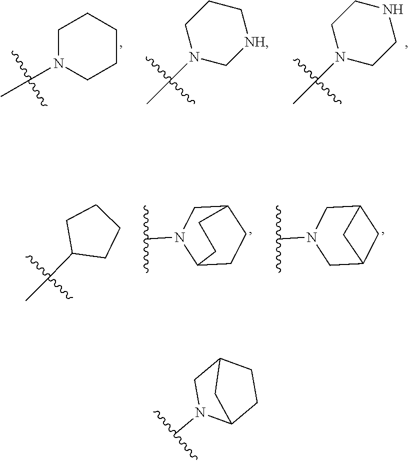
are optionally substituted with R⁴;
preferably, ring A is selected from: 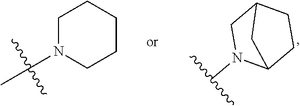 [SMILES: *N1CCCCC1 ; *N1CC2CCC1C2]
the 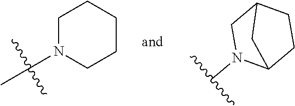 [SMILES: *N1CCCCC1 ; *N1CC2CCC1C2]
are optionally substituted with R⁴;
preferably R⁴ is each independently selected at each occurrence from hydrogen, halogen, CN, hydroxyl, C₁₋₆ alkyl, C₁₋₆ alkoxy, C₂₋₆ alkenyl, —NR^{a}R^{b};
preferably, R⁴ is selected from hydrogen, C₁₋₆ alkyl, —NR^{a}R^{b};
preferably, R⁴ is selected from hydrogen, —NR^{a}R^{b}.

12. (canceled)

13. The compound or tautomer, stereoisomer, solvate, metabolite, isotopically-labeled compound, pharmaceutically acceptable salt or co-crystal thereof according to claim 9, wherein,
R¹, R⁶ are each independently selected at each occurrence from hydrogen, halogen, CN, hydroxyl, C₁₋₆ alkyl, C₁₋₆ alkoxy, halogen-substituted C₁₋₆ alkyl, halogen-substituted C₁₋₆ alkoxy, C₃₋₆ cycloalkyl, C₂₋₆ alkenyl, C₂₋₆ alkynyl, COOH, —NR^{a}R^{b}, —C(═O)NR^{a}R^{b};
preferably, R¹, R⁶ are each independently selected at each occurrence from hydrogen, CN, hydroxyl, C₁₋₆ alkoxy;
preferably, R¹, R⁶ are each independently selected at each occurrence from hydrogen, CN, hydroxyl;
R³ is selected from hydrogen, C₁₋₆ alkyl, C₁₋₆ alkoxy, C₆₋₁₀ aryl or C₆₋₁₀ heteroaryl, wherein, the aryl or heteroaryl is optionally substituted with one or more substituents selected from hydrogen, halogen, CN, CF₃, hydroxyl, C₁₋₆ alkyl, C₁₋₆ alkoxy, C₂₋₆ alkenyl, the heteroaryl, heterocycloalkyl contain 1 to 4 heteroatoms optionally selected from N, O or S;
preferably, R³ is selected from hydrogen, C₁₋₆ alkyl, C₆₋₁₀ aryl or C₆₋₁₀ heteroaryl, wherein, the aryl or heteroaryl is optionally substituted with one or more substituents selected from hydrogen, halogen, CN, the heteroaryl, heterocycloalkyl contain 1 to 4 heteroatoms optionally selected from N, O or S;
preferably, R³ is selected from hydrogen, methyl, 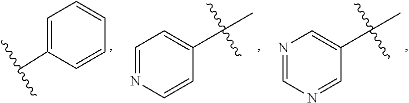
the 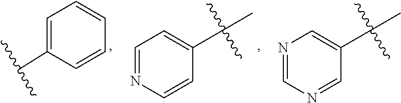
are optionally substituted with one or more substituents selected from hydrogen, halogen, CN;
preferably, R³ is selected from hydrogen, 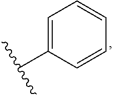 [SMILES: *c1ccccc1]
the 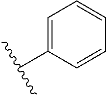 [SMILES: *c1ccccc1]
is optionally substituted with one or more substituents selected from hydrogen, halogen, CN.

14. The compound or tautomer, stereoisomer, solvate, metabolite, isotopically-labeled compound, pharmaceutically acceptable salt or co-crystal thereof according to claim 9, wherein,
R⁵ is each independently selected at each occurrence from hydrogen, halogen, CN, hydroxyl, C₁₋₆ alkyl, C₁₋₆ alkoxy, C₂₋₆ alkenyl, C₆₋₁₀ aryl or C₆₋₁₀ heteroaryl, wherein, the alkyl, alkoxy, alkenyl, aryl or heteroaryl is optionally substituted with one or more substituents selected from hydrogen, halogen, CN, CF₃, hydroxyl, C₁₋₆ alkyl, C₁₋₆ alkoxy, C₂₋₆ alkenyl, the heteroaryl, heterocycloalkyl contain 1 to 4 heteroatoms optionally selected from N, O or S;
alternatively, R⁵, R⁶ and the atom to which both of them directly connect collectively form cycloalkyl, heteroalicyclic, aryl or heteroaryl, wherein, the cycloalkyl, heteroalicyclic, aryl, heteroaryl are optionally substituted with one or more substituents selected from hydrogen, halogen, CN, hydroxyl, C₁₋₆ alkyl, C₁₋₆ alkoxy, C₂₋₆ alkenyl, C₂₋₆ alkynyl;
preferably, R⁵ is selected from hydroxyl, C₁₋₆ alkoxy, 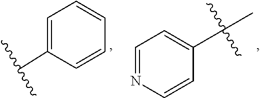
wherein, the alkoxy, 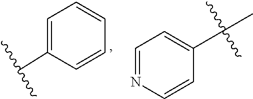
are optionally substituted with one or more substituents selected from hydrogen, halogen, CN, hydroxyl, C₁₋₆ alkyl, C₁₋₆ alkoxy;
alternatively, R⁵, R⁶ and the atom to which both of them directly connect collectively form 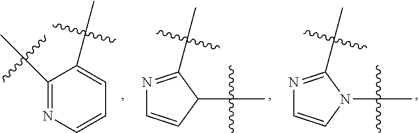
wherein, the 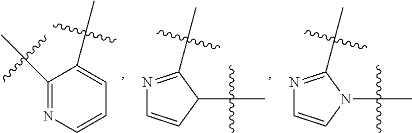
are optionally substituted with one or more groups selected from hydrogen, halogen, CN, hydroxyl, C₁₋₆ alkyl, C₁₋₆ alkoxy;
preferably, R⁵ is selected from 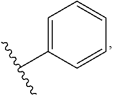
wherein, the 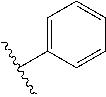 [SMILES: *c1ccccc1]
is optionally substituted with one or more substituents selected from hydrogen, halogen, CN;
alternatively, R⁵, R⁶ and the atom to which both of them directly connect collectively form

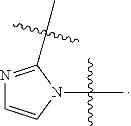 [SMILES: *c1nccn1*]

15. The compound or tautomer, stereoisomer, solvate, metabolite, isotopically-labeled compound, pharmaceutically acceptable salt or co-crystal thereof according to claim 9, wherein,
m is selected from 0, 1, 2 or 3; preferably, m is selected from 1 or 2;
Z is selected from a bond, —CH₂— or —C(═O); preferably, Z is selected from a bond;
R^{a}, R^{b} are each independently selected at each occurrence from hydrogen, halogen, CN, hydroxyl, C₁₋₆ alkyl, C₁₋₆ alkoxy, C₂₋₆ alkenyl, C₂₋₆ alkynyl, COOH, wherein, the alkyl, alkoxy, alkenyl, alkynyl are optionally substituted with one or more substituents selected from hydrogen, halogen, CN, CF₃, hydroxyl, C₁₋₆ alkyl, C₁₋₆ alkoxy, C₂₋₆ alkenyl, C₂₋₆ alkynyl, COOH;
preferably, R^{a}, R^{b} are each independently selected at each occurrence from hydrogen, C₁₋₆ alkyl;
preferably, R^{a}, R^{b} are each independently selected at each occurrence from hydrogen or methyl.

16. (canceled)

17. The compound or tautomer, stereoisomer, solvate, metabolite, isotopically-labeled compound, pharmaceutically acceptable salt or co-crystal thereof according to claim 1, wherein, the compound is represented by formula (IV-1a): 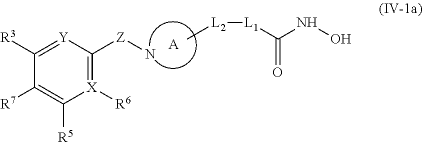
wherein,
L¹ is selected from a bond, —C₁₋₁₀ alkyl-, —C₂₋₆ alkenyl-, —C₁₋₁₀ alkyl-C₂₋₆ alkenyl-, —C₆₋₁₀ heteroaryl-, —C₁₋₁₀ alkyl-(C₆₋₁₀ aryl or heteroaryl or heterocycloalkyl or heterocycloalkenyl)-C₂₋₆ alkenyl-, —(C₆₋₁₀ aryl or heteroaryl)-C₂₋₆ alkenyl-, —C₁₋₁₀ alkyl-(C₆₋₁₀ aryl)-, —C₁₋₁₀ alkyl-(C₆₋₁₀ aryl)-C₁₋₁₀ alkyl-, —NR^{a}—, —C₁₋₁₀ alkyl-(C₆₋₁₀ aryl)-C₂₋₆ alkynyl-, —C₁₋₁₀ alkyl-(C₆₋₁₀ heterocycloalkyl)-(C₆₋₁₀ aryl)-, —C₁₋₁₀ alkyl-NH-6-10-membered heteroaryl-, —C₁₋₁₀ alkyl-6-10-membered heteroaryl-, —C₁₋₁₀ alkyl-C₆₋₁₀ cycloalkenyl-C₂₋₆ alkenyl-, —C₁₋₁₀ alkyl-C₆₋₁₀ aryl-C₃₋₆ cycloalkenyl-, —C₁₋₁₀ alkyl-C₆₋₁₀ aryl-C₃₋₆ cycloalkyl-, —C₁₋₁₀ alkyl-O—C₆₋₁₀ aryl-, —C₁₋₁₀ alkyl-6-10-membered heteroaryl-C₁₋₁₀ alkyl-, —C₁₋₁₀ alkyl-6-10-membered aryl-O—C₁₋₁₀ alkyl-, —C₁₋₁₀ alkyl-6-10-membered heteroaryl-O—C₁₋₁₀ alkyl-, —C₁₋₁₀ alkyl-6-10-membered aryl-S—C₁₋₁₀ alkyl-, the alkyl, alkenyl, cycloalkyl, heterocycloalkyl, heterocycloalkenyl, aryl or heteroaryl is optionally substituted with one or more substituents selected from hydrogen, halogen, CN, CF₃, hydroxyl, C₁₋₆ alkyl, C₁₋₆ alkoxy, C₂₋₆ alkenyl, the heteroaryl, heterocycloalkyl contain 1 to 4 heteroatoms optionally selected from N, O or S;
alternatively, one or more alkyl groups of the alkyl may optionally be replaced by one or more groups selected from —C(═O)—, —S(═O)₂— or —NR^{a}—;
preferably, L¹ is selected from —C₁₋₁₀ alkyl-, —C₂₋₆ alkenyl-, —C₆₋₁₀ heteroaryl-, —C₁₋₁₀ alkyl-(C₆₋₁₀ aryl or heteroaryl or heterocycloalkyl or heterocycloalkenyl)-C₂₋₆ alkenyl-, —(C₆₋₁₀ aryl or heteroaryl)-C₂₋₆ alkenyl-, —C₁₋₁₀ alkyl-(C₆₋₁₀ aryl)-, —C₁₋₁₀ alkyl-(C₆₋₁₀ aryl)-C₁₋₁₀ alkyl-, —C₁₋₁₀ alkyl-(C₆₋₁₀ aryl)-C₂₋₆ alkynyl-, —C₁₋₁₀ alkyl-(C₆₋₁₀ heterocycloalkyl)-(C₆₋₁₀ aryl)-, —C₁₋₁₀ alkyl-NH-6-10-membered heteroaryl-, —C₁₋₁₀ alkyl-6-10-membered heteroaryl-, —C₁₋₁₀ alkyl-C₆₋₁₀ cycloalkenyl-C₂₋₆ alkenyl-, —C₁₋₁₀ alkyl-C₆₋₁₀ aryl-C₃₋₆ cycloalkenyl-, —C₁₋₁₀ alkyl-C₆₋₁₀ aryl-C₃₋₆ cycloalkyl-, —C₁₋₁₀ alkyl-O—C₆₋₁₀ aryl-, —C₁₋₁₀ alkyl-6-10-membered heteroaryl-C₁₋₁₀ alkyl-, —C₁₋₁₀ alkyl-6-10-membered aryl-O—C₁₋₁₀ alkyl-, —C₁₋₁₀ alkyl-6-10-membered heteroaryl-O—C₁₋₁₀ alkyl-, —C₁₋₁₀ alkyl-6-10-membered aryl-S—C₁₋₁₀ alkyl-, the alkyl, alkenyl, cycloalkyl, heterocycloalkyl, heterocycloalkenyl, aryl or heteroaryl is optionally substituted with one or more substituents selected from hydrogen, halogen, CN, CF₃, hydroxyl, C₁₋₆ alkyl, C₁₋₆ alkoxy, C₂₋₆ alkenyl, the heteroaryl, heterocycloalkyl contain 1 to 4 heteroatoms optionally selected from N, O or S;
preferably, L¹ is selected from —C₁₋₁₀ alkyl-, —C₁₋₁₀ alkyl-C₂₋₆ alkenyl-, —C₆₋₁₀ heteroaryl-, —C₁₋₁₀ alkyl-(C₆₋₁₀ aryl or heteroaryl)-C₂₋₆ alkenyl-, —(C₆₋₁₀ aryl or heteroaryl)-C₂₋₆ alkenyl-, —C₁₋₁₀ alkyl-(C₆₋₁₀ aryl)-, —C₁₋₁₀ alkyl-(C₆₋₁₀ aryl)-C₁₋₁₀ alkyl-, —C₁₋₁₀ alkyl-(C₆₋₁₀ heterocycloalkyl)-(C₆₋₁₀ aryl)-, the alkyl, alkenyl, cycloalkyl, heterocycloalkyl, heterocycloalkenyl, aryl or heteroaryl is optionally substituted with one or more substituents selected from hydrogen, halogen, CN, CF₃, hydroxyl, C₁₋₆ alkyl, C₁₋₆ alkoxy, C₂₋₆ alkenyl, the heteroaryl, heterocycloalkyl contain 1 to 4 heteroatoms optionally selected from N, O or S;
preferably, L¹ is selected from —C₁₋₁₀ alkyl-, —C₁₋₁₀ alkyl-C₂₋₆ alkenyl-, —C₆₋₁₀ heteroaryl-, —C₁₋₁₀ alkyl-(C₆₋₁₀ aryl or heteroaryl)-C₂₋₆ alkenyl-, —(C₆₋₁₀ aryl or heteroaryl)-C₂₋₆ alkenyl-, —C₁₋₁₀ alkyl-(C₆₋₁₀ aryl)-, —C₁₋₁₀ alkyl-(C₆₋₁₀ aryl)-C₁₋₁₀ alkyl-, —C₁₋₁₀ alkyl-(C₆₋₁₀ heterocycloalkyl)-(C₆₋₁₀ aryl)-, the alkyl, alkenyl, cycloalkyl, heterocycloalkyl, heterocycloalkenyl, aryl or heteroaryl is optionally substituted with one or more substituents selected from hydrogen, halogen, CN, CF₃, hydroxyl, C₁₋₆ alkyl, C₁₋₆ alkoxy, C₂₋₆ alkenyl, the heteroaryl, heterocycloalkyl contain 1 to 4 heteroatoms optionally selected from N, O or S;
preferably, L¹ is selected from —C₁₋₁₀ alkyl-(C₆₋₁₀ aryl or heteroaryl)-C₂₋₆ alkenyl-, the alkyl, aryl or heteroaryl is optionally substituted with one or more substituents selected from hydrogen, halogen, CN, CF₃, hydroxyl, C₁₋₆ alkyl, C₁₋₆ alkoxy;
preferably, L¹ is selected from —CH₂—, —CH₂—(C═C)—, —(CH₂)₄—, —(CH₂)₆—, —(C═O)-phenyl-(C═C)—, —CH₂-phenyl-, —(CH₂)₃-phenyl-, —CH₂-phenyl-(CH₂)₂—, —(CH₂)₂-phenyl-CH₂—, —CH₂-phenyl-(C═C)—, —(CH₂)₂-phenyl-(C═C)—, —CH₂-phenyl-(C≡C)—, —CH₂-phenyl-(C═C)—CH₂—, -phenyl-(C═C)—, pyrimidinyl, 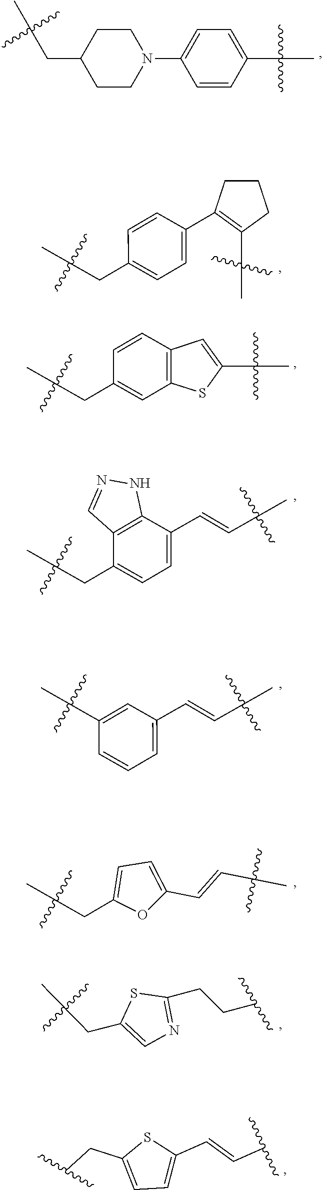 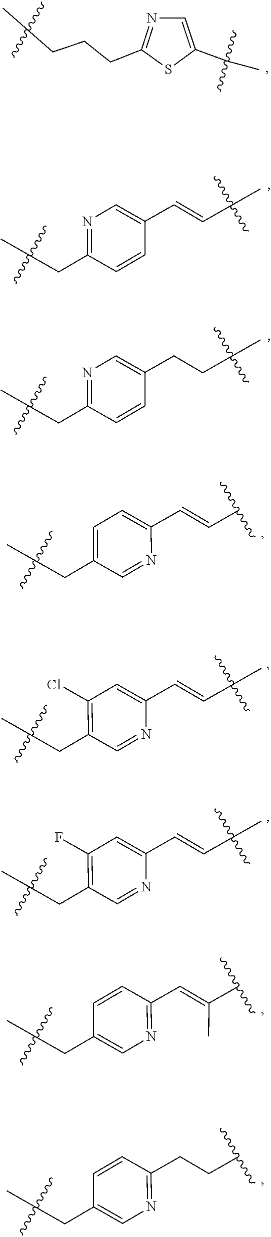 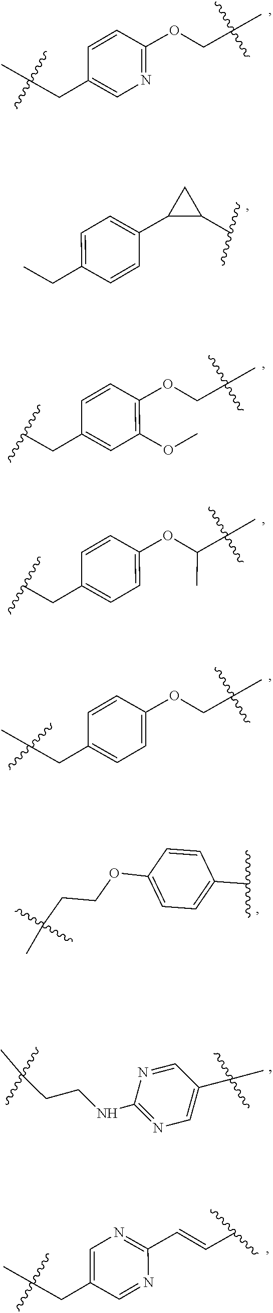 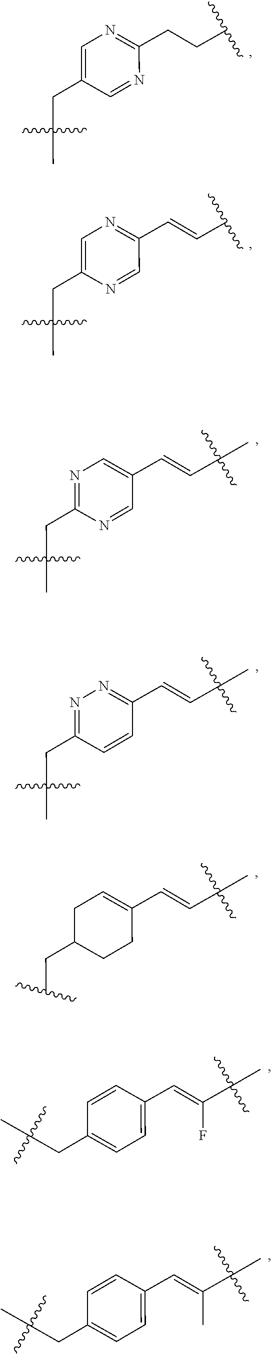 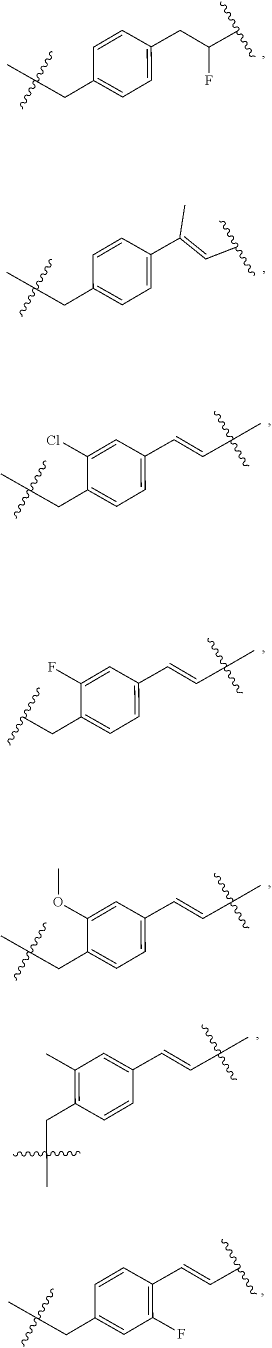 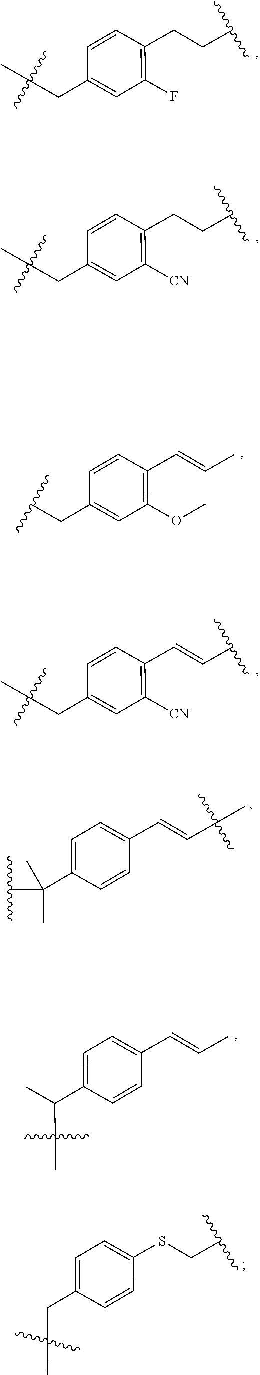
preferably, L¹ is selected from

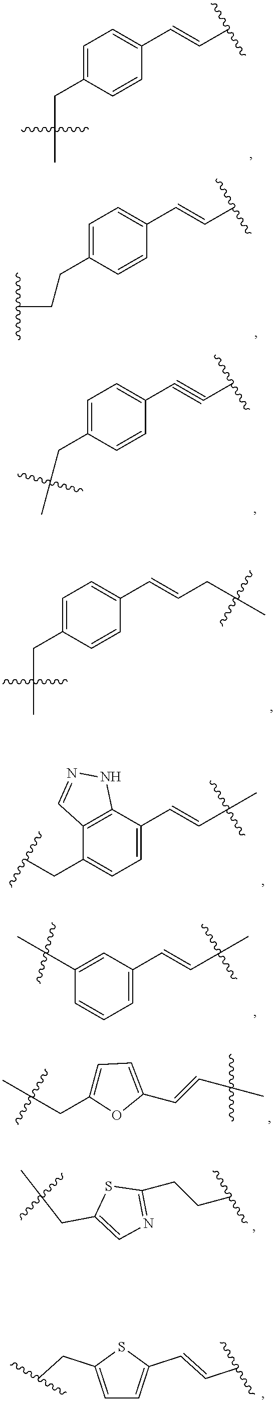

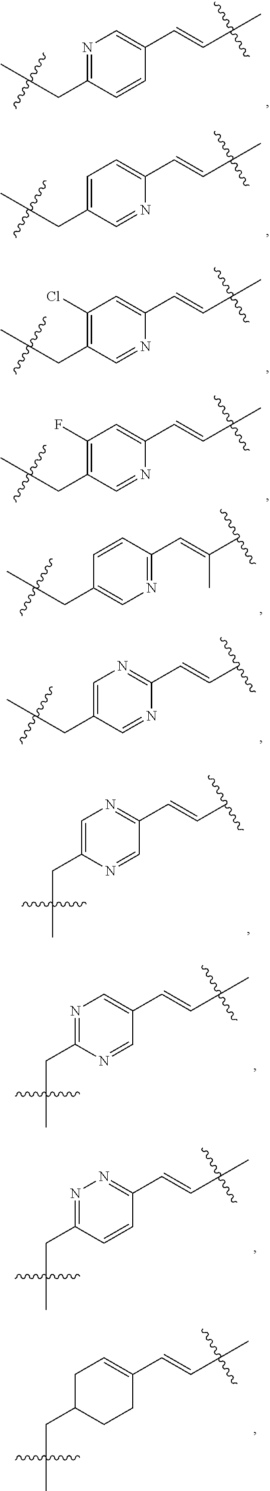

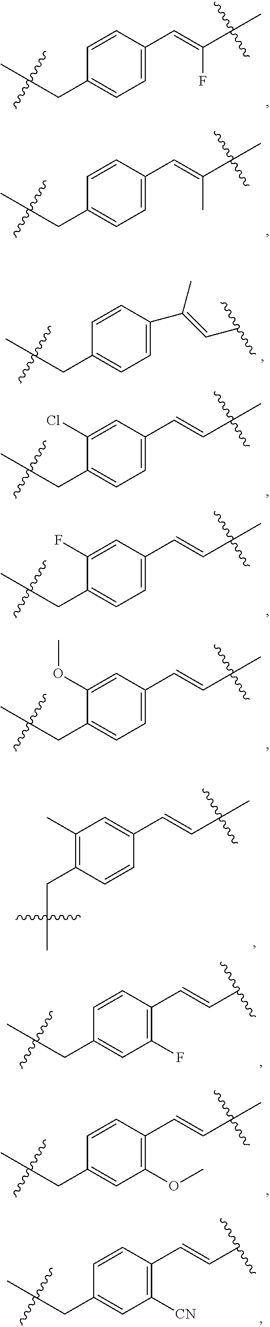

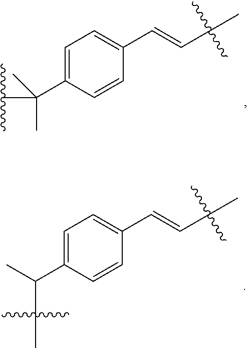 [SMILES: */C=C/c1ccc(C(*)(C)C)cc1 ; */C=C/c1ccc(C(*)C)cc1]

18. The compound or tautomer, stereoisomer, solvate, metabolite, isotopically-labeled compound, pharmaceutically acceptable salt or co-crystal thereof according to claim 17, wherein,
L² is selected from a bond, —O—, —C(═O)—, —S—, —NR^{a}—, —CH₂—NR^{a}—, —NR^{a}—C(═O) or —NR^{a}—S(═O)₂—;
preferably, L² is selected from a bond, —NR^{a}—, —CH₂—NR^{a}—, —NR^{a}—C(═O) or —NR^{a}—S(═O)₂—;
preferably, L² is selected from —NR^{a}—, —NR^{a}—C(═O) or —NR^{a}—S(═O)₂—;
preferably, L² is selected from a bond, —C(═O)— or —NR^{a}—;
preferably, L² is selected from —NR^{a}-.

19. The compound or tautomer, stereoisomer, solvate, metabolite, isotopically-labeled compound, pharmaceutically acceptable salt or co-crystal thereof according to claim 17, wherein,
ring A is selected from nitrogen-containing C₃₋₁₀ heteroaryl or C₃₋₁₀ heterocycloalkyl, wherein, the heteroaryl, heterocycloalkyl are optionally substituted with one or more R⁴; the heteroaryl, heterocycloalkyl contain 1 to 4 heteroatoms optionally selected from N, O or S;
preferably, ring A is selected from C₃₋₁₀ heterocycloalkyl, wherein, the heterocycloalkyl is optionally substituted with one or more R⁴;
preferably, ring A is selected from: 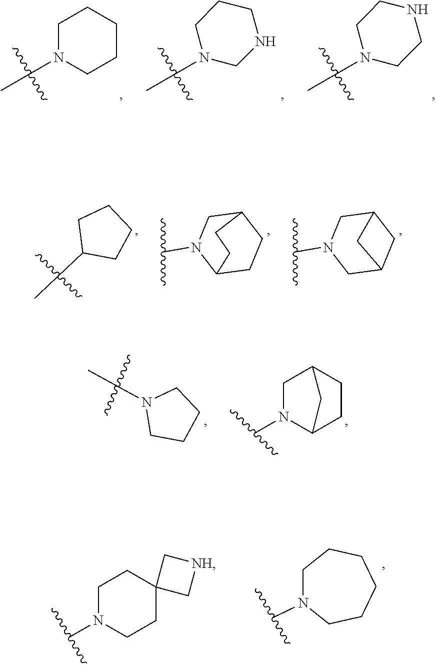
wherein, the 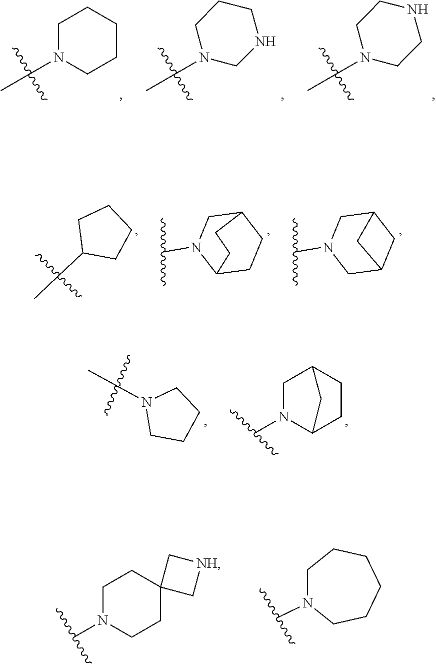
are optionally substituted with R⁴,
preferably, ring A is selected from: 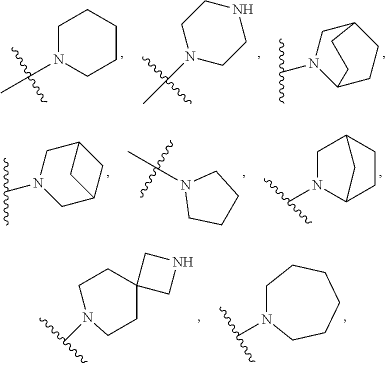
wherein, the 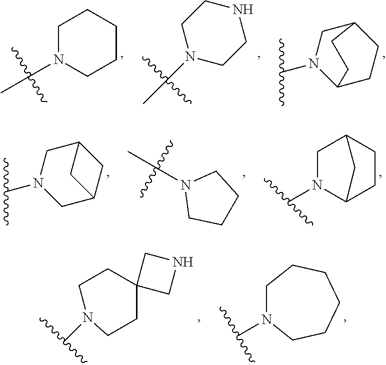
are optionally substituted with R⁴;
preferably, ring A is selected from: 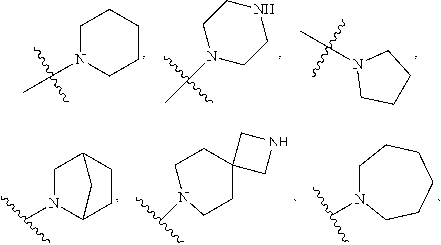
wherein, the 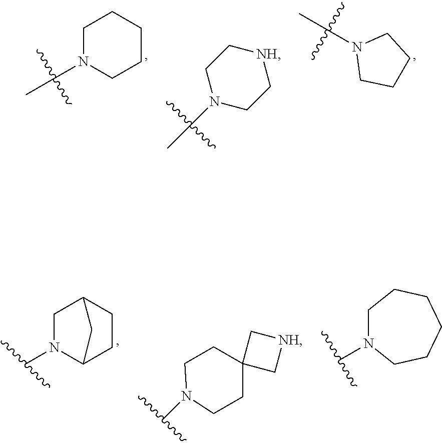 [SMILES: *N1CCCCC1 ; *N1CCNCC1 ; *N1CCCC1 ; *N1CC2CCC1C2 ; *N1CCC2(CC1)CNC2 ; *N1CCCCCC1]
are optionally substituted with R⁴;
preferably, ring A is selected from: 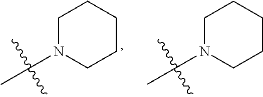 [SMILES: *N1CCCCC1 ; *N1CCCCC1]
is optionally substituted with R⁴;
preferably R⁴ is each independently selected at each occurrence from hydrogen, halogen, CN, hydroxyl, C₁₋₆ alkyl, C₁₋₆ alkoxy, C₂₋₆ alkenyl, —NR^{a}R^{b};
preferably, R⁴ is selected from hydrogen, C₁₋₆ alkyl, —NR^{a}R^{b};
preferably, R⁴ is selected from hydrogen, C₁₋₆ alkyl.

20. (canceled)

21. The compound or tautomer, stereoisomer, solvate, metabolite, isotopically-labeled compound, pharmaceutically acceptable salt or co-crystal thereof according to claim 17, wherein,
R⁶ is each independently selected at each occurrence from hydrogen, halogen, CN, hydroxyl, C₁₋₆ alkyl, C₁₋₆ alkyl-CN, halogen-substituted C₁₋₆ alkyl, C₁₋₆ alkoxy, C₃₋₆ cycloalkyl, C₂₋₆ alkenyl, C₂₋₆ alkynyl, COOH, —NR^{a}R^{b}, —C(═O)NR^{a}R^{b};
preferably, R⁶ is each independently selected at each occurrence from hydrogen, halogen, CH₂—CN, CN, C₁₋₆ alkyl, C₁₋₆ alkoxy, halogen-substituted C₁₋₆ alkyl;
preferably, R⁶ is each independently selected at each occurrence from hydrogen, halogen, CH₂—CN, CN, C₁₋₆ alkyl;
preferably, R⁶ is each independently selected at each occurrence from hydrogen, CN;
R³, R⁷ are each independently selected from hydrogen, C₁₋₆ alkyl, C₁₋₆ alkoxy, C₆₋₁₀ aryl or C₆₋₁₀ heteroaryl, and R³ and Rare not both hydrogen, wherein, the aryl or heteroaryl is optionally substituted with one or more substituents selected from hydrogen, halogen, CN, CF₃, hydroxyl, C₁₋₆ alkyl, C₁₋₆ alkoxy, C₂₋₆ alkenyl, C₂₋₆ alkynyl, C₃₋₆ cycloalkyl, halogen-substituted C₁₋₆ alkoxy, halogen-substituted C₁₋₆ alkyl, hydroxyl-substituted C₁₋₆ alkyl, hydroxyl-substituted C₁₋₆ alkoxy, COOH, —NR^{a}R^{b}, —S(═O)₂R^{a}, —C(═O)NR^{a}R^{b}, 3- to 6-membered heterocycloalkyl, hydroxyl-substituted C₁₋₆ alkoxy-C₃₋₆ cycloalkyl, heterocycloalkenyl, the heteroaryl, heterocycloalkyl contain 1 to 4 heteroatoms optionally selected from N, O or S;
preferably, R³, R⁷ are each independently selected from hydrogen, C₁₋₆ alkyl, C₁₋₆ alkoxy, C₆₋₁₀ aryl or C₆₋₁₀ heteroaryl, and R³ and R⁷ are not both hydrogen, wherein, the aryl or heteroaryl is optionally substituted with one or more substituents selected from hydrogen, halogen, CN, hydroxyl, C₁₋₆ alkyl, C₁₋₆ alkoxy, heterocycloalkenyl, the heteroaryl, heterocycloalkyl contain 1 to 4 heteroatoms optionally selected from N, O or S;
preferably, R³, R⁷ are each independently selected from hydrogen, C₁₋₆ alkyl, C₁₋₆ alkoxy, C₆₋₁₀ aryl or C₆₋₁₀ heteroaryl, and R³ and R⁷ are not both hydrogen, wherein, the aryl or heteroaryl is optionally substituted with one or more substituents selected from hydrogen, halogen, CN, CF₃, hydroxyl, C₁₋₆ alkyl, C₁₋₆ alkoxy, C₂₋₆ alkenyl, C₂₋₆ alkynyl, halogen-substituted C₁₋₆ alkoxy, halogen-substituted C₁₋₆ alkyl, hydroxyl-substituted C₁₋₆ alkoxy, hydroxyl-substituted C₁₋₆ alkoxy-C₃₋₆ cycloalkyl, COOH, —NR^{a}R^{b}, —S(═O)₂R^{a}, —C(═O)NR^{a}R^{b}, 3- to 6-membered heterocycloalkyl, heterocycloalkenyl, the heteroaryl, heterocycloalkyl contain 1 to 4 heteroatoms optionally selected from N, O or S;
preferably, R³, R⁷ are selected from hydrogen, 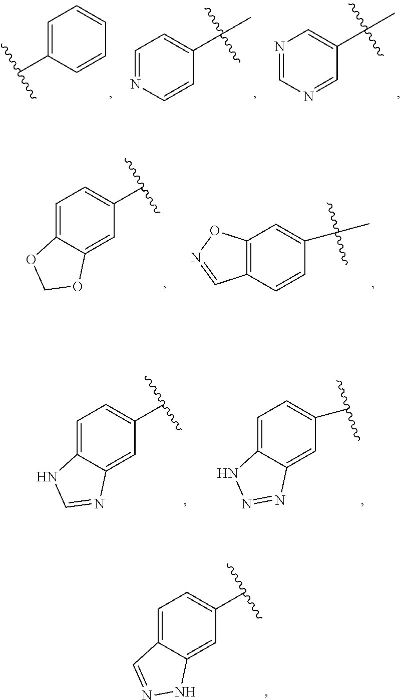
the 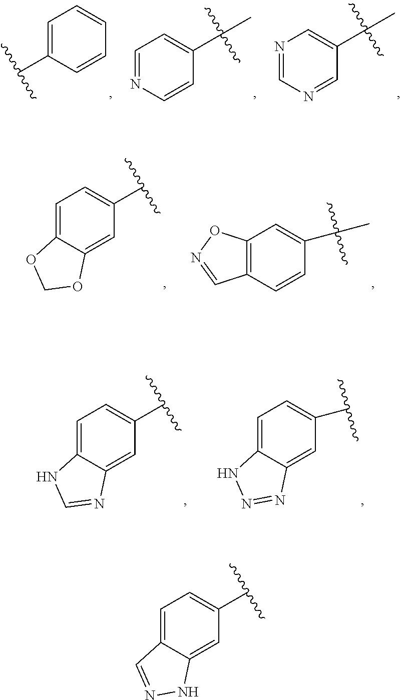
are optionally substituted with one or more substituents selected from hydrogen, halogen, CN, heterocycloalkenyl, hydroxyl, CF₃, C₁₋₆ alkyl, C₁₋₆ alkoxy, C₃₋₆ cycloalkyl, halogen-substituted C₁₋₆ alkoxy, 3- to 6-membered heterocycloalkyl, hydroxyl-substituted C₁₋₆ alkoxy-C₃₋₆ cycloalkyl, hydroxyl-substituted C₁₋₆ alkyl, hydroxyl-substituted C₁₋₆ alkoxy, —NH₂, —N(C₁₋₆ alkyl)₂, —NH(C₁₋₆ alkyl);
preferably, R³, R⁷ are selected from hydrogen, 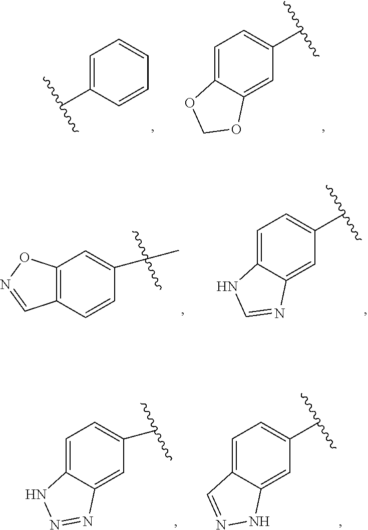
the 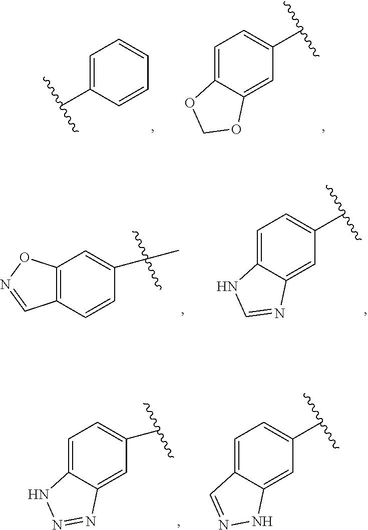
are optionally substituted with one or more substituents selected from hydrogen, halogen, CN, hydroxyl, CF₃, C₁₋₆ alkyl, C₁₋₆ alkoxy, C₃₋₆ cycloalkyl, halogen-substituted C₁₋₆ alkoxy, 3- to 6-membered heterocycloalkyl, hydroxyl-substituted C₁₋₆ alkoxy-C₃₋₆ cycloalkyl, hydroxyl-substituted C₁₋₆ alkyl, hydroxyl-substituted C₁₋₆ alkoxy, —NH₂, —N(C₁₋₆ alkyl)₂, —NH(C₁₋₆ alkyl);
preferably, R³, R⁷ are selected from hydrogen, 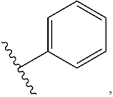 [SMILES: *c1ccccc1]
the 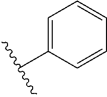 [SMILES: *c1ccccc1]
is optionally substituted with one or more substituents selected from hydrogen, halogen, heterocycloalkenyl, hydroxyl.

22. The compound or tautomer, stereoisomer, solvate, metabolite, isotopically-labeled compound, pharmaceutically acceptable salt or co-crystal thereof according to claim 17, wherein,
R⁵ is each independently selected at each occurrence from hydrogen, halogen, CN, hydroxyl, C₁₋₆ alkyl, C₁₋₆ alkoxy, —O, C₂₋₆ alkenyl, C₆₋₁₀ aryl or C₆₋₁₀ heteroaryl, wherein, the alkyl, alkoxy, alkenyl, aryl or heteroaryl is optionally substituted with one or more substituents selected from hydrogen, halogen, CN, NO₂, CF₃, CHF₂, hydroxyl, C₁₋₆ alkyl, C₁₋₆ alkoxy, —C(═O)—C₁₋₆ alkoxy, C₂₋₆ alkenyl, —C(═O)—NH₂, the heteroaryl, heterocycloalkyl contain 1 to 4 heteroatoms optionally selected from N, O or S;
preferably, R⁵ is each independently selected at each occurrence from hydrogen, CN, C₁₋₆ alkoxy, ═O, C₆₋₁₀ aryl or C₆₋₁₀ heteroaryl, wherein, the alkoxy, aryl or heteroaryl is optionally substituted with one or more substituents selected from hydrogen, halogen, CN, NO₂, CF₃, CHF₂, hydroxyl, C₁₋₆ alkyl, —C(═O)—C₁₋₆ alkoxy, —C(═O)—NH₂, the heteroaryl, heterocycloalkyl contain 1 to 4 heteroatoms optionally selected from N, O or S;
alternatively, R⁵, R⁶ and the atom to which both of them directly connect collectively form cycloalkyl, heteroalicyclic, aryl or heteroaryl, wherein, the cycloalkyl, heteroalicyclic, aryl, heteroaryl are optionally substituted with one or more substituents selected from hydrogen, halogen, CN, hydroxyl, C₁₋₆ alkyl, C₁₋₆ alkoxy, C₂₋₆ alkenyl, C₂₋₆ alkynyl;
preferably, R⁵ is each independently selected from CN, C₁₋₆ alkoxy, ═O, 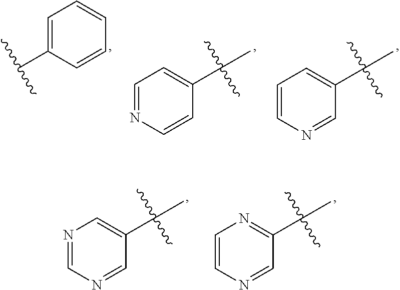
wherein, the alkoxy, 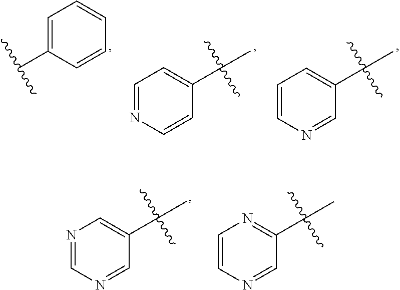
are optionally substituted with one or more substituents selected from hydrogen, halogen, CN, NO₂, CF₃, CHF₂, hydroxyl, C₁₋₆ alkyl, —C(═O)—NH₂, —C(O)OCH₃;
alternatively, R⁵, R⁶ and the atom to which both of them directly connect collectively form 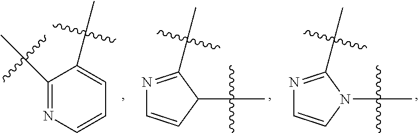
wherein, the 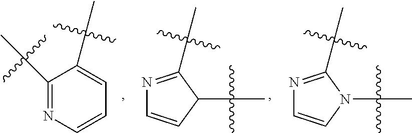
are optionally substituted with one or more groups selected from hydrogen, halogen, CN, hydroxyl, C₁₋₆ alkyl, C₁₋₆ alkoxy;
preferably, R⁵ is selected from C₁₋₆ alkoxy, 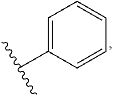
wherein, the 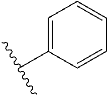 [SMILES: *c1ccccc1]
is optionally substituted with one or more substituents selected from hydrogen, halogen, CN, NO₂;
alternatively, R⁵, R⁶ and the atom to which both of them directly connect collectively form

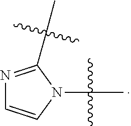 [SMILES: *c1nccn1*]

23. The compound or tautomer, stereoisomer, solvate, metabolite, isotopically-labeled compound, pharmaceutically acceptable salt or co-crystal thereof according to claim 17, wherein,
X, Y are each independently selected from C, N;
Z is selected from a bond, —CH₂—, —C(═O) or —S(═O)₂—;
preferably, Z is selected from a bond, —CH₂— or —C(═O);
preferably, Z is selected from a bond;
preferably R^{a}, R^{b} are each independently selected at each occurrence from hydrogen, hydroxyl, C₁₋₆ alkyl, C₁₋₆ alkoxy, C₂₋₆ alkenyl, C₂₋₆ alkynyl, C₃₋₆ cycloalkyl, halogen-substituted C₁₋₆ alkyl, the heteroaryl, heterocycloalkyl contain 1 to 4 heteroatoms optionally selected from N, O or S;
preferably, R^{a}, R^{b} are each independently selected at each occurrence from hydrogen, C₁₋₆ alkyl;
preferably, R^{a}, R^{b} are each independently selected at each occurrence from hydrogen.

24. (canceled)

25. The following compound or tautomer, stereoisomer, solvate, metabolite, isotopically-labeled compound, pharmaceutically acceptable salt or co-crystal thereof:

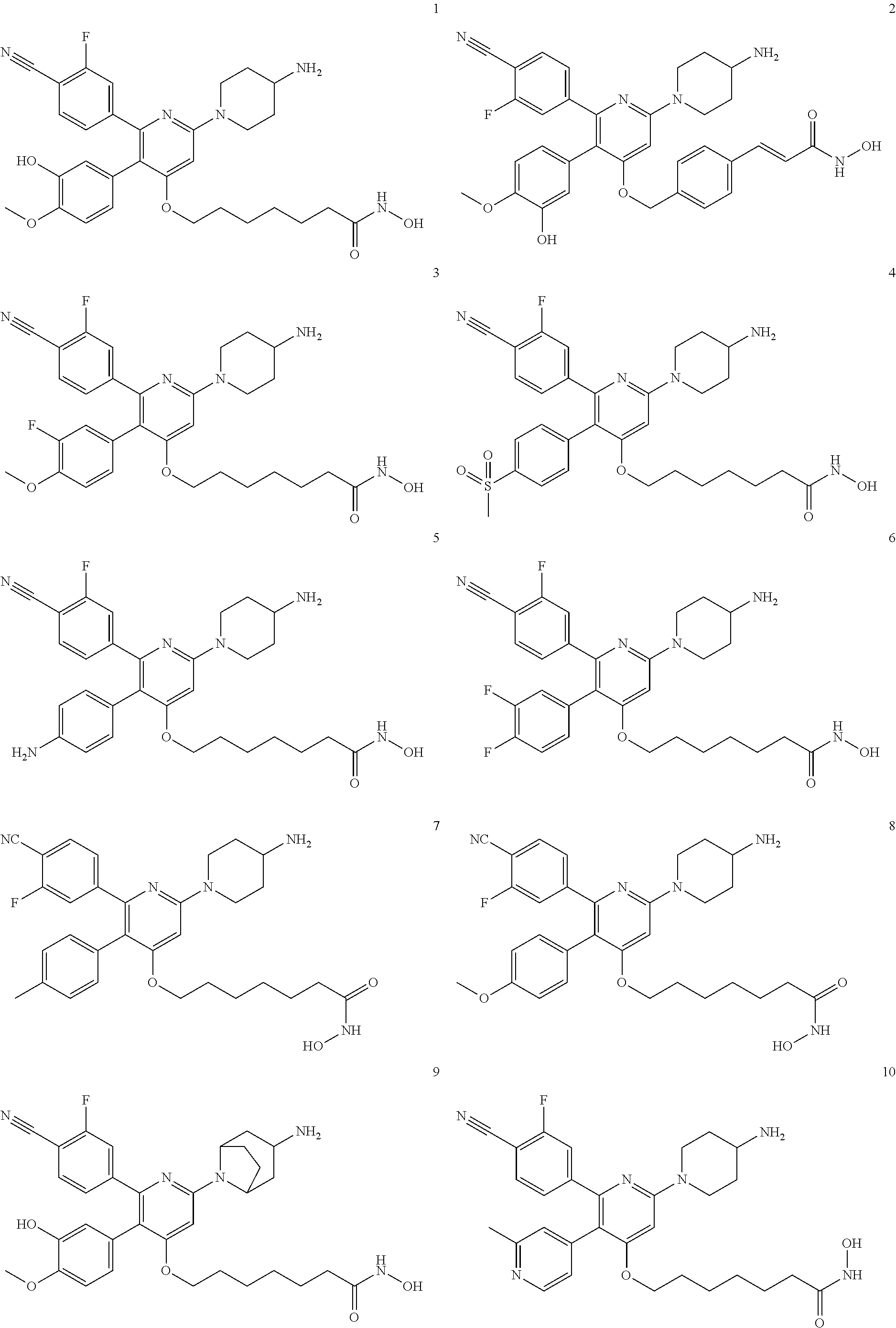

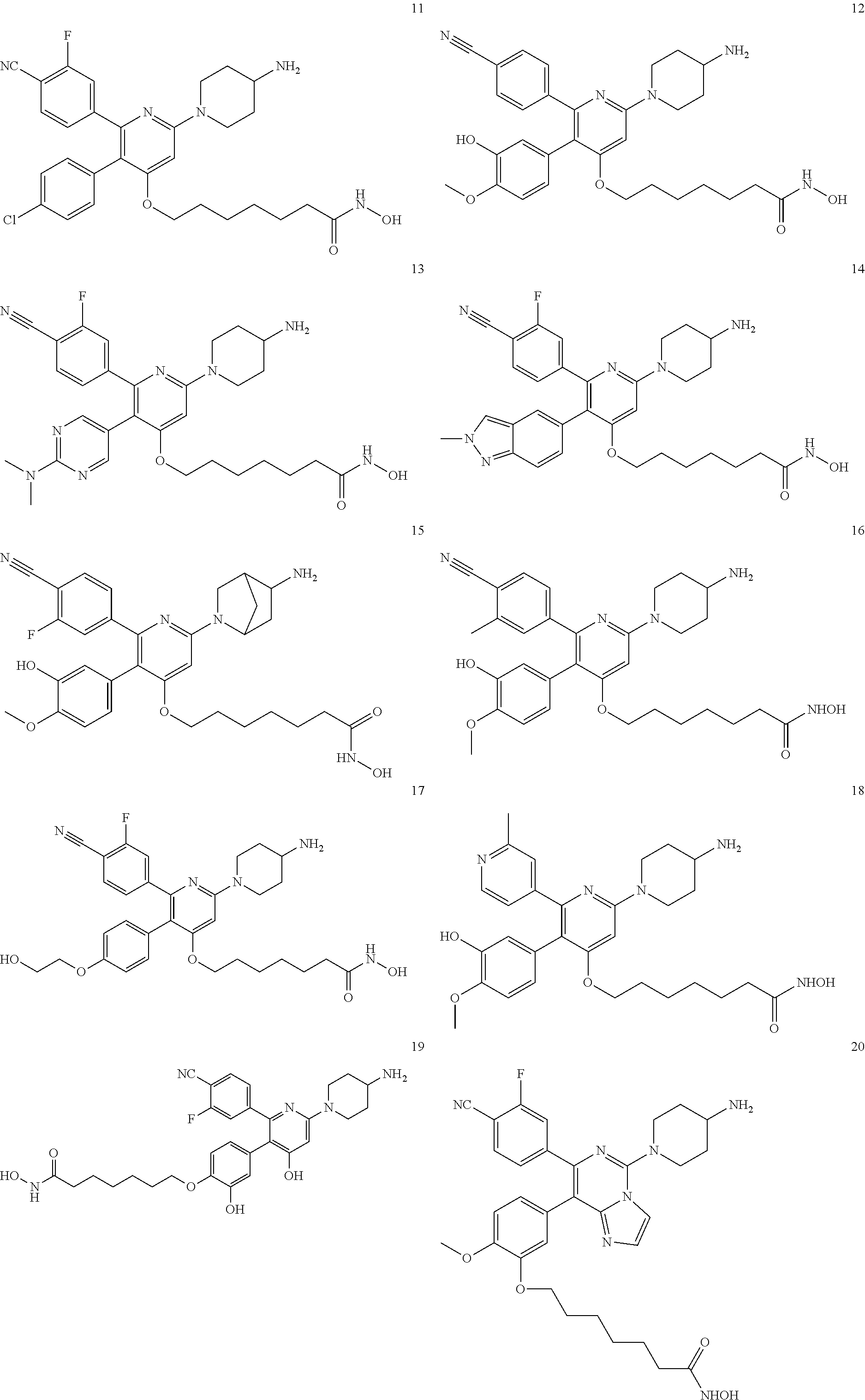

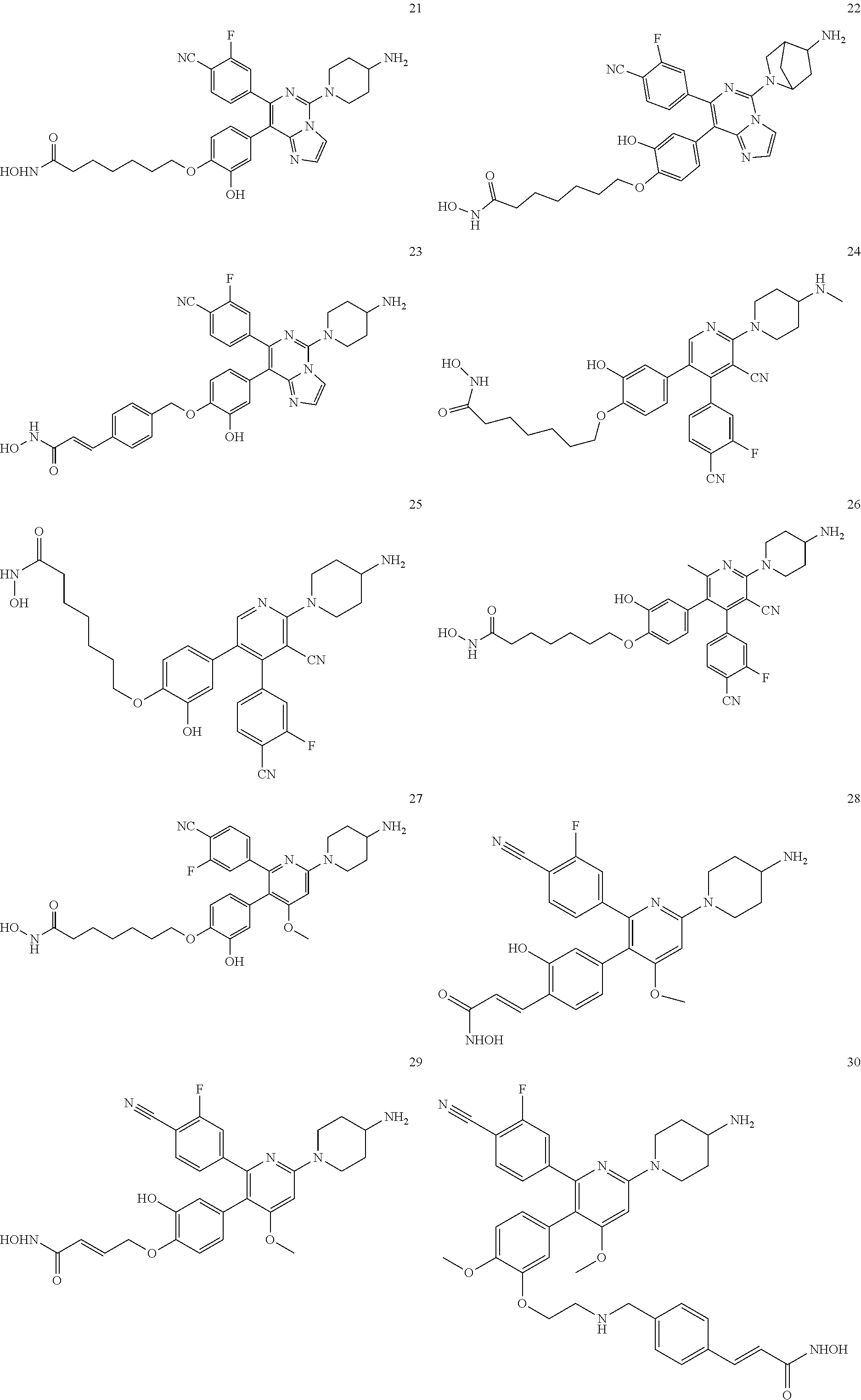

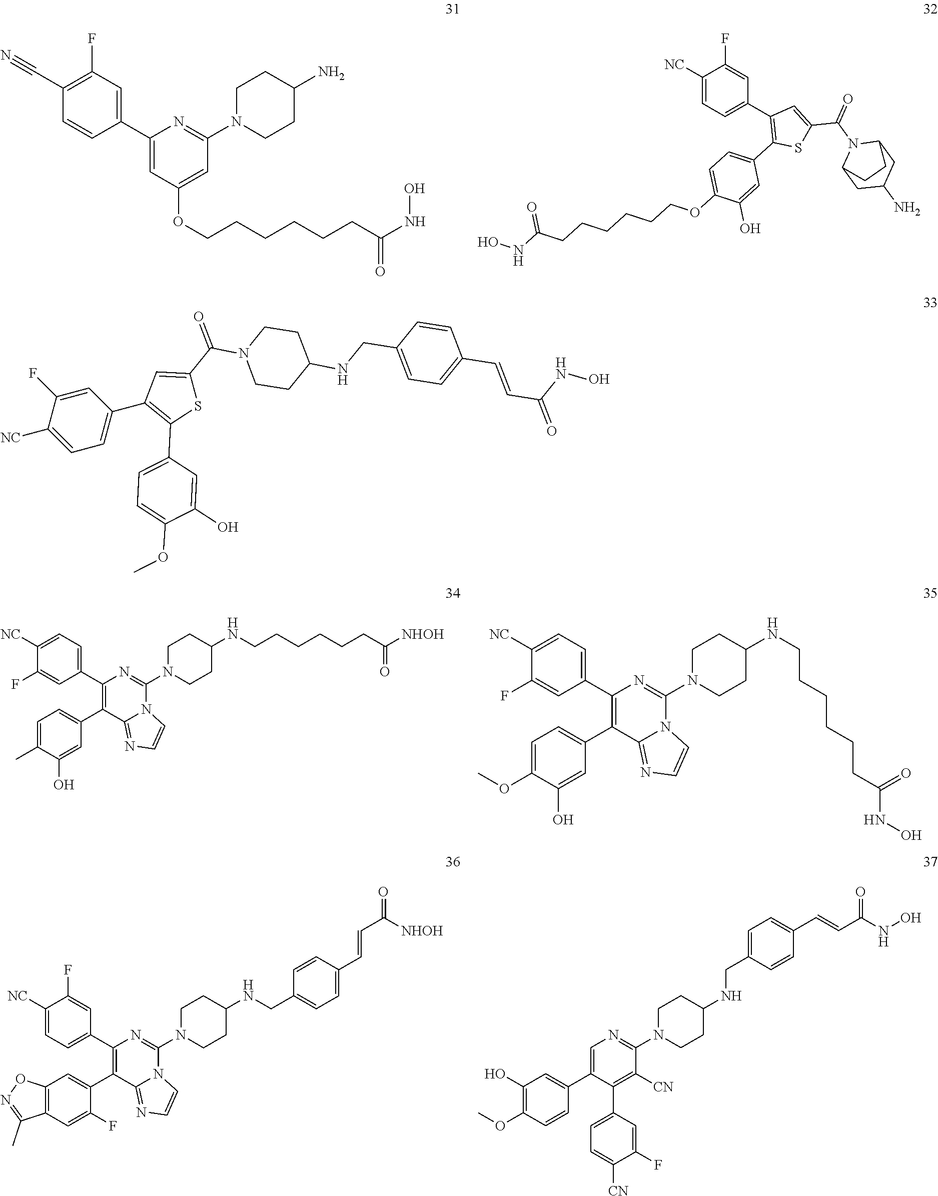

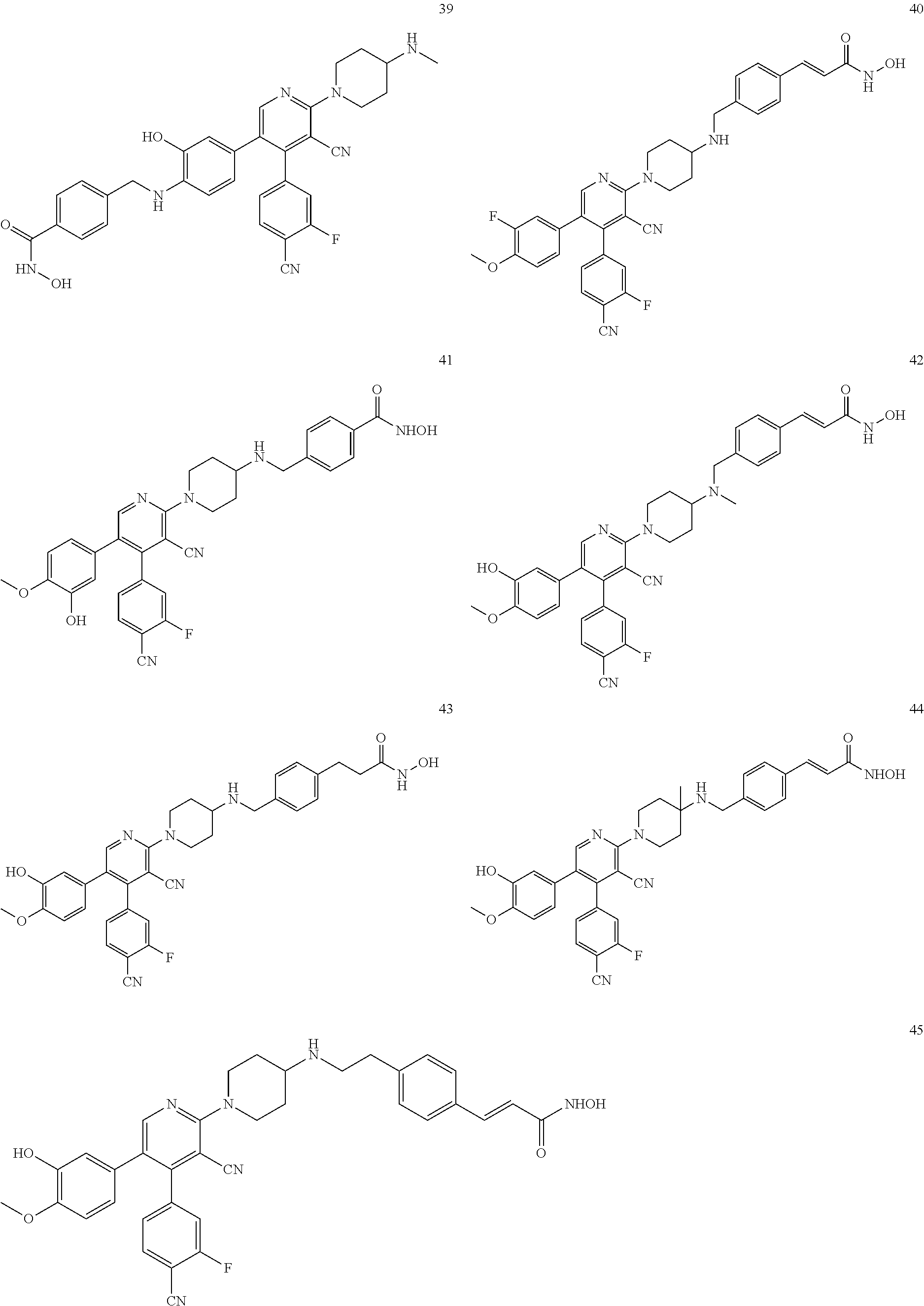

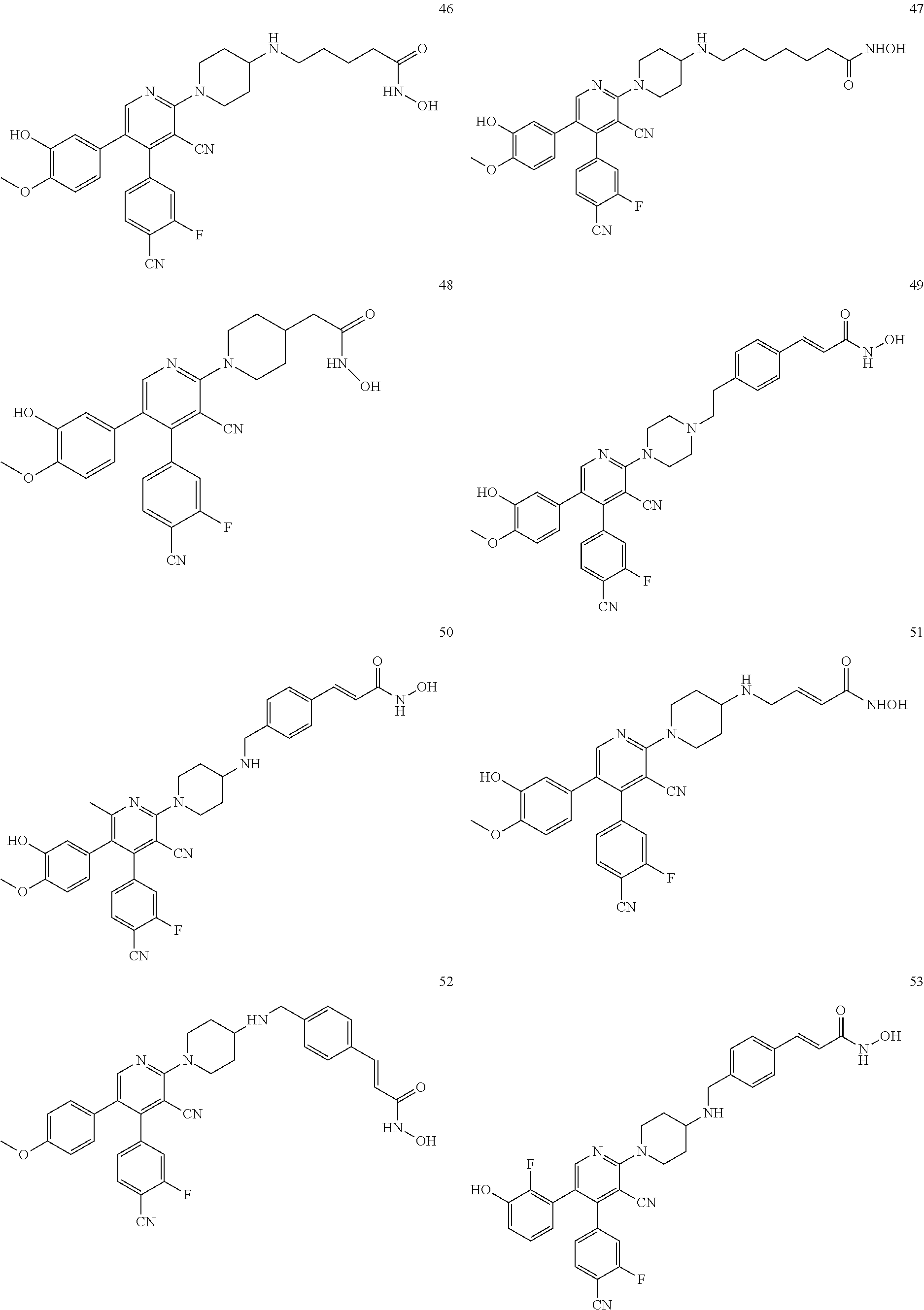

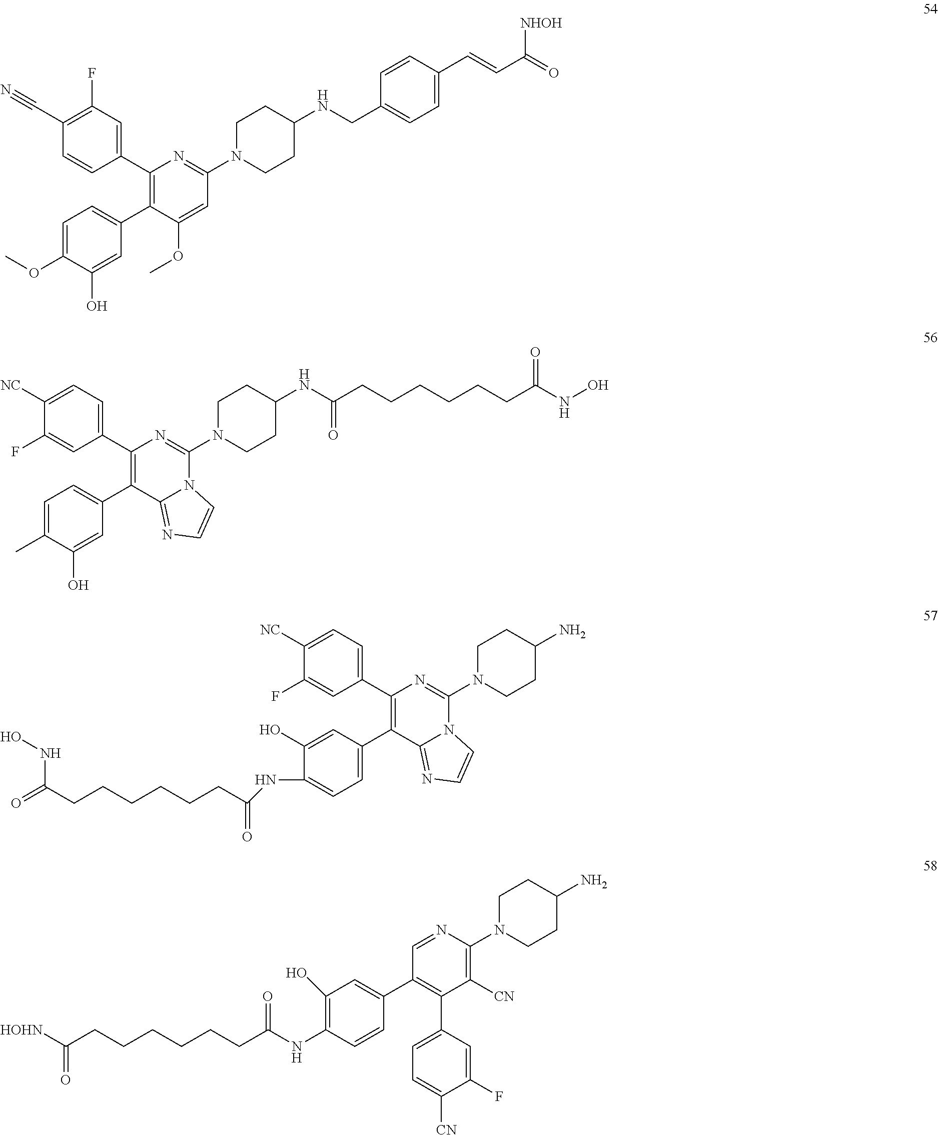

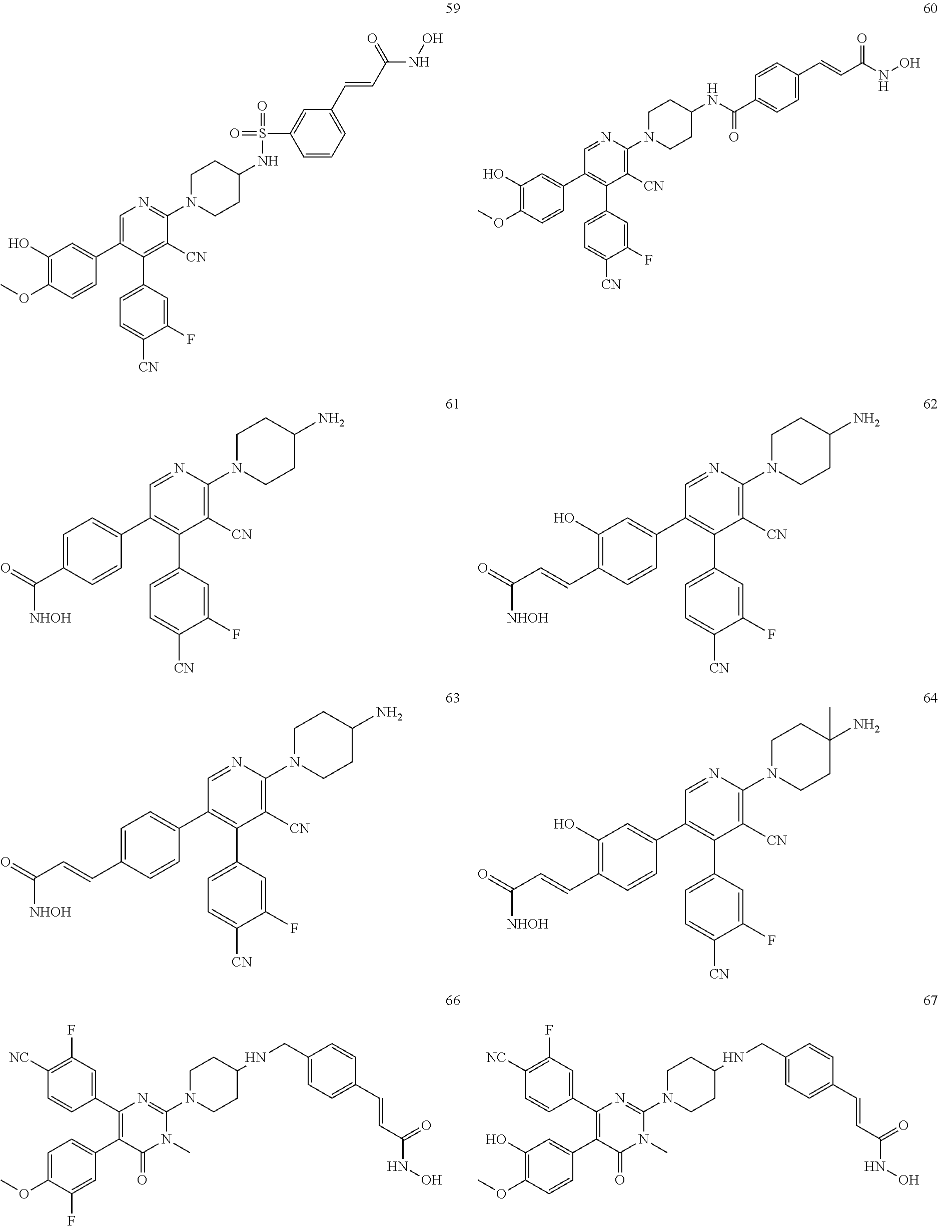

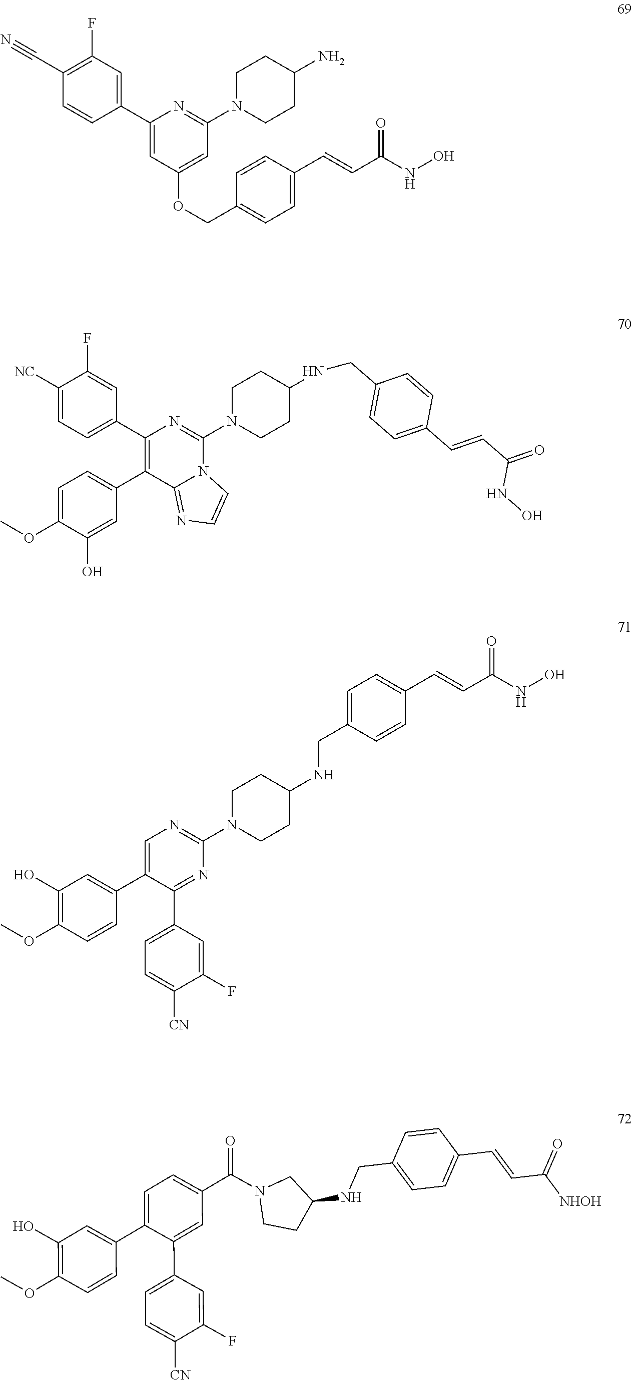

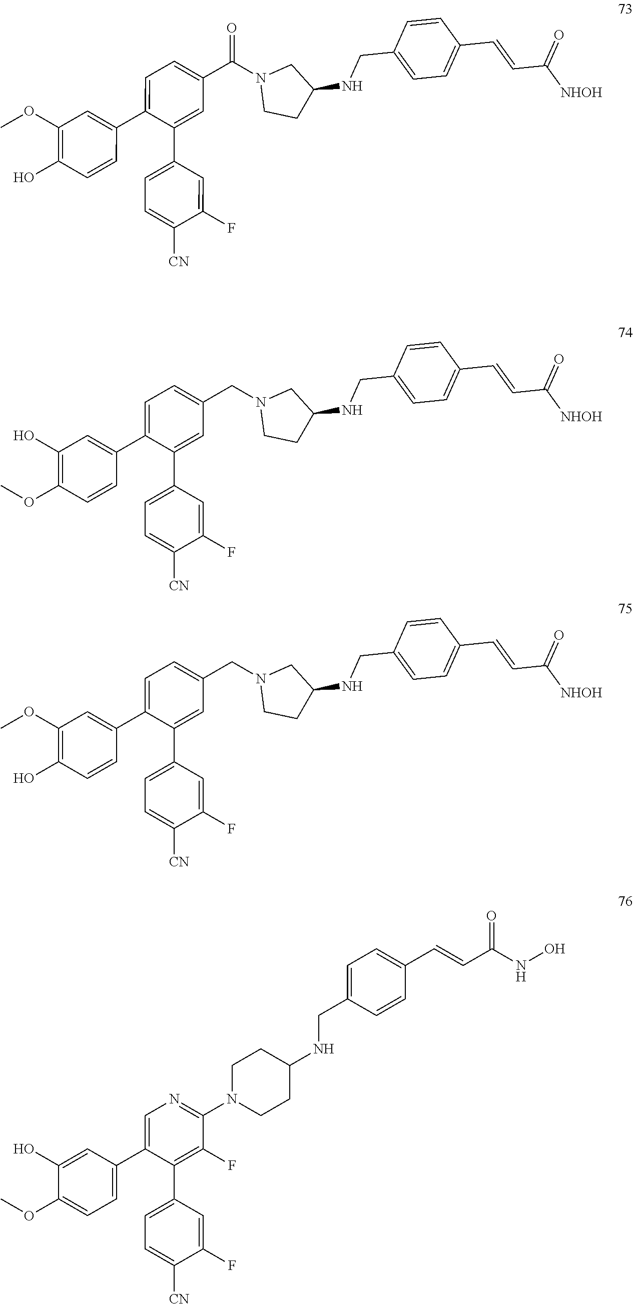

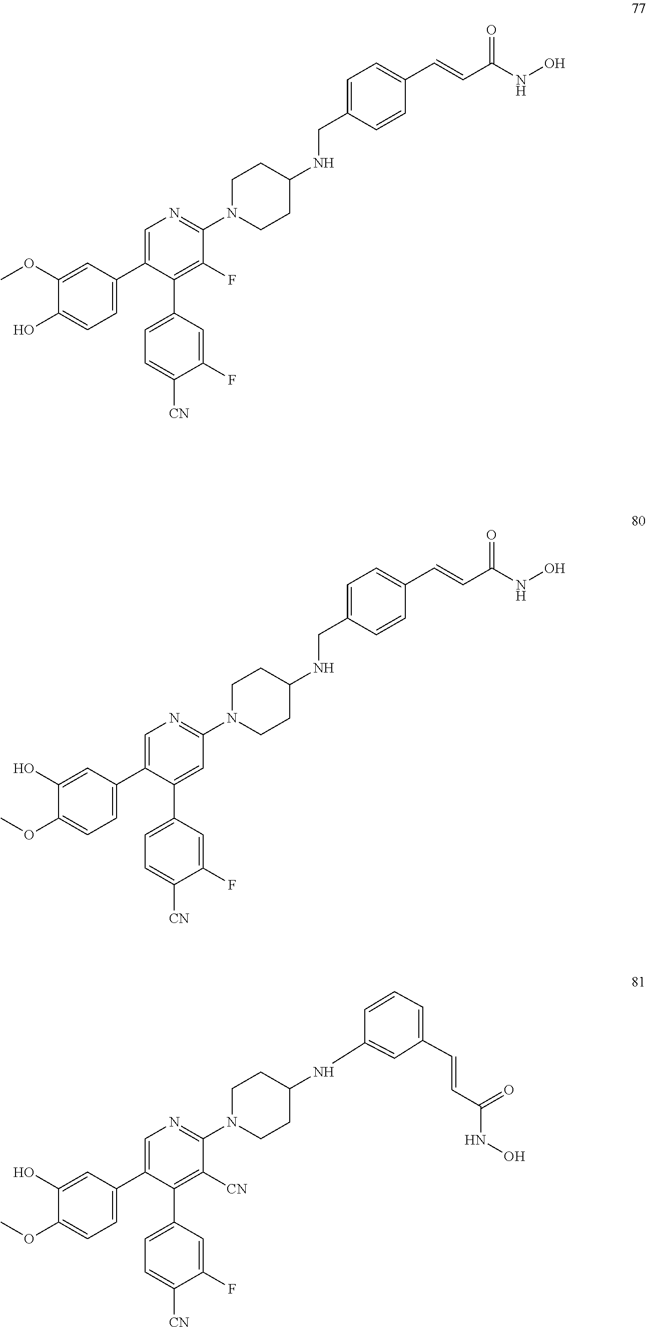 [SMILES: COc1cc(-c2cnc(N3CCC(NCc4ccc(/C=C/C(=O)NO)cc4)CC3)c(F)c2-c2ccc(C#N)c(F)c2)ccc1O ; COc1ccc(-c2cnc(N3CCC(NCc4ccc(/C=C/C(=O)NO)cc4)CC3)cc2-c2ccc(C#N)c(F)c2)cc1O ; COc1ccc(-c2cnc(N3CCC(Nc4cccc(/C=C/C(=O)NO)c4)CC3)c(C#N)c2-c2ccc(C#N)c(F)c2)cc1O]

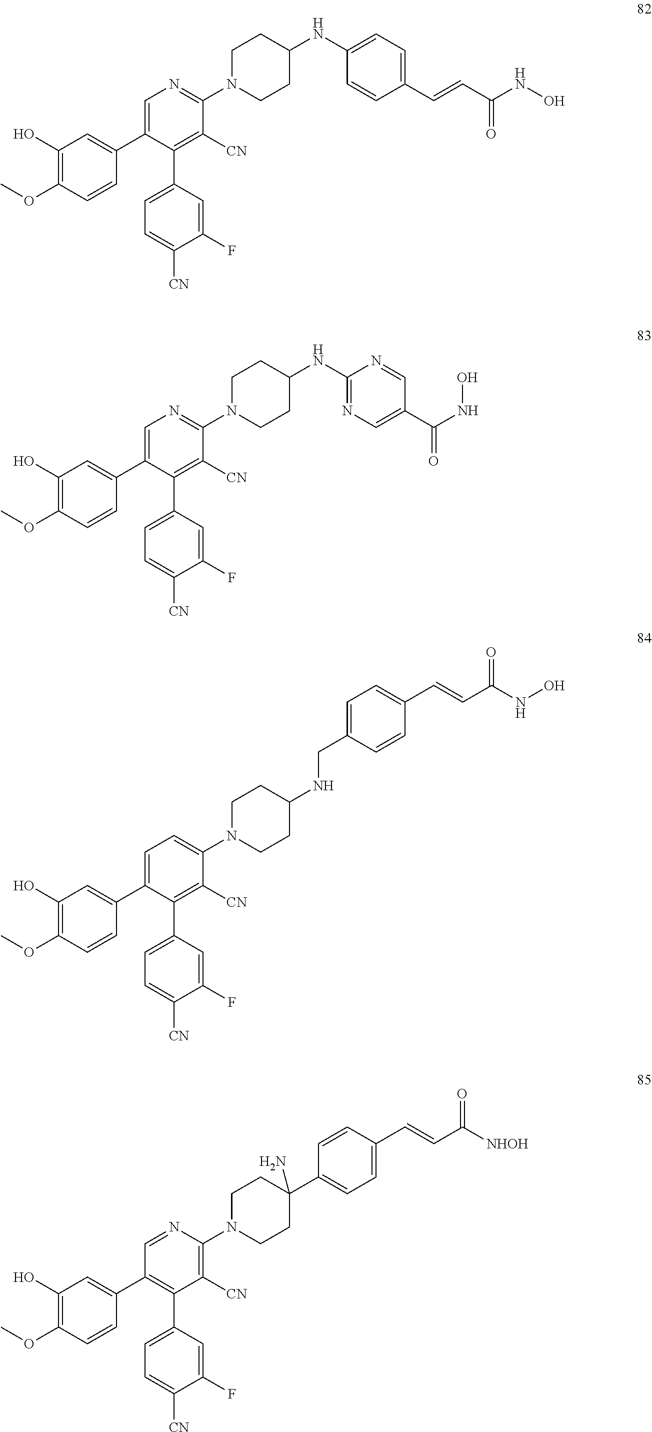

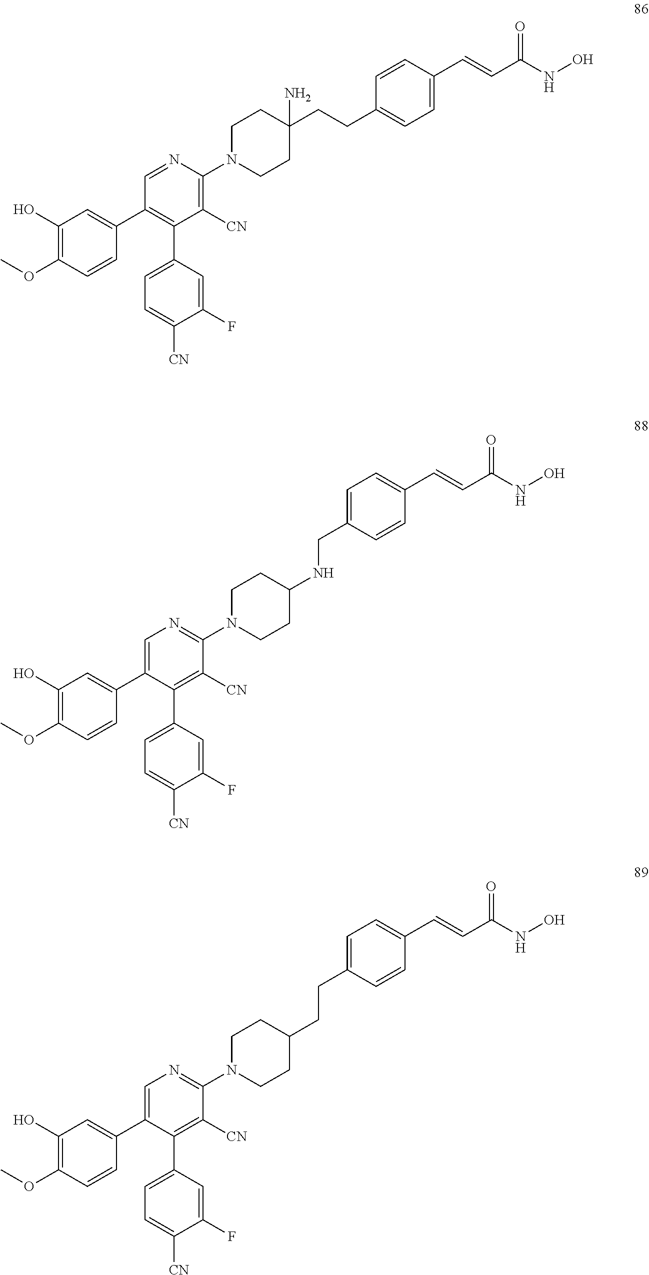 [SMILES: COc1ccc(-c2cnc(N3CCC(N)(CCc4ccc(/C=C/C(=O)NO)cc4)CC3)c(C#N)c2-c2ccc(C#N)c(F)c2)cc1O ; COc1ccc(-c2cnc(N3CCC(NCc4ccc(/C=C/C(=O)NO)cc4)CC3)c(C#N)c2-c2ccc(C#N)c(F)c2)cc1O ; COc1ccc(-c2cnc(N3CCC(CCc4ccc(/C=C/C(=O)NO)cc4)CC3)c(C#N)c2-c2ccc(C#N)c(F)c2)cc1O]

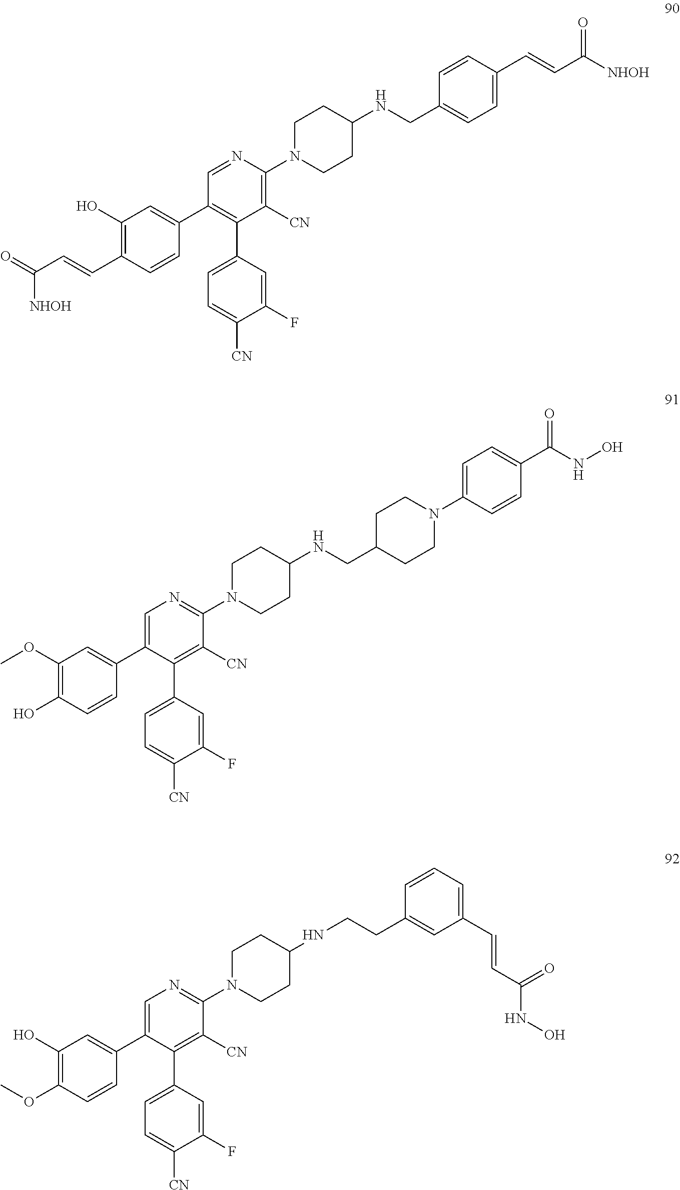

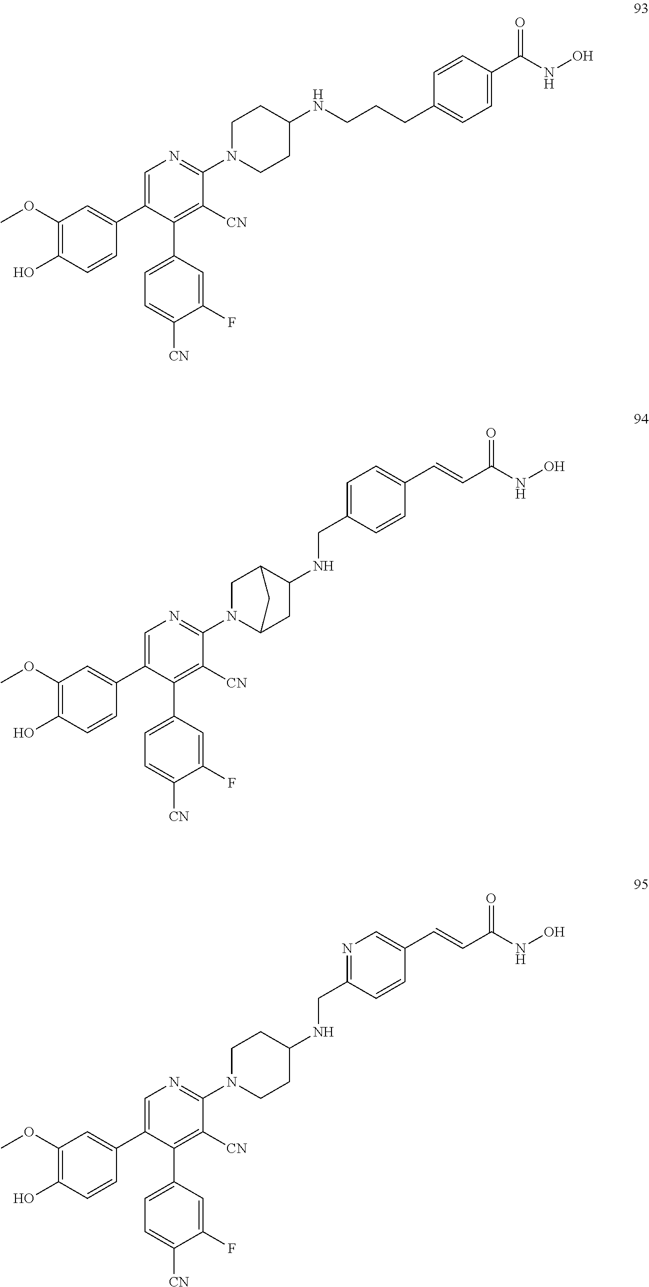 [SMILES: COc1cc(-c2cnc(N3CCC(NCCCc4ccc(C(=O)NO)cc4)CC3)c(C#N)c2-c2ccc(C#N)c(F)c2)ccc1O ; COc1cc(-c2cnc(N3CC4CC3CC4NCc3ccc(/C=C/C(=O)NO)cc3)c(C#N)c2-c2ccc(C#N)c(F)c2)ccc1O ; COc1cc(-c2cnc(N3CCC(NCc4ccc(/C=C/C(=O)NO)cn4)CC3)c(C#N)c2-c2ccc(C#N)c(F)c2)ccc1O]

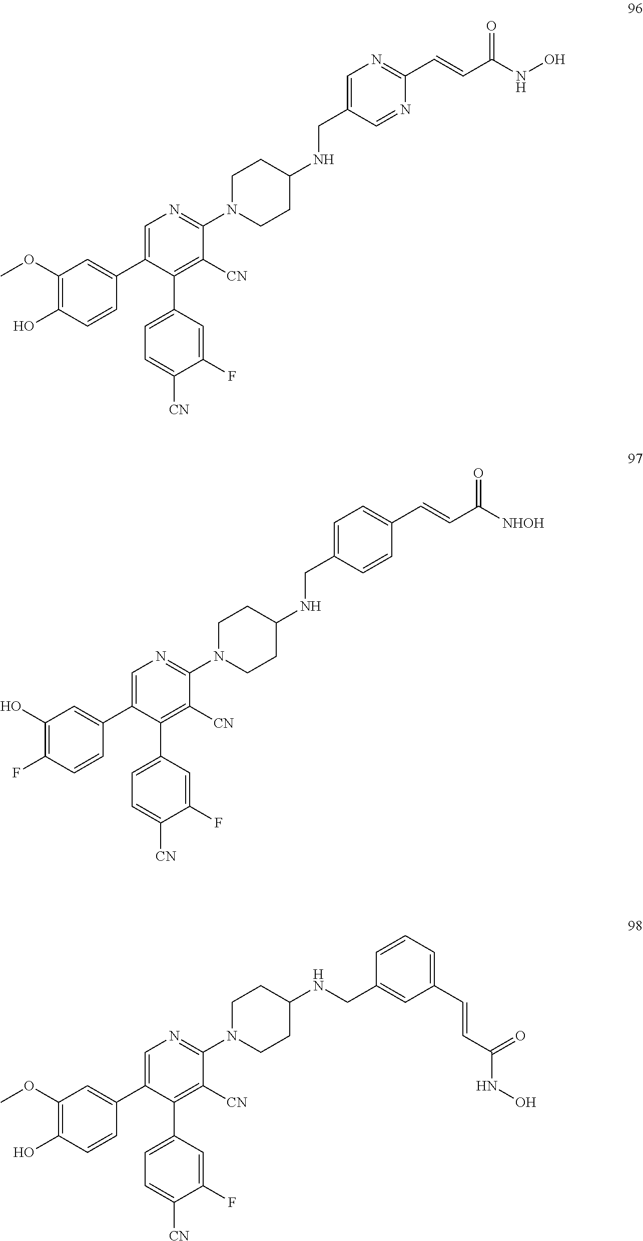

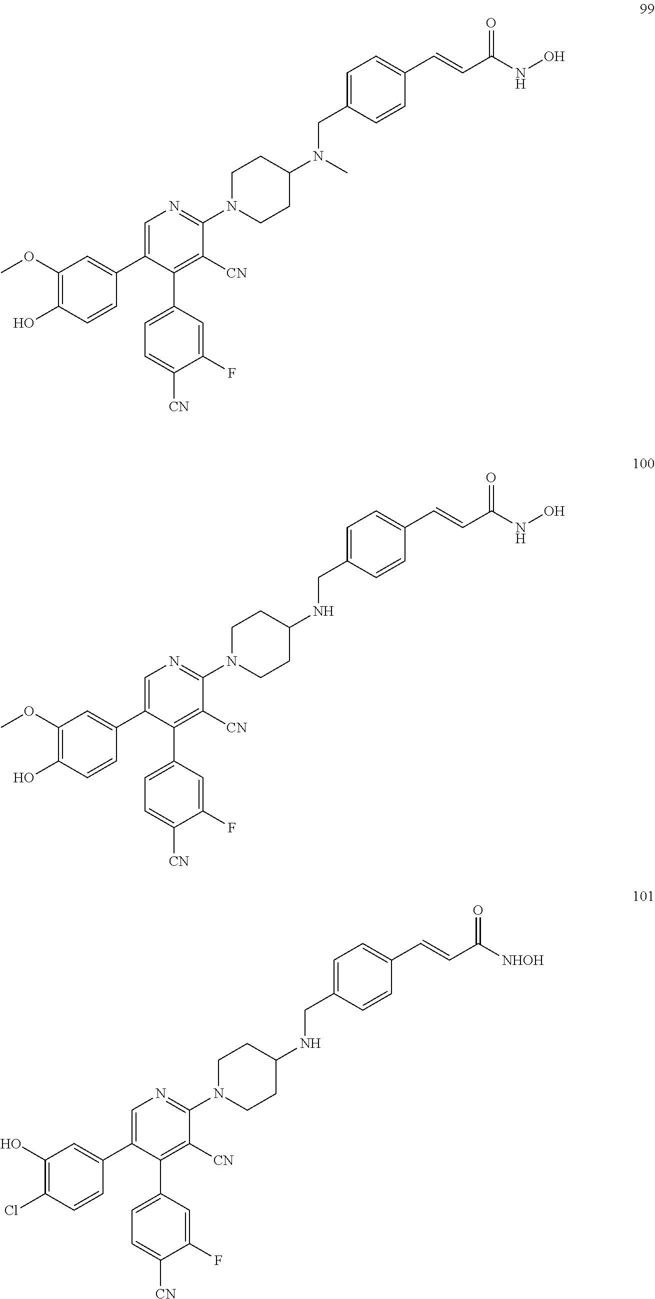

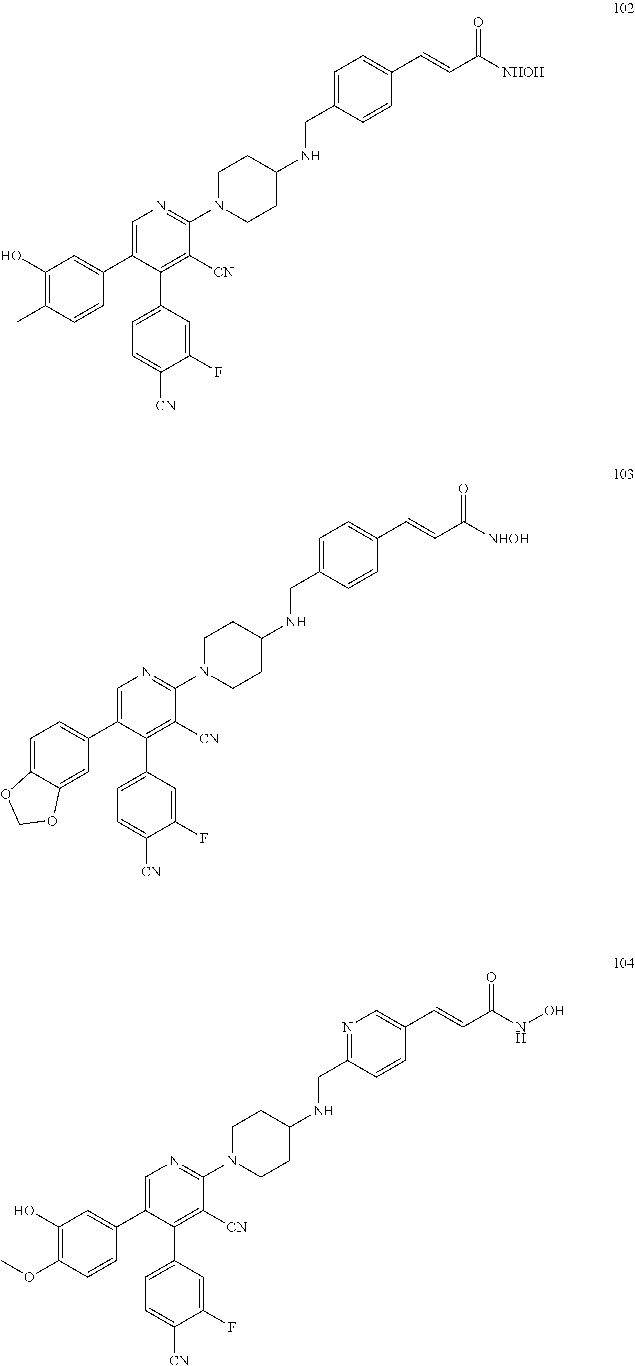

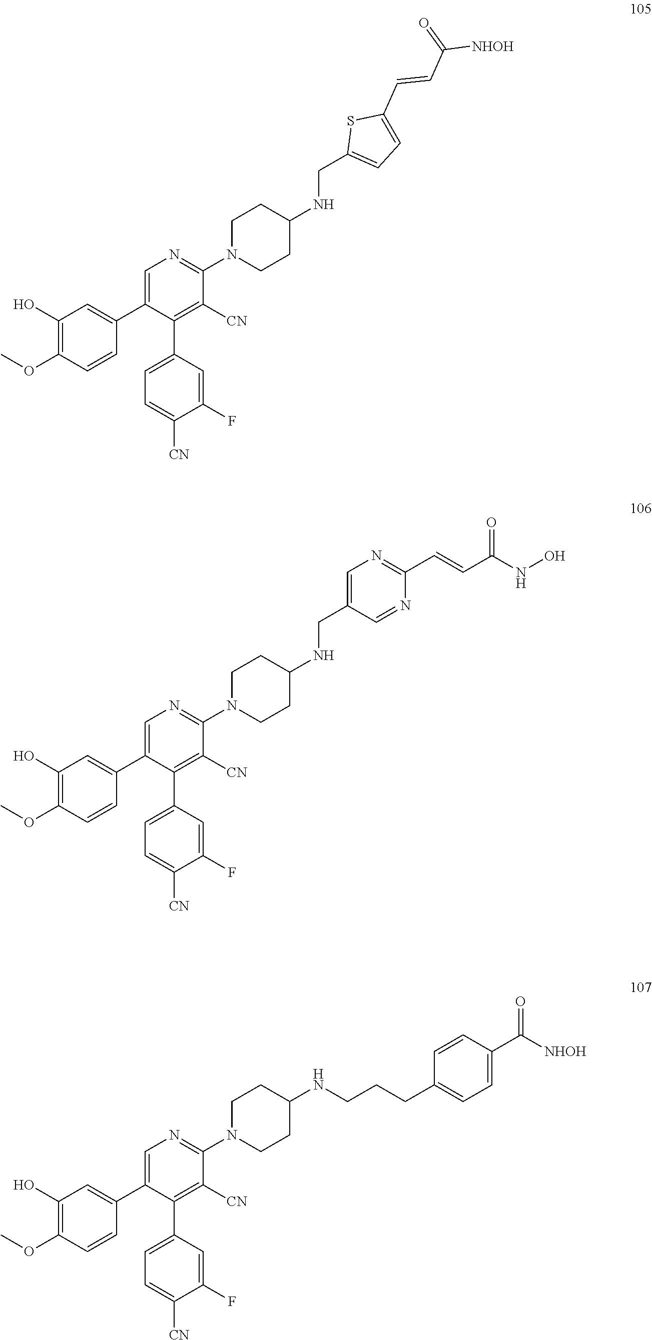

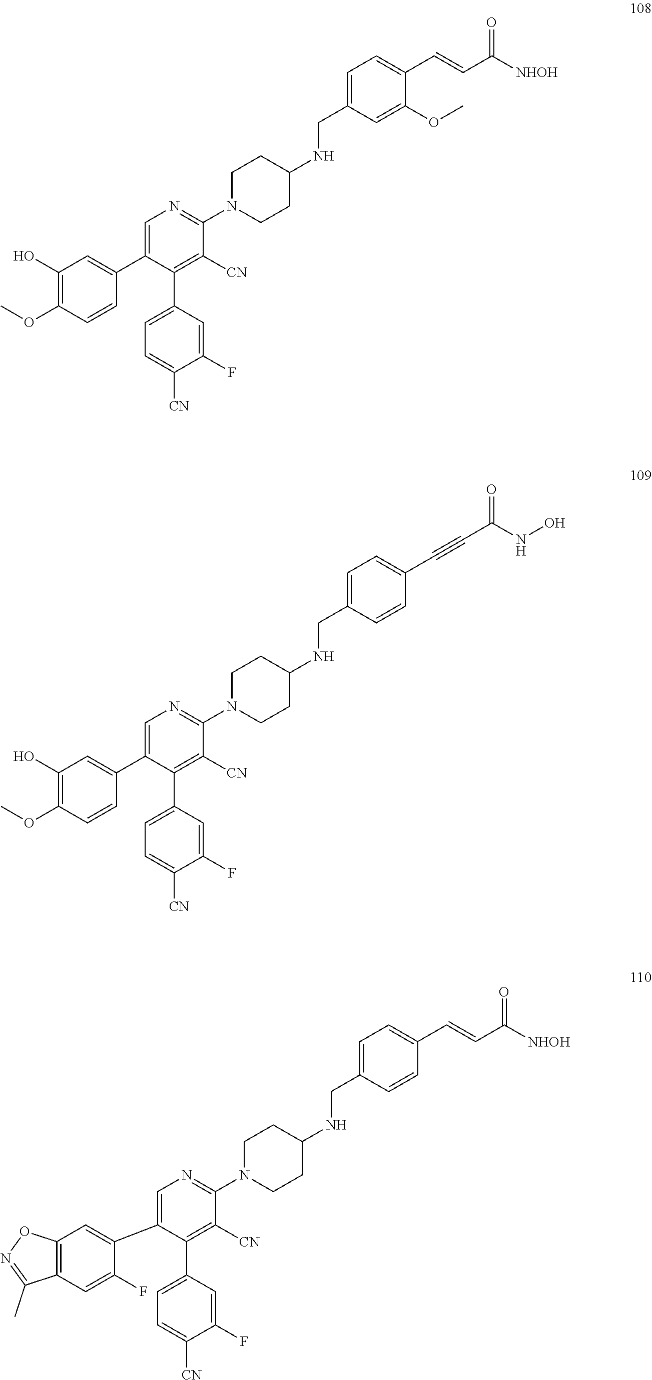

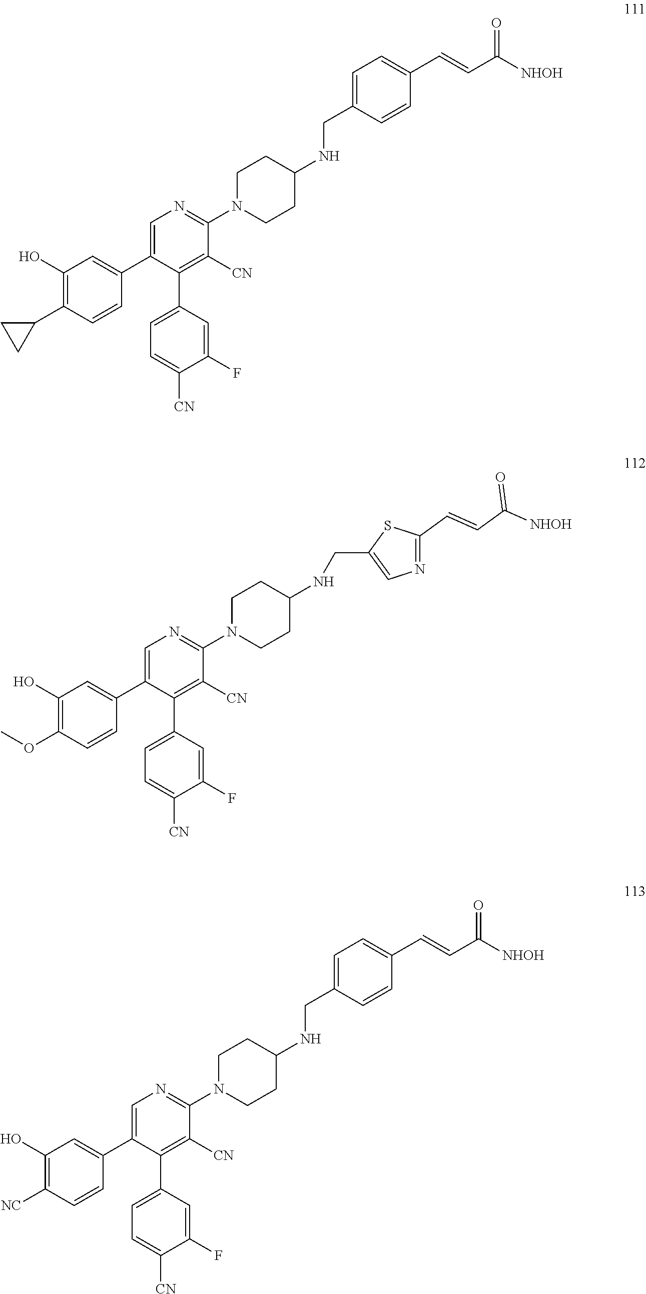

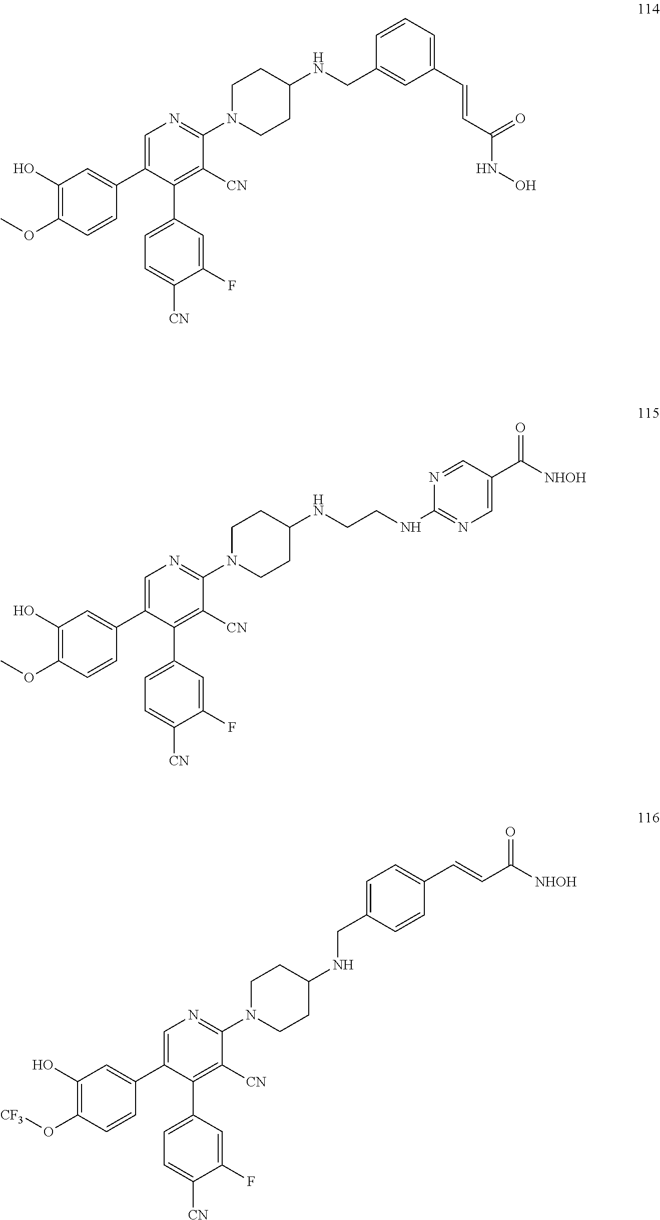

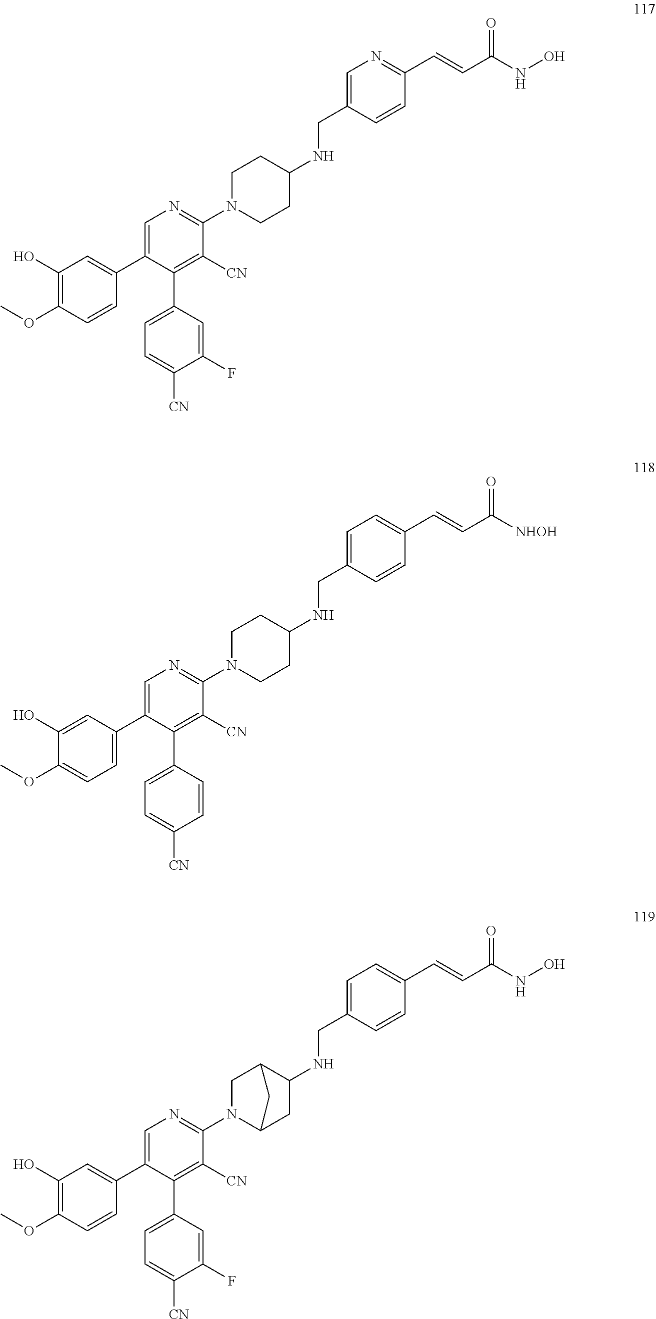

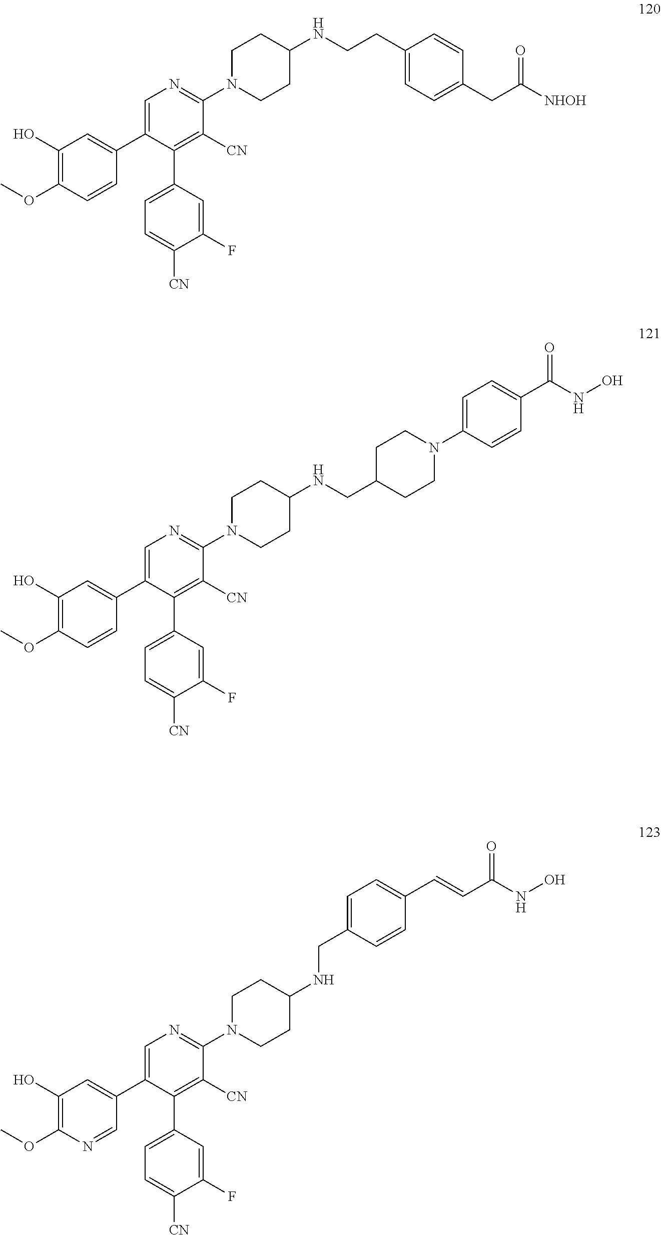

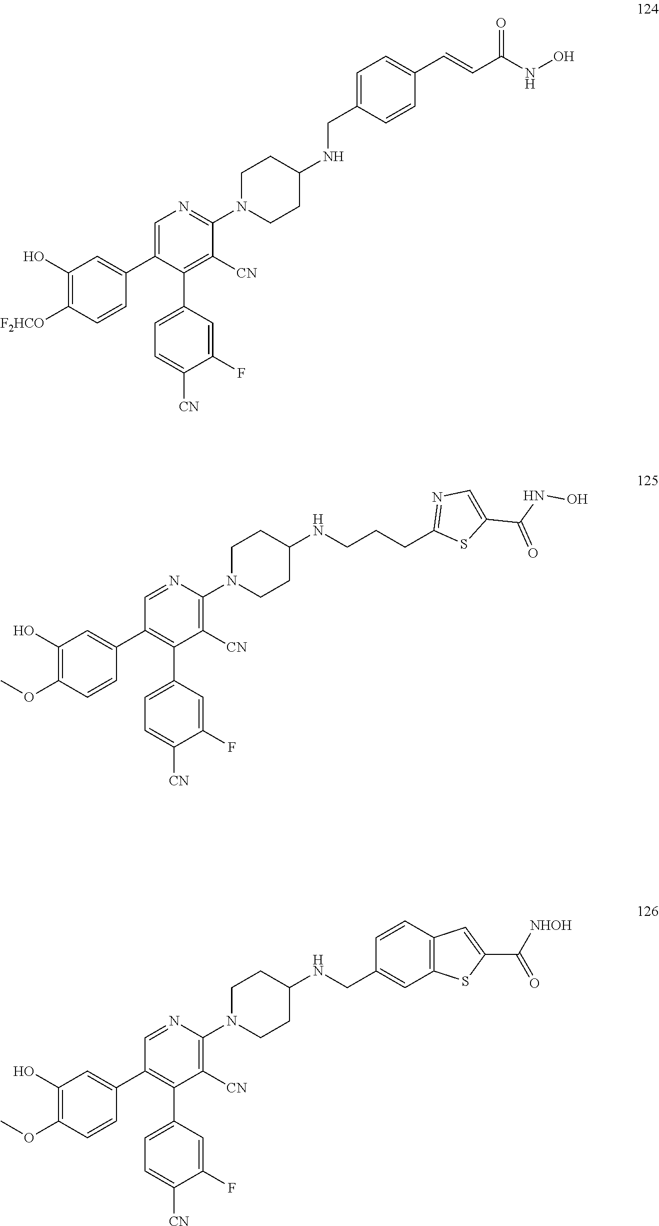

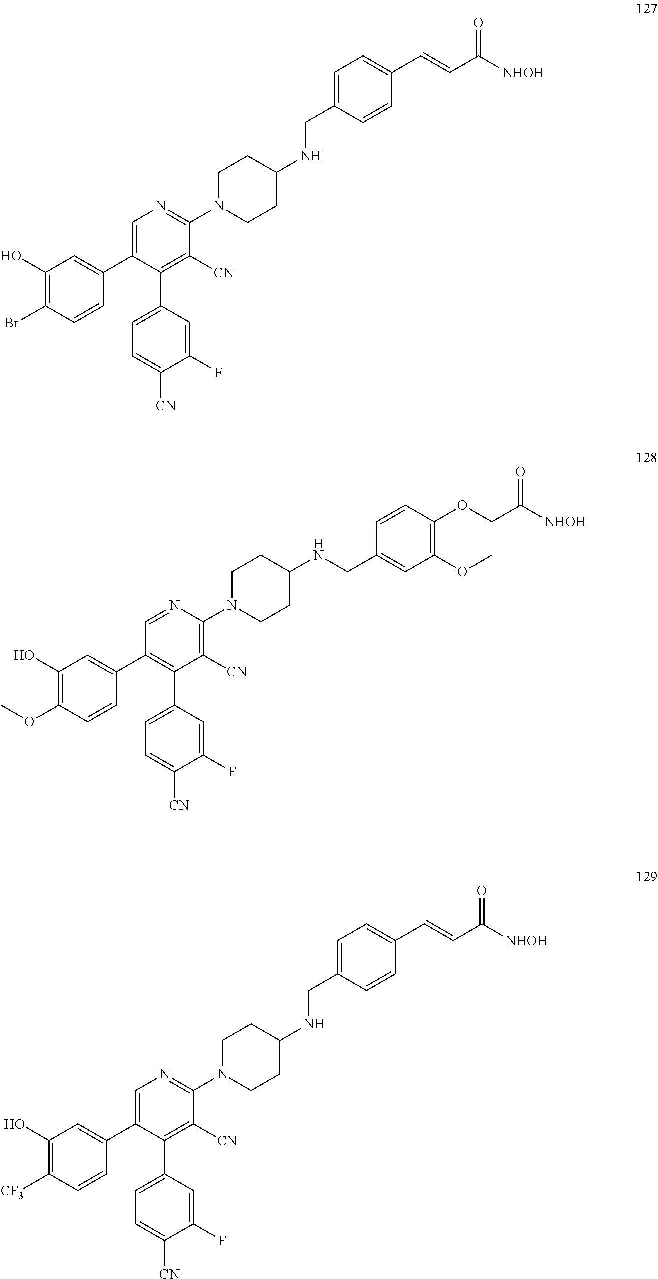

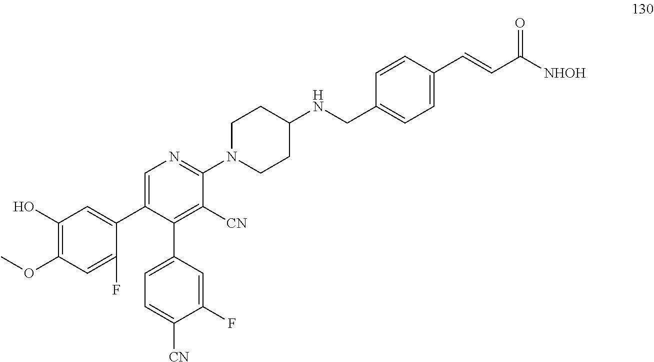

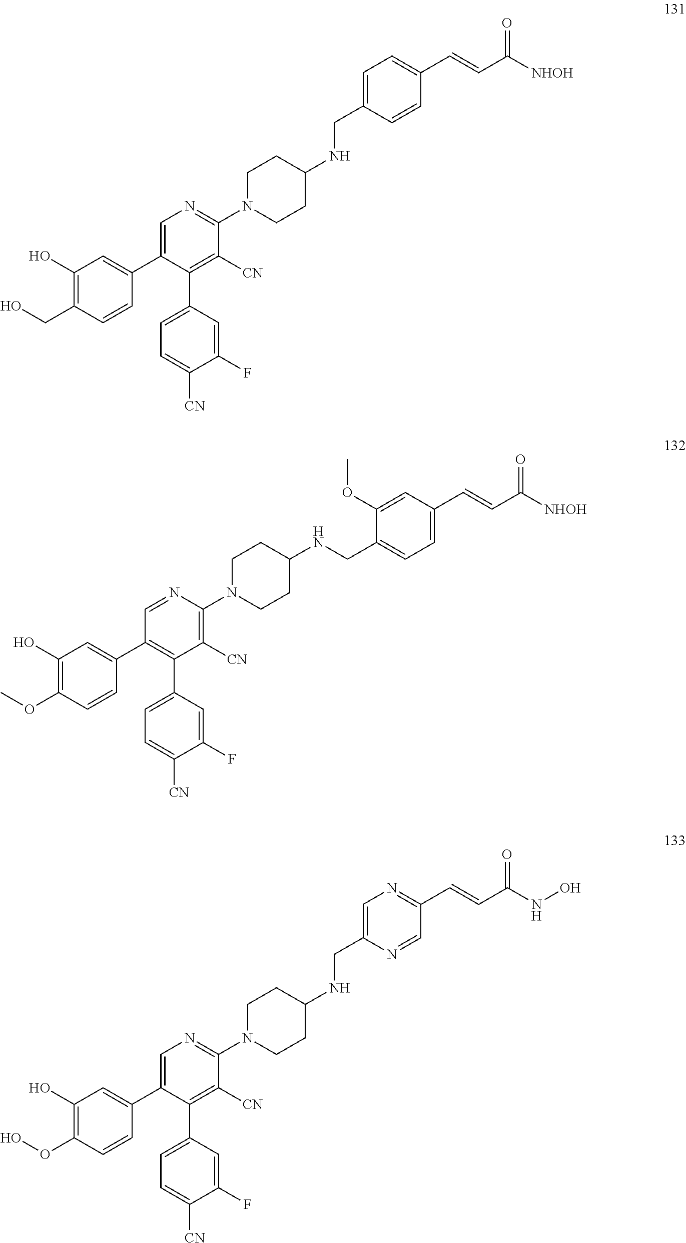

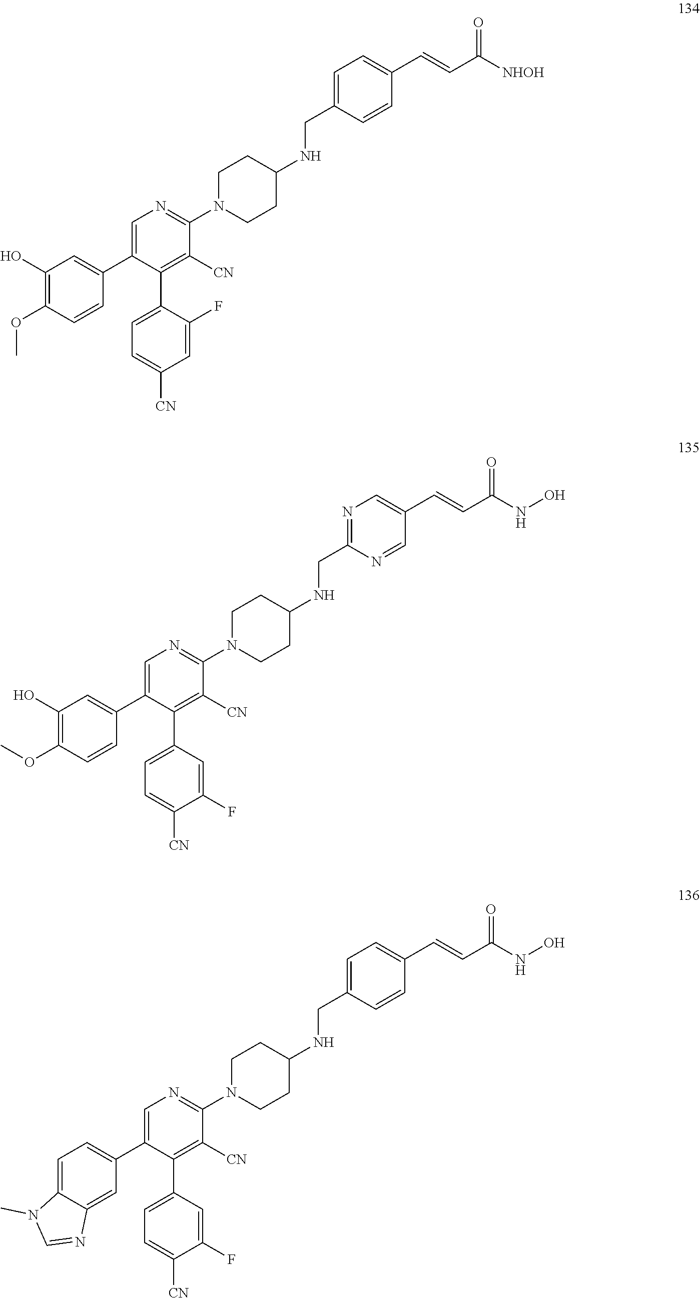

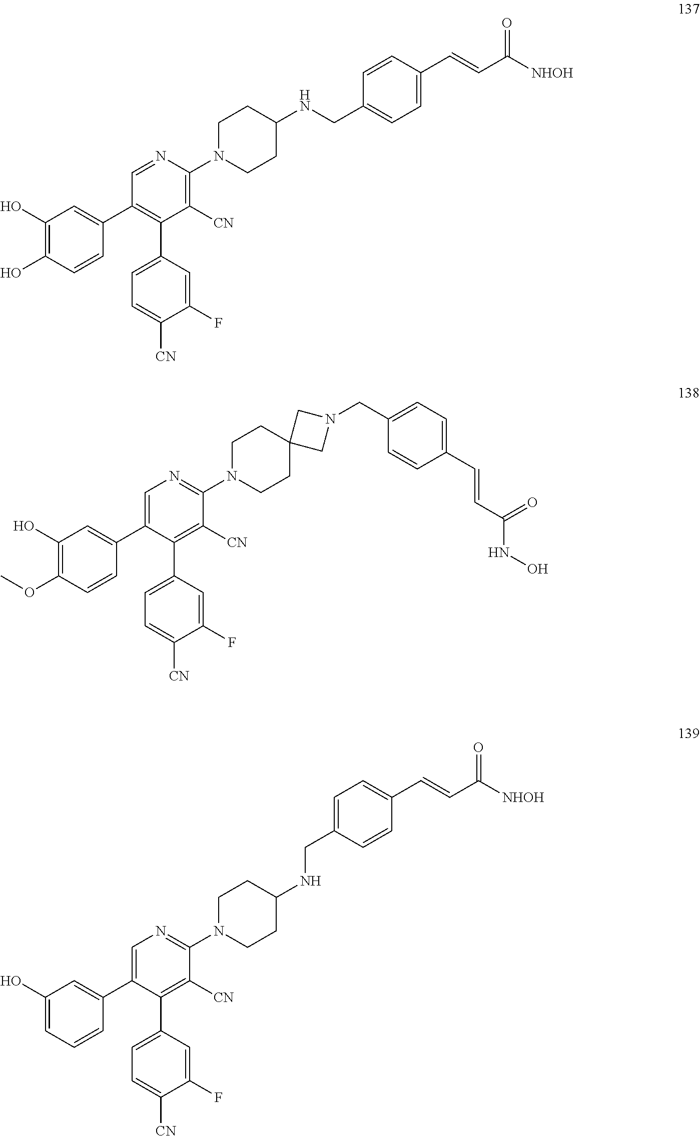

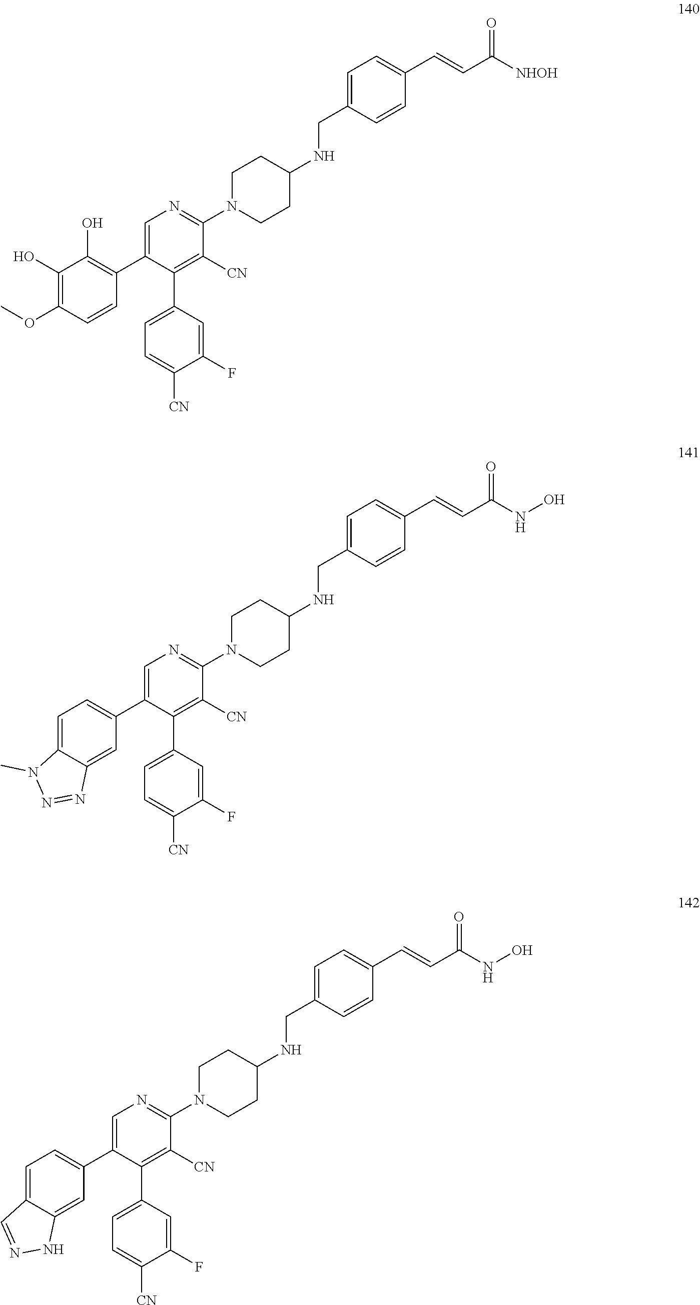

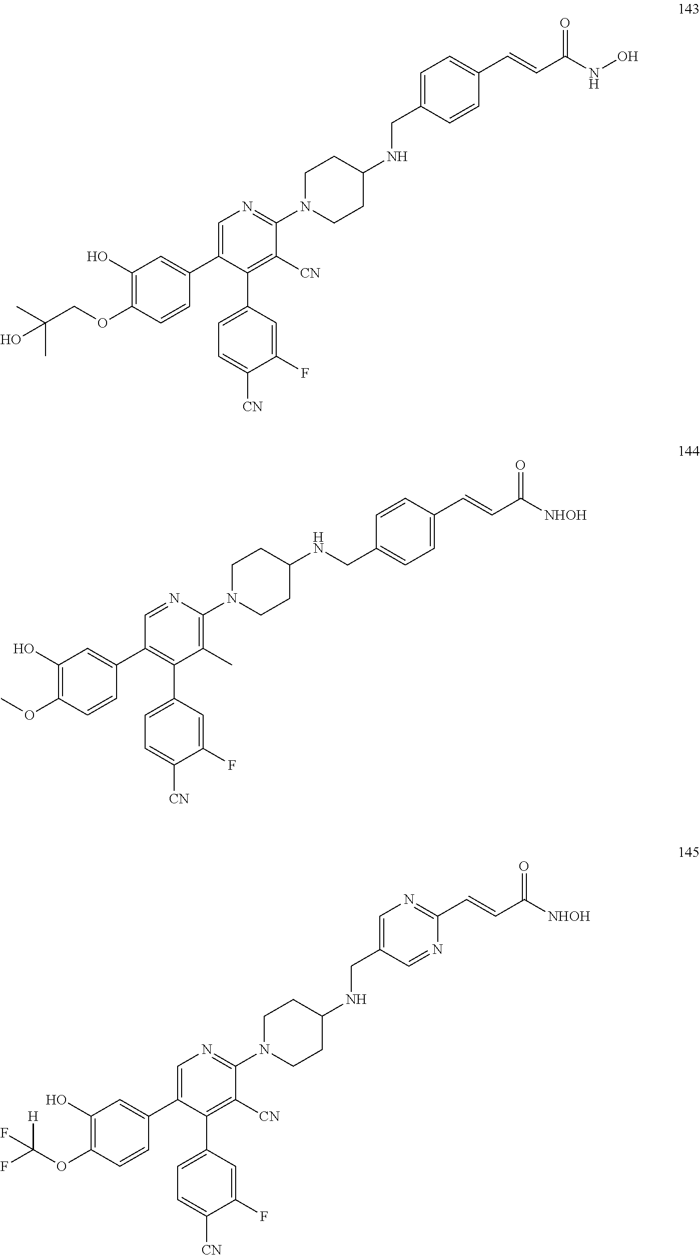

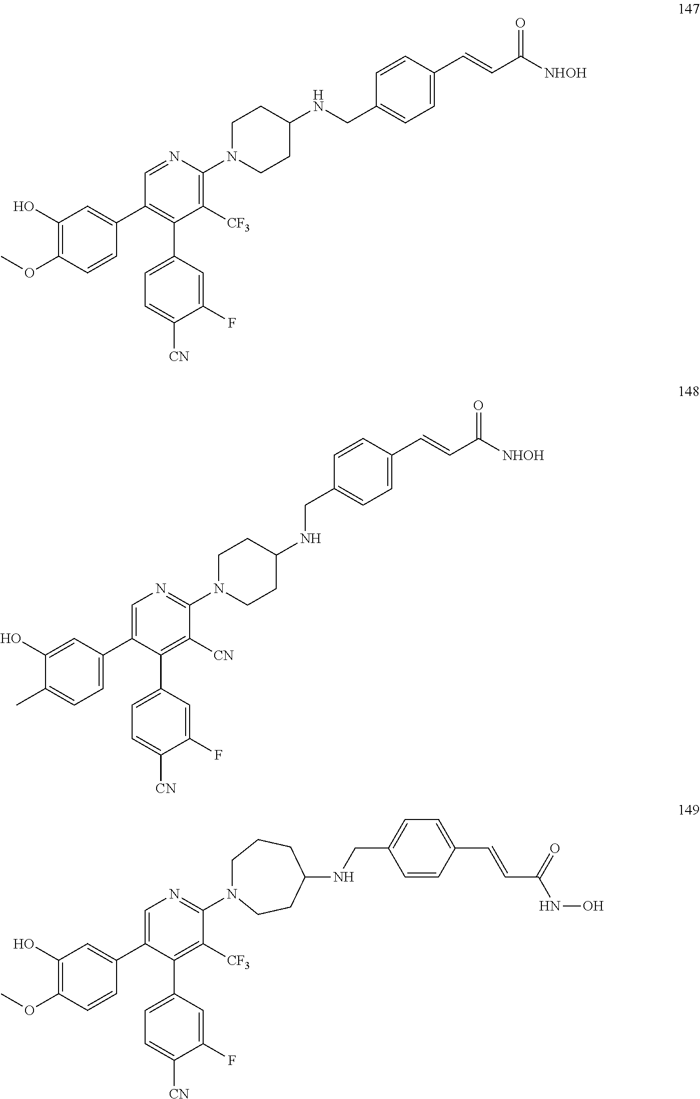

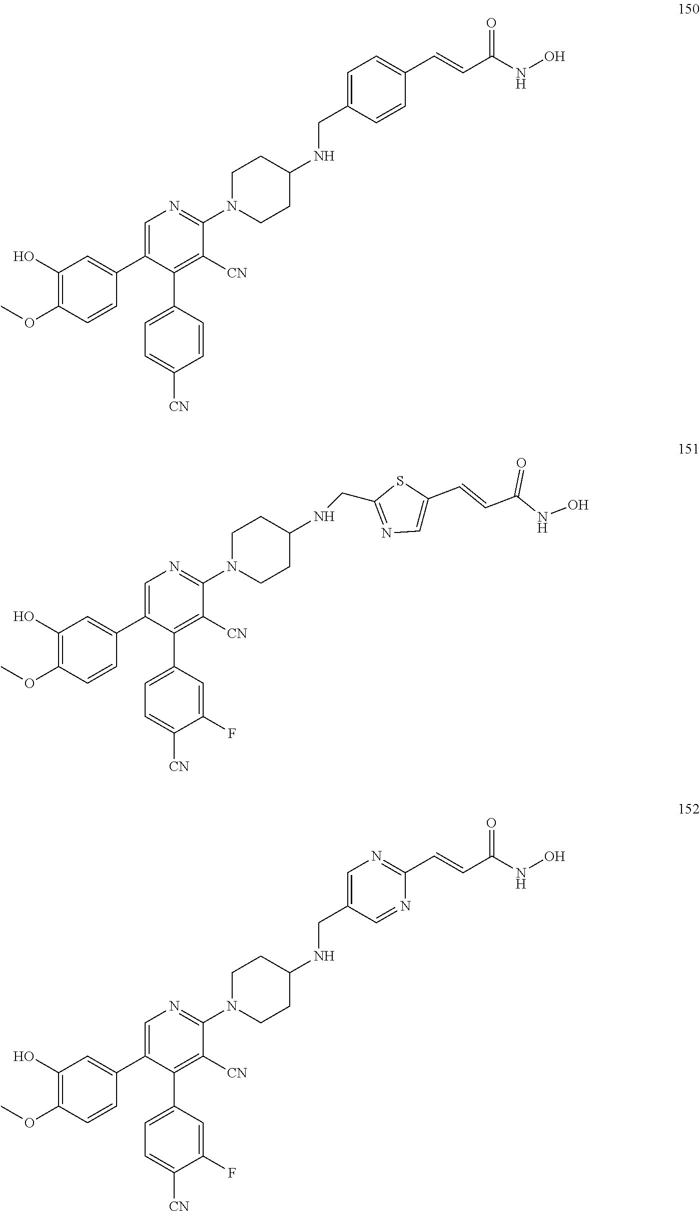 [SMILES: COc1ccc(-c2cnc(N3CCC(NCc4ccc(/C=C/C(=O)NO)cc4)CC3)c(C#N)c2-c2ccc(C#N)cc2)cc1O ; COc1ccc(-c2cnc(N3CCC(NCc4ncc(/C=C/C(=O)NO)s4)CC3)c(C#N)c2-c2ccc(C#N)c(F)c2)cc1O ; COc1ccc(-c2cnc(N3CCC(NCc4cnc(/C=C/C(=O)NO)nc4)CC3)c(C#N)c2-c2ccc(C#N)c(F)c2)cc1O]

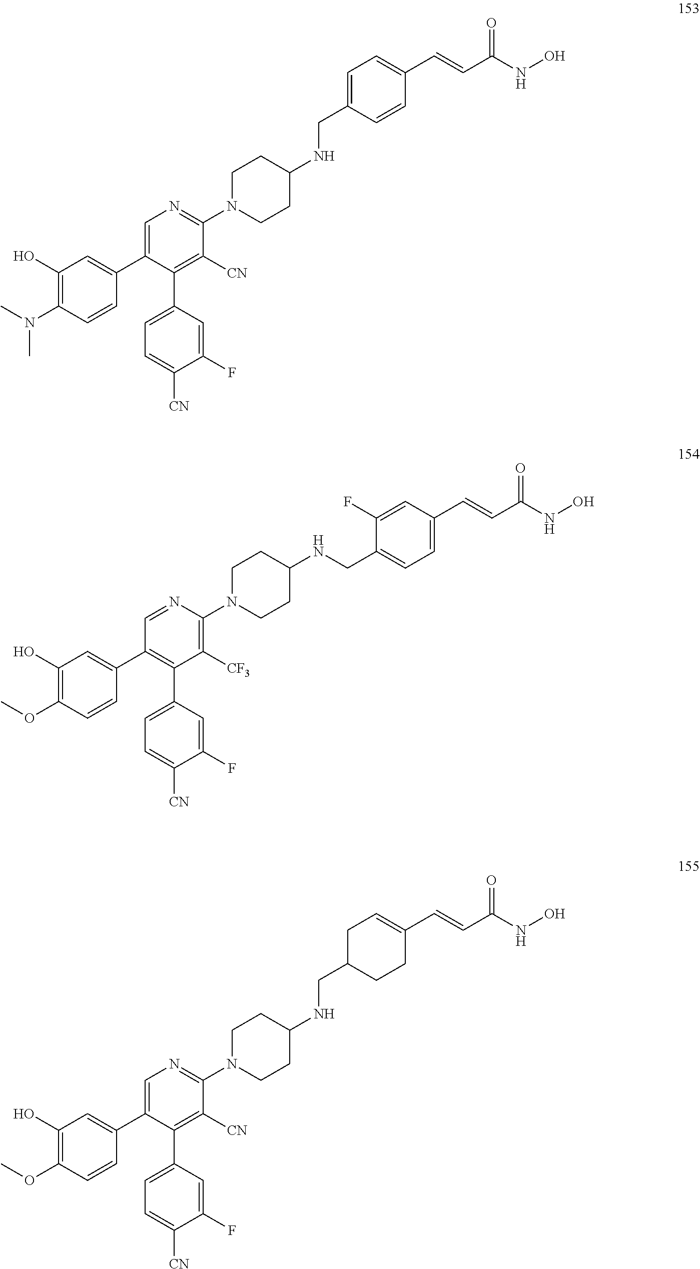 [SMILES: CN(C)c1ccc(-c2cnc(N3CCC(NCc4ccc(/C=C/C(=O)NO)cc4)CC3)c(C#N)c2-c2ccc(C#N)c(F)c2)cc1O ; COc1ccc(-c2cnc(N3CCC(NCc4ccc(/C=C/C(=O)NO)cc4F)CC3)c(C(F)(F)F)c2-c2ccc(C#N)c(F)c2)cc1O ; COc1ccc(-c2cnc(N3CCC(NCC4CC=C(/C=C/C(=O)NO)CC4)CC3)c(C#N)c2-c2ccc(C#N)c(F)c2)cc1O]

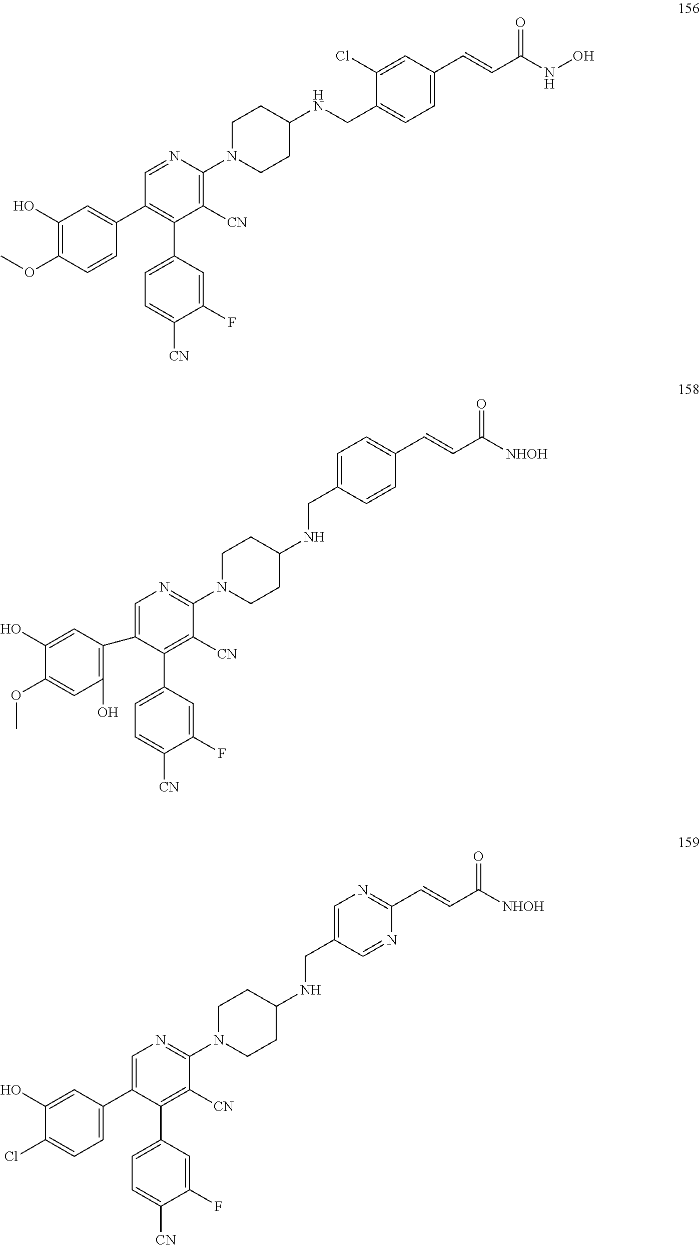

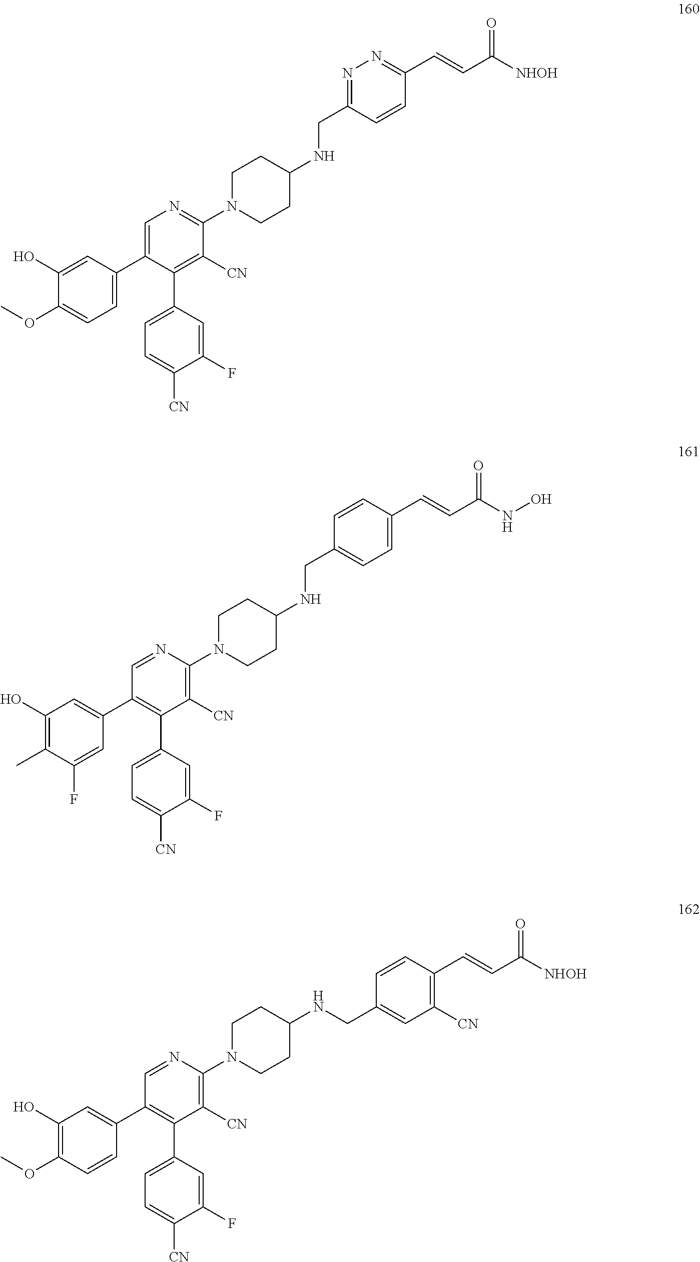

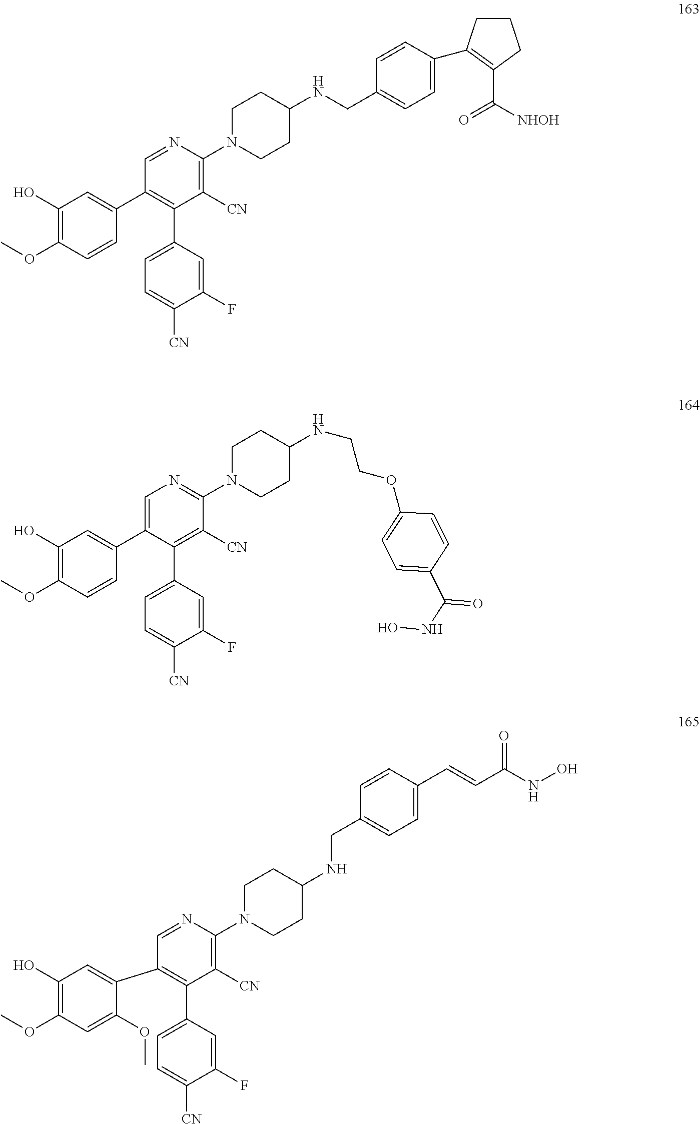

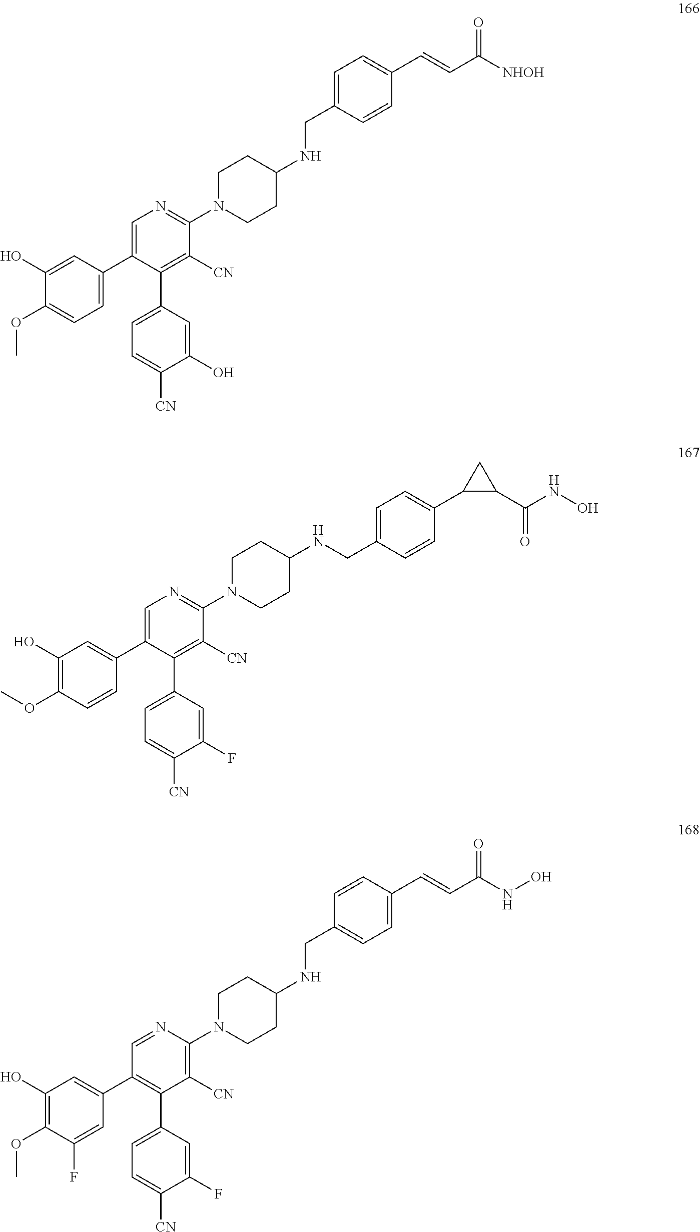

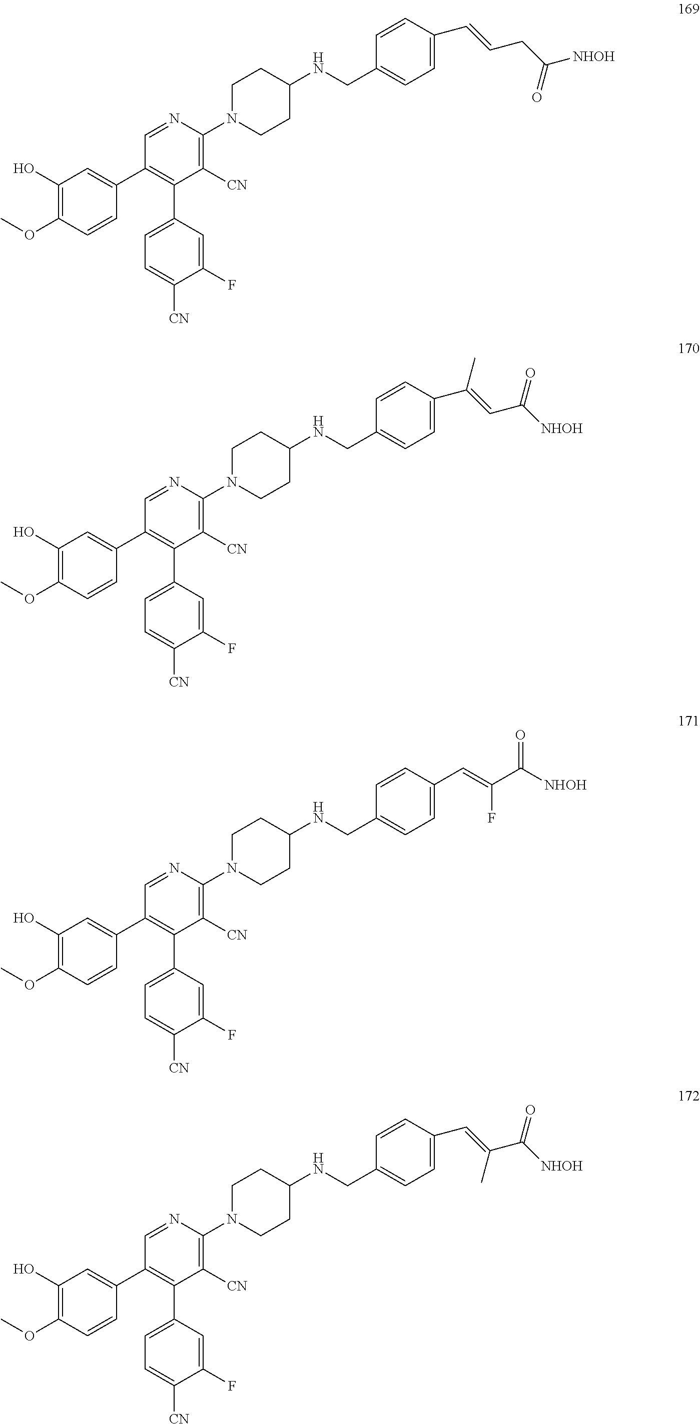

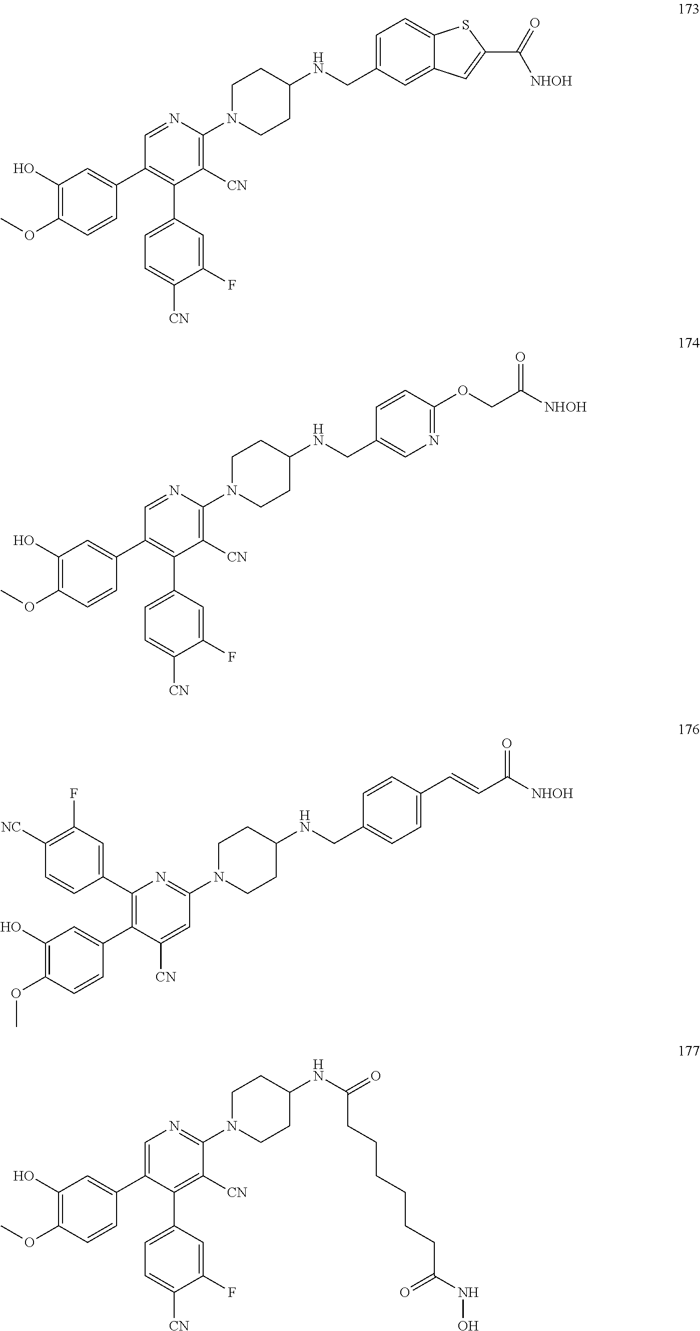

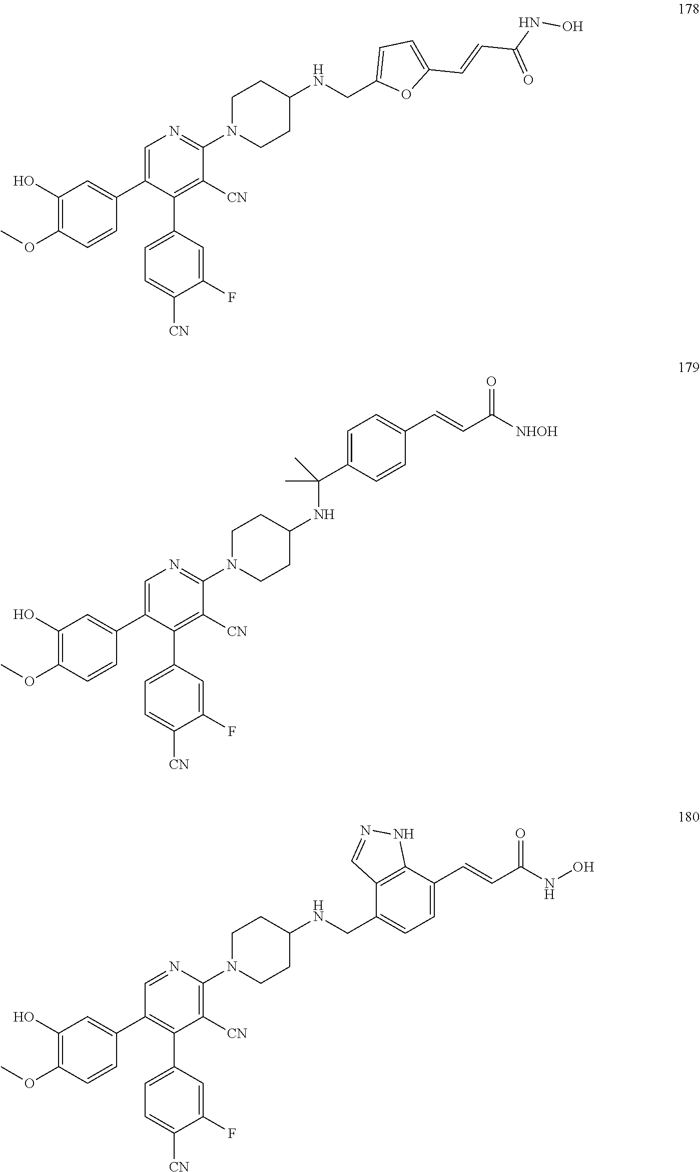

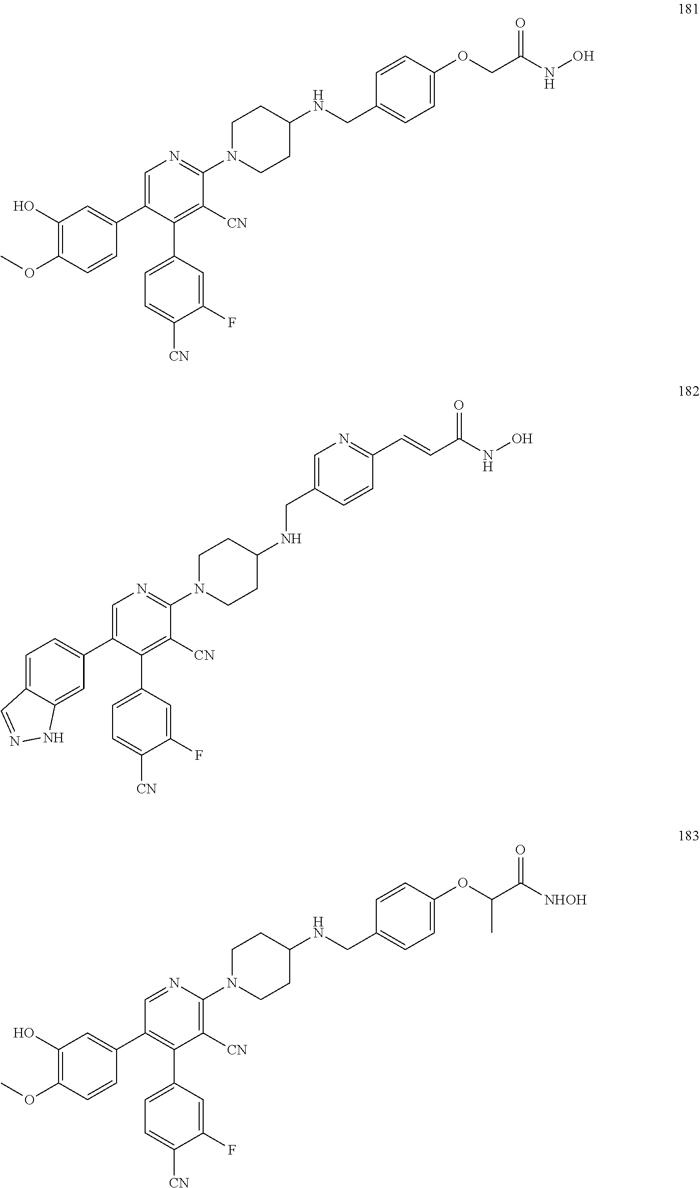

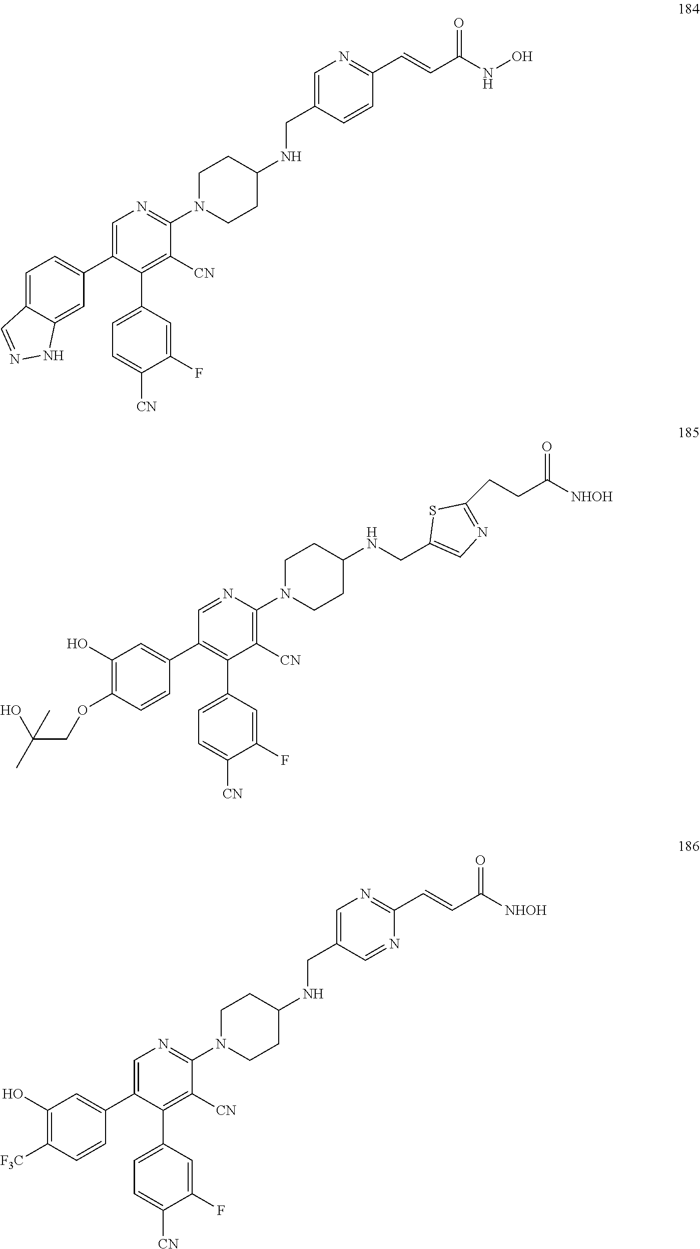

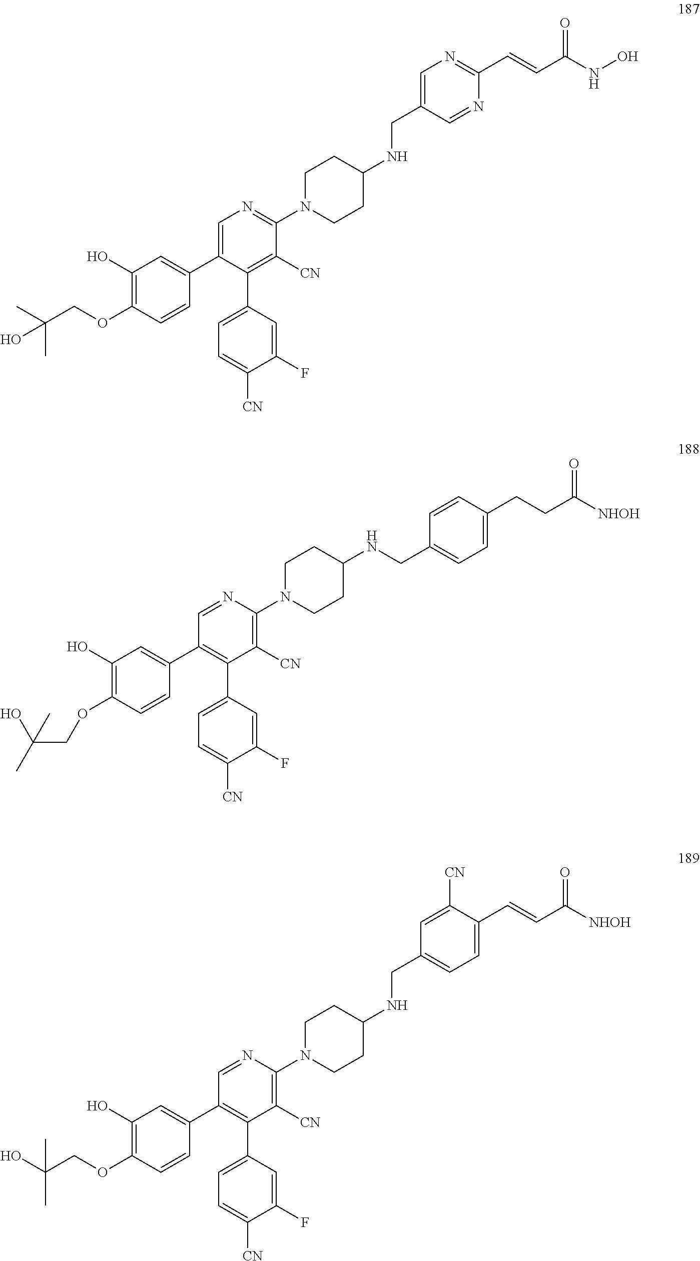

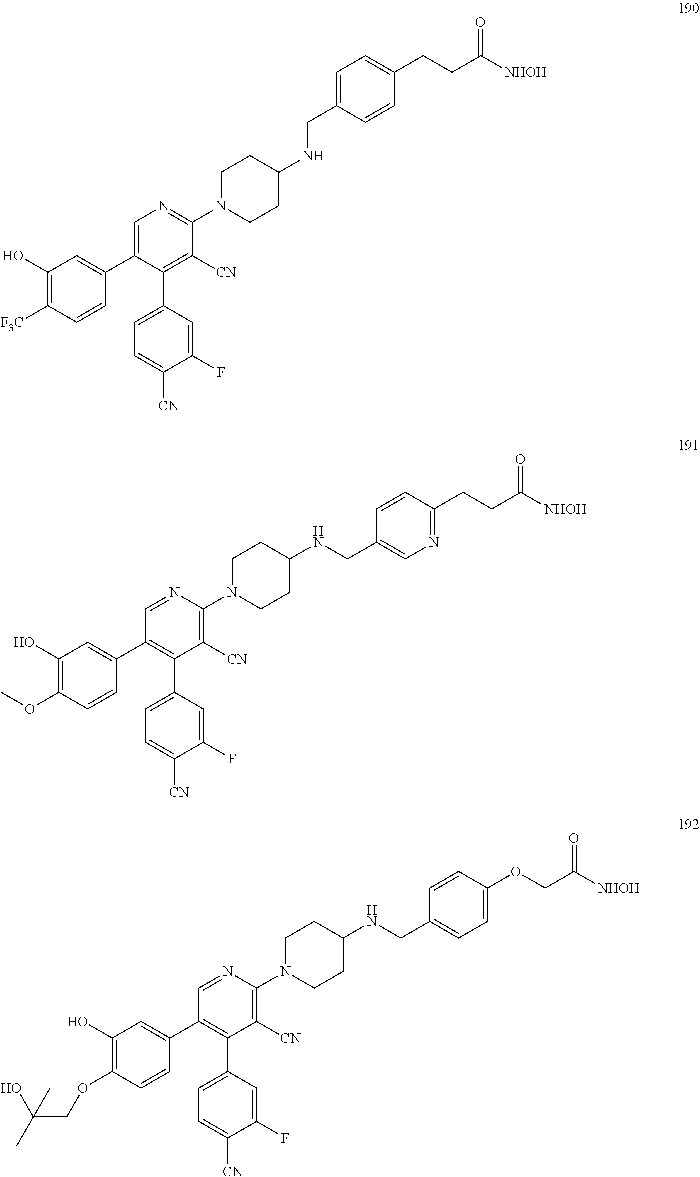

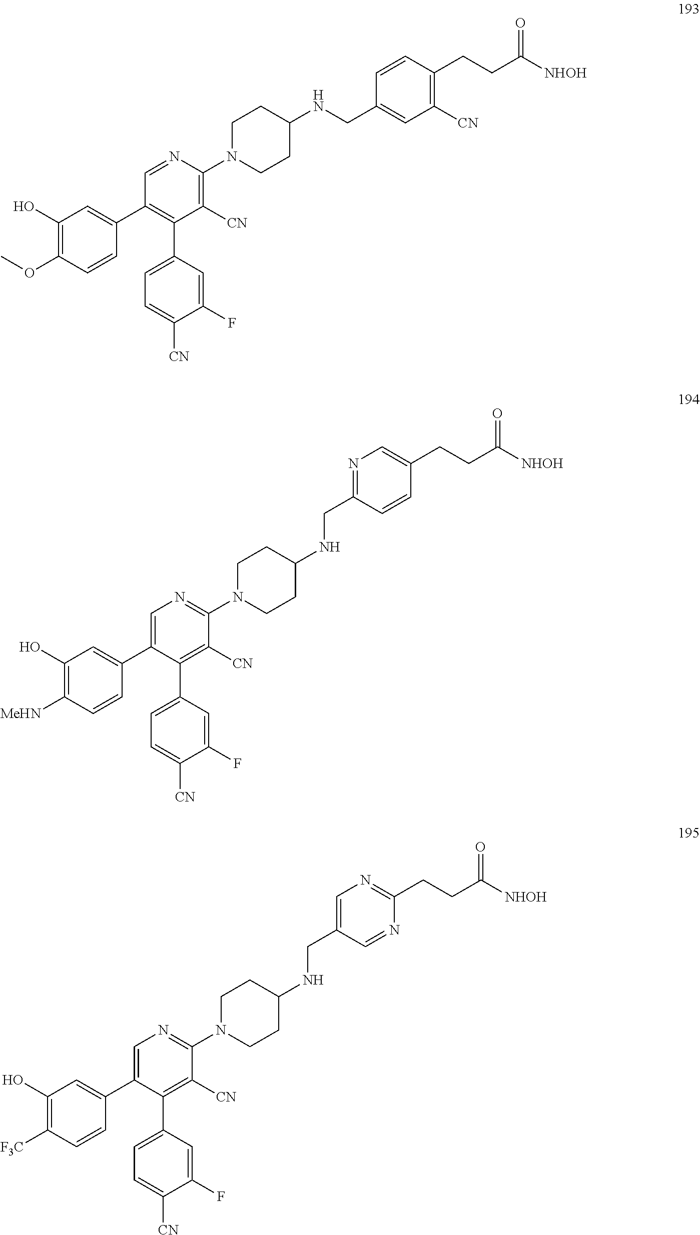

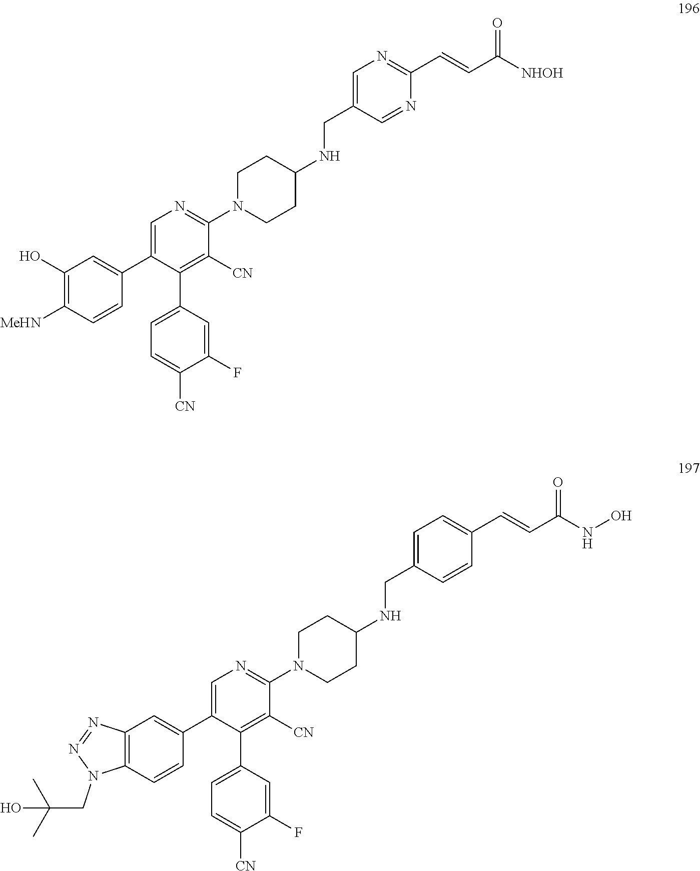

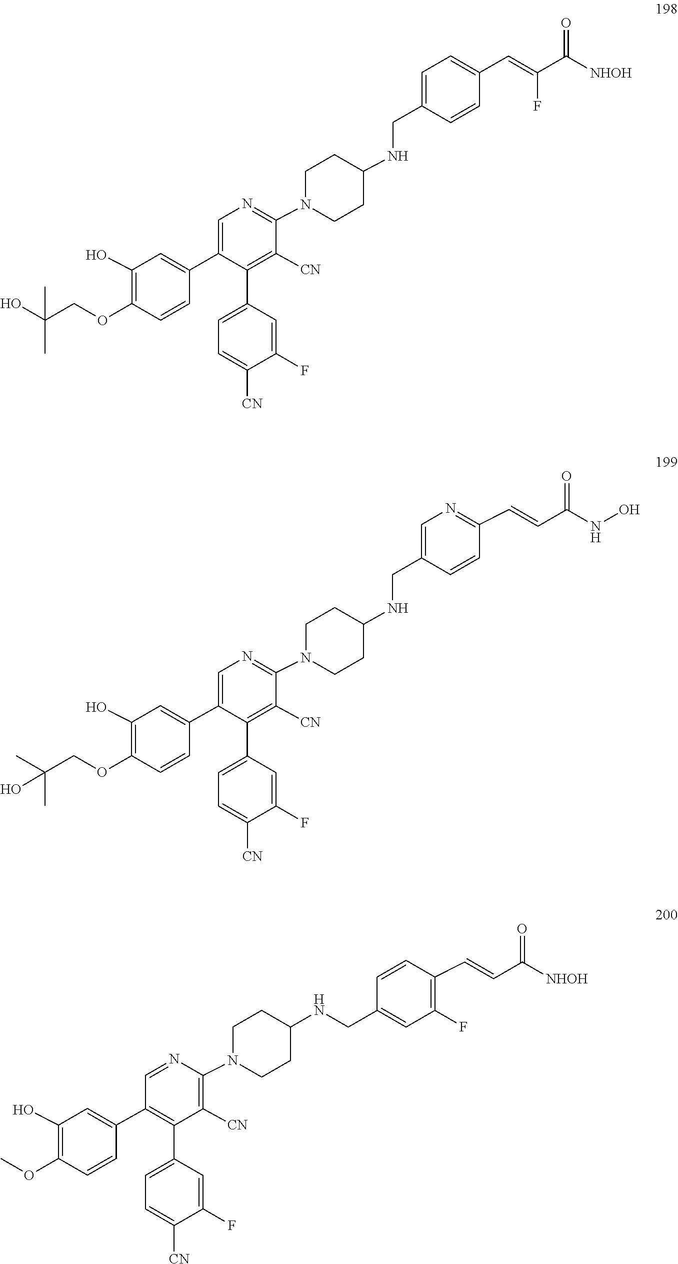

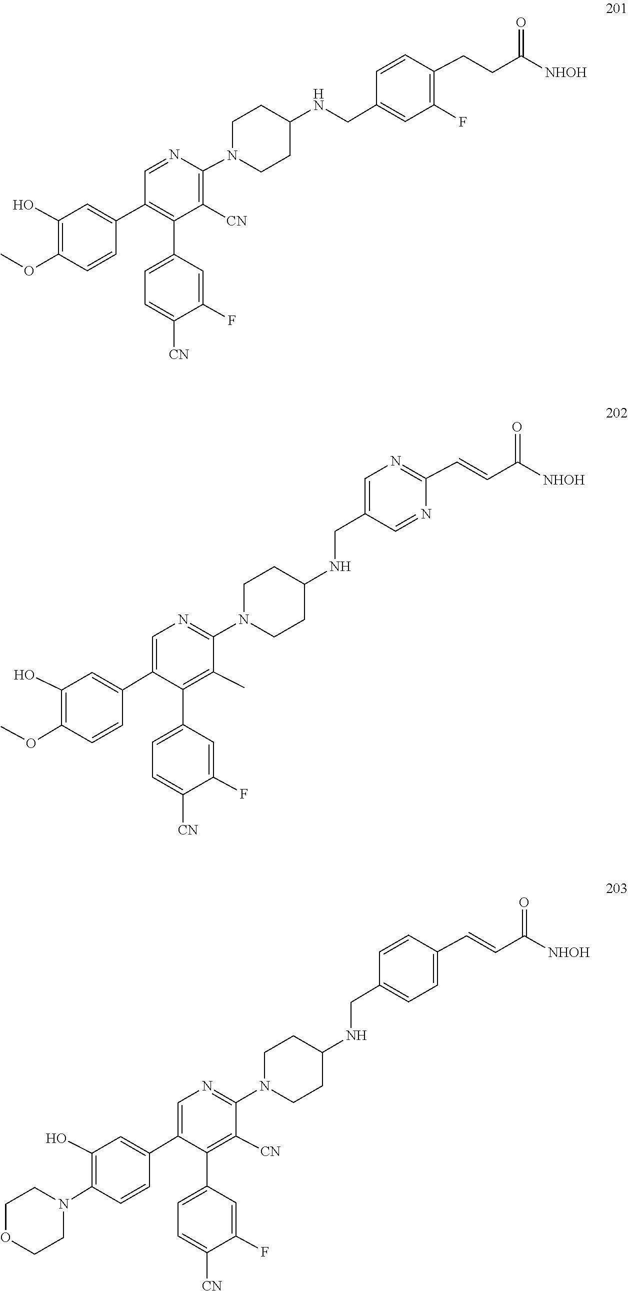

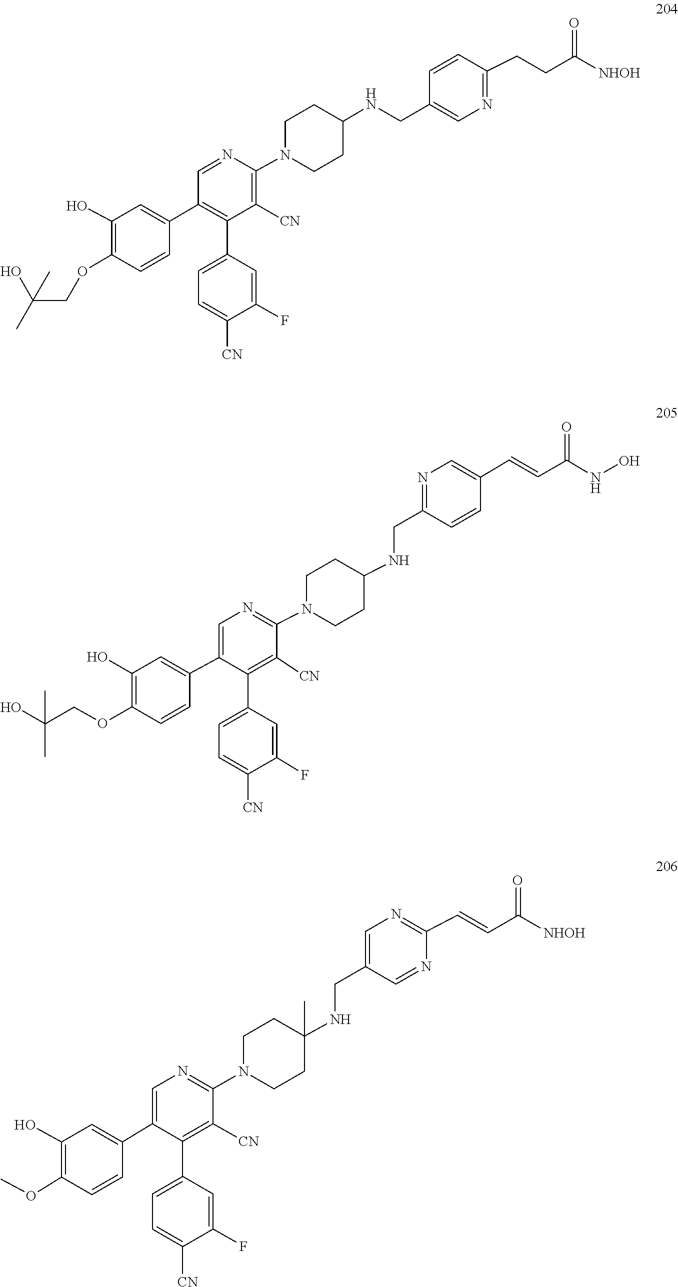

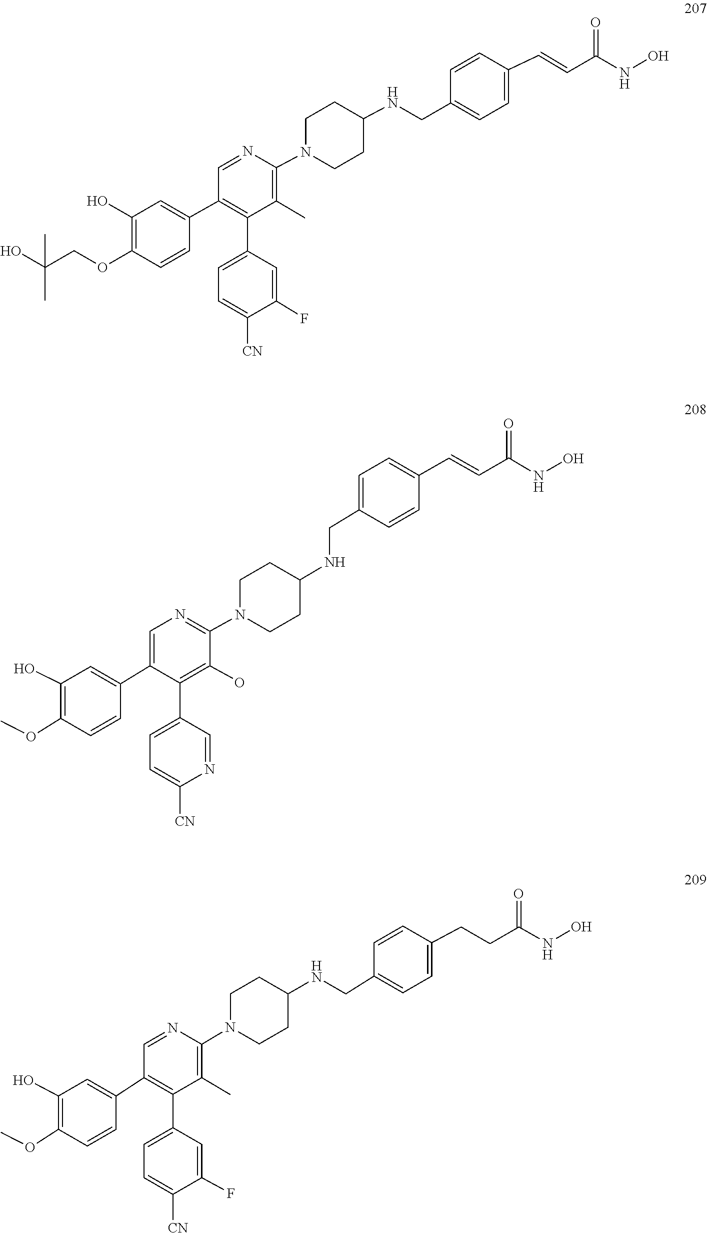 [SMILES: Cc1c(N2CCC(NCc3ccc(/C=C/C(=O)NO)cc3)CC2)ncc(-c2ccc(OCC(C)(C)O)c(O)c2)c1-c1ccc(C#N)c(F)c1 ; COc1ccc(-c2cnc(N3CCC(NCc4ccc(/C=C/C(=O)NO)cc4)CC3)c(O)c2-c2ccc(C#N)nc2)cc1O ; COc1ccc(-c2cnc(N3CCC(NCc4ccc(CCC(=O)NO)cc4)CC3)c(C)c2-c2ccc(C#N)c(F)c2)cc1O]

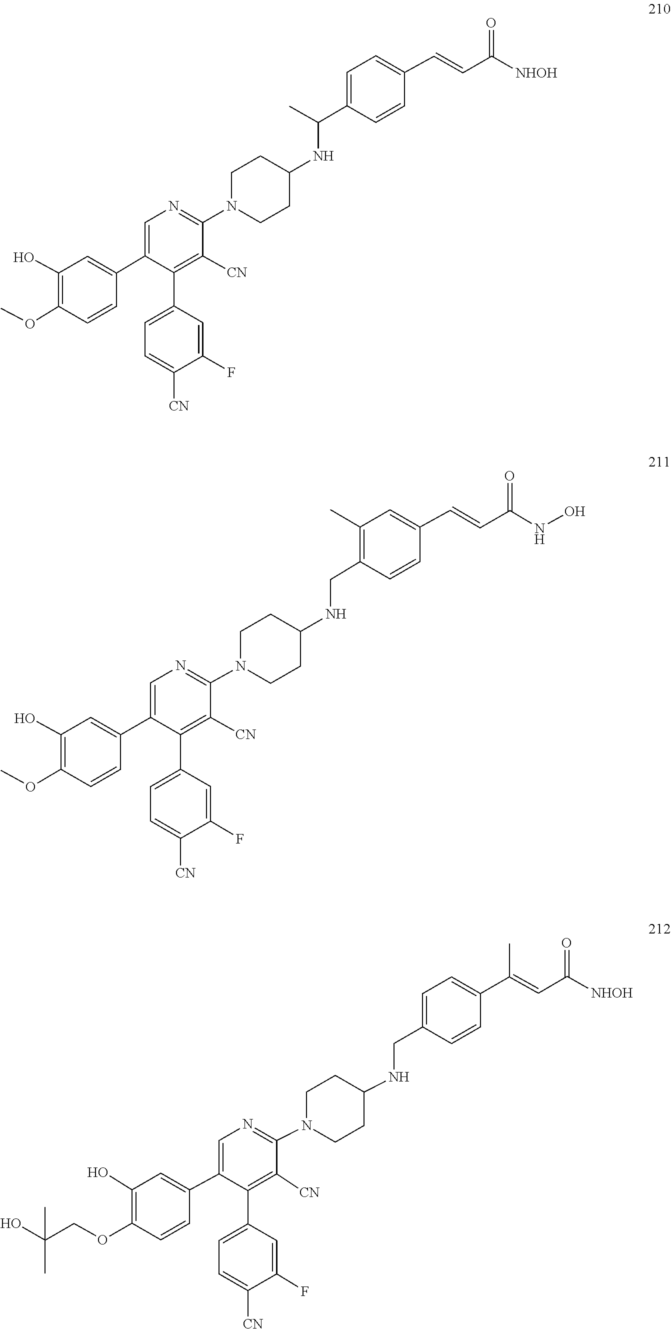

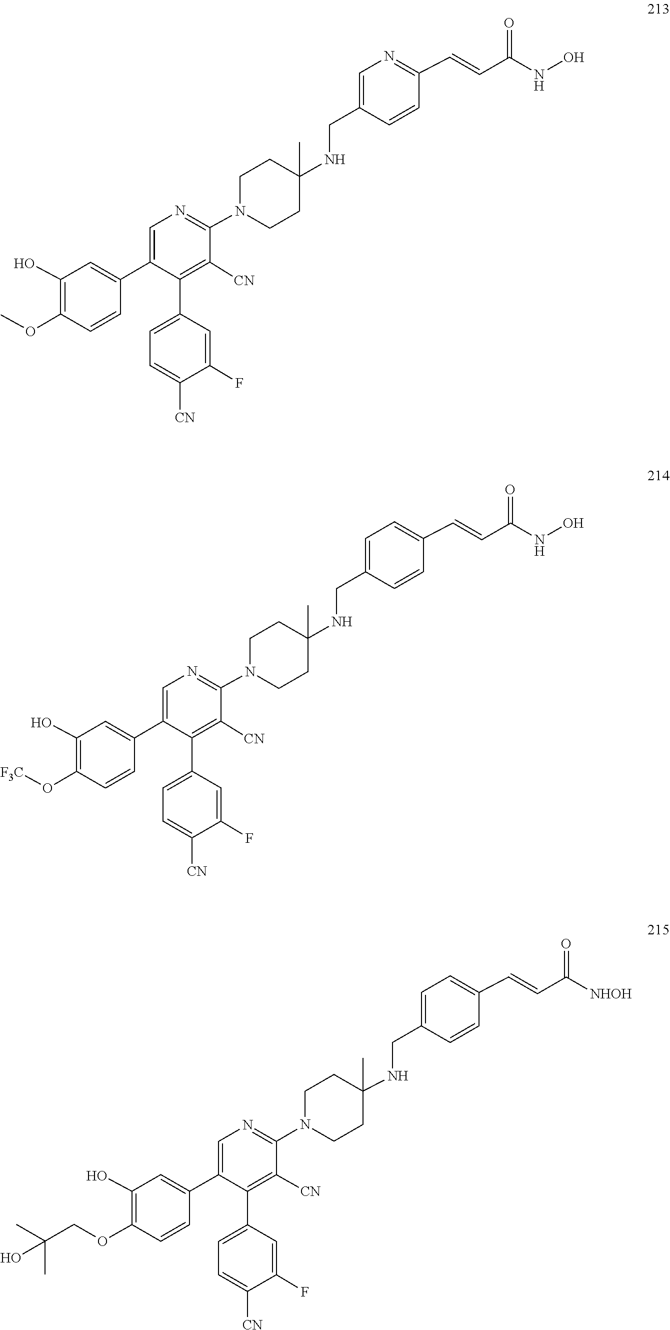

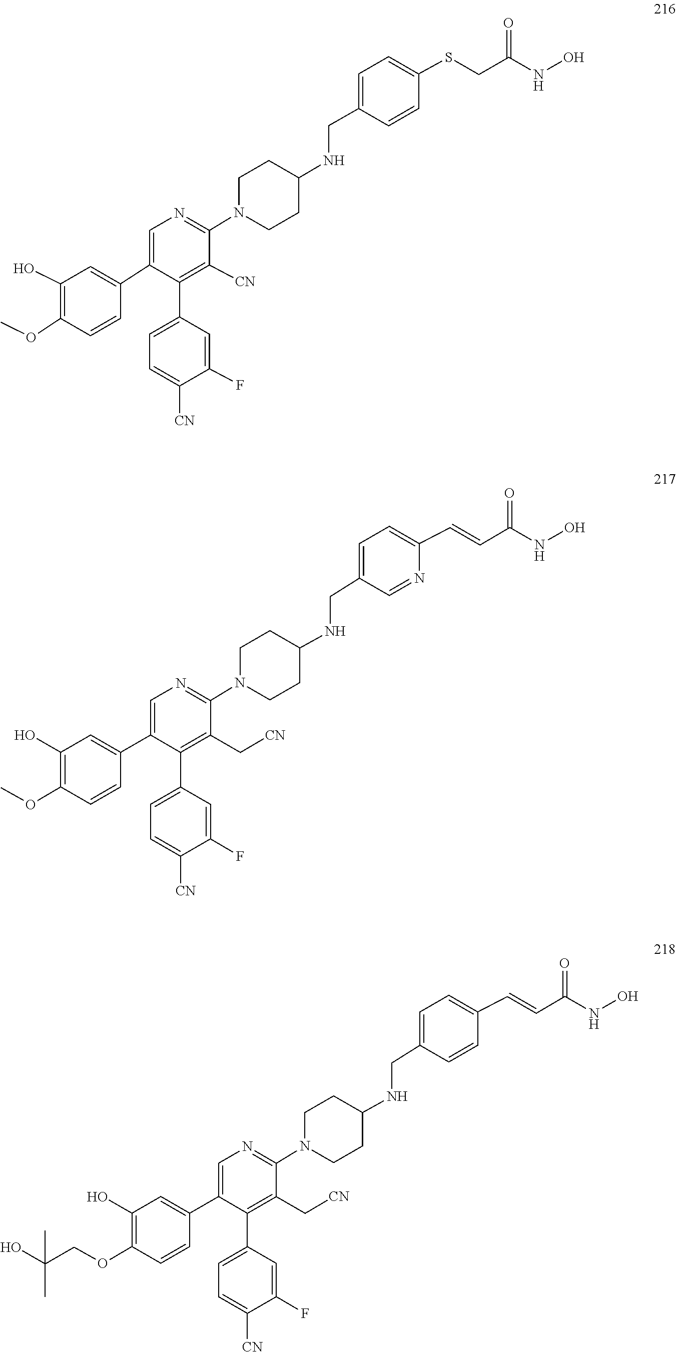 [SMILES: COc1ccc(-c2cnc(N3CCC(NCc4ccc(SCC(=O)NO)cc4)CC3)c(C#N)c2-c2ccc(C#N)c(F)c2)cc1O ; COc1ccc(-c2cnc(N3CCC(NCc4ccc(/C=C/C(=O)NO)nc4)CC3)c(CC#N)c2-c2ccc(C#N)c(F)c2)cc1O ; CC(C)(O)COc1ccc(-c2cnc(N3CCC(NCc4ccc(/C=C/C(=O)NO)cc4)CC3)c(CC#N)c2-c2ccc(C#N)c(F)c2)cc1O]

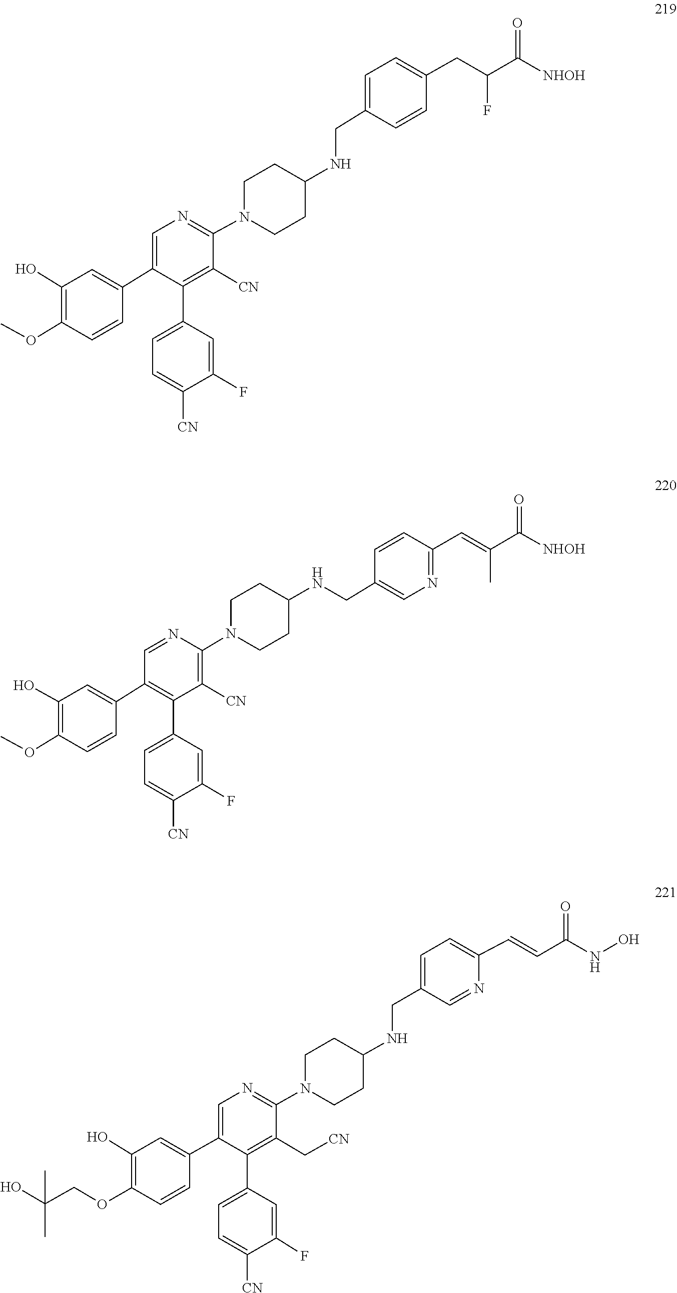

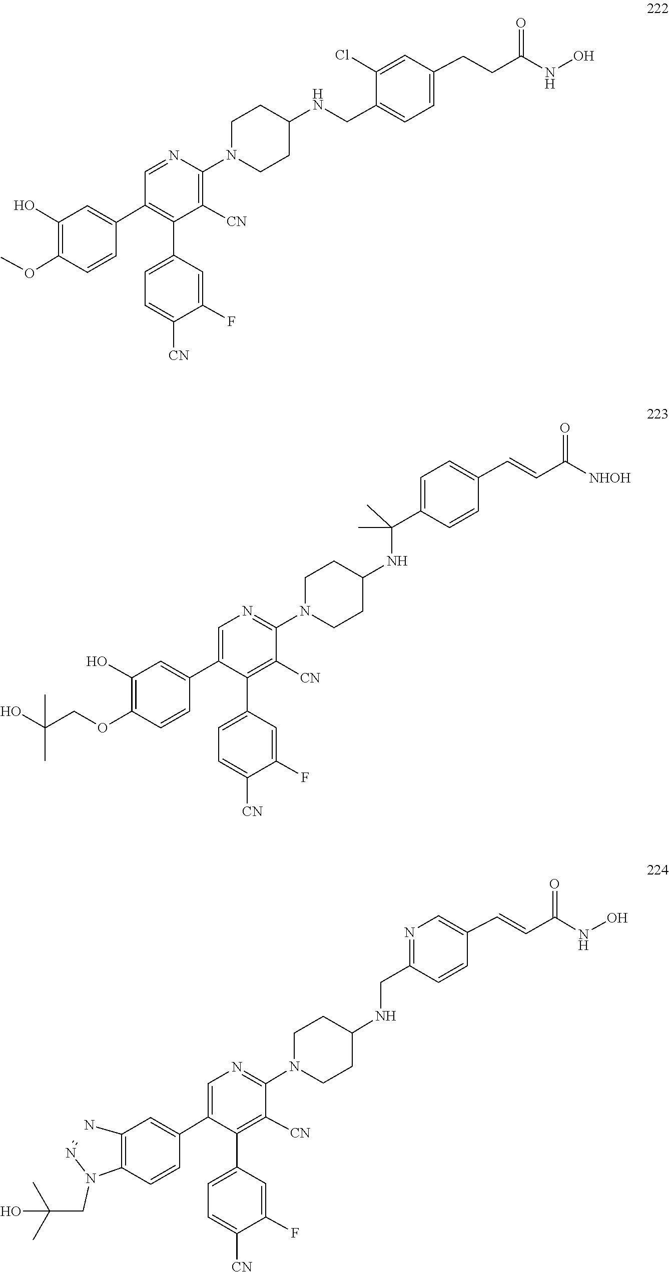

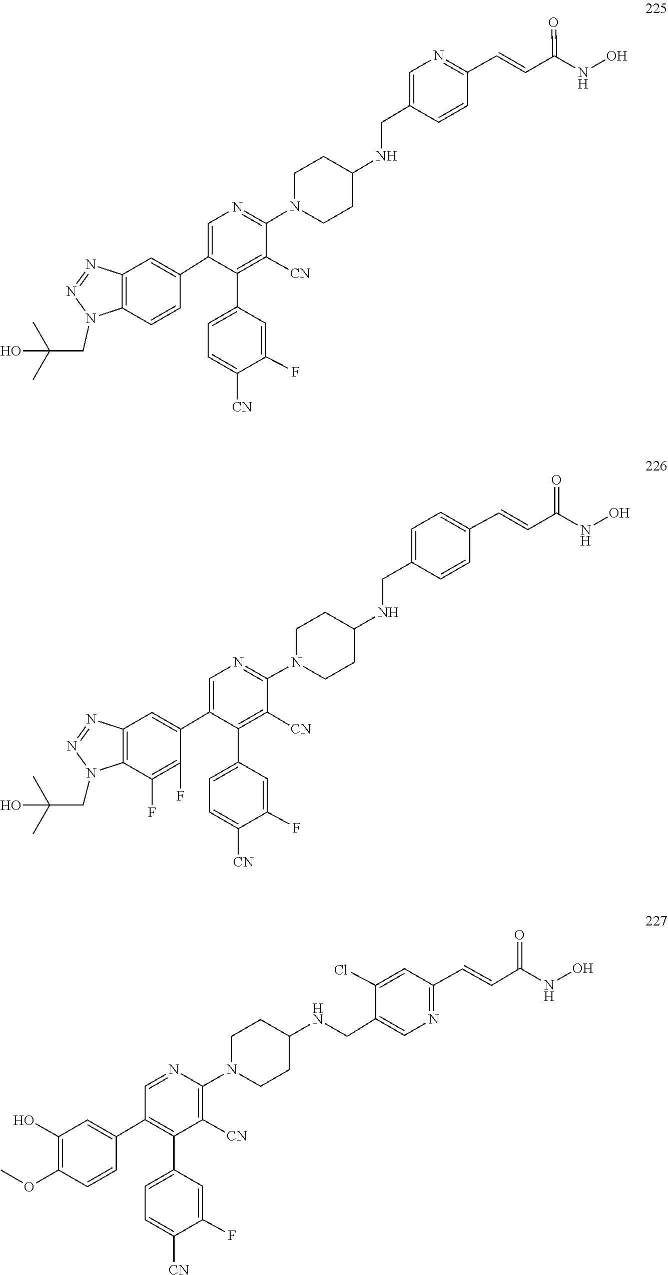 [SMILES: CC(C)(O)Cn1nnc2cc(-c3cnc(N4CCC(NCc5ccc(/C=C/C(=O)NO)nc5)CC4)c(C#N)c3-c3ccc(C#N)c(F)c3)ccc21 ; CC(C)(O)Cn1nnc2cc(-c3cnc(N4CCC(NCc5ccc(/C=C/C(=O)NO)cc5)CC4)c(C#N)c3-c3ccc(C#N)c(F)c3)c(F)c(F)c21 ; COc1ccc(-c2cnc(N3CCC(NCc4cnc(/C=C/C(=O)NO)cc4Cl)CC3)c(C#N)c2-c2ccc(C#N)c(F)c2)cc1O]

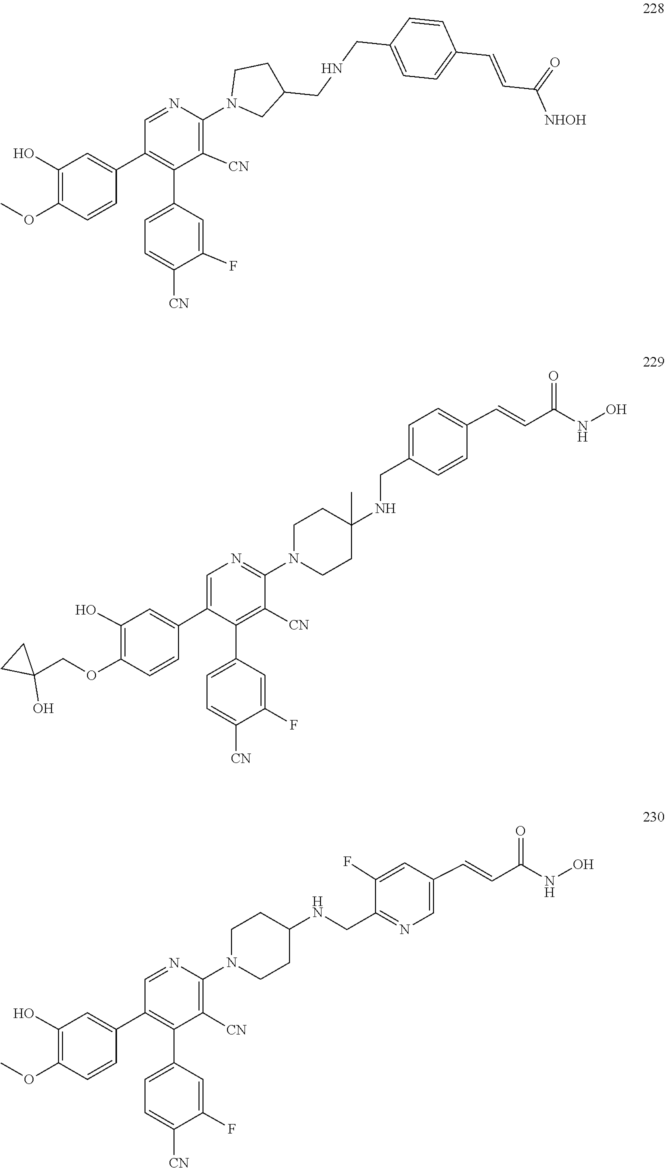

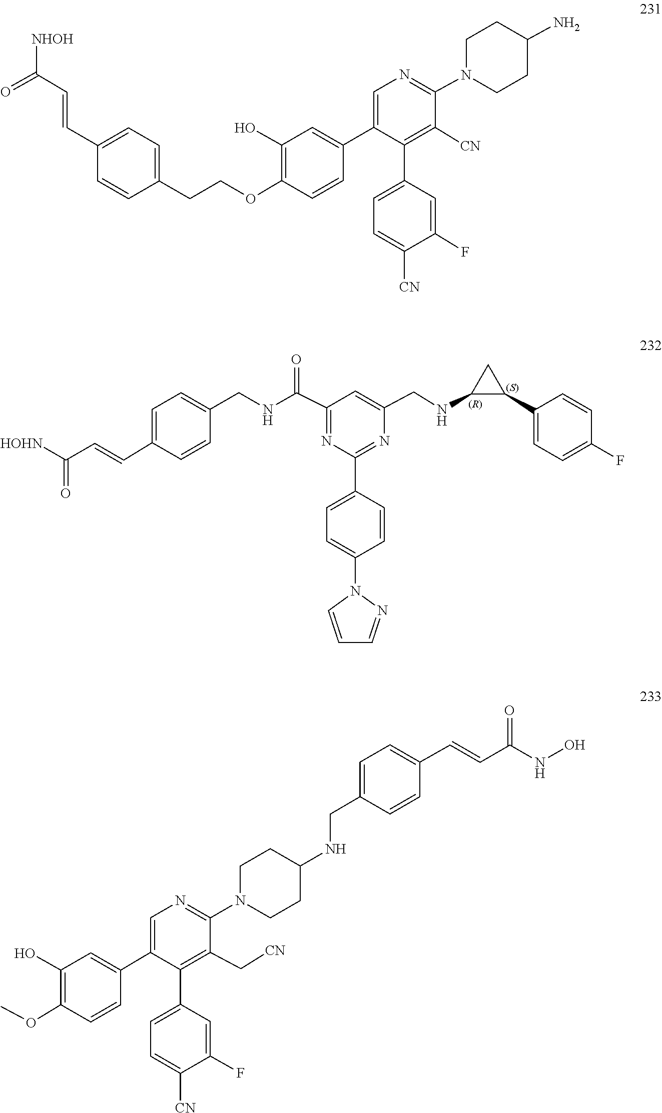

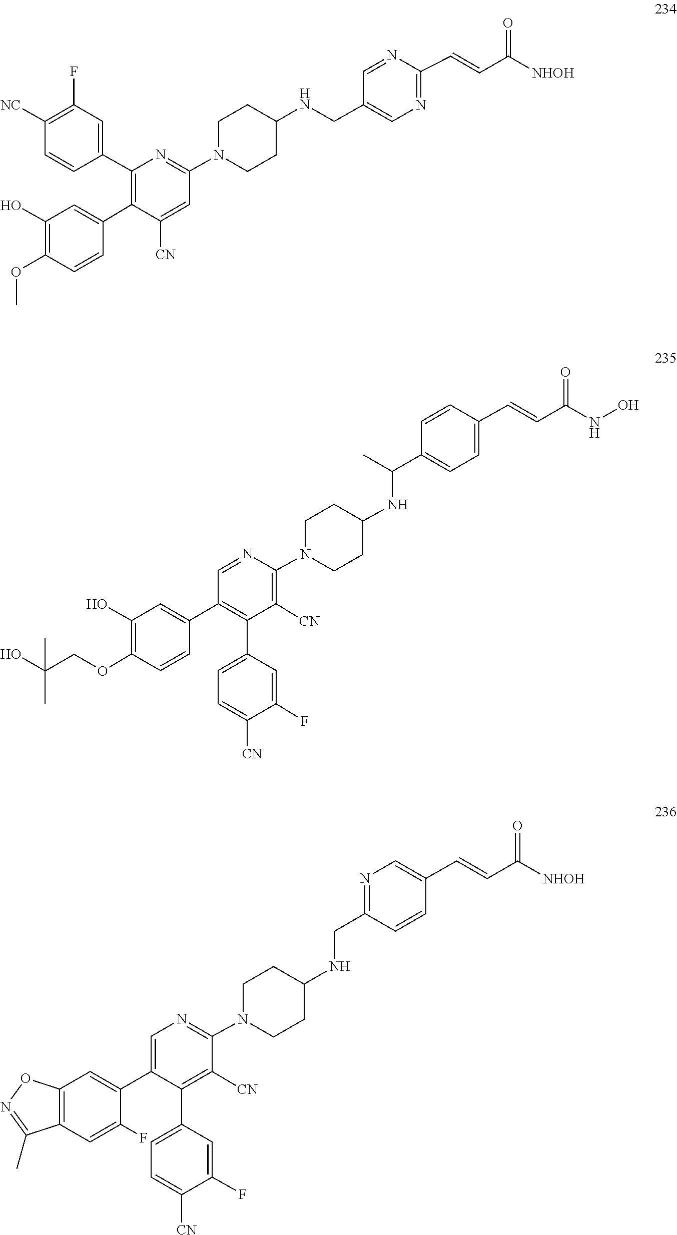

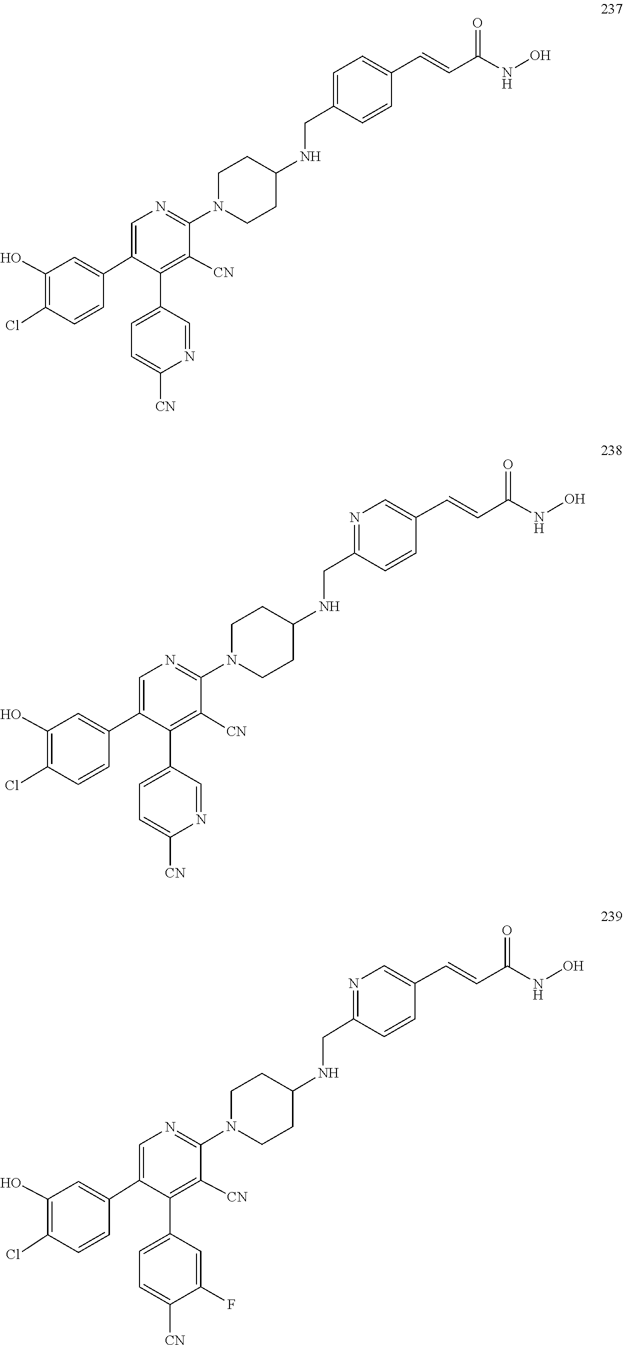 [SMILES: N#Cc1ccc(-c2c(-c3ccc(Cl)c(O)c3)cnc(N3CCC(NCc4ccc(/C=C/C(=O)NO)cc4)CC3)c2C#N)cn1 ; N#Cc1ccc(-c2c(-c3ccc(Cl)c(O)c3)cnc(N3CCC(NCc4ccc(/C=C/C(=O)NO)cn4)CC3)c2C#N)cn1 ; N#Cc1ccc(-c2c(-c3ccc(Cl)c(O)c3)cnc(N3CCC(NCc4ccc(/C=C/C(=O)NO)cn4)CC3)c2C#N)cc1F]

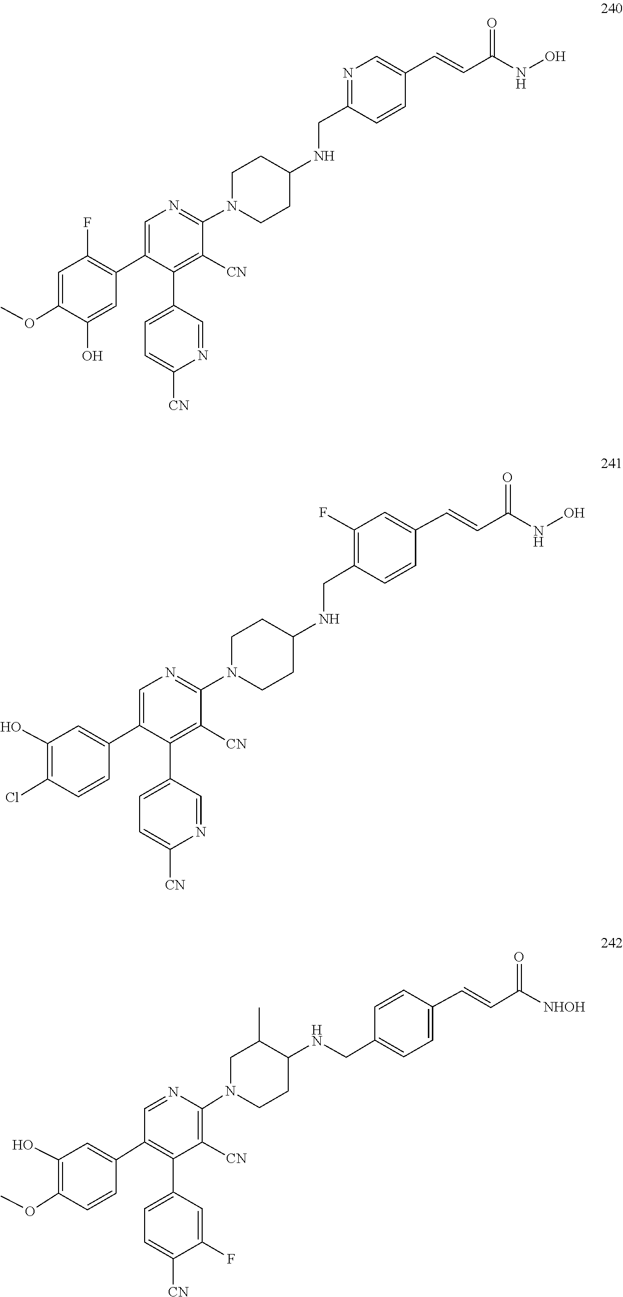

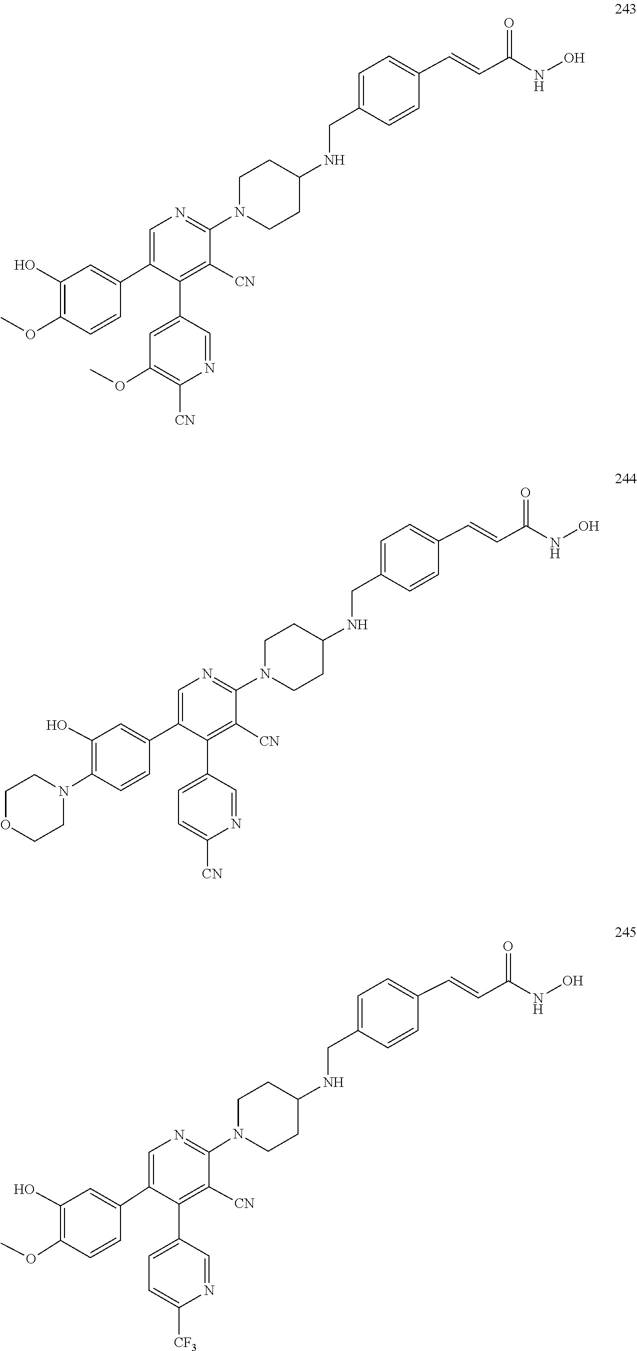 [SMILES: COc1ccc(-c2cnc(N3CCC(NCc4ccc(/C=C/C(=O)NO)cc4)CC3)c(C#N)c2-c2cnc(C#N)c(OC)c2)cc1O ; N#Cc1ccc(-c2c(-c3ccc(N4CCOCC4)c(O)c3)cnc(N3CCC(NCc4ccc(/C=C/C(=O)NO)cc4)CC3)c2C#N)cn1 ; COc1ccc(-c2cnc(N3CCC(NCc4ccc(/C=C/C(=O)NO)cc4)CC3)c(C#N)c2-c2ccc(C(F)(F)F)nc2)cc1O]

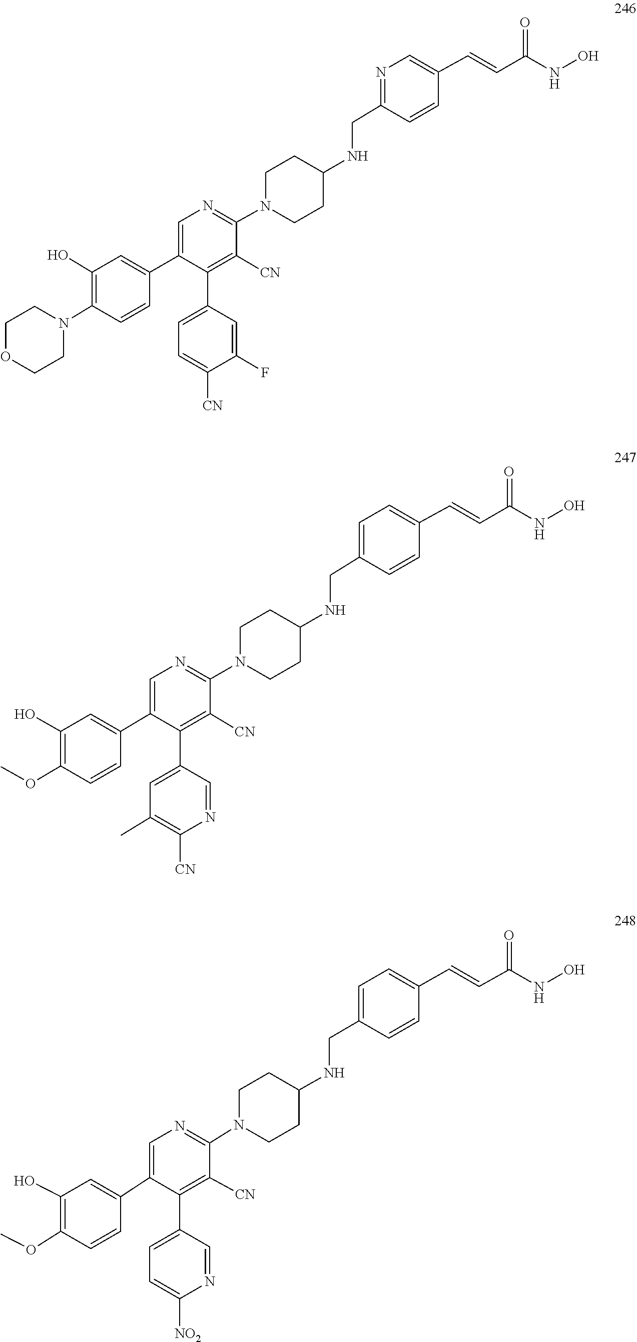 [SMILES: N#Cc1ccc(-c2c(-c3ccc(N4CCOCC4)c(O)c3)cnc(N3CCC(NCc4ccc(/C=C/C(=O)NO)cn4)CC3)c2C#N)cc1F ; COc1ccc(-c2cnc(N3CCC(NCc4ccc(/C=C/C(=O)NO)cc4)CC3)c(C#N)c2-c2cnc(C#N)c(C)c2)cc1O ; COc1ccc(-c2cnc(N3CCC(NCc4ccc(/C=C/C(=O)NO)cc4)CC3)c(C#N)c2-c2ccc([N+](=O)[O-])nc2)cc1O]

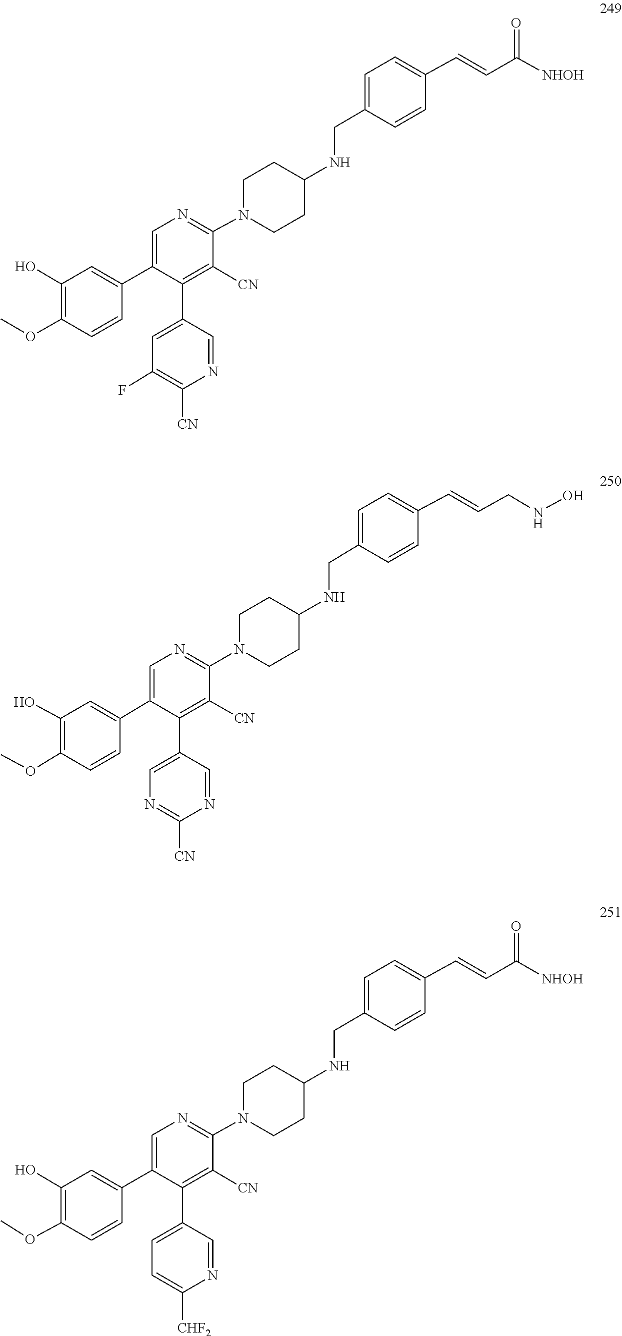

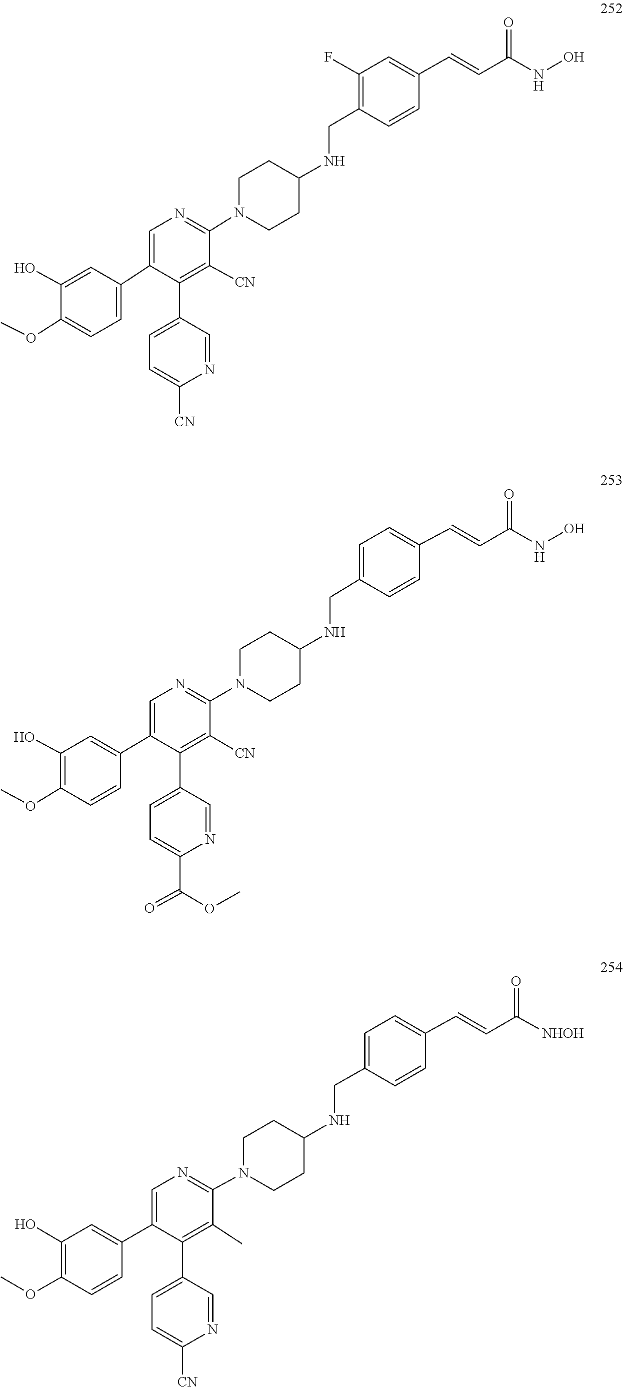

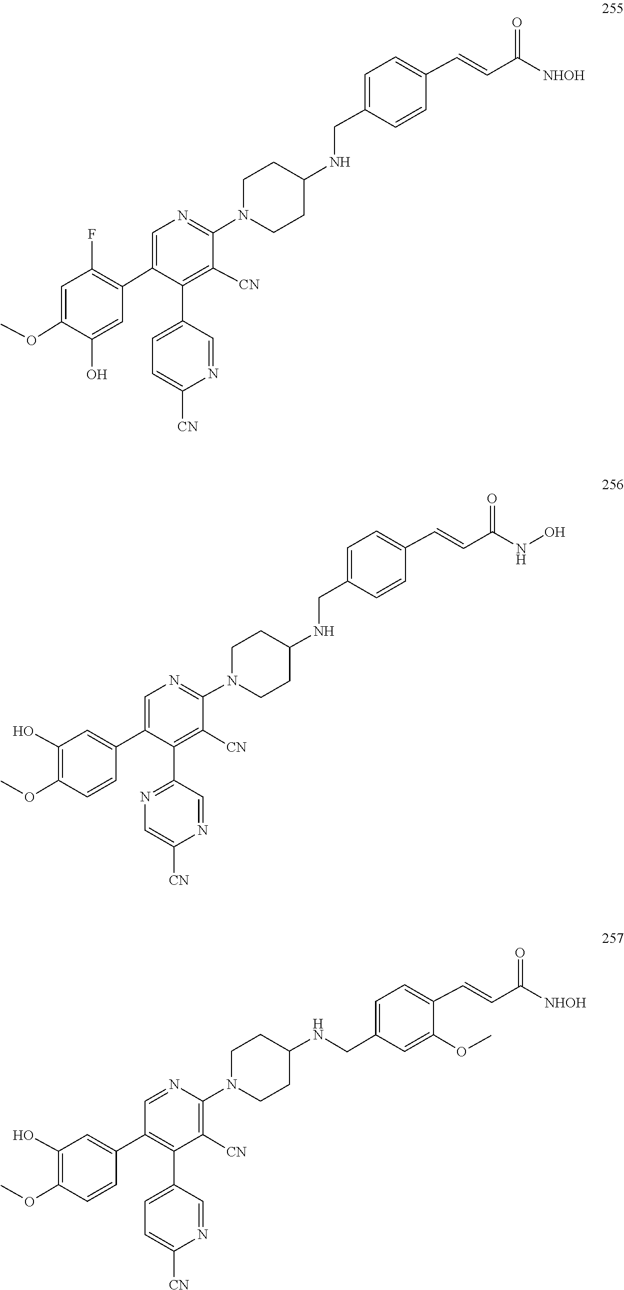

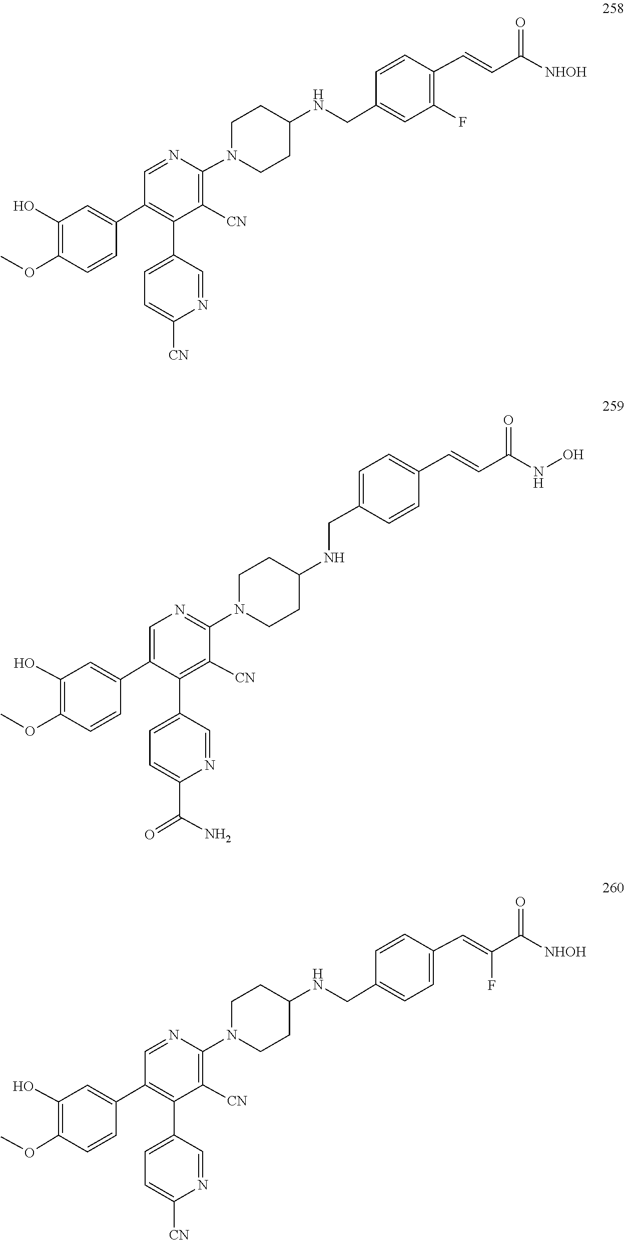

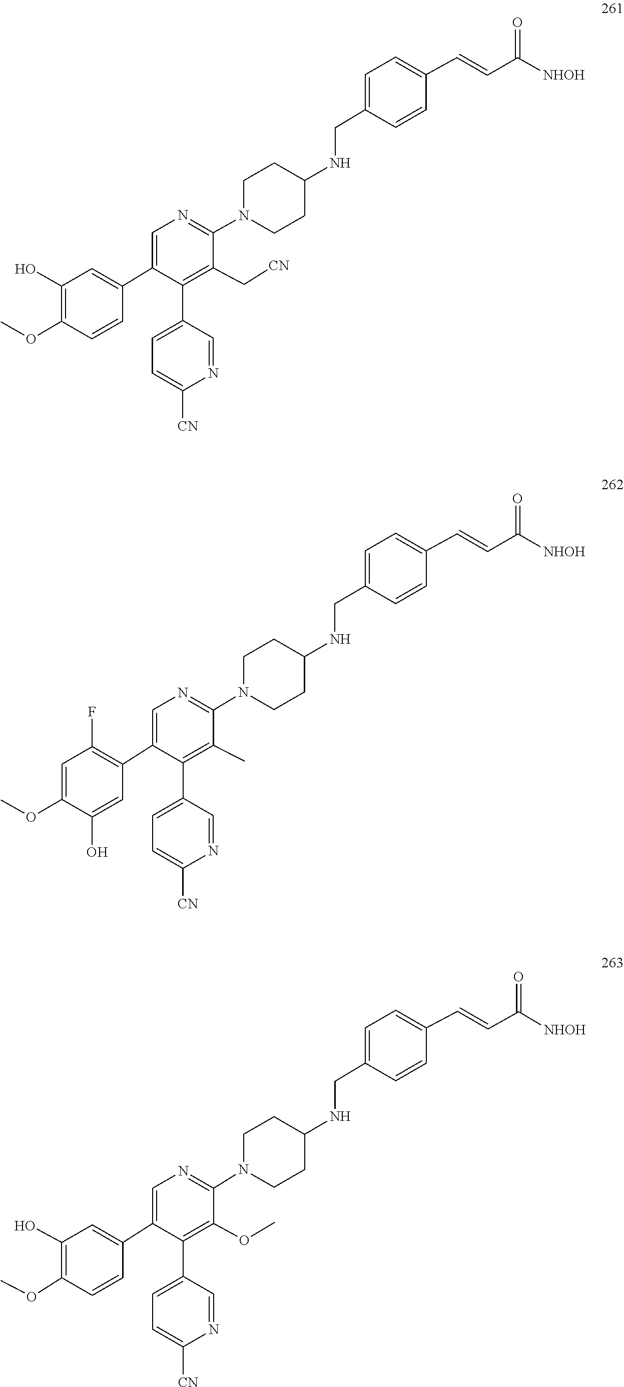

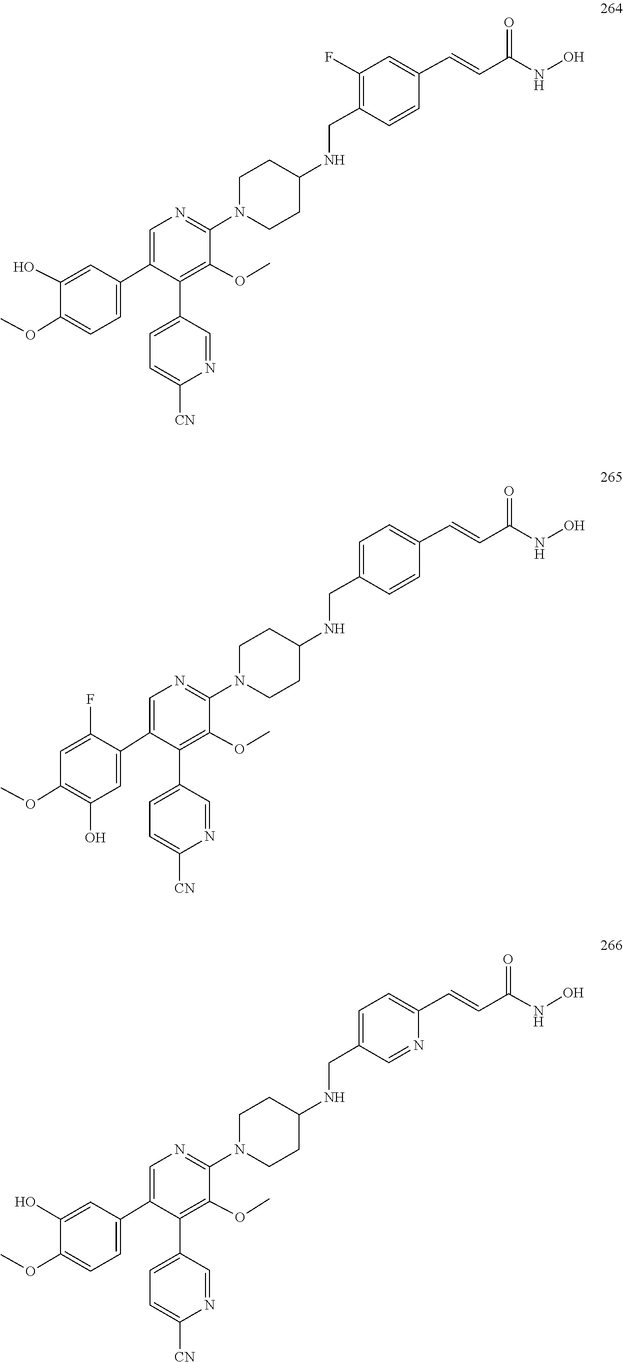 [SMILES: COc1ccc(-c2cnc(N3CCC(NCc4ccc(/C=C/C(=O)NO)cc4F)CC3)c(OC)c2-c2ccc(C#N)nc2)cc1O ; COc1cc(F)c(-c2cnc(N3CCC(NCc4ccc(/C=C/C(=O)NO)cc4)CC3)c(OC)c2-c2ccc(C#N)nc2)cc1O ; COc1ccc(-c2cnc(N3CCC(NCc4ccc(/C=C/C(=O)NO)nc4)CC3)c(OC)c2-c2ccc(C#N)nc2)cc1O]

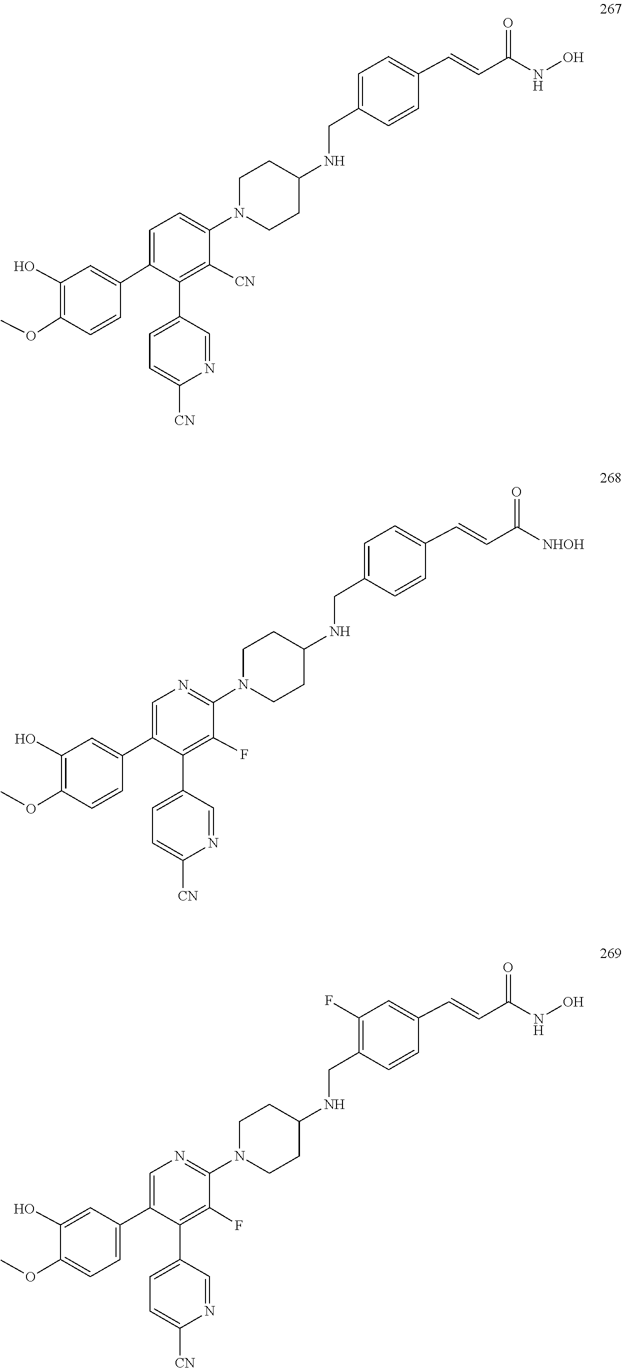

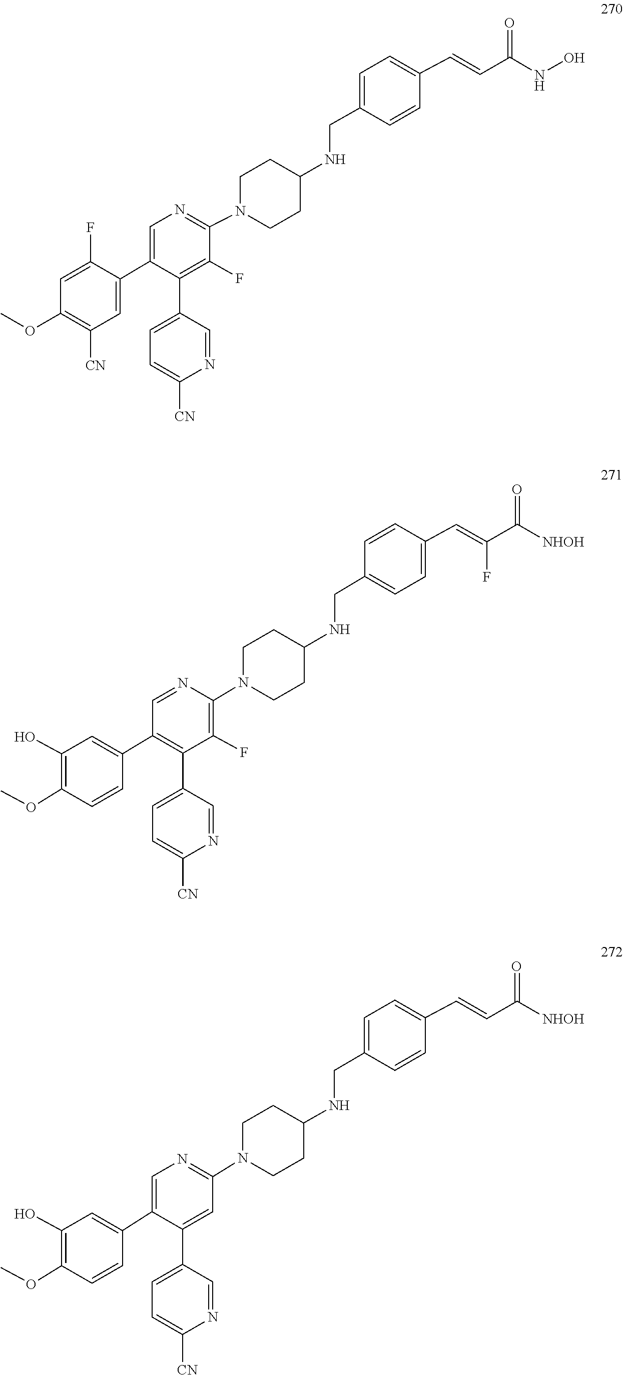

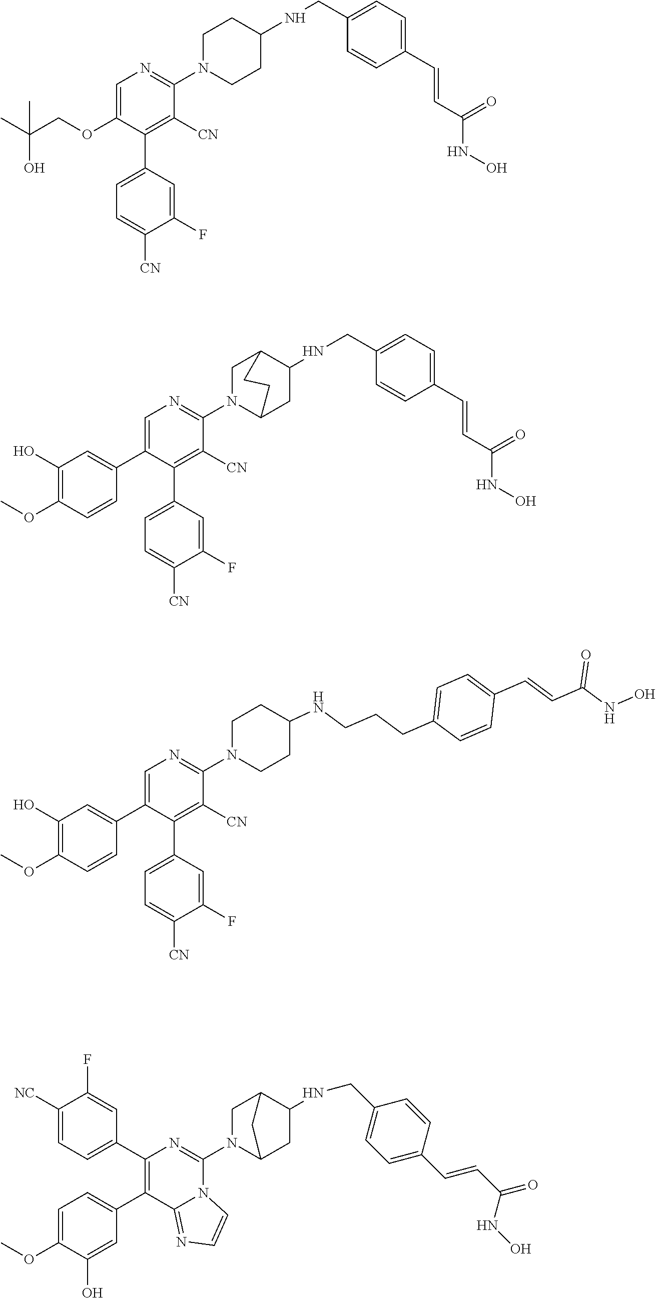 [SMILES: CC(C)(O)COc1cnc(N2CCC(NCc3ccc(/C=C/C(=O)NO)cc3)CC2)c(C#N)c1-c1ccc(C#N)c(F)c1 ; COc1ccc(-c2cnc(N3CC4CCC3CC4NCc3ccc(/C=C/C(=O)NO)cc3)c(C#N)c2-c2ccc(C#N)c(F)c2)cc1O ; COc1ccc(-c2cnc(N3CCC(NCCCc4ccc(/C=C/C(=O)NO)cc4)CC3)c(C#N)c2-c2ccc(C#N)c(F)c2)cc1O ; COc1ccc(-c2c(-c3ccc(C#N)c(F)c3)nc(N3CC4CC3CC4NCc3ccc(/C=C/C(=O)NO)cc3)n3ccnc23)cc1O]

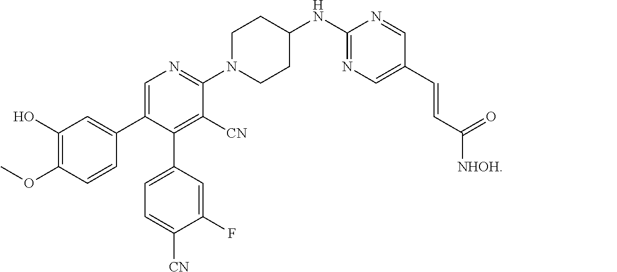

26. A pharmaceutical composition comprising one or a combination of two or more compounds or tautomers, stereoisomers, solvates, metabolites, isotopically-labeled compounds, pharmaceutically acceptable salts or co-crystals thereof of claim 1.

27. Use of A method for preventing and treating a disease mediated respectively or synergistically by LSD1 and/or HDAC, comprising administering to a subject in need thereof the compound or tautomer, stereoisomer, solvate, metabolite, isotopically-labeled compound, pharmaceutically acceptable salt or co-crystal thereof of claim 1; preferably for preventing and treating a disease mediated by LSD1 and/or HDAC;
preferably, the HDAC enzyme comprises isoforms of HDAC1, HDAC2, HDAC3, HDAC4, HDAC5, HDAC6, HDAC7, HDAC8; preferably the HDAC enzyme is selected from HDAC1 or HDAC8 isoform, further preferably the HDAC enzyme is HDAC1 isoform;
preferably, the disease is cancer or autoimmune disease;
preferably, the cancer is selected from: non-small cell lung cancer, small cell lung cancer, pancreatic cancer, ovarian cancer, bladder cancer, prostate cancer, chronic myeloid leukemia, colorectal cancer, brain cancer, liver cancer, kidney cancer, gastric cancer, breast cancer, triple negative breast cancer, skin cancer, melanoma, head and neck cancer, bone cancer, cervical cancer, pelvic cancer, vaginal cancer, oral cancer, lymphoma, blood cancer, esophageal cancer, urethral cancer, nasal cavity cancer.

28. (canceled)

29. (canceled)

30. A pharmaceutical composition comprising one or a combination of two or more compounds or tautomers, stereoisomers, solvates, metabolites, isotopically-labeled compounds, pharmaceutically acceptable salts or co-crystals thereof of claim 17.

31. A pharmaceutical composition comprising one or a combination of two or more compounds or tautomers, stereoisomers, solvates, metabolites, isotopically-labeled compounds, pharmaceutically acceptable salts or co-crystals thereof of claim 25.

32. A method for preventing and treating a disease mediated respectively or synergistically by LSD1 and/or HDAC, comprising administering to a subject in need thereof the compound or tautomer, stereoisomer, solvate, metabolite, isotopically-labeled compound, pharmaceutically acceptable salt or co-crystal thereof of claim 17; preferably for preventing and treating a disease mediated by LSD1 and/or HDAC;
preferably, the HDAC enzyme comprises isoforms of HDAC1, HDAC2, HDAC3, HDAC4, HDAC5, HDAC6, HDAC7, HDAC8; preferably, the HDAC enzyme is selected from HDAC1 or HDAC8 isoform, further preferably the HDAC enzyme is HDAC1 isoform;
preferably, the disease is cancer or autoimmune disease;
preferably, the cancer is selected from: non-small cell lung cancer, small cell lung cancer, pancreatic cancer, ovarian cancer, bladder cancer, prostate cancer, chronic myeloid leukemia, colorectal cancer, brain cancer, liver cancer, kidney cancer, gastric cancer, breast cancer, triple negative breast cancer, skin cancer, melanoma, head and neck cancer, bone cancer, cervical cancer, pelvic cancer, vaginal cancer, oral cancer, lymphoma, blood cancer, esophageal cancer, urethral cancer, nasal cavity cancer.

33. A method for preventing and treating a disease mediated respectively or synergistically by LSD1 and/or HDAC, comprising administering to a subject in need thereof the compound or tautomer, stereoisomer, solvate, metabolite, isotopically-labeled compound, pharmaceutically acceptable salt or co-crystal thereof of claim 25; preferably for preventing and treating a disease mediated by LSD1 and/or HDAC;
preferably, the HDAC enzyme comprises isoforms of HDAC1, HDAC2, HDAC3, HDAC4, HDAC5, HDAC6, HDAC7, HDAC8; preferably, the HDAC enzyme is selected from HDAC1 or HDAC8 isoform, further preferably the HDAC enzyme is HDAC1 isoform;
preferably, the disease is cancer or autoimmune disease;
preferably, the cancer is selected from: non-small cell lung cancer, small cell lung cancer, pancreatic cancer, ovarian cancer, bladder cancer, prostate cancer, chronic myeloid leukemia, colorectal cancer, brain cancer, liver cancer, kidney cancer, gastric cancer, breast cancer, triple negative breast cancer, skin cancer, melanoma, head and neck cancer, bone cancer, cervical cancer, pelvic cancer, vaginal cancer, oral cancer, lymphoma, blood cancer, esophageal cancer, urethral cancer, nasal cavity cancer.
